(19) United States
(12) Patent Application Publication
(10) Pub. No.: US 2003/0073207 A1
(43) Pub. Date: Apr. 17, 2003

(54) Enzymatic nucleic acid treatment of diseases or conditions related to levels of epidermal growth factor receptors

(76) Inventors: Saghir Akhtar, Safat (KW); James McSwiggen, Boulder, CO (US)

Correspondence Address: MCDONNELL BOEHNEN HULBERT & BERGHOFF, 300 SOUTH WACKER DRIVE, SUITE 3200, CHICAGO, IL, 60606, US

(21) Appl. No.: 09/848,754

(22) Filed: May 3, 2001

Related U.S. Application Data
(63) Continuation-in-part of application No. 09/401,063, filed on Sep. 22, 1999.
Application No. 09/401,063 is a continuation of application No. 08/985,162, filed on Dec. 4, 1997, now Pat. No. 6,057,156.
(60) Provisional application No. 60/036,476, filed on Jan. 31, 1997.

(51) Int. Cl. A61K 48/00; C07H 21/02; C12N 9/00

(52) U.S. Cl. 435/183; 536/23.1; 514/44

(57) ABSTRACT

The present invention relates to nucleic acid molecules, including antisense and enzymatic nucleic acid molecules, such as hammerhead ribozymes, DNAzymes, allozymes and antisense, which modulate the expression of epidermal growth factor receptor genes.

This patent application is a continuation-in-part of Akhtar et al., U.S. Ser. No. 09/401,063, filed Sep. 22, 1999, entitled “ENZYMATIC NUCLEIC ACID TREATMENT OF DISEASES OR CONDITIONS RELATED TO LEVELS OF EPIDERMAL GROWTH FACTOR” which is a continuation application of Akhtar et al., U.S. Ser. No. 08/985,162, filed Dec. 4, 1997 now U.S. Pat. No. 6,057,156 entitled “ENZYMATIC NUCLEIC ACID TREATMENT OF DISEASES OR CONDITIONS RELATED TO LEVELS OF EPIDERMAL GROWTH FACTOR”, which claims priority from Akhtar et al., USS No. 60/036,749, filed Jan. 31, 1997, entitled “ENZYMATIC NUCLEIC ACID TREATMENT OF DISEASES OR CONDITIONS RELATED TO LEVELS OF EPIDERMAL GROWTH FACTOR” These application are hereby incorporated by reference herein in their entirety including the drawings.

### BACKGROUND OF THE INVENTION

The present invention relates to therapeutic compositions and methods for the treatment or diagnosis of diseases or conditions related to EGFR expression levels, such as cancer. The discussion is not meant to be complete and is provided only for understanding of the invention that follows. This summary is not an admission that any of the work described below is prior art to the claimed invention.

The epidermal growth factor receptor (EGFR) is a 170 kDa transmembrane glycoprotein consisting of an extracellular ‘ligand’ binding domain, a transmembrane region and an intracellular domain with tyrosine kinase activity (Kung et al., 1994). The binding of growth factors to the EGFR results in down regulation of the ligand-receptor complex, autophosphorylation of the receptor and other protein substrates, leading ultimately to DNA synthesis and cell division. The external ligand binding domain is stimulated by EGF and also by TGFa, amphiregulin and some viral growth factors (Nodjtahedi & Dean, 1994).

One of the striking characteristics of the EGFR gene (c-erbBl), located on chromosome 7, is it's homology to the avian erythroblastosis virus oncogene (v-erbB), which induces malignancies in chickens. The v-erbB gene codes for a truncated product that lacks the extracellular ligand binding domain. The tyrosine kinase domain of the EGFR has been found to have 97% homology to the v-erbB transforming protein (Downward et al., 1984).

Recent studies have shown that the EGFR is overexpressed in a number of malignant human tissues when compared to their normal tissue counterparts (for review see Khazaie et al., 1993). An important finding has been the discovery that the gene for the receptor is both amplified and overexpressed in a number of cancer cells. Overexpression of the EGFR is often accompanied by the co-expression of the growth factors EGF and TGFa, suggesting that an autocrine pathway for control of growth may play a major part in the progression of tumors (Sporn & Roberts, 1985). It is now widely believed that this is a mechanism by which tumor cells can escape normal physiological control.

Growth factors and their receptors appear to have an important role in the development of human brain tumors. A high incidence of overexpression, amplification, deletion and structural rearrangement of the gene coding for the EGFR has been found in biopsies of brain tumors (Ostrowski et al., 1994). In fact the amplification of the EGFR gene in glioblastoma multiforme tumors is one of the most consistent genetic alterations known, with the EGFR being overexpressed in approximately 40% of malignant gliomas (Black, 1991). It has also been demonstrated that in 50% of glioblastomas, amplification of the EGFR gene is accompanied by the co-expression of mRNA for at least one or both of the growth factors EGF and TNFa (Ekstrand et al., 1991).

The amplified genes are frequently rearranged and associated with polymorphism leading to abnormal protein products (Wong et al., 1994). The rearrangements that have been characterized usually show deletions of part of the extracellular domain, resulting in the production of an EGFR protein that is smaller in size. Three classes of deletion mutant EGF receptor genes have been identified in glioblastoma tumors. Type I mutants lack the majority of the external domain, including the ligand binding site, type II mutants have a deletion in the domain adjacent to the membrane but can still bind ligands and type III, which is the most common and found in 17% of glioblastomas, have a deletion of 267 amino acids spanning domains I and II of the EGFR.

In addition to glioblastomas, abnormal EGFR expression has also been reported in a number of squamous epidermoid cancers and breast cancers (reviewed in Kung et al, 1994; Modjtahedi & Dean, 1994). Interestingly, evidence also suggests that many patients with tumors that overexpress the EGFR have a poorer prognosis than those who do not (Khazaie et al., 1993). Consequently, therapeutic strategies which can potentially inhibit or reduce the aberrant expression of the EGFR receptor are of great interest as potential anti-cancer agents.

Akhtar et al., U.S. Pat. No. 6,057,156, describe enzymatic nucleic acid molecules targeting epidermal growth factor receptors. Akhtar et al., International PCT publication No. WO 98/33893, describe enzymatic nucleic acid molecules targeting epidermal growth factor receptors.

Halatsch et al., 2000, J. Neurosurg., 92, 297-305, describe specific hairpin ribozymes targeting specific epidermal growth factor receptors.

Yamazaki et al., 1998, J. Natl. Cancer Inst., 90, 581-587, describe specific hammerhead ribozymes targeting specific epidermal growth factor receptors.

Fell et al., 1997, Antisense Nucleic Acid Drug Dev., 7, 319-326, describe 2′-amino and 2′-O-methyl modified chimeric hammerhead ribozymes targeting epidermal growth factor receptor mRNA. Yamazaki et al., 1995, PAACREAM, 36, 449, abstract No. 2556, describes a plasmid vector expressed hammerhead ribozyme targeted against a specific target site withing a specific mutant EGFR RNA.

Ludwig and Sproat, International PCT Publication No. WO 97/18312, describe a ribozyme with specific chemical modifications targeting EGFR.

Pyle and Michels, International PCT Publication No. WO 96/22689, describe specific group II intron based ribozymes targeting EGFR.

### SUMMARY OF THE INVENTION

The invention features novel nucleic acid-based molecules, for example, enzymatic nucleic acid molecules, antisense nucleic acids, 2-5A antisense chimeras, triplex DNA, decoy RNA, aptamers, and antisense nucleic acids containing RNA cleaving chemical groups, and methods to modulate gene expression, for example, genes encoding epidermal growth factor receptors. In particular, the instant invention features nucleic-acid based molecules and methods to modulate the expression of epidermal growth factor receptors (EGFR).

The invention features one or more nucleic acid-based molecules and methods that independently or in combination modulate the expression of gene(s) encoding epidermal growth factor receptors. In particular embodiments, the invention features nucleic acid-based molecules and methods that modulate the expression of EGFR gene, for example (Genbank Accession No. NM_{—}005228).

The invention also features nucleic acid-based molecules and methods that modulate the expression of genes encoding epidermal growth factor receptor (EGFR), for example, Genbank Accession No. NM005228, and ERBB2/HER2/NEU, for example, Genbank Accession Nos. NM_{—}004448 and X03363.

The description below of the various aspects and embodiments is provided with reference to the exemplary epidermal growth receptor (EGFR) gene, also recognized as ERBB or HER1. However, the various aspects and embodiments are also directed to other genes which express EGFR proteins and other receptors involved in oncogenesis. Those additional genes can be analyzed for target sites using the methods described for EGFR. Thus, the inhibition and the effects of such inhibition of the other genes can be performed as described herein.

In one embodiment, the invention features the use of an enzymatic nucleic acid molecule, preferably in the hammerhead, NCH, G-cleaver, amberzyme, zinzyme and/or DNAzyme motif, to down-regulate the expression of EGFR genes.

By “inhibit” or “down-regulate” it is meant that the expression of the gene, or level of RNAs or equivalent RNAs encoding one or more protein subunits, or activity of one or more protein subunits, such as EGFR subunit(s), is reduced below that observed in the absence of the nucleic acid molecules of the invention. In one embodiment, inhibition or down-regulation with enzymatic nucleic acid molecule preferably is below that level observed in the presence of an enzymatically inactive or attenuated molecule that is able to bind to the same site on the target RNA, but is unable to cleave that RNA. In another embodiment, inhibition or down-regulation with antisense oligonucleotides is preferably below that level observed in the presence of, for example, an oligonucleotide with scrambled sequence or with mismatches. In another embodiment, inhibition or down-regulation of EGFR with the nucleic acid molecule of the instant invention is greater in the presence of the nucleic acid molecule than in its absence.

By “up-regulate” is meant that the expression of the gene, or level of RNAs or equivalent RNAs encoding one or more protein subunits, or activity of one or more protein subunits, such as EGFR subunit(s), is greater than that observed in the absence of the nucleic acid molecules of the invention. For example, the expression of a gene, such as EGFR gene, can be increased in order to treat, prevent, ameliorate, or modulate a pathological condition caused or exacerbated by an absence or low level of gene expression.

By “modulate” is meant that the expression of the gene, or level of RNAs or equivalent RNAs encoding one or more protein subunits, or activity of one or more protein subunit(s) is up-regulated or down-regulated, such that the expression, level, or activity is greater than or less than that observed in the absence of the nucleic acid molecules of the invention.

By “enzymatic nucleic acid molecule” it is meant a nucleic acid molecule which has complementarity in a substrate binding region to a specified gene target, and also has an enzymatic activity which is active to specifically cleave target RNA. That is, the enzymatic nucleic acid molecule is able to intermolecularly cleave RNA and thereby inactivate a target RNA molecule. These complementary regions allow sufficient hybridization of the enzymatic nucleic acid molecule to the target RNA and thus permit cleavage. One hundred percent complementarity is preferred, but complementarity as low as 50-75% can also be useful in this invention (see for example Werner and Uhlenbeck, 1995, Nucleic Acids Research, 23, 2092-2096; Hammann et al., 1999, Antisense and Nucleic Acid Drug Dev., 9, 25-31). The nucleic acids can be modified at the base, sugar, and/or phosphate groups. The term enzymatic nucleic acid is used interchangeably with phrases such as ribozymes, catalytic RNA, enzymatic RNA, catalytic DNA, aptazyme or aptamer-binding ribozyme, regulatable ribozyme, catalytic oligonucleotides, nucleozyme, DNAzyme, RNA enzyme, endoribonuclease, endonuclease, minizyme, leadzyme, oligozyme or DNA enzyme. All of these terminologies describe nucleic acid molecules with enzymatic activity. The specific enzymatic nucleic acid molecules described in the instant application are not limiting in the invention and those skilled in the art will recognize that all that is important in an enzymatic nucleic acid molecule of this invention is that it has a specific substrate binding site which is complementary to one or more of the target nucleic acid regions, and that it have nucleotide sequences within or surrounding that substrate binding site which impart a nucleic acid cleaving and/or ligation activity to the molecule (Cech et al., U.S. Pat. No. 4,987,071; Cech et al., 1988, 260 JAMA 3030).

By “nucleic acid molecule” as used herein is meant a molecule having nucleotides. The nucleic acid can be single, double, or multiple stranded and can comprise modified or unmodified nucleotides or non-nucleotides or various mixtures and combinations thereof.

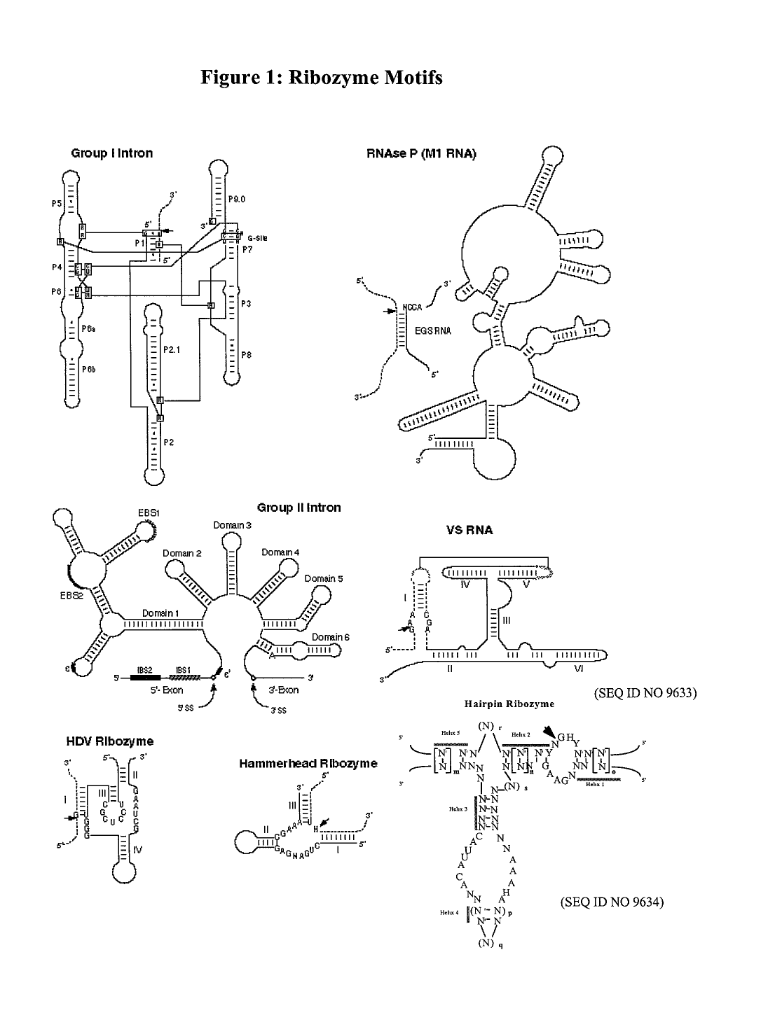

By “enzymatic portion” or “catalytic domain” is meant that portion/region of the enzymatic nucleic acid molecule essential for cleavage of a nucleic acid substrate (for example see FIG. 1).

By “substrate binding arm” or “substrate binding domain” is meant that portion/region of a enzymatic nucleic acid which is able to interact, for example via complementarity (i.e., able to base-pair with), with a portion of its substrate. Preferably, such complementarity is 100%, but can be less if desired. For example, as few as 10 bases out of 14 can be base-paired (see for example Werner and Uhlenbeck, 1995, Nucleic Acids Research, 23, 2092-2096; Hammann et al., 1999, Antisense and Nucleic Acid Drug Dev., 9, 25-31). Examples of such arms are shown generally in FIGS. 1-4. That is, these arms contain sequences within a enzymatic nucleic acid which are intended to bring enzymatic nucleic acid and target RNA together through complementary base-pairing interactions. The enzymatic nucleic acid of the invention can have binding arms that are contiguous or non-contiguous and may be of varying lengths. The length 22 of the binding arm(s) are preferably greater than or equal to four nucleotides and of sufficient length to stably interact with the target RNA; preferably 12-100 nucleotides; more preferably 14-24 nucleotides long (see for example Werner and Uhlenbeck, supra; Hamman et al., supra; Hampel et al., EP0360257; Berzal-Herrance et al., 1993, EMBO J., 12, 2567-73). If two binding arms are chosen, the design is such that the length of the binding arms are symmetrical (i.e., each of the binding arms is of the same length; e.g., five and five nucleotides, or six and six nucleotides, or seven and seven nucleotides long) or asymmetrical (i.e., the binding arms are of different length; e.g., six and three nucleotides; three and six nucleotides long; four and five nucleotides long; four and six nucleotides long; four and seven nucleotides long; and the like).

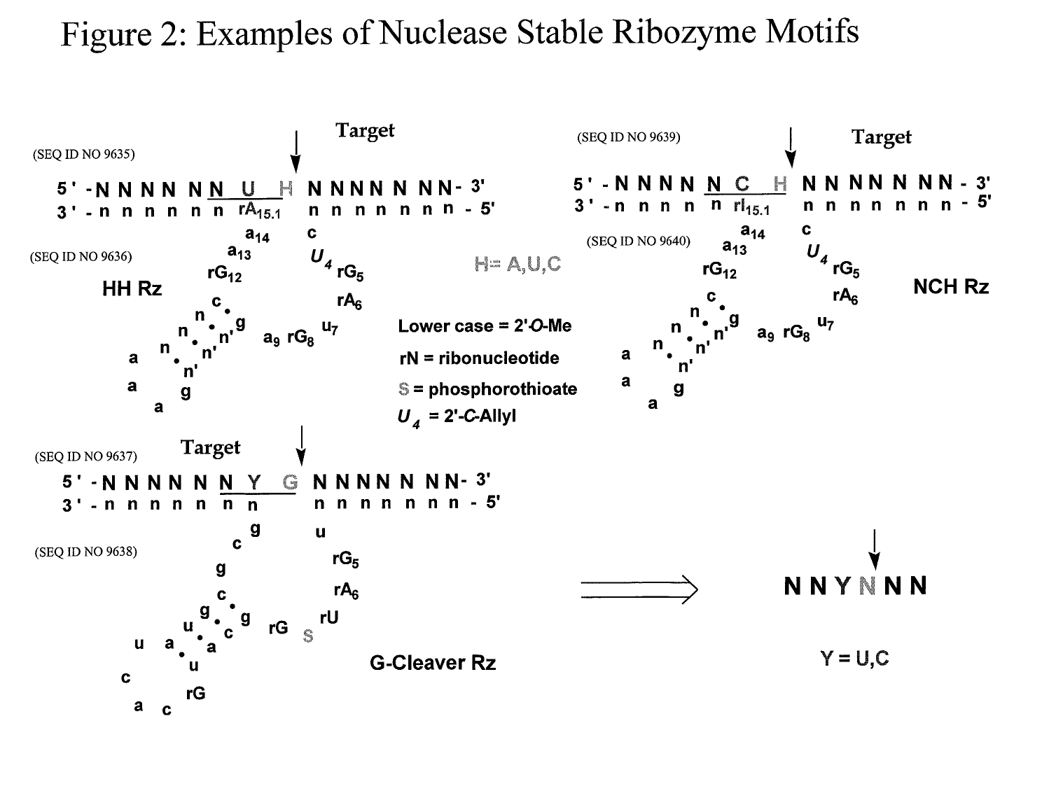

By “Inozyme” or “NCH” motif or configuration is meant, an enzymatic nucleic acid molecule comprising a motif as is generally described as NCH Rz in FIG. 2. Inozymes possess endonuclease activity to cleave RNA substrates having a cleavage triplet NCH/, where N is a nucleotide, C is cytidine and H is adenosine, uridine or cytidine, and/represents the cleavage site. H is used interchangeably with X. Inozymes can also possess endonuclease activity to cleave RNA substrates having a cleavage triplet NCN/, where N is a nucleotide, C is cytidine, and/represents the cleavage site. “I” in FIG. 2 represents an Inosine nucleotide, preferably a ribo-Inosine or xylo-Inosine nucleoside.

By “G-cleaver” motif or configuration is meant, an enzymatic nucleic acid molecule comprising a motif as is generally described as G-cleaver Rz in FIG. 2. G-cleavers possess endonuclease activity to cleave RNA substrates having a cleavage triplet NYN/, where N is a nucleotide, Y is uridine or cytidine and/represents the cleavage site. G-cleavers can be chemically modified as is generally shown in FIG. 2.

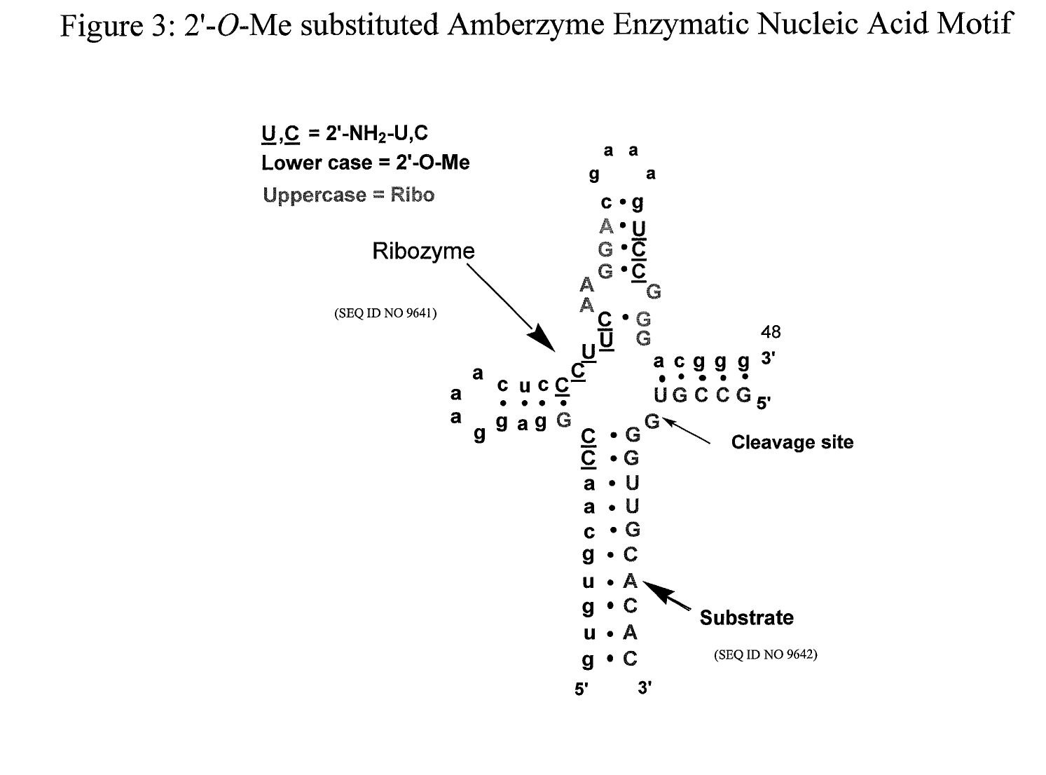

By “amberzyme” motif or configuration is meant, an enzymatic nucleic acid molecule comprising a motif as is generally described in FIG. 3. Amberzymes possess endonuclease activity to cleave RNA substrates having a cleavage triplet NGIN, where N is a nucleotide, G is guanosine, and/represents the cleavage site. Amberzymes can be chemically modified to increase nuclease stability through substitutions as are generally shown in FIG. 3. In addition, differing nucleoside and/or non-nucleoside linkers can be used to substitute the 5′-gaaa-3′ loops 1:; shown in the figure. Amberzymes represent a non-limiting example of an enzymatic nucleic acid molecule that does not require a ribonucleotide (2′-OH) group within its own nucleic acid sequence for activity.

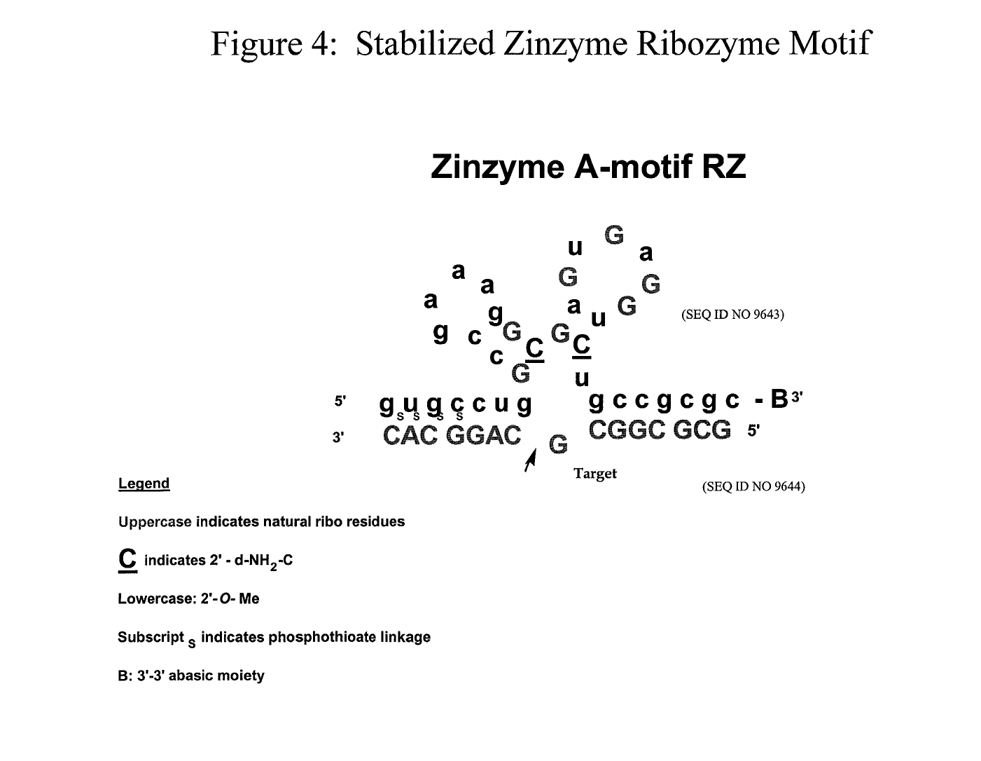

By “zinzyme” motif or configuration is meant, an enzymatic nucleic acid molecule comprising a motif as is generally described in FIG. 4. Zinzymes possess endonuclease activity to cleave RNA substrates having a cleavage triplet including but not limited to YG/Y, where Y is uridine or cytidine, and G is guanosine and/represents the cleavage site. Zinzymes can be chemically modified to increase nuclease stability through substitutions as are generally shown in FIG. 4, including substituting 2′-O-methyl guanosine nucleotides for guanosine nucleotides. In addition, differing nucleotide and/or non-nucleotide linkers can be used to substitute the 5′-gaaa-2′ loop shown in the figure. Zinzymes represent a non-limiting example of an enzymatic nucleic acid molecule that does not require a ribonucleotide (2′-OH) group within its own nucleic acid sequence for activity.

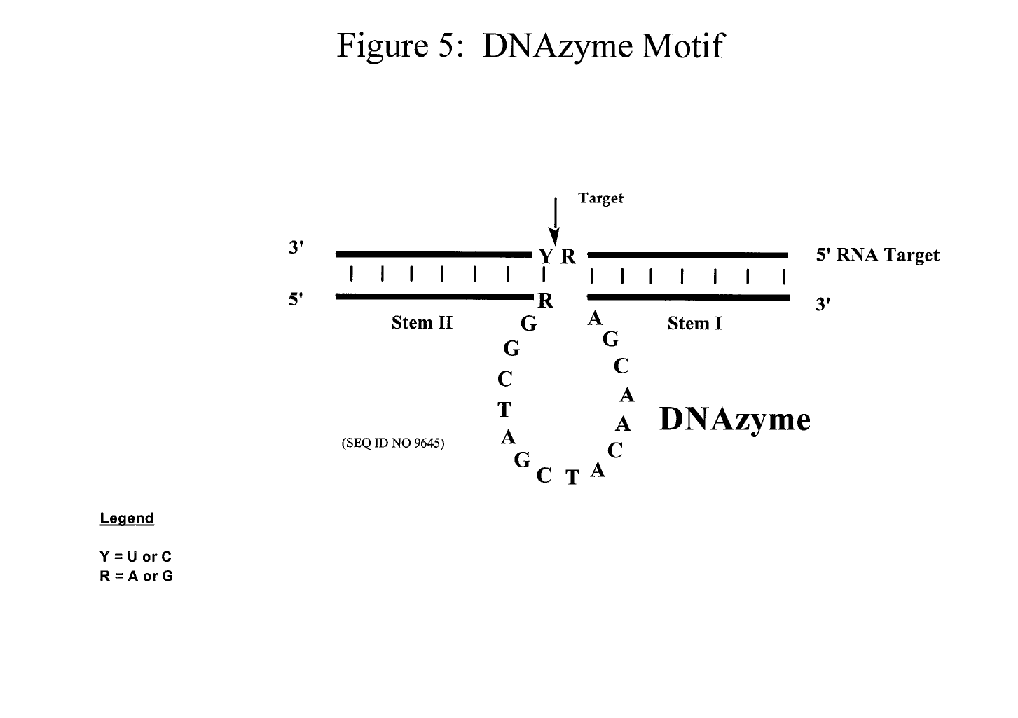

By ‘DNAzyme’ is meant, an enzymatic nucleic acid molecule that does not require the presence of a 2′-OH group for its activity. In particular embodiments the enzymatic nucleic acid molecule can have an attached linker(s) or other attached or associated groups, moieties, or chains containing one or more nucleotides with 2′-OH groups. DNAzymes can be synthesized chemically or expressed endogenously in vivo, by means of a single stranded DNA vector or equivalent thereof. An example of a DNAzyme is shown in FIG. 5 and is generally reviewed in Usman et al., International PCT Publication No. WO 95/11304; Chartrand et al, 1995, NAR 23, 4092; Breaker et al., 1995, Chem. Bio. 2, 655; Santoro et al., 1997, PNAS 94, 4262; Breaker, 1999, Nature Biotechnology, 17, 422-423; and Santoro et. al., 2000, J. Am. Chem. Soc., 122, 2433-39. Additional DNAzyme motifs can be selected for using techniques similar to those described in these references, and hence, are within the scope of the present invention.

By “sufficient length” is meant an oligonucleotide of greater than or equal to 3 nucleotides that is of a length great enough to provide the intended function under the expected condition. For example, for binding arms of enzymatic nucleic acid “sufficient length” means that the binding arm sequence is long enough to provide stable binding to a target site under the expected binding conditions. Preferably, the binding arms are not so long as to prevent useful turnover of the nucleic acid molecule.

By “stably interact” is meant interaction of the oligonucleotides with target nucleic acid (e.g., by forming hydrogen bonds with complementary nucleotides in the target under physiological conditions) that is sufficient to the intended purpose (e.g., cleavage of target RNA by an enzyme).

By “equivalent” RNA to EGFR is meant to include those naturally occurring RNA molecules having homology (partial or complete) to EGFR proteins or encoding for proteins with similar function as EGFR proteins in various organisms, including human, rodent, primate, Arabbit, pig, protozoans, fungi, plants, and other microorganisms and parasites. The equivalent RNA sequence also includes in addition to the coding region, regions such as 5′-untranslated region, 3′-untranslated region, introns, intron-exon junction and the like.

By “homology” is meant the nucleotide sequence of two or more nucleic acid molecules is partially or completely identical.

By “antisense nucleic acid”, it is meant a non-enzymatic nucleic acid molecule that binds to target RNA by means of RNA-RNA or RNA-DNA or RNA-PNA (protein nucleic acid; Egholm et al., 1993 Nature 365, 566) interactions and alters the activity of the target RNA (for a review, see Stein and Cheng, 1993 Science 261, 1004 and Woolf et al., U.S. Pat. No. 5,849,902). Typically, antisense molecules are complementary to a target sequence along a single contiguous sequence of the antisense molecule. However, in certain embodiments, an antisense molecule can bind to substrate such that the substrate molecule forms a loop, and/or an antisense molecule can bind such that the antisense molecule forms a loop. Thus, the antisense molecule can be complementary to two (or even more) non-contiguous substrate sequences or two (or even more) non-contiguous sequence portions of an antisense molecule can be complementary to a target sequence or both. For a review of current antisense strategies, see Schmajuk et al., 1999, J. Biol. Chem., 274, 21783-21789, Delihas et al., 1997, Nature, 15, 751-753, Stein et al., 1997, Antisense N. A. Drug Dev., 7, 151, Crooke, 2000, Methods Enzymol., 313, 3-45; Crooke, 1998, Biotech. Genet. Eng. Rev., 15, 121-157, Crooke, 1997, Ad. Pharmacol., 40, 1-49. In addition, antisense DNA can be used to target RNA by means of DNA-RNA interactions, thereby activating RNase H, which digests the target RNA in the duplex. The antisense oligonucleotides can comprise one or more RNAse H activating region, which is capable of activating RNAse H cleavage of a target RNA. Antisense DNA can be synthesized chemically or expressed via the use of a single stranded DNA expression vector or equivalent thereof.

By “RNase H activating region” is meant a region (generally greater than or equal to 4-25 nucleotides in length, preferably from 5-11 nucleotides in length) of a nucleic acid molecule capable of binding to a target RNA to form a non-covalent complex that is recognized by cellular RNase H enzyme (see for example Arrow et al., U.S. Pat. No. 5,849,902; Arrow et al., U.S. Pat. No. 5,989,912). The RNase H enzyme binds to the nucleic acid molecule-target RNA complex and cleaves the target RNA sequence. The RNase H activating region comprises, for example, phosphodiester, phosphorothioate (preferably at least four of the nucleotides are phosphorothiote substitutions; more specifically, 4-11 of the nucleotides are phosphorothiote substitutions); phosphorodithioate, 5′-thiophosphate, or methylphosphonate backbone chemistry or a combination thereof. In addition to one or more backbone chemistries described above, the RNase H activating region can also comprise a variety of sugar chemistries. For example, the RNase H activating region can comprise deoxyribose, arabino, fluoroarabino or a combination thereof, nucleotide sugar chemistry. Those skilled in the art will recognize that the foregoing are non-limiting examples and that any combination of phosphate, sugar and base chemistry of a nucleic acid that supports the activity of RNase H enzyme is within the scope of the definition of the RNase H activating region and the instant invention.

By “2-5A antisense chimera” is meant an antisense oligonucleotide containing a 5′-phosphorylated 2′-5′-linked adenylate residue. These chimeras bind to target RNA in a sequence-specific manner and activate a cellular 2-SA-dependent ribonuclease which, in turn, cleaves the target RNA (Torrence et al., 1993 Proc. Natl. Acad. Sci. USA 90, 1300; Silverman et al., 2000, Methods Enzymol., 313, 522-533; Player and Torrence, 1998, Pharmacol. Ther., 78, 55-113).

By “triplex forming oligonucleotides” is meant an oligonucleotide that can bind to a double-stranded DNA in a sequence-specific manner to form a triple-strand helix. Formation of such triple helix structure has been shown to inhibit transcription of the targeted gene (Duval-Valentin et al., 1992 Proc. Natl. Acad. Sci. USA 89, 504; Fox, 2000, Curr. Med. Chem., 7, 17-37; Praseuth et. al, 2000, Biochim. Biophys. Acta, 1489, 181-206).

By “gene” it is meant a nucleic acid that encodes an RNA, for example, nucleic acid sequences including but not limited to structural genes encoding a polypeptide.

“Complementarity” refers to the ability of a nucleic acid to form hydrogen bond(s) with another RNA sequence by either traditional Watson-Crick or other non-traditional types. In reference to the nucleic molecules of the present invention, the binding free energy for a nucleic acid molecule with its target or complementary sequence is sufficient to allow the relevant function of the nucleic acid to proceed, e.g., enzymatic nucleic acid cleavage, antisense or triple helix inhibition. Determination of binding free energies for nucleic acid molecules is well known in the art (see, e.g., Turner et al., 1987, CSH Symp. Quant. Biol. LII pp.123-133; Frier et al., 1986, Proc. Nat. Acad. Sci. USA 83:9373-9377; Turner et al., 1987, J. Am. Chem. Soc. 109:3783-3785). A percent complementarity indicates the percentage of contiguous residues in a nucleic acid molecule which can form hydrogen bonds (e.g., Watson-Crick base pairing) with a second nucleic acid sequence (e.g., 5, 6, 7, 8, 9, 10 out of 10 being 50%, 60%, 70%, 80%, 90%, and 100% complementary). “Perfectly complementary” means that all the contiguous residues of a nucleic acid sequence will hydrogen bond with the same number of contiguous residues in a second nucleic acid sequence.

By “RNA” is meant a molecule comprising at least one ribonucleotide residue. By “ribonucleotide” or “2′-OH” is meant a nucleotide with a hydroxyl group at the 2′ position of a β-D-ribo-furanose moiety.

By “decoy RNA” is meant an RNA molecule or aptamer that is designed to preferentially bind to a predetermined ligand. Such binding can result in the inhibition or activation of a target molecule. The decoy RNA or aptamer can compete with a naturally occuring binding target for the binding of a specific ligand. For example, it has been shown that over-expression of HIV trans-activation response (TAR) RNA can act as a “decoy” and efficiently binds HIV tat protein, thereby preventing it from binding to TAR sequences encoded in the HIV RNA (Sullenger et al., 1990, Cell, 63, 601-608). This is but a specific example and those in the art will recognize that other embodiments can be readily generated using techniques generally known in the art, see for example Gold et al., 1995, Annu. Rev. Biochem., 64, 763; Brody and Gold, 2000, J. Biotechnol., 74, 5; Sun, 2000, Curr. Opin. Mol. Ther., 2, 100; Kusser, 2000, J. Biotechnol., 74, 27; Hermann and Patel, 2000, Science, 287, 820; and Jayasena, 1999, Clinical Chemistry, 45, 1628. Similarly, a decoy RNA can be designed to bind to a EGFR receptor and block the binding of EGFR or a decoy RNA can be designed to bind to EGFR and prevent interaction with the EGFR receptor.

Several varieties of naturally-occurring enzymatic RNAs are known presently. Each can catalyze the hydrolysis of RNA phosphodiester bonds in trans (and thus can cleave other RNA molecules) under physiological conditions. Table I summarizes some of the characteristics of these ribozymes. In general, enzymatic nucleic acids act by first binding to a target RNA. Such binding occurs through the target binding portion of a enzymatic nucleic acid which is held in close proximity to an enzymatic portion of the molecule that acts to cleave the target RNA. Thus, the enzymatic nucleic acid first recognizes and then binds a target RNA through complementary base-pairing, and once bound to the correct site, acts enzymatically to cut the target RNA. Strategic cleavage of such a target RNA will destroy its ability to direct synthesis of an encoded protein. After an enzymatic nucleic acid has bound and cleaved its RNA target, it is released from that RNA to search for another target and can repeatedly bind and cleave new targets. Thus, a single ribozyme molecule is able to cleave many molecules of target RNA. In addition, the ribozyme is a highly specific inhibitor of gene expression, with the specificity of inhibition depending not only on the base-pairing mechanism of binding to the target RNA, but also on the mechanism of target RNA cleavage. Single mismatches, or base-substitutions, near the site of cleavage can completely eliminate catalytic activity of a ribozyme.

The enzymatic nucleic acid molecule that cleave the specified sites in EGFR-specific RNAs represent a novel therapeutic approach to treat a variety of cancers, including but not limited to breast, lung, prostate, colorectal, brain, esophageal, bladder, pancreatic, cervical, head and neck, and ovarian cancer, melanoma, lymphoma, glioma, multidrug resistant cancers, and/or other cancers which respond to the modulation of EGFR expression.

In one embodiment of the inventions described herein, the enzymatic nucleic acid molecule is formed in a hammerhead or hairpin motif, but can also be formed in the motif of a hepatitis delta virus, group I intron, group II intron or RNase P RNA (in association with an RNA guide sequence), Neurospora VS RNA, DNAzymes, NCH cleaving motifs, or G-cleavers. Examples of such hammerhead motifs are described by Dreyfus, supra, Rossi et al., 1992, AIDS Research and Human Retroviruses 8, 183; of hairpin motifs by Hampel et al., EP0360257, Hampel and Tritz, 1989 Biochemistry 28, 4929, Feldstein et al., 1989, Gene 82, 53, Haseloff and Gerlach, 1989, Gene, 82, 43, and Hampel et al., 1990 Nucleic Acids Res. 18, 299; Chowrira & McSwiggen, U.S. Pat. No. 5,631,359; of the hepatitis delta virus motif is described by Perrotta and Been, 1992 Biochemistry 31, 16; of the RNase P motif by Guerrier-Takada et al., 1983 Cell 35, 849; Forster and Altman, 1990, Science 249, 783; Li and Altman, 1996, Nucleic Acids Res. 24, 835; Neurospora VS RNA ribozyme motif is described by Collins (Saville and Collins, 1990 Cell 61, 685-696; Saville and Collins, 1991 Proc. Natl. Acad. Sci. USA 88, 8826-8830; Collins and Olive, 1993 Biochemistry 32, 2795-2799; Guo and Collins, 1995, EMBO. J. 14, 363); Group II introns are described by Griffin et al., 1995, Chem. Biol. 2, 761; Michels and Pyle, 1995, Biochemistry 34, 2965; Pyle et al., International PCT Publication No. WO 96/22689; of the Group I intron by Cech et aL, U.S. Pat. No. 4,987,071 and of DNAzymes by Usman et al., International PCT Publication No. WO 95/11304; Chartrand et al., 1995, NAR 23, 4092; Breaker et al., 1995, Chem. Bio. 2, 655; Santoro et al., 1997, PNAS 94, 4262, and Beigelman et al., International PCT publication No. WO 99/55857. NCH cleaving motifs are described in Ludwig & Sproat, International PCT Publication No. WO 98/58058; and G-cleavers are described in Kore et al., 1998, Nucleic Acids Research 26, 4116-4120 and Eckstein et al., International PCT Publication No. WO 99/16871. Additional motifs such as the Aptazyme (Breaker et al., WO 98/43993), Amberzyme (Class I motif; FIG. 3; Beigelman et al., U.S. Serial No. 09/301,511) and Zinzyme (FIG. 4) (Beigelman et al., U.S. Ser. No. 09/301,511), all included by reference herein including drawings, can also be used in the present invention. These specific motifs or configurations are not limiting in the invention and those skilled in the art will recognize that all that is important in an enzymatic nucleic acid molecule of this invention is that it has a specific substrate binding site which is complementary to one or more of the target gene RNA regions, and that it have nucleotide sequences within or surrounding that substrate binding site which impart an RNA cleaving activity to the molecule (Cech et al., U.S. Pat. No. 4,987,071).

In one embodiment of the present invention, a nucleic acid molecule of the instant invention can be between 12 and 100 nucleotides in length. Exemplary enzymatic nucleic acid molecules of the invention are shown in Table III-VIII. For example, enzymatic nucleic acid molecules of the invention are preferably between 15 and 50 nucleotides in length, more preferably between 25 and 40 nucleotides in length, e.g., 34, 36, or 38 nucleotides in length (for example see Jarvis et al., 1996, J. Biol. Chem., 271, 29107-29112). Exemplary DNAzymes of the invention are preferably between 15 and 40 nucleotides in length, more preferably between 25 and 35 nucleotides in length, e.g., 29, 30, 31, or 32 nucleotides in length (see for example Santoro et al., 1998, Biochemistry, 37, 13330-13342; Chartrand et al., 1995, Nucleic Acids Research, 23, 4092-4096). Exemplary antisense molecules of the invention are preferably between 15 and 75 nucleotides in length, more preferably between 20 and 35 nucleotides in length, e.g., 25, 26, 27, or 28 nucleotides in length (see for example Woolf et al., 1992, PNAS., 89, 7305-7309; Milner et al., 1997, Nature Biotechnology, 15, 537-541). Exemplary triplex forming oligonucleotide molecules of the invention are preferably between 10 and 40 nucleotides in length, more preferably between 12 and 25 nucleotides in length, e.g., 18, 19, 20, or 21 nucleotides in length (see for example Maher et al., 1990, Biochemistry, 29, 8820-8826; Strobel and Dervan, 1990, Science, 249, 73-75). Those skilled in the art will recognize that all that is required is for the nucleic acid molecule are of length and conformation sufficient and suitable for the nucleic acid molecule to catalyze a reaction contemplated herein. The length of the nucleic acid molecules of the instant invention are not limiting within the general limits stated.

Preferably, a nucleic acid molecule that modulates, for example, down-regulates EGFR replication or expression comprises between 12 and 100 bases complementary to a RNA molecule of EGFR. Even more preferably, a nucleic acid molecule that modulates EGFR replication or expression comprises between 14 and 24 bases complementary to a RNA molecule of EGFR.

The invention provides a method for producing a class of nucleic acid-based gene modulating agents which exhibit a high degree of specificity for the RNA of a desired target. For example, the enzymatic nucleic acid molecule is preferably targeted to a highly conserved sequence region of target RNAs encoding EGFR (specifically EGFR genes) such that specific treatment of a disease or condition can be provided with either one or several nucleic acid molecules of the invention. Such nucleic acid molecules can be delivered exogenously to specific tissue or cellular targets as required. Alternatively, the nucleic acid molecules (e.g., ribozymes and antisense) can be expressed from DNA and/or RNA vectors that are delivered to specific cells.

As used in herein “cell” is used in its usual biological sense, and does not refer to an entire multicellular organism. The cell can, for example, be in vitro, e.g., in cell culture, or present in a multicellular organism, including,, e.g., birds, plants and mammals such as humans, cows, sheep, apes, monkeys, swine, dogs, and cats. The cell may be prokaryotic (e.g., bacterial cell) or eukaryotic (e.g., mammalian or plant cell).

By “EGFR proteins” is meant, protein receptor or a mutant protein derivative thereof, comprising epidermal growth factor receptor activity, for example binding of epidermal growth factor and/or tyrosine kinase activity.

By “highly conserved sequence region” is meant, a nucleotide sequence of one or more regions in a target gene does not vary significantly from one generation to the other or from one biological system to the other.

Nucleic acid-based inhibitorsof EGFR expression are useful for the prevention and/or treatment of cancers and cancerous conditions such as breast, lung, prostate, colorectal, brain, esophageal, bladder, pancreatic, cervical, head and neck, and ovarian cancer, melanoma, lymphoma, glioma, multidrug resistant cancers, and any other diseases or conditions that are related to or will respond to the levels of EGFR in a cell or tissue, alone or in combination with other therapies.

By “related” is meant that the reduction of EGFR expression (specifically EGFR gene) RNA levels and thus reduction in the level of the respective protein relieves, to some extent, the symptoms of the disease or condition.

The nucleic acid-based inhibitors of the invention are added directly, or can be complexed with cationic lipids, packaged within liposomes, or otherwise delivered to target cells or tissues. The nucleic acid or nucleic acid complexes can be locally administered to relevant tissues ex vivo, or in vivo through injection or infusion pump, with or without their incorporation in biopolymers. In preferred embodiments, the enzymatic nucleic acid inhibitors comprise sequences, which are complementary to the substrate sequences in Tables III to VIII and X. Examples of such enzymatic nucleic acid molecules also are shown in Tables III to VIII. Examples of such enzymatic nucleic acid molecules consist essentially of sequences defined in these tables.

In another embodiment, the invention features antisense nucleic acid molecules and 2-5A chimera including sequences complementary to the substrate sequences shown in Tables III to VIII and X. Such nucleic acid molecules can include sequences as shown for the binding arms of the enzymatic nucleic acid molecules in Tables III to VIII. Similarly, triplex molecules can be provided targeted to the corresponding DNA target regions, and containing the DNA equivalent of a target sequence or a sequence complementary to the specified target (substrate) sequence. Typically, antisense molecules are complementary to a target sequence along a single contiguous sequence of the antisense molecule. However, in certain embodiments, an antisense molecule can bind to substrate such that the substrate molecule forms a loop, and/or an antisense molecule can bind such that the antisense molecule forms a loop. Thus, the antisense molecule can be complementary to two (or even more) non-contiguous substrate sequences or two (or even more) non-contiguous sequence portions of an antisense molecule can be complementary to a target sequence or both.

By “consists essentially of” is meant that the active nucleic acid molecule of the invention, for example, an enzymatic nucleic acid molecule, contains an enzymatic center or core equivalent to those in the examples, and binding arms able to bind RNA such that cleavage at the target site occurs. Other sequences can be present which do not interfere with such cleavage. Thus, a core region can, for example, include one or more loop, stem-loop structure, or linker which does not prevent enzymatic activity. Thus, the underlined regions in the sequences in Tables III and IV can be such a loop, stem-loop, nucleotide linker, and/or non-nucleotide linker and can be represented generally as sequence “X”. For example, a core sequence for a hammerhead enzymatic nucleic acid can comprise a conserved sequence, such as 5′-CUGAUGAG-3′ and 5′-CGAA-3′ connected by “X”, where X is 5′-GCCGUUAGGC-3′ (SEQ ID NO 9632), or any other Stem II region known in the art, or a nucleotide and/or non-nucleotide linker. Similarly, for other nucleic acid molecules of the instant invention, such as Inozyme, G-cleaver, amberzyme, zinzyme, DNAzyme, antisense, 2-5A antisense, triplex forming nucleic acid, and decoy nucleic acids, other sequences or non-nucleotide linkers can be present that do not interfere with the function of the nucleic acid molecule.

Sequence X can be a linker of ≧2 nucleotides in length, preferably 3, 4, 5, 6, 7, 8, 9, 10, 15, 20, 26, 30, where the nucleotides can preferably be internally base-paired to form a stem of preferably ≧2 base pairs. Alternatively or in addition, sequence X can be a non-nucleotide linker. In yet another embodiment, the nucleotide linker X can be a nucleic acid aptamer, such as an ATP aptamer, HIV Rev aptamer (RRE), HIV Tat aptamer (TAR) and others (for a review see Gold et al., 1995, Annu. Rev. Biochem., 64, 763; and Szostak & Ellington, 1993, in The RNA World, ed. Gesteland and Atkins, pp. 511, CSH Laboratory Press). A “nucleic acid aptamer” as used herein is meant to indicate a nucleic acid sequence capable of interacting with a ligand. The 20.1 ligand can be any natural or a synthetic molecule, including but not limited to a resin, metabolites, nucleosides, nucleotides, drugs, toxins, transition state analogs, peptides, lipids, proteins, amino acids, nucleic acid molecules, hormones, carbohydrates, receptors, cells, viruses, bacteria and others.

In yet another embodiment, the non-nucleotide linker X is as defined herein. The term “non-nucleotide” as used herein include either abasic nucleotide, polyether, polyamine, polyamide, peptide, carbohydrate, lipid, or polyhydrocarbon compounds. Specific examples include those described by Seela and Kaiser, Nucleic Acids Res. 1990, 18:6353 and Nucleic Acids Res. 1987, 15:3113; Cload and Schepartz, J. Am. Chem. Soc. 1991, 113:6324; Richardson and Schepartz, J. Am. Chem. Soc. 1991, 113:5109; Ma et al., Nucleic Acids Res. 1993, 21:2585 and Biochemistry 1993, 32:1751; Durand et al., Nucleic Acids Res. 1990, 18:6353; McCurdy et al., Nucleosides & Nucleotides 1991, 10:287; Jschke et al., Tetrahedron Lett. 1993, 34:301; Ono et al., Biochemistry 1991, 30:9914; Arnold et al., International Publication No. WO 89/02439; Usman et al., International Publication No. WO 95/06731; Dudycz et al., International Publication No. WO 95/11910 and Ferentz and Verdine, J. Am. Chem. Soc. 1991, 113:4000, all hereby incorporated by reference herein. A “non-nucleotide” further means any group or compound which can be incorporated into a nucleic acid chain in the place of one or more nucleotide units, including either sugar and/or phosphate substitutions, and allows the remaining bases to exhibit their enzymatic activity. The group or compound can be abasic in that it does not contain a commonly recognized nucleotide base, such as adenosine, guanine, cytosine, uracil or thymine. Thus, in a preferred embodiment, the invention features an enzymatic nucleic acid molecule having one or more non-nucleotide moieties, and having enzymatic activity to cleave an RNA or DNA molecule.

In another aspect of the invention, enzymatic nucleic acid molecules or antisense molecules that interact with target RNA molecules and down-regulate EGFR (specifically EGFR gene) activity are expressed from transcription units inserted into DNA or RNA vectors. The recombinant vectors are preferably DNA plasmids or viral vectors. Enzymatic nucleic acid molecule or antisense expressing viral vectors can be constructed based on, but not limited to, adeno-associated virus, retrovirus, adenovirus, or alphavirus. Preferably, the recombinant vectors capable of expressing the enzymatic nucleic acid molecules or antisense are delivered as described above, and persist in target cells. Alternatively, viral vectors can be used that provide for transient expression of enzymatic nucleic acid molecules or antisense. Such vectors can be repeatedly administered as necessary. Once expressed, the enzymatic nucleic acid molecules or antisense bind to the target RNA and down-regulate its function or expression. Delivery of enzymatic nucleic acid molecule or antisense expressing vectors can be systemic, such as by intravenous or intramuscular administration, by administration to target cells ex-planted from the patient followed by reintroduction into the patient, or by any other means that would allow for introduction into the desired target cell. Antisense DNA can be expressed via the use of a single stranded DNA intracellular expression vector.

By “vectors” is meant any nucleic acid- and/or viral-based technique used to deliver a desired nucleic acid.

By “patient” is meant an organism, which is a donor or recipient of explanted cells or the cells themselves. “Patient” also refers to an organism to which the nucleic acid molecules of the invention can be administered. Preferably, a patient is a mammal or mammalian cells. More preferably, a patient is a human or human cells.

By “enhanced enzymatic activity” is meant to include activity measured in cells and/or in vivo where the activity is a reflection of both the catalytic activity and the stability of the nucleic acid molecules of the invention. In this invention, the product of these properties can be increased in vivo compared to an all RNA enzymatic nucleic acid or all DNA enzyme. In some cases, the activity or stability of the nucleic acid molecule can be decreased (i.e., less than ten-fold), but the overall activity of the nucleic acid molecule is enhanced, in vivo.

The nucleic acid molecules of the instant invention, individually, or in combination or in conjunction with other drugs, can be used to treat diseases or conditions discussed above. For example, to treat a disease or condition associated with the levels of EGFR, the patient can be treated, or other appropriate cells can be treated, as is evident to those skilled in the art, individually or in combination with one or more drugs under conditions suitable for the treatment.

In a further embodiment, the described molecules, such as antisense or ribozymes, can be used in combination with other known treatments to treat conditions or diseases discussed above. For example, the described molecules can be used in combination with one or more known therapeutic agents to treat breast, lung, prostate, colorectal, brain, esophageal, bladder, pancreatic, cervical, head and neck, and ovarian cancer, melanoma, lymphoma, glioma, multidrug resistant cancers, and/or other cancers which respond to the modulation of EGFR expression.

In another embodiment, the invention features nucleic acid-based inhibitors (e.g., enzymatic nucleic acid molecules (eg; ribozymes), antisense nucleic acids, 2-5A antisense chimeras, triplex DNA, antisense nucleic acids containing RNA cleaving chemical groups) and methods for their use to down regulate or inhibit the expression of genes (e.g., EGFR) capable of progression and/or maintenance of cancer, and/or other disease states which respond to the modulation of EGFR expression.

By “comprising” is meant including, but not limited to, whatever follows the word “comprising”. Thus, use of the term “comprising” indicates that the listed elements are required or mandatory, but that other elements are optional and may or may not be present. By “consisting of” is meant including, and limited to, whatever follows the phrase “consisting of”. Thus, the phrase “consisting of” indicates that the listed elements are required or mandatory, and that no other elements may be present.

Other features and advantages of the invention will be apparent from the following description of the preferred embodiments thereof, and from the claims.

### DESCRIPTION OF THE PREFERRED EMBODIMENTS

First the drawings will be described briefly.

Drawings

FIG. 1 shows the secondary structure model for seven different classes of enzymatic nucleic acid molecules. Arrow indicates the site of cleavage. - - - indicate the target sequence. Lines interspersed with dots are meant to indicate tertiary interactions. - - - is meant to indicate base-paired interaction. Group I Intron: PI-P9.0 represent various stem-loop structures (Cech et al., 1994, Nature Struc. Bio., 1, 273). RNase P (MIRNA): EGS represents external guide sequence (Forster et al., 1990, Science, 249, 783; Pace et al., 1990, J. Biol. Chem., 265, 3587). Group II Intron: 5′ SS means 5′ splice site; 3′ SS means 3′-splice site; IBS means intron binding site; EBS means exon binding site (Pyle et al., 1994, Biochemistry, 33, 2716). VS RNA: I-VI are meant to indicate six stem-loop structures; shaded regions are meant to indicate tertiary interaction (Collins, International PCT Publication No. WO 96/19577). HDV Ribozyme:: I-IV are meant to indicate four stem-loop structures (Been et al., U.S. Pat. No. 5,625,047). Hammerhead Ribozyme: I-III are meant to indicate three stem-loop structures; stems I-III can be of any length and can be symmetrical or asymmetrical (Usman et al., 1996, Curr. Op. Struct. Bio., 1, 527). Hairpin Ribozyme: Helix 1, 4 and 5 can be of any length; Helix 2 is between 3 and 8 base-pairs long; Y is a pyrimidine; Helix 2 (H2) is provided with a least 4 base pairs (i.e., n is 1, 2, 3 or 4) and helix 5 can be optionally provided of length 2 or more bases (preferably 3-20bases, i.e., m is from 1-20 or more). Helix 2 and helix 5 can be covalently linked by one or more bases (i.e., r is >1 base). Helix 1, 4 or 5 can also be extended by 2 or more base pairs (e.g., 4-20 base pairs) to stabilize the ribozyme structure, and preferably is a protein binding site. In each instance, each N and N′ independently is any normal or modified base and each dash represents a potential base-pairing interaction. These nucleotides can be modified at the sugar, base or phosphate. Complete base-pairing is not required in the helices, but is preferred. Helix 1 and 4 can be of any size (i.e., o and p is each independently from 0 to any number, e.g., 20) as long as some base-pairing is maintained. Essential bases are shown as specific bases in the structure, but those in the art will recognize that one or more can be modified chemically (abasic, base, sugar and/or phosphate modifications) or replaced with another base without significant effect. Helix 4 can be formed from two separate molecules, i.e., without a connecting loop. The connecting loop when present can be a ribonucleotide with or without modifications to its base, sugar or phosphate. “q”≧is 2 bases. The connecting loop can also be replaced with a non-nucleotide linker molecule. H refers to bases A, U, or C. Y refers to pyrimidine bases. “______” refers to a covalent bond. (Burke et al., 1996, Nucleic Acids & Mol. Biol., 10, 129; Chowrira et al., U.S. Pat. No. 5,631,359).

FIG. 2 shows examples of chemically stabilized ribozyme motifs. HH Rz, represents hammerhead ribozyme motif (Usman et al., 1996, Curr. Op. Struct. Bio., 1, 527); NCH Rz represents the NCH ribozyme motif (Ludwig & Sproat, International PCT Publication No. WO 98/58058); G-Cleaver, represents G-cleaver ribozyme motif (Kore et al., 1998, Nucleic Acids Research 26, 4116-4120, Eckstein et al., International PCT publication No. WO 99/16871). N or n, represent independently a nucleotide which can be same or different and have complementarity to each other; rI, represents ribo-Inosine nucleotide; arrow indicates the site of cleavage within the target. Position 4 of the HH Rz and the NCH Rz is shown as having 2′-C-allyl modification, but those skilled in the art will recognize that this position can be modified with other modifications well known in the art, so long as such modifications do not significantly inhibit the activity of the ribozyme.

FIG. 3 shows an example of the Amberzyme ribozyme motif that is chemically stabilized (see for example Beigelman et al., International PCT publication No. WO 99/55857).

FIG. 4 shows an example of the Zinzyme A ribozyme motif that is chemically stabilized (see for example Beigelman et al., Beigelman et al, International PCT publication No. WO 99/55857).

FIG. 5 shows an example of a DNAzyme motif described by Santoro et al., 1997, PNAS, 94,4262.

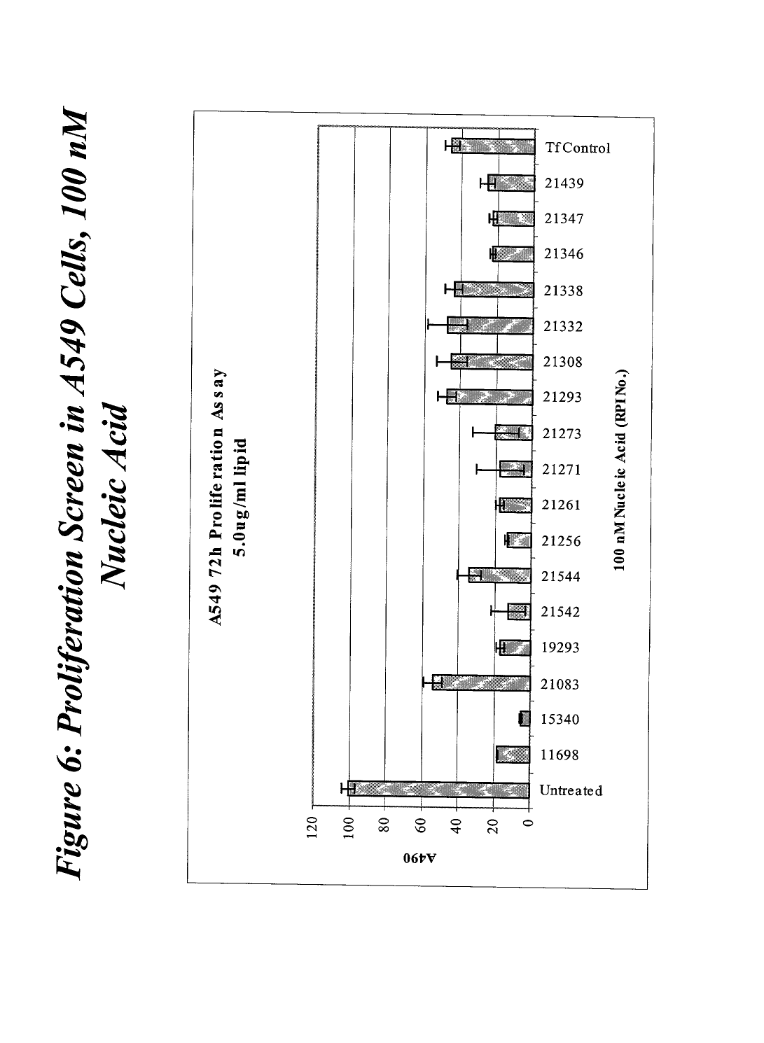

FIG. 6 is a graph showing nucleic acid (enzymatic nucleic acid and Genebloc) mediated anti-proliferation assay in A549 lung carcinoma cells. Cells were treated with 100 nm nucleic acid targeting EGFR RNA and a corresponding scrambled attenuated control or scrambled Genebloc control complexed with 5.0 μg/ml of lipid. Nucleic acid molecules and controls were compared to untreated cells after 24 hours post treatment.

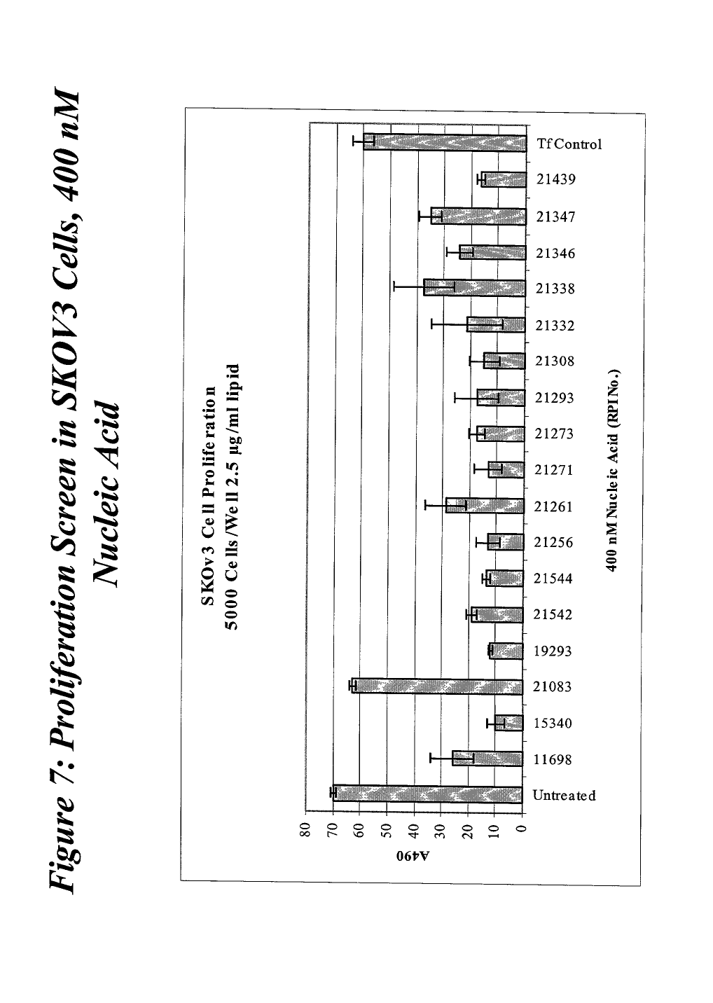

FIG. 7 is a graph showing nucleic acid (enzymatic nucleic acid and Genebloc) mediated anti-proliferation assay in SKOV3 ovarian carcinoma cells. Cells were treated with 400 nm nucleic acid targeting EGFR RNA and a corresponding scrambled attenuated control or scrambled Genebloc control complexed with 2.5 μg/ml of lipid. Nucleic acid molecules and controls were compared to untreated cells after 24 hours post treatment.

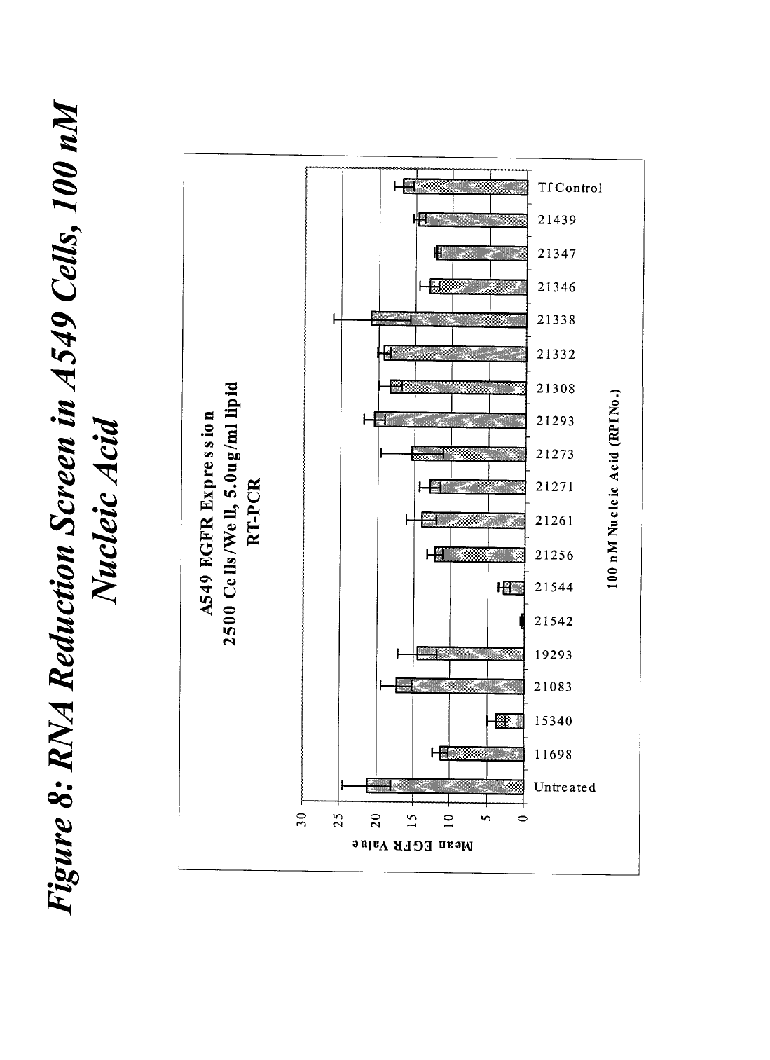

FIG. 8 is a graph which shows the reduction of EGFR RNA in A549 lung carcinoma cells treated with 100 nm nucleic acid targeting EGFR RNA and a corresponding scrambled attenuated control or scrambled Genebloc control complexed with 5.0 μg/ml of lipid. Nucleic acid molecules and controls were compared to untreated cells after 24 hours post treatment.

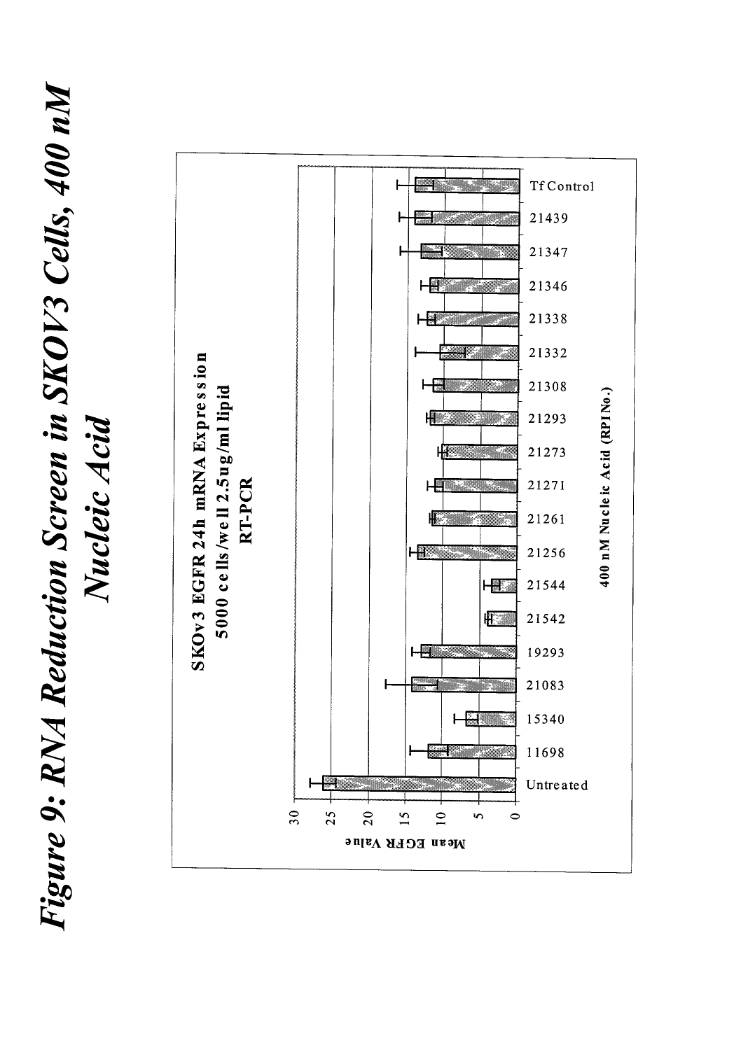

FIG. 9 is a graph which shows the reduction of EGFR RNA in SKOV3 ovarian carcinoma cells treated with 400 nm nucleic acid targeting EGFR RNA and a corresponding scrambled attenuated control or scrambled Genebloc control complexed with 2.5 μg/ml of lipid. Nucleic acid molecules and controls were compared to untreated cells after 24 hours post treatment.

### MECHANISM OF ACTION OF NUCLEIC ACID MOLECULES OF THE INVENTION

Antisense:

Antisense molecules can be modified or unmodified RNA, DNA, or mixed polymer oligonucleotides and primarily function by specifically binding to matching sequences resulting in inhibition of peptide synthesis (Wu-Pong, Nov 1994, BioPharm, 20-33). The antisense oligonucleotide binds to target RNA by Watson Crick base-pairing and blocks gene expression by preventing ribosomal translation of the bound sequences either by steric blocking or by activating RNase H enzyme. Antisense molecules can also alter protein synthesis by interfering with RNA processing or transport from the nucleus into the cytoplasm (Mukhopadhyay & Roth, 1996, Crit. Rev. in Oncogenesis 7, 151-190).

In addition, binding of single stranded DNA to RNA can result in nuclease degradation of m52 the heteroduplex (Wu-Pong, supra; Crooke, supra). To date, the only backbone modified DNA chemistry which act as substrates for RNase H are phosphorothioates, phosphorodithioates, and borontrifluoridates. Recently it has been reported that 2′-arabino and 2′-fluoro arabino-containing oligos can also activate RNase H activity.

A number of antisense molecules have been described that utilize novel configurations of chemically modified nucleotides, secondary structure, and/or RNase H substrate domains (Woolf et al., International PCT Publication No. WO 98/13526; Thompson et al., International PCT Publication No. WO 99/54459; Hartmann et al., USS No. 60/101,174 which was filed on Sep. 21, 1998) all of these are incorporated by reference herein in their entirety.

In addition, antisense deoxyoligoribonucleotides can be used to target RNA by means of DNA-RNA interactions, thereby activating RNase H, which digests the target RNA in the duplex. Antisense DNA can be expressed via the use of a single stranded DNA intracellular expression vector or equivalents and variations thereof.

Triplex Forming Oligonucleotides (TFO):

Single stranded DNA can be designed to bind to genomic DNA in a sequence specific manner. TFOs are comprised of pyrimidine-rich oligonucleotides which bind DNA helices through Hoogsteen Base-pairing (Wu-Pong, supra). The resulting triple helix composed of the DNA sense, DNA antisense, and TFO disrupts RNA synthesis by RNA polymerase. The TFO mechanism can result in gene expression or cell death since binding can be irreversible (Mukhopadhyay & Roth, supra).

2-5A Antisense Chimera:

The 2-5A system is an interferon mediated mechanism for RNA degradation found in higher vertebrates (Mitra et al., 1996, Proc Nat Acad Sci USA 93, 6780-6785). Two types of enzymes, 2-5A synthetase and RNase L, are required for RNA cleavage. The 2-5A synthetases require double stranded RNA to form 2′-5′ oligoadenylates (2-5A). 2-5A then acts as an allosteric effector for utilizing RNase L which has the ability to cleave single stranded RNA. The ability to form 2-5A structures with double stranded RNA makes this system particularly useful for inhibition of viral replication.

(2′-5′) oligoadenylate structures can be covalently linked to antisense molecules to form chimeric oligonucleotides capable of RNA cleavage (Torrence, supra). These molecules putatively bind and activate a 2-5A dependent RNase, the oligonucleotide/enzyme complex then binds to a target RNA molecule which can then be cleaved by the RNase enzyme.

Enzymatic Nucleic Acid:

Several varieties of naturally-occurring enzymatic RNAs are presently known. In addition, several in vitro selection (evolution) strategies (Orgel, 1979, Proc. R. Soc. London, B 205, 435) have been used to evolve new nucleic acid catalysts capable of catalyzing cleavage and ligation of phosphodiester linkages (Joyce, 1989, Gene, 82, 83-87; Beaudry et al., 1992, Science 257, 635-641; Joyce, 1992, Scientific American 267, 90-97; Breaker et al., 1994, TIBTECH 12, 268; Bartel et al.,1993, Science 261:1411-1418; Szostak, 1993, TIBS 17, 89-93; Kumar et al., 1995, FASEB J., 9, 1183; Breaker, 1996, Curr. Op. Biotech., 7, 442; Santoro et al., 1997, Proc. Natl. Acad. Sci., 94, 4262; Tang et al., 1997, RNA 3, 914; Nakamaye & Eckstein, 1994, supra; Long & Uhlenbeck, 1994, supra; Ishizaka et al., 1995, supra; Vaish et al., 1997, Biochemistry 36, 6495; all of these are incorporated by reference herein). Each can catalyze a series of reactions including the hydrolysis of phosphodiester bonds in trans (and thus can cleave other RNA molecules) under physiological conditions.

Nucleic acid molecules of this invention will block to some extent EGFR and/or HER2 protein expression and can be used to treat disease or diagnose disease associated with the levels of EGFR and/or HER2. Enzymatic nucleic acid sequences targeting EGFR RNA and sequences that can be targeted with nucleic acid molecules of the invention to down-regulate EGFR expression are shown in Tables III-XIII. Sequences that can be targeted with enzymatic nucleic acid molecules of the invention to down-regulate EGFR and/or HER2 expression are shown in Table X.

The enzymatic nature of an enzymatic nucleic acid molecule has significant advantages, one advantage being that the concentration of enzymatic nucleic acid molecule necessary to affect a therapeutic treatment is lower. This advantage reflects the ability of the enzymatic nucleic acid molecule to act enzymatically. Thus, a single enzymatic nucleic acid molecule is able to cleave many molecules of target RNA. In addition, the enzymatic nucleic acid molecule is a highly specific inhibitor, with the specificity of inhibition depending not only on the base-pairing mechanism of binding to the target RNA, but also on the mechanism of target RNA cleavage. Single mismatches, or base-substitutions, near the site of cleavage can be chosen to completely eliminate catalytic activity of a enzymatic nucleic acid molecule. Nucleic acid molecules having an endonuclease enzymatic activity are able to repeatedly cleave other separate RNA molecules in a nucleotide base sequence-specific manner. Such enzymatic nucleic acid molecules can be targeted to virtually any RNA transcript, and achieved efficient cleavage in vitro (Zaug et al., 324, Nature 429 1986; Uhlenbeck, 1987 Nature 328, 596; Kim et al., 84 Proc. Natl. Acad. Sci. USA 8788, 1987; Dreyfus, 1988, Einstein Quart. J. Bio. Med., 6, 92; Haseloff and Gerlach, 334 Nature 585, 1988; Cech, 260 JAMA 3030, 1988; and Jefferies et al., 17 Nucleic Acids Research 1371, 1989; Santoro et al., 1997 supra).

Because of their sequence specificity, trans-cleaving enzymatic nucleic acid molecules can be used as therapeutic agents for human disease (Usman & McSwiggen, 1995 Ann. Rep. Med. Chem. 30, 285-294; Christoffersen and Marr, 1995 J. Med. Chem. 38, 2023-2037). Enzymatic nucleic acid molecules can be designed to cleave specific RNA targets within the background of cellular RNA. Such a cleavage event renders the RNA non-functional and abrogates protein expression from that RNA. In this manner, synthesis of a protein associated with a disease state can be selectively inhibited (Warashina et al., 1999, Chemistry and Biology, 6, 237-250).

Enzymatic nucleic acid molecules of the invention that are allosterically regulated (“allozymes”) can be used to down-regulate EGFR expression. These allosteric enzymatic nucleic acids or allozymes (see for example George et al., U.S. Pat. Nos. 5,834,186 and 5,741,679, Shih et al., U.S. Pat. No. 5,589,332, Nathan et al., U.S. Pat. No. 5,871,914, Nathan and Ellington, International PCT publication No. WO 00/24931, Breaker et al., International PCT Publication Nos. WO 00/26226 and 98/27104, and Sullenger et al., International PCT publication No. WO 99/29842) are designed to respond to a signaling agent, for example, mutant EGFR protein, wild-type EGFR protein, mutant EGFR RNA, wild-type EGFR RNA, other proteins and/or RNAs involved in EGFR signal transduction, compounds, metals, polymers, molecules and/or drugs that are targeted to EGFR expressing cells etc., which in turn modulates the activity of the enzymatic nucleic acid molecule. In response to interaction with a predetermined signaling agent, the allosteric enzymatic nucleic acid molecule's activity is activated or inhibited such that the expression of a particular target is selectively down-regulated.

The target can comprise wild-type EGFR, mutant EGFR, and/or a predetermined component of the EGFR signal transduction pathway. In a specific example, allosteric enzymatic nucleic acid molecules that are activated by interaction with a RNA encoding a mutant EGFR protein are used as therapeutic agents in vivo. The presence of RNA encoding the mutant EGFR protein activates the allosteric enzymatic nucleic acid molecule that subsequently cleaves the RNA encoding a mutant EGFR protein resulting in the inhibition of mutant EGFR protein expression. In this manner, cancerous cells that express the mutant form of the EGFR protein are selectively targeted.

In another non-limiting example, an allozyme can be activated by a EGFR protein, peptide, or mutant polypeptide that caused the allozyme to inhibit the expression of EGFR gene, by, for example, cleaving RNA encoded by EGFR gene. In this non-limiting example, the allozyme acts as a decoy to inhibit the flnction of EGFR and also inhibit the expression of EGFR once activated by the EGFR protein. The nucleic acid molecules of the instant invention are also referred to as GeneBloc reagents, which are essentially nucleic acid molecules (eg; ribozymes, antisense) capable of down-regulating gene expression.

Target Sites

Targets for useful enzymatic nucleic acid molecules and antisense nucleic acids can be determined as disclosed in Draper et al., WO 93/23569; Sullivan et al., WO 93/23057;Thompson et al., WO 94/02595; Draper et al., WO 95/04818; McSwiggen et al., U.S. Pat. No. 5,525,468, and hereby incorporated by reference herein in totality. Other examples include the following PCT applications, which concern inactivation of expression of disease-related genes: WO 95/23225, WO 95/13380, WO 94/02595, incorporated by reference herein. Rather than repeat the guidance provided in those documents here, below are provided specific examples of such methods, not limiting to those in the art. Enzymatic nucleic acid molecules and antisense to such targets are designed as described in those applications and synthesized to be tested in vitro and in vivo, as also described. The sequences of human EGFR RNAs were screened for optimal enzymatic nucleic acid and antisense target sites using a computer-folding algorithm. Antisense, hammerhead, DNAzyme, NCH, amberzyme, zinzyme, or G-Cleaver enzymatic nucleic acid molecule binding/cleavage sites were identified. These sites are shown in Tables III to VIII and X (all sequences are 5′ to 3′ in the tables; underlined regions can be any sequence “X” or linker X, the actual sequence is not relevant here). The nucleotide base position is noted in the Tables as that site to be cleaved by the designated type of enzymatic nucleic acid molecule. While human sequences can be screened and enzymatic nucleic acid molecule and/or antisense thereafter designed, as discussed in Stinchcomb et al., WO 95/23225, mouse targeted enzymatic nucleic acid molecules can be useful to test efficacy of action of the enzymatic nucleic acid molecule and/or antisense prior to testing in humans.

Antisense, hammerhead, DNAzyme, NCH, amberzyme, zinzyme or G-Cleaver enzymatic nucleic acid molecule binding/cleavage sites were identified. The nucleic acid molecules are individually analyzed by computer folding (Jaeger et al., 1989 Proc. Natl. Acad. Sci. USA, 86, 7706) to assess whether the sequences fold into the appropriate secondary structure. Those nucleic acid molecules with unfavorable intramolecular interactions such as between the binding arms and the catalytic core are eliminated from consideration. Varying binding arm lengths can be chosen to optimize activity.

Antisense, hammerhead, DNAzyme, NCH, amberzyme, zinzyme or G-Cleaver enzymatic nucleic acid molecule binding/cleavage sites were identified and were designed to anneal to various sites in the RNA target. The binding arms are complementary to the target site sequences described above. The nucleic acid molecules were chemically synthesized. The method of synthesis used follows the procedure for normal DNA/RNA synthesis as described below and in Usman et al., 1987 J. Am. Chem. Soc., 109, 7845; Scaringe et al., 1990 Nucleic Acids Res., 18, 5433; and Wincott et al., 1995 Nucleic Acids Res. 23, 2677-2684; Caruthers et al., 1992, Methods in Enzymology 211,3-19.

Synthesis of Nucleic acid Molecules

Synthesis of nucleic acids greater than 100 nucleotides in length is difficult using automated methods, and the therapeutic cost of such molecules is prohibitive. In this invention, small nucleic acid motifs (“small refers to nucleic acid motifs less than about 100 nucleotides in length, preferably less than about 80 nucleotides in length, and more preferably less than about 50 nucleotides in length; e.g., antisense oligonucleotides, hammerhead or the NCH ribozymes) are preferably used for exogenous delivery. The simple structure of these molecules increases the ability of the nucleic acid to invade targeted regions of RNA structure. Exemplary molecules of the instant invention are chemically synthesized, and others can similarly be synthesized.

Oligonucleotides (eg; antisense GeneBlocs) are synthesized using protocols known in the art as described in Caruthers et al., 1992, Methods in Enzymology 211, 3-19, Thompson et al., International PCT Publication No. WO 99/54459, Wincott et al., 1995, Nucleic Acids Res. 23, 2677-2684, Wincott et al., 1997, Methods Mol. Bio., 74, 59, Brennan et al., 1998, Biotechnol Bioeng., 61, 33-45, and Brennan, U.S. Pat. No. 6,001,311. All of these references are incorporated herein by reference. The synthesis of oligonucleotides makes use of common nucleic acid protecting and coupling groups, such as dimethoxytrityl at the 5′-end, and phosphoramidites at the 3′-end. In a non-limiting example, small scale syntheses are conducted on a 394 Applied Biosystems, Inc. synthesizer using a 0.2 μmol scale protocol with a 2.5 min coupling step for 2′-O-methylated nucleotides and a 45 sec coupling step for 2′-deoxy nucleotides. Table II outlines the amounts and the contact times of the reagents used in the synthesis cycle. Alternatively, syntheses at the 0.2 μmol scale can be performed on a 96-well plate synthesizer, such as the instrument produced by Protogene (Palo Alto, Calif.) with minimal modification to the cycle. A 33-fold excess (60 μL of 0.11 M=6.6 limol) of 2′-O-methyl phosphoramidite and a 105-fold excess of S-ethyl tetrazole (60 μL of 0.25 M=15 tmol) can be used in each coupling cycle of 2′-O-methyl residues relative to polymer-bound 5′-hydroxyl. A 22-fold excess (40 μL of 0.11 M=4.4 tmol) of deoxy phosphoramidite and a 70-fold excess of S-ethyl tetrazole (40 μL of 0.25 M=10 tmol) can be used in each coupling cycle of deoxy residues relative to polymer-bound 5′-hydroxyl. Average coupling yields on the 394 Applied Biosystems, Inc. synthesizer, determined by colorimetric quantitation of the trityl fractions, are typically 97.5-99%. Other oligonucleotide synthesis reagents for the 394 Applied Biosystems, Inc. synthesizer include; detritylation solution is 3% TCA in methylene chloride (ABI); capping is performed with 16% N-methyl imidazole in THF (ABI) and 10% acetic anhydride/10% 2,6-lutidine in THF (ABI); and oxidation solution is 16.9 mM 12, 49 mM pyridine, 9% water in THF (PERSEPTIVE™). Burdick & Jackson Synthesis Grade acetonitrile is used directly from the reagent bottle. S-Ethyltetrazole solution (0.25 M in acetonitrile) is made up from the solid obtained from American International Chemical, Inc. Alternately, for the introduction of phosphorothioate linkages, Beaucage reagent (3H-1,2-Benzodithiol-3-one 1,1-dioxide, 0.05 M in acetonitrile) is used.

Deprotection of the antisense oligonucleotides is performed as follows: the polymer-bound trityl-on oligoribonucleotide is transferred to a 4 mL glass screw top vial and suspended in a solution of 40% aq. methylamine (1 mL) at 65° C. for 10 min. After cooling to −20° C., the supernatant is removed from the polymer support. The support is washed three times with 1.0 mL of EtOH:MeCN:H20/3:1:1, vortexed and the supernatant is then added to the first supernatant. The combined supernatants, containing the oligoribonucleotide, are dried to a white powder.

The method of synthesis used for normal RNA including certain enzymatic nucleic acid molecules follows the procedure as described in Usman et al., 1987, J. Am. Chem. Soc., 109, 7845; Scaringe et al., 1990, Nucleic Acids Res., 18, 5433; and Wincott et al., 1995, Nucleic Acids Res. 23, 2677-2684 Wincott et al., 1997, Methods Mol. Bio., 74, 59, and makes use of common nucleic acid protecting and coupling groups, such as dimethoxytrityl at the 5′-end, and phosphoramidites at the 3′-end. In a non-limiting example, small scale syntheses are conducted on a 394 Applied Biosystems, Inc. synthesizer using a 0.2 lmol scale protocol with a 7.5 min coupling step for alkylsilyl protected nucleotides and a 2.5 min coupling step for 2′-O-methylated nucleotides. Table II outlines the amounts and the contact times of the reagents used in the synthesis cycle. Alternatively, syntheses at the 0.2 μmol scale can be done on a 96-well plate synthesizer, such as the instrument produced by Protogene (Palo Alto, Calif.) with minimal modification to the cycle. A 33-fold excess (60 μL of 0.11 M=6.6 tmol) of 2′-O-methyl phosphoramidite and a 75-fold excess of S-ethyl tetrazole (60 μL of 0.25 M=15 μmol) can be used in each coupling cycle of 2′-O-methyl residues relative to polymer-bound 5′-hydroxyl. A 66-fold excess (120 μL of 0.11 M=13.2 μmol) of alkylsilyl (ribo) protected phosphoramidite and a 150-fold excess of S-ethyl tetrazole (120 μL of 0.25 M=30 μmol) can be used in each coupling cycle of ribo residues relative to polymer-bound 5′-hydroxyl. Average coupling yields on the 394 Applied Biosystems, Inc. synthesizer, determined by calorimetric quantitation of the trityl fractions, are typically 97.5-99%. Other oligonucleotide synthesis reagents for the 394 Applied Biosystems, Inc. synthesizer include; detritylation solution is 3% TCA in methylene chloride (ABI); capping is performed with 16% N-methyl imidazole in THF (ABI) and 10% acetic anhydride/10% 2,6-lutidine in THF (ABI); oxidation solution is 16.9 mM I₂, 49 mM pyridine, 9% water in THF (PERSEPTIVETM). Burdick & Jackson Synthesis Grade acetonitrile is used directly from the reagent bottle. S-Ethyltetrazole solution (0.25 M in acetonitrile) is made up from the solid obtained from American International Chemical, Inc. Alternately, for the introduction of phosphorothioate linkages, Beaucage reagent (3H-1,2-Benzodithiol-3-one 1,1-dioxide 0.05 M in acetonitrile) is used.

Deprotection of the RNA is performed using either a two-pot or one-pot protocol. For the two-pot protocol, the polymer-bound trityl-on oligoribonucleotide is transferred to a 4 mL glass screw top vial and suspended in a solution of 40% aq. methylamine (1 mL) at 65° C. for 10 min. After cooling to −20° C., the supernatant is removed from the polymer support. The support is washed three times with 1.0 mL of EtOH:MeCN:H2O/3:1:1, vortexed and the supernatant is then added to the first supernatant. The combined supernatants, containing the oligoribonucleotide, are dried to a white powder. The base deprotected oligoribonucleotide is resuspended in anhydrous TEA/HF/NMP solution (300 μL of a solution of 1.5 mL N-methylpyrrolidinone, 750 μL TEA and 1 mL TEA.3HF to provide a 1.4 M HF concentration) and heated to 65° C. After 1.5 h, the oligomer is quenched with 1.5 M NH₄HCO₃.

Alternatively, for the one-pot protocol, the polymer-bound trityl-on oligoribonucleotide is transferred to a 4 mL glass screw top vial and suspended in a solution of 33% ethanolic methylamine/DMSO: 1/1 (0.8 mL) at 65° C. for 15 min. The vial is brought to r.t. TEA.3HF (0.1 mL) is added and the vial is heated at 65° C. for 15 min. The sample is cooled at −20° C. and then quenched with 1.5 M NH₄HCO₃.

For purification of the trityl-on oligomers, the quenched NH₄HCO₃ solution is loaded onto a C-18 containing cartridge that had been prewashed with acetonitrile followed by 50 mM TEAA. After washing the loaded cartridge with water, the RNA is detritylated with 0.5% TFA for 13 min. The cartridge is then washed again with water, salt exchanged with 1 M NaCl and washed with water again. The oligonucleotide is then eluted with 30% acetonitrile.

Inactive hammerhead ribozymes or binding attenuated control (BAC) oligonucleotides) are synthesized by substituting a U for G₅ and a U for A14 (numbering from Hertel, K. J., et al., 1992, Nucleic Acids Res., 20, 3252). Similarly, one or more nucleotide substitutions can be introduced in other enzymatic nucleic acid molecules to inactivate the molecule and such molecules can serve as a negative control.

The average stepwise coupling yields are typically >98% (Wincott et al., 1995 Nucleic Acids Res. 23, 2677-2684). Those of ordinary skill in the art will recognize that the scale of synthesis can be adapted to be larger or smaller than the example described above including but not limited to 96 well format, all that is important is the ratio of chemicals used in the reaction.

Alternatively, the nucleic acid molecules of the present invention can be synthesized separately and joined together post-synthetically, for example by ligation (Moore et al., 1992, Science 256, 9923; Draper et al., International PCT publication No. WO 93/23569; Shabarova et al., 1991, Nucleic Acids Research 19, 4247; Bellon et al., 1997, Nucleosides & Nucleotides, 16, 951; Bellon et al., 1997, Bioconjugate Chem. 8, 204).

The nucleic acid molecules of the present invention are modified extensively to enhance stability by modification with nuclease resistant groups, for example, 2′-amino, 2′-C-allyl, 2′-flouro, 2′-O-methyl, 2′-H (for a review see Usman and Cedergren, 1992, TIBS 17, 34; Usman et al., 1994, Nucleic Acids Symp. Ser. 31, 163). Ribozymes are purified by gel electrophoresis using general methods or are purified by high pressure liquid chromatography (HPLC; See Wincott et al., Supra, the totality of which is hereby incorporated herein by reference) and are re-suspended in water.

The sequences of the nucleic acid molecules, including enzymatic nucleic acid molecules and antisense, that are chemically synthesized, are shown in Table VIII. The sequences of the enzymatic nucleic acid and antisense constructs that are chemically synthesized, are complementary to the Substrate sequences shown in Table VIII. Those in the art will recognize that these sequences are representative only of many more such sequences where the enzymatic portion of the ribozyme (all but the binding arms) is altered to affect activity. The ribozyme and antisense construct sequences listed in Tables III to VIII can be formed of ribonucleotides or other nucleotides or non-nucleotides. Such enzymatic nucleic acid molecules with enzymatic activity are equivalent to the enzymatic nucleic acid molecules described specifically in the Tables.

Optimizing Activity of the Nucleic Acid Molecule of the Invention.

Chemically synthesizing nucleic acid molecules with modifications (base, sugar and/or phosphate) that prevent their degradation by serum ribonucleases can increase their potency (see e.g., Eckstein et al., International Publication No. WO 92/07065; Perrault et al., 1990 Nature 344, 565; Pieken et al., 1991, Science 253, 314; Usman and Cedergren, 1992, Trends in Biochem. Sci. 17, 334; Usman et al., International Publication No. WO 93/15187; and Rossi et al., International Publication No. WO 91/03162; Sproat, U.S. Pat. No. 5,334,711; and Burgin et al., supra; all of these describe various chemical modifications that can be made to the base, phosphate and/or sugar moieties of the nucleic acid molecules herein). Modifications which enhance their efficacy in cells, and removal of bases from nucleic acid molecules to shorten oligonucleotide synthesis times and reduce chemical requirements are desired. (All these publications are hereby incorporated by reference herein).

There are several examples in the art describing sugar, base and phosphate modifications that can be introduced into nucleic acid molecules with significant enhancement in their nuclease stability and efficacy. For example, oligonucleotides are modified to enhance stability and/or enhance biological activity by modification with nuclease resistant groups, for example, 2′-amino, 2′-C-allyl, 2′-flouro, 2′-O-methyl, 2′-H, nucleotide base modifications (for a review see Usman and Cedergren, 1992, TIBS. 17, 34; Usman et al., 1994, Nucleic Acids Symp. Ser. 31, 163; Burgin et al., 1996, Biochemistry , 35, 14090). Sugar modification of nucleic acid molecules have been extensively described in the art (see Eckstein et al., International Publication PCT No. WO 92/07065; Perrault et al. Nature, 1990, 344, 565-568; Pieken et al. Science, 1991, 253, 314-317; Usman and Cedergren, Trends in Biochem. Sci. , 1992, 17, 334-339; Usman et al. International Publication PCT No. WO 93/15187; Sproat, U.S. Pat. No. 5,334,711 and Beigelman et al., 1995, J. Biol. Chem., 270, 25702; Beigelman et al., International PCT publication No. WO 97/26270; Beigelman et al., U.S. Pat. No. 5,716,824; Usman et al., U.S. Pat. No. 5,627,053; Woolf et al, International PCT Publication No. WO 98/13526; Thompson et al., USS No. 60/082,404 which was filed on Apr. 20, 1998; Karpeisky et al., 1998, Tetrahedron Lett., 39, 1131; Earnshaw and Gait, 1998, Biopolymers (Nucleic acid Sciences), 48, 39-55; Verma and Eckstein, 1998, Annu. Rev. Biochem., 67, 99-134; and Burlina et al., 1997,Bioorg. Med. Chem., 5, 1999-2010; all of the references are hereby incorporated in their totality by reference herein). Such publications describe general methods and strategies to determine the location of incorporation of sugar, base and/or phosphate modifications and the like into ribozymes without inhibiting catalysis, and are incorporated by reference herein. In view of such teachings, similar modifications can be used as described herein to modify the nucleic acid molecules of the instant invention.

While chemical modification of oligonucleotide internucleotide linkages with phosphorothioate, phosphorothioate, and/or 5′-methylphosphonate linkages improves stability, too many of these modifications can cause some toxicity. Therefore when designing nucleic acid molecules the amount of these internucleotide linkages should be minimized. The reduction in the concentration of these linkages should lower toxicity resulting in increased efficacy and higher specificity of these molecules.

Nucleic acid molecules having chemical modifications that maintain or enhance activity are provided. Such nucleic acid is also generally more resistant to nucleases than unmodified nucleic acid. Thus, in a cell and/or in vivo the activity may not be significantly lowered. Therapeutic nucleic acid molecules delivered exogenously are optimally stable within cells until translation of the target RNA has been inhibited long enough to reduce the levels of the undesirable protein. This period of time varies between hours to days depending upon the disease state. Clearly, nucleic acid molecules must be resistant to nucleases in order to function as effective intracellular therapeutic agents. Improvements in the chemical synthesis of RNA and DNA (Wincott et al., 1995 Nucleic Acids Res. 23, 2677; Caruthers et al., 1992, Methods in Enzymology 211,3-19 (incorporated by reference herein) have expanded the ability to modify nucleic acid molecules by introducing nucleotide modifications to enhance their nuclease stability as described above.

Use of the nucleic acid-based molecules of the invention can lead to better treatment of the disease progression by affording the possibility of combination therapies (e.g., multiple antisense or enzymatic nucleic acid molecules targeted to different genes, nucleic acid molecules coupled with known small molecule inhibitors, or intermittent treatment with combinations of molecules (including different motifs) and/or other chemical or biological molecules). The treatment of patients with nucleic acid molecules can also include combinations of different types of nucleic acid molecules.

Therapeutic nucleic acid molecules (e.g., enzymatic nucleic acid molecules and antisense nucleic acid molecules) delivered exogenously are optimally stable within cells until translation of the target RNA has been inhibited long enough to reduce the levels of the undesirable protein. This period of time varies between hours to days depending upon the disease state. These nucleic acid molecules should be resistant to nucleases in order to function as effective intracellular therapeutic agents. Improvements in the chemical synthesis of nucleic acid molecules described in the instant invention and in the art have expanded the ability to modify nucleic acid molecules by introducing nucleotide modifications to enhance their nuclease stability as described above.

In another embodiment, nucleic acid catalysts having chemical modifications that maintain or enhance enzymatic activity are provided. Such nucleic acids are also generally more resistant to nucleases than unmodified nucleic acid. Thus, in a cell and/or in vivo the activity of the nucleic acid may not be significantly lowered. As exemplified herein such enzymatic nucleic acids are useful in a cell and/or in vivo even if activity over all is reduced 10 fold (Burgin et al., 1996, Biochemistry, 35, 14090). Such enzymatic nucleic acids herein are said to “maintain” the enzymatic activity of an all RNA ribozyme or all DNA DNAzyme.

In another aspect the nucleic acid molecules comprise a 5′ and/or a 3′-cap structure.

By “cap structure” is meant chemical modifications, which have been incorporated at either terminus of the oligonucleotide (see for example Wincott et al., WO 97/26270, incorporated by reference herein). These terminal modifications protect the nucleic acid molecule from exonuclease degradation, and can help in delivery and/or localization within a cell. The cap can be present at the 5′-terminus (5′-cap) or at the 3′-terminus (3′-cap) or can be present on both terminus. In non-limiting examples, the 5′-cap includes inverted abasic residue (moiety), 4′,5′-methylene nucleotide; 1-(beta-D-erythrofuranosyl) nucleotide, 4′-thio nucleotide, carbocyclic nucleotide; 1,5-anhydrohexitol nucleotide; L-nucleotides; alpha-nucleotides; modified base nucleotide; phosphorodithioate linkage; threo-pentofuranosyl nucleotide; acyclic 3′,4′-seco nucleotide; acyclic 3,4-dihydroxybutyl nucleotide; acyclic 3,5-dihydroxypentyl nucleotide, 3′-3′-inverted nucleotide moiety; 3′-3′-inverted abasic moiety; 3′-2′-inverted nucleotide moiety; 3′-2′-inverted abasic moiety; 1,4-butanediol phosphate; 3′-phosphoramidate; hexylphosphate; aminohexyl phosphate; 3′-phosphate; 3′-phosphorothioate; phosphorodithioate; or bridging or non-bridging methylphosphonate moiety (for more details see Wincott et al., International PCT publication No. WO 97/26270, incorporated by reference herein).

In another embodiment the 3′-cap includes, for example 4′,5′-methylene nucleotide; 1-(beta-D-erythrofuranosyl) nucleotide; 4′-thio nucleotide, carbocyclic nucleotide; 5′-amino-alkyl phosphate; 1,3-diamino-2-propyl phosphate, 3-aminopropyl phosphate; 6-aminohexyl phosphate; 1,2-aminododecyl phosphate; hydroxypropyl phosphate; 1,5-anhydrohexitol nucleotide; L-nucleotide; alpha-nucleotide; modified base nucleotide; phosphorodithioate; threopentofuranosyl nucleotide; acyclic 3′,4′-seco nucleotide; 3,4-dihydroxybutyl nucleotide; 3,5-dihydroxypentyl nucleotide, 5′-5′-inverted nucleotide moiety; 5′-5′-inverted abasic moiety; 5′-phosphoramidate; 5′-phosphorothioate; 1,4-butanediol phosphate; 5′-amino; bridging and/or non-bridging 5′-phosphoramidate, phosphorothioate and/or phosphorodithioate, bridging or non bridging methylphosphonate and 5′-mercapto moieties (for more details see Beaucage and Iyer, 1993, Tetrahedron 49, 1925; incorporated by reference herein).

By the term “non-nucleotide” is meant any group or compound which can be incorporated into a nucleic acid chain in the place of one or more nucleotide units, including either sugar and/or phosphate substitutions, and allows the remaining bases to exhibit their enzymatic activity. The group or compound is abasic in that it does not contain a commonly recognized nucleotide base, such as adenosine, guanine, cytosine, uracil or thymine.

An “alkyl” group refers to a saturated aliphatic hydrocarbon, including straight-chain, branched-chain, and cyclic alkyl groups. Preferably, the alkyl group has 1 to 12 carbons. More preferably it is a lower alkyl of from 1 to 7 carbons, more preferably 1 to 4 carbons. The alkyl group can be substituted or unsubstituted. When substituted the substituted group(s) is preferably, hydroxyl, cyano, alkoxy, ═O, ═S, NO2 or N(CH3)2, amino, or SH. The term also includes alkenyl groups which are unsaturated hydrocarbon groups containing at least one carbon-carbon double bond, including straight-chain, branched-chain, and cyclic groups. Preferably, the alkenyl group has 1 to 12 carbons. More preferably it is a lower alkenyl of from 1 to 7 carbons, more preferably 1 to 4 carbons. The alkenyl group can be substituted or unsubstituted. When substituted the substituted group(s) is preferably, hydroxyl, cyano, alkoxy, ═O, ═S, NO2, halogen, N(CH3)2, amino, or SH. The term “alkyl” also includes alkynyl groups which have an unsaturated hydrocarbon group containing at least one carbon-carbon triple bond, including straight-chain, branched-chain, and cyclic groups. Preferably, the alkynyl group has 1 to 12 carbons. More preferably it is a lower alkynyl of from 1 to 7 carbons, more preferably 1 to 4 carbons. The alkynyl group can be substituted or unsubstituted. When substituted the substituted group(s) is preferably, hydroxyl, cyano, alkoxy, ═O, ═S, NO2 or N(CH3)2, amino or SH.

Such alkyl groups can also include aryl, alkylaryl, carbocyclic aryl, heterocyclic aryl, amide and ester groups. An “aryl” group refers to an aromatic group which has at least one ring having a conjugated p electron system and includes carbocyclic aryl, heterocyclic aryl and biaryl groups, all of which can be optionally substituted. The preferred substituent(s) of aryl groups are halogen, trihalomethyl, hydroxyl, SH, OH, cyano, alkoxy, alkyl, alkenyl, alkynyl, and amino groups. An “alkylaryl” group refers to an alkyl group (as described above) covalently joined to an aryl group (as described above). Carbocyclic aryl groups are groups wherein the ring atoms on the aromatic ring are all carbon atoms. The carbon atoms are optionally substituted. Heterocyclic aryl groups are groups having from 1 to 3 heteroatoms as ring atoms in the aromatic 18 ring and the remainder of the ring atoms are carbon atoms. Suitable heteroatoms include oxygen, sulfur, and nitrogen, and include furanyl, thienyl, pyridyl, pyrrolyl, N-lower alkyl pyrrolo, pyrimidyl, pyrazinyl, imidazolyl and the like, all optionally substituted. An “amide” refers to an —C(O)—NH—R, where R is either alkyl, aryl, alkylaryl or hydrogen. An “ester” refers to an —C(O)—OR′, where R is either alkyl, aryl, alkylaryl or hydrogen.

By “nucleotide” is meant a heterocyclic nitrogenous base in N-glycosidic linkage with a phosphorylated sugar. Nucleotides are recognized in the art to include natural bases (standard), and modified bases well known in the art. Such bases are generally located at the 1′ position of a nucleotide sugar moiety. Nucleotides generally comprise a base, sugar and a phosphate group. The nucleotides can be unmodified or modified at the sugar, phosphate and/or base moiety, (also referred to interchangeably as nucleotide analogs, modified nucleotides, non-natural nucleotides, non-standard nucleotides and other; see for example, Usman and McSwiggen, supra; Eckstein et al., International PCT Publication No. WO 92/07065; Usman et al., International PCT Publication No. WO 93/15187; Uhlman & Peyman, supra all are hereby incorporated by reference herein). There are several examples of modified nucleic acid bases known in the art as summarized by Limbach et al., 1994, Nucleic Acids Res. 22, 2183. Some of the non-limiting examples of chemically modified and other natural nucleic acid bases that can be introduced into nucleic acids include, for example, inosine, purine, pyridin-4-one, pyridin-2-one, phenyl, pseudouracil, 2, 4, 6-trimethoxy benzene, 3-methyl uracil, dihydrouridine, naphthyl, aminophenyl, 5-alkylcytidines (e.g., 5-methylcytidine), 5-alkyluridines (e.g., ribothymidine), 5-halouridine (e.g., 5-bromouridine) or 6-azapyrimidines or 6-alkylpyrimidines (e.g. 6-methyluridine), propyne, quesosine, 2-thiouridine, 4-thiouridine, wybutosine, wybutoxosine, 4-acetylcytidine, 5-(carboxyhydroxymethyl)uridine, 5 ′-carboxymethylaminomethyl-2-thiouridine, 5-carboxymethylaminomethyluridine, beta-D-galactosylqueosine, 1-methyladenosine, 1-methylinosine, 2,2-dimethylguanosine, 3-methylcytidine, 2-methyladenosine, 2-methylguanosine, N6-methyladenosine, 7-methylguanosine, 5-methoxyaminomethyl-2-thiouridine, 5-methylaminomethyluridine, 5-methylcarbonyhnethyluridine, 5-methyloxyuridine, 5-methyl-2-thiouridine, 2-methylthio-N6-isopentenyladenosine, beta-D-mannosylqueosine, uridine-5-oxyacetic acid, 2-thiocytidine, threonine derivatives and others (Burgin et al., 1996, Biochemistry, 35, 14090; Uhlman & Peyman, supra). By “modified bases” in this aspect is meant nucleotide bases other than adenine, guanine, cytosine and uracil at 1′ position or their equivalents; such bases can be used at any position, for example, within the catalytic core of an enzymatic nucleic acid molecule and/or in the substrate-binding regions of the nucleic acid molecule.

By “nucleoside” is meant a heterocyclic nitrogenous base in N-glycosidic linkage with a sugar. Nucleosides are recognized in the art to include natural bases (standard), and modified bases well known in the art. Such bases are generally located at the 1′ position of a nucleoside sugar moiety. Nucleosides generally comprise a base and sugar group. The nucleosides can be unmodified or modified at the sugar, and/or base moiety, (also referred to interchangeably as nucleoside analogs, modified nucleosides, non-natural nucleosides, non-standard nucleosides and other; see for example, Usman and McSwiggen, supra; Eckstein et al., International PCT Publication No. WO 92/07065; Usman et al., International PCT Publication No. WO 93/15187; Uhlman & Peyman, supra all are hereby incorporated by reference herein). There are several examples of modified nucleic acid bases known in the art as summarized by Limbach et al., 1994, Nucleic Acids Res. 22, 2183. Some of the non-limiting examples of chemically modified and other natural nucleic acid bases that can be introduced into nucleic acids include, inosine, purine, pyridin-4-one, pyridin-2-one, phenyl, pseudouracil, 2, 4, 6-trimethoxy benzene, 3-methyl uracil, dihydrouridine, naphthyl, aminophenyl, 5-alkylcytidines (e.g., 5-methylcytidine), 5-alkyluridines (e.g., ribothymidine), 5-halouridine (e.g., 5-bromouridine) or 6-azapyrimidines or 6-alkylpyrimidines (e.g. 6-methyluridine), propyne, quesosine, 2-thiouridine, 4-thiouridine, wybutosine, wybutoxosine, 4-acetylcytidine, 5-(carboxyhydroxymethyl)uridine, 5′-carboxyrnethylaminomethyl-2-thiouridine, 5-carboxymethylaminomethyluridine, □-D-galactosylqueosine, 1-methyladenosine, 1-methylinosine, 2,2-dimethylguanosine, 3-methylcytidine, 2-methyladenosine, 2-methylguanosine, N6-methyladenosine, 7-methylguanosine, 5-methoxyaminomethyl-2-thiouridine, 5-methylaminomethyluridine, 5-methylcarbonylmethyluridine, 5-methyloxyuridine, 5-methyl-2-thiouridine, 2-methylthio-N6-isopentenyladenosine, beta-D-mannosylqueosine, uridine-5-oxyacetic acid, 2-thiocytidine, threonine derivatives and others (Burgin et al., 1996, Biochemistry, 35, 14090; Uhlman & Peyman, supra). By “modified bases” in this aspect is meant nucleoside bases other than adenine, guanine, cytosine and uracil at 1′ position or their equivalents; such bases can be used at any position, for example, within the catalytic core of an enzymatic nucleic acid molecule and/or in the substrate-binding regions of the nucleic acid molecule.

In one embodiment, the invention features modified enzymatic nucleic acid molecules with phosphate backbone modifications comprising one or more phosphorothioate, phosphorodithioate, methylphosphonate, morpholino, amidate carbamate, carboxymethyl, acetamidate, polyamide, sulfonate, sulfonamide, sulfamate, formacetal, thioformacetal, and/or alkylsilyl, substitutions. For a review of oligonucleotide backbone modifications see Hunziker and Leumann, 1995, Nucleic Acid Analogues: Synthesis and Properties, in Modern Synthetic Methods, VCH, 331-417, and Mesmaeker et al., 1994, Novel Backbone Replacements for Oligonucleotides, in Carbohydrate Modifications in Antisense Research, ACS, 24-39. These references are hereby incorporated by reference herein.

By “abasic” is meant sugar moieties lacking a base or having other chemical groups in place of a base at the 1′ position, for example a 3′,3′-linked or 5′,5′-linked deoxyabasic ribose derivative (for more details see Wincott et al., International PCT publication No. WO 97/26270).

By “unmodified nucleoside” is meant one of the bases adenine, cytosine, guanine, thymine, uracil joined to the 1′ carbon of β-D-ribo-furanose.

By “modified nucleoside” is meant any nucleotide base which contains a modification in the chemical structure of an unmodified nucleotide base, sugar and/or phosphate.

In connection with 2′-modified nucleotides as described for the present invention, by “amino” is meant 2′-NH₂ or 2′-O—NH₂, which can be modified or unmodified. Such modified groups are described, for example, in Eckstein et al., U.S. Pat. No. 5,672,695 and Matulic-Adamic et al., WO 98/28317, respectively, which are both incorporated by reference in their entireties.

Various modifications to nucleic acid (e.g., antisense and ribozyme) structure can be made to enhance the utility of these molecules. For example, such modifications can enhance shelf-life, half-life in vitro, stability, and ease of introduction of such oligonucleotides to the target site, including e.g., enhancing penetration of cellular membranes and conferring the ability to recognize and bind to targeted cells.

Use of these molecules can lead to better treatment of the disease progression by affording the possibility of combination therapies (e.g., multiple enzymatic nucleic acid molecules targeted to different genes, enzymatic nucleic acid molecules coupled with known small molecule inhibitors, or intermittent treatment with combinations of enzymatic nucleic acid molecules (including different enzymatic nucleic acid molecule motifs) and/or other chemical or biological molecules). The treatment of patients with nucleic acid molecules can also include combinations of different types of nucleic acid molecules. Therapies can be devised which include a mixture of enzymatic nucleic acid molecules (including different enzymatic nucleic acid molecule motifs), antisense and/or 2-5A chimera molecules to one or more targets to alleviate symptoms of a disease.

Administration of Nucleic Acid Molecules

Methods for the delivery of nucleic acid molecules are described in Akhtar et al., 1992, Trends Cell Bio., 2, 139; and Delivery Strategies for Antisense Oligonucleotide Therapeutics, ed. Akhtar, 1995 which are both incorporated herein by reference. Sullivan et al., PCT WO 94/02595, further describes the general methods for delivery of enzymatic RNA molecules. These protocols can be utilized for the delivery of virtually any nucleic acid molecule. Nucleic acid molecules can be administered to cells by a variety of methods known to those familiar to the art, including, but not restricted to, encapsulation in liposomes, by iontophoresis, or by incorporation into other vehicles, such as hydrogels, cyclodextrins, biodegradable nanocapsules, and bioadhesive microspheres. Alternatively, the nucleic acid/vehicle combination is locally delivered by direct injection or by use of an infusion pump. Other routes of delivery include, but are not limited to oral (tablet or pill form) and/or intrathecal delivery (Gold, 1997, Neuroscience, 76, 1153-1158). Other approaches include the use of various transport and carrier systems, for example though the use of conjugates and biodegradable polymers. For a comprehensive review on drug delivery strategies including CNS delivery, see Ho et al., 1999, Curr. Opin. Mol. Ther., 1, 336-343 and Jain, Drug Delivery Systems: Technologies and Commercial Opportunities, Decision Resources, 1998 and Groothuis et al., 1997, J. NeuroVirol., 3, 387-400. More detailed descriptions of nucleic acid delivery and administration are provided in Sullivan et al., supra, Draper et al., PCT WO93/23569, Beigelman et al., PCT WO99/05094, and Klimuk et al., PCT WO99/04819 all of which have been incorporated by reference herein.

The molecules of the instant invention can be used as pharmaceutical agents. Pharmaceutical agents prevent, inhibit the occurrence, or treat (alleviate a symptom to some extent, preferably all of the symptoms) of a disease state in a patient.

The negatively charged polynucleotides of the invention can be administered (e.g., RNA, DNA or protein) and introduced into a patient by any standard means, with or without stabilizers, buffers, and the like, to form a pharmaceutical composition. When it is desired to use a liposome delivery mechanism, standard protocols for formation of liposomes can be followed. The compositions of the present invention can also be formulated and used as tablets, capsules or elixirs for oral administration; suppositories for rectal administration; sterile solutions; suspensions for injectable administration; and the other compositions known in the art.

The present invention also includes pharmaceutically acceptable formulations of the compounds described. These formulations include salts of the above compounds, e.g., acid addition salts, for example, salts of hydrochloric, hydrobromic, acetic acid, and benzene sulfonic acid.

A pharmacological composition or formulation refers to a composition or formulation in a form suitable for administration, e.g., systemic administration, into a cell or patient, preferably a human. Suitable forms, in part, depend upon the use or the route of entry, for example oral, transdermal, or by injection. Such forms should not prevent the composition or formulation from reaching a target cell (i.e., a cell to which the negatively charged polymer is desired to be delivered to). For example, pharmacological compositions injected into the blood stream should be soluble. Other factors are known in the art, and include considerations such as toxicity and forms which prevent the composition or formulation from exerting its effect.

By “systemic administration” is meant in vivo systemic absorption or accumulation of drugs in the blood stream followed by distribution throughout the entire body. Administration routes which lead to systemic absorption include, without limitations: intravenous, subcutaneous, intraperitoneal, inhalation, oral, intrapulmonary and intramuscular. Each of these administration routes expose the desired negatively charged polymers, e.g., nucleic acids, to an accessible diseased tissue. The rate of entry of a drug into the circulation has been shown to be a function of molecular weight or size. The use of a liposome or other drug carrier comprising the compounds of the instant invention can potentially localize the drug, for example, in certain tissue types, such as the tissues of the reticular endothelial system (RES). A liposome formulation which can facilitate the association of drug with the surface of cells, such as, lymphocytes and macrophages is also useful. This approach can provide enhanced delivery of the drug to target cells by taking advantage of the specificity of macrophage and lymphocyte immune recognition of abnormal cells, such as cancer cells.

By pharmaceutically acceptable formulation is meant, a composition or formulation that allows for the effective distribution of the nucleic acid molecules of the instant invention in the physical location most suitable for their desired activity. Non-limiting examples of agents suitable for formulation with the nucleic acid molecules of the instant invention include: PEG conjugated nucleic acids, phospholipid conjugated nucleic acids, nucleic acids containing lipophilic moieties, phosphorothioates, P-glycoprotein inhibitors (such as Pluronic P85) which can enhance entry of drugs into various tissues, for exaple the CNS (Jolliet-Riant and Tillement, 1999, Fundam. Clin. Pharmacol., 13, 16-26); biodegradable polymers, such as poly (DL-lactide-coglycolide) microspheres for sustained release delivery after implantation (Emerich, DF et al, 1999, Cell Transplant, 8, 47-58) Alkermes, Inc. Cambridge, Mass.; and loaded nanoparticles, such as those made of polybutylcyanoacrylate, which can deliver drugs across the blood brain barrier and can alter neuronal uptake mechanisms (Prog Neuropsychopharmacol Biol Psychiatry, 23, 941-949, 1999). Other non-limiting examples of delivery strategies, including CNS delivery of the nucleic acid molecules of the instant invention include material described in Boado et al., 1998, J. Pharm. Sci., 87, 1308-1315; Tyler et al, 1999, FEBS Lett., 421, 280-284; Pardridge et al., 1995, PNAS USA., 92, 5592-5596; Boado, 1995, Adv. Drug Delivery Rev., 15, 73-107; Aldrian-Herrada et al., 1998, Nucleic Acids Res., 26, 4910-4916; and Tyler et al., 1999, PNAS USA., 96, 7053-7058. All these references are hereby incorporated herein by reference.

The invention also features the use of the composition comprising surface-modified liposomes containing poly (ethylene glycol) lipids (PEG-modified, or long-circulating liposomes or stealth liposomes). Nucleic acid molecules of the invention can also comprise covalently attached PEG molecules of various molecular weights. These formulations offer a method for -increasing the accumulation of drugs in target tissues. This class of drug carriers resists opsonization and elimination by the mononuclear phagocytic system (MPS or RES), thereby enabling longer blood circulation times and enhanced tissue exposure for the encapsulated drug (Lasic et al. Chem. Rev. 1995, 95, 2601-2627; Ishiwata et al., Chem. Pharm. Bull. 1995, 43, 1005-1011). Such liposomes have been shown to accumulate selectively in tumors, presumably by extravasation and capture in the neovascularized target tissues (Lasic et al., Science 1995, 267, 1275-1276; Oku et al.,1995, Biochim. Biophys. Acta, 1238, 86-90). The long-circulating liposomes enhance the pharmacokinetics and pharmacodynamics of DNA and RNA, particularly compared to conventional cationic liposomes which are known to accumulate in tissues of the MPS (Liu et al., J. Biol. Chem. 1995, 42, 24864-24870; Choi et al., International PCT Publication No. WO 96/10391; Ansell et al., International PCT Publication No. WO 96/10390; Holland et al., International PCT Publication No. WO 96/10392; all of which are incorporated by reference herein). Long-circulating liposomes are also likely to protect drugs from nuclease degradation to a greater extent compared to cationic liposomes, based on their ability to avoid accumulation in metabolically aggressive MPS tissues such as the liver and spleen. All of these references are incorporated by reference herein.

The present invention also includes compositions prepared for storage or administration which include a pharmaceutically effective amount of the desired compounds in a pharmaceutically acceptable carrier or diluent. Acceptable carriers or diluents for therapeutic use are well known in the pharmaceutical art, and are described, for example, in Remington's Pharmaceutical Sciences, Mack Publishing Co. (A.R. Gennaro edit. 1985) hereby incorporated by reference herein. For example, preservatives, stabilizers, dyes and flavoring agents can be provided. These include sodium benzoate, sorbic acid and esters of p-hydroxybenzoic acid. In addition, antioxidants and suspending agents can be used.

A pharmaceutically effective dose is that dose required to prevent, inhibit the occurrence, or treat (alleviate a symptom to some extent, preferably all of the symptoms) of a disease state. The pharmaceutically effective dose depends on the type of disease, the composition used, the route of administration, the type of mammal being treated, the physical characteristics of the specific mammal under consideration, concurrent medication, and other factors which those skilled in the medical arts will recognize. Generally, an amount between 0.1 mg/kg and 100 mg/kg body weight/day of active ingredients is administered dependent upon potency of the negatively charged polymer.

The nucleic acid molecules of the invention and formulations thereof can be administered orally, topically, parenterally, by inhalation or spray or rectally in dosage unit formulations containing conventional non-toxic pharmaceutically acceptable carriers, adjuvants and vehicles. The term parenteral as used herein includes percutaneous, subcutaneous, intravascular (e.g., intravenous), intramuscular, or intrathecal injection or infusion techniques and the like. In addition, there is provided a pharmaceutical formulation comprising a nucleic acid molecule of the invention and a pharmaceutically acceptable carrier. One or more nucleic acid molecules of the invention can be present in association with one or more non-toxic pharmaceutically acceptable carriers and/or diluents and/or adjuvants, and if desired other active ingredients. The pharmaceutical compositions containing nucleic acid molecules of the invention can be in a form suitable for oral use, for example, as tablets, troches, lozenges, aqueous or oily suspensions, dispersible powders or granules, emulsion, hard or soft capsules, or syrups or elixirs.

Compositions intended for oral use can be prepared according to any method known to the art for the manufacture of pharmaceutical compositions and such compositions can contain one or more such sweetening agents, flavoring agents, coloring agents or preservative agents in order to provide pharmaceutically elegant and palatable preparations. Tablets contain the active ingredient in admixture with non-toxic pharmaceutically acceptable excipients that are suitable for the manufacture of tablets. These excipients can be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets can be uncoated or they can be coated by known techniques. In some cases such coatings can be prepared by known techniques to delay disintegration and absorption in the gastrointestinal tract and thereby provide a sustained action over a longer period. For example, a time delay material such as glyceryl monosterate or glyceryl distearate can be employed.

Formulations for oral use can also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin or olive oil.

Aqueous suspensions contain the active materials in admixture with excipients suitable for the manufacture of aqueous suspensions. Such excipients are suspending agents, for example sodium carboxymethylcellulose, methylcellulose, hydropropyl-methylcellulose, sodium alginate, polyvinylpyrrolidone, gum tragacanth and gum acacia; dispersing or wetting agents can be a naturally-occurring phosphatide, for example, lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions can also contain one or more preservatives, for example ethyl, or n-propyl p-hydroxybenzoate, one or more coloring agents, one or more flavoring agents, and one or more sweetening agents, such as sucrose or saccharin.

Oily suspensions can be formulated by suspending the active ingredients in a vegetable oil, for example arachis oil, olive oil, sesame oil or coconut oil, or in a mineral oil such as liquid paraffin. The oily suspensions can contain a thickening agent, for example beeswax, hard paraffin or cetyl alcohol. Sweetening agents and flavoring agents can be added to provide palatable oral preparations. These compositions can be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents or suspending agents are exemplified by those already mentioned above. Additional excipients, for example sweetening, flavoring and coloring agents, can also be present.

Pharmaceutical compositions of the invention can also be in the form of oil-in-water emulsions. The oily phase can be a vegetable oil or a mineral oil or mixtures of these. Suitable emulsifying agents can be naturally-occurring gums, for example gum acacia or gum tragacanth, naturally-occurring phosphatides, for example soy bean, lecithin, and esters or partial esters derived from fatty acids and hexitol, anhydrides, for example sorbitan monooleate, and condensation products of the said partial esters with ethylene oxide, for example polyoxyethylene sorbitan monooleate. The emulsions can also contain sweetening and flavoring agents.

Syrups and elixirs can be formulated with sweetening agents, for example glycerol, propylene glycol, sorbitol, glucose or sucrose. Such formulations can also contain a demulcent, a preservative and flavoring and coloring agents. The pharmaceutical compositions can be in the form of a sterile injectable aqueous or oleaginous suspension. This suspension can be formulated according to the known art using those suitable dispersing or wetting agents and suspending agents that have been mentioned above. The sterile injectable preparation can also be a sterile injectable solution or suspension in a non-toxic parentally acceptable diluent or solvent, for example as a solution in 1,3-butanediol. Among the acceptable vehicles and solvents that can be employed are water, Ringer's solution and isotonic sodium chloride solution. In addition, sterile, fixed oils are conventionally employed as a solvent or suspending medium. For this purpose any bland fixed oil can be employed including synthetic mono-or diglycerides. In addition, fatty acids such as oleic acid find use in the preparation of injectables.

The nucleic acid molecules of the invention can also be administered in the form of suppositories, e.g., for rectal administration of the drug. These compositions can be prepared by mixing the drug with a suitable non-irritating excipient that is solid at ordinary temperatures but liquid at the rectal temperature and will therefore melt in the rectum to release the drug. Such materials include cocoa butter and polyethylene glycols.

Nucleic acid molecules of the invention can be administered parenterally in a sterile medium. The drug, depending on the vehicle and concentration used, can either be suspended or dissolved in the vehicle. Advantageously, adjuvants such as local anesthetics, preservatives and buffering agents can be dissolved in the vehicle. Dosage levels of the order of from about 0.1 mg to about 140 mg per kilogram of body weight per day are useful in the treatment of the above-indicated conditions (about 0.5 mg to about 7 g per patient per day). The amount of active ingredient that can be combined with the carrier materials to produce a single dosage form varies depending upon the host treated and the particular mode of administration. Dosage unit forms generally contain between from about 1mg to about 500 mg of an active ingredient.

It is understood that the specific dose level for any particular patient depends upon a variety of factors including the activity of the specific compound employed, the age, body weight, general health, sex, diet, time of administration, route of administration, and rate of excretion, drug combination and the severity of the particular disease undergoing therapy. For administration to non-human animals, the composition can also be added to the animal feed or drinking water. It can be convenient to formulate the animal feed and drinking water compositions so that the animal takes in a therapeutically appropriate quantity of the composition along with its diet. It can also be convenient to present the composition as a premix for addition to the feed or drinking water.

The nucleic acid molecules of the present invention can also be administered to a patient in combination with other therapeutic compounds to increase the overall therapeutic effect. The use of multiple compounds to treat an indication can increase the beneficial effects while reducing the presence of side effects.

Alternatively, certain of the nucleic acid molecules of the instant invention can be expressed within cells from eukaryotic promoters (e.g., Izant and Weintraub, 1985, Science, 229, 345; McGarry and Lindquist, 1986, Proc. Natl. Acad. Sci., USA 83, 399; Scanlon et al., 1991, Proc. Natl. Acad. Sci. USA, 88, 10591-5; Kashani-Sabet et al., 1992, Antisense Res. Dev., 2, 3-15; Dropulic et al, 1992, J. Virol., 66, 1432-41; Weerasinghe et al., 1991, J. Virol., 65, 5531-4; Ojwang et al., 1992, Proc. Natl. Acad. Sci. USA, 89, 10802-6; Chen et al., 1992, Nucleic Acids Res., 20, 4581-9; Sarver et al., 1990 Science, 247, 1222-1225; Thompson et al., 1995, Nucleic Acids Res., 23, 2259; Good et al., 1997, Gene Therapy, 4, 45; all of these references are hereby incorporated in their totalities by reference herein). Those skilled in the art realize that any nucleic acid can be expressed in eukaryotic cells from the appropriate DNA/RNA vector. The activity of such nucleic acids can be augmented by their release from the primary transcript by a enzymatic nucleic acid (Draper et al, PCT WO 93/23569, and Sullivan et al., PCT WO 94/02595; Ohkawa et al., 1992, Nucleic Acids Symp. Ser., 27, 15-6; Taira et al., 1991, Nucleic Acids Res., 19, 5125-30; Ventura et al., 1993, Nucleic Acids Res., 21, 3249-55; Chowrira et al., 1994, J. Biol. Chem., 269, 25856; all of these references are hereby incorporated in their totalities by reference herein). Gene therapy approaches specific to the CNS are described by Blesch et al., 2000, Drug News Perspect., 13, 269-280; Peterson et al., 2000, Cent. Nerv. Syst. Dis., 485-508; Peel and Klein, 2000, J. Neurosci. Methods, 98, 95-104; Hagihara et al., 2000, Gene Ther., 7, 759-763; and Herrlinger et al., 2000, Methods Mol. Med., 35, 287-312. AAV-mediated delivery of nucleic acid to cells of the nervous system is further described by Kaplitt et al., U.S. Pat. No. 6,180,613.

In another aspect of the invention, RNA molecules of the present invention are preferably expressed from transcription units (see for example Couture et al., 1996, TIG., 12, 510) inserted into DNA or RNA vectors. The recombinant vectors are preferably DNA plasmids or viral vectors. Ribozyme expressing viral vectors can be constructed based on, but not limited to, adeno-associated virus, retrovirus, adenovirus, or alphavirus. Preferably, the recombinant vectors capable of expressing the nucleic acid molecules are delivered as described above, and persist in target cells. Alternatively, viral vectors can be used that provide for transient expression of nucleic acid molecules. Such vectors can be repeatedly administered as necessary. Once expressed, the nucleic acid molecule binds to the target mRNA. Delivery of nucleic acid molecule expressing vectors can be systemic, such as by intravenous or intramuscular administration, by administration to target cells ex-planted from the patient followed by reintroduction into the patient, or by any other means that would allow for introduction into the desired target cell (for a review see Couture et al., 1996, TIG., 12, 510).

In one aspect the invention features an expression vector comprising a nucleic acid sequence encoding at least one of the nucleic acid molecules of the instant invention is disclosed.

The nucleic acid sequence encoding the nucleic acid molecule of the instant invention is operable linked in a manner which allows expression of that nucleic acid molecule. In another aspect the invention features an expression vector comprising: a) a transcription initiation region (e.g., eukaryotic pol I, II or III initiation region); b) a transcription termination region (e.g., eukaryotic pol I, II or III termination region); c) a nucleic acid sequence encoding at least one of the nucleic acid catalyst of the instant invention; and wherein said sequence is operably linked to said initiation region and said termination region, in a manner which allows expression and/or delivery of said nucleic acid molecule. The vector can optionally include an open reading frame (ORF) for a protein operably linked on the 5′ side or the 3′-side of the sequence encoding the nucleic acid catalyst of the invention; and/or an intron (intervening sequences). Transcription of the nucleic acid molecule sequences are driven from a promoter for eukaryotic RNA polymerase I (pol I), RNA polymerase II (pol II), or RNA polymerase III (pol III). Transcripts from pol III or pol III promoters are expressed at high levels in all cells; the levels of a given pol II promoter in a given cell type depends on the nature of the gene regulatory sequences (enhancers, silencers, etc.) present nearby. Prokaryotic RNA polymerase promoters are also used, providing that the prokaryotic RNA polymerase enzyme is expressed in the appropriate cells (Elroy-Stein and Moss, 1990, Proc. Natl. Acad. Sci. USA, 87, 6743-7; Gao and Huang 1993, Nucleic Acids Res., 21, 2867-72; Lieber et al., 1993, Methods Enzymol., 217, 47-66; Zhou et al., 1990, Mol. Cell. Biol., 10, 4529-37). All of these references are incorporated by reference herein. Several investigators have demonstrated that nucleic acid molecules, such as ribozymes expressed from such promoters can function in mammalian cells (e.g. Kashani-Sabet et al., 1992, Antisense Res. Dev., 2, 3-15; Ojwang et al., 1992, Proc. Natl. Acad. Sci. U S A, 89, 10802-6; Chen et al., 1992, Nucleic Acids Res., 20, 4581-9; Yu et al., 1993, Proc. Natl. Acad. Sci. U S A, 90, 6340-4; L'Huillier et al., 1992, EMBO J., 11, 4411-8; Lisziewicz et al., 1993, Proc. Natl. Acad. Sci. U.S. A, 90, 8000-4; Thompson et al., 1995, Nucleic Acids Res., 23, 2259; Sullenger & Cech, 1993, Science, 262, 1566). More specifically, transcription units such as the ones derived from genes encoding U6 small nuclear (snRNA), transfer RNA (tRNA) and adenovirus VA RNA are useful in generating high concentrations of desired RNA molecules such as ribozymes in cells (Thompson et al., supra; Couture and Stinchcomb, 1996, supra; Noonberg et al., 1994, Nucleic Acid Res., 22, 2830; Noonberg et al., U.S. Pat. No. 5,624,803; Good et al., 1997, Gene Ther., 4, 45; Beigelman et al., International PCT Publication No. WO 96/18736; all of these publications are incorporated by reference herein. The above ribozyme transcription units can be incorporated into a variety of vectors for introduction into mammalian cells, including but not restricted to, plasmid DNA vectors, viral DNA vectors (such as adenovirus or adeno-associated virus vectors), or viral RNA vectors (such as retroviral or alphavirus vectors) (for a review see Couture and Stinchcomb, 1996, supra).

In another aspect the invention features an expression vector comprising nucleic acid sequence encoding at least one of the nucleic acid molecules of the invention, in a manner which allows expression of that nucleic acid molecule. The expression vector comprises in one embodiment; a) a transcription initiation region; b) a transcription termination region; c) a nucleic acid sequence encoding at least one said nucleic acid molecule; and wherein said sequence is operably linked to said initiation region and said termination region, in a manner which allows expression and/or delivery of said nucleic acid molecule.

In another embodiment the expression vector comprises: a) a transcription initiation region; b) a transcription termination region; c) an open reading frame; d) a nucleic acid sequence encoding at least one said nucleic acid molecule, wherein said sequence is operably linked to the 3′-end of said open reading frame; and wherein said sequence is operably linked to said initiation region, said open reading frame and said termination region, in a manner which allows expression and/or delivery of said nucleic acid molecule. In yet another embodiment the expression vector comprises: a) a transcription initiation region; b) a transcription termination region; c) an intron; d) a nucleic acid sequence encoding at least one said nucleic acid molecule; and wherein said sequence is operably linked to said initiation region, said intron and said termination region, in a manner which allows expression and/or delivery of said nucleic acid molecule.

In another embodiment, the expression vector comprises: a) a transcription initiation region; b) a transcription termination region; c) an intron; d) an open reading frame; e) a nucleic acid sequence encoding at least one said nucleic acid molecule, wherein said sequence is operably linked to the 3′-end of said open reading frame; and wherein said sequence is operably linked to said initiation region, said intron, said open reading frame and said termination region, in a manner which allows expression and/or delivery of said nucleic acid molecule.

### EXAMPLES

The following are non-limiting examples showing the selection, isolation, synthesis and activity of nucleic acids of the instant invention.

The following examples demonstrate the selection and design of Antisense, hammerhead, DNAzyme, NCH, Amberzyme, Zinzyme, or G-Cleaver ribozyme molecules and binding/cleavage sites within EGFR RNA.

### Example 1

### Identification of Potential Target Sites in Human EGFR RNA

The sequence of human EGFR genes are screened for accessible sites using a computer-folding algorithm. Regions of the RNA that do not form secondary folding structures and contained potential enzymatic nucleic acid molecule and/or antisense binding/cleavage sites are identified. The sequences of these binding/cleavage sites are shown in Tables III-VIII and X.

Sequences shown in Table X are RNA sequences that are homologous to both EGFR and HER2 genes (the nucleotide position shown is for the EGFR gene, Genbank accession No: NM_{—}005228).

### Example 2

### Selection of Enzymatic Nucleic Acid Cleavage Sites in Human EGFR RNA

Enzymatic nucleic acid molecule target sites are chosen by analyzing sequences of Human EGFR (Genbank accession No: NM_{—}005228) and prioritizing the sites on the basis of folding. Enzymatic nucleic acid molecules are designed that can bind each target and are individually analyzed by computer folding (Christoffersen et al., 1994 J. Mol. Struc. Theochem, 311, 273; Jaeger et al., 1989, Proc. Natl. Acad. Sci. USA, 86, 7706) to assess whether the enzymatic nucleic acid molecule sequences fold into the appropriate secondary structure. Those enzymatic nucleic acid molecules with unfavorable intramolecular interactions between the binding arms and the catalytic core are eliminated from consideration. As noted below, varying binding arm lengths can be chosen to optimize activity. Generally, at least 5 bases on each arm are able to bind to, or otherwise interact with, the target RNA.

### Example 3

### Chemical Synthesis and Purification of Ribozymes and Antisense for Efficient Cleavage and/or blocking of EGFR RNA

Enzymatic nucleic acid molecules and antisense constructs are designed to anneal to various sites in the RNA message. The binding arms of the enzymatic nucleic acid molecules are complementary to the target site sequences described above, while the antisense constructs are fully complementary to the target site sequences described above. The enzymatic nucleic acid molecules and antisense constructs were chemically synthesized. The method of synthesis used followed the procedure for normal RNA synthesis as described above and in Usman et al., (1987 J. Am. Chem. Soc., 109, 7845), Scaringe et al., (1990 Nucleic Acids Res., 18, 5433) and Wincott et al., supra, and made use of common nucleic acid protecting and coupling groups, such as dimethoxytrityl at the 5′-end, and phosphoramidites at the 3′-end. The average stepwise coupling yields were typically >98%.

Enzymatic nucleic acid molecules and antisense constructs are also synthesized from DNA templates using bacteriophage T7 RNA polymerase (Milligan and Uhlenbeck, 1989, Methods Enzymol. 180, 51). Enzymatic nucleic acid molecules and antisense constructs are purified by gel electrophoresis using general methods or are purified by high pressure liquid chromatography (HPLC; See Wincott et al., supra; the totality of which is hereby incorporated herein by reference) and are resuspended in water. The sequences of the chemically synthesized enzymatic nucleic acid molecules used in this study are shown below in Table VIII. The sequences of the chemically synthesized antisense constructs used in this study are complementary sequences to the Substrate sequences shown below as in Table III-VIII and X.

### Example 4

### Enzymatic Nucleic Acid Molecule Cleavage of EGFR RNA Target in vitro

Enzymatic nucleic acid molecules targeted to the human EGFR RNA are designed and synthesized as described above. These enzymatic nucleic acid molecules can be tested for cleavage activity in vitro, for example, using the following procedure. The target sequences and the nucleotide location within the EGFR RNA are given in Tables III-VIII. Target sequences and the nucleotide location within the EGFR RNA for sequences that can be used to target both EGFR and HER2 are shown in Table X.

Cleavage Reactions:

Full-length or partially full-length, internally-labeled target RNA for enzymatic nucleic acid molecule cleavage assay is prepared by in vitro transcription in the presence of [a-³²P] CTP, passed over a G 50 Sephadex column by spin chromatography and used as substrate RNA without further purification. Alternately, substrates are 5′-³²P-end labeled using T4 polynucleotide kinase enzyme. Assays are performed by pre-warming a 2× concentration of purified enzymatic nucleic acid molecule in enzymatic nucleic acid molecule cleavage buffer (50 mM Tris-HCl, pH 7.5 at 37° C., 10 mM MgCl₂) and the cleavage reaction was initiated by adding the 2× enzymatic nucleic acid molecule mix to an equal volume of substrate -.RNA (maximum of 1-5 nM) that was also pre-warmed in cleavage buffer. As an initial screen, assays are carried out for 1 hour at 37° C. using a final concentration of either 40 nM or 1 mM enzymatic nucleic acid molecule, i.e., enzymatic nucleic acid molecule excess. The reaction is quenched by the addition of an equal volume of 95% formamide, 20 mM EDTA, 0.05% bromophenol blue and 0.05% xylene cyanol after which the sample is heated to 95 C for 2 minutes, quick chilled and loaded onto a denaturing polyacrylamide gel. Substrate RNA and the specific RNA cleavage products generated by enzymatic nucleic acid molecule cleavage are visualized on an autoradiograph of the gel. The percentage of cleavage is determined by Phosphor Imager® quantitation of bands representing the intact substrate and the cleavage products.

### Example 5

### Nucleic acid down-regulation of EGFR target RNA in vivo

Nucleic acid molecules targeted to the human EGFR RNA are designed and synthesized as described above. These nucleic acid molecules can be tested for cleavage activity in vivo, for example using the procedures described below. The target sequences and the nucleotide location within the EGFR RNA are given in Tables III-VIII. Target sequences and the nucleotide location within the EGFR RNA for sequences that can be used to target both EGFR and HER2 are shown in Table X.

### Example 6

### In vivo Models Used to Evaluate the Down-Regulation of EGFR Gene Expression

A variety of endpoints have been used in cell culture models to evaluate EGFR-mediated effects after treatment with anti-EGFR agents. Phenotypic endpoints include inhibition of cell proliferation, apoptosis assays and reduction of EGJFR protein expression. Because overexpression of EGFR is directly associated with increased proliferation of tumor cells, a proliferation endpoint for cell culture assays is preferably used as a primary screen. There are several methods by which this endpoint can be measured. Following treatment of cells with nucleic acid molecules, cells are allowed to grow (typically 5 days) after which either the cell viability, the incorporation of [³H] thymidine into cellular DNA and/or the cell density can be measured. The assay of cell density is very straightforward and can be performed in a 96-well format using commercially available fluorescent nucleic acid stains (such as SytoOR 13 or CyQuant®). The assay using CyQuant® is described herein

As a secondary, confirmatory endpoint a nucleic acid-mediated decrease in the level of EGFR RNA and/or EGFR protein expression can be evaluated.

Validation of Cell Lines and Ribozyme Treatment Conditions

Two human cell lines (A549 and SKOV-3) that are known to express medium to high levels of EGFR protein are considered for nucleic acid screening. In order to validate these cell lines for EGFR-mediated sensitivity, both cell lines are treated with an EGFR specific antibody, for example mAB IMC-C225 (ImClone) and its effect on cell proliferation is determined. mAB is added to cells at concentrations ranging from 0-8 μM in medium containing either no serum (OptiMem), 0.1% or 0.5% FBS and efficacy is determined via cell proliferation. If maximal inhibition of proliferation (˜50%) in both cell lines is observed after addition of mAB at 0.5 nM in medium containing 0.1% or no FBS, then the fact that both cell lines are sensitive to an anti-EGFR agent (mAB) supports their use in experiments testing anti-EGFR nucleic acid molecules.

Prior to nucleic acid screening, the choice of the optimal lipid(s) and conditions for nucleic acid delivery is determined empirically for each cell line. Applicant has established a panel of cationic lipids (lipids as described in PCT application WO99/05094) that can be used to deliver nucleic acids to cultured cells and are very useful for cell proliferation assays that are typically 3-5 days in length. (Additional description of useful lipids is provided above, and those skilled in the art are also familiar with a variety of lipids that can be used for delivery of oligonucleotide to cells in culture.) Initially, this panel of lipid delivery vehicles is screened in A549 and SKOV-3 cells using previously established control oligonucleotides. Specific lipids and conditions for optimal delivery are selected for each cell line based on these screens. These conditions are used to deliver EGFR specific nucleic acids to cells for primary (inhibition of cell proliferation) and secondary (decrease in EGFR RNA/protein) efficacy endpoints.

Primary Screen: Inhibition of Cell Proliferation

Nucleic acid screens were performed using an automated, high throughput 96-well cell proliferation assay. Cell proliferation was measured over a 5-day treatment period using the nucleic acid stain CyQuant® for determining cell density. The growth of cells treated with enzymatic nucleic acid/lipid complexes were compared to both untreated cells and to cells treated with Scrambled-arm Attenuated core Controls (SAC). SACs can no longer bind to the target site due to the scrambled arm sequence and have nucleotide changes in the core that greatly diminish nucleic acid cleavage. These SACs are used to determine non-specific inhibition of cell growth caused by nucleic acid chemistry (i.e. multiple 2′ O-Me modified nucleotides, a single 2′C-allyl uridine, 4 phosphorothioates and a 3′ inverted abasic). The growth of cells treated with GeneBloc/lipid complexes were compared to both untreated cells and to cells treated with a scrambled control GeneBloc that can no longer bind to the target site due to the scrambled sequence. Lead nucleic acids are chosen from the primary screen based on their ability to inhibit cell proliferation in a specific manner. Dose response assays are carried out on these leads and a subset was advanced into a secondary screen using the level of EGFR protein as an endpoint.

Secondary Screen: Decrease in EGFR Protein and/or RNA

A secondary screen that measures the effect of anti-EGFR nucleic acids on EGFR protein and/or RNA levels is used to affirm preliminary findings. A EGFR ELISA for both A549 and SKOV-3 cells can been established and made available for use as an additional endpoint. In addition, a real time RT-PCR assay (TaqMan assay) has been developed to assess EGFR RNA reduction. Dose response activity of nucleic acid molecules of the instant invention can be used to assess both EGFR protein and RNA reduction endpoints.

Enzymatic Nucleic Acid Mechanism Assays

A TaqMan® assay for measuring the enzymatic nucleic acid-mediated decrease in EGFR RNA has been established. This assay is based on PCR technology and can measure in real time the production of EGFR mRNA relative to a standard cellular mRNA such as GAPDH. This RNA assay is used to establish proof that lead enzymatic nucleic acids are working through an RNA cleavage mechanism and result in a decrease in the level of EGFR mRNA, thus leading to a decrease in cell surface EGFR protein receptors and a subsequent decrease in tumor cell proliferation.

Animal Models

Evaluating the efficacy of anti-EGFR agents in animal models is an important prerequisite to human clinical trials. As in cell culture models, the most EGFR sensitive mouse tumor xenografts are those derived from human carcinoma cells that express high levels of EGFR protein. In a recent study, nude mice bearing human vulvar (A431), lung (A549 and SK-LC-16 NSCL and LX-1) and prostate (PC-3 and TSU-PRI) xenografts were sensitive to the anti-EGFR tyrosine kinase inhibitor ZD 1839 (Iressa), resulting in a partial regression of A431 tumor growth, 70-80% inhibition of tumor growth (A549, SKLC-16, TSU-PRI and PC-3 tumors), and 50-55% inhibition against the LX-1 tumor at a 150 mg kg dose (ip, every 3-4 days×4), (Sirotnak et al., 2000, Clin. Cancer Res., 6, 4885-48892). This same study compared the efficacy of ZD1839 alone or in combination with the commonly used chemotherapeutics, cisplatin, carboplatin, paclitaxel, docetaxel, edatrexate, gemcitabine, vinorelbine. When used in combination with certain chemotherapeutic agents, most notably cisplatin, carboplatin, paclitaxel, docetaxel, and edatrexate, marked response was observed compared to treatment with these agents alone, resulting in partial or complete regression in some cases. The above studies provide proof that inhibition of EGFR expression by anti-EGFR agents causes inhibition of tumor growth in animals.

Animal Model Development

Tumor cell lines (A549 and SKOV-3) are characterized to establish their growth curves in mice. These cell lines are implanted into both nude and SCID mice and primary tumor volumes are measured 3 times per week. Growth characteristics of these tumor lines using a Matrigel implantation format can also be established. The use of other cell lines that have been engineered to express high levels of EGFR can also be used in the described studies. The tumor cell line(s) and implantation method that supports the most consistent and reliable tumor growth is used in animal studies testing the lead EGFR nucleic acid(s). Nucleic acids are administered by daily subcutaneous injection or by continuous subcutaneous infusion from Alzet mini osmotic pumps beginning 3 days after tumor implantation and continuing for the duration of the study. Group sizes of at least 10 animals are employed. Efficacy is determined by statistical comparison of tumor volume of nucleic acid-treated animals to a control group of animals treated with saline alone. Because the growth of these tumors is generally slow (45-60 days), an initial endpoint is the time in days it takes to establish an easily measurable primary tumor (i.e. 50-100 mm³) in the presence or absence of nucleic acid treatment.

EGFR Protein Levels for Patient Screening and as a Potential Endpoint

Because elevated EGFR levels can be detected in several cancers, cancer patients can be pre-screened for elevated EGFR prior to admission to initial clinical trials testing an anti-EGFR nucleic acid. Initial EGFR levels can be determined (by ELISA) from tumor biopsies or resected tumor samples. During clinical trials, it may be possible to monitor circulating EGFR protein by ELISA. Evaluation of serial blood/serum samples over the course of the anti-EGFR nucleic acid treatment period could be useful in determining early indications of efficacy.

### Example 7

### Activity of Nucleic Acid Molecules used to Down-Regulate EGFR Gene Expression

Applicant has designed, synthesized and tested several nucleic acid molecules targeted against EGFR RNA in cell proliferation and RNA reduction assays described herein.

Proliferation Assay

The model proliferation assay used in the study requires a cell-plating density of 2,000-10,000 cells/well in 96-well plates and at least 2 cell doublings over a 5-day treatment period. Cells used in proliferation studies were either lung or ovarian cancer cells (A549 and SKOV-3 cells respectively). To calculate cell density for proliferation assays, the FIPS (fluoro-imaging processing system) method known in the art was used. This method allows for cell density measurements after nucleic acids are stained with CyQuant® dye, and has the advantage of accurately measuring cell densities over a very wide range 1,000-100,000 cells/well in 96-well format.

Enzymatic nucleic acid molecules and GeneBlocs (50-200 nM) were delivered in the presence of cationic lipid at 2.5-5.0 μg/mL and inhibition of proliferation was determined on day 5 post-treatment. FIGS. 6 and 7 provide examples of anti-EGFR proliferation screens in A549 cells and SKOV3 cells respectively compared to untreated controls. Additional controls used in the screens include a SAC control (RPI 21083), a transfection control (Tf Control, lipid without nucleic acid), and GeneBloc control (RPI 11698). Table IX shows a comparison of different nucleic acid molecules of the invention in proliferation screens. This data shows that nucleic acid molecules of the invention are capable of down-regulating EGFR gene expression in mammalian cells. Enzymatic nucleic acid RPI 21256 can be used to target both EGFR and HER2 gene expression.

RNA Assay

RNA was harvested 24 hours post-treatment using the Qiagen RNeasyg 96 procedure.

Real time RT-PCR (TaqMan® assay) was performed on purified RNA samples using separate primer/probe sets specific for target EGFR RNA. FIGS. 8 and 9 show results of RNA screens in A549 and SKOV3 cells respectively compared to untreated controls. Additional controls used in the screens include a SAC control (RPI 21083), a transfection control (Tf Control, lipid without nucleic acid), and GeneBloc control (RPI 11698).

### Example 8

### Activity of Nucleic Acid Molecules used to Down-Regulate EGFR and HER2 Gene Expression

Applicant has designed, synthesized and tested nucleic acid molecules that target both EGFR and HER2 RNA in cell proliferation and RNA reduction assays described herein. The use a single nucleic acid molecule that can target both EGFR and HER2 RNA in a sequence specific manner can be advantageous in inhibiting the expression of two proteins that are up-regulated in a variety of cancers. Furthermore, Brandt et al., 1999, FASEB. J., 13, 1939-1949, propose that HER2 and EGFR are dominant heterodimer partners that determine a motogenic phonotype in human breast cancer cells. The use of nucleic acid molecules that target both EGFR and HER2 RNA is advantageous since only one composition is used to inhibit both targets and can potentially provide a synergistic or additive therapeutic effect. As shown in FIGS. 6 and 7, RPI 21256, an enzymatic nucleic acid that targets both HER2 and EGFR, provides significant antiproliferative activity in both A549 and SKOV3 cells.

Indications

Particular degenerative and disease states that can be associated with EGFR expression modulation include but are not limited to cancers and cancerous conditions such as breast, lung, prostate, colorectal, brain, esophageal, stomach, bladder, pancreatic, cervical, head and neck, and ovarian cancer, melanoma, lymphoma, glioma, multidrug resistant cancers, and any other diseases or conditions that are related to or will respond to the levels of EGFR in a cell or tissue, alone or in combination with other therapies.

The present body of knowledge in EGFR research indicates the need for methods to assay EGFR activity and for compounds that can regulate EGFR expression for research, diagnostic, and therapeutic use.

The use of monoclonal antibodies (eg; mAb IMC C225, mAB ABX-EGF) treatment, EGFR-specific tyrosine kinase inhibitors (TKIs), for example OSI-774 and ZDI839, chemotherapy, and/or radiation therapy, are all non-limiting examples of a methods that can be combined with or used in conjunction with the nucleic acid molecules (e.g. ribozymes and antisense molecules) of the instant invention. Common chemotherapies that can be combined with nucleic acid molecules of the instant invention include various combinations of cytotoxic drugs to kill the cancer cells. These drugs include but are not limited to paclitaxel (Taxol), docetaxel, cisplatin, methotrexate, cyclophosphamide, doxorubin, fluorouracil carboplatin, edatrexate, gemcitabine, vinorelbine etc. Those skilled in the art will recognize that other drug compounds and therapies can be similarly be readily combined with the nucleic acid molecules of the instant invention (e.g. ribozymes and antisense molecules) are hence within the scope of the instant invention.

Diagnostic Uses

The nucleic acid molecules of this invention (e.g., enzymatic nucleic acid molecules) can be used as diagnostic tools to examine genetic drift and mutations within diseased cells or to detect the presence of EGFR RNA in a cell. The close relationship between enzymatic nucleic acid molecule activity and the structure of the target RNA allows the detection of mutations in any region of the molecule which alters the base-pairing and three-dimensional structure of the target RNA. By using multiple enzymatic nucleic acid molecules described in this invention, one can map nucleotide changes which are important to RNA structure and function in vitro, as well as in cells and tissues. Cleavage of target RNAs with enzymatic nucleic acid molecules can be used to inhibit gene expression and define the role (essentially) of specified gene products in the progression of disease. In this manner, other genetic targets can be defined as important mediators of the disease. These experiments can lead to better treatment of the disease progression by affording the possibility of combinational therapies (e.g., multiple enzymatic nucleic acid molecules targeted to different genes, enzymatic nucleic acid molecules coupled with known small molecule inhibitors, or intermittent treatment with combinations of enzymatic nucleic acid molecules and/or other chemical or biological molecules). Other in vitro uses of enzymatic nucleic acid molecules of this invention are well known in the art, and include detection of the presence of mRNAs associated with EGFR-related condition. Such RNA is detected by determining the presence of a cleavage product after treatment with an enzymatic nucleic acid molecule using standard methodology.

In a specific example, enzymatic nucleic acid molecules which cleave only wild-type or mutant forms of the target RNA are used for the assay. The first enzymatic nucleic acid molecule is used to identify wild-type RNA present in the sample and the second enzymatic nucleic acid molecule is used to identify mutant RNA in the sample. As reaction controls, synthetic substrates of both wild-type and mutant RNA are cleaved by both enzymatic nucleic acid molecules to demonstrate the relative enzymatic nucleic acid molecule efficiencies in the reactions and the absence of cleavage of the “non-targeted” RNA species. The cleavage products from the synthetic substrates also serve to generate size markers for the analysis of wild-type and mutant RNAs in the sample population. Thus each analysis requires two enzymatic nucleic acid molecules, two substrates and one unknown sample which is combined into six reactions. The presence of cleavage products is determined using an RNAse protection assay so that full-length and cleavage fragments of each RNA can be analyzed in one lane of a polyacrylamide gel. It is not absolutely required to quantify the results to gain insight into the expression of mutant RNAs and putative risk of the desired phenotypic changes in target cells. The expression of mRNA whose protein product is implicated in the development of the phenotype (i.e., EGFR) is adequate to establish risk. If probes of comparable specific activity are used for both transcripts, then a qualitative comparison of RNA levels will be adequate and will decrease the cost of the initial diagnosis. Higher mutant form to wild-type ratios are correlated with higher risk whether RNA levels are compared qualitatively or quantitatively. The use of enzymatic nucleic acid molecules in diagnostic applications contemplated by the instant invention is more fully described in George et al., U.S. Pat. Nos. 5,834,186 and 5,741,679, Shih et al., U.S. Pat. No. 5,589,332, Nathan et al., U.S. Pat. No 5,871,914, Nathan and Ellington, International PCT publication No. WO 00/24931, Breaker et al., International PCT Publication Nos. WO 00/26226 and 98/27104, and Sullenger et al., International PCT publication No. WO 99/29842.

Additional Uses

Potential uses of sequence-specific enzymatic nucleic acid molecules of the instant invention can have many of the same applications for the study of RNA that DNA restriction endonucleases have for the study of DNA (Nathans et al., 1975 Ann. Rev. Biochem. 44:273). For example, the pattern of restriction fragments can be used to establish sequence relationships between two related RNAs, and large RNAs can be specifically cleaved to fragments of a size more useful for study. The ability to engineer sequence specificity of the enzymatic nucleic acid molecule is ideal for cleavage of RNAs of unknown sequence. Applicant has described the use of nucleic acid molecules to down-regulate gene expression of target genes in bacterial, microbial, fungal, viral, and eukaryotic systems including plant, or mammalian cells.

All patents and publications mentioned in the specification are indicative of the levels of skill of those skilled in the art to which the invention pertains. All references cited in this disclosure are incorporated by reference to the same extent as if each reference had been incorporated by reference in its entirety individually.

One skilled in the art would readily appreciate that the present invention is well adapted to carry out the objects and obtain the ends and advantages mentioned, as well as those inherent therein. The methods and compositions described herein as presently representative of preferred embodiments are exemplary and are not intended as limitations on the scope of the invention. Changes therein and other uses will occur to those skilled in the art, which are encompassed within the spirit of the invention, are defined by the scope of the claims.

It will be readily apparent to one skilled in the art that varying substitutions and modifications can be made to the invention disclosed herein without departing from the scope and spirit of the invention. Thus, such additional embodiments are within the scope of the present invention and the following claims.

The invention illustratively described herein suitably can be practiced in the absence of any element or elements, limitation or limitations which is not specifically disclosed herein. Thus, for example, in each instance herein any of the terms “comprising”, “consisting essentially of” and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments, optional features, modification and variation of the concepts herein disclosed may be resorted to by those skilled in the art, and that such modifications and variations are considered to be within the scope of this invention as defined by the description and the appended claims.

In addition, where features or aspects of the invention are described in terms of Markush groups or other grouping of alternatives, those skilled in the art will recognize that the invention is also thereby described in terms of any individual member or subgroup of members of the Markush group or other group.

Other embodiments are within the claims that follow.

TABLE I
Characteristics of naturally occurring ribozymes

Group I Introns
Size: ˜150 to >1000 nucleotides.
Requires a U in the target sequence immediately 5′ of the cleavage site.
Binds 4-6 nucleotides at the 5′-side of the cleavage site.
Reaction mechanism: attack by the 3′-OH of guanosine to generate
cleavage products with 3′-OH and 5′-guanosine.
Additional protein cofactors required in some cases to help folding and
maintenance of the active structure.
Over 300 known members of this class. Found as an intervening sequence
in Tetrahymena thermophila rRNA, fungal mitochondria, chloroplasts,
phage T4, blue-green algae, and others.
Major structural features largely established through phylogenetic
comparisons, mutagenesis, and biochemical studies [ⁱ,ⁱⁱ].
Complete kinetic framework established for one ribozyme [ⁱⁱⁱ,^{iv},^{v},^{vi}].
Studies of ribozyme folding and substrate docking underway [^{vii},^{viii},^{ix}].
Chemical modification investigation of important residues well established
[^{x},^{xi}].
The small (4-6 nt) binding site may make this ribozyme too non-specific
for targeted RNA cleavage, however, the Tetrahymena group I intron has
been used to repair a “defective” β-galactosidase message by the ligation
of new β-galactosidase sequences onto the defective message [^{xii}].
RNAse P RNA (M1 RNA)
Size: ˜290 to 400 nucleotides.
RNA portion of a ubiquitous ribonucleoprotein enzyme.
Cleaves tRNA precursors to form mature tRNA [^{xiii}].
Reaction mechanism: possible attack by M²⁺-OH to generate cleavage
products with 3′-OH and 5′-phosphate.
RNAse P is found throughout the prokaryotes and eukaryotes. The RNA
subunit has been sequenced from bacteria, yeast, rodents, and primates.
Recruitment of endogenous RNAse P for therapeutic applications is
possible through hybridization of an External Guide Sequence (EGS) to
the target RNA [^{xiv},^{xv}]
Important phosphate and 2′ OH contacts recently identified [^{xvi},^{xvii}]
Group II Introns
Size: >1000 nucleotides.
Trans cleavage of target RNAs recently demonstrated [^{xviii},^{xix}].
Sequence requirements not fully determined.
Reaction mechanism: 2′-OH of an internal adenosine generates cleavage
products with 3′-OH and a “lariat” RNA containing a 3′-5′ and a 2′-5′
branch point.
Only natural ribozyme with demonstrated participation in DNA cleavage
[^{xx},^{xxi}] in addition to RNA cleavage and ligation.
Major structural features largely established through phylogenetic
comparisons [^{xxii}]
Important 2′ OH contacts beginning to be identified [^{xxiii}]
Kinetic framework under development [^{xxiv}]
Neurospora VS RNA
Size: ˜144 nucleotides.
Trans cleavage of hairpin target RNAs recently demonstrated [^{xxv}].
Sequence requirements not fully determined.
Reaction mechanism: attack by 2′-OH 5′ to the scissile bond to generate
cleavage products with 2′,3′-cyclic phosphate and 5′-OH ends.
Binding sites and structural requirements not fully determined.
Only 1 known member of this class. Found in Neurospora VS RNA.
Hammerhead Ribozyme
(see text for references)
Size: ˜13 to 40 nucleotides.
Requires the target sequence UH immediately 5′ of the cleavage site.
Binds a variable number nucleotides on both sides of the cleavage site.
Reaction mechanism: attack by 2′-OH 5′ to the scissile bond to generate
cleavage products with 2′,3′-cyclic phosphate and 5′-OH ends.
14 known members of this class. Found in a number of plant pathogens
(virusoids) that use RNA as the infectious agent.
Essential structural features largely defined, including 2 crystal structures
[^{xxvi},^{xxvii}]
Minimal ligation activity demonstrated (for engineering through in vitro
selection) [^{xxviii}]
Complete kinetic framework established for two or more ribozymes [^{xxix}].
Chemical modification investigation of important residues well established
[^{xxx}].
Hairpin Ribozyme
Size: ˜50 nucleotides.
Requires the target sequence GUC immediately 3′ of the cleavage site.
Binds 4-6 nucleotides at the 5′-side of the cleavage site and a variable
number to the 3′-side of the cleavage site.
Reaction mechanism: attack by 2′-OH 5′ to the scissile bond to generate
cleavage products with 2′,3′-cyclic phosphate and 5′-OH ends.
3 known members of this class. Found in three plant pathogen (satellite
RNAs of the tobacco ringspot virus, arabis mosaic virus and chicory
yellow mottle virus) which uses RNA as the infectious agent.
Essential structural features largely defined [^{xxxi},^{xxxii},^{xxxiii},^{xxxiv}]
Ligation activity (in addition to cleavage activity) makes ribozyme
amenable to engineering through in vitro selection [^{xxxv}]
Complete kinetic framework established for one ribozyme [^{xxxvi}].
Chemical modification investigation of important residues begun
[^{xxxvii},^{xxxviii}].
Hepatitis Delta Virus (HDV) Ribozyme
Size: ˜60 nucleotides.
Trans cleavage of target RNAs demonstrated [^{xxxix}].
Binding sites and structural requirements not fully determined, although no
sequences 5′ of cleavage site are required. Folded ribozyme contains a
pseudoknot structure [^{xl}]
Reaction mechanism: attack by 2′-OH 5′ to the scissile bond to generate
cleavage products with 2′,3′-cyclic phosphate and 5′-OH ends.
Only 2 known members of this class. Found in human HDV.
Circular form of HDV is active and shows increased nuclease stability [^{xli}]
ⁱ Michel, Francois; Westhof, Eric. Slippery substrates. Nat. Struct. Biol. (1994), 1(1), 5-7.
ⁱⁱ Lisacek, Frederique; Diaz, Yolande; Michel, Francois. Automatic identification of group I intron cores in genomic DNA sequences. J. Mol. Biol. (1994), 235(4), 1206-17.
ⁱⁱⁱ Herschlag, Daniel; Cech, Thomas R.. Catalysis of RNA cleavage by the Tetrahymena thermophila ribozyme. 1. Kinetic description of the reaction of an RNA substrate complementary to the active site. Biochemistry (1990), 29(44), 10159-71.
^{iv} Herschlag, Daniel; Cech, Thomas R.. Catalysis of RNA cleavage by the Tetrahymena thermophila ribozyme. 2. Kinetic description of the reaction of an RNA substrate that forms a mismatch at the active site. Biochemistry (1990), 29(44), 10172-80.
^{v} Knitt, Deborah S.; Herschlag, Daniel. pH Dependencies of the Tetrahymena Ribozyme Reveal an Unconventional Origin of an Apparent pKa. Biochemistry (1996), 35(5), 1560-70.
^{vi} Bevilacqua, Philip C.; Sugimoto, Naoki; Turner, Douglas H.. A mechanistic framework for the second step of splicing catalyzed by the Tetrahymena ribozyme. Biochemistry (1996), 35(2), 648-58.
^{vii} Li, Yi; Bevilacqua, Philip C.; Mathews, David; Turner, Douglas H.. Thermodynamic and activation parameters for binding of a pyrene-labeled substrate by the Tetrahymena ribozyme: docking is not diffusion-controlled and is driven by a favorable entropy change. Biochemistry (1995), 34(44), 14394-9.
^{viii} Banerjee, Aloke Raj; Turner, Douglas H.. The time dependence of chemical modification reveals slow steps in the folding of a group I ribozyme. Biochemistry (1995), 34(19), 6504-12.
^{ix} Zarrinkar, Patrick P.; Williamson, James R.. The P9.1-P9.2 peripheral extension helps guide folding of the Tetrahymena ribozyme. Nucleic Acids Res. (1996), 24(5), 854-8.
^{x} Strobel, Scott A.; Cech, Thomas R.. Minor groove recognition of the conserved G.cntdot.U pair at the Tetrahymena ribozyme reaction site. Science (Washington, D.C.) (1995), 267(5198), 675-9.
^{xi} Strobel, Scott A.; Cech, Thomas R.. Exocyclic Amine of the Conserved G.cntdot.U Pair at the Cleavage Site of the Tetrahymena Ribozyme Contributes to 5′-Splice Site Selection and Transition State Stabilization. Biochemistry (1996), 35(4), 1201-11.
^{xii} Sullenger, Bruce A.; Cech, Thomas R.. Ribozyme-mediated repair of defective mRNA by targeted trans-splicing. Nature (London) (1994), 371(6498), 619-22.
^{xiii} Robertson, H.D.; Altman, S.; Smith,J.D. J. Biol. Chem., 247, 5243-5251 (1972).
^{xiv} Forster, Anthony C.; Altman, Sidney. External guide sequences for an RNA enzyme. Science (Washington, D.C., 1883-) (1990), 249(4970), 783-6.
^{xv} Yuan, Y.; Hwang, E.S.; Altman, S. Targeted cleavage of mRNA by human RNase P. Proc. Natl. Acad. Sci. USA (1992) 89, 8006-10.
^{xvi} Harris, Michael E.; Pace, Norman R.. Identification of phosphates involved in catalysis by the ribozyme RNase P RNA. RNA (1995), 1(2), 210-18.
^{xvii} Pan, Tao; Loria, Andrew; Zhong, Kun. Probing of tertiary interactions in RNA: 2′-hydroxyl-base contacts between the RNase P RNA and pre-tRNA. Proc. Natl. Acad. Sci. U.S.A. (1995), 92(26), 12510-14.
^{xviii} Pyle, Anna Marie; Green, Justin B.. Building a Kinetic Framework for Group II Intron Ribozyme Activity: Quantitation of Interdomain Binding and Reaction Rate. Biochemistry (1994), 33(9), 2716-25.
^{xix} Michels, William J. Jr.; Pyle, Anna Marie. Conversion of a Group II Intron into a New Multiple-Turnover Ribozyme that Selectively Cleaves Oligonucleotides: Elucidation of Reaction Mechanism and Structure/Function Relationships. Biochemistry (1995), 34(9), 2965-77.
^{xx} Zimmerly, Steven; Guo, Huatao; Eskes, Robert; Yang, Jian; Perlman, Philip S.; Lambowitz, Alan M.. A group II intron RNA is a catalytic component of a DNA endonuclease involved in intron mobility. Cell (Cambridge, Mass.) (1995), 83(4), 529-38.
^{xxi} Griffin, Edmund A., Jr.; Qin, Zhifeng; Michels, Williams J., Jr.; Pyle, Anna Marie. Group II intron ribozymes that cleave DNA and RNA linkages with similar efficiency, and lack contacts with substrate 2′-hydroxyl groups. Chem. Biol. (1995), 2(11), 761-70.
^{xxii} Michel, Francois; Ferat, Jean Luc. Structure and activities of group II introns. Annu. Rev. Biochem. (1995), 64, 435-61.
^{xxiii} Abramovitz, Dana L.; Friedman, Richard A.; Pyle, Anna Marie. Catalytic role of 2′-hydroxyl groups within a group II intron active site. Science (Washington, D.C.) (1996), 271(5254), 1410-13.
^{xxiv} Daniels, Danette L.; Michels, William J., Jr.; Pyle, Anna Marie. Two competing pathways for self-splicing by group II introns: a quantitative analysis of in vitro reaction rates and products. J. Mol. Biol. (1996), 256(1), 31-49.
^{xxv} Guo, Hans C.T.; Collins, Richard A.. Efficient trans-cleavage of a stem-loop RNA substrate by a ribozyme derived from Neurospora VS RNA. EMBO J. (1995), 14(2), 368-76.
^{xxvi} Scott, W.G., Finch, J.T., Aaron,K. The crystal structure of an all RNA hammerhead ribozyme:Aproposed mechanism for RNA catalytic cleavage. Cell, (1995), 81, 991-1002.
^{xxvii} McKay, Structure and function of the hammerhead ribozyme: an unfinished story. RNA, (1996), 2, 395-403.
^{xxviii} Long, D., Uhlenbeck, 0., Hertel, K. Ligation with hammerhead ribozymes. U.S. Pat. No. 5,633,133.
^{xxix} Hertel, K.J., Herschlag, D., Uhlenbeck, O. A kinetic and thermodynamic framework for the hammerhead ribozyme reaction. Biochemistry, (1994) 33, 3374-3385.Beigelman, L., et al., Chemical modifications of hammerhead ribozymes. J. Biol. Chem., (1995) 270, 25702-25708.
^{xxx} Beigelman, L., et al., Chemical modifications of hammerhead ribozymes. J. Biol. Chem., (1995) 270, 25702-25708.
^{xxxi} Hampel, Arnold; Tritz, Richard; Hicks, Margaret; Cruz, Phillip. ‘Hairpin’ catalytic RNA model: evidence for helixes and sequence requirement for substrate RNA. Nucleic Acids Res. (1990), 18(2), 299-304.
^{xxxii} Chowrira, Bharat M.; Berzal-Herranz, Alfredo; Burke, John M.. Novel guanosine requirement for catalysis by the hairpin ribozyme. Nature (London) (1991), 354(6351), 320-2.
^{xxxiii} Berzal-Herranz, Alfredo; Joseph, Simpson; Chowrira, Bharat M.; Butcher, Samuel E.; Burke, John M.. Essential nucleotide sequences and secondary structure elements of the hairpin ribozyme. EMBO J. (1993), 12(6), 2567-73.
^{xxxiv} Joseph, Simpson; Berzal-Herranz, Alfredo; Chowrira, Bharat M.; Butcher, Samuel E.. Substrate selection rules for the hairpin ribozyme determined by in vitro selection, mutation, and analysis of mismatched substrates. Genes Dev. (1993), 7(1), 130-8.
^{xxxv} Berzal-Herranz, Alfredo; Joseph, Simpson; Burke, John M.. In vitro selection of active hairpin ribozymes by sequential RNA-catalyzed cleavage and ligation reactions. Genes Dev. (1992), 6(1), 129-34.
^{xxxvi} Hegg, Lisa A.; Fedor, Martha J.. Kinetics and Thermodynamics of Intermolecular Catalysis by Hairpin Ribozymes. Biochemistry (1995), 34(48), 15813-28.
^{xxxvii} Grasby, Jane A.; Mersmann, Karin; Singh, Mohinder; Gait, Michael J.. Purine Functional Groups in Essential Residues of the Hairpin Ribozyme Required for Catalytic Cleavage of RNA. Biochemistry (1995), 34(12), 4068-76.
^{xxxviii} Schmidt, Sabine; Beigelman, Leonid; Karpeisky, Alexander; Usman, Nassim; Sorensen, Ulrik S.; Gait, Michael J.. Base and sugar requirements for RNA cleavage of essential nucleoside residues in internal loop B of the hairpin ribozyme: implications for secondary structure. Nucleic Acids Res. (1996), 24(4), 573-81.
^{xxxix} Perrotta, Anne T.; Been, Michael D.. Cleavage of oligoribonucleotides by a ribozyme derived from the hepatitis delta. virus RNA sequence. Biochemistry (1992), 31(1), 16-21.
^{xl} Perrotta, Anne T.; Been, Michael D.. A pseudoknot-like structure required for efficient self-cleavage of hepatitis delta virus RNA. Nature (London) (1991), 350(6317), 434-6.
^{xli} Puttaraju, M.; Perrotta, Anne T.; Been, Michael D.. A circular trans-acting hepatitis delta virus ribozyme. Nucleic Acids Res. (1993), 21(18), 4253-8.

TABLE II

| | | | | | |
|---|---|---|---|---|---|
| | | | | Wait Time* 2'- | Wait Time* |
| Reagent | Equivalents | Amount | Wait Time* DNA | O-methyl | RNA |

A. 2.5 μmol Synthesis Cycle ABI 394 Instrument

| | | | | | | |
|---|---|---|---|---|---|---|
| Phosphoramidites | 6.5 | 163 | μL | 45 sec | 2.5 min | 7.5 min |
| S-Ethyl Tetrazole | 23.8 | 238 | μL | 45 sec | 2.5 min | 7.5 min |
| Acetic Anhydride | 100 | 233 | μL | 5 sec | 5 sec | 5 sec |
| N-Methyl Imidazole | 186 | 233 | μL | 5 sec | 5 sec | 5 sec |
| TCA | 176 | 2.3 | mL | 21 sec | 21 sec | 21 sec |
| Iodine | 11.2 | 1.7 | mL | 45 sec | 45 sec | 45 sec |
| Beaucage | 12.9 | 645 | μL | 100 sec | 300 sec | 300 sec |
| Acetonitrile | NA | 6.67 | mL | NA | NA | NA |

B. 0.2 μmol Synthesis Cycle ABI 394 Instrument

| | | | | | | |
|---|---|---|---|---|---|---|
| Phosphoramidites | 15 | 31 | μL | 45 sec | 233 sec | 465 sec |
| S-Ethyl Tetrazole | 38.7 | 31 | μL | 45 sec | 233 min | 465 sec |
| Acetic Anhydride | 655 | 124 | μL | 5 sec | 5 sec | 5 sec |
| N-Methyl Imidazole | 1245 | 124 | μL | 5 sec | 5 sec | 5 sec |
| TCA | 700 | 732 | μL | 10 sec | 10 sec | 10 sec |
| Iodine | 20.6 | 244 | μL | 15 sec | 15 sec | 15 sec |
| Beaucage | 7.7 | 232 | μL | 100 sec | 300 sec | 300 sec |
| Acetonitrile | NA | 2.64 | mL | NA | NA | NA |

| | | | | | |
|---|---|---|---|---|---|
| | Equivalents | Amount | | | |
| | DNA/2'-O- | DNA/2'-O-methyl/ | Wait Time* | Wait Time* | Wait Time* |
| Reagent | methyl/Ribo | Ribo | DNA | 2'-O-methyl | Ribo |

C. 0.2 μmol Synthesis Cycle 96 well Instrument

| | | | | | |
|---|---|---|---|---|---|
| Phosphoramidites | 22/33/66 | 40/60/120 μL | 60 sec | 180 sec | 360 sec |
| S-Ethyl Tetrazole | 70/105/210 | 40/60/120 μL | 60 sec | 180 min | 360 sec |
| Acetic Anhydride | 265/265/265 | 50/50/50 μL | 10 sec | 10 sec | 10 sec |
| N-Methyl Imidazole | 502/502/502 | 50/50/50 μL | 10 sec | 10 sec | 10 sec |
| TCA | 238/475/475 | 250/500/500 μL | 15 sec | 15 sec | 15 sec |
| Iodine | 6.8/6.8/6.8 | 80/80/80 μL | 30 sec | 30 sec | 30 sec |
| Beaucage | 34/51/51 | 80/120/120 | 100 sec | 200 sec | 200 sec |
| Acetonitrile | NA | 1150/1150/1150 μL | NA | NA | NA |

*Wait time does not include contact time during delivery.

TABLE III

Human EGFR Receptor Hammerhead Rihozyme and Substrate
Sequence

| | | | | |
|---|---|---|---|---|
| Pos | Substrate | Seq ID | Ribozyme | Seq ID |

| | | | | |
|---|---|---|---|---|
| 19 | GCCGGAGU C CCGAGCUA | 1 | UAGCUCGG CUGAUGAGGCCGUUAGGCCGAA ACUCCGGC | 3778 |
| 27 | CCCGAGCU A GCCCCGGC | 2 | GCCGGGGC CUGAUGAGGCCGUUAGGCCGAA AGCUCGGG | 3779 |
| 70 | GGCCACCU C GUCGGCGU | 3 | ACGCCGAC CUGAUGAGGCCGUUAGGCCGAA AGGUGGCC | 3780 |
| 73 | CACCUCGU C GGCGUCCG | 4 | CGGACGCC CUGAUGAGGCCGUUAGGCCGAA ACGAGGUG | 3781 |
| 79 | GUCGGCGU C CGCCCGAG | 5 | CUCGGGCG CUGAUGAGGCCGUUAGGCCGAA ACGCCGAC | 3782 |
| 89 | GCCCGAGU C CCCGCCUC | 6 | GAGGCGGG CUGAUGAGGCCGUUAGGCCGAA ACUCGGGC | 3783 |
| 97 | CCCCGCCU C GCCGCCAA | 7 | UUGGCGGC CUGAUGAGGCCGUUAGGCCGAA AGGCGGGG | 3784 |
| 137 | CCCUGACU C CGUCCAGU | 8 | ACUGGACG CUGAUGAGGCCGUUAGGCCGAA AGUCAGGG | 3785 |
| 141 | GACUCCGU C CAGUAUUG | 9 | CAAUACUG CUGAUGAGGCCGUUAGGCCGAA ACGGAGUC | 3786 |
| 146 | CGUCCAGU A UUGAUCGG | 10 | CCGAUCAA CUGAUGAGGCCGUUAGGCCGAA ACUGGACG | 3787 |
| 148 | UCCAGUAU U GAUCGGGA | 11 | UCCCGAUC CUGAUGAGGCCGUUAGGCCGAA AUACUGGA | 3788 |
| 152 | GUAUUGAU C GGGAGAGC | 12 | GCUCUCCC CUGAUGAGGCCGUUAGGCCGAA AUCAAUAC | 3789 |
| 172 | AGCGAGCU C UUCGGGGA | 13 | UCCCCGAA CUGAUGAGGCCGUUAGGCCGAA AGCUCGCU | 3790 |
| 174 | CGAGCUCU U CGGGGAGC | 14 | GCUCCCCG CUGAUGAGGCCGUUAGGCCGAA AGAGCUCG | 3791 |
| 175 | GAGCUCUU C GGGGAGCA | 15 | UGCUCCCC CUGAUGAGGCCGUUAGGCCGAA AAGAGCUC | 3792 |
| 197 | GCGACCCU C CGGGACGG | 16 | CCGUCCCG CUGAUGAGGCCGUUAGGCCGAA AGGGUCGC | 3793 |
| 219 | GCAGCGCU C CUGGCGCU | 17 | AGCGCCAG CUGAUGAGGCCGUUAGGCCGAA AGCGCUGC | 3794 |
| 240 | GCUGCGCU C UGCCCGGC | 18 | GCCGGGCA CUGAUGAGGCCGUUAGGCCGAA AGCGCAGC | 3795 |
| 253 | CGGCGAGU C GGGCUCUG | 19 | CAGAGCCC CUGAUGAGGCCGUUAGGCCGAA ACUCGCCG | 3796 |
| 259 | GUCGGGCU C UGGAGGAA | 20 | UUCCUCCA CUGAUGAGGCCGUUAGGCCGAA AGCCCGAC | 3797 |
| 276 | AAGAAAGU U UGCCAAGG | 21 | CCUUGGCA CUGAUGAGGCCGUUAGGCCGAA ACUUUCUU | 3798 |
| 277 | AGAAAGUU U GCCAAGGC | 22 | GCCUUGGC CUGAUGAGGCCGUUAGGCCGAA AACUUUCU | 3799 |
| 292 | GCACGAGU A ACAAGCUC | 23 | GAGCUUGU CUGAUGAGGCCGUUAGGCCGAA ACUCGUGC | 3800 |
| 300 | AACAAGCU C ACGCAGUU | 24 | AACUGCGU CUGAUGAGGCCGUUAGGCCGAA AGCUUGUU | 3801 |
| 308 | CACGCAGU U GGGCACUU | 25 | AAGUGCCC CUGAUGAGGCCGUUAGGCCGAA ACUGCGUG | 3802 |
| 316 | UGGGCACU U UUGAAGAU | 26 | AUCUUCAA CUGAUGAGGCCGUUAGGCCGAA AGUGCCCA | 3803 |
| 317 | GGGCACUU U UGAAGAUC | 27 | GAUCUUCA CUGAUGAGGCCGUUAGGCCGAA AAGUGCCC | 3804 |
| 318 | GGCACUUU U GAAGAUCA | 28 | UGAUCUUC CUGAUGAGGCCGUUAGGCCGAA AAAGUGCC | 3805 |
| 325 | UUGAAGAU C AUUUUCUC | 29 | GAGAAAAU CUGAUGAGGCCGUUAGGCCGAA AUCUUCAA | 3806 |
| 328 | AAGAUCAU U UUCUCAGC | 30 | GCUGAGAA CUGAUGAGGCCGUUAGGCCGAA AUGAUCUU | 3807 |
| 329 | AGAUCAUU U UCUCAGCC | 31 | GGCUGAGA CUGAUGAGGCCGUUAGGCCGAA AAUGAUCU | 3808 |
| 330 | GAUCAUUU U CUCAGCCU | 32 | AGGCUGAG CUGAUGAGGCCGUUAGGCCGAA AAAUGAUC | 3809 |
| 331 | AUCAUUUU C UCAGCCUC | 33 | GAGGCUGA CUGAUGAGGCCGUUAGGCCGAA AAAAUGAU | 3810 |
| 333 | CAUUUUCU C AGCCUCCA | 34 | UGGAGGCU CUGAUGAGGCCGUUAGGCCGAA AGAAAAUG | 3811 |
| 339 | CUCAGCCU C CAGAGGAU | 35 | AUCCUCUG CUGAUGAGGCCGUUAGGCCGAA AGGCUGAG | 3812 |
| 350 | GAGGAUGU U CAAUAACU | 36 | AGUUAUUG CUGAUGAGGCCGUUAGGCCGAA ACAUCCUC | 3813 |
| 351 | AGGAUGUU C AAUAACUG | 37 | CAGUUAUU CUGAUGAGGCCGUUAGGCCGAA AACAUCCU | 3814 |
| 355 | UGUUCAAU A ACUGUGAG | 38 | CUCACAGU CUGAUGAGGCCGUUAGGCCGAA AUUGAACA | 3815 |
| 369 | GAGGUGGU C CUUGGGAA | 39 | UUCCCAAG CUGAUGAGGCCGUUAGGCCGAA ACCACCUC | 3816 |
| 372 | GUGGUCCU U GGGAAUUU | 40 | AAAUUCCC CUGAUGAGGCCGUUAGGCCGAA AGGACCAC | 3817 |
| 379 | UUGGGAAU U UGGAAAUU | 41 | AAUUUCCA CUGAUGAGGCCGUUAGGCCGAA AUUCCCAA | 3818 |
| 380 | UGGGAAUU U GGAAAUUA | 42 | UAAUUUCC CUGAUGAGGCCGUUAGGCCGAA AAUUCCCA | 3819 |
| 387 | UUGGAAAU U ACCUAUGU | 43 | ACAUAGGU CUGAUGAGGCCGUUAGGCCGAA AUUUCCAA | 3820 |
| 388 | UGGAAAUU A CCUAUGUG | 44 | CACAUAGG CUGAUGAGGCCGUUAGGCCGAA AAUUUCCA | 3821 |
| 392 | AAUUACCU A UGUGCAGA | 45 | UCUGCACA CUGAUGAGGCCGUUAGGCCGAA AGGUAAUU | 3822 |
| 406 | AGAGGAAU U AUGAUCUU | 46 | AAGAUCAU CUGAUGAGGCCGUUAGGCCGAA AUUCCUCU | 3823 |
| 407 | GAGGAAUU A UGAUCUUU | 47 | AAAGAUCA CUGAUGAGGCCGUUAGGCCGAA AAUUCCUC | 3824 |
| 412 | AUUAUGAU C UUUCCUUC | 48 | GAAGGAAA CUGAUGAGGCCGUUAGGCCGAA AUCAUAAU | 3825 |
| 414 | UAUGAUCU U UCCUUCUU | 49 | AAGAAGGA CUGAUGAGGCCGUUAGGCCGAA AGAUCAUA | 3826 |
| 415 | AUGAUCUU U CCUUCUUA | 50 | UAAGAAGG CUGAUGAGGCCGUUAGGCCGAA AAGAUCAU | 3827 |
| 416 | UGAUCUUU C CUUCUUAA | 51 | UUAAGAAG CUGAUGAGGCCGUUAGGCCGAA AAAGAUCA | 3828 |
| 419 | UCUUUCCU U CUUAAAGA | 52 | UCUUUAAG CUGAUGAGGCCGUUAGGCCGAA AGGAAAGA | 3829 |
| 420 | CUUUCCUU C UUAAAGAC | 53 | GUCUUUAA CUGAUGAGGCCGUUAGGCCGAA AAGGAAAG | 3830 |
| 422 | UUCCUUCU U AAAGACCA | 54 | UGGUCUUU CUGAUGAGGCCGUUAGGCCGAA AGAAGGAA | 3831 |
| 423 | UCCUUCUU A AAGACCAU | 55 | AUGGUCUU CUGAUGAGGCCGUUAGGCCGAA AAGAAGGA | 3832 |
| 432 | AAGACCAU C CAGGAGGU | 56 | ACCUCCUG CUGAUGAGGCCGUUAGGCCGAA AUGGUCUU | 3833 |
| 448 | UGGCUGGU U AUGUCCUC | 57 | GAGGACAU CUGAUGAGGCCGUUAGGCCGAA ACCAGCCA | 3834 |
| 449 | GGCUGGUU A UGUCCUCA | 58 | UGAGGACA CUGAUGAGGCCGUUAGGCCGAA AACCAGCC | 3835 |
| 453 | GGUUAUGU C CUCAUUGC | 59 | GCAAUGAG CUGAUGAGGCCGUUAGGCCGAA ACAUAACC | 3836 |
| 456 | UAUGUCCU C AUUGCCCU | 60 | AGGGCAAU CUGAUGAGGCCGUUAGGCCGAA AGGACAUA | 3837 |
| 459 | GUCCUCAU U GCCCUCAA | 61 | UUGAGGGC CUGAUGAGGCCGUUAGGCCGAA AUGAGGAC | 3838 |
| 465 | AUUGCCCU C AACACAGU | 62 | ACUGUGUU CUGAUGAGGCCGUUAGGCCGAA AGGGCAAU | 3839 |
| 483 | GAGCGAAU U CCUUUGGA | 63 | UCCAAAGG CUGAUGAGGCCGUUAGGCCGAA AUUCGCUC | 3840 |
| 484 | AGCGAAUU C CUUUGGAA | 64 | UUCCAAAG CUGAUGAGGCCGUUAGGCCGAA AAUUCGCU | 3841 |
| 487 | GAAUUCCU U UGGAAAAC | 65 | GUUUUCCA CUGAUGAGGCCGUUAGGCCGAA AGGAAUUC | 3842 |
| 488 | AAUUCCUU U GGAAAACC | 66 | GGUUUUCC CUGAUGAGGCCGUUAGGCCGAA AAGGAAUU | 3843 |
| 504 | CUGCAGAU C AUCAGAGG | 67 | CCUCUGAU CUGAUGAGGCCGUUAGGCCGAA AUCUGCAG | 3844 |
| 507 | CAGAUCAU C AGAGGAAA | 68 | UUUCCUCU CUGAUGAGGCCGUUAGGCCGAA AUGAUCUG | 3845 |
| 517 | GAGGAAAU A UGUACUAC | 69 | GUAGUACA CUGAUGAGGCCGUUAGGCCGAA AUUUCCUC | 3846 |
| 521 | AAAUAUGU A CUACGAAA | 70 | UUUCGUAG CUGAUGAGGCCGUUAGGCCGAA ACAUAUUU | 3847 |
| 524 | UAUGUACU A CGAAAAUU | 71 | AAUUUUCG CUGAUGAGGCCGUUAGGCCGAA AGUACAUA | 3848 |
| 532 | ACGAAAAU U CCUAUGCC | 72 | GGCAUAGG CUGAUGAGGCCGUUAGGCCGAA AUUUUCGU | 3849 |
| 533 | CGAAAAUU C CUAUGCCU | 73 | AGGCAUAG CUGAUGAGGCCGUUAGGCCGAA AAUUUUCG | 3850 |
| 536 | AAAUUCCU A UGCCUUAG | 74 | CUAAGGCA CUGAUGAGGCCGUUAGGCCGAA AGGAAUUU | 3851 |
| 542 | CUAUGCCU U AGCAGUCU | 75 | AGACUGCU CUGAUGAGGCCGUUAGGCCGAA AGGCAUAG | 3852 |
| 543 | UAUGCCUU A GCAGUCUU | 76 | AAGACUGC CUGAUGAGGCCGUUAGGCCGAA AAGGCAUA | 3853 |
| 549 | UUAGCAGU C UUAUCUAA | 77 | UUAGAUAA CUGAUGAGGCCGUUAGGCCGAA ACUGCUAA | 3854 |
| 551 | AGCAGUCU U AUCUAACU | 78 | AGUUAGAU CUGAUGAGGCCGUUAGGCCGAA AGACUGCU | 3855 |
| 552 | GCAGUCUU A UCUAACUA | 79 | UAGUUAGA CUGAUGAGGCCGUUAGGCCGAA AAGACUGC | 3856 |
| 554 | AGUCUUAU C UAACUAUG | 80 | CAUAGUUA CUGAUGAGGCCGUUAGGCCGAA AUAAGACU | 3857 |
| 556 | UCUUAUCU A ACUAUGAU | 81 | AUCAUAGU CUGAUGAGGCCGUUAGGCCGAA AGAUAAGA | 3858 |
| 560 | AUCUAACU A UGAUGCAA | 82 | UUGCAUCA CUGAUGAGGCCGUUAGGCCGAA AGUUAGAU | 3859 |
| 571 | AUGCAAAU A AAACCGGA | 83 | UCCGGUUU CUGAUGAGGCCGUUAGGCCGAA AUUUGCAU | 3860 |
| 604 | UGAGAAAU U UACAGGAA | 84 | UUCCUGUA CUGAUGAGGCCGUUAGGCCGAA AUUUCUCA | 3861 |
| 605 | GAGAAAUU U ACAGGAAA | 85 | UUUCCUGU CUGAUGAGGCCGUUAGGCCGAA AAUUUCUC | 3862 |
| 606 | AGAAAUUU A CAGGAAAU | 86 | AUUUCCUG CUGAUGAGGCCGUUAGGCCGAA AAAUUUCU | 3863 |
| 615 | CAGGAAAU C CUGCAUGG | 87 | CCAUGCAG CUGAUGAGGCCGUUAGGCCGAA AUUUCCUG | 3864 |
| 635 | CGUGCGGU U CAGCAACA | 88 | UGUUGCUG CUGAUGAGGCCGUUAGGCCGAA ACCGCACG | 3865 |
| 636 | GUGCGGUU C AGCAACAA | 89 | UUGUUGCU CUGAUGAGGCCGUUAGGCCGAA AACCGCAC | 3866 |
| 672 | GAGAGCAU C CAGUGGCG | 90 | CGCCACUG CUGAUGAGGCCGUUAGGCCGAA AUGCUCUC | 3867 |
| 687 | CGGGACAU A GUCAGCAG | 91 | CUGCUGAC CUGAUGAGGCCGUUAGGCCGAA AUGUCCCG | 3868 |
| 690 | GACAUAGU C AGCAGUGA | 92 | UCACUGCU CUGAUGAGGCCGUUAGGCCGAA ACUAUGUC | 3869 |
| 701 | CAGUGACU U UCUCAGCA | 93 | UGCUGAGA CUGAUGAGGCCGUUAGGCCGAA AGUCACUG | 3870 |
| 702 | AGUGACUU U CUCAGCAA | 94 | UUGCUGAG CUGAUGAGGCCGUUAGGCCGAA AAGUCACU | 3871 |
| 703 | GUGACUUU C UCAGCAAC | 95 | GUUGCUGA CUGAUGAGGCCGUUAGGCCGAA AAAGUCAC | 3872 |
| 705 | GACUUUCU C AGCAACAU | 96 | AUGUUGCU CUGAUGAGGCCGUUAGGCCGAA AGAAAGUC | 3873 |
| 716 | CAACAUGU C GAUGGACU | 97 | AGUCCAUC CUGAUGAGGCCGUUAGGCCGAA ACAUGUUG | 3874 |
| 725 | GAUGGACU U CCAGAACC | 98 | GGUUCUGG CUGAUGAGGCCGUUAGGCCGAA AGUCCAUC | 3875 |
| 726 | AUGGACUU C CAGAACCA | 99 | UGGUUCUG CUGAUGAGGCCGUUAGGCCGAA AAGUCCAU | 3876 |
| 760 | AGUGUGAU C CAAGCUGU | 100 | ACAGCUUG CUGAUGAGGCCGUUAGGCCGAA AUCACACU | 3877 |
| 769 | CAAGCUGU C CCAAUGGG | 101 | CCCAUUGG CUGAUGAGGCCGUUAGGCCGAA ACAGCUUG | 3878 |
| 825 | ACCAAAAU C AUCUGUGC | 102 | GCACAGAU CUGAUGAGGCCGUUAGGCCGAA AUUUUGGU | 3879 |
| 828 | AAAAUCAU C UGUGCCCA | 103 | UGGGCACA CUGAUGAGGCCGUUAGGCCGAA AUGAUUUU | 3880 |
| 845 | GCAGUGCU C CGGGCGCU | 104 | AGCGCCCG CUGAUGAGGCCGUUAGGCCGAA AGCACUGC | 3881 |
| 866 | UGGCAAGU C CCCCAGUG | 105 | CACUGGGG CUGAUGAGGCCGUUAGGCCGAA ACUUGCCA | 3882 |
| 936 | UGCCUGGU C UGCCGCAA | 106 | UUGCGGCA CUGAUGAGGCCGUUAGGCCGAA ACCAGGCA | 3883 |
| 947 | CCGCAAAU U CCGAGACG | 107 | CGUCUCGG CUGAUGAGGCCGUUAGGCCGAA AUUUGCGG | 3884 |
| 948 | CGCAAAUU C CGAGACGA | 108 | UCGUCUCG CUGAUGAGGCCGUUAGGCCGAA AAUUUGCG | 3885 |
| 987 | CCCCCACU C AUGCUCUA | 109 | UAGAGCAU CUGAUGAGGCCGUUAGGCCGAA AGUGGGGG | 3886 |
| 993 | CUCAUGCU C UACAACCC | 110 | GGGUUGUA CUGAUGAGGCCGUUAGGCCGAA AGCAUGAG | 3887 |
| 995 | CAUGCUCU A CAACCCCA | 111 | UGGGGUUG CUGAUGAGGCCGUUAGGCCGAA AGAGCAUG | 3888 |
| 1010 | CACCACGU A CCAGAUGG | 112 | CCAUCUGG CUGAUGAGGCCGUUAGGCCGAA ACGUGGUG | 3889 |
| 1040 | GGGCAAAU A CAGCUUUG | 113 | CAAAGCUG CUGAUGAGGCCGUUAGGCCGAA AUUUGCCC | 3890 |
| 1046 | AUACAGCU U UGGUGCCA | 114 | UGGCACCA CUGAUGAGGCCGUUAGGCCGAA AGCUGUAU | 3891 |
| 1047 | UACAGCUU U GGUGCCAC | 115 | GUGGCACC CUGAUGAGGCCGUUAGGCCGAA AAGCUGUA | 3892 |
| 1072 | AGAAGUGU C CCCGUAAU | 116 | AUUACGGG CUGAUGAGGCCGUUAGGCCGAA ACACUUCU | 3893 |
| 1078 | GUCCCCGU A AUUAUGUG | 117 | CACAUAAU CUGAUGAGGCCGUUAGGCCGAA ACGGGGAC | 3894 |
| 1081 | CCCGUAAU U AUGUGGUG | 118 | CACCACAU CUGAUGAGGCCGUUAGGCCGAA AUUACGGG | 3895 |
| 1082 | CCGUAAUU A UGUGGUGA | 119 | UCACCACA CUGAUGAGGCCGUUAGGCCGAA AAUUACGG | 3896 |
| 1096 | UGACAGAU C ACGGCUCG | 120 | CGAGCCGU CUGAUGAGGCCGUUAGGCCGAA AUCUGUCA | 3897 |
| 1103 | UCACGGCU C GUGCGUCC | 121 | GGACGCAC CUGAUGAGGCCGUUAGGCCGAA AGCCGUGA | 3898 |
| 1110 | UCGUGCGU C CGAGCCUG | 122 | CAGGCUCG CUGAUGAGGCCGUUAGGCCGAA ACGCACGA | 3899 |
| 1133 | CGACAGCU A UGAGAUGG | 123 | CCAUCUCA CUGAUGAGGCCGUUAGGCCGAA AGCUGUCG | 3900 |
| 1155 | GACGGCGU C CGCAAGUG | 124 | CACUUGCG CUGAUGAGGCCGUUAGGCCGAA ACGCCGUC | 3901 |
| 1165 | GCAAGUGU A AGAAGUGC | 125 | GCACUUCU CUGAUGAGGCCGUUAGGCCGAA ACACUUGC | 3902 |
| 1183 | AAGGGCCU U GCCGCAAA | 126 | UUUGCGGC CUGAUGAGGCCGUUAGGCCGAA AGGCCCUU | 3903 |
| 1198 | AAGUGUGU A ACGGAAUA | 127 | UAUUCCGU CUGAUGAGGCCGUUAGGCCGAA ACACACUU | 3904 |
| 1206 | AACGGAAU A GGUAUUGG | 128 | CCAAUACC CUGAUGAGGCCGUUAGGCCGAA AUUCCGUU | 3905 |
| 1210 | GAAUAGGU A UUGGUGAA | 129 | UUCACCAA CUGAUGAGGCCGUUAGGCCGAA ACCUAUUC | 3906 |
| 1212 | AUAGGUAU U GGUGAAUU | 130 | AAUUCACC CUGAUGAGGCCGUUAGGCCGAA AUACCUAU | 3907 |
| 1220 | UGGUGAAU U UAAAGACU | 131 | AGUCUUUA CUGAUGAGGCCGUUAGGCCGAA AUUCACCA | 3908 |
| 1221 | GGUGAAUU U AAAGACUC | 132 | GAGUCUUU CUGAUGAGGCCGUUAGGCCGAA AAUUCACC | 3909 |
| 1222 | GUGAAUUU A AAGACUCA | 133 | UGAGUCUU CUGAUGAGGCCGUUAGGCCGAA AAAUUCAC | 3910 |
| 1229 | UAAAGACU C ACUCUCCA | 134 | UGGAGAGU CUGAUGAGGCCGUUAGGCCGAA AGUCUUUA | 3911 |
| 1233 | GACUCACU C UCCAUAAA | 135 | UUUAUGGA CUGAUGAGGCCGUUAGGCCGAA AGUGAGUC | 3912 |
| 1235 | CUCACUCU C CAUAAAUG | 136 | CAUUUAUG CUGAUGAGGCCGUUAGGCCGAA AGAGUGAG | 3913 |
| 1239 | CUCUCCAU A AAUGCUAC | 137 | GUAGCAUU CUGAUGAGGCCGUUAGGCCGAA AUGGAGAG | 3914 |
| 1246 | UAAAUGCU A CGAAUAUU | 138 | AAUAUUCG CUGAUGAGGCCGUUAGGCCGAA AGCAUUUA | 3915 |
| 1252 | CUACGAAU A UUAAACAC | 139 | GUGUUUAA CUGAUGAGGCCGUUAGGCCGAA AUUCGUAG | 3916 |
| 1254 | ACGAAUAU U AAACACUU | 140 | AAGUGUUU CUGAUGAGGCCGUUAGGCCGAA AUAUUCGU | 3917 |
| 1255 | CGAAUAUU A AACACUUC | 141 | GAAGUGUU CUGAUGAGGCCGUUAGGCCGAA AAUAUUCG | 3918 |
| 1262 | UAAACACU U CAAAAACU | 142 | AGUUUUUG CUGAUGAGGCCGUUAGGCCGAA AGUGUUUA | 3919 |
| 1263 | AAACACUU C AAAAACUG | 143 | CAGUUUUU CUGAUGAGGCCGUUAGGCCGAA AAGUGUUU | 3920 |
| 1277 | CUGCACCU C CAUCAGUG | 144 | CACUGAUG CUGAUGAGGCCGUUAGGCCGAA AGGUGCAG | 3921 |
| 1281 | ACCUCCAU C AGUGGCGA | 145 | UCGCCACU CUGAUGAGGCCGUUAGGCCGAA AUGGAGGU | 3922 |
| 1291 | GUGGCGAU C UCCACAUC | 146 | GAUGUGGA CUGAUGAGGCCGUUAGGCCGAA AUCGCCAC | 3923 |
| 1293 | GGCGAUCU C CACAUCCU | 147 | AGGAUGUG CUGAUGAGGCCGUUAGGCCGAA AGAUCGCC | 3924 |
| 1299 | CUCCACAU C CUGCCGGU | 148 | ACCGGCAG CUGAUGAGGCCGUUAGGCCGAA AUGUGGAG | 3925 |
| 1313 | GGUGGCAU U UAGGGGUG | 149 | CACCCCUA CUGAUGAGGCCGUUAGGCCGAA AUGCCACC | 3926 |
| 1314 | GUGGCAUU U AGGGGUGA | 150 | UCACCCCU CUGAUGAGGCCGUUAGGCCGAA AAUGCCAC | 3927 |
| 1315 | UGGCAUUU A GGGGUGAC | 151 | GUCACCCC CUGAUGAGGCCGUUAGGCCGAA AAAUGCCA | 3928 |
| 1325 | GGGUGACU C CUUCACAC | 152 | GUGUGAAG CUGAUGAGGCCGUUAGGCCGAA AGUCACCC | 3929 |
| 1328 | UGACUCCU U CACACAUA | 153 | UAUGUGUG CUGAUGAGGCCGUUAGGCCGAA AGGAGUCA | 3930 |
| 1329 | GACUCCUU C ACACAUAC | 154 | GUAUGUGU CUGAUGAGGCCGUUAGGCCGAA AAGGAGUC | 3931 |
| 1336 | UCACACAU A CUCCUCCU | 155 | AGGAGGAG CUGAUGAGGCCGUUAGGCCGAA AUGUGUGA | 3932 |
| 1339 | CACAUACU C CUCCUCUG | 156 | CAGAGGAG CUGAUGAGGCCGUUAGGCCGAA AGUAUGUG | 3933 |
| 1342 | AUACUCCU C CUCUGGAU | 157 | AUCCAGAG CUGAUGAGGCCGUUAGGCCGAA AGGAGUAU | 3934 |
| 1345 | CUCCUCCU C UGGAUCCA | 158 | UGGAUCCA CUGAUGAGGCCGUUAGGCCGAA AGGAGGAG | 3935 |
| 1351 | CUCUGGAU C CACAGGAA | 159 | UUCCUGUG CUGAUGAGGCCGUUAGGCCGAA AUCCAGAG | 3936 |
| 1366 | AACUGGAU A UUCUGAAA | 160 | UUUCAGAA CUGAUGAGGCCGUUAGGCCGAA AUCCAGUU | 3937 |
| 1368 | CUGGAUAU U CUGAAAAC | 161 | GUUUUCAG CUGAUGAGGCCGUUAGGCCGAA AUAUCCAG | 3938 |
| 1369 | UGGAUAUU C UGAAAACC | 162 | GGUUUUCA CUGAUGAGGCCGUUAGGCCGAA AAUAUCCA | 3939 |
| 1380 | AAAACCGU A AAGGAAAU | 163 | AUUUCCUU CUGAUGAGGCCGUUAGGCCGAA ACGGUUUU | 3940 |
| 1389 | AAGGAAAU C ACAGGGUU | 164 | AACCCUGU CUGAUGAGGCCGUUAGGCCGAA AUUUCCUU | 3941 |
| 1397 | CACAGGGU U UUUGCUGA | 165 | UCAGCAAA CUGAUGAGGCCGUUAGGCCGAA ACCCUGUG | 3942 |
| 1398 | ACAGGGUU U UUGCUGAU | 166 | AUCAGCAA CUGAUGAGGCCGUUAGGCCGAA AACCCUGU | 3943 |
| 1399 | CAGGGUUU U UGCUGAUU | 167 | AAUCAGCA CUGAUGAGGCCGUUAGGCCGAA AAACCCUG | 3944 |
| 1400 | AGGGUUUU U GCUGAUUC | 168 | GAAUCAGC CUGAUGAGGCCGUUAGGCCGAA AAAACCCU | 3945 |
| 1407 | UUGCUGAU U CAGGCUUG | 169 | CAAGCCUG CUGAUGAGGCCGUUAGGCCGAA AUCAGCAA | 3946 |
| 1408 | UGCUGAUU C AGGCUUGG | 170 | CCAAGCCU CUGAUGAGGCCGUUAGGCCGAA AAUCAGCA | 3947 |
| 1414 | UUCAGGCU U GGCCUGAA | 171 | UUCAGGCC CUGAUGAGGCCGUUAGGCCGAA AGCCUGAA | 3948 |
| 1437 | ACGGACCU C CAUGCCUU | 172 | AAGGCAUG CUGAUGAGGCCGUUAGGCCGAA AGGUCCGU | 3949 |
| 1445 | CCAUGCCU U UGAGAACC | 173 | GGUUCUCA CUGAUGAGGCCGUUAGGCCGAA AGGCAUGG | 3950 |
| 1446 | CAUGCCUU U GAGAACCU | 174 | AGGUUCUC CUGAUGAGGCCGUUAGGCCGAA AAGGCAUG | 3951 |
| 1455 | GAGAACCU A GAAAUCAU | 175 | AUGAUUUC CUGAUGAGGCCGUUAGGCCGAA AGGUUCUC | 3952 |
| 1461 | CUAGAAAU C AUACGCGG | 176 | CCGCGUAU CUGAUGAGGCCGUUAGGCCGAA AUUUCUAG | 3953 |
| 1464 | GAAAUCAU A CGCGGCAG | 177 | CUGCCGCG CUGAUGAGGCCGUUAGGCCGAA AUGAUUUC | 3954 |
| 1489 | AACAUGGU C AGUUUUCU | 178 | AGAAAACU CUGAUGAGGCCGUUAGGCCGAA ACCAUGUU | 3955 |
| 1493 | UGGUCAGU U UUCUCUUG | 179 | CAAGAGAA CUGAUGAGGCCGUUAGGCCGAA ACUGACCA | 3956 |
| 1494 | GGUCAGUU U UCUCUUGC | 180 | GCAAGAGA CUGAUGAGGCCGUUAGGCCGAA AACUGACC | 3957 |
| 1495 | GUCAGUUU U CUCUUGCA | 181 | UGCAAGAG CUGAUGAGGCCGUUAGGCCGAA AAACUGAC | 3958 |
| 1496 | UCAGUUUU C UCUUGCAG | 182 | CUGCAAGA CUGAUGAGGCCGUUAGGCCGAA AAAACUGA | 3959 |
| 1498 | AGUUUUCU C UUGCAGUC | 183 | GACUGCAA CUGAUGAGGCCGUUAGGCCGAA AGAAAACU | 3960 |
| 1500 | UUUUCUCU U GCAGUCGU | 184 | ACGACUGC CUGAUGAGGCCGUUAGGCCGAA AGAGAAAA | 3961 |
| 1506 | CUUGCAGU C GUCAGCCU | 185 | AGGCUGAC CUGAUGAGGCCGUUAGGCCGAA ACUGCAAG | 3962 |
| 1509 | GCAGUCGU C AGCCUGAA | 186 | UUCAGGCU CUGAUGAGGCCGUUAGGCCGAA ACGACUGC | 3963 |
| 1521 | CUGAACAU A ACAUCCUU | 187 | AAGGAUGU CUGAUGAGGCCGUUAGGCCGAA AUGUUCAG | 3964 |
| 1526 | CAUAACAU C CUUGGGAU | 188 | AUCCCAAG CUGAUGAGGCCGUUAGGCCGAA AUGUUAUG | 3965 |
| 1529 | AACAUCCU U GGGAUUAC | 189 | GUAAUCCC CUGAUGAGGCCGUUAGGCCGAA AGGAUGUU | 3966 |
| 1535 | CUUGGGAU U ACGCUCCC | 190 | GGGAGCGU CUGAUGAGGCCGUUAGGCCGAA AUCCCAAG | 3967 |
| 1536 | UUGGGAUU A CGCUCCCU | 191 | AGGGAGCG CUGAUGAGGCCGUUAGGCCGAA AAUCCCAA | 3968 |
| 1541 | AUUACGCU C CCUCAAGG | 192 | CCUUGAGG CUGAUGAGGCCGUUAGGCCGAA AGCGUAAU | 3969 |
| 1545 | CGCUCCCU C AAGGAGAU | 193 | AUCUCCUU CUGAUGAGGCCGUUAGGCCGAA AGGGAGCG | 3970 |
| 1554 | AAGGAGAU A AGUGAUGG | 194 | CCAUCACU CUGAUGAGGCCGUUAGGCCGAA AUCUCCUU | 3971 |
| 1572 | GAUGUGAU A AUUUCAGG | 195 | CCUGAAAU CUGAUGAGGCCGUUAGGCCGAA AUCACAUC | 3972 |
| 1575 | GUGAUAAU U UCAGGAAA | 196 | UUUCCUGA CUGAUGAGGCCGUUAGGCCGAA AUUAUCAC | 3973 |
| 1576 | UGAUAAUU U CAGGAAAC | 197 | GUUUCCUG CUGAUGAGGCCGUUAGGCCGAA AAUUAUCA | 3974 |
| 1577 | GAUAAUUU C AGGAAACA | 198 | UGUUUCCU CUGAUGAGGCCGUUAGGCCGAA AAAUUAUC | 3975 |
| 1591 | ACAAAAAU U UGUGCUAU | 199 | AUAGCACA CUGAUGAGGCCGUUAGGCCGAA AUUUUUGU | 3976 |
| 1592 | CAAAAAUU U GUGCUAUG | 200 | CAUAGCAC CUGAUGAGGCCGUUAGGCCGAA AAUUUUUG | 3977 |
| 1598 | UUUGUGCU A UGCAAAUA | 201 | UAUUUGCA CUGAUGAGGCCGUUAGGCCGAA AGCACAAA | 3978 |
| 1606 | AUGCAAAU A CAAUAAAC | 202 | GUUUAUUG CUGAUGAGGCCGUUAGGCCGAA AUUUGCAU | 3979 |
| 1611 | AAUACAAU A AACUGGAA | 203 | UUCCAGUU CUGAUGAGGCCGUUAGGCCGAA AUUGUAUU | 3980 |
| 1628 | AAAACUGU U UGGGACCU | 204 | AGGUCCCA CUGAUGAGGCCGUUAGGCCGAA ACAGUUUU | 3981 |
| 1629 | AAACUGUU U GGGACCUC | 205 | GAGGUCCC CUGAUGAGGCCGUUAGGCCGAA AACAGUUU | 3982 |
| 1637 | UGGGACCU C CGGUCAGA | 206 | UCUGACCG CUGAUGAGGCCGUUAGGCCGAA AGGUCCCA | 3983 |
| 1642 | CCUCCGGU C AGAAAACC | 207 | GGUUUUCU CUGAUGAGGCCGUUAGGCCGAA ACCGGAGG | 3984 |
| 1656 | ACCAAAAU U AUAAGCAA | 208 | UUGCUUAU CUGAUGAGGCCGUUAGGCCGAA AUUUUGGU | 3985 |
| 1657 | CCAAAAUU A UAAGCAAC | 209 | GUUGCUUA CUGAUGAGGCCGUUAGGCCGAA AAUUUUGG | 3986 |
| 1659 | AAAAUUAU A AGCAACAG | 210 | CUGUUGCU CUGAUGAGGCCGUUAGGCCGAA AUAAUUUU | 3987 |
| 1701 | GGCCAGGU C UGCCAUGC | 211 | GCAUGGCA CUGAUGAGGCCGUUAGGCCGAA ACCUGGCC | 3988 |
| 1712 | CCAUGCCU U GUGCUCCC | 212 | GGGAGCAC CUGAUGAGGCCGUUAGGCCGAA AGGCAUGG | 3989 |
| 1718 | CUUGUGCU C CCCCGAGG | 213 | CCUCGGGG CUGAUGAGGCCGUUAGGCCGAA AGCACAAG | 3990 |
| 1758 | GACUGCGU C UCUUGCCG | 214 | CGGCAAGA CUGAUGAGGCCGUUAGGCCGAA ACGCAGUC | 3991 |
| 1760 | CUGCGUCU C UUGCCGGA | 215 | UCCGGCAA CUGAUGAGGCCGUUAGGCCGAA AGACGCAG | 3992 |
| 1762 | GCGUCUCU U GCCGGAAU | 216 | AUUCCGGC CUGAUGAGGCCGUUAGGCCGAA AGAGACGC | 3993 |
| 1773 | CGGAAUGU C AGCCGAGG | 217 | CCUCGGCU CUGAUGAGGCCGUUAGGCCGAA ACAUUCCG | 3994 |
| 1809 | UGCAAGCU U CUGGAGGG | 218 | CCCUCCAG CUGAUGAGGCCGUUAGGCCGAA AGCUUGCA | 3995 |
| 1810 | GCAAGCUU C UGGAGGGU | 219 | ACCCUCCA CUGAUGAGGCCGUUAGGCCGAA AAGCUUGC | 3996 |
| 1832 | AAGGGAGU U UGUGGAGA | 220 | UCUCCACA CUGAUGAGGCCGUUAGGCCGAA ACUCCCUU | 3997 |
| 1833 | AGGGAGUU U GUGGAGAA | 221 | UUCUCCAC CUGAUGAGGCCGUUAGGCCGAA AACUCCCU | 3998 |
| 1844 | GGAGAACU C UGAGUGCA | 222 | UGCACUCA CUGAUGAGGCCGUUAGGCCGAA AGUUCUCC | 3999 |
| 1854 | GAGUGCAU A CAGUGCCA | 223 | UGGCACUG CUGAUGAGGCCGUUAGGCCGAA AUGCACUC | 4000 |
| 1879 | GCCUGCCU C AGGCCAUG | 224 | CAUGGCCU CUGAUGAGGCCGUUAGGCCGAA AGGCAGGC | 4001 |
| 1893 | AUGAACAU C ACCUGCAC | 225 | GUGCAGGU CUGAUGAGGCCGUUAGGCCGAA AUGUUCAU | 4002 |
| 1924 | ACAACUGU A UCCAGUGU | 226 | ACACUGGA CUGAUGAGGCCGUUAGGCCGAA ACAGUUGU | 4003 |
| 1926 | AACUGUAU C CAGUGUGC | 227 | GCACACUG CUGAUGAGGCCGUUAGGCCGAA AUACAGUU | 4004 |
| 1940 | UGCCCACU A CAUUGACG | 228 | CGUCAAUG CUGAUGAGGCCGUUAGGCCGAA AGUGGGCA | 4005 |
| 1944 | CACUACAU U GACGGCCC | 229 | GGGCCGUC CUGAUGAGGCCGUUAGGCCGAA AUGUAGUG | 4006 |
| 1962 | CACUGCGU C AAGACCUG | 230 | CAGGUCUU CUGAUGAGGCCGUUAGGCCGAA ACGCAGUG | 4007 |
| 1983 | GCAGGAGU C AUGGGAGA | 231 | UCUCCCAU CUGAUGAGGCCGUUAGGCCGAA ACUCCUGC | 4008 |
| 2007 | ACCCUGGU C UGGAAGUA | 232 | UACUUCCA CUGAUGAGGCCGUUAGGCCGAA ACCAGGGU | 4009 |
| 2015 | CUGGAAGU A CGCAGACG | 233 | CGUCUGCG CUGAUGAGGCCGUUAGGCCGAA ACUUCCAG | 4010 |
| 2050 | UGUGCCAU C CAAACUGC | 234 | GCAGUUUG CUGAUGAGGCCGUUAGGCCGAA AUGGCACA | 4011 |
| 2063 | CUGCACCU A CGGAUGCA | 235 | UGCAUCCG CUGAUGAGGCCGUUAGGCCGAA AGGUGCAG | 4012 |
| 2083 | GGCCAGGU C UUGAAGGC | 236 | GCCUUCAA CUGAUGAGGCCGUUAGGCCGAA ACCUGGCC | 4013 |
| 2085 | CCAGGUCU U GAAGGCUG | 237 | CAGCCUUC CUGAUGAGGCCGUUAGGCCGAA AGACCUGG | 4014 |
| 2095 | AAGGCUGU C CAACGAAU | 238 | AUUCGUUG CUGAUGAGGCCGUUAGGCCGAA ACAGCCUU | 4015 |
| 2110 | AUGGGCCU A AGAUCCCG | 239 | CGGGAUCU CUGAUGAGGCCGUUAGGCCGAA AGGCCCAU | 4016 |
| 2115 | CCUAAGAU C CCGUCCAU | 240 | AUGGACGG CUGAUGAGGCCGUUAGGCCGAA AUCUUAGG | 4017 |
| 2120 | GAUCCCGU C CAUCGCCA | 241 | UGGCGAUG CUGAUGAGGCCGUUAGGCCGAA ACGGGAUC | 4018 |
| 2124 | CCGUCCAU C GCCACUGG | 242 | CCAGUGGC CUGAUGAGGCCGUUAGGCCGAA AUGGACGG | 4019 |
| 2148 | GGGGCCCU C CUCUUGCU | 243 | AGCAAGAG CUGAUGAGGCCGUUAGGCCGAA AGGGCCCC | 4020 |
| 2151 | GCCCUCCU C UUGCUGCU | 244 | AGCAGCAA CUGAUGAGGCCGUUAGGCCGAA AGGAGGGC | 4021 |
| 2153 | CCUCCUCU U GCUGCUGG | 245 | CCAGCAGC CUGAUGAGGCCGUUAGGCCGAA AGAGGAGG | 4022 |
| 2178 | CUGGGGAU C GGCCUCUU | 246 | AAGAGGCC CUGAUGAGGCCGUUAGGCCGAA AUCCCCAG | 4023 |
| 2184 | AUCGGCCU C UUCAUGCG | 247 | CGCAUGAA CUGAUGAGGCCGUUAGGCCGAA AGGCCGAU | 4024 |
| 2186 | CGGCCUCU U CAUGCGAA | 248 | UUCGCAUG CUGAUGAGGCCGUUAGGCCGAA AGAGGCCG | 4025 |
| 2187 | GGCCUCUU C AUGCGAAG | 249 | CUUCGCAU CUGAUGAGGCCGUUAGGCCGAA AAGAGGCC | 4026 |
| 2205 | CGCCACAU C GUUCGGAA | 250 | UUCCGAAC CUGAUGAGGCCGUUAGGCCGAA AUGUGGCG | 4027 |
| 2208 | CACAUCGU U CGGAAGCG | 251 | CGCUUCCG CUGAUGAGGCCGUUAGGCCGAA ACGAUGUG | 4028 |
| 2209 | ACAUCGUU C GGAAGCGC | 252 | GCGCUUCC CUGAUGAGGCCGUUAGGCCGAA AACGAUGU | 4029 |
| 2250 | AGGGAGCU U GUGGAGCC | 253 | GGCUCCAC CUGAUGAGGCCGUUAGGCCGAA AGCUCCCU | 4030 |
| 2260 | UGGAGCCU C UUACACCC | 254 | GGGUGUAA CUGAUGAGGCCGUUAGGCCGAA AGGCUCCA | 4031 |
| 2262 | GAGCCUCU U ACACCCAG | 255 | CUGGGUGU CUGAUGAGGCCGUUAGGCCGAA AGAGGCUC | 4032 |
| 2263 | AGCCUCUU A CACCCAGU | 256 | ACUGGGUG CUGAUGAGGCCGUUAGGCCGAA AAGAGGCU | 4033 |
| 2281 | GAGAAGCU C CCAACCAA | 257 | UUGGUUGG CUGAUGAGGCCGUUAGGCCGAA AGCUUCUC | 4034 |
| 2293 | ACCAAGCU C UCUUGAGG | 258 | CCUCAAGA CUGAUGAGGCCGUUAGGCCGAA AGCUUGGU | 4035 |
| 2295 | CAAGCUCU C UUGAGGAU | 259 | AUCCUCAA CUGAUGAGGCCGUUAGGCCGAA AGAGCUUG | 4036 |
| 2297 | AGCUCUCU U GAGGAUCU | 260 | AGAUCCUC CUGAUGAGGCCGUUAGGCCGAA AGAGAGCU | 4037 |
| 2304 | UUGAGGAU C UUGAAGGA | 261 | UCCUUCAA CUGAUGAGGCCGUUAGGCCGAA AUCCUCAA | 4038 |
| 2306 | GAGGAUCU U GAAGGAAA | 262 | UUUCCUUC CUGAUGAGGCCGUUAGGCCGAA AGAUCCUC | 4039 |
| 2321 | AACUGAAU U CAAAAAGA | 263 | UCUUUUUG CUGAUGAGGCCGUUAGGCCGAA AUUCAGUU | 4040 |
| 2322 | ACUGAAUU C AAAAAGAU | 264 | AUCUUUUU CUGAUGAGGCCGUUAGGCCGAA AAUUCAGU | 4041 |
| 2331 | AAAAAGAU C AAAGUGCU | 265 | AGCACUUU CUGAUGAGGCCGUUAGGCCGAA AUCUUUUU | 4042 |
| 2345 | GCUGGGCU C CGGUGCGU | 266 | ACGCACCG CUGAUGAGGCCGUUAGGCCGAA AGCCCAGC | 4043 |
| 2354 | CGGUGCGU U CGGCACGG | 267 | CCGUGCCG CUGAUGAGGCCGUUAGGCCGAA ACGCACCG | 4044 |
| 2355 | GGUGCGUU C GGCACGGU | 268 | ACCGUGCC CUGAUGAGGCCGUUAGGCCGAA AACGCACC | 4045 |
| 2366 | CACGGUGU A UAAGGGAC | 269 | GUCCCUUA CUGAUGAGGCCGUUAGGCCGAA ACACCGUG | 4046 |
| 2368 | CGGUGUAU A AGGGACUC | 270 | GAGUCCCU CUGAUGAGGCCGUUAGGCCGAA AUACACCG | 4047 |
| 2376 | AAGGGACU C UGGAUCCC | 271 | GGGAUCCA CUGAUGAGGCCGUUAGGCCGAA AGUCCCUU | 4048 |
| 2382 | CUCUGGAU C CCAGAAGG | 272 | CCUUCUGG CUGAUGAGGCCGUUAGGCCGAA AUCCAGAG | 4049 |
| 2400 | GAGAAAGU U AAAAUUCC | 273 | GGAAUUUU CUGAUGAGGCCGUUAGGCCGAA ACUUUCUC | 4050 |
| 2401 | AGAAAGUU A AAAUUCCC | 274 | GGGAAUUU CUGAUGAGGCCGUUAGGCCGAA AACUUUCU | 4051 |
| 2406 | GUUAAAAU U CCCGUCGC | 275 | GCGACGGG CUGAUGAGGCCGUUAGGCCGAA AUUUUAAC | 4052 |
| 2407 | UUAAAAUU C CCGUCGCU | 276 | AGCGACGG CUGAUGAGGCCGUUAGGCCGAA AAUUUUAA | 4053 |
| 2412 | AUUCCCGU C GCUAUCAA | 277 | UUGAUAGC CUGAUGAGGCCGUUAGGCCGAA ACGGGAAU | 4054 |
| 2416 | CCGUCGCU A UCAAGGAA | 278 | UUCCUUGA CUGAUGAGGCCGUUAGGCCGAA AGCGACGG | 4055 |
| 2418 | GUCGCUAU C AAGGAAUU | 279 | AAUUCCUU CUGAUGAGGCCGUUAGGCCGAA AUAGCGAC | 4056 |
| 2426 | CAAGGAAU U AAGAGAAG | 280 | CUUCUCUU CUGAUGAGGCCGUUAGGCCGAA AUUCCUUG | 4057 |
| 2427 | AAGGAAUU A AGAGAAGC | 281 | GCUUCUCU CUGAUGAGGCCGUUAGGCCGAA AAUUCCUU | 4058 |
| 2441 | AGCAACAU C UCCGAAAG | 282 | CUUUCGGA CUGAUGAGGCCGUUAGGCCGAA AUGUUGCU | 4059 |
| 2443 | CAACAUCU C CGAAAGCC | 283 | GGCUUUCG CUGAUGAGGCCGUUAGGCCGAA AGAUGUUG | 4060 |
| 2463 | AAGGAAAU C CUCGAUGA | 284 | UCAUCGAG CUGAUGAGGCCGUUAGGCCGAA AUUUCCUU | 4061 |
| 2466 | GAAAUCCU C GAUGAAGC | 285 | GCUUCAUC CUGAUGAGGCCGUUAGGCCGAA AGGAUUUC | 4062 |
| 2477 | UGAAGCCU A CGUGAUGG | 286 | CCAUCACG CUGAUGAGGCCGUUAGGCCGAA AGGCUUCA | 4063 |
| 2526 | CUGGGCAU C UGCCUCAC | 287 | GUGAGGCA CUGAUGAGGCCGUUAGGCCGAA AUGCCCAG | 4064 |
| 2532 | AUCUGCCU C ACCUCCAC | 288 | GUGGAGGU CUGAUGAGGCCGUUAGGCCGAA AGGCAGAU | 4065 |
| 2537 | CCUCACCU C CACCGUGC | 289 | GCACGGUG CUGAUGAGGCCGUUAGGCCGAA AGGUGAGG | 4066 |
| 2550 | GUGCAACU C AUCACGCA | 290 | UGCGUGAU CUGAUGAGGCCGUUAGGCCGAA AGUUGCAC | 4067 |
| 2553 | CAACUCAU C ACGCAGCU | 291 | AGCUGCGU CUGAUGAGGCCGUUAGGCCGAA AUGAGUUG | 4068 |
| 2562 | ACGCAGCU C AUGCCCUU | 292 | AAGGGCAU CUGAUGAGGCCGUUAGGCCGAA AGCUGCGU | 4069 |
| 2570 | CAUGCCCU U CGGCUGCC | 293 | GGCAGCCG CUGAUGAGGCCGUUAGGCCGAA AGGGCAUG | 4070 |
| 2571 | AUGCCCUU C GGCUGCCU | 294 | AGGCAGCC CUGAUGAGGCCGUUAGGCCGAA AAGGGCAU | 4071 |
| 2580 | GGCUGCCU C CUGGACUA | 295 | UAGUCCAG CUGAUGAGGCCGUUAGGCCGAA AGGCAGCC | 4072 |
| 2588 | CCUGGACU A UGUCCGGG | 296 | CCCGGACA CUGAUGAGGCCGUUAGGCCGAA AGUCCAGG | 4073 |
| 2592 | GACUAUGU C CGGGAACA | 297 | UGUUCCCG CUGAUGAGGCCGUUAGGCCGAA ACAUAGUC | 4074 |
| 2611 | AAGACAAU A UUGGCUCC | 298 | GGAGCCAA CUGAUGAGGCCGUUAGGCCGAA AUUGUCUU | 4075 |
| 2613 | GACAAUAU U GGCUCCCA | 299 | UGGGAGCC CUGAUGAGGCCGUUAGGCCGAA AUAUUGUC | 4076 |
| 2618 | UAUUGGCU C CCAGUACC | 300 | GGUACUGG CUGAUGAGGCCGUUAGGCCGAA AGCCAAUA | 4077 |
| 2624 | CUCCCAGU A CCUGCUCA | 301 | UGAGCAGG CUGAUGAGGCCGUUAGGCCGAA ACUGGGAG | 4078 |
| 2631 | UACCUGCU C AACUGGUG | 302 | CACCAGUU CUGAUGAGGCCGUUAGGCCGAA AGCAGGUA | 4079 |
| 2649 | GUGCAGAU C GCAAAGGG | 303 | CCCUUUGC CUGAUGAGGCCGUUAGGCCGAA AUCUGCAC | 4080 |
| 2666 | CAUGAACU A CUUGGAGG | 304 | CCUCCAAG CUGAUGAGGCCGUUAGGCCGAA AGUUCAUG | 4081 |
| 2669 | GAACUACU U GGAGGACC | 305 | GGUCCUCC CUGAUGAGGCCGUUAGGCCGAA AGUAGUUC | 4082 |
| 2680 | AGGACCGU C GCUUGGUG | 306 | CACCAAGC CUGAUGAGGCCGUUAGGCCGAA ACGGUCCU | 4083 |
| 2684 | CCGUCGCU U GGUGCACC | 307 | GGUGCACC CUGAUGAGGCCGUUAGGCCGAA AGCGACGG | 4084 |
| 2715 | AGGAACGU A CUGGUGAA | 308 | UUCACCAG CUGAUGAGGCCGUUAGGCCGAA ACGUUCCU | 4085 |
| 2739 | CAGCAUGU C AAGAUCAC | 309 | GUGAUCUU CUGAUGAGGCCGUUAGGCCGAA ACAUGCUG | 4086 |
| 2745 | GUCAAGAU C ACAGAUUU | 310 | AAAUCUGU CUGAUGAGGCCGUUAGGCCGAA AUCUUGAC | 4087 |
| 2752 | UCACAGAU U UUGGGCUG | 311 | CAGCCCAA CUGAUGAGGCCGUUAGGCCGAA AUCUGUGA | 4088 |
| 2753 | CACAGAUU U UGGGCUGG | 312 | CCAGCCCA CUGAUGAGGCCGUUAGGCCGAA AAUCUGUG | 4089 |
| 2754 | ACAGAUUU U GGGCUGGC | 313 | GCCAGCCC CUGAUGAGGCCGUUAGGCCGAA AAAUCUGU | 4090 |
| 2792 | GAAAGAAU A CCAUGCAG | 314 | CUGCAUGG CUGAUGAGGCCGUUAGGCCGAA AUUCUUUC | 4091 |
| 2818 | AAGUGCCU A UCAAGUGG | 315 | CCACUUGA CUGAUGAGGCCGUUAGGCCGAA AGGCACUU | 4092 |
| 2820 | GUGCCUAU C AAGUGGAU | 316 | AUCCACUU CUGAUGAGGCCGUUAGGCCGAA AUAGGCAC | 4093 |
| 2834 | GAUGGCAU U GGAAUCAA | 317 | UUGAUUCC CUGAUGAGGCCGUUAGGCCGAA AUGCCAUC | 4094 |
| 2840 | AUUGGAAU C AAUUUUAC | 318 | GUAAAAUU CUGAUGAGGCCGUUAGGCCGAA AUUCCAAU | 4095 |
| 2844 | GAAUCAAU U UUACACAG | 319 | CUGUGUAA CUGAUGAGGCCGUUAGGCCGAA AUUGAUUC | 4096 |
| 2845 | AAUCAAUU U UACACAGA | 320 | UCUGUGUA CUGAUGAGGCCGUUAGGCCGAA AAUUGAUU | 4097 |
| 2846 | AUCAAUUU U ACACAGAA | 321 | UUCUGUGU CUGAUGAGGCCGUUAGGCCGAA AAAUUGAU | 4098 |
| 2847 | UCAAUUUU A CACAGAAU | 322 | AUUCUGUG CUGAUGAGGCCGUUAGGCCGAA AAAAUUGA | 4099 |
| 2856 | CACAGAAU C UAUACCCA | 323 | UGGGUAUA CUGAUGAGGCCGUUAGGCCGAA AUUCUGUG | 4100 |
| 2858 | CAGAAUCU A UACCCACC | 324 | GGUGGGUA CUGAUGAGGCCGUUAGGCCGAA AGAUUCUG | 4101 |
| 2860 | GAAUCUAU A CCCACCAG | 325 | CUGGUGGG CUGAUGAGGCCGUUAGGCCGAA AUAGAUUC | 4102 |
| 2877 | AGUGAUGU C UGGAGCUA | 326 | UAGCUCCA CUGAUGAGGCCGUUAGGCCGAA ACAUCACU | 4103 |
| 2885 | CUGGAGCU A CGGGQUGA | 327 | UCACCCCG CUGAUGAGGCCGUUAGGCCGAA AGCUCCAG | 4104 |
| 2898 | GUGACCGU U UGGGAGUU | 328 | AACUCCCA CUGAUGAGGCCGUUAGGCCGAA ACGGUCAC | 4105 |
| 2899 | UGACCGUU U GGGAGUUG | 329 | CAACUCCC CUGAUGAGGCCGUUAGGCCGAA AACGGUCA | 4106 |
| 2906 | UUGGGAGU U GAUGACCU | 330 | AGGUCAUC CUGAUGAGGCCGUUAGGCCGAA ACUCCCAA | 4107 |
| 2915 | GAUGACCU U UGGAUCCA | 331 | UGGAUCCA CUGAUGAGGCCGUUAGGCCGAA AGGUCAUC | 4108 |
| 2916 | AUGACCUU U GGAUCCAA | 332 | UUGGAUCC CUGAUGAGGCCGUUAGGCCGAA AAGGUCAU | 4109 |
| 2921 | CUUUGGAU C CAAGCCAU | 333 | AUGGCUUG CUGAUGAGGCCGUUAGGCCGAA AUCCAAAG | 4110 |
| 2930 | CAAGCCAU A UGACGGAA | 334 | UUCCGUCA CUGAUGAGGCCGUUAGGCCGAA AUGGCUUG | 4111 |
| 2940 | GACGGAAU C CCUGCCAG | 335 | CUGGCAGG CUGAUGAGGCCGUUAGGCCGAA AUUCCGUC | 4112 |
| 2955 | AGCGAGAU C UCCUCCAU | 336 | AUGGAGGA CUGAUGAGGCCGUUAGGCCGAA AUCUCGCU | 4113 |
| 2957 | CGAGAUCU C CUCCAUCC | 337 | GGAUGGAG CUGAUGAGGCCGUUAGGCCGAA AGAUCUCG | 4114 |
| 2960 | GAUCUCCU C CAUCCUGG | 338 | CCAGGAUG CUGAUGAGGCCGUUAGGCCGAA AGGAGAUC | 4115 |
| 2964 | UCCUCCAU C CUGGAGAA | 339 | UUCUCCAG CUGAUGAGGCCGUUAGGCCGAA AUGGAGGA | 4116 |
| 2985 | GAACGCCU C CCUCAGCC | 340 | GGCUGAGG CUGAUGAGGCCGUUAGGCCGAA AGGCGUUC | 4117 |
| 2989 | GCCUCCCU C AGCCACCC | 341 | GGGUGGCU CUGAUGAGGCCGUUAGGCCGAA AGGGAGGC | 4118 |
| 3000 | CCACCCAU A UGUACCAU | 342 | AUGGUACA CUGAUGAGGCCGUUAGGCCGAA AUGGGUGG | 4119 |
| 3004 | CCAUAUGU A CCAUCGAU | 343 | AUCGAUGG CUGAUGAGGCCGUUAGGCCGAA ACAUAUGG | 4120 |
| 3009 | UGUACCAU C GAUGUCUA | 344 | UAGACAUC CUGAUGAGGCCGUUAGGCCGAA AUGGUACA | 4121 |
| 3015 | AUCGAUGU C UACAUGAU | 345 | AUCAUGUA CUGAUGAGGCCGUUAGGCCGAA ACAUCGAU | 4122 |
| 3017 | CGAUGUCU A CAUGAUCA | 346 | UGAUCAUG CUGAUGAGGCCGUUAGGCCGAA AGACAUCG | 4123 |
| 3024 | UACAUGAU C AUGGUCAA | 347 | UUGACCAU CUGAUGAGGCCGUUAGGCCGAA AUCAUGUA | 4124 |
| 3030 | AUCAUGGU C AAGUGCUG | 348 | CAGCACUU CUGAUGAGGCCGUUAGGCCGAA ACCAUGAU | 4125 |
| 3045 | UGGAUGAU A GACGCAGA | 349 | UCUGCGUC CUGAUGAGGCCGUUAGGCCGAA AUCAUCCA | 4126 |
| 3055 | ACGCAGAU A GUCGCCCA | 350 | UGGGCGAC CUGAUGAGGCCGUUAGGCCGAA AUCUGCGU | 4127 |
| 3058 | CAGAUAGU C GCCCAAAG | 351 | CUUUGGGC CUGAUGAGGCCGUUAGGCCGAA ACUAUCUG | 4128 |
| 3068 | CCCAAAGU U CCGUGAGU | 352 | ACUCACGG CUGAUGAGGCCGUUAGGCCGAA ACUUUGGG | 4129 |
| 3069 | CCAAAGUU C CGUGAGUU | 353 | AACUCACG CUGAUGAGGCCGUUAGGCCGAA AACUUUGG | 4130 |
| 3077 | CCGUGAGU U GAUCAUCG | 354 | CGAUGAUC CUGAUGAGGCCGUUAGGCCGAA ACUCACGG | 4131 |
| 3081 | GAGUUGAU C AUCGAAUU | 355 | AAUUCGAU CUGAUGAGGCCGUUAGGCCGAA AUCAACUC | 4132 |
| 3084 | UUGAUCAU C GAAUUCUC | 356 | GAGAAUUC CUGAUGAGGCCGUUAGGCCGAA AUGAUCAA | 4133 |
| 3089 | CAUCGAAU U CUCCAAAA | 357 | UUUUGGAG CUGAUGAGGCCGUUAGGCCGAA AUUCGAUG | 4134 |
| 3090 | AUCGAAUU C UCCAAAAU | 358 | AUUUUGGA CUGAUGAGGCCGUUAGGCCGAA AAUUCGAU | 4135 |
| 3092 | CGAAUUCU C CAAAAUGG | 359 | CCAUUUUG CUGAUGAGGCCGUUAGGCCGAA AGAAUUCG | 4136 |
| 3119 | CCAGCGCU A CCUUGUCA | 360 | UGACAAGG CUGAUGAGGCCGUUAGGCCGAA AGCGCUGG | 4137 |
| 3123 | CGCUACCU U GUCAUUCA | 361 | UGAAUGAC CUGAUGAGGCCGUUAGGCCGAA AGGUAGCG | 4138 |
| 3126 | UACCUUGU C AUUCAGGG | 362 | CCCUGAAU CUGAUGAGGCCGUUAGGCCGAA ACAAGGUA | 4139 |
| 3129 | CUUGUCAU U CAGGGGGA | 363 | UCCCCCUG CUGAUGAGGCCGUUAGGCCGAA AUGACAAG | 4140 |
| 3130 | UUGUCAUU C AGGGGGAU | 364 | AUCCCCCU CUGAUGAGGCCGUUAGGCCGAA AAUGACAA | 4141 |
| 3151 | GAAUGCAU U UGCCAAGU | 365 | ACUUGGCA CUGAUGAGGCCGUUAGGCCGAA AUGCAUUC | 4142 |
| 3152 | AAUGCAUU U GCCAAGUC | 366 | GACUUGGC CUGAUGAGGCCGUUAGGCCGAA AAUGCAUU | 4143 |
| 3160 | UGCCAAGU C CUACAGAC | 367 | GUCUGUAG CUGAUGAGGCCGUUAGGCCGAA ACUUGGCA | 4144 |
| 3163 | CAAGUCCU A CAGACUCC | 368 | GGAGUCUG CUGAUGAGGCCGUUAGGCCGAA AGGACUUG | 4145 |
| 3170 | UACAGACU C CAACUUCU | 369 | AGAAGUUG CUGAUGAGGCCGUUAGGCCGAA AGUCUGUA | 4146 |
| 3176 | CUCCAACU U CUACCGUG | 370 | CACGGUAG CUGAUGAGGCCGUUAGGCCGAA AGUUGGAG | 4147 |
| 3177 | UCCAACUU C UACCGUGC | 371 | GCACGGUA CUGAUGAGGCCGUUAGGCCGAA AAGUUGGA | 4148 |
| 3179 | CAACUUCU A CCGUGCCC | 372 | GGGCACGG CUGAUGAGGCCGUUAGGCCGAA AGAAGUUG | 4149 |
| 3233 | CGACGAGU A CCUCAUCC | 373 | GGAUGAGG CUGAUGAGGCCGUUAGGCCGAA ACUCGUCG | 4150 |
| 3237 | GAGUACCU C AUCCCACA | 374 | UGUGGGAU CUGAUGAGGCCGUUAGGCCGAA AGGUACUC | 4151 |
| 3240 | UACCUCAU C CCACAGCA | 375 | UGCUGUGG CUGAUGAGGCCGUUAGGCCGAA AUGAGGUA | 4152 |
| 3254 | GCAGGGCU U CUUCAGCA | 376 | UGCUGAAG CUGAUGAGGCCGUUAGGCCGAA AGCCCUGC | 4153 |
| 3255 | CAGGGCUU C UUCAGCAG | 377 | CUGCUGAA CUGAUGAGGCCGUUAGGCCGAA AAGCCCUG | 4154 |
| 3257 | GGGCUUCU U CAGCAGCC | 378 | GGCUGCUG CUGAUGAGGCCGUUAGGCCGAA AGAAGCCC | 4155 |
| 3258 | GGCUUCUU C AGCAGCCC | 379 | GGGCUGCU CUGAUGAGGCCGUUAGGCCGAA AAGAAGCC | 4156 |
| 3269 | CAGCCCCU C CACGUCAC | 380 | GUGACGUG CUGAUGAGGCCGUUAGGCCGAA AGGGGCUG | 4157 |
| 3275 | CUCCACGU C ACGGACUC | 381 | GAGUCCGU CUGAUGAGGCCGUUAGGCCGAA ACGUGGAG | 4158 |
| 3283 | CACGGACU C CCCUCCUG | 382 | CAGGAGGG CUGAUGAGGCCGUUAGGCCGAA AGUCCGUG | 4159 |
| 3288 | ACUCCCCU C CUGAGCUC | 383 | GACCUCAG CUGAUGAGGCCGUUAGGCCGAA AGGGGAGU | 4160 |
| 3296 | CCUGAGCU C UCUGAGUG | 384 | CACUCAGA CUGAUGAGGCCGUUAGGCCGAA AGCUCAGG | 4161 |
| 3298 | UGAGCUCU C UGAGUGCA | 385 | UGCACUCA CUGAUGAGGCCGUUAGGCCGAA AGAGCUCA | 4162 |
| 3319 | GCAACAAU U CCACCGUG | 386 | CACGGUGG CUGAUGAGGCCGUUAGGCCGAA AUUGUUGC | 4163 |
| 3320 | CAACAAUU C CACCGUGG | 387 | CCACGGUG CUGAUGAGGCCGUUAGGCCGAA AAUUGUUG | 4164 |
| 3331 | CCGUGGCU U GCAUUGAU | 388 | AUCAAUGC CUGAUGAGGCCGUUAGGCCGAA AGCCACGG | 4165 |
| 3336 | GCUUGCAU U GAUAGAAA | 389 | UUUCUAUC CUGAUGAGGCCGUUAGGCCGAA AUGCAAGC | 4166 |
| 3340 | GCAUUGAU A GAAAUGGG | 390 | CCCAUUUC CUGAUGAGGCCGUUAGGCCGAA AUCAAUGC | 4167 |
| 3361 | AAAGCUGU C CCAUCAAG | 391 | CUUGAUGG CUGAUGAGGCCGUUAGGCCGAA ACAGCUUU | 4168 |
| 3366 | UGUCCCAU C AAGGAAGA | 392 | UCUUCCUU CUGAUGAGGCCGUUAGGCCGAA AUGGGACA | 4169 |
| 3380 | AGACAGCU U CUUGCAGC | 393 | GCUGCAAG CUGAUGAGGCCGUUAGGCCGAA AGCUGUCU | 4170 |
| 3381 | GACAGCUU C UUGCAGCG | 394 | CGCUGCAA CUGAUGAGGCCGUUAGGCCGAA AAGCUGUC | 4171 |
| 3383 | CAGCUUCU U GCAGCGAU | 395 | AUCGCUGC CUGAUGAGGCCGUUAGGCCGAA AGAAGCUG | 4172 |
| 3392 | GCAGCGAU A CAGCUCAG | 396 | CUGAGCUG CUGAUGAGGCCGUUAGGCCGAA AUCGCUGC | 4173 |
| 3398 | AUACAGCU C AGACCCCA | 397 | UGGGGUCU CUGAUGAGGCCGUUAGGCCGAA AGCUGUAU | 4174 |
| 3416 | AGGCGCCU U GACUGAGG | 398 | CCUCAGUC CUGAUGAGGCCGUUAGGCCGAA AGGCGCCU | 4175 |
| 3432 | GACAGCAU A GACGACAC | 399 | GUGUCGUC CUGAUGAGGCCGUUAGGCCGAA AUGCUGUC | 4176 |
| 3443 | CGACACCU U CCUCCCAG | 400 | CUGGGAGG CUGAUGAGGCCGUUAGGCCGAA AGGUGUCG | 4177 |
| 3444 | GACACCUU C CUCCCAGU | 401 | ACUGGGAG CUGAUGAGGCCGUUAGGCCGAA AAGGUGUC | 4178 |
| 3447 | ACCUUCCU C CCAGUGCC | 402 | GGCACUGG CUGAUGAGGCCGUUAGGCCGAA AGGAAGGU | 4179 |
| 3461 | GCCUGAAU A CAUAAACC | 403 | GGUUUAUG CUGAUGAGGCCGUUAGGCCGAA AUUCAGGC | 4180 |
| 3465 | GAAUACAU A AACCAGUC | 404 | GACUGGUU CUGAUGAGGCCGUUAGGCCGAA AUGUAUUC | 4181 |
| 3473 | AAACCAGU C CGUUCCCA | 405 | UGGGAACG CUGAUGAGGCCGUUAGGCCGAA ACUGGUUU | 4182 |
| 3477 | CAGUCCGU U CCCAAAAG | 406 | CUUUUGGG CUGAUGAGGCCGUUAGGCCGAA ACGGACUG | 4183 |
| 3478 | AGUCCGUU C CCAAAAGG | 407 | CCUUUUGG CUGAUGAGGCCGUUAGGCCGAA AACGGACU | 4184 |
| 3497 | CGCUGGCU C UGUGCAGA | 408 | UCUGCACA CUGAUGAGGCCGUUAGGCCGAA AGCCAGCG | 4185 |
| 3508 | UGCAGAAU C CUGUCUAU | 409 | AUAGACAG CUGAUGAGGCCGUUAGGCCGAA AUUCUGCA | 4186 |
| 3513 | AAUCCUGU C UAUCACAA | 410 | UUGUGAUA CUGAUGAGGCCGUUAGGCCGAA ACAGGAUU | 4187 |
| 3515 | UCCUGUCU A UCACAAUC | 411 | GAUUGUGA CUGAUGAGGCCGUUAGGCCGAA AGACAGGA | 4188 |
| 3517 | CUGUCUAU C ACAAUCAG | 412 | CUGAUUGU CUGAUGAGGCCGUUAGGCCGAA AUAGACAG | 4189 |
| 3523 | AUCACAAU C AGCCUCUG | 413 | CAGAGGCU CUGAUGAGGCCGUUAGGCCGAA AUUGUGAU | 4190 |
| 3529 | AUCAGCCU C UGAACCCC | 414 | GGGGUUCA CUGAUGAGGCCGUUAGGCCGAA AGGCUGAU | 4191 |
| 3560 | CCCACACU A CCAGGACC | 415 | GGUCCUGG CUGAUGAGGCCGUUAGGCCGAA AGUGUGGG | 4192 |
| 3599 | CCCCGAGU A UCUCAACA | 416 | UGUUGAGA CUGAUGAGGCCGUUAGGCCGAA ACUCGGGG | 4193 |
| 3601 | CCGAGUAU C UCAACACU | 417 | AGUGUUGA CUGAUGAGGCCGUUAGGCCGAA AUACUCGG | 4194 |
| 3603 | GAGUAUCU C AACACUGU | 418 | ACAGUGUU CUGAUGAGGCCGUUAGGCCGAA AGAUACUC | 4195 |
| 3612 | AACACUGU C CAGCCCAC | 419 | GUGGGCUG CUGAUGAGGCCGUUAGGCCGAA ACAGUGUU | 4196 |
| 3627 | ACCUGUGU C AACAGCAC | 420 | GUGCUGUU CUGAUGAGGCCGUUAGGCCGAA ACACAGGU | 4197 |
| 3638 | CAGCACAU U CGACAGCC | 421 | GGCUGUCG CUGAUGAGGCCGUUAGGCCGAA AUGUGCUG | 4198 |
| 3639 | AGCACAUU C GACAGCCC | 422 | GGGCUGUC CUGAUGAGGCCGUUAGGCCGAA AAUGUGCU | 4199 |
| 3681 | CACCAAAU U AGCCUGGA | 423 | UCCAGGCU CUGAUGAGGCCGUUAGGCCGAA AUUUGGUG | 4200 |
| 3682 | ACCAAAUU A GCCUGGAC | 424 | GUCCAGGC CUGAUGAGGCCGUUAGGCCGAA AAUUUGGU | 4201 |
| 3701 | CCCUGACU A CCAGCAGG | 425 | CCUGCUGG CUGAUGAGGCCGUUAGGCCGAA AGUCAGGG | 4202 |
| 3713 | GCAGGACU U CUUUCCCA | 426 | UGGGAAAG CUGAUGAGGCCGUUAGGCCGAA AGUCCUGC | 4203 |
| 3714 | CAGGACUU C UUUCCCAA | 427 | UUGGGAAA CUGAUGAGGCCGUUAGGCCGAA AAGUCCUG | 4204 |
| 3716 | GGACUUCU U UCCCAAGG | 428 | CCUUGGGA CUGAUGAGGCCGUUAGGCCGAA AGAAGUCC | 4205 |
| 3717 | GACUUCUU U CCCAAGGA | 429 | UCCUUCGG CUGAUGAGGCCGUUAGGCCGAA AAGAAGUC | 4206 |
| 3718 | ACUUCUUU C CCAAGGAA | 430 | UUCCUUGG CUGAUGAGGCCGUUAGGCCGAA AAAGAAGU | 4207 |
| 3744 | AAUGGCAU C UUUAAGGG | 431 | CCCUUAAA CUGAUGAGGCCGUUAGGCCGAA AUGCCAUU | 4208 |
| 3746 | UGGCAUCU U UAAGGGCU | 432 | AGCCCUUA CUGAUGAGGCCGUUAGGCCGAA AGAUGCCA | 4209 |
| 3747 | GGCAUCUU U AAGGGCUC | 433 | GAGCCCUU CUGAUGAGGCCGUUAGGCCGAA AAGAUGCC | 4210 |
| 3748 | GCAUCUUU A AGGGCUCC | 434 | GGAGCCCU CUGAUGAGGCCGUUAGGCCGAA AAAGAUGC | 4211 |
| 3755 | UAAGGGCU C CACAGCUG | 435 | CAGCUGUG CUGAUGAGGCCGUUAGGCCGAA AGCCCUUA | 4212 |
| 3776 | UGCAGAAU A CCUAAGGG | 436 | CCCUUAGG CUGAUGAGGCCGUUAGGCCGAA AUUCUGCA | 4213 |
| 3780 | GAAUACCU A AGGGUCGC | 437 | GCGACCCU CUGAUGAGGCCGUUAGGCCGAA AGGUAUUC | 4214 |
| 3786 | CUAAGGGU C GCGCCACA | 438 | UGUGGCGC CUGAUGAGGCCGUUAGGCCGAA ACCCUUAG | 4215 |
| 3806 | CAGUGAAU U UAUUGGAG | 439 | CUCCAAUA CUGAUGAGGCCGUUAGGCCGAA AUUCACUG | 4216 |
| 3807 | AGUGAAUU U AUUGGAGC | 440 | GCUCCAAU CUGAUGAGGCCGUUAGGCCGAA AAUUCACU | 4217 |
| 3808 | GUGAAUUU A UUGGAGCA | 441 | UGCUCCAA CUGAUGAGGCCGUUAGGCCGAA AAAUUCAC | 4218 |
| 3810 | GAAUUUAU U GGAGCAUG | 442 | CAUGCUCC CUGAUGAGGCCGUUAGGCCGAA AUAAAUUC | 4219 |
| 3831 | CGGAGGAU A GUAUGAGC | 443 | GCUCAUAC CUGAUGAGGCCGUUAGGCCGAA AUCCUCCG | 4220 |
| 3834 | AGGAUAGU A UGAGCCCU | 444 | AGGGCUCA CUGAUGAGGCCGUUAGGCCGAA ACUAUCCU | 4221 |
| 3843 | UGAGCCCU A AAAAUCCA | 445 | UGGAUUUU CUGAUGAGGCCGUUAGGCCGAA AGGGCUCA | 4222 |
| 3849 | CUAAAAAU C CAGACUCU | 446 | AGAGUCUG CUGAUGAGGCCGUUAGGCCGAA AUUUUUAG | 4223 |
| 3856 | UCCAGACU C UUUCGAUA | 447 | UAUCGAAA CUGAUGAGGCCGUUAGGCCGAA AGUCUGGA | 4224 |
| 3858 | CAGACUCU U UCGAUACC | 448 | GGUAUCGA CUGAUGAGGCCGUUAGGCCGAA AGAGUCUG | 4225 |
| 3859 | AGACUCUU U CGAUACCC | 449 | GGGUAUCG CUGAUGAGGCCGUUAGGCCGAA AAGAGUCU | 4226 |
| 3860 | GACUCUUU C GAUACCCA | 450 | UGGGUAUC CUGAUGAGGCCGUUAGGCCGAA AAAGAGUC | 4227 |
| 3864 | CUUUCGAU A CCCAGGAC | 451 | GUCCUGGG CUGAUGAGGCCGUUAGGCCGAA AUCGAAAG | 4228 |
| 3888 | CAGCAGGU C CUCCAUCC | 452 | GGAUGGAG CUGAUGAGGCCGUUAGGCCGAA ACCUGCUG | 4229 |
| 3891 | CAGGUCCU C CAUCCCAA | 453 | UUGGGAUG CUGAUGAGGCCGUUAGGCCGAA AGGACCUG | 4230 |
| 3895 | UCCUCCAU C CCAACAGC | 454 | GCUGUUGG CUGAUGAGGCCGUUAGGCCGAA AUGGAGGA | 4231 |
| 3915 | GCCCGCAU U AGCUCUUA | 455 | UAAGAGCU CUGAUGAGGCCGUUAGGCCGAA AUGCGGGC | 4232 |
| 3916 | CCCGCAUU A GCUCUUAG | 456 | CUAAGAGC CUGAUGAGGCCGUUAGGCCGAA AAUGCGGG | 4233 |
| 3920 | CAUUAGCU C UUAGACCC | 457 | GGGUCUAA CUGAUGAGGCCGUUAGGCCGAA AGCUAAUG | 4234 |
| 3922 | UUAGCUCU U AGACCCAC | 458 | GUGGGUCU CUGAUGAGGCCGUUAGGCCGAA AGAGCUAA | 4235 |
| 3923 | UAGCUCUU A GACCCACA | 459 | UGUGGGUC CUGAUGAGGCCGUUAGGCCGAA AAGAGCUA | 4236 |
| 3939 | AGACUGGU U UUGCAACG | 460 | CGUUGCAA CUGAUGAGGCCGUUAGGCCGAA ACCAGUCU | 4237 |
| 3940 | GACUGGUU U UGCAACGU | 461 | ACGUUGCA CUGAUGAGGCCGUUAGGCCGAA AACCAGUC | 4238 |
| 3941 | ACUGCUUU U GCAACGUU | 462 | AACGUUGC CUGAUGAGGCCGUUAGGCCCAA AAACCAGU | 4239 |
| 3949 | UGCAACGU U UACACCGA | 463 | UCGGUGUA CUGAUGAGGCCGUUAGGCCGAA ACGUUGCA | 4240 |
| 3950 | GCAACGUU U ACACCGAC | 464 | GUCGGUGU CUGAUGAGGCCGUUAGGCCGAA AACGUUGC | 4241 |
| 3951 | CAACGUUU A CACCGACU | 465 | AGUCGGUG CUGAUGAGGCCGUUAGGCCGAA AAACGUUG | 4242 |
| 3960 | CACCGACU A GCCAGGAA | 466 | UUCCUGGC CUGAUGAGGCCGUUAGGCCGAA AGUCGGUG | 4243 |
| 3971 | CAGGAAGU A CUUCCACC | 467 | GGUGGAAG CUGAUGAGGCCGUUAGGCCGAA ACUUCCUG | 4244 |
| 3974 | GAAGUACU U CCACCUCG | 468 | CGAGGUGG CUGAUGAGGCCGUUAGGCCGAA AGUACUUC | 4245 |
| 3975 | AAGUACUU C CACCUCGG | 469 | CCGAGGUG CUGAUGAGGCCGUUAGGCCGAA AAGUACUU | 4246 |
| 3981 | UUCCACCU C GGGCACAU | 470 | AUGUGCCC CUGAUGAGGCCGUUAGGCCGAA AGGUGGAA | 4247 |
| 3990 | GGGCACAU U UUGGGAAG | 471 | CUUCCCAA CUGAUGAGGCCGUUAGGCCGAA AUGUGCCC | 4248 |
| 3991 | GGCACAUU U UGGGAAGU | 472 | ACUUCCCA CUGAUGAGGCCGUUAGGCCGAA AAUGUGCC | 4249 |
| 3992 | GCACAUUU U GGGAAGUU | 473 | AACUUCCC CUGAUGAGGCCGUUAGGCCGAA AAAUGUGC | 4250 |
| 4000 | UGGGAAGU U GCAUUCCU | 474 | AGGAAUGC CUGAUGAGGCCGUUAGGCCGAA ACUUCCCA | 4251 |
| 4005 | AGUUGCAU U CCUUUGUC | 475 | GACAAAGG CUGAUGAGGCCGUUAGGCCGAA AUGCAACU | 4252 |
| 4006 | GUUGCAUU C CUUUGUCU | 476 | AGACAAAG CUGAUGAGGCCGUUAGGCCGAA AAUGCAAC | 4253 |
| 4009 | GCAUUCCU U UGUCUUCA | 477 | UGAAGACA CUGAUGAGGCCGUUAGGCCGAA AGGAAUGC | 4254 |
| 4010 | CAUUCCUU U GUCUUCAA | 478 | UUGAAGAC CUGAUGAGGCCGUUAGGCCGAA AAGGAAUG | 4255 |
| 4013 | UCCUUUGU C UUCAAACU | 479 | AGUUUGAA CUGAUGAGGCCGUUAGGCCGAA ACAAAGGA | 4256 |
| 4015 | CUUUGUCU U CAAACUGU | 480 | ACAGUUUG CUGAUGAGGCCGUUAGGCCGAA AGACAAAG | 4257 |
| 4016 | UUUGUCUU C AAACUGUG | 481 | CACAGUUU CUGAUGAGGCCGUUAGGCCGAA AAGACAAA | 4258 |
| 4031 | UGAAGCAU U UACAGAAA | 482 | UUUCUGUA CUGAUGAGGCCGUUAGGCCGAA AUGCUUCA | 4259 |
| 4032 | GAAGCAUU U ACAGAAAC | 483 | GUUUCUGU CUGAUGAGGCCGUUAGGCCGAA AAUGCUUC | 4260 |
| 4033 | AAGCAUUU A CAGAAACG | 484 | CGUUUCUG CUGAUGAGGCCGUUAGGCCGAA AAAUGCUU | 4261 |
| 4045 | AAACGCAU C CAGCAAGA | 485 | UCUUGCUG CUGAUGAGGCCGUUAGGCCGAA AUGCGUUU | 4262 |
| 4056 | GCAAGAAU A UUGUCCCU | 486 | AGGGACAA CUGAUGAGGCCGUUAGGCCGAA AUUCUUGC | 4263 |
| 4058 | AAGAAUAU U GUCCCUUU | 487 | AAAGGGAC CUGAUGAGGCCGUUAGGCCGAA AUAUUCUU | 4264 |
| 4061 | AAUAUUGU C CCUUUGAG | 488 | CUCAAAGG CUGAUGAGGCCGUUAGGCCGAA ACAAUAUU | 4265 |
| 4065 | UUGUCCCU U UGAGCAGA | 489 | UCUGCUCA CUGAUGAGGCCGUUAGGCCGAA AGGGACAA | 4266 |
| 4066 | UGUCCCUU U GAGCAGAA | 490 | UUCUGCUC CUGAUGAGGCCGUUAGGCCGAA AAGGGACA | 4267 |
| 4077 | GCAGAAAU U UAUCUUUC | 491 | GAAAGAUA CUGAUGAGGCCGUUAGGCCGAA AUUUCUGC | 4268 |
| 4078 | CAGAAAUU U AUCUUUCA | 492 | UGAAAGAU CUGAUGAGGCCGUUAGGCCGAA AAUUUCUG | 4269 |
| 4079 | AGAAAUUU A UCUUUCAA | 493 | UUGAAAGA CUGAUGAGGCCGUUAGGCCGAA AAAUUUCU | 4270 |
| 4081 | AAAUUUAU C UUUCAAAG | 494 | CUUUGAAA CUGAUGAGGCCGUUAGGCCGAA AUAAAUUU | 4271 |
| 4083 | AUUUAUCU U UCAAAGAG | 495 | CUCUUUGA CUGAUGAGGCCGUUAGGCCGAA AGAUAAAU | 4272 |
| 4084 | UUUAUCUU U CAAAGAGG | 496 | CCUCUUUG CUGAUGAGGCCGUUAGGCCGAA AAGAUAAA | 4273 |
| 4085 | UUAUCUUU C AAAGAGGU | 497 | ACCUCUUU CUGAUGAGGCCGUUAGGCCGAA AAAGAUAA | 4274 |
| 4094 | AAAGAGGU A UAUUUGAA | 498 | UUCAAAUA CUGAUGAGGCCGUUAGGCCGAA ACCUCUUU | 4275 |
| 4096 | AGAGGUAU A UUUGAAAA | 499 | UUUUCAAA CUGAUGAGGCCGUUAGGCCGAA AUACCUCU | 4276 |
| 4098 | AGGUAUAU U UGAAAAAA | 500 | UUUUUUCA CUGAUGAGGCCGUUAGGCCGAA AUAUACCU | 4277 |
| 4099 | GGUAUAUU U GAAAAAAA | 501 | UUUUUUUC CUGAUGAGGCCGUUAGGCCGAA AAUAUACC | 4278 |
| 4118 | AAAAAAGU A UAUGUGAG | 502 | CUCACAUA CUGAUGAGGCCGUUAGGCCGAA ACUUUUUU | 4279 |
| 4120 | AAAAGUAU A UGUGAGGA | 503 | UCCUCACA CUGAUGAGGCCGUUAGGCCGAA AUACUUUU | 4280 |
| 4130 | GUGAGGAU U UUUAUUGA | 504 | UCAAUAAA CUGAUGAGGCCGUUAGGCCGAA AUCCUCAC | 4281 |
| 4131 | UGAGGAUU U UUAUUGAU | 505 | AUCAAUAA CUGAUGAGGCCGUUAGGCCGAA AAUCCUCA | 4282 |
| 4132 | GAGGAUUU U UAUUGAUU | 506 | AAUCAAUA CUGAUGAGGCCGUUAGGCCGAA AAAUCCUC | 4283 |
| 4133 | AGGAUUUU U AUUGAUUG | 507 | CAAUCAAU CUGAUGAGGCCGUUAGGCCGAA AAAAUCCU | 4284 |
| 4134 | GGAUUUUU A UUGAUUGG | 508 | CCAAUCAA CUGAUGAGGCCGUUAGGCCGAA AAAAAUCC | 4285 |
| 4136 | AUUUUUAU U GAUUGGGG | 509 | CCCCAAUC CUGAUGAGGCCGUUAGGCCGAA AUAAAAAU | 4286 |
| 4140 | UUAUUGAU U GGGGAUCU | 510 | AGAUCCCC CUGAUGAGGCCGUUAGGCCGAA AUCAAUAA | 4287 |
| 4147 | UUGGGGAU C UUGGAGUU | 511 | AACUCCAA CUGAUGAGGCCGUUAGGCCGAA AUCCCCAA | 4288 |
| 4149 | GGGGAUCU U GGAGUUUU | 512 | AAAACUCC CUGAUGAGGCCGUUAGGCCGAA AGAUCCCC | 4289 |
| 4155 | CUUGGAGU U UUUCAUUG | 513 | CAAUGAAA CUGAUGAGGCCGUUAGGCCGAA ACUCCAAG | 4290 |
| 4156 | UUGGAGUU U UUCAUUGU | 514 | ACAAUGAA CUGAUGAGGCCGUUAGGCCGAA AACUCCAA | 4291 |
| 4157 | UGGAGUUU U UCAUUGUC | 515 | GACAAUGA CUGAUGAGGCCGUUAGGCCGAA AAACUCCA | 4292 |
| 4158 | GGAGUUUU U CAUUGUCG | 516 | CGACAAUG CUGAUGAGGCCGUUAGGCCGAA AAAACUCC | 4293 |
| 4159 | GAGUUUUU C AUUGUCGC | 517 | GCGACAAU CUGAUGAGGCCGUUAGGCCGAA AAAAACUC | 4294 |
| 4162 | UUUUUCAU U GUCGCUAU | 518 | AUAGCGAC CUGAUGAGGCCGUUAGGCCGAA AUGAAAAA | 4295 |
| 4165 | UUCAUUGU C GCUAUUGA | 519 | UCAAUAGC CUGAUGAGGCCGUUAGGCCGAA ACAAUGAA | 4296 |
| 4169 | UUGUCGCU A UUGAUUUU | 520 | AAAAUCAA CUGAUGAGGCCGUUAGGCCGAA AGCGACAA | 4297 |
| 4171 | GUCGCUAU U GAUUUUUA | 521 | UAAAAAUC CUGAUGAGGCCGUUAGGCCGAA AUAGCGAC | 4298 |
| 4175 | CUAUUGAU U UUUACUUC | 522 | GAAGUAAA CUGAUGAGGCCGUUAGGCCGAA AUCAAUAG | 4299 |
| 4176 | UAUUGAUU U UUACUUCA | 523 | UGAAGUAA CUGAUGAGGCCGUUAGGCCGAA AAUCAAUA | 4300 |
| 4177 | AUUGAUUU U UACUUCAA | 524 | UUGAAGUA CUGAUGAGGCCGUUAGGCCGAA AAAUCAAU | 4301 |
| 4178 | UUGAUUUU U ACUUCAAU | 525 | AUUGAAGU CUGAUGAGGCCGUUAGGCCGAA AAAAUCAA | 4302 |
| 4179 | UGAUUUUU A CUUCAAUG | 526 | CAUUGAAG CUGAUGAGGCCGUUAGGCCGAA AAAAAUCA | 4303 |
| 4182 | UUUUUACU U CAAUGGGC | 527 | CCCCAUUG CUGAUGAGGCCGUUAGGCCGAA AGUAAAAA | 4304 |
| 4183 | UUUUACUU C AAUGGGCU | 528 | AGCCCAUU CUGAUGAGGCCGUUAGGCCGAA AAGUAAAA | 4305 |
| 4192 | AAUGGGCU C UUCCAACA | 529 | UGUUGGAA CUGAUGAGGCCGUUAGGCCGAA AGCCCAUU | 4306 |
| 4194 | UGGGCUCU U CCAACAAG | 530 | CUUGUUGG CUGAUGAGGCCGUUAGGCCGAA AGAGCCCA | 4307 |
| 4195 | GGGCUCUU C CAACAAGG | 531 | CCUUGUUG CUGAUGAGGCCGUUAGGCCGAA AAGAGCCC | 4308 |
| 4212 | AAGAAGCU U GCUGGUAG | 532 | CUACCAGC CUGAUGAGGCCGUUAGGCCGAA AGCUUCUU | 4309 |
| 4219 | UUGCUGGU A GCACUUGC | 533 | GCAAGUGC CUGAUGAGGCCGUUAGGCCGAA ACCAGCAA | 4310 |
| 4225 | GUAGCACU U GCUACCCU | 534 | AGGGUAGC CUGAUCAGGCCGUUAGGCCGAA ACUCCUAC | 4311 |
| 4229 | CACUUGCU A CCCUGAGU | 535 | ACUCAGGG CUGAUGAGGCCGUUAGGCCGAA AGCAAGUG | 4312 |
| 4238 | CCCUGAGU U CAUCCAGG | 536 | CCUGGAUG CUGAUGAGGCCGUUAGGCCGAA ACUCAGGG | 4313 |
| 4239 | CCUGAGUU C AUCCAGGC | 537 | GCCUGGAU CUGAUGAGGCCGUUAGGCCGAA AACUCAGG | 4314 |
| 4242 | GAGUUCAU C CAGGCCCA | 538 | UGGGCCUG CUGAUGAGGCCGUUAGGCCGAA AUGAACUC | 4315 |
| 4280 | CCACAAGU C UUCCAGAG | 539 | CUCUGGAA CUGAUGAGGCCGUUAGGCCGAA ACUUGUGG | 4316 |
| 4282 | ACAAGUCU U CCAGAGGA | 540 | UCCUCUGG CUGAUGAGGCCGUUAGGCCGAA AGACUUGU | 4317 |
| 4283 | CAAGUCUU C CAGAGGAU | 541 | AUCCUCUG CUGAUGAGGCCGUUAGGCCGAA AAGACUUG | 4318 |
| 4295 | AGGAUGCU U GAUUCCAG | 542 | CUGGAAUC CUGAUGAGGCCGUUAGGCCGAA AGCAUCCU | 4319 |
| 4299 | UGCUUGAU U CCAGUGGU | 543 | ACCACUGG CUGAUGAGGCCGUUAGGCCGAA AUCAAGCA | 4320 |
| 4300 | GCUUGAUU C CAGUGGUU | 544 | AACCACUG CUGAUGAGGCCGUUAGGCCGAA AAUCAAGC | 4321 |
| 4308 | CCAGUGGU U CUGCUUCA | 545 | UGAAGCAG CUGAUGAGGCCGUUAGGCCGAA ACCACUGG | 4322 |
| 4309 | CAGUGGUU C UGCUUCAA | 546 | UUGAAGCA CUGAUGAGGCCGUUAGGCCGAA AACCACUG | 4323 |
| 4314 | GUUCUGCU U CAAGGCUU | 547 | AAGCCUUG CUGAUGAGGCCGUUAGGCCGAA AGCAGAAC | 4324 |
| 4315 | UUCUGCUU C AAGGCUUC | 548 | GAAGCCUU CUGAUGAGGCCGUUAGGCCGAA AAGCAGAA | 4325 |
| 4322 | UCAAGGCU U CCACUGCA | 549 | UGCAGUGG CUGAUGAGGCCGUUAGGCCGAA AGCCUUGA | 4326 |
| 4323 | CAAGGCUU C CACUGCAA | 550 | UUGCAGUG CUGAUGAGGCCGUUAGGCCGAA AAGCCUUG | 4327 |
| 4338 | AAAACACU A AAGAUCCA | 551 | UGGAUCUU CUGAUGAGGCCGUUAGGCCGAA AGUGUUUU | 4328 |
| 4344 | CUAAAGAU C CAAGAAGG | 552 | CCUUCUUG CUGAUGAGGCCGUUAGGCCGAA AUCUUUAG | 4329 |
| 4356 | GAAGGCCU U CAUGGCCC | 553 | GGGCCAUG CUGAUGAGGCCGUUAGGCCGAA AGGCCUUC | 4330 |
| 4357 | AAGGCCUU C AUGGCCCC | 554 | GGGGCCAU CUGAUGAGGCCGUUAGGCCGAA AAGGCCUU | 4331 |
| 4378 | GGCCGGAU C GGUACUGU | 555 | ACAGUACC CUGAUGAGGCCGUUAGGCCGAA AUCCGGCC | 4332 |
| 4382 | GGAUCGGU A CUGUAUCA | 556 | UGAUACAG CUGAUGAGGCCGUUAGGCCGAA ACCGAUCC | 4333 |
| 4387 | GGUACUGU A UCAAGUCA | 557 | UGACUUGA CUGAUGAGGCCGUUAGGCCGAA ACAGUACC | 4334 |
| 4389 | UACUGUAU C AAGUCAUG | 558 | CAUGACUU CUGAUGAGGCCGUUAGGCCGAA AUACAGUA | 4335 |
| 4394 | UAUCAAGU C AUGGCAGG | 559 | CCUGCCAU CUGAUGAGGCCGUUAGGCCGAA ACUUGAUA | 4336 |
| 4404 | UGGCAGGU A CAGUAGGA | 560 | UCCUACUG CUGAUGAGGCCGUUAGGCCGAA ACCUGCCA | 4337 |
| 4409 | GGUACAGU A GGAUAAGC | 561 | GCUUAUCC CUGAUGAGGCCGUUAGGCCGAA ACUGUACC | 4338 |
| 4414 | AGUAGGAU A AGCCACUC | 562 | GAGUGGCU CUGAUGAGGCCGUUAGGCCGAA AUCCUACU | 4339 |
| 4422 | AAGCCACU C UGUCCCUU | 563 | AAGGGACA CUGAUGAGGCCGUUAGGCCGAA AGUGGCUU | 4340 |
| 4426 | CACUCUGU C CCUUCCUG | 564 | CAGGAAGG CUGAUGAGGCCGUUAGGCCGAA ACAGAGUG | 4341 |
| 4430 | CUGUCCCU U CCUGGGCA | 565 | UGCCCAGU CUGAUGAGGCCGUUAGGCCGAA AGGGACAG | 4342 |
| 4431 | UGUCCCUU C CUGGGCAA | 566 | UUGCCCAG CUGAUGAGGCCGUUAGGCCGAA AAGGGACA | 4343 |
| 4462 | GGAUGAAU U CUUCCUUA | 567 | UAAGGAAG CUGAUGAGGCCGUUAGGCCGAA AUUCAUCC | 4344 |
| 4463 | GAUGAAUU C UUCCUUAG | 568 | CUAAGGAA CUGAUGAGGCCGUUAGGCCGAA AAUUCAUC | 4345 |
| 4465 | UGAAUUCU U CCUUAGAC | 569 | GUCUAAGG CUGAUGAGGCCGUUAGGCCGAA AGAAUUCA | 4346 |
| 4466 | GAAUUCUU C CUUAGACU | 570 | AGUCUAAG CUGAUGAGGCCGUUAGGCCGAA AAGAAUUC | 4347 |
| 4469 | UUCUUCCU U AGACUUAC | 571 | GUAAGUCU CUGAUGAGGCCGUUAGGCCGAA AGGAAGAA | 4348 |
| 4470 | UCUUCCUU A GACUUACU | 572 | AGUAAGUC CUGAUGAGGCCGUUAGGCCGAA AAGGAAGA | 4349 |
| 4475 | CUUAGACU U ACUUUUGU | 573 | ACAAAAGU CUGAUGAGGCCGUUAGGCCGAA AGUCUAAG | 4350 |
| 4476 | UUAGACUU A CUUUUGUA | 574 | UACAAAAG CUGAUGAGGCCGUUAGGCCGAA AAGUCUAA | 4351 |
| 4479 | GACUUACU U UUGUAAAA | 575 | UUUUACAA CUGAUGAGGCCGUUAGGCCGAA AGUAAGUC | 4352 |
| 4480 | ACUUACUU U UGUAAAAA | 576 | UUUUUACA CUGAUGAGGCCGUUAGGCCGAA AAGUAAGU | 4353 |
| 4481 | CUUACUUU U GUAAAAAU | 577 | AUUUUUAC CUGAUGAGGCCGUUAGGCCGAA AAAGUAAG | 4354 |
| 4484 | ACUUUUGU A AAAAUGUC | 578 | GACAUUUU CUGAUGAGGCCGUUAGGCCGAA ACAAAAGU | 4355 |
| 4492 | AAAAAUGU C CCCACGGU | 579 | ACCGUGGG CUGAUGAGGCCGUUAGGCCGAA ACAUUUUU | 4356 |
| 4501 | CCCACGGU A CUUACUCC | 580 | GGAGUAAG CUGAUGAGGCCGUUAGGCCGAA ACCGUGGG | 4357 |
| 4504 | ACGGUACU U ACUCCCCA | 581 | UGGGGAGU CUGAUGAGGCCGUUAGGCCGAA AGUACCGU | 4358 |
| 4505 | CGGUACUU A CUCCCCAC | 582 | GUGGGGAG CUGAUGAGGCCGUUAGGCCGAA AAGUACCG | 4359 |
| 4508 | UACUUACU C CCCACUGA | 583 | UCAGUGGG CUGAUGAGGCCGUUAGGCCGAA AGUAAGUA | 4360 |
| 4529 | CCAGUGGU U UCCAGUCA | 584 | UGACUGGA CUGAUGAGGCCGUUAGGCCGAA ACCACUGG | 4361 |
| 4530 | CAGUGGUU U CCAGUCAU | 585 | AUGACUGG CUGAUGAGGCCGUUAGGCCGAA AACCACUG | 4362 |
| 4531 | AGUGGUUU C CAGUCAUG | 586 | CAUGACUG CUGAUGAGGCCGUUAGGCCGAA AAACCACU | 4363 |
| 4536 | UUUCCAGU C AUGAGCGU | 587 | ACGCUCAU CUGAUGAGGCCGUUAGGCCGAA ACUGGAAA | 4364 |
| 4545 | AUGAGCGU U AGACUGAC | 588 | GUCAGUCU CUGAUGAGGCCGUUAGGCCGAA ACGCUCAU | 4365 |
| 4546 | UGAGCGUU A GACUGACU | 589 | AGUCAGUC CUGAUGAGGCCGUUAGGCCGAA AACGCUCA | 4366 |
| 4555 | GACUGACU U GUUUGUCU | 590 | AGACAAAC CUGAUGAGGCCGUUAGGCCGAA AGUCAGUC | 4367 |
| 4558 | UGACUUGU U UGUCUUCC | 591 | GGAAGACA CUGAUGAGGCCGUUAGGCCGAA ACAAGUCA | 4368 |
| 4559 | GACUUGUU U GUCUUCCA | 592 | UGGAAGAC CUGAUGAGGCCGUUAGGCCGAA AACAAGUC | 4369 |
| 4562 | UUGUUUGU C UUCCAUUC | 593 | GAAUGGAA CUGAUGAGGCCGUUAGGCCGAA ACAAACAA | 4370 |
| 4564 | GUUUGUCU U CCAUUCCA | 594 | UGGAAUGG CUGAUGAGGCCGUUAGGCCGAA AGACAAAC | 4371 |
| 4565 | UUUGUCUU C CAUUCCAU | 595 | AUGGAAUG CUGAUGAGGCCGUUAGGCCGAA AAGACAAA | 4372 |
| 4569 | UCUUCCAU U CCAUUGUU | 596 | AACAAUGG CUGAUGAGGCCGUUAGGCCGAA AUGGAAGA | 4373 |
| 4570 | CUUCCAUU C CAUUGUUU | 597 | AAACAAUG CUGAUGAGGCCGUUAGGCCGAA AAUGGAAG | 4374 |
| 4574 | CAUUCCAU U GUUUUGAA | 598 | UUCAAAAC CUGAUGAGGCCGUUAGGCCGAA AUGGAAUG | 4375 |
| 4577 | UCCAUUGU U UUGAAACU | 599 | AGUUUCAA CUGAUGAGGCCGUUAGGCCGAA ACAAUGGA | 4376 |
| 4578 | CCAUUGUU U UGAAACUC | 600 | GAGUUUCA CUGAUGAGGCCGUUAGGCCGAA AACAAUGG | 4377 |
| 4579 | CAUUGUUU U GAAACUCA | 601 | UGAGUUUC CUGAUGAGGCCGUUAGGCCGAA AAACAAUG | 4378 |
| 4586 | UUGAAACU C AGUAUGCC | 602 | GGCAUACU CUGAUGAGGCCGUUAGGCCGAA AGUUUCAA | 4379 |
| 4590 | AACUCAGU A UGCCGCCC | 603 | GGGCGGCA CUGAUGAGGCCGUUAGGCCGAA ACUGAGUU | 4380 |
| 4603 | GCCCCUGU C UUGCUGUC | 604 | GACAGCAA CUGAUGAGGCCGUUAGGCCGAA ACAGGGGC | 4381 |
| 4605 | CCCUGUCU U GCUGUCAU | 605 | AUGACAGC CUGAUGAGGCCGUUAGGCCGAA AGACAGGG | 4382 |
| 4611 | CUUGCUGU C AUGAPAUC | 606 | GAUUUCAU CUGAUGAGGCCGUUAGGCCGAA ACAGCAAG | 4383 |
| 4619 | CAUGAAAU C AGCAAGAG | 607 | CUCUUGCU CUGAUGAGGCCGUUAGGCCGAA AUUUCAUG | 4384 |
| 4640 | UGACACAU C AAAUAAUA | 608 | UAUUAUUU CUGAUGAGGCCGUUAGGCCGAA AUGUGUCA | 4385 |
| 4645 | CAUCAAAU A AUAACUCG | 609 | CGAGUUAU CUGAUGAGGCCGUUAGGCCGAA AUUUGAUG | 4386 |
| 4648 | CAAAUAAU A ACUCGGAU | 610 | AUCCGAGU CUGAUGAGGCCGUUAGGCCGAA AUUAUUUG | 4387 |
| 4652 | UAAUAACU C GGAUUCCA | 611 | UGGAAUCC CUGAUGAGGCCGUUAGGCCGAA AGUUAUUA | 4388 |
| 4657 | ACUCGGAU U CCAGCCCA | 612 | UGGGCUGG CUGAUGAGGCCGUUAGGCCGAA AUCCGAGU | 4389 |
| 4658 | CUCGGAUU C CAGCCCAC | 613 | GUGGGCUG CUGAUGAGGCCGUUAGGCCGAA AAUCCGAG | 4390 |
| 4669 | GCCCACAU U GGAUUCAU | 614 | AUGAAUCC CUGAUGAGGCCGUUAGGCCGAA AUGUGGGC | 4391 |
| 4674 | CAUUGGAU U CAUCAGCA | 615 | UGCUGAUG CUGAUGAGGCCGUUAGGCCGAA AUCCAAUG | 4392 |
| 4675 | AUUGGAUU C AUCAGCAU | 616 | AUGCUGAU CUGAUGAGGCCGUUAGGCCGAA AAUCCAAU | 4393 |
| 4678 | GGAUUCAU C AGCAUUUG | 617 | CAAAUGCU CUGAUGAGGCCGUUAGGCCGAA AUGAAUCC | 4394 |
| 4684 | AUCAGCAU U UGGACCAA | 618 | CUGGUCCA CUGAUGAGGCCGUUAGGCCGAA AUGCUGAU | 4395 |
| 4685 | UCAGCAUU U GGACCAAU | 619 | AUUGGUCC CUGAUGAGGCCGUUAGGCCGAA AAUGCUGA | 4396 |
| 4694 | GGACCAAU A GCCCACAG | 620 | CUGUGGGC CUGAUGAGGCCGUUAGGCCGAA AUUGGUCC | 4397 |
| 4718 | UGUGGAAU A CCUAAGGA | 621 | UCCUUAGG CUGAUGAGGCCGUUAGGCCGAA AUUCCACA | 4398 |
| 4722 | GAAUACCU A AGGAUAAC | 622 | GUUAUCCU CUGAUGAGGCCGUUAGGCCGAA AGGUAUUC | 4399 |
| 4728 | CUAAGGAU A ACACCGCU | 623 | AGCGGUGU CUGAUGAGGCCGUUAGGCCGAA AUCCUUAG | 4400 |
| 4737 | ACACCGCU U UUGUUCUC | 624 | GAGAACAA CUGAUGAGGCCGUUAGGCCGAA AGCGGUGU | 4401 |
| 4738 | CACCGCUU U UGUUCUCG | 625 | CGAGAACA CUGAUGAGGCCGUUAGGCCGAA AAGCGGUG | 4402 |
| 4739 | ACCGCUUU U GUUCUCGC | 626 | GCGAGAAC CUGAUGAGGCCGUUAGGCCGAA AAAGCGGU | 4403 |
| 4742 | GCUUUUGU U CUCGCAAA | 627 | UUUGCGAG CUGAUGAGGCCGUUAGGCCGAA ACAAAAGC | 4404 |
| 4743 | CUUUUGUU C UCGCAAAA | 628 | UUUUGCGA CUGAUGAGGCCGUUAGGCCGAA AACAAAAG | 4405 |
| 4745 | UUUGUUCU C GCAAAAAC | 629 | GUUUUUGC CUGAUGAGGCCGUUAGGCCGAA AGAACAAA | 4406 |
| 4756 | AAAAACGU A UCUCCUAA | 630 | UUAGGAGA CUGAUGAGGCCGUUAGGCCGAA ACGUUUUU | 4407 |
| 4758 | AAACGUAU C UCCUAAUU | 631 | AAUUAGGA CUGAUGAGGCCGUUAGGCCGAA AUACGUUU | 4408 |
| 4760 | ACGUAUCU C CUAAUUUG | 632 | CAAAUUAG CUGAUGAGGCCGUUAGGCCGAA AGAUACGU | 4409 |
| 4763 | UAUCUCCU A AUUUGAGG | 633 | CCUCAAAU CUGAUGAGGCCGUUAGGCCGAA AGGAGAUA | 4410 |
| 4766 | CUCCUAAU U UGAGGCUC | 634 | GAGCCUCA CUGAUGAGGCCGUUAGGCCGAA AUUAGGAG | 4411 |
| 4767 | UCCUAAUU U GAGGCUCA | 635 | UGAGCCUC CUGAUGAGGCCGUUAGGCCGAA AAUUAGGA | 4412 |
| 4774 | UUGAGGCU C AGAUGAAA | 636 | UUUCAUCU CUGAUGAGGCCGUUAGGCCGAA AGCCUCAA | 4413 |
| 4788 | AAAUGCAU C AGGUCCUU | 637 | AAGGACCU CUGAUGAGGCCGUUAGGCCGAA AUGCAUUU | 4414 |
| 4793 | CAUCAGGU C CUUUGGGG | 638 | CCCCAAAG CUGAUGAGGCCGUUAGGCCGAA ACCUGAUG | 4415 |
| 4796 | CAGGUCCU U UGGGGCAU | 639 | AUGCCCCA CUGAUGAGGCCGUUAGGCCGAA AGGACCUG | 4416 |
| 4797 | AGGUCCUU U GGGGCAUA | 640 | UAUGCCCC CUGAUGAGGCCGUUAGGCCGAA AAGGACCU | 4417 |
| 4805 | UGGGGCAU A GAUCAGAA | 641 | UUCUGAUC CUGAUGAGGCCGUUAGGCCGAA AUGCCCCA | 4418 |
| 4809 | GCAUAGAU C AGAAGACU | 642 | AGUCUUCU CUGAUGAGGCCGUUAGGCCGAA AUCUAUGC | 4419 |
| 4818 | AGAAGACU A CAAAAAUG | 643 | CAUUUUUG CUGAUGAGGCCGUUAGGCCGAA AGUCUUCU | 4420 |
| 4835 | AAGCUGCU C UGAAAUCU | 644 | AGAUUUCA CUGAUGAGGCCGUUAGGCCGAA AGCAGCUU | 4421 |
| 4842 | UCUGAAAU C UCCUUUAG | 645 | CUAAAGGA CUGAUGAGGCCGUUAGGCCGAA AUUUCAGA | 4422 |
| 4844 | UGAAAUCU C CUUUAGCC | 646 | GGCUAAAG CUGAUGAGGCCGUUAGGCCGAA AGAUUUCA | 4423 |
| 4847 | AAUCUCCU U UAGCCAUC | 647 | GAUGGCUA CUGAUGAGGCCGUUAGGCCGAA AGGAGAUU | 4424 |
| 4848 | AUCUCCUU U AGCCAUCA | 648 | UGAUGGCU CUGAUGAGGCCGUUAGGCCGAA AAGGAGAU | 4425 |
| 4849 | UCUCCUUU A GCCAUCAC | 649 | GUGAUGGC CUGAUGAGGCCGUUAGGCCGAA AAAGGAGA | 4426 |
| 4855 | UUAGCCAU C ACCCCAAC | 650 | GUUGGGGU CUGAUGAGGCCGUUAGGCCGAA AUGGCUAA | 4427 |
| 4874 | CCCAAAAU U AGUUUGUG | 651 | CACAAACU CUGAUGAGGCCGUUAGGCCGAA AUUUUGGG | 4428 |
| 4875 | CCAAAAUU A GUUUGUGU | 652 | ACACAAAC CUGAUGAGGCCGUUAGGCCGAA AAUUUUGG | 4429 |
| 4878 | AAAUUAGU U UGUGUUAC | 653 | GUAACACA CUGAUGAGGCCGUUAGGCCGAA ACUAAUUU | 4430 |
| 4879 | AAUUAGUU U GUGUUACU | 654 | AGUAACAC CUGAUGAGGCCGUUAGGCCGAA AACUAAUU | 4431 |
| 4884 | GUUUGUGU U ACUUAUGG | 655 | CCAUAAGU CUGAUGAGGCCGUUAGGCCGAA ACACAAAC | 4432 |
| 4885 | UUUGUGUU A CUUAUGGA | 656 | UCCAUAAG CUGAUGAGGCCGUUAGGCCGAA AACACAAA | 4433 |
| 4888 | GUGUUACU U AUGGAAGA | 657 | UCUUCCAU CUGAUGAGGCCGUUAGGCCGAA AGUAACAC | 4434 |
| 4889 | UGUUACUU A UGGAAGAU | 658 | AUCUUCCA CUGAUGAGGCCGUUAGGCCGAA AAGUAACA | 4435 |
| 4898 | UGGAAGAU A GUUUUCUC | 659 | GAGAAAAC CUGAUGAGGCCGUUAGGCCGAA AUCUUCCA | 4436 |
| 4901 | AAGAUAGU U UUCUCCUU | 660 | AAGGAGAA CUGAUGAGGCCGUUAGGCCGAA ACUAUCUU | 4437 |
| 4902 | AGAUAGUU U UCUCCUUU | 661 | AAAGGAGA CUGAUGAGGCCGUUAGGCCGAA AACUAUCU | 4438 |
| 4903 | GAUAGUUU U CUCCUUUU | 662 | AAAAGGAG CUGAUGAGGCCGUUAGGCCGAA AAACUAUC | 4439 |
| 4904 | AUAGUUUU C UCCUUUUA | 663 | UAAAAGGA CUGAUGAGGCCGUUAGGCCGAA AAAACUAU | 4440 |
| 4906 | AGUUUUCU C CUUUUACU | 664 | AGUAAAAG CUGAUGAGGCCGUUAGGCCGAA AGAAAACU | 4441 |
| 4909 | UUUCUCCU U UUACUUCA | 665 | UGAAGUAA CUGAUGAGGCCGUUAGGCCGAA AGGAGAAA | 4442 |
| 4910 | UUCUCCUU U UACUUCAC | 666 | GUGAAGUA CUGAUGAGGCCGUUAGGCCGAA AAGGAGAA | 4443 |
| 4911 | UCUCCUUU U ACUUCACU | 667 | AGUGAAGU CUGAUGAGGCCGUUAGGCCGAA AAAGGAGA | 4444 |
| 4912 | CUCCUUUU A CUUCACUU | 668 | AAGUGAAG CUGAUGAGGCCGUUAGGCCGAA AAAAGGAG | 4445 |
| 4915 | CUUUUACU U CACUUCAA | 669 | UUGAAGUG CUGAUGAGGCCGUUAGGCCGAA AGUAAAAG | 4446 |
| 4916 | UUUUACUU C ACUUCAAA | 670 | UUUGAAGU CUGAUGAGGCCGUUAGGCCGAA AAGUAAAA | 4447 |
| 4920 | ACUUCACU U CAAAAGCU | 671 | AGCUUUUG CUGAUGAGGCCGUUAGGCCGAA AGUGAAGU | 4448 |
| 4921 | CUUCACUU C AAAAGCUU | 672 | AAGCUUUU CUGAUGAGGCCGUUAGGCCGAA AAGUGAAG | 4449 |
| 4929 | CAAAAGCU U UUUACUCA | 673 | UGAGUAAA CUGAUGAGGCCGUUAGGCCGAA AGCUUUUG | 4450 |
| 4930 | AAAAGCUU U UUACUCAA | 674 | UUGAGUAA CUGAUGAGGCCGUUAGGCCGAA AAGCUUUU | 4451 |
| 4931 | AAAGCUUU U UACUCAAA | 675 | UUUGAGUA CUGAUGAGGCCGUUAGGCCGAA AAAGCUUU | 4452 |
| 4932 | AAGCUUUU U ACUCAAAG | 676 | CUUUGAGU CUGAUGAGGCCGUUAGGCCGAA AAAAGCUU | 4453 |
| 4933 | AGCUUUUU A CUCAAAGA | 677 | UCUUUGAG CUGAUGAGGCCGUUAGGCCGAA AAAAAGCU | 4454 |
| 4936 | UUUUUACU C AAAGAGUA | 678 | UACUCUUU CUGAUGAGGCCGUUAGGCCGAA ACUAAAAA | 4455 |
| 4944 | CAAAGAGU A UAUGUUCC | 679 | GGAACAUA CUGAUGAGGCCGUUAGGCCGAA ACUCUUUG | 4456 |
| 4946 | AAGAGUAU A UGUUCCCU | 680 | AGGGAACA CUGAUGAGGCCGUUAGGCCGAA AUACUCUU | 4457 |
| 4950 | GUAUAUGU U CCCUCCAG | 681 | CUGGAGGG CUGAUGAGGCCGUUAGGCCGAA ACAUAUAC | 4458 |
| 4951 | UAUAUGUU C CCUCCAGG | 682 | CCUGGAGG CUGAUGAGGCCGUUAGGCCGAA AACAUAUA | 4459 |
| 4955 | UGUUCCCU C CAGGUCAG | 683 | CUGACCUG CUGAUGAGGCCGUUAGGCCGAA AGGGAACA | 4460 |
| 4961 | CUCCAGGU C AGCUGCCC | 684 | GGGCAGCU CUGAUGAGGCCGUUAGGCCGAA ACCUGGAG | 4461 |
| 4981 | AACCCCCU C CUUACGCU | 685 | AGCGUAAG CUGAUGAGGCCGUUAGGCCGAA AGGGGGUU | 4462 |
| 4984 | CCCCUCCU U ACGCUUUG | 686 | CAAAGCGU CUGAUGAGGCCGUUAGGCCGAA AGGAGGGG | 4463 |
| 4985 | CCCUCCUU A CGCUUUGU | 687 | ACAAAGCG CUGAUGAGGCCGUUAGGCCGAA AAGGAGGG | 4464 |
| 4990 | CUUACGCU U UGUCACAC | 688 | GUGUGACA CUGAUGAGGCCGUUAGGCCGAA AGCGUAAG | 4465 |
| 4991 | UUACGCUU U GUCACACA | 689 | UGUGUGAC CUGAUGAGGCCGUUAGGCCGAA AAGCGUAA | 4466 |
| 4994 | CGCUUUGU C ACACAAAA | 690 | UUUUGUGU CUGAUGAGGCCGUUAGGCCGAA ACAAAGCG | 4467 |
| 5008 | AAAAGUGU C UCUGCCUU | 691 | AAGGCAGA CUGAUGAGGCCGUUAGGCCGAA ACACUUUU | 4468 |
| 5010 | AAGUGUCU C UGCCUUGA | 692 | UCAAGGCA CUGAUGAGGCCGUUAGGCCGAA AGACACUU | 4469 |
| 5016 | CUCUGCCU U GAGUCAUC | 693 | GAUGACUC CUGAUGAGGCCGUUAGGCCGAA AGGCAGAG | 4470 |
| 5021 | CCUUGAGU C AUCUAUUC | 694 | GAAUAGAU CUGAUGAGGCCGUUAGGCCGAA ACUCAAGG | 4471 |
| 5024 | UGAGUCAU C UAUUCAAG | 695 | CUUGAAUA CUGAUGAGGCCGUUAGGCCGAA AUGACUCA | 4472 |
| 5026 | AGUCAUCU A UUCAAGCA | 696 | UGCUUGAA CUGAUGAGGCCGUUAGGCCGAA AGAUGACU | 4473 |
| 5028 | UCAUCUAU U CAAGCACU | 697 | AGUGCUUG CUGAUGAGGCCGUUAGGCCGAA AUAGAUGA | 4474 |
| 5029 | CAUCUAUU C AAGCACUU | 698 | AAGUGCUU CUGAUGAGGCCGUUAGGCCGAA AAUAGAUG | 4475 |
| 5037 | CAAGCACU U ACAGCUCU | 699 | AGAGCUGU CUGAUGAGGCCGUUAGGCCGAA AGUGCUUG | 4476 |
| 5038 | AAGCACUU A CAGCUCUG | 700 | CAGAGCUG CUGAUGAGGCCGUUAGGCCGAA AAGUGCUU | 4477 |
| 5044 | UUACAGCU C UGGCCACA | 701 | UGUGGCCA CUGAUGAGGCCGUUAGGCCGAA AGCUGUAA | 4478 |
| 5062 | CAGGGCAU U UUACAGGU | 702 | ACCUGUAA CUGAUGAGGCCGUUAGGCCGAA AUGCCCUG | 4479 |
| 5063 | AGGGCAUU U UACAGGUG | 703 | CACCUGUA CUGAUGAGGCCGUUAGGCCGAA AAUGCCCU | 4480 |
| 5064 | GGGCAUUU U ACAGGUGC | 704 | GCACCUGU CUGAUGAGGCCGUUAGGCCGAA AAAUGCCC | 4481 |
| 5065 | GGCAUUUU A CAGGUGCG | 705 | CGCACCUG CUGAUGAGGCCGUUAGGCCGAA AAAAUGCC | 4482 |
| 5083 | AUGACAGU A GCAUUAUG | 706 | CAUAAUGC CUGAUGAGGCCGUUAGGCCGAA ACUGUCAU | 4483 |
| 5088 | AGUAGCAU U AUGAGUAG | 707 | CUACUCAU CUGAUGAGGCCGUUAGGCCGAA AUGCUACU | 4484 |
| 5089 | GUAGCAUU A UGAGUAGU | 708 | ACUACUCA CUGAUGAGGCCGUUAGGCCGAA AAUGCUAC | 4485 |
| 5095 | UUAUGAGU A GUGUGAAU | 709 | AUUCACAC CUGAUGAGGCCGUUAGGCCGAA ACUCAUAA | 4486 |
| 5104 | GUGUGAAU U CAGGUAGU | 710 | ACUACCUG CUGAUGAGGCCGUUAGGCCGAA AUUCACAC | 4487 |
| 5105 | UGUGAAUU C AGGUAGUA | 711 | UACUACCU CUGAUGAGGCCGUUAGGCCGAA AAUUCACA | 4488 |
| 5110 | AUUCAGGU A GUAAAUAU | 712 | AUAUUUAC CUGAUGAGGCCGUUAGGCCGAA ACCUGAAU | 4489 |
| 5113 | CAGGUAGU A AAUAUGAA | 713 | UUCAUAUU CUGAUGAGGCCGUUAGGCCGAA ACUACCUG | 4490 |
| 5117 | UAGUAAAU A UGAAACUA | 714 | UAGUUUCA CUGAUGAGGCCGUUAGGCCGAA AUUUACUA | 4491 |
| 5125 | AUGAAACU A GGGUUUGA | 715 | UCAAACCC CUGAUGAGGCCGUUAGGCCGAA AGUUUCAU | 4492 |
| 5130 | ACUAGGGU U UGAAAUUG | 716 | CAAUUUCA CUGAUGAGGCCGUUAGGCCGAA ACCCUAGU | 4493 |
| 5131 | CUAGGGUU U GAAAUUGA | 717 | UCAAUUUC CUGAUGAGGCCGUUAGGCCGAA AACCCUAG | 4494 |
| 5137 | UUUGAAAU U GAUAAUGC | 718 | GCAUUAUC CUGAUGAGGCCGUUAGGCCGAA AUUUCAAA | 4495 |
| 5141 | AAAUUGAU A AUGCUUUC | 719 | GAAAGCAU CUGAUGAGGCCGUUAGGCCGAA AUCAAUUU | 4496 |
| 5147 | AUAAUGCU U UCACAACA | 720 | UGUUGUGA CUGAUGAGGCCGUUAGGCCGAA AGCAUUAU | 4497 |
| 5148 | UAAUGCUU U CACAACAU | 721 | AUGUUGUG CUGAUGAGGCCGUUAGGCCGAA AAGCAUUA | 4498 |
| 5149 | AAUGCUUU C ACAACAUU | 722 | AAUGUUGU CUGAUGAGGCCGUUAGGCCGAA AAAGCAUU | 4499 |
| 5157 | CACAACAU U UGCAGAUG | 723 | CAUCUGCA CUGAUGAGGCCGUUAGGCCGAA AUGUUGUG | 4500 |
| 5158 | ACAACAUU U GCAGAUGU | 724 | ACAUCUGC CUGAUGAGGCCGUUAGGCCGAA AAUGUUGU | 4501 |
| 5167 | GCAGAUGU U UUAGAAGG | 725 | CCUUCUAA CUGAUGAGGCCGUUAGGCCGAA ACAUCUGC | 4502 |
| 5168 | CAGAUGUU U UAGAAGGA | 726 | UCCUUCUA CUGAUGAGGCCGUUAGGCCGAA AACAUCUG | 4503 |
| 5169 | AGAUGUUU U AGAAGGAA | 727 | UUCCUUCU CUGAUGAGGCCGUUAGGCCGAA AAACAUCU | 4504 |
| 5170 | GAUGUUUU A GAAGGAAA | 728 | UUUCCUUC CUGAUGAGGCCGUUAGGCCGAA AAAACAUC | 4505 |
| 5184 | AAAAAAGU U CCUUCCUA | 729 | UAGGAAGG CUGAUGAGGCCGUUAGGCCGAA ACUUUUUU | 4506 |
| 5185 | AAAAAGUU C CUUCCUAA | 730 | UUAGGAAG CUGAUGAGGCCGUUAGGCCGAA AACUUUUU | 4507 |
| 5188 | AAGUUCCU U CCUAAAAU | 731 | AUUUUAGG CUGAUGAGGCCGUUAGGCCGAA AGGAACUU | 4508 |
| 5189 | AGUUCCUU C CUAAAAUA | 732 | UAUUUUAG CUGAUGAGGCCGUUAGGCCGAA AAGGAACU | 4509 |
| 5192 | UCCUUCCU A AAAUAAUU | 733 | AAUUAUUU CUGAUGAGGCCGUUAGGCCGAA AGGAAGGA | 4510 |
| 5197 | CCUAAAAU A AUUUCUCU | 734 | AGAGAAAU CUGAUGAGGCCGUUAGGCCGAA AUUUUAGG | 4511 |
| 5200 | AAAAUAAU U UCUCUACA | 735 | UGUAGAGA CUGAUGAGGCCGUUAGGCCGAA AUUAUUUU | 4512 |
| 5201 | AAAUAAUU U CUCUACAA | 736 | UUGUAGAG CUGAUGAGGCCGUUAGGCCGAA AAUUAUUU | 4513 |
| 5202 | AAUAAUUU C UCUACAAU | 737 | AUUGUAGA CUGAUGAGGCCGUUAGGCCGAA AAAUUAUU | 4514 |
| 5204 | UAAUUUCU C UACAAUUG | 738 | CAAUUGUA CUGAUGAGGCCGUUAGGCCGAA AGAAAUUA | 4515 |
| 5206 | AUUUCUCU A CAAUUGGA | 739 | UCCAAUUG CUGAUGAGGCCGUUAGGCCGAA AGAGAAAU | 4516 |
| 5211 | UCUACAAU U GGAAGAUU | 740 | AAUCUUCC CUGAUGAGGCCGUUAGGCCGAA AUUGUAGA | 4517 |
| 5219 | UGGAAGAU U GGAAGAUU | 741 | AAUCUUCC CUGAUGAGGCCGUUAGGCCGAA AUCUUCCA | 4518 |
| 5227 | UGGAAGAU U CAGCUAGU | 742 | ACUAGCUG CUGAUGAGGCCGUUAGGCCGAA AUCUUCCA | 4519 |
| 5228 | GGAAGAUU C AGCUAGUU | 743 | AACUAGCU CUGAUGAGGCCGUUAGGCCGAA AAUCUUCC | 4520 |
| 5233 | AUUCAGCU A GUUAGGAG | 744 | CUCCUAAC CUGAUGAGGCCGUUAGGCCGAA AGCUGAAU | 4521 |
| 5236 | CAGCUAGU U AGGAGCCC | 745 | GGGCUCCU CUGAUGAGGCCGUUAGGCCGAA ACUAGCUG | 4522 |
| 5237 | AGCUAGUU A GGAGCCCA | 746 | UGGGCUCC CUGAUGAGGCCGUUAGGCCGAA AACUAGCU | 4523 |
| 5247 | GAGCCCAU U UUUUCCUA | 747 | UAGGAAAA CUGAUGAGGCCGUUAGGCCGAA AUGGGCUC | 4524 |
| 5248 | AGCCCAUU U UUUCCUAA | 748 | UUAGGAAA CUGAUGAGGCCGUUAGGCCGAA AAUGGGCU | 4525 |
| 5249 | GCCCAUUU U UUCCUAAU | 749 | AUUAGGAA CUGAUGAGGCCGUUAGGCCGAA AAAUGGGC | 4526 |
| 5250 | CCCAUUUU U UCCUAAUC | 750 | GAUGAGGA CUGAUGAGGCCGUUAGGCCGAA AAAAUGGG | 4527 |
| 5251 | CCAUUUUU U CCUAAUCU | 751 | AGAUUAGG CUGAUGAGGCCGUUAGGCCGAA AAAAAUGG | 4528 |
| 5252 | CAUUUUUU C CUAAUCUG | 752 | CAGAUGAG CUGAUGAGGCCGUUAGGCCGAA AAAAAAUG | 4529 |
| 5255 | UUUUUCCU A AUCUGUGU | 753 | ACACAGAU CUGAUGAGGCCGUUAGGCCGAA AGGAAAAA | 4530 |
| 5258 | UUCCUAAU C UGUGUGUG | 754 | CACACACA CUGAUGAGGCCGUUAGGCCGAA AUUAGGAA | 4531 |
| 5273 | UGCCCUGU A ACCUGACU | 755 | AGUCAGGU CUGAUGAGGCCGUUAGGCCGAA ACAGGGCA | 4532 |
| 5285 | UGACUGGU U AACAGCAG | 756 | CUGCUGUU CUGAUGAGGCCGUUAGGCCGAA ACCAGUCA | 4533 |
| 5286 | GACUGGUU A ACAGCAGU | 757 | ACUGCUGU CUGAUGAGGCCGUUAGGCCGAA AACCAGUC | 4534 |
| 5295 | ACAGCAGU C CUUUGUAA | 758 | UUACAAAG CUGAUGAGGCCGUUAGGCCGAA ACUGCUGU | 4535 |
| 5298 | GCAGUCCU U UGUAAACA | 759 | UGUUUACA CUGAUGAGGCCGUUAGGCCGAA AGGACUGC | 4536 |
| 5299 | CAGUCCUG U GUAAACAG | 760 | CUGUUUAC CUGAUGAGGCCGUUAGGCCGAA AAGGACUG | 4537 |
| 5302 | UCCUUUGU A AACAGUGU | 761 | ACACUGUU CUGAUGAGGCCGUUAGGCCGAA ACAAAGGA | 4538 |
| 5311 | AACAGUGU U UUAAACUC | 762 | GAGUUUAA CUGAUGAGGCCGUUAGGCCGAA ACACUGUU | 4539 |
| 5312 | ACAGUGUU U UAAACUCU | 763 | AGAGUUUA CUGAUGAGGCCGUUAGGCCGAA AACACUGU | 4540 |
| 5313 | CAGUGUUU U AAACUCUC | 764 | GAGAGUUU CUGAUGAGGCCGUUAGGCCGAA AAACACUG | 4541 |
| 5314 | AGUGUUUU A AACUCUCC | 765 | GGAGAGUU CUGAUGAGGCCGUUAGGCCGAA AAAACACU | 4542 |
| 5319 | UUUAAACU C UCCUAGUC | 766 | GACUAGGA CUGAUGAGGCCGUUAGGCCGAA AGUUUAAA | 4543 |
| 5321 | UAAACUCU C CUAGUCAA | 767 | UUGACUAG CUGAUGAGGCCGUUAGGCCGAA AGAGUUUA | 4544 |
| 5324 | ACUCUCCU A GUCAAUAU | 768 | AUAUUGAC CUGAUGAGGCCGUUAGGCCGAA AGGAGAGU | 4545 |
| 5327 | CUCCUAGU C AAUAUCCA | 769 | UGGAUAUU CUGAUGAGGCCGUUAGGCCGAA ACUAGGAG | 4546 |
| 5331 | UAGUCAAU A UCCACCCC | 770 | GGGGUGGA CUGAUGAGGCCGUUAGGCCGAA AUUGACUA | 4547 |
| 5333 | GUCAAUAU C CACCCCAU | 771 | AUGGGGUG CUGAUGAGGCCGUUAGGCCGAA AUAUUGAC | 4548 |
| 5342 | CACCCCAU C CAAUUUAU | 772 | AUAAAUUG CUGAUGAGGCCGUUAGGCCGAA AUGGGGUG | 4549 |
| 5347 | CAUCCAAU U UAUCAAGG | 773 | CCUUGAUA CUGAUGAGGCCGUUAGGCCGAA AUUGGAUG | 4550 |
| 5348 | AUCCAAUU U AUCAAGGA | 774 | UCCUUGAU CUGAUGAGGCCGUUAGGCCGAA AAUUGGAU | 4551 |
| 5349 | UCCAAUUU A UCAAGGAA | 775 | UUCCUUGA CUGAUGAGGCCGUUAGGCCGAA AAAUUGGA | 4552 |
| 5351 | CAAUUUAU C AAGGAAGA | 776 | UCUUCCUU CUGAUGAGGCCGUUAGGCCGAA AUAAAUUG | 4553 |
| 5366 | GAAAUGGU U CAGAAAAU | 777 | AUUUUCUG CUGAUGAGGCCGUUAGGCCGAA ACCAUUUC | 4554 |
| 5367 | AAAUGGUU C AGAAAAUA | 778 | UAUUUUCU CUGAUGAGGCCGUUAGGCCGAA AACCAUUU | 4555 |
| 5375 | CAGAAAAU A UUUUCAGC | 779 | GCUGAAAA CUGAUGAGGCCGUUAGGCCGAA AUUUUCUG | 4556 |
| 5377 | GAAAAUAU U UUCAGCCU | 780 | AGGCUGAA CUGAUGAGGCCGUUAGGCCGAA AUAUUUUC | 4557 |
| 5378 | AAAAUAUU U UCAGCCUA | 781 | UAGGCUGA CUGAUGAGGCCGUUAGGCCGAA AAUAUUUU | 4558 |
| 5379 | AAAUAUUU U CAGCCUAC | 782 | GUAGGCUG CUGAUGAGGCCGUUAGGCCGAA AAAUAUUU | 4559 |
| 5380 | AAUAUUUU C AGCCUACA | 783 | UGUAGGCU CUGAUGAGGCCGUUAGGCCGAA AAAAUAUU | 4560 |
| 5386 | UUCAGCCU A CAGUUAUG | 784 | CAUAACUG CUGAUGAGGCCGUUAGGCCGAA AGGCUGAA | 4561 |
| 5391 | CCUACAGU U AUGUUCAG | 785 | CUGAACAU CUGAUGAGGCCGUUAGGCCGAA ACUGUAGG | 4562 |
| 5392 | CUACAGUU A UGUUCAGU | 786 | ACUGAACA CUGAUGAGGCCGUUAGGCCGAA AACUGUAG | 4563 |
| 5396 | AGUUAUGU U CAGUCACA | 787 | UGUGACUG CUGAUGAGGCCGUUAGGCCGAA ACAUAACU | 4564 |
| 5397 | GUUAUGUU C AGUCACAC | 788 | GUGUGACU CUGAUGAGGCCGUUAGGCCGAA AACAUAAC | 4565 |
| 5401 | UGUUCAGU C ACACACAC | 789 | GUGUGUGU CUGAUGAGGCCGUUAGGCCGAA ACUGAACA | 4566 |
| 5412 | ACACACAU A CAAAAUGU | 790 | ACAUUUUG CUGAUGAGGCCGUUAGGCCGAA AUGUGUGU | 4567 |
| 5421 | CAAAAUGU U CCUUUUGC | 791 | GCAAAAGG CUGAUGAGGCCGUUAGGCCGAA ACAUUUUG | 4568 |
| 5422 | AAAAUGUU C CUUUUGCU | 792 | AGCAAAAG CUGAUGAGGCCGUUAGGCCGAA AACAUUUU | 4569 |
| 5425 | AUGUUCCU U UUGCUUUU | 793 | AAAAGCAA CUGAUGAGGCCGUUAGGCCGAA AGGAACAU | 4570 |
| 5426 | UGUUCCUU U UGCUUUUA | 794 | UAAAAGCA CUGAUGAGGCCGUUAGGCCGAA AAGGAACA | 4571 |
| 5427 | GUUCCUUU U GCUUUUAA | 795 | UUAAAAGC CUGAUGAGGCCGUUAGGCCGAA AAAGGAAC | 4572 |
| 5431 | CUUUUGCU U UUAAAGUA | 796 | UACUUUAA CUGAUGAGGCCGUUAGGCCGAA AGCAAAAG | 4573 |
| 5432 | UUUUGCUU U UAAAGUAA | 797 | UUACUUUA CUGAUGAGGCCGUUAGGCCGAA AAGCAAAA | 4574 |
| 5433 | UUUGCUUU U AAAGUAAU | 798 | AUUACUUU CUGAUGAGGCCGUUAGGCCGAA AAAGCAAA | 4575 |
| 5434 | UUGCUUUU A AAGUAAUU | 799 | AAUUACUU CUGAUGAGGCCGUUAGGCCGAA AAAAGCAA | 4576 |
| 5439 | UUUAAAGU A AUUUUUGA | 800 | UCAAAAAU CUGAUGAGGCCGUUAGGCCGAA ACUUUAAA | 4577 |
| 5442 | AAAGUAAU U UUUGACUC | 801 | GAGUCAAA CUGAUGAGGCCGUUAGGCCGAA AUUACUUU | 4578 |
| 5443 | AAGUAAUU U UUGACUCC | 802 | GGAGUCAA CUGAUGAGGCCGUUAGGCCGAA AAUUACUU | 4579 |
| 5444 | AGUAAUUU U UGACUCCC | 803 | GGGAGUCA CUGAUGAGGCCGUUAGGCCGAA AAAUUACU | 4580 |
| 5445 | GUAAUUUU U GACUCCCA | 804 | UGGGAGUC CUGAUGAGGCCGUUAGGCCGAA AAAAUUAC | 4581 |
| 5450 | UUUUGACU C CCAGAUCA | 805 | UGAUCUGG CUGAUGAGGCCGUUAGGCCGAA AGUCAAAA | 4582 |
| 5457 | UCCCAGAU C AGUCAGAG | 806 | CUCUGACU CUGAUGAGGCCGUUAGGCCGAA AUCUGGGA | 4583 |
| 5461 | AGAUCAGU C AGAGCCCC | 807 | GGGGCUCU CUGAUGAGGCCGUUAGGCCGAA ACUGAUCU | 4584 |
| 5471 | GAGCCCCU A CAGCAUUG | 808 | CAAUGCUG CUGAUGAGGCCGUUAGGCCGAA AGGGGCUC | 4585 |
| 5478 | UACAGCAU U GUUAAGAA | 809 | UUCUUAAC CUGAUGAGGCCGUUAGGCCGAA AUGCUGUA | 4586 |
| 5481 | AGCAUUGU U AAGAAAGU | 810 | ACUUUCUU CUGAUGAGGCCGUUAGGCCGAA ACAAUGCU | 4587 |
| 5482 | GCAUUGUU A AGAAAGUA | 811 | UACUUUCU CUGAUGAGGCCGUUAGGCCGAA AACAAUGC | 4588 |
| 5490 | AAGAAAGU A UUUGAUUU | 812 | AAAUCAAA CUGAUGAGGCCGUUAGGCCGAA ACUUUCUU | 4589 |
| 5492 | GAAAGUAU U UGAUUUUU | 813 | AAAAAUCA CUGAUGAGGCCGUUAGGCCGAA AUACUUUC | 4590 |
| 5493 | AAAGUAUU U GAUUUUUG | 814 | CAAAAAUC CUGAUGAGGCCGUUAGGCCGAA AAUACUUU | 4591 |
| 5497 | UAUUUGAU U UUUGUCUC | 815 | GAGACAAA CUGAUGAGGCCGUUAGGCCGAA AUCAAAUA | 4592 |
| 5498 | AUUUGAUU U UUGUCUCA | 816 | UGAGACAA CUGAUGAGGCCGUUAGGCCGAA AAUCAAAU | 4593 |
| 5499 | UUUGAUUU U UGUCUCAA | 817 | UUGAGACA CUGAUGAGGCCGUUAGGCCGAA AAAUCAAA | 4594 |
| 5500 | UUGAUUUU U GUCUCAAU | 818 | AUUGAGAC CUGAUGAGGCCGUUAGGCCGAA AAAAUCAA | 4595 |
| 5503 | AUUUUUGU C UCAAUGAA | 819 | UUCAUUGA CUGAUGAGGCCGUUAGGCCGAA ACAAAAAU | 4596 |
| 5505 | UUUUGUCU C AAUGAAAA | 820 | UUUUCAUU CUGAUGAGGCCGUUAGGCCGAA AGACAAAA | 4597 |
| 5515 | AUGAAAAU A AAACUAUA | 821 | UAUAGUUU CUGAUGAGGCCGUUAGGCCGAA AUUUUCAU | 4598 |
| 5521 | AUAAAACU A UAUUCAUU | 822 | AAUGAAUA CUGAUGAGGCCGUUAGGCCGAA AGUUUUAU | 4599 |
| 5523 | AAAACUAU A UUCAUUUC | 823 | GAAAUGAA CUGAUGAGGCCGUUAGGCCGAA AUAGUUUU | 4600 |

Input Sequence = NM_005228.1.
Cut Site = UH/.
Arm Length = 8.
Core Sequence = CUGAUGAG GCCGUUAGGC CGAA NM_005228.1 (Homo sapiens epidermal growth factor receptor (avian erythroblastic leukemia viral (v-erb-b) oncogene homolog) (EGFR), mRNA)

Underlined region can be any X sequence or linker, as described herein.

TABLE IV

Human EGFR Receptor Inozyme and Substrate Sequence

| | | | | |
|---|---|---|---|---|
| Pos | Substrate | Seq ID | Inozyme | Seq ID |

| | | | | |
|---|---|---|---|---|
| 13 | CGCUGCGC C GGAGUCCC | 824 | GGGACUCC CUGAUGAGGCCGUUAGGCCGAA ICGCAGCG | 4601 |
| 20 | CCGGAGUC C CGAGCUAG | 825 | CUAGCUCG CUGAUGAGGCCGUUAGGCCGAA IACUCCGG 4602 | |
| 21 | CGGAGUCC C GAGCUAGC | 826 | GCUAGCUC CUGAUGAGGCCGUUAGGCCCAA IGACUCCG | 4603 |
| 26 | UCCCGAGC U AGCCCCGG | 827 | CCGGGGCU CUGAUGAGGCCGUUAGGCCGAA ICUCGGGA | 4604 |
| 30 | GAGCUAGC C CCGGCGCC | 828 | GGCGCCGG CUGAUGAGGCCGUUAGGCCGAA ICUAGCUC | 4605 |
| 31 | AGCUAGCC C CGGCGCCG | 829 | CGGCGCCG CUGAUGAGGCCGUUAGGCCGAA IGCUAGCU | 4606 |
| 32 | GCUAGCCC C GGCGCCGC | 830 | GCGGCGCC CUGAUGAGGCCGUUAGGCCGAA IGGCUAGC | 4607 |
| 38 | CCCGGCGC C GCCGCCGC | 831 | GCGGCGGC CUGAUGAGGCCGUUAGGCCGAA ICGCCGGG | 4608 |
| 41 | GGCGCCGC C GCCGCCCA | 832 | UGGGCGGC CUGAUGAGGCCGUUAGGCCGAA ICGGCGCC | 4609 |
| 44 | GCCGCCGC C GCCCAGAC | 833 | GUCUGGGC CUGAUGAGGCCGUUAGGCCGAA ICGGCGGC | 4610 |
| 47 | GCCGCCGC C CAGACCGG | 834 | CCGGUCUG CUGAUGAGGCCGUUAGGCCGAA ICGGCGGC | 4611 |
| 48 | CCGCCGCC C AGACCGGA | 835 | UCCGGUCU CUGAUGAGGCCGUUAGGCCGAA IGCGGCGG | 4612 |
| 49 | CGCCGCCC A GACCGGAC | 836 | GUCCGGUC CUGAUGAGGCCGUUAGGCCGAA IGGCGGCG | 4613 |
| 53 | GCCCAGAC C GGACGACA | 837 | UGUCGUCC CUGAUGAGGCCGUUAGGCCGAA IUCUGGGC | 4614 |
| 61 | CGGACGAC A GGCCACCU | 838 | AGGUGGCC CUGAUGAGGCCGUUAGGCCGAA IUCGUCCG | 4615 |
| 65 | CGACAGGC C ACCUCGUC | 839 | GACGAGGU CUGAUGAGGCCGUUAGGCCGAA ICCUGUCG | 4616 |
| 66 | GACAGGCC A CCUCGUCG | 840 | CGACGAGG CUGAUGAGGCCGUUAGGCCGAA IGCCUGUC | 4617 |
| 68 | CAGGCCAC C UCGUCGGC | 841 | GCCGACGA CUGAUGAGGCCGUUAGGCCCGAA IUGGCCUG | 4618 |
| 69 | AGGCCACC U CGUCGGCG | 842 | CGCCGACG CUGAUGAGGCCGUUAGGCCGAA IGUGGCCU | 4619 |
| 80 | UCGGCGUC C GCCCGAGU | 843 | ACUCGGGC CUGAUGAGGCCGUUAGGCCGAA IACGCCGA | 4620 |
| 83 | GCGUCCGC C CGAGUCCC | 844 | GGGACUCG CUGAUGAGGCCGUUAGGCCGAA ICGGACGC | 4621 |
| 84 | CGUCCGCC C GAGUCCCC | 845 | GGGGACUC CUGAUGAGGCCGUUAGGCCGAA IGCGGACG | 4622 |
| 90 | CCCGAGUC C CCGCCUCG | 846 | CGAGGCGG CUGAUGAGGCCGUUAGGCCGAA IACUCGGG | 4623 |
| 91 | CCGAGUCC C CGCCUCGC | 847 | GCGAGGCG CUGAUGAGGCCGUUAGGCCGAA IGACUCGG | 4624 |
| 92 | CGAGUCCC C GCCUCGCC | 848 | GGCGAGGC CUGAUGAGGCCGUUAGGCCGAA IGGACUCG | 4625 |
| 95 | GUCCCCGC C UCGCCGCC | 849 | GGCGGCGA CUGAUGAGGCCGUUAGGCCGAA ICGGGGAC | 4626 |
| 96 | UCCCCGCC U CGCCGCCA | 850 | UGGCGGCG CUGAUGAGGCCGUUAGGCCGAA IGCGGGGA | 4627 |
| 100 | CGCCUCGC C GCCAACGC | 851 | GCGUUGGC CUGAUGAGGCCGUUAGGCCGAA ICGAGGCG | 4628 |
| 103 | CUCGCCGC C AACGCCAC | 852 | GUGGCGUU CUGAUGAGGCCGUUAGGCCGAA ICGGCGAG | 4629 |
| 104 | UCGCCGCC A ACGCCACA | 853 | UGUGGCGU CUGAUGAGGCCGUUAGGCCGAA IGCGGCGA | 4630 |
| 109 | GCCAACGC C ACAACCAC | 854 | GUGGUUGU CUGAUGAGGCCGUUAGGCCGAA ICGUUGGC | 4631 |
| 110 | CCAACGCC A CAACCACC | 855 | GGUGGUUG CUGAUGAGGCCGUUAGGCCGAA IGCGUUGG | 4632 |
| 112 | AACGCCAC A ACCACCGC | 856 | GCGGUGGU CUGAUGAGGCCGUUAGGCCGAA IUGGCGUU | 4633 |
| 115 | GCCACAAC C ACCGCGCA | 857 | UGCGCGGU CUGAUGAGGCCGUUAGGCCGAA IUUGUGGC | 4634 |
| 116 | CCACAACC A CCGCGCAC | 858 | GUGCGCGG CUGAUGAGGCCGUUAGGCCGAA IGUUGUGG | 4635 |
| 118 | ACAACCAC C GCGCACGG | 859 | CCGUGCGC CUGAUGAGGCCGUUAGGCCGAA IUGGUUGU | 4636 |
| 123 | CACCGCGC A CGGCCCCC | 860 | GGGGGCCG CUGAUGAGGCCGUUAGGCCGAA ICGCGGUG | 4637 |
| 128 | CGCACGGC C CCCUGACU | 861 | AGUCAGGG CUGAUGAGGCCGUUAGGCCGAA ICCGUGCG | 4638 |
| 129 | GCACGGCC C CCUGACUC | 862 | GAGUCAGG CUGAUGAGGCCGUUAGGCCGAA IGCCGUGC | 4639 |
| 130 | CACGGCCC C CUGACUCC | 863 | GGAGUCAG CUGAUGAGGCCGUUAGGCCGAA IGGCCGUG | 4640 |
| 131 | ACGGCCCC C UGACUCCG | 864 | CGGAGUCA CUGAUGAGGCCGUUAGGCCGAA IGGGCCGU | 4641 |
| 132 | CGGCCCCC U GACUCCGU | 865 | ACGGAGUC CUGAUGAGGCCGUUAGGCCGAA IGGGGCCG | 4642 |
| 136 | CCCCUGAC U CCGUCCAG | 866 | CUGGACGG CUGAUGAGGCCGUUAGGCCGAA IUCAGGGG | 4643 |
| 138 | CCUGACUC C GUCCAGUA | 867 | UACUGGAC CUGAUGAGGCCGUUAGGCCGAA IAGUCAGG | 4644 |
| 142 | ACUCCGUC C AGUAUUGA | 868 | UCAAUACU CUGAUGAGGCCGUUAGGCCGAA IACGGAGU | 4645 |
| 143 | CUCCGUCC A GUAUUGAU | 869 | AUCAAUAC CUGAUGAGGCCGUUAGGCCGAA IGACGGAG | 4646 |
| 161 | GGGAGAGC C GGAGCGAG | 870 | CUCGCUCC CUGAUGAGGCCGUUAGGCCGAA ICUCUCCC | 4647 |
| 171 | GAGCGAGC U CUUCGGGG | 871 | CCCCGAAG CUGAUGAGGCCGUUAGGCCGAA ICUCGCUC | 4648 |
| 173 | GCGAGCUC U UCGGGGAG | 872 | CUCCCCGA CUGAUGAGGCCGUUAGGCCGAA IAGCUCGC | 4649 |
| 183 | CGGGGAGC A GCGAUGCG | 873 | CGCAUCGC CUGAUGAGGCCGUUAGGCCGAA ICUCCCCG | 4650 |
| 194 | GAUGCGAC C CUCCGGGA | 874 | UCCCGGAG CUGAUGAGGCCGUUAGGCCGAA IUCGCAUC | 4651 |
| 195 | AUGCGACC C UCCGGGAC | 875 | GUCCCGGA CUGAUGAGGCCGUUAGGCCGAA IGUCGCAU | 4652 |
| 196 | UGCGACCC U CCGGGACG | 876 | CGUCCCGG CUGAUGAGGCCGUUAGGCCGAA IGGUCGCA | 4653 |
| 198 | CGACCCUC C GGGACGGC | 877 | GCCGUCCC CUGAUGAGGCCGUUAGGCCGAA IAGGGUCG | 4654 |
| 207 | GGGACGGC C GGGGCAGC | 878 | GCUGCCCC CUGAUGAGGCCGUUAGGCCGAA ICCGUCCC | 4655 |
| 213 | GCCGGGGC A GCGCUCCU | 879 | AGGAGCGC CUGAUGAGGCCGUUAGGCCGAA ICCCCGGC | 4656 |
| 218 | GGCAGCGC U CCUGGCGC | 880 | GCGCCAGG CUGAUGAGGCCGUUAGGCCGAA ICGCUGCC | 4657 |
| 220 | CAGCGCUC C UGGCGCUG | 881 | CAGCGCCA CUGAUGAGGCCGUUAGGCCGAA IAGCGCUG | 4658 |
| 221 | AGCGCUCC U GGCGCUGC | 882 | GCAGCGCC CUGAUGAGGCCGUUAGGCCGAA IGAGCGCU | 4659 |
| 227 | CCUGGCGC U GCUGGCUG | 883 | CAGCCAGC CUGAUGAGGCCGUUAGGCCGAA ICGCCAGG | 4660 |
| 230 | GGCGCUGC U GGCUGCGC | 884 | GCGCAGCC CUGAUGAGGCCGUUAGGCCGAA ICAGCGCC | 4661 |
| 234 | CUGCUGGC U GCGCUCUG | 885 | CAGAGCGC CUGAUGAGGCCGUUAGGCCGAA ICCAGCAG | 4662 |
| 239 | GGCUGCGC U CUGCCCGG | 886 | CCGGGCAG CUGAUGAGGCCGUUAGGCCGAA ICGCAGCC | 4663 |
| 241 | CUGCGCUC U GCCCGGCG | 887 | CGCCGGGC CUGAUGAGGCCGUUAGGCCGAA IAGCGCAG | 4664 |
| 244 | CGCUCUGC C CGGCGAGU | 888 | ACUCGCCG CUGAUGAGGCCGUUAGGCCGAA ICAGAGCG | 4665 |
| 245 | GCUCUGCC C GGCGAGUC | 889 | GACUCGCC CUGAUGAGGCCGUUAGGCCGAA IGCAGAGC | 4666 |
| 258 | AGUCGCGC U CUGGAGGA | 890 | UCCUCCAG CUGAUGAGGCCGUUAGGCCGAA ICCCGACU | 4667 |
| 260 | UCGGGCUC U GGAGGAAA | 891 | UUUCCUCC CUGAUGAGGCCGUUAGGCCGAA IAGCCCGA | 4668 |
| 280 | AAGUUUGC C AAGGCACG | 892 | CGUGCCUU CUGAUGAGGCCGUUAGGCCGAA ICAAACUU | 4669 |
| 281 | AGUUUGCC A AGGCACGA | 893 | UCGUGCCU CUGAUGAGGCCGUUAGGCCGAA IGCAAACU | 4670 |
| 286 | GCCAAGGC A CGAGUAAC | 894 | GUUACUCG CUGAUGAGGCCGUUAGGCCGAA ICCUUGGC | 4671 |
| 295 | CGAGUAAC A AGCUCACG | 895 | CGUGAGCU CUGAUGAGGCCGUUAGGCCGAA IUUACUCG | 4672 |
| 299 | UAACAAGC U CACGCAGU | 896 | ACUGCGUG CUGAUGAGGCCGUUAGGCCGAA ICUUGUUA | 4673 |
| 301 | ACAAGCUC A CGCAGUUG | 897 | CAACUGCG CUGAUGAGGCCGUUAGGCCGAA IAGCUUGU | 4674 |
| 305 | GCUCACGC A GUUGGGCA | 898 | UGCCCAAC CUGAUGAGGCCGUUAGGCCGAA ICGUGAGC | 4675 |
| 313 | AGUUGGGC A CUUUUGAA | 899 | UUCAAAAG CUGAUGAGGCCGUUAGGCCGAA ICCCAACU | 4676 |
| 315 | UUGGGCAC U UUUGAAGA | 900 | UCUUCAAA CUGAUGAGGCCGUUAGGCCGAA IUGCCCAA | 4677 |
| 326 | UGAAGAUC A UUUUCUCA | 901 | UGAGAAAA CUGAUGAGGCCGUUAGGCCGAA IAUCUUCA | 4678 |
| 332 | UCAUUUUC U CAGCCUCC | 902 | GGAGGCUG CUGAUGAGGCCGUUAGGCCGAA IAAAAUGA | 4679 |
| 334 | AUUUUCUC A GCCUCCAG | 903 | CUGGAGGC CUGAUGAGGCCGUUAGGCCGAA IAGAAAAU | 4680 |
| 337 | UUCUCAGC C UCCAGAGG | 904 | CCUCUGGA CUGAUGAGGCCGUUAGGCCGAA ICUGAGAA | 4681 |
| 338 | UCUCAGCC U CCAGAGGA | 905 | UCCUCUGG CUGAUGAGGCCGUUAGGCCGAA IGCUGAGA | 4682 |
| 340 | UCAGCCUC C AGAGGAUG | 906 | CAUCCUCU CUGAUGAGGCCGUUAGGCCGAA IAGGCUGA | 4683 |
| 341 | CAGCCUCC A GAGGAUGU | 907 | ACAUCCUC CUGAUGAGGCCGUUAGGCCGAA IGAGGCUG | 4684 |
| 352 | GGAUGUUC A AUAACUGU | 908 | ACAGUUAU CUGAUGAGGCCGUUAGGCCGAA IAACAUCC | 4685 |
| 358 | UCAAUAAC U GUGAGGUG | 909 | CACCUCAC CUGAUGAGGCCGUUAGGCCGAA IUUAUUGA | 4686 |
| 370 | AGGUGGUC C UUGGGAAU | 910 | AUUCCCAA CUGAUGAGGCCGUUAGGCCGAA IACCACCU | 4687 |
| 371 | GGUGGUCC U UGGGAAUU | 911 | AAUUCCCA CUGAUGAGGCCGUUAGGCCGAA IGACCACC | 4688 |
| 390 | GAAAUUAC C UAUGUGCA | 912 | UGCACAUA CUGAUGAGGCCGUUAGGCCGAA IUAAUUUC | 4689 |
| 391 | AAAUUACC U AUGUGCAG | 913 | CUGCACAU CUGAUGAGGCCGUUAGGCCGAA IGUAAUUU | 4690 |
| 398 | CUAUGUGC A GAGGAAUU | 914 | AAUUCCUC CUGAUGAGGCCGUUAGGCCGAA ICACAUAG | 4691 |
| 413 | UUAUGAUC U UUCCUUCU | 915 | AGAAGGAA CUGAUGAGGCCGUUAGGCCGAA IAUCAUAA | 4692 |
| 417 | GAUCUUUC C UUCUUAAA | 916 | UUUAAGAA CUGAUGAGGCCGUUAGGCCGAA IAAAGAUC | 4693 |
| 418 | AUCUUUCC U UCUUAAAG | 917 | CUUUAAGA CUGAUGAGGCCGUUAGGCCGAA IGAAAGAU | 4694 |
| 421 | UUUCCUUC U UAAAGACC | 918 | GGUCUUUA CUGAUGAGGCCGUUAGGCCGAA IAAGGAAA | 4695 |
| 429 | UUAAAGAC C AUCCAGGA | 919 | UCCUGGAU CUGAUGAGGCCGUUAGGCCGAA IUCUUUAA | 4696 |
| 430 | UAAAGACC A UCCAGGAG | 920 | CUCCUGGA CUGAUGAGGCCGUUAGGCCGAA IGUCUUUA | 4697 |
| 433 | AGACCAUC C AGGAGGUG | 921 | CACCUCCU CUGAUGAGGCCGUUAGGCCGAA IAUGGUCU | 4698 |
| 434 | GACCAUCC A GGAGGUGG | 922 | CCACCUCC CUGAUGAGGCCGUUAGGCCGAA IGAUGGUC | 4699 |
| 444 | GAGGUGGC U GGUUAUGU | 923 | ACAUAACC CUGAUGAGGCCGUUAGGCCGAA ICCACCUC | 4700 |
| 454 | GUUAUGUC C UCAUUGCC | 924 | GGCAAUGA CUGAUGAGGCCGUUAGGCCGAA IACAUAAC | 4701 |
| 455 | UUAUGUCC U CAUUGCCC | 925 | GGGCAAUG CUGAUGAGGCCGUUAGGCCGAA IGACAUAA | 4702 |
| 457 | AUGUCCUC A UUGCCCUC | 926 | GAGGGCAA CUGAUGAGGCCGUUAGGCCGAA IAGGACAU | 4703 |
| 462 | CUCAUUGC C CUCAACAC | 927 | GUGUUGAG CUGAUGAGGCCGUUAGGCCGAA ICAAUGAG | 4704 |
| 463 | UCAUUGCC C UCAACACA | 928 | UGUGUUGA CUGAUGAGGCCGUUAGGCCGAA IGCAAUGA | 4705 |
| 464 | CAUUGCCC U CAACACAG | 929 | CUGUGUUG CUGAUGAGGCCGUUAGGCCGAA IGGCAAUG | 4706 |
| 466 | UUGCCCUC A ACACAGUG | 930 | CACUGUGU CUGAUGAGGCCGUUAGGCCGAA IAGGGCAA | 4707 |
| 469 | CCCUCAAC A CAGUGGAG | 931 | CUCCACUG CUGAUGAGGCCGUUAGGCCGAA IUUGAGGG | 4708 |
| 471 | CUCAACAC A GUGGAGCG | 932 | CGCUCCAC CUGAUGAGGCCGUUAGGCCGAA IUGUUGAG | 4709 |
| 485 | GCGAAUUC C UUUGGAAA | 933 | UUUCCAAA CUGAUGAGGCCGUUAGGCCGAA IAAUUCGC | 4710 |
| 486 | CGAAUUCC U UUGGAAAA | 934 | UUUUCCAA CUGAUGAGGCCGUUAGGCCGAA IGAAUUCG | 4711 |
| 496 | UGGAAAAC C UGCAGAUC | 935 | GAUCUGCA CUGAUGAGGCCGUUAGGCCGAA IUUUUCCA | 4712 |
| 497 | GGAAAACC U GCAGAUCA | 936 | UGAUCUGC CUGAUGAGGCCGUUAGGCCGAA IGUUUUCC | 4713 |
| 500 | AAACCUGC A GAUCAUCA | 937 | UGAUGAUC CUGAUGAGGCCGUUAGGCCGAA ICAGGUUU | 4714 |
| 505 | UGCAGAUC A UCAGAGGA | 938 | UCCUCUGA CUGAUGAGGCCGUUAGGCCGAA IAUCUGCA | 4715 |
| 508 | AGAUCAUC A GAGGAAAU | 939 | AUUUCCUC CUGAUGAGGCCGUUAGGCCGAA IAUGAUCU | 4716 |
| 523 | AUAUGUAC U ACGAAAAU | 940 | AUUUUCGU CUGAUGAGGCCGUUAGGCCGAA IUACAUAU | 4717 |
| 534 | GAAAAUUC C UAUGCCUU | 941 | AAGGCAUA CUGAUGAGGCCGUUAGGCCGAA IAAUUUUC | 4718 |
| 535 | AAAAUUCC U AUGCCUUA | 942 | UAAGGCAU CUGAUGAGGCCGUUAGGCCGAA IGAAUUUU | 4719 |
| 540 | UCCUAUGC C UUAGCAGU | 943 | ACUGCUAA CUGAUGAGGCCGUUAGGCCGAA ICAUAGGA | 4720 |
| 541 | CCUAUGCC U UAGCAGUC | 944 | GACUGCUA CUGAUGAGGCCGUUAGGCCGAA IGCAUAGG | 4721 |
| 546 | GCCUUAGC A GUCUUAUC | 945 | GAUAAGAC CUGAUGAGGCCGUUAGGCCGAA ICUAAGGC | 4722 |
| 550 | UAGCAGUC U UAUCUAAC | 946 | GUUAGAUA CUGAUGAGGCCGUUAGGCCGAA IACUGCUA | 4723 |
| 555 | GUCUUAUC U AACUAUGA | 947 | UCAUAGUU CUGAUGAGGCCGUUAGGCCGAA IAUAAGAC | 4724 |
| 559 | UAUCUAAC U AUGAUGCA | 948 | UGCAUCAU CUGAUGAGGCCGUUAGGCCGAA IUUAGAUA | 4725 |
| 567 | UAUGAUGC A AAUAAAAC | 949 | GUUUUAUU CUGAUGAGGCCGUUAGGCCGAA ICAUCAUA | 4726 |
| 576 | AAUAAAAC C GGACUGAA | 950 | UUCAGUCC CUGAUGAGGCCGUUAGGCCGAA IUUUUAUU | 4727 |
| 581 | AACCGGAC U GAAGGAGC | 951 | GCUCCUUC CUGAUGAGGCCGUUAGGCCGAA IUCCGGUU | 4728 |
| 590 | GAAGGAGC U GCCCAUGA | 952 | UCAUGGGC CUGAUGAGGCCGUUAGGCCGAA ICUCCUUC | 4729 |
| 593 | GGAGCUGC C CAUGAGAA | 953 | UUCUCAUG CUGAUGAGGCCGUUAGGCCGAA ICAGCUCC | 4730 |
| 594 | GAGCUGCC C AUGAGAAA | 954 | UUUCUCAU CUGAUGAGGCCGUUAGGCCGAA IGCAGCUC | 4731 |
| 595 | AGCUGCCC A UGAGAAAU | 955 | AUUUCUCA CUGAUGAGGCCGUUAGGCCGAA IGGCAGCU | 4732 |
| 608 | AAAUUUAC A GGAAAUCC | 956 | GGAUUUCC CUGAUGAGGCCGUUAGGCCGAA IUAAAUUU | 4733 |
| 616 | AGGAAAUC C UGCAUGGC | 957 | GCCAUGCA CUGAUGAGGCCGUUAGGCCGAA IAUUUCCU | 4734 |
| 617 | GGAAAUCC U GCAUGGCG | 958 | CGCCAUGC CUGAUGAGGCCGUUAGGCCGAA IGAUUUCC | 4735 |
| 620 | AAUCCUGC A UGGCGCCG | 959 | CGGCGCCA CUGAUGAGGCCGUUAGGCCGAA ICAGGAUU | 4736 |
| 627 | CAUGGCGC C GUGCGGUU | 960 | AACCGCAC CUGAUGAGGCCGUUAGGCCGAA ICGCCAUG | 4737 |
| 637 | UGCGGUUC A GCAACAAC | 961 | GUUGUUGC CUGAUGAGGCCGUUAGGCCGAA IAACCGCA | 4738 |
| 640 | GGUUCAGC A ACAACCCU | 962 | AGGGUUGU CUGAUGAGGCCGUUAGGCCGAA ICUGAACC | 4739 |
| 643 | UCAGCAAC A ACCCUGCC | 963 | GGCAGGGU CUGAUGAGGCCGUUAGGCCGAA IUUGCUGA | 4740 |
| 646 | GCAACAAC C CUGCCCUG | 964 | CAGGGCAG CUGAUGAGGCCGUUAGGCCGAA IUUGUUGC | 4741 |
| 647 | CAACAACC C UGCCCUGU | 965 | ACAGGGCA CUGAUGAGGCCGUUAGGCCGAA IGUUGUUG | 4742 |
| 648 | AACAACCC U GCCCUGUG | 966 | CACAGGGC CUGAUGAGGCCGUUAGGCCGAA IGGUUGUU | 4743 |
| 651 | AACCCUGC C CUGUGCAA | 967 | UUGCACAG CUGAUGAGGCCGUUAGGCCGAA ICAGGGUU | 4744 |
| 652 | ACCCUGCC C UGUGCAAC | 968 | GUUGCACA CUGAUGAGGCCGUUAGGCCGAA IGCAGGGU | 4745 |
| 653 | CCCUGCCC U GUGCAACG | 969 | CGUUGCAC CUGAUGAGGCCGUUAGGCCGAA IGGCAGGG | 4746 |
| 658 | CCCUGUGC A ACGUGGAG | 970 | CUCCACGU CUGAUGAGGCCGUUAGGCCGAA ICACAGGG | 4747 |
| 670 | UGGAGAGC A UCCAGUGG | 971 | CCACUGGA CUGAUGAGGCCGUUAGGCCGAA ICUCUCCA | 4748 |
| 673 | AGAGCAUC C AGUGGCGG | 972 | CCGCCACU CUGAUGAGGCCGUUAGGCCGAA IAUGCUCU | 4749 |
| 674 | GAGCAUCC A GUGGCGGG | 973 | CCCGCCAC CUGAUGAGGCCGUUAGGCCGAA IGAUGCUC | 4750 |
| 685 | GGCGGGAC A UAGUCAGC | 974 | GCUGACUA CUGAUGAGGCCGUUAGGCCGAA IUCCCGCC | 4751 |
| 691 | ACAUAGUC A GCAGUGAC | 975 | GUCACUGC CUGAUGAGGCCGUUAGGCCGAA IACUAUGU | 4752 |
| 694 | UAGUCAGC A GUGACUUU | 976 | AAAGUCAC CUGAUGAGGCCGUUAGGCCGAA ICUGACUA | 4753 |
| 700 | GCAGUGAC U UUCUCAGC | 977 | GCUGAGAA CUGAUGAGGCCGUUAGGCCGAA IUCACUGC | 4754 |
| 704 | UGACUUUC U CAGCAACA | 978 | UGUUGCUG CUGAUGAGGCCGUUAGGCCGAA IAAAGUCA | 4755 |
| 706 | ACUUUCUC A GCAACAUG | 979 | CAUGUUGC CUGAUGAGGCCGUUAGGCCGAA IAGAAAGU | 4756 |
| 709 | UUCUCAGC A ACAUGUCG | 980 | CGACAUGU CUGAUGAGGCCGUUAGGCCGAA ICUGAGAA | 4757 |
| 712 | UCAGCAAC A UGUCGAUG | 981 | CAUCGACA CUGAUGAGGCCGUUAGGCCGAA IUUGCUGA | 4758 |
| 724 | CGAUGGAC U UCCAGAAC | 982 | GUUCUGGA CUGAUGAGGCCGUUAGGCCGAA IUCCAUCG | 4759 |
| 727 | UGGACUUC C AGAACCAC | 983 | GUGGUUCU CUGAUGAGGCCGUUAGGCCGAA IAAGUCCA | 4760 |
| 728 | GGACUUCC A GAACCACC | 984 | GGUGGUUC CUGAUGAGGCCGUUAGGCCGAA IGAAGUCC | 4761 |
| 733 | UCCAGAAC C ACCUGGGC | 985 | GCCCAGGU CUGAUGAGGCCGUUAGGCCGAA IUUCUGGA | 4762 |
| 734 | CCAGAACC A CCUGGGCA | 986 | UGCCCAGG CUGAUGAGGCCGUUAGGCCGAA IGUUCUGG | 4763 |
| 736 | AGAACCAC C UGGGCAGC | 987 | GCUGCCCA CUGAUGAGGCCGUUAGGCCGAA IUGGUUCU | 4764 |
| 737 | GAACCACC U GGGCAGCU | 988 | AGCUGCCC CUGAUGAGGCCGUUAGGCCGAA IGUGGUUC | 4765 |
| 742 | ACCUGGGC A GCUGCCAA | 989 | UUGGCAGC CUGAUGAGGCCGUUAGGCCGAA ICCCAGGU | 4766 |
| 745 | UGGGCAGC U GCCAAAAG | 990 | CUUUUGGC CUGAUGAGGCCGUUAGGCCGAA ICUGCCCA | 4767 |
| 748 | GCAGCUGC C AAAAGUGU | 991 | ACACUUUU CUGAUGAGGCCGUUAGGCCGAA ICAGCUGC | 4768 |
| 749 | CAGCUGCC A AAAGUGUG | 992 | CACACUUU CUCAUGAGGCCGUUAGGCCGAA IGCAGCUG | 4769 |
| 761 | GUGUGAUC C AAGCUGUC | 993 | GACAGCUU CUGAUGAGGCCGUUAGGCCGAA IAUCACAC | 4770 |
| 762 | UGUGAUCC A AGCUGUCC | 994 | GGACAGCU CUGAUGAGGCCGUUAGGCCGAA IGAUCACA | 4771 |
| 766 | AUCCAAGC U GUCCCAAU | 995 | AUUGGGAC CUGAUGAGGCCGUUAGGCCGAA ICUUGGAU | 4772 |
| 770 | AAGCUGUC C CAAUGGGA | 996 | UCCCAUUG CUGAUGAGGCCGUUAGGCCGAA IACAGCUU | 4773 |
| 771 | AGCUGUCC C AAUGGGAG | 997 | CUCCCAUU CUGAUGAGGCCGUUAGGCCGAA IGACAGCU | 4774 |
| 772 | GCUGUCCC A AUGGGAGC | 998 | GCUCCCAU CUGAUGAGGCCGUUAGGCCGAA IGGACAGC | 4775 |
| 781 | AUGGGAGC U GCUGGGGU | 999 | ACCCCAGC CUGAUGAGGCCGUUAGGCCGAA ICUCCCAU | 4776 |
| 784 | GGAGCUGC U GGGGUGCA | 1000 | UGCACCCC CUGAUGAGGCCGUUAGGCCGAA ICAGCUCC | 4777 |
| 792 | UGGGGUGC A GGAGAGGA | 1001 | UCCUCUCC CUGAUGAGGCCGUUAGGCCGAA ICACCCCA | 4778 |
| 805 | AGGAGAAC U GCCAGAAA | 1002 | UUUCUGGC CUGAUGAGGCCGUUAGGCCGAA IUUCUCCU | 4779 |
| 808 | AGAACUGC C AGAAACUG | 1003 | CAGUUUCU CUGAUGAGGCCGUUAGGCCGAA ICAGUUCU | 4780 |
| 809 | GAACUGCC A GAAACUGA | 1004 | UCAGUUUC CUGAUGAGGCCGUUAGGCCGAA IGCAGUUC | 4781 |
| 815 | CCAGAAAC U GACCAAAA | 1005 | UUUUGGUC CUGAUGAGGCCGUUAGGCCGAA IUUUCUGG | 4782 |
| 819 | AAACUGAC C AAAAUCAU | 1006 | AUGAUUUU CUGAUGAGGCCGUUAGGCCGAA IUCAGUUU | 4783 |
| 820 | AACUGACC A AAAUCAUC | 1007 | GAUGAUUU CUGAUGAGGCCGUUAGGCCGAA IGUCAGUU | 4784 |
| 826 | CCAAAAUC A UCUGUGCC | 1008 | GGCACAGA CUGAUGAGGCCGUUAGGCCGAA IAUUUUGG | 4785 |
| 829 | AAAUCAUC U GUGCCCAG | 1009 | CUGGGCAC CUGAUGAGGCCGUUAGGCCGAA IAUGAUUU | 4786 |
| 834 | AUCUGUGC C CAGCAGUG | 1010 | CACUGCUG CUGAUGAGGCCGUUAGGCCGAA ICACAGAU | 4787 |
| 835 | UCUGUGCC C AGCAGUGC | 1011 | GCACUGCU CUGAUGAGGCCGUUAGGCCGAA IGCACAGA | 4788 |
| 836 | CUGUGCCC A GCAGUGCU | 1012 | AGCACUGC CUGAUGAGGCCGUUAGGCCGAA IGGCACAG | 4789 |
| 839 | UGCCCAGC A GUGCUCCG | 1013 | CGGAGCAC CUGAUGAGGCCGUUAGGCCGAA ICUGGGCA | 4790 |
| 844 | AGCAGUGC U CCGGGCGC | 1014 | GCGCCCGG CUGAUGAGGCCGUUAGGCCGAA ICACUGCU | 4791 |
| 846 | CAGUGCUC C GGGCGCUG | 1015 | CAGCGCCC CUGAUGAGGCCGUUAGGCCGAA IAGCACUG | 4792 |
| 853 | CCGGGCGC U GCCGUGGC | 1016 | GCCACGGC CUGAUGAGGCCGUUAGGCCGAA ICGCCCGG | 4793 |
| 856 | GGCGCUGC C GUGGCAAG | 1017 | CUUGCCAC CUGAUGAGGCCGUUAGGCCGAA ICAGCGCC | 4794 |
| 862 | GCCGUGGC A AGUCCCCC | 1018 | GGGGGACU CUGAUGAGGCCGUUAGGCCGAA ICCACGGC | 4795 |
| 867 | GGCAAGUC C CCCAGUGA | 1019 | UCACUGGG CUGAUGAGGCCGUUAGGCCGAA IACUUGCC | 4796 |
| 868 | GCAAGUCC C CCAGUGAC | 1020 | GUCACUGG CUGAUGAGGCCGUUAGGCCGAA IGACUUGC | 4797 |
| 869 | CAAGUCCC C CAGUGACU | 1021 | AGUCACUG CUGAUGAGGCCGUUAGGCCGAA IGGACUUG | 4798 |
| 870 | AAGUCCCC C AGUGACUG | 1022 | CAGUCACU CUGAUGAGGCCGUUAGGCCGAA IGGGACUU | 4799 |
| 871 | AGUCCCCC A GUGACUGC | 1023 | GCAGUCAC CUGAUGAGGCCGUUAGGCCGAA IGGGGACU | 4800 |
| 877 | CCAGUGAC U GCUGCCAC | 1024 | GUGGCAGC CUGAUGAGGCCGUUAGGCCGAA IUCACUGG | 4801 |
| 880 | GUGACUGC U GCCACAAC | 1025 | GUUGUGGC CUGAUGAGGCCGUUAGGCCGAA ICAGUCAC | 4802 |
| 883 | ACUGCUGC C ACAACCAG | 1026 | CUGGUUGU CUGAUGAGGCCGUUAGGCCGAA ICAGCAGU | 4803 |
| 884 | CUGCUGCC A CAACCAGU | 1027 | ACUGGUUG CUGAUGAGGCCGUUAGGCCGAA IGCAGCAG | 4804 |
| 886 | GCUGCCAC A ACCAGUGU | 1028 | ACACUGGU CUGAUGAGGCCGUUAGGCCGAA IUGGCAGC | 4805 |
| 889 | GCCACAAC C AGUGUGCU | 1029 | AGCACACU CUGAUGAGGCCGUUAGGCCGAA IUUGUGGC | 4806 |
| 890 | CCACAACC A GUGUGCUG | 1030 | CAGCACAC CUGAUGAGGCCGUUAGGCCGAA IGUUGUGG | 4807 |
| 897 | CAGUGUGC U GCAGGCUG | 1031 | CAGCCUGC CUGAUGAGGCCGUUAGGCCGAA ICACACUG | 4808 |
| 900 | UGUGCUGC A GGCUGCAC | 1032 | GUGCAGCC CUGAUGAGGCCGUUAGGCCGAA ICAGCACA | 4809 |
| 904 | CUGCAGGC U GCACAGGC | 1033 | GCCUGUGC CUGAUGAGGCCGUUAGGCCGAA ICCUGCAG | 4810 |
| 907 | CAGGCUGC A CAGGCCCC | 1034 | GGGGCCUG CUGAUGAGGCCGUUAGGCCGAA ICAGCCUG | 4811 |
| 909 | GGCUGCAC A GGCCCCCG | 1035 | CGGGGGCC CUGAUGAGGCCGUUAGGCCGAA IUGCAGCC | 4812 |
| 913 | GCACAGGC C CCCGGGAG | 1036 | CUCCCGGG CUGAUGAGGCCGUUAGGCCGAA ICCUGUGC | 4813 |
| 914 | CACAGGCC C CCGGGAGA | 1037 | UCUCCCGG CUGAUGAGGCCGUUAGGCCGAA IGCCUGUG | 4814 |
| 915 | ACAGGCCC C CGGGAGAG | 1038 | CUCUCCCG CUGAUGAGGCCGUUAGGCCGAA IGGCCUGU | 4815 |
| 916 | CAGGCCCC C GGGAGAGC | 1039 | GCUCUCCC CUGAUGAGGCCGUUAGGCCGAA IGGGCCUG | 4816 |
| 928 | AGAGCGAC U GCCUGGUC | 1040 | GACCAGGC CUGAUGAGGCCGUUAGGCCGAA IUCGCUCU | 4817 |
| 931 | GCGACUGC C UGGUCUGC | 1041 | GCAGACCA CUGAUGAGGCCGUUAGGCCGAA ICAGUCGC | 4818 |
| 932 | CGACUGCC U GGUCUGCC | 1042 | GGCAGACC CUGAUGAGGCCGUUAGGCCGAA IGCAGUCG | 4819 |
| 937 | GCCUGGUC U GCCGCAAA | 1043 | UUUGCGGC CUGAUGAGGCCGUUAGGCCGAA IACCAGGC | 4820 |
| 940 | UGGUCUGC C GCAAAUUC | 1044 | GAAUUUGC CUGAUGAGGCCGUUAGGCCGAA ICAGACCA | 4821 |
| 943 | UCUGCCGC A AAUUCCGA | 1045 | UCGGAAUU CUGAUGAGGCCGUUAGGCCGAA ICGGCAGA | 4822 |
| 949 | GCAAAUUC C GAGACGAA | 1046 | UUCGUCUC CUGAUGAGGCCGUUAGGCCGAA IAAUUUGC | 4823 |
| 960 | GACGAAGC C ACGUGCAA | 1047 | UUGCACGU CUGAUGAGGCCGUUAGGCCGAA ICUUCGUC | 4824 |
| 961 | ACGAAGCC A CGUGCAAG | 1048 | CUUGCACG CUGAUGAGGCCGUUAGGCCGAA IGCUUCGU | 4825 |
| 967 | CCACGUGC A AGGACACC | 1049 | GGUGUCCU CUGAUGAGGCCGUUAGGCCGAA ICACGUGG | 4826 |
| 973 | GCAAGGAC A CCUGCCCC | 1050 | GGGGCAGG CUGAUGAGGCCGUUAGGCCGAA IUCCUUGC | 4827 |
| 975 | AAGGACAC C UGCCCCCC | 1051 | GGGGGGCA CUGAUGAGGCCGUUAGGCCGAA IUGUCCUU | 4828 |
| 976 | AGGACACC U GCCCCCCA | 1052 | UGGGGGGC CUGAUGAGGCCGUUAGGCCGAA IGUGUCCU | 4829 |
| 979 | ACACCUGC C CCCCACUC | 1053 | GAGUGGGG CUGAUGAGGCCGUUAGGCCGAA ICAGGUGU | 4830 |
| 980 | CACCUGCC C CCCACUCA | 1054 | UGAGUGGG CUGAUGAGGCCGUUAGGCCGAA IGCAGGUG | 4831 |
| 981 | ACCUGCCC C CCACUCAU | 1055 | AUGAGUGG CUGAUGAGGCCGUUAGGCCGAA IGGCAGGU | 4832 |
| 982 | CCUGCCCC C CACUCAUG | 1056 | CAUGAGUG CUGAUGAGGCCGUUAGGCCGAA IGGGCAGG | 4833 |
| 983 | CUGCCCCC C ACUCAUGC | 1057 | GCAUGAGU CUGAUGAGGCCGUUAGGCCGAA IGGGGCAG | 4834 |
| 984 | UGCCCCCC A CUCAUGCU | 1058 | AGCAUGAG CUGAUGAGGCCGUUAGGCCGAA IGGGGGCA | 4835 |
| 986 | CCCCCCAC U CAUGCUCU | 1059 | AGAGCAUG CUGAUGAGGCCGUUAGGCCGAA IUGGGGGG | 4836 |
| 988 | CCCCACUC A UGCUCUAC | 1060 | GUAGAGCA CUGAUGAGGCCGUUAGGCCGAA IAGUGGGG | 4837 |
| 992 | ACUCAUGC U CUACAACC | 1061 | GGUUGUAG CUGAUGAGGCCGUUAGGCCGAA ICAUGAGU | 4838 |
| 994 | UCAUGCUC U ACAACCCC | 1062 | GGGGUUGU CUGAUGAGGCCGUUAGGCCGAA IAGCAUGA | 4839 |
| 997 | UGCUCUAC A ACCCCACC | 1063 | GGUGGGGU CUGAUGAGGCCGUUAGGCCGAA IUAGAGCA | 4840 |
| 1000 | UCUACAAC C CCACCACG | 1064 | CGUGGUGG CUGAUGAGGCCGUUAGGCCGAA IUUGUAGA | 4841 |
| 1001 | CUACAACC C CACCACGU | 1065 | ACGUGGUG CUGAUGAGGCCGUUAGGCCGAA IGUUGUAG | 4842 |
| 1002 | UACAACCC C ACCACGUA | 1066 | UACGUGGU CUGAUGAGGCCGUUAGGCCGAA IGGUUGUA | 4843 |
| 1003 | ACAACCCC A CCACGUAC | 1067 | GUACGUGG CUGAUGAGGCCGUUAGGCCGAA IGGGUUGU | 4844 |
| 1005 | AACCCCAC C ACGUACCA | 1068 | UGGUACGU CUGAUGAGGCCGUUAGGCCGAA IUGGGGUU | 4845 |
| 1006 | ACCCCACC A CGUACCAG | 1069 | CUGGUACG CUGAUGAGGCCGUUAGGCCGAA IGUGGGGU | 4846 |
| 1012 | CCACGUAC C AGAUGGAU | 1070 | AUCCAUCU CUGAUGAGGCCGUUAGGCCGAA IUACGUGG | 4847 |
| 1013 | CACGUACC A GAUGGAUG | 1071 | CAUCCAUC CUGAUGAGGCCGUUAGGCCGAA IGUACGUG | 4848 |
| 1027 | AUGUGAAC C CCGAGGGC | 1072 | GCCCUCGG CUGAUGAGGCCGUUAGGCCGAA IUUCACAU | 4849 |
| 1028 | UGUGAACC C CGAGGGCA | 1073 | UGCCCUCG CUGAUGAGGCCGUUAGGCCGAA IGUUCACA | 4850 |
| 1029 | GUGAACCC C GAGGGCAA | 1074 | UUGCCCUC CUGAUGAGGCCGUUAGGCCGAA IGGUUCAC | 4851 |
| 1036 | CCGAGGGC A AAUACAGC | 1075 | GCUGUAUU CUGAUGAGGCCGUUAGGCCGAA ICCCUCGG | 4852 |
| 1042 | GCAAAUAC A GCUUUGGU | 1076 | ACCAAAGC CUGAUGAGGCCGUUAGGCCGAA IUAUUUGC | 4853 |
| 1045 | AAUACAGC U UUGGUGCC | 1077 | GGCACCAA CUGAUGAGGCCGUUAGGCCGAA ICUGUAUU | 4854 |
| 1053 | UUUGGUGC C ACCUGCGU | 1078 | ACGCAGGU CUGAUGAGGCCGUUAGGCCGAA ICACCAAA | 4855 |
| 1054 | UUGGUGCC A CCUGCGUG | 1079 | CACGCAGG CUGAUGAGGCCGUUAGGCCGAA IGCACCAA | 4856 |
| 1056 | GGUGCCAC C UGCGUGAA | 1080 | UUCACGCA CUGAUGAGGCCGUUAGGCCGAA IUGGCACC | 4857 |
| 1057 | GUGCCACC U GCGUGAAG | 1081 | CUUCACGC CUGAUGAGGCCGUUAGGCCGAA IGUGGCAC | 4858 |
| 1073 | GAAGUGUC C CCGUAAUU | 1082 | AAUUACGG CUGAUGAGGCCGUUAGGCCGAA IACACUUC | 4859 |
| 1074 | AAGUGUCC C CGUAAUUA | 1083 | UAAUUACG CUGAUGAGGCCGUUAGGCCGAA IGACACUU | 4860 |
| 1075 | AGUGUCCC C GUAAUUAU | 1084 | AUAAUUAC CUGAUGAGGCCGUUAGGCCGAA IGGACACU | 4861 |
| 1092 | GUGGUGAC A GAUCACGG | 1085 | CCGUGAUC CUGAUGAGGCCGUUAGGCCGAA IUCACCAC | 4862 |
| 1097 | GACAGAUC A CGGCUCGU | 1086 | ACGAGCCG CUGAUGAGGCCGUUAGGCCGAA IAUCUGUC | 4863 |
| 1102 | AUCACGGC U CGUGCGUC | 1087 | GACGCACG CUGAUGAGGCCGUUAGGCCGAA ICCGUGAU | 4864 |
| 1111 | CGUGCGUC C GAGCCUGU | 1088 | ACAGGCUC CUGAUGAGGCCGUUAGGCCGAA IACGCACG | 4865 |
| 1116 | GUCCGAGC C UGUGGGGC | 1089 | GCCCCACA CUGAUGAGGCCGUUAGGCCGAA ICUCGGAC | 4866 |
| 1117 | UCCGAGCC U GUGGGGCC | 1090 | GGCCCCAC CUGAUGAGGCCGUUAGGCCGAA IGCUCGGA | 4867 |
| 1125 | UGUGGGGC C GACAGCUA | 1091 | UAGCUGUC CUGAUGAGGCCGUUAGGCCGAA ICCCCACA | 4868 |
| 1129 | GGGCCGAC A GCUAUGAG | 1092 | CUCAUAGC CUGAUGAGGCCGUUAGGCCGAA IUCGGCCC | 4869 |
| 1132 | CCGACAGC U AUGAGAUG | 1093 | CAUCUCAU CUGAUGAGGCCGUUAGGCCGAA ICUGUCGG | 4870 |
| 1156 | ACGGCGUC C GCAAGUGU | 1094 | ACACUUGC CUGAUGAGGCCGUUAGGCCGAA IACGCCGU | 4871 |
| 1159 | GCGUCCGC A AGUGUAAG | 1095 | CUUACACU CUGAUGAGGCCGUUAGGCCGAA ICGGACGC | 4872 |
| 1181 | CGAAGGGC C UUGCCGCA | 1096 | UGCGGCAA CUGAUGAGGCCGUUAGGCCGAA ICCCUUCG | 4873 |
| 1182 | GAAGGGCC U UGCCGCAA | 1097 | UUGCGGCA CUGAUGAGGCCGUUAGGCCGAA IGCCCUUC | 4874 |
| 1186 | GGCCUUGC C GCAAAGUG | 1098 | CACUUUGC CUGAUGAGGCCGUUAGGCCGAA ICAAGGCC | 4875 |
| 1189 | CUUGCCGC A AAGUGUGU | 1099 | ACACACUU CUGAUGAGGCCGUUAGGCCGAA ICGGCAAG | 4876 |
| 1228 | UUAAAGAC U CACUCUCC | 1100 | GGAGAGUG CUGAUGAGGCCGUUAGGCCGAA IUCUUUAA | 4877 |
| 1230 | AAAGACUC A CUCUCCAU | 1101 | AUGGAGAG CUGAUGAGGCCGUUAGGCCGAA IAGUCUUU | 4878 |
| 1232 | AGACUCAC U CUCCAUAA | 1102 | UUAUGGAG CUGAUGAGGCCGUUAGGCCGAA IUGAGUCU | 4879 |
| 1234 | ACUCACUC U CCAUAAAU | 1103 | AUUUAUGG CUGAUGAGGCCGUUAGGCCGAA IAGUGAGU | 4880 |
| 1236 | UCACUCUC C AUAAAUGC | 1104 | GCAUUUAU CUGAUGAGGCCGUUAGGCCGAA IAGAGUGA | 4881 |
| 1237 | CACUCUCC A UAAAUGCU | 1105 | AGCAUUUA CUGAUGAGGCCGUUAGGCCGAA IGAGAGUG | 4882 |
| 1245 | AUAAAUGC U ACGAAUAU | 1106 | AUAUUCGU CUGAUGAGGCCGUUAGGCCGAA ICAUUUAU | 4883 |
| 1259 | UAUUAAAC A CUUCAAAA | 1107 | UUUUGAAG CUGAUGAGGCCGUUAGGCCGAA IUUUAAUA | 4884 |
| 1261 | UUAAACAC U UCAAAAAC | 1108 | GUUUUUGA CUGAUGAGGCCGUUAGGCCGAA IUGUUUAA | 4885 |
| 1264 | AACACUUC A AAAACUGC | 1109 | GCAGUUUU CUGAUGAGGCCGUUAGGCCGAA IAAGUGUU | 4886 |
| 1270 | UCAAAAAC U GCACCUCC | 1110 | GGAGGUGC CUGAUGAGGCCGUUAGGCCGAA IUUUUUGA | 4887 |
| 1273 | AAAACUGC A CCUCCAUC | 1111 | GAUGGAGG CUGAUGAGGCCGUUAGGCCGAA ICAGUUUU | 4888 |
| 1275 | AACUGCAC C UCCAUCAG | 1112 | CUGAUGGA CUGAUGAGGCCGUUAGGCCGAA IUGCAGUU | 4889 |
| 1276 | ACUGCACC U CCAUCAGU | 1113 | ACUGAUGG CUGAUGAGGCCGUUAGGCCGAA IGUGCAGU | 4890 |
| 1278 | UGCACCUC C AUCAGUGG | 1114 | CCACUGAU CUGAUGAGGCCGUUAGGCCGAA IAGGUGCA | 4891 |
| 1279 | GCACCUCC A UCAGUGGC | 1115 | GCCACUGA CUGAUGAGGCCGUUAGGCCGAA IGAGGUGC | 4892 |
| 1282 | CCUCCAUC A GUGGCGAU | 1116 | AUCGCCAC CUGAUGAGGCCGUUAGGCCGAA IAUGGAGG | 4893 |
| 1292 | UGGCGAUC U CCACAUCC | 1117 | GGAUGUGG CUGAUGAGGCCGUUAGGCCGAA IAUCGCCA | 4894 |
| 1294 | GCGAUCUC C ACAUCCUG | 1118 | CAGGAUGU CUGAUGAGGCCGUUAGGCCGAA IAGAUCGC | 4895 |
| 1295 | CGAUCUCC A CAUCCUGC | 1119 | GCAGGAUG CUGAUGAGGCCGUUAGGCCGAA IGAGAUCG | 4896 |
| 1297 | AUCUCCAC A UCCUGCCG | 1120 | CGGCAGGA CUGAUGAGGCCGUUAGGCCGAA IUGGAGAU | 4897 |
| 1300 | UCCACAUC C UGCCGGUG | 1121 | CACCGGCA CUGAUGAGGCCGUUAGGCCGAA IAUGUGGA | 4898 |
| 1301 | CCACAUCC U GCCGGUGG | 1122 | CCACCGGC CUGAUGAGGCCGUUAGGCCGAA IGAUGUGG | 4899 |
| 1304 | CAUCCUGC C GGUGGCAU | 1123 | AUGCCACC CUGAUGAGGCCGUUAGGCCGAA ICAGGAUG | 4900 |
| 1311 | CCGGUGGC A UUUAGGGG | 1124 | CCCCUAAA CUGAUGAGGCCGUUAGGCCGAA ICCACCGG | 4901 |
| 1324 | GGGGUGAC U CCUUCACA | 1125 | UGUGAAGG CUGAUGAGGCCGUUAGGCCGAA IUCACCCC | 4902 |
| 1326 | GGUGACUC C UUCACACA | 1126 | UGUGUGAA CUGAUGAGGCCGUUAGGCCGAA IAGUCACC | 4903 |
| 1327 | GUGACUCC U UCACACAU | 1127 | AUGUGUGA CUGAUGAGGCCGUUAGGCCGAA IGAGUCAC | 4904 |
| 1330 | ACUCCUUC A CACAUACU | 1128 | AGUAUGUG CUGAUGAGGCCGUUAGGCCGAA IAAGGAGU | 4905 |
| 1332 | UCCUUCAC A CAUACUCC | 1129 | GGAGUAUG CUGAUGAGGCCGUUAGGCCGAA IUGAAGGA | 4906 |
| 1334 | CUUCACAC A UACUCCUC | 1130 | GAGGAGUA CUGAUGAGGCCGUUAGGCCGAA IUGUGAAG | 4907 |
| 1338 | ACACAUAC U CCUCCUCU | 1131 | AGAGGAGG CUGAUGAGGCCGUUAGGCCGAA IUAUGUGU | 4908 |
| 1340 | ACAUACUC C UCCUCUGG | 1132 | CCAGAGGA CUGAUGAGGCCGUUAGGCCGAA IAGUAUGU | 4909 |
| 1341 | CAUACUCC U CCUCUGGA | 1133 | UCCAGAGG CUGAUGAGGCCGUUAGGCCGAA IGAGUAUG | 4910 |
| 1343 | UACUCCUC C UCUGGAUC | 1134 | GAUCCAGA CUGAUGAGGCCGUUAGGCCGAA IAGGAGUA | 4911 |
| 1344 | ACUCCUCC U CUGGAUCC | 1135 | GGAUCCAG CUGAUGAGGCCGUUAGGCCGAA IGAGGAGU | 4912 |
| 1346 | UCCUCCUC U GGAUCCAC | 1136 | GUGGAUCC CUGAUGAGGCCGUUAGGCCGAA IAGGAGGA | 4913 |
| 1352 | UCUGGAUC C ACAGGAAC | 1137 | GUUCCUGU CUGAUGAGGCCGUUAGGCCGAA IAUCCAGA | 4914 |
| 1353 | CUGGAUCC A CAGGAACU | 1138 | AGUUCCUG CUGAUGAGGCCGUUAGGCCGAA IGAUCCAG | 4915 |
| 1355 | GGAUCCAC A GGAACUGG | 1139 | CCAGUUCC CUGAUGAGGCCGUUAGGCCGAA IUGGAUCC | 4916 |
| 1361 | ACAGGAAC U GGAUAUUC | 1140 | GAAUAUCC CUGAUGAGGCCGUUAGGCCGAA IUUCCUGU | 4917 |
| 1370 | GGAUAUUC U GAAAACCG | 1141 | CGGUUUUC CUGAUGAGGCCGUUAGGCCGAA IAAUAUCC | 4918 |
| 1377 | CUGAAAAC C GUAAAGGA | 1142 | UCCUUUAC CUGAUGAGGCCGUUAGGCCGAA IUUUUCAG | 4919 |
| 1390 | AGGAAAUC A CAGGGUUU | 1143 | AAACCCUG CUGAUGAGGCCGUUAGGCCGAA IAUUUCCU | 4920 |
| 1392 | GAAAUCAC A GGGUUUUU | 1144 | AAAAACCC CUGAUGAGGCCGUUAGGCCGAA IUGAUUUC | 4921 |
| 1403 | GUUUUUGC U GAUUCAGG | 1145 | CCUGAAUC CUGAUGAGGCCGUUAGGCCGAA ICAAAAAC | 4922 |
| 1409 | GCUGAUUC A GGCUUGGC | 1146 | GCCAAGCC CUGAUGAGGCCGUUAGGCCGAA IAAUCAGC | 4923 |
| 1413 | AUUCAGGC U UGGCCUGA | 1147 | UCAGGCCA CUGAUGAGGCCGUUAGGCCGAA ICCUGAAU | 4924 |
| 1418 | GGCUUGGC C UGAAAACA | 1148 | UGUUUUCA CUGAUGAGGCCGUUAGGCCGAA ICCAAGCC | 4925 |
| 1419 | GCUUGGCC U GAAAACAG | 1149 | CUGUUUUC CUGAUGAGGCCGUUAGGCCGAA IGCCAAGC | 4926 |
| 1426 | CUGAAAAC A GGACGGAC | 1150 | GUCCGUCC CUGAUGAGGCCGUUAGGCCGAA IUUUUCAG | 4927 |
| 1435 | GGACGGAC C UCCAUGCC | 1151 | GGCAUGGA CUGAUGAGGCCGUUAGGCCGAA IUCCGUCC | 4928 |
| 1436 | GACGGACC U CCAUGCCU | 1152 | AGGCAUGG CUGAUGAGGCCGUUAGGCCGAA IGUCCGUC | 4929 |
| 1438 | CGGACCUC C AUGCCUUU | 1153 | AAAGGCAU CUGAUGAGGCCGUUAGGCCGAA IAGGUCCG | 4930 |
| 1439 | GGACCUCC A UGCCUUUG | 1154 | CAAAGGCA CUGAUGAGGCCGUUAGGCCGAA IGAGGUCC | 4931 |
| 1443 | CUCCAUGC C UUUGAGAA | 1155 | UUCUCAAA CUGAUGAGGCCGUUAGGCCGAA ICAUGGAG | 4932 |
| 1444 | UCCAUGCC U UUGAGAAC | 1156 | GUUCUCAA CUGAUGAGGCCGUUAGGCCGAA IGCAUGGA | 4933 |
| 1453 | UUGAGAAC C UAGAAAUC | 1157 | GAUUUCUA CUGAUGAGGCCGUUAGGCCGAA IUUCUCAA | 4934 |
| 1454 | UGAGAACC U AGAAAUCA | 1158 | UGAUUUCU CUGAUGAGGCCGUUAGGCCGAA IGUUCUCA | 4935 |
| 1462 | UAGAAAUC A UACGCGGC | 1159 | GCCGCGUA CUGAUGAGGCCGUUAGGCCGAA IAUUUCUA | 4936 |
| 1471 | UACGCGGC A GGACCAAG | 1160 | CUUGGUCC CUGAUGAGGCCGUUAGGCCGAA ICCGCGUA | 4937 |
| 1476 | GGCAGGAC C AAGCAACA | 1161 | UGUUGCUU CUGAUGAGGCCGUUAGGCCGAA IUCCUGCC | 4938 |
| 1477 | GCAGGACC A AGCAACAU | 1162 | AUGUUGCU CUGAUGAGGCCGUUAGGCCGAA IGUCCUGC | 4939 |
| 1481 | GACCAAGC A ACAUGGUC | 1163 | GACCAUGU CUGAUGAGGCCGUUAGGCCGAA ICUUGGUC | 4940 |
| 1484 | CAAGCAAC A UGGUCAGU | 1164 | ACUGACCA CUGAUGAGGCCGUUAGGCCGAA IUUGCUUG | 4941 |
| 1490 | ACAUGGUC A GUUUUCUC | 1165 | GAGAAAAC CUGAUGAGGCCGUUAGGCCGAA IACCAUGU | 4942 |
| 1497 | CAGUUUUC U CUUGCAGU | 1166 | ACUGCAAG CUGAUGAGGCCGUUAGGCCGAA IAAAACUG | 4943 |
| 1499 | GUUUUCUC U UGCAGUCG | 1167 | CGACUGCA CUGAUGAGGCCGUUAGGCCGAA IAGAAAAC | 4944 |
| 1503 | UCUCUUGC A GUCGUCAG | 1168 | CUGACGAC CUGAUGAGGCCGUUAGGCCGAA ICAAGAGA | 4945 |
| 1510 | CAGUCGUC A GCCUGAAC | 1169 | GUUCAGGC CUGAUGAGGCCGUUAGGCCGAA IACGACUG | 4946 |
| 1513 | UCGUCAGC C UGAACAUA | 1170 | UAUGUUCA CUGAUGAGGCCGUUAGGCCGAA ICUGACGA | 4947 |
| 1514 | CGUCAGCC U GAACAUAA | 1171 | UUAUGUUC CUGAUGAGGCCGUUAGGCCGAA IGCUGACG | 4948 |
| 1519 | GCCUGAAC A UAACAUCC | 1172 | GGAUGUUA CUGAUGAGGCCGUUAGGCCGAA IUUCAGGC | 4949 |
| 1524 | AACAUAAC A UCCUUGGG | 1173 | CCCAAGGA CUGAUGAGGCCGUUAGGCCGAA IUUAUGUU | 4950 |
| 1527 | AUAACAUC C UUGGGAUU | 1174 | AAUCCCAA CUGAUGAGGCCGUUAGGCCGAA IAUGUUAU | 4951 |
| 1528 | UAACAUCC U UGGGAUUA | 1175 | UAAUCCCA CUGAUGAGGCCGUUAGGCCGAA IGAUGUUA | 4952 |
| 1540 | GAUUACGC U CCCUCAAG | 1176 | CUUGAGGG CUGAUGAGGCCGUUAGGCCGAA ICGUAAUC | 4953 |
| 1542 | UUACGCUC C CUCAAGGA | 1177 | UCCUUGAG CUGAUGAGGCCGUUAGGCCGAA IAGCGUAA | 4954 |
| 1543 | UACGCUCC C UCAAGGAG | 1178 | CUCCUUGA CUGAUGAGGCCGUUAGGCCGAA IGAGCGUA | 4955 |
| 1544 | ACGCUCCC U CAAGGAGA | 1179 | UCUCCUUG CUGAUGAGGCCGUUAGGCCGAA IGGAGCGU | 4956 |
| 1546 | GCUCCCUC A AGGAGAUA | 1180 | UAUCUCCU CUGAUGAGGCCGUUAGGCCGAA IAGGGAGC | 4957 |
| 1578 | AUAAUUUC A GGAAACAA | 1181 | UUGUUUCC CUGAUGAGGCCGUUAGGCCGAA IAAAUUAU | 4958 |
| 1585 | CAGGAAAC A AAAAUUUG | 1182 | CAAAUUUU CUGAUGAGGCCGUUAGGCCGAA IUUUCCUG | 4959 |
| 1597 | AUUUGUGC U AUGCAAAU | 1183 | AUUUGCAU CUGAUGAGGCCGUUAGGCCGAA ICACAAAU | 4960 |
| 1602 | UGCUAUGC A AAUACAAU | 1184 | AUUGUAUU CUGAUGAGGCCGUUAGGCCGAA ICAUAGCA | 4961 |
| 1608 | GCAAAUAC A AUAAACUG | 1185 | CAGUUUAU CUGAUGAGGCCGUUAGGCCGAA IUAUUUGC | 4962 |
| 1615 | CAAUAAAC U GGAAAAAA | 1186 | UUUUUUCC CUGAUGAGGCCGUUAGGCCGAA IUUUAUUG | 4963 |
| 1625 | GAAAAAAC U GUUUGGGA | 1187 | UCCCAAAC CUGAUGAGGCCGUUAGGCCGAA IUUUUUUC | 4964 |
| 1635 | UUUGGGAC C UCCGGUCA | 1188 | UGACCGGA CUGAUGAGGCCGUUAGGCCGAA IUCCCAAA | 4965 |
| 1636 | UUGGGACC U CCGGUCAG | 1189 | CUGACCGG CUGAUGAGGCCGUUAGGCCGAA IGUCCCAA | 4966 |
| 1638 | GGGACCUC C GGUCAGAA | 1190 | UUCUGACC CUGAUGAGGCCGUUAGGCCGAA IAGGUCCC | 4967 |
| 1643 | CUCCGGUC A GAAAACCA | 1191 | UGGUUUUC CUGAUGAGGCCGUUAGGCCGAA IACCGGAG | 4968 |
| 1650 | CAGAAAAC C AAAAUUAU | 1192 | AUAAUUUU CUGAUGAGGCCGUUAGGCCGAA IUUUUCUG | 4969 |
| 1651 | AGAAAACC A AAAUUAUA | 1193 | UAUAAUUU CUGAUGAGGCCGUUAGGCCGAA IGUUUUCU | 4970 |
| 1663 | UUAUAAGC A ACAGAGGU | 1194 | ACCUCUGU CUGAUGAGGCCGUUAGGCCGAA ICUUAUAA | 4971 |
| 1666 | UAAGCAAC A GAGGUGAA | 1195 | UUCACCUC CUGAUGAGGCCGUUAGGCCGAA IUUGCUUA | 4972 |
| 1678 | GUGAAAAC A GCUGCAAG | 1196 | CUUGCAGC CUGAUGAGGCCGUUAGGCCGAA IUUUUCAC | 4973 |
| 1681 | AAAACAGC U GCAAGGCC | 1197 | GGCCUUGC CUGAUGAGGCCGUUAGGCCGAA ICUGUUUU | 4974 |
| 1684 | ACAGCUGC A AGGCCACA | 1198 | UGUGGCCU CUGAUGAGGCCGUUAGGCCGAA ICAGCUGU | 4975 |
| 1689 | UGCAAGGC C ACAGGCCA | 1199 | UGGCCUGU CUGAUGAGGCCGUUAGGCCGAA ICCUUGCA | 4976 |
| 1690 | GCAAGGCC A CAGGCCAG | 1200 | CUGGCCUG CUGAUGAGGCCGUUAGGCCGAA IGCCUUGC | 4977 |
| 1692 | AAGGCCAC A GGCCAGGU | 1201 | ACCUGGCC CUGAUGAGGCCGUUAGGCCGAA IUGGCCUU | 4978 |
| 1696 | CCACAGGC C AGGUCUGC | 1202 | GCAGACCU CUGAUGAGGCCGUUAGGCCGAA ICCUGUGG | 4979 |
| 1697 | CACAGGCC A GGUCUGCC | 1203 | GGCAGACC CUGAUGAGGCCGUUAGGCCGAA IGCCUGUG | 4980 |
| 1702 | GCCAGGUC U GCCAUGCC | 1204 | GGCAUGGC CUGAUGAGGCCGUUAGGCCGAA IACCUGGC | 4981 |
| 1705 | AGGUCUGC C AUGCCUUG | 1205 | CAAGGCAU CUGAUGAGGCCGUUAGGCCGAA ICAGACCU | 4982 |
| 1706 | GGUCUGCC A UGCCUUGU | 1206 | ACAAGGCA CUGAUGAGGCCGUUAGGCCGAA IGCAGACC | 4983 |
| 1710 | UGCCAUGC C UUGUGCUC | 1207 | GAGCACAA CUGAUGAGGCCGUUAGGCCGAA ICAUGGCA | 4984 |
| 1711 | GCCAUGCC U UGUGCUCC | 1208 | GGAGCACA CUGAUGAGGCCGUUAGGCCGAA IGCAUGGC | 4985 |
| 1717 | CCUUGUGC U CCCCCGAG | 1209 | CUCGGGGG CUGAUGAGGCCGUUAGGCCGAA ICACAAGG | 4986 |
| 1719 | UUGUGCUC C CCCGAGGG | 1210 | CCCUCGGG CUGAUGAGGCCGUUAGGCCGAA IAGCACAA | 4987 |
| 1720 | UGUGCUCC C CCGAGGGC | 1211 | GCCCUCGG CUGAUGAGGCCGUUAGGCCGAA IGAGCACA | 4988 |
| 1721 | GUGCUCCC C CGAGGGCU | 1212 | AGCCCUCG CUGAUGAGGCCGUUAGGCCGAA IGGAGCAC | 4989 |
| 1722 | UGCUCCCC C GAGGGCUG | 1213 | CAGCCCUC CUGAUGAGGCCGUUAGGCCGAA IGGGAGCA | 4990 |
| 1729 | CCGAGGGC U GCUGGGGC | 1214 | GCCCCAGC CUGAUGAGGCCGUUAGGCCGAA ICCCUCGG | 4991 |
| 1732 | AGGGCUGC U GGGGCCCG | 1215 | CGGGCCCC CUGAUGAGGCCGUUAGGCCGAA ICAGCCCU | 4992 |
| 1738 | GCUGGGGC C CGGAGCCC | 1216 | GGGCUCCG CUGAUGAGGCCGUUAGGCCGAA ICCCCAGC | 4993 |
| 1739 | CUGGGGCC C GGAGCCCA | 1217 | UGGGCUCC CUGAUGAGGCCGUUAGGCCGAA IGCCCCAG | 4994 |
| 1745 | CCCGGAGC C CAGGGACU | 1218 | AGUCCCUG CUGAUGAGGCCGUUAGGCCGAA ICUCCGGG | 4995 |
| 1746 | CCGGAGCC C AGGGACUG | 1219 | CAGUCCCU CUGAUGAGGCCGUUAGGCCGAA IGCUCCGG | 4996 |
| 1747 | CGGAGCCC A GGGACUGC | 1220 | GCAGUCCC CUGAUGAGGCCGUUAGGCCGAA IGGCUCCG | 4997 |
| 1753 | CCAGGGAC U GCGUCUCU | 1221 | AGAGACGC CUGAUGAGGCCGUUAGGCCGAA IUCCCUGG | 4998 |
| 1759 | ACUGCGUC U CUUGCCGG | 1222 | CCGGCAAG CUGAUGAGGCCGUUAGGCCGAA IACGCAGU | 4999 |
| 1761 | UGCGUCUC U UGCCGGAA | 1223 | UUCCGGCA CUGAUGAGGCCGUUAGGCCGAA IAGACGCA | 5000 |
| 1765 | UCUCUUGC C GGAAUGUC | 1224 | GACAUUCC CUGAUGAGGCCGUUAGGCCGAA ICAAGAGA | 5001 |
| 1774 | GGAAUGUC A GCCGAGGC | 1225 | GCCUCGGC CUGAUGAGGCCGUUAGGCCGAA IACAUUCC | 5002 |
| 1777 | AUGUCAGC C GAGGCAGG | 1226 | CCUGCCUC CUGAUGAGGCCGUUAGGCCGAA ICUGACAU | 5003 |
| 1783 | GCCGAGGC A GGGAAUGC | 1227 | GCAUUCCC CUGAUGAGGCCGUUAGGCCGAA ICCUCGGC | 5004 |
| 1798 | GCGUGGAC A AGUGCAAG | 1228 | CUUGCACU CUGAUGAGGCCGUUAGGCCGAA IUCCACGC | 5005 |
| 1804 | ACAAGUGC A AGCUUCUG | 1229 | CAGAAGCU CUGAUGAGGCCGUUAGGCCGAA ICACUUGU | 5006 |
| 1808 | GUGCAAGC U UCUGGAGG | 1230 | CCUCCAGA CUGAUGAGGCCGUUAGGCCGAA ICUUGCAC | 5007 |
| 1811 | CAAGCUUC U GGAGGGUG | 1231 | CACCCUCC CUGAUGAGGCCGUUAGGCCGAA IAAGCUUG | 5008 |
| 1823 | GGGUGAGC C AAGGGAGU | 1232 | ACUCCCUU CUGAUGAGGCCGUUAGGCCGAA ICUCACCC | 5009 |
| 1824 | GGUGAGCC A AGGGAGUU | 1233 | AACUCCCU CUGAUGAGGCCGUUAGGCCGAA IGCUCACC | 5010 |
| 1843 | UGGAGAAC U CUGAGUGC | 1234 | GCACUCAG CUGAUGAGGCCGUUAGGCCGAA IUUCUCCA | 5011 |
| 1845 | GAGAACUC U GAGUGCAU | 1235 | AUGCACUC CUGAUGAGGCCGUUAGGCCGAA IAGUUCUC | 5012 |
| 1852 | CUGAGUGC A UACAGUGC | 1236 | GCACUGUA CUGAUGAGGCCGUUAGGCCGAA ICACUCAG | 5013 |
| 1856 | GUGCAUAC A GUGCCACC | 1237 | GGUGGCAC CUGAUGAGGCCGUUAGGCCGAA IUAUGCAC | 5014 |
| 1861 | UACAGUGC C ACCCAGAG | 1238 | CUCUGGGU CUGAUGAGGCCGUUAGGCCGAA ICACUGUA | 5015 |
| 1862 | ACAGUGCC A CCCAGAGU | 1239 | ACUCUGGG CUGAUGAGGCCGUUAGGCCGAA IGCACUGU | 5016 |
| 1864 | AGUGCCAC C CAGAGUGC | 1240 | GCACUCUG CUGAUGAGGCCGUUAGGCCGAA IUGGCACU | 5017 |
| 1865 | GUGCCACC C AGAGUGCC | 1241 | GGCACUCU CUGAUGAGGCCGUUAGGCCGAA IGUGGCAC | 5018 |
| 1866 | UGCCACCC A GAGUGCCU | 1242 | AGGCACUC CUGAUGAGGCCGUUAGGCCGAA IGGUGGCA | 5019 |
| 1873 | CAGAGUGC C UGCCUCAG | 1243 | CUGAGGCA CUGAUGAGGCCGUUAGGCCGAA ICACUCUG | 5020 |
| 1874 | AGAGUGCC U GCCUCAGG | 1244 | CCUGAGGC CUGAUGAGGCCGUUAGGCCGAA IGCACUCU | 5021 |
| 1877 | GUGCCUGC C UCAGGCCA | 1245 | UGGCCUGA CUGAUGAGGCCGUUAGGCCGAA ICAGGCAC | 5022 |
| 1878 | UGCCUGCC U CAGGCCAU | 1246 | AUGGCCUG CUGAUGAGGCCGUUAGGCCGAA IGCAGGCA | 5023 |
| 1880 | CCUGCCUC A GGCCAUGA | 1247 | UCAUGGCC CUGAUGAGGCCGUUAGGCCGAA IAGGCAGG | 5024 |
| 1884 | CCUCAGGC C AUGAACAU | 1248 | AUGUUCAU CUGAUGAGGCCGUUAGGCCGAA ICCUGAGG | 5025 |
| 1885 | CUCAGGCC A UGAACAUC | 1249 | GAUGUUCA CUGAUGAGGCCGUUAGGCCGAA IGCCUGAG | 5026 |
| 1891 | CCAUGAAC A UCACCUGC | 1250 | GCAGGUGA CUGAUGAGGCCGUUAGGCCGAA IUUCAUGG | 5027 |
| 1894 | UGAACAUC A CCUGCACA | 1251 | UGUGCAGG CUGAUGAGGCCGUUAGGCCGAA IAUGUUCA | 5028 |
| 1896 | AACAUCAC C UGCACAGG | 1252 | CCUGUGCA CUGAUGAGGCCGUUAGGCCGAA IUGAUGUU | 5029 |
| 1897 | ACAUCACC U GCACAGGA | 1253 | UCCUGUGC CUGAUGAGGCCGUUAGGCCGAA IGUGAUGU | 5030 |
| 1900 | UCACCUGC A CAGGACGG | 1254 | CCGUCCUG CUGAUGAGGCCGUUAGGCCGAA ICAGGUGA | 5031 |
| 1902 | ACCUGCAC A GGACGGGG | 1255 | CCCCGUCC CUGAUGAGGCCGUUAGGCCGAA IUGCAGGU | 5032 |
| 1913 | ACGGGGAC C AGACAACU | 1256 | AGUUGUCU CUGAUGAGGCCGUUAGGCCGAA IUCCCCGU | 5033 |
| 1914 | CGGGGACC A GACAACUG | 1257 | CAGUUGUC CUGAUGAGGCCGUUAGGCCGAA IGUCCCCG | 5034 |
| 1918 | GACCAGAC A ACUGUAUC | 1258 | GAUACAGU CUGAUGAGGCCGUUAGGCCGAA IUCUGGUC | 5035 |
| 1921 | CAGACAAC U GUAUCCAG | 1259 | CUGGAUAC CUGAUGAGGCCGUUAGGCCGAA IUUGUCUG | 5036 |
| 1927 | ACUGUAUC C AGUGUGCC | 1260 | GGCACACU CUGAUGAGGCCGUUAGGCCGAA IAUACAGU | 5037 |
| 1928 | CUGUAUCC A GUGUGCCC | 1261 | GGGCACAC CUGAUGAGGCCGUUAGGCCGAA IGAUACAG | 5038 |
| 1935 | CAGUGUGC C CACUACAU | 1262 | AUGUAGUG CUGAUGAGGCCGUUAGGCCGAA ICACACUG | 5039 |
| 1936 | AGUGUGCC C ACUACAUU | 1263 | AAUGUAGU CUGAUGAGGCCGUUAGGCCGAA IGCACACU | 5040 |
| 1937 | GUGUGCCC A CUACAUUG | 1264 | CAAUGUAG CUGAUGAGGCCGUUAGGCCGAA IGGCACAC | 5041 |
| 1939 | GUGCCCAC U ACAUUGAC | 1265 | GUCAAUGU CUGAUGAGGCCGUUAGGCCGAA IUGGGCAC | 5042 |
| 1942 | CCCACUAC A UUGACGGC | 1266 | GCCGUCAA CUGAUGAGGCCGUUAGGCCGAA IUAGUGGG | 5043 |
| 1951 | UUGACGGC C CCCACUGC | 1267 | GCAGUGGG CUGAUGAGGCCGUUAGGCCGAA ICCGUCAA | 5044 |
| 1952 | UGACGGCC C CCACUGCG | 1268 | CGCAGUGG CUGAUGAGGCCGUUAGGCCGAA IGCCGUCA | 5045 |
| 1953 | GACGGCCC C CACUGCGU | 1269 | ACGCAGUG CUGAUGAGGCCGUUAGGCCGAA IGGCCGUC | 5046 |
| 1954 | ACGGCCCC C ACUGCGUC | 1270 | GACGCAGU CUGAUGAGGCCGUUAGGCCGAA IGGGCCGU | 5047 |
| 1955 | CGGCCCCC A CUGCGUCA | 1271 | UGACGCAG CUGAUGAGGCCGUUAGGCCGAA IGGGGCCG | 5048 |
| 1957 | GCCCCCAC U GCGUCAAG | 1272 | CUUGACGC CUGAUGAGGCCGUUAGGCCGAA IUGGGGGC | 5049 |
| 1963 | ACUGCGUC A AGACCUGC | 1273 | GCAGGUCU CUGAUGAGGCCGUUAGGCCGAA IACGCAGU | 5050 |
| 1968 | GUCAAGAC C UGCCCGGC | 1274 | GCCGGGCA CUGAUGAGGCCGUUAGGCCGAA IUCUUGAC | 5051 |
| 1969 | UCAAGACC U GCCCGGCA | 1275 | UGCCGGGC CUGAUGAGGCCGUUAGGCCGAA IGUCUUGA | 5052 |
| 1972 | AGACCUGC C CGGCAGGA | 1276 | UCCUGCCG CUGAUGAGGCCGUUAGGCCGAA ICAGGUCU | 5053 |
| 1973 | GACCUGCC C GGCAGGAG | 1277 | CUCCUGCC CUGAUGAGGCCGUUAGGCCGAA IGCAGGUC | 5054 |
| 1977 | UGCCCGGC A GGAGUCAU | 1278 | AUGACUCC CUGAUGAGGCCGUUAGGCCGAA ICCGGGCA | 5055 |
| 1984 | CAGGAGUC A UGGGAGAA | 1279 | UUCUCCCA CUGAUGAGGCCGUUAGGCCGAA IACUCCUG | 5056 |
| 1996 | GAGAAAAC A ACACCCUG | 1280 | CAGGGUGU CUGAUGAGGCCGUUAGGCCGAA IUUUUCUC | 5057 |
| 1999 | AAAACAAC A CCCUGGUC | 1281 | GACCAGGG CUGAUGAGGCCGUUAGGCCGAA IUUGUUUU | 5058 |
| 2001 | AACAACAC C CUGGUCUG | 1282 | CAGACCAG CUGAUGAGGCCGUUAGGCCGAA IUGUUGUU | 5059 |
| 2002 | ACAACACC C UGGUCUGG | 1283 | CCAGACCA CUGAUGAGGCCGUUAGGCCGAA IGUGUUGU | 5060 |
| 2003 | CAACACCC U GGUCUGGA | 1284 | UCCAGACC CUGAUGAGGCCGUUAGGCCGAA IGGUGUUG | 5061 |
| 2008 | CCCUGGUC U GGAAGUAC | 1285 | GUACUUCC CUGAUGAGGCCGUUAGGCCGAA IACCAGGG | 5062 |
| 2019 | AAGUACGC A GACGCCGG | 1286 | CCGGCGUC CUGAUGAGGCCGUUAGGCCGAA ICGUACUU | 5063 |
| 2025 | GCAGACGC C GGCCAUGU | 1287 | ACAUGGCC CUGAUGAGGCCGUUAGGCCGAA ICGUCUGC | 5064 |
| 2029 | ACGCCGGC C AUGUGUGC | 1288 | GCACACAU CUGAUGAGGCCGUUAGGCCGAA ICCGGCGU | 5065 |
| 2030 | CGCCGGCC A UGUGUGCC | 1289 | GGCACACA CUGAUGAGGCCGUUAGGCCGAA IGCCGGCG | 5066 |
| 2038 | AUGUGUGC C ACCUGUGC | 1290 | GCACAGGU CUGAUGAGGCCGUUAGGCCGAA ICACACAU | 5067 |
| 2039 | UGUGUGCC A CCUGUGCC | 1291 | GGCACAGG CUGAUGAGGCCGUUAGGCCGAA IGCACACA | 5068 |
| 2041 | UGUGCCAC C UGUGCCAU | 1292 | AUGGCACA CUGAUGAGGCCGUUAGGCCGAA IUGGCACA | 5069 |
| 2042 | GUGCCACC U GUGCCAUC | 1293 | GAUGGCAC CUGAUGAGGCCGUUAGGCCGAA IGUGGCAC | 5070 |
| 2047 | ACCUGUGC C AUCCAAAC | 1294 | GUUUGGAU CUGAUGAGGCCGUUAGGCCGAA ICACAGGU | 5071 |
| 2048 | CCUGUGCC A UCCAAACU | 1295 | AGUUUGGA CUGAUGAGGCCGUUAGGCCGAA IGCACAGG | 5072 |
| 2051 | GUGCCAUC C AAACUGCA | 1296 | UGCAGUUU CUGAUGAGGCCGUUAGGCCGAA IAUGGCAC | 5073 |
| 2052 | UGCCAUCC A AACUGCAC | 1297 | GUGCAGUU CUGAUGAGGCCGUUAGGCCGAA IGAUGGCA | 5074 |
| 2056 | AUCCAAAC U GCACCUAC | 1298 | GUAGGUGC CUGAUGAGGCCGUUAGGCCGAA IUUUGGAU | 5075 |
| 2059 | CAAACUGC A CCUACGGA | 1299 | UCCGUAGG CUGAUGAGGCCGUUAGGCCGAA ICAGUUUG | 5076 |
| 2061 | AACUGCAC C UACGGAUG | 1300 | CAUCCGUA CUGAUGAGGCCGUUAGGCCGAA IUGCAGUU | 5077 |
| 2062 | ACUGCACC U ACGGAUGC | 1301 | GCAUCCGU CUGAUGAGGCCGUUAGGCCGAA IGUGCAGU | 5078 |
| 2071 | ACGGAUGC A CUGGGCCA | 1302 | UGGCCCAG CUGAUGAGGCCGUUAGGCCGAA ICAUCCGU | 5079 |
| 2073 | GGAUGCAC U GGGCCAGG | 1303 | CCUGGCCC CUGAUGAGGCCGUUAGGCCGAA IUGCAUCC | 5080 |
| 2078 | CACUGGGC C AGGUCUUG | 1304 | CAAGACCU CUGAUGAGGCCGUUAGGCCGAA ICCCAGUG | 5081 |
| 2079 | ACUGGGCC A GGUCUUGA | 1305 | UCAAGACC CUGAUGAGGCCGUUAGGCCGAA IGCCCAGU | 5082 |
| 2084 | GCCAGGUC U UGAAGGCU | 1306 | AGCCUUCA CUGAUGAGGCCGUUAGGCCGAA IACCUGGC | 5083 |
| 2092 | UUGAAGGC U GUCCAACG | 1307 | CGUUGGAC CUGAUGAGGCCGUUAGGCCGAA ICCUUCAA | 5084 |
| 2096 | AGGCUGUC C AACGAAUG | 1308 | CAUUCGUU CUGAUGAGGCCGUUAGGCCGAA IACAGCCU | 5085 |
| 2097 | GGCUGUCC A ACGAAUGG | 1309 | CCAUUCGU CUGAUGAGGCCGUUAGGCCGAA IGACAGCC | 5086 |
| 2108 | GAAUGGGC C UAAGAUCC | 1310 | GGAUCUUA CUGAUGAGGCCGUUAGGCCGAA ICCCAUUC | 5087 |
| 2109 | AAUGGGCC U AAGAUCCC | 1311 | GGGAUCUU CUGAUGAGGCCGUUAGGCCGAA IGCCCAUU | 5088 |
| 2116 | CUAAGAUC C CGUCCAUC | 1312 | GAUGGACG CUGAUGAGGCCGUUAGGCCGAA IAUCUUAG | 5089 |
| 2117 | UAAGAUCC C GUCCAUCG | 1313 | CGAUGGAC CUGAUGAGGCCGUUAGGCCGAA IGAUCUUA | 5090 |
| 2121 | AUCCCGUC C AUCGCCAC | 1314 | GUGGCGAU CUGAUGAGGCCGUUAGGCCGAA IACGGGAU | 5091 |
| 2122 | UCCCGUCC A UCGCCACU | 1315 | AGUGGCGA CUGAUGAGGCCGUUAGGCCGAA IGACGGGA | 5092 |
| 2127 | UCCAUCGC C ACUGGGAU | 1316 | AUCCCAGU CUGAUGAGGCCGUUAGGCCGAA ICGAUGGA | 5093 |
| 2128 | CCAUCGCC A CUGGGAUG | 1317 | CAUCCCAG CUGAUGAGGCCGUUAGGCCGAA IGCGAUGG | 5094 |
| 2130 | AUCGCCAC U GGGAUGGU | 1318 | ACCAUCCC CUGAUGAGGCCGUUAGGCCGAA IUGGCGAU | 5095 |
| 2145 | GUGGGGGC C CUCCUCUU | 1319 | AAGAGGAG CUGAUGAGGCCGUUAGGCCGAA ICCCCCAC | 5096 |
| 2146 | UGGGGGCC C UCCUCUUG | 1320 | CAAGAGGA CUGAUGAGGCCGUUAGGCCGAA IGCCCCCA | 5097 |
| 2147 | GGGGGCCC U CCUCUUGC | 1321 | GCAAGAGG CUGAUGAGGCCGUUAGGCCGAA IGGCCCCC | 5098 |
| 2149 | GGGCCCUC C UCUUGCUG | 1322 | CAGCAAGA CUGAUGAGGCCGUUAGGCCGAA IAGGGCCC | 5099 |
| 2150 | GGCCCUCC U CUUGCUGC | 1323 | GCAGCAAG CUGAUGAGGCCGUUAGGCCGAA IGAGGGCC | 5100 |
| 2152 | CCCUCCUC U UGCUGCUG | 1324 | CAGCAGCA CUGAUGAGGCCGUUAGGCCGAA IAGGAGGG | 5101 |
| 2156 | CCUCUUGC U GCUGGUGG | 1325 | CCACCAGC CUGAUGAGGCCGUUAGGCCGAA ICAAGAGG | 5102 |
| 2159 | CUUGCUGC U GGUGGUGG | 1326 | CCACCACC CUGAUGAGGCCGUUAGGCCGAA ICAGCAAG | 5103 |
| 2169 | GUGGUGGC C CUGGGGAU | 1327 | AUCCCCAG CUGAUGAGGCCGUUAGGCCGAA ICCACCAC | 5104 |
| 2170 | UGGUGGCC C UGGGGAUC | 1328 | GAUCCCCA CUGAUGAGGCCGUUAGGCCGAA IGCCACCA | 5105 |
| 2171 | GGUGGCCC U GGGGAUCG | 1329 | CGAUCCCC CUGAUGAGGCCGUUAGGCCGAA IGGCCACC | 5106 |
| 2182 | GGAUCGGC C UCUUCAUG | 1330 | CAUGAAGA CUGAUGAGGCCGUUAGGCCGAA ICCGAUCC | 5107 |
| 2183 | GAUCGGCC U CUUCAUGC | 1331 | GCAUGAAG CUGAUGAGGCCGUUAGGCCGAA IGCCGAUC | 5108 |
| 2185 | UCGGCCUC U UCAUGCGA | 1332 | UCGCAUGA CUGAUGAGGCCGUUAGGCCGAA IAGGCCGA | 5109 |
| 2188 | GCCUCUUC A UGCGAAGG | 1333 | CCUUCGCA CUGAUGAGGCCGUUAGGCCGAA IAAGAGGC | 5110 |
| 2200 | GAAGGCGC C ACAUCGUU | 1334 | AACGAUGU CUGAUGAGGCCGUUAGGCCGAA ICGCCUUC | 5111 |
| 2201 | AAGGCGCC A CAUCGUUC | 1335 | GAACGAUG CUGAUGAGGCCGUUAGGCCGAA IGCGCCUU | 5112 |
| 2203 | GGCGCCAC A UCGUUCGG | 1336 | CCGAACGA CUGAUGAGGCCGUUAGGCCGAA IUGGCGCC | 5113 |
| 2218 | GGAAGCGC A CGCUGCGG | 1337 | CCGCAGCG CUGAUGAGGCCGUUAGGCCGAA ICGCUUCC | 5114 |
| 2222 | GCGCACGC U GCGGAGGC | 1338 | GCCUCCGC CUGAUGAGGCCGUUAGGCCGAA ICGUGCGC | 5115 |
| 2231 | GCGGAGGC U GCUGCAGG | 1339 | CCUGCAGC CUGAUGAGGCCGUUAGGCCGAA ICCUCCGC | 5116 |
| 2234 | GAGGCUGC U GCAGGAGA | 1340 | UCUCCUGC CUGAUGAGGCCGUUAGGCCGAA ICAGCCUC | 5117 |
| 2237 | GCUGCUGC A GGAGAGGG | 1341 | CCCUCUCC CUGAUGAGGCCGUUAGGCCGAA ICAGCAGC | 5118 |
| 2249 | GAGGGAGC U UGUGGAGC | 1342 | GCUCCACA CUGAUGAGGCCGUUAGGCCGAA ICUCCCUC | 5119 |
| 2258 | UGUGGAGC C UCUUACAC | 1343 | GUGUAAGA CUGAUGAGGCCGUUAGGCCGAA ICUCCACA | 5120 |
| 2259 | GUGGAGCC U CUUACACC | 1344 | GGUGUAAG CUGAUGAGGCCGUUAGGCCGAA IGCUCCAC | 5121 |
| 2261 | GGAGCCUC U UACACCCA | 1345 | UGGGUGUA CUGAUGAGGCCGUUAGGCCGAA IAGGCUCC | 5122 |
| 2265 | CCUCUUAC A CCCAGUGG | 1346 | CCACUGGG CUGAUGAGGCCGUUAGGCCGAA IUAAGAGG | 5123 |
| 2267 | UCUUACAC C CAGUGGAG | 1347 | CUCCACUG CUGAUGAGGCCGUUAGGCCGAA IUGUAAGA | 5124 |
| 2268 | CUUACACC C AGUGGAGA | 1348 | UCUCCACU CUGAUGAGGCCGUUAGGCCGAA IGUGUAAG | 5125 |
| 2269 | UUACACCC A GUGGAGAA | 1349 | UUCUCCAC CUGAUGAGGCCGUUAGGCCGAA IGGUGUAA | 5126 |
| 2280 | GGAGAAGC U CCCAACCA | 1350 | UGGUUGGG CUGAUGAGGCCGUUAGGCCGAA ICUUCUCC | 5127 |
| 2282 | AGAAGCUC C CAACCAAG | 1351 | CUUGGUUG CUGAUGAGGCCGUUAGGCCGAA IAGCUUCU | 5128 |
| 2283 | GAAGCUCC C AACCAAGC | 1352 | GCUUGGUU CUGAUGAGGCCGUUAGGCCGAA IGAGCUUC | 5129 |
| 2284 | AAGCUCCC A ACCAAGCU | 1353 | AGCUUGGU CUGAUGAGGCCGUUAGGCCGAA IGGAGCUU | 5130 |
| 2287 | CUCCCAAC C AAGCUCUC | 1354 | GAGAGCUU CUGAUGAGGCCGUUAGGCCGAA IUUGGGAG | 5131 |
| 2288 | UCCCAACC A AGCUCUCU | 1355 | AGAGAGCU CUGAUGAGGCCGUUAGGCCGAA IGUUGGGA | 5132 |
| 2292 | AACCAAGC U CUCUUGAG | 1356 | CUCAAGAG CUGAUGAGGCCGUUAGGCCGAA ICUUGGUU | 5133 |
| 2294 | CCAAGCUC U CUUGAGGA | 1357 | UCCUCAAG CUGAUGAGGCCGUUAGGCCGAA IAGCUUGG | 5134 |
| 2296 | AAGCUCUC U UGAGGAUC | 1358 | GAUCCUCA CUGAUGAGGCCGUUAGGCCGAA IAGAGCUU | 5135 |
| 2305 | UGAGGAUC U UGAAGGAA | 1359 | UUCCUUCA CUGAUGAGGCCGUUAGGCCGAA IAUCCUCA | 5136 |
| 2316 | AAGGAAAC U GAAUUCAA | 1360 | UUGAAUUC CUGAUGAGGCCGUUAGGCCGAA IUUUCCUU | 5137 |
| 2323 | CUGAAUUC A AAAAGAUC | 1361 | GAUCUUUU CUGAUGAGGCCGUUAGGCCGAA IAAUUCAG | 5138 |
| 2332 | AAAAGAUC A AAGUGCUG | 1362 | CAGCACUU CUGAUGAGGCCGUUAGGCCGAA IAUCUUUU | 5139 |
| 2339 | CAAAGUGC U GGGCUCCG | 1363 | CGGAGCCC CUGAUGAGGCCGUUAGGCCGAA ICACUUUG | 5140 |
| 2344 | UGCUGGGC U CCGGUGCG | 1364 | CGCACCGG CUGAUGAGGCCGUUAGGCCGAA ICCCAGCA | 5141 |
| 2346 | CUGGGCUC C GGUGCGUU | 1365 | AACGCACC CUGAUGAGGCCGUUAGGCCGAA IAGCCCAG | 5142 |
| 2359 | CGUUCGGC A CGGUGUAU | 1366 | AUACACCG CUGAUGAGGCCGUUAGGCCGAA ICCGAACG | 5143 |
| 2375 | UAAGGGAC U CUGGAUCC | 1367 | GGAUCCAG CUGAUGAGGCCGUUAGGCCGAA IUCCCUUA | 5144 |
| 2377 | AGGGACUC U GGAUCCCA | 1368 | UGGGAUCC CUGAUGAGGCCGUUAGGCCGAA IAGUCCCU | 5145 |
| 2383 | UCUGGAUC C CAGAAGGU | 1369 | ACCUUCUG CUGAUGAGGCCGUUAGGCCGAA IAUCCAGA | 5146 |
| 2384 | CUGGAUCC C AGAAGGUG | 1370 | CACCUUCU CUGAUGAGGCCGUUAGGCCGAA IGAUCCAG | 5147 |
| 2385 | UGGAUCCC A GAAGGUGA | 1371 | UCACCUUC CUGAUGAGGCCGUUAGGCCGAA IGGAUCCA | 5148 |
| 2408 | UAAAAUUC C CGUCGCUA | 1372 | UAGCGACG CUGAUGAGGCCGUUAGGCCGAA IAAUUUUA | 5149 |
| 2409 | AAAAUUCC C GUCGCUAU | 1373 | AUAGCGAC CUGAUGAGGCCGUUAGGCCGAA IGAAUUUU | 5150 |
| 2415 | CCCGUCGC U AUCAAGGA | 1374 | UCCUUGAU CUGAUGAGGCCGUUAGGCCGAA ICGACGGG | 5151 |
| 2419 | UCGCUAUC A AGGAAUUA | 1375 | UAAUUCCU CUGAUGAGGCCGUUAGGCCGAA IAUAGCGA | 5152 |
| 2436 | AGAGAAGC A ACAUCUCC | 1376 | GGAGAUGU CUGAUGAGGCCGUUAGGCCGAA ICUUCUCU | 5153 |
| 2439 | GAAGCAAC A UCUCCGAA | 1377 | UUCGGAGA CUGAUGAGGCCGUUAGGCCGAA IUUGCUUC | 5154 |
| 2442 | GCAACAUC U CCGAAAGC | 1378 | GCUUUCGG CUGAUGAGGCCGUUAGGCCGAA IAUGUUGC | 5155 |
| 2444 | AACAUCUC C GAAAGCCA | 1379 | UGGCUUUC CUGAUGAGGCCGUUAGGCCGAA IAGAUGUU | 5156 |
| 2451 | CCGAAAGC C AACAAGGA | 1380 | UCCUUGUU CUGAUGAGGCCGUUAGGCCGAA ICUUUCGG | 5157 |
| 2452 | CGAAAGCC A ACAAGGAA | 1381 | UUCCUUGU CUGAUGAGGCCGUUAGGCCGAA IGCUUUCG | 5158 |
| 2455 | AAGCCAAC A AGGAAAUC | 1382 | GAUUUCCU CUGAUGAGGCCGUUAGGCCGAA IUUGGCUU | 5159 |
| 2464 | AGGAAAUC C UCGAUGAA | 1383 | UUCAUCGA CUGAUGAGGCCGUUAGGCCGAA IAUUUCCU | 5160 |
| 2465 | GGAAAUCC U CGAUGAAG | 1384 | CUUCAUCG CUGAUGAGGCCGUUAGGCCGAA IGAUUUCC | 5161 |
| 2475 | GAUGAAGC C UACGUGAU | 1385 | AUCACGUA CUGAUGAGGCCGUUAGGCCGAA ICUUCAUC | 5162 |
| 2476 | AUGAAGCC U ACGUGAUG | 1386 | CAUCACGU CUGAUGAGGCCGUUAGGCCGAA IGCUUCAU | 5163 |
| 2487 | GUGAUGGC C AGCGUGGA | 1387 | UCCACGCU CUGAUGAGGCCGUUAGGCCGAA ICCAUCAC | 5164 |
| 2488 | UGAUGGCC A GCGUGGAC | 1388 | GUCCACGC CUGAUGAGGCCGUUAGGCCGAA IGCCAUCA | 5165 |
| 2497 | GCGUGGAC A ACCCCCAC | 1389 | GUGGGGGU CUGAUGAGGCCGUUAGGCCGAA IUCCACGC | 5166 |
| 2500 | UGGACAAC C CCCACGUG | 1390 | CACGUGGG CUGAUGAGGCCGUUAGGCCGAA IUUGUCCA | 5167 |
| 2501 | GGACAACC C CCACGUGU | 1391 | ACACGUGG CUGAUGAGGCCGUUAGGCCGAA IGUUGUCC | 5168 |
| 2502 | GACAACCC C CACGUGUG | 1392 | CACACGUG CUGAUGAGGCCGUUAGGCCGAA IGGUUGUC | 5169 |
| 2503 | ACAACCCC C ACGUGUGC | 1393 | GCACACGU CUGAUGAGGCCGUUAGGCCGAA IGGGUUGU | 5170 |
| 2504 | CAACCCCC A CGUGUGCC | 1394 | GGCACACG CUGAUGAGGCCGUUAGGCCGAA IGGGGUUG | 5171 |
| 2512 | ACGUGUGC C GCCUGCUG | 1395 | CAGCAGGC CUGAUGAGGCCGUUAGGCCGAA ICACACGU | 5172 |
| 2515 | UGUGCCGC C UGCUGGGC | 1396 | GCCCAGCA CUGAUGAGGCCGUUAGGCCGAA ICGGCACA | 5173 |
| 2516 | GUGCCGCC U GCUGGGCA | 1397 | UGCCCAGC CUGAUGAGGCCGUUAGGCCGAA IGCGGCAC | 5174 |
| 2519 | CCGCCUGC U GGGCAUCU | 1398 | AGAUGCCC CUGAUGAGGCCGUUAGGCCGAA ICAGGCGG | 5175 |
| 2524 | UGCUGGGC A UCUGCCUC | 1399 | GAGGCAGA CUGAUGAGGCCGUUAGGCCGAA ICCCAGCA | 5176 |
| 2527 | UGGGCAUC U GCCUCACC | 1400 | GGUGAGGC CUGAUGAGGCCGUUAGGCCGAA IAUGCCCA | 5177 |
| 2530 | GCAUCUGC C UCACCUCC | 1401 | GGAGGUGA CUGAUGAGGCCGUUAGGCCGAA ICAGAUGC | 5178 |
| 2531 | CAUCUGCC U CACCUCCA | 1402 | UGGAGGUG CUGAUGAGGCCGUUAGGCCGAA IGCAGAUG | 5179 |
| 2533 | UCUGCCUC A CCUCCACC | 1403 | GGUGGAGG CUGAUGAGGCCGUUAGGCCGAA IAGGCAGA | 5180 |
| 2535 | UGCCUCAC C UCCACCGU | 1404 | ACGGUGGA CUGAUGAGGCCGUUAGGCCGAA IUGAGGCA | 5181 |
| 2536 | GCCUCACC U CCACCGUG | 1405 | CACGGUGG CUGAUGAGGCCGUUAGGCCGAA IGUGAGGC | 5182 |
| 2538 | CUCACCUC C ACCGUGCA | 1406 | UGCACGGU CUGAUGAGGCCGUUAGGCCGAA IAGGUGAG | 5183 |
| 2539 | UCACCUCC A CCGUGCAA | 1407 | UUGCACGG CUGAUGAGGCCGUUAGGCCGAA IGAGGUGA | 5184 |
| 2541 | ACCUCCAC C GUGCAACU | 1408 | AGUUGCAC CUGAUGAGGCCGUUAGGCCGAA IUGGAGGU | 5185 |
| 2546 | CACCGUGC A ACUCAUCA | 1409 | UGAUGAGU CUGAUGAGGCCGUUAGGCCGAA ICACGGUG | 5186 |
| 2549 | CGUGCAAC U CAUCACGC | 1410 | GCGUGAUG CUGAUGAGGCCGUUAGGCCGAA IUUGCACG | 5187 |
| 2551 | UGCAACUC A UCACGCAG | 1411 | CUGCGUGA CUGAUGAGGCCGUUAGGCCGAA IAGUUGCA | 5188 |
| 2554 | AACUCAUC A CGCAGCUC | 1412 | GAGCUGCG CUGAUGAGGCCGUUAGGCCGAA IAUGAGUU | 5189 |
| 2558 | CAUCACGC A GCUCAUGC | 1413 | GCAUGAGC CUGAUGAGGCCGUUAGGCCGAA ICGUGAUG | 5190 |
| 2561 | CACGCAGC U CAUGCCCU | 1414 | AGGGCAUG CUGAUGAGGCCGUUAGGCCGAA ICUGCGUG | 5191 |
| 2563 | CGCAGCUC A UGCCCUUC | 1415 | GAAGGGCA CUGAUGAGGCCGUUAGGCCGAA IAGCUGCG | 5192 |
| 2567 | GCUCAUGC C CUUCGGCU | 1416 | AGCCGAAG CUGAUGAGGCCGUUAGGCCGAA ICAUGAGC | 5193 |
| 2568 | CUCAUGCC C UUCGGCUG | 1417 | CAGCCGAA CUGAUGAGGCCGUUAGGCCGAA IGCAUGAG | 5194 |
| 2569 | UCAUGCCC U UCGGCUGC | 1418 | GCAGCCGA CUGAUGAGGCCGUUAGGCCGAA IGGCAUGA | 5195 |
| 2575 | CCUUCGGC U GCCUCCUG | 1419 | CAGGAGGC CUGAUGAGGCCGUUAGGCCGAA ICCGAAGG | 5196 |
| 2578 | UCGGCUGC C UCCUGGAC | 1420 | GUCCAGGA CUGAUGAGGCCGUUAGGCCGAA ICAGCCGA | 5197 |
| 2579 | CGGCUGCC U CCUGGACU | 1421 | AGUCCAGG CUGAUGAGGCCGUUAGGCCGAA IGCAGCCG | 5198 |
| 2581 | GCUGCCUC C UGGACUAU | 1422 | AUAGUCCA CUGAUGAGGCCGUUAGGCCGAA IAGGCAGC | 5199 |
| 2582 | CUGCCUCC U GGACUAUG | 1423 | CAUAGUCC CUGAUGAGGCCGUUAGGCCGAA IGAGGCAG | 5200 |
| 2587 | UCCUGGAC U AUGUCCGG | 1424 | CCGGACAU CUGAUGAGGCCGUUAGGCCGAA IUCCAGGA | 5201 |
| 2593 | ACUAUGUC C GGGAACAC | 1425 | GUGUUCCC CUGAUGAGGCCGUUAGGCCGAA IACAUAGU | 5202 |
| 2600 | CCGGGAAC A CAAAGACA | 1426 | UGUCUUUG CUGAUGAGGCCGUUAGGCCGAA IUUCCCGG | 5203 |
| 2602 | GGGAACAC A AAGACAAU | 1427 | AUUGUCUU CUGAUGAGGCCGUUAGGCCGAA IUGUUCCC | 5204 |
| 2608 | ACAAAGAC A AUAUUGGC | 1428 | GCCAAUAU CUGAUGAGGCCGUUAGGCCGAA IUCUUUGU | 5205 |
| 2617 | AUAUUGGC U CCCAGUAC | 1429 | GUACUGGG CUGAUGAGGCCGUUAGGCCGAA ICCAAUAU | 5206 |
| 2619 | AUUGGCUC C CAGUACCU | 1430 | AGGUACUG CUGAUGAGGCCGUUAGGCCGAA IAGCCAAU | 5207 |
| 2620 | UUGGCUCC C AGUACCUG | 1431 | CAGGUACU CUGAUGAGGCCGUUAGGCCGAA IGAGCCAA | 5208 |
| 2621 | UGGCUCCC A GUACCUGC | 1432 | GCAGGUAC CUGAUGAGGCCGUUAGGCCGAA IGGAGCCA | 5209 |
| 2626 | CCCAGUAC C UGCUCAAC | 1433 | GUUGAGCA CUGAUGAGGCCGUUAGGCCGAA IUACUGGG | 5210 |
| 2627 | CCAGUACC U GCUCAACU | 1434 | AGUUGAGC CUGAUGAGGCCGUUAGGCCGAA IGUACUGG | 5211 |
| 2630 | GUACCUGC U CAACUGGU | 1435 | ACCAGUUG CUGAUGAGGCCGUUAGGCCGAA ICAGGUAC | 5212 |
| 2632 | ACCUGCUC A ACUGGUGU | 1436 | ACACCAGU CUGAUGAGGCCGUUAGGCCGAA IAGCAGGU | 5213 |
| 2635 | UGCUCAAC U GGUGUGUG | 1437 | CACACACC CUGAUGAGGCCGUUAGGCCGAA IUUGAGCA | 5214 |
| 2645 | GUGUGUGC A GAUCGCAA | 1438 | UUGCGAUC CUGAUGAGGCCGUUAGGCCGAA ICACACAC | 5215 |
| 2652 | CAGAUCGC A AAGGGCAU | 1439 | AUGCCCUU CUGAUGAGGCCGUUAGGCCGAA ICGAUCUG | 5216 |
| 2659 | CAAAGGGC A UGAACUAC | 1440 | GUAGUUCA CUGAUGAGGCCGUUAGGCCGAA ICCCUUUG | 5217 |
| 2665 | GCAUGAAC U ACUUGGAG | 1441 | CUCCAAGU CUGAUGAGGCCGUUAGGCCGAA IUUCAUGC | 5218 |
| 2668 | UGAACUAC U UGGAGGAC | 1442 | GUCCUCCA CUGAUGAGGCCGUUAGGCCGAA IUAGUUCA | 5219 |
| 2677 | UGGAGGAC C GUCGCUUG | 1443 | CAAGCGAC CUGAUGAGGCCGUUAGGCCGAA IUCCUCCA | 5220 |
| 2683 | ACCGUCGC U UGGUGCAC | 1444 | GUGCACCA CUGAUGAGGCCGUUAGGCCGAA ICGACGGU | 5221 |
| 2690 | CUUGGUGC A CCGCGACC | 1445 | GGUCGCGG CUGAUGAGGCCGUUAGGCCGAA ICACCAAG | 5222 |
| 2692 | UGGUGCAC C GCGACCUG | 1446 | CAGGUCGC CUGAUGAGGCCGUUAGGCCGAA IUGCACCA | 5223 |
| 2698 | ACCGCGAC C UGGCAGCC | 1447 | GGCUGCCA CUGAUGAGGCCGUUAGGCCGAA IUCGCGGU | 5224 |
| 2699 | CCGCGACC U GGCAGCCA | 1448 | UGGCUGCC CUGAUGAGGCCGUUAGGCCGAA IGUCGCGG | 5225 |
| 2703 | GACCUGGC A GCCAGGAA | 1449 | UUCCUGGC CUGAUGAGGCCGUUAGGCCGAA ICCAGGUC | 5226 |
| 2706 | CUGGCAGC C AGGAACGU | 1450 | ACGUUCCU CUGAUGAGGCCGUUAGGCCGAA ICUGCCAG | 5227 |
| 2707 | UGGCAGCC A GGAACGUA | 1451 | UACGUUCC CUGAUGAGGCCGUUAGGCCGAA IGCUGCCA | 5228 |
| 2717 | GAACGUAC U GGUGAAAA | 1452 | UUUUCACC CUGAUGAGGCCGUUAGGCCGAA IUACGUUC | 5229 |
| 2727 | GUGAAAAC A CCGCAGCA | 1453 | UGCUGCGG CUGAUGAGGCCGUUAGGCCGAA IUUUUCAC | 5230 |
| 2729 | GAAAACAC C GCAGCAUG | 1454 | CAUGCUGC CUGAUGAGGCCGUUAGGCCGAA IUGUUUUC | 5231 |
| 2732 | AACACCGC A GCAUGUCA | 1455 | UGACAUGC CUGAUGAGGCCGUUAGGCCGAA ICGGUGUU | 5232 |
| 2735 | ACCGCAGC A UGUCAAGA | 1456 | UCUUGACA CUGAUGAGGCCGUUAGGCCGAA ICUGCGGU | 5233 |
| 2740 | AGCAUGUC A AGAUCACA | 1457 | UGUGAUCU CUGAUGAGGCCGUUAGGCCGAA IACAUGCU | 5234 |
| 2746 | UCAAGAUC A CAGAUUUU | 1458 | AAAAUCUG CUGAUGAGGCCGUUAGGCCGAA IAUCUUGA | 5235 |
| 2748 | AAGAUCAC A GAUUUUGG | 1459 | CCAAAAUC CUGAUGAGGCCGUUAGGCCGAA IUGAUCUU | 5236 |
| 2759 | UUUUGGGC U GGCCAAAC | 1460 | GUUUGGCC CUGAUGAGGCCGUUAGGCCGAA ICCCAAAA | 5237 |
| 2763 | GGGCUGGC C AAACUGCU | 1461 | AGCAGUUU CUGAUGAGGCCGUUAGGCCGAA ICCAGCCC | 5238 |
| 2764 | GGCUGGCC A AACUGCUG | 1462 | CAGCAGUU CUGAUGAGGCCGUUAGGCCGAA IGCCAGCC | 5239 |
| 2768 | GGCCAAAC U GCUGGGUG | 1463 | CACCCAGC CUGAUGAGGCCGUUAGGCCGAA IUUUGGCC | 5240 |
| 2771 | CAAACUGC U GGGUGCGG | 1464 | CCGCACCC CUGAUGAGGCCGUUAGGCCGAA ICAGUUUG | 5241 |
| 2794 | AAGAAUAC C AUGCAGAA | 1465 | UUCUGCAU CUGAUGAGGCCGUUAGGCCGAA IUAUUCUU | 5242 |
| 2795 | AGAAUACC A UGCAGAAG | 1466 | CUUCUGCA CUGAUGAGGCCGUUAGGCCGAA IGUAUUCU | 5243 |
| 2799 | UACCAUGC A GAAGGAGG | 1467 | CCUCCUUC CUGAUGAGGCCGUUAGGCCGAA ICAUGGUA | 5244 |
| 2809 | AAGGAGGC A AAGUGCCU | 1468 | AGGCACUU CUGAUGAGGCCGUUAGGCCGAA ICCUCCUU | 5245 |
| 2816 | CAAAGUGC C UAUCAAGU | 1469 | ACUUGAUA CUGAUGAGGCCGUUAGGCCGAA ICACUUUG | 5246 |
| 2817 | AAAGUGCC U AUCAAGUG | 1470 | CACUUGAU CUGAUGAGGCCGUUAGGCCGAA IGCACUUU | 5247 |
| 2821 | UGCCUAUC A AGUGGAUG | 1471 | CAUCCACU CUGAUGAGGCCGUUAGGCCGAA IAUAGGCA | 5248 |
| 2832 | UGGAUGGC A UUGGAAUC | 1472 | GAUUCCAA CUGAUGAGGCCGUUAGGCCGAA ICCAUCCA | 5249 |
| 2841 | UUGGAAUC A AUUUUACA | 1473 | UGUAAAAU CUGAUGAGGCCGUUAGGCCGAA IAUUCCAA | 5250 |
| 2849 | AAUUUUAC A CAGAAUCU | 1474 | AGAUUCUG CUGAUGAGGCCGUUAGGCCGAA IUAAAAUU | 5251 |
| 2851 | UUUUACAC A GAAUCUAU | 1475 | AUAGAUUC CUGAUGAGGCCGUUAGGCCGAA IUGUAAAA | 5252 |
| 2857 | ACAGAAUC U AUACCCAC | 1476 | GUGGGUAU CUGAUGAGGCCGUUAGGCCGAA IAUUCUGU | 5253 |
| 2862 | AUCUAUAC C CACCAGAG | 1477 | CUCUGGUG CUGAUGAGGCCGUUAGGCCGAA IUAUAGAU | 5254 |
| 2863 | UCUAUACC C ACCAGAGU | 1478 | ACUCUGGU CUGAUGAGGCCGUUAGGCCGAA IGUAUAGA | 5255 |
| 2864 | CUAUACCC A CCAGAGUG | 1479 | CACUCUGG CUGAUGAGGCCGUUAGGCCGAA IGGUAUAG | 5256 |
| 2866 | AUACCCAC C AGAGUGAU | 1480 | AUCACUCU CUGAUGAGGCCGUUAGGCCGAA IUGGGUAU | 5257 |
| 2867 | UACCCACC A GAGUGAUG | 1481 | CAUCACUC CUGAUGAGGCCGUUAGGCCGAA IGUGGGUA | 5258 |
| 2878 | GUGAUGUC U GGAGCUAC | 1482 | GUAGCUCC CUGAUGAGGCCGUUAGGCCGAA IACAUCAC | 5259 |
| 2884 | UCUGGAGC U ACGGGGUG | 1483 | CACCCCGU CUGAUGAGGCCGUUAGGCCGAA ICUCCAGA | 5260 |
| 2895 | GGGGUGAC C GUUUGGGA | 1484 | UCCCAAAC CUGAUGAGGCCGUUAGGCCGAA IUCACCCC | 5261 |
| 2913 | UUGAUGAC C UUUGGAUC | 1485 | GAUCCAAA CUGAUGAGGCCGUUAGGCCGAA IUCAUCAA | 5262 |
| 2914 | UGAUGACC U UUGGAUCC | 1486 | GGAUCCAA CUGAUGAGGCCGUUAGGCCGAA IGUCAUCA | 5263 |
| 2922 | UUUGGAUC C AAGCCAUA | 1487 | UAUGGCUU CUGAUGAGGCCGUUAGGCCGAA IAUCCAAA | 5264 |
| 2923 | UUGGAUCC A AGCCAUAU | 1488 | AUAUGGCU CUGAUGAGGCCGUUAGGCCGAA IGAUCCAA | 5265 |
| 2927 | AUCCAAGC C AUAUGACG | 1489 | CGUCAUAU CUGAUGAGGCCGUUAGGCCGAA ICUUGGAU | 5266 |
| 2928 | UCCAAGCC A UAUGACGG | 1490 | CCGUCAUA CUGAUGAGGCCGUUAGGCCGAA IGCUUGGA | 5267 |
| 2941 | ACGGAAUC C CUGCCAGC | 1491 | GCUGGCAG CUGAUGAGGCCGUUAGGCCGAA IAUUCCGU | 5268 |
| 2942 | CGGAAUCC C UGCCAGCG | 1492 | CGCUGGCA CUGAUGAGGCCGUUAGGCCGAA IGAUUCCG | 5269 |
| 2943 | GGAAUCCC U GCCAGCGA | 1493 | UCGCUGGC CUGAUGAGGCCGUUAGGCCGAA IGGAUUCC | 5270 |
| 2946 | AUCCCUGC C AGCGAGAU | 1494 | AUCUCGCU CUGAUGAGGCCGUUAGGCCGAA ICAGGGAU | 5271 |
| 2947 | UCCCUGCC A GCGAGAUC | 1495 | GAUCUCGC CUGAUGAGGCCGUUAGGCCGAA IGCAGGGA | 5272 |
| 2956 | GCGAGAUC U CCUCCAUC | 1496 | GAUGGAGG CUGAUGAGGCCGUUAGGCCGAA IAUCUCGC | 5273 |
| 2958 | GAGAUCUC C UCCAUCCU | 1497 | AGGAUGGA CUGAUGAGGCCGUUAGGCCGAA IAGAUCUC | 5274 |
| 2959 | AGAUCUCC U CCAUCCUG | 1498 | CAGGAUGG CUGAUGAGGCCGUUAGGCCGAA IGAGAUCU | 5275 |
| 2961 | AUCUCCUC C AUCCUGGA | 1499 | UCCAGGAU CUGAUGAGGCCGUUAGGCCGAA IAGGAGAU | 5276 |
| 2962 | UCUCCUCC A UCCUGGAG | 1500 | CUCCAGGA CUGAUGAGGCCGUUAGGCCGAA IGAGGAGA | 5277 |
| 2965 | CCUCCAUC C UGGAGAAA | 1501 | UUUCUCCA CUGAUGAGGCCGUUAGGCCGAA IAUGGAGG | 5278 |
| 2966 | CUCCAUCC U GGAGAAAG | 1502 | CUUUCUCC CUGAUGAGGCCGUUAGGCCGAA IGAUGGAG | 5279 |
| 2983 | GAGAACGC C UCCCUCAG | 1503 | CUGAGGGA CUGAUGAGGCCGUUAGGCCGAA ICGUUCUC | 5280 |
| 2984 | AGAACGCC U CCCUCAGC | 1504 | GCUGAGGG CUGAUGAGGCCGUUAGGCCGAA IGCGUUCU | 5281 |
| 2986 | AACGCCUC C CUCAGCCA | 1505 | UGGCUGAG CUGAUGAGGCCGUUAGGCCGAA IAGGCGUU | 5282 |
| 2987 | ACGCCUCC C UCAGCCAC | 1506 | GUGGCUGA CUGAUGAGGCCGUUAGGCCGAA IGAGGCGU | 5283 |
| 2988 | CGCCUCCC U CAGCCACC | 1507 | GGUGGCUG CUGAUGAGGCCGUUAGGCCGAA IGGAGGCG | 5284 |
| 2990 | CCUCCCUC A GCCACCCA | 1508 | UGGGUGGC CUGAUGAGGCCGUUAGGCCGAA IAGGGAGG | 5285 |
| 2993 | CCCUCAGC C ACCCAUAU | 1509 | AUAUGGGU CUGAUGAGGCCGUUAGGCCGAA ICUGAGGG | 5286 |
| 2994 | CCUCAGCC A CCCAUAUG | 1510 | CAUAUGGG CUGAUGAGGCCGUUAGGCCGAA IGCUGAGG | 5287 |
| 2996 | UCAGCCAC C CAUAUGUA | 1511 | UACAUAUG CUGAUGAGGCCGUUAGGCCGAA IUGGCUGA | 5288 |
| 2997 | CAGCCACC C AUAUGUAC | 1512 | GUACAUAU CUGAUGAGGCCGUUAGGCCGAA IGUGGCUG | 5289 |
| 2998 | AGCCACCC A UAUGUACC | 1513 | GGUACAUA CUGAUGAGGCCGUUAGGCCGAA IGGUGGCU | 5290 |
| 3006 | AUAUGUAC C AUCGAUGU | 1514 | ACAUCGAU CUGAUGAGGCCGUUAGGCCGAA IUACAUAU | 5291 |
| 3007 | UAUGUACC A UCGAUGUC | 1515 | GACAUCGA CUGAUGAGGCCGUUAGGCCGAA IGUACAUA | 5292 |
| 3016 | UCGAUGUC U ACAUGAUC | 1516 | GAUCAUGU CUGAUGAGGCCGUUAGGCCGAA IACAUCGA | 5293 |
| 3019 | AUGUCUAC A UGAUCAUG | 1517 | CAUGAUCA CUGAUGAGGCCGUUAGGCCGAA IUAGACAU | 5294 |
| 3025 | ACAUGAUC A UGGUCAAG | 1518 | CUUGACCA CUGAUGAGGCCGUUAGGCCGAA IAUCAUGU | 5295 |
| 3031 | UCAUGGUC A AGUGCUGG | 1519 | CCAGCACU CUGAUGAGGCCGUUAGGCCGAA IACCAUGA | 5296 |
| 3037 | UCAAGUGC U GGAUGAUA | 1520 | UAUCAUCC CUGAUGAGGCCGUUAGGCCGAA ICACUUGA | 5297 |
| 3051 | AUAGACGC A GAUAGUCG | 1521 | CGACUAUC CUGAUGAGGCCGUUAGGCCGAA ICGUCUAU | 5298 |
| 3061 | AUAGUCGC C CAAAGUUC | 1522 | GAACUUUG CUGAUGAGGCCGUUAGGCCGAA ICGACUAU | 5299 |
| 3062 | UAGUCGCC C AAAGUUCC | 1523 | GGAACUUU CUGAUGAGGCCGUUAGGCCGAA IGCGACUA | 5300 |
| 3063 | AGUCGCCC A AAGUUCCG | 1524 | CGGAACUU CUGAUGAGGCCGUUAGGCCGAA IGGCGACU | 5301 |
| 3070 | CAAAGUUC C GUGAGUUG | 1525 | CAACUCAC CUGAUGAGGCCGUUAGGCCGAA IAACUUUG | 5302 |
| 3082 | AGUUGAUC A UCGAAUUC | 1526 | GAAUUCGA CUGAUGAGGCCGUUAGGCCGAA IAUCAACU | 5303 |
| 3091 | UCGAAUUC U CCAAAAUG | 1527 | CAUUUUGG CUGAUGAGGCCGUUAGGCCGAA IAAUUCGA | 5304 |
| 3093 | GAAUUCUC C AAAAUGGC | 1528 | GCCAUUUU CUGAUGAGGCCGUUAGGCCGAA IAGAAUUC | 5305 |
| 3094 | AAUUCUCC A AAAUGGCC | 1529 | GGCCAUUU CUGAUGAGGCCGUUAGGCCGAA IGAGAAUU | 5306 |
| 3102 | AAAAUGGC C CGAGACCC | 1530 | GGGUCUCG CUGAUGAGGCCGUUAGGCCGAA ICCAUUUU | 5307 |
| 3103 | AAAUGGCC C GAGACCCC | 1531 | GGGGUCUC CUGAUGAGGCCGUUAGGCCGAA IGCCAUUU | 5308 |
| 3109 | CCCGAGAC C CCCAGCGC | 1532 | GCGCUGGG CUGAUGAGGCCGUUAGGCCGAA IUCUCGGG | 5309 |
| 3110 | CCGAGACC C CCAGCGCU | 1533 | AGCGCUGG CUGAUGAGGCCGUUAGGCCGAA IGUCUCGG | 5310 |
| 3111 | CGAGACCC C CAGCGCUA | 1534 | UAGCGCUG CUGAUGAGGCCGUUAGGCCGAA IGGUCUCG | 5311 |
| 3112 | GAGACCCC C AGCGCUAC | 1535 | GUAGCGCU CUGAUGAGGCCGUUAGGCCGAA IGGGUCUC | 5312 |
| 3113 | AGACCCCC A GCGCUACC | 1536 | GGUAGCGC CUGAUGAGGCCGUUAGGCCGAA IGGGGUCU | 5313 |
| 3118 | CCCAGCGC U ACCUUGUC | 1537 | GACAAGGU CUGAUGAGGCCGUUAGGCCGAA ICGCUGGG | 5314 |
| 3121 | AGCGCUAC C UUGUCAUU | 1538 | AAUGACAA CUGAUGAGGCCGUUAGGCCGAA IUAGCGCU | 5315 |
| 3122 | GCGCUACC U UGUCAUUC | 1539 | GAAUGACA CUGAUGAGGCCGUUAGGCCGAA IGUAGCGC | 5316 |
| 3127 | ACCUUGUC A UUCAGGGG | 1540 | CCCCUGAA CUGAUGAGGCCGUUAGGCCGAA IACAAGGU | 5317 |
| 3131 | UGUCAUUC A GGGGGAUG | 1541 | CAUCCCCC CUGAUGAGGCCGUUAGGCCGAA IAAUGACA | 5318 |
| 3149 | AAGAAUGC A UUUGCCAA | 1542 | UUGGCAAA CUGAUGAGGCCGUUAGGCCGAA ICAUUCUU | 5319 |
| 3155 | GCAUUUGC C AAGUCCUA | 1543 | UAGGACUU CUGAUGAGGCCGUUAGGCCGAA ICAAAUGC | 5320 |
| 3156 | CAUUUGCC A AGUCCUAC | 1544 | GUAGGACU CUGAUGAGGCCGUUAGGCCGAA IGCAAAUG | 5321 |
| 3161 | GCCAAGUC C UACAGACU | 1545 | AGUCUGUA CUGAUGAGGCCGUUAGGCCGAA IACUUGGC | 5322 |
| 3162 | CCAAGUCC U ACAGACUC | 1546 | GAGUCUGU CUGAUGAGGCCGUUAGGCCGAA IGACUUGG | 5323 |
| 3165 | AGUCCUAC A GACUCCAA | 1547 | UUGGAGUC CUGAUGAGGCCGUUAGGCCGAA IUAGGACU | 5324 |
| 3169 | CUACAGAC U CCAACUUC | 1548 | GAAGUUGG CUGAUGAGGCCGUUAGGCCGAA IUCUGUAG | 5325 |
| 3171 | ACAGACUC C AACUUCUA | 1549 | UAGAAGUU CUGAUGAGGCCGUUAGGCCGAA IAGUCUGU | 5326 |
| 3172 | CAGACUCC A ACUUCUAC | 1550 | GUAGAAGU CUGAUGAGGCCGUUAGGCCGAA IGAGUCUG | 5327 |
| 3175 | ACUCCAAC U UCUACCGU | 1551 | ACGGUAGA CUGAUGAGGCCGUUAGGCCGAA IUUGGAGU | 5328 |
| 3178 | CCAACUUC U ACCGUGCC | 1552 | GGCACGGU CUGAUGAGGCCGUUAGGCCGAA IAAGUUGG | 5329 |
| 3181 | ACUUCUAC C GUGCCCUG | 1553 | CAGGGCAC CUGAUGAGGCCGUUAGGCCGAA IUAGAAGU | 5330 |
| 3186 | UACCGUGC C CUGAUGGA | 1554 | UCCAUCAG CUGAUGAGGCCGUUAGGCCGAA ICACGGUA | 5331 |
| 3187 | ACCGUGCC C UGAUGGAU | 1555 | AUCCAUCA CUGAUGAGGCCGUUAGGCCGAA IGCACGGU | 5332 |
| 3188 | CCGUGCCC U GAUGGAUG | 1556 | CAUCCAUC CUGAUGAGGCCGUUAGGCCGAA IGGCACGG | 5333 |
| 3205 | AAGAAGAC A UGGACGAC | 1557 | GUCGUCCA CUGAUGAGGCCGUUAGGCCGAA IUCUUCUU | 5334 |
| 3225 | GUGGAUGC C GACGAGUA | 1558 | UACUCGUC CUGAUGAGGCCGUUAGGCCGAA ICAUCCAC | 5335 |
| 3235 | ACGAGUAC C UCAUCCCA | 1559 | UGGGAUGA CUGAUGAGGCCGUUAGGCCGAA IUACUCGU | 5336 |
| 3236 | CGAGUACC U CAUCCCAC | 1560 | GUGGGAUG CUGAUGAGGCCGUUAGGCCGAA IGUACUCG | 5337 |
| 3238 | AGUACCUC A UCCCACAG | 1561 | CUGUGGGA CUGAUGAGGCCGUUAGGCCGAA IAGGUACU | 5338 |
| 3241 | ACCUCAUC C CACAGCAG | 1562 | CUGCUGUG CUGAUGAGGCCGUUAGGCCGAA IAUGAGGU | 5339 |
| 3242 | CCUCAUCC C ACAGCAGG | 1563 | CCUGCUGU CUGAUGAGGCCGUUAGGCCGAA IGAUGAGG | 5340 |
| 3243 | CUCAUCCC A CAGCAGGG | 1564 | CCCUGCUG CUGAUGAGGCCGUUAGGCCGAA IGGAUGAG | 5341 |
| 3245 | CAUCCCAC A GCAGGGCU | 1565 | AGCCCUGC CUGAUGAGGCCGUUAGGCCGAA IUGGGAUG | 5342 |
| 3248 | CCCACAGC A GGGCUUCU | 1566 | AGAAGCCC CUGAUGAGGCCGUUAGGCCGAA ICUGUGGG | 5343 |
| 3253 | AGCAGGGC U UCUUCAGC | 1567 | GCUGAAGA CUGAUGAGGCCGUUAGGCCGAA ICCCUGCU | 5344 |
| 3256 | AGGGCUUC U UCAGCAGC | 1568 | GCUGCUGA CUGAUGAGGCCGUUAGGCCGAA IAAGCCCU | 5345 |
| 3259 | GCUUCUUC A GCAGCCCC | 1569 | GGGGCUGC CUGAUGAGGCCGUUAGGCCGAA IAAGAAGC | 5346 |
| 3262 | UCUUCAGC A GCCCCUCC | 1570 | GGAGGGGC CUGAUGAGGCCGUUAGGCCGAA ICUGAAGA | 5347 |
| 3265 | UCAGCAGC C CCUCCACG | 1571 | CGUGGAGG CUGAUGAGGCCGUUAGGCCGAA ICUGCUGA | 5348 |
| 3266 | CAGCAGCC C CUCCACGU | 1572 | ACGUGGAG CUGAUGAGGCCGUUAGGCCGAA IGCUGCUG | 5349 |
| 3267 | AGCAGCCC C UCCACGUC | 1573 | GACGUGGA CUGAUGAGGCCGUUAGGCCGAA IGGCUGCU | 5350 |
| 3268 | GCAGCCCC U CCACGUCA | 1574 | UGACGUGG CUGAUGAGGCCGUUAGGCCGAA IGGGCUGC | 5351 |
| 3270 | AGCCCCUC C ACGUCACG | 1575 | CGUGACGU CUGAUGAGGCCGUUAGGCCGAA IAGGGGCU | 5352 |
| 3271 | GCCCCUCC A CGUCACGG | 1576 | CCGUGACG CUGAUGAGGCCGUUAGGCCGAA IGAGGGGC | 5353 |
| 3276 | UCCACGUC A CGGACUCC | 1577 | GGAGUCCG CUGAUGAGGCCGUUAGGCCGAA IACGUGGA | 5354 |
| 3282 | UCACGGAC U CCCCUCCU | 1578 | AGGAGGGG CUGAUGAGGCCGUUAGGCCGAA IUCCGUGA | 5355 |
| 3284 | ACGGACUC C CCUCCUGA | 1579 | UCAGGAGG CUGAUGAGGCCGUUAGGCCGAA IAGUCCGU | 5356 |
| 3285 | CGGACUCC C CUCCUGAG | 1580 | CUCAGGAG CUGAUGAGGCCGUUAGGCCGAA IGAGUCCG | 5357 |
| 3286 | GGACUCCC C UCCUGAGC | 1581 | GCUCAGGA CUGAUGAGGCCGUUAGGCCGAA IGGAGUCC | 5358 |
| 3287 | GACUCCCC U CCUGAGCU | 1582 | AGCUCAGG CUGAUGAGGCCGUUAGGCCGAA IGGGAGUC | 5359 |
| 3289 | CUCCCCUC C UGAGCUCU | 1583 | AGAGCUCA CUGAUGAGGCCGUUAGGCCGAA IAGGGGAG | 5360 |
| 3290 | UCCCCUCC U GAGCUCUC | 1584 | GAGAGCUC CUGAUGAGGCCGUUAGGCCGAA IGAGGGGA | 5361 |
| 3295 | UCCUGAGC U CUCUGAGU | 1585 | ACUCAGAG CUGAUGAGGCCGUUAGGCCGAA ICUCAGGA | 5362 |
| 3297 | CUGAGCUC U CUGAGUGC | 1586 | GCACUCAG CUGAUGAGGCCGUUAGGCCGAA IAGCUCAG | 5363 |
| 3299 | GAGCUCUC U GAGUGCAA | 1587 | UUGCACUC CUGAUGAGGCCGUUAGGCCGAA IAGAGCUC | 5364 |
| 3306 | CUGAGUGC A ACCAGCAA | 1588 | UUGCUGGU CUGAUGAGGCCGUUAGGCCGAA ICACUCAG | 5365 |
| 3309 | AGUGCAAC C AGCAACAA | 1589 | UUGUUGCU CUGAUGAGGCCGUUAGGCCGAA IUUGCACU | 5366 |
| 3310 | GUGCAACC A GCAACAAU | 1590 | AUUGUUGC CUGAUGAGGCCGUUAGGCCGAA IGUUGCAC | 5367 |
| 3313 | CAACCAGC A ACAAUUCC | 1591 | GGAAUUGU CUGAUGAGGCCGUUAGGCCGAA ICUGGUUG | 5368 |
| 3316 | CCAGCAAC A AUUCCACC | 1592 | GGUGGAAU CUGAUGAGGCCGUUAGGCCGAA IUUGCUGG | 5369 |
| 3321 | AACAAUUC C ACCGUGGC | 1593 | GCCACGGU CUGAUGAGGCCGUUAGGCCGAA IAAUUGUU | 5370 |
| 3322 | ACAAUUCC A CCGUGGCU | 1594 | AGCCACGG CUGAUGAGGCCGUUAGGCCGAA IGAAUUGU | 5371 |
| 3324 | AAUUCCAC C GUGGCUUG | 1595 | CAAGCCAC CUGAUGAGGCCGUUAGGCCGAA IUGGAAUU | 5372 |
| 3330 | ACCGUGGC U UGCAUUGA | 1596 | UCAAUGCA CUGAUGAGGCCGUUAGGCCGAA ICCACGGU | 5373 |
| 3334 | UGGCUUGC A UUGAUAGA | 1597 | UCUAUCAA CUGAUGAGGCCGUUAGGCCGAA ICAAGCCA | 5374 |
| 3350 | AAAUGGGC U GCAAAGCU | 1598 | AGCUUUGC CUGAUGAGGCCGUUAGGCCGAA ICCCAUUU | 5375 |
| 3353 | UGGGCUGC A AAGCUGUC | 1599 | GACAGCUU CUGAUGAGGCCGUUAGGCCGAA ICAGCCCA | 5376 |
| 3358 | UGCAAAGC U GUCCCAUC | 1600 | GAUGGGAC CUGAUGAGGCCGUUAGGCCGAA ICUUUGCA | 5377 |
| 3362 | AAGCUGUC C CAUCAAGG | 1601 | CCUUGAUG CUGAUGAGGCCGUUAGGCCGAA IACAGCUU | 5378 |
| 3363 | AGCUGUCC C AUCAAGGA | 1602 | UCCUUGAU CUGAUGAGGCCGUUAGGCCGAA IGACAGCU | 5379 |
| 3364 | GCUGUCCC A UCAAGGAA | 1603 | UUCCUUGA CUGAUGAGGCCGUUAGGCCGAA IGGACAGC | 5380 |
| 3367 | GUCCCAUC A AGGAAGAC | 1604 | GUCUUCCU CUGAUGAGGCCGUUAGGCCGAA IAUGGGAC | 5381 |
| 3376 | AGGAAGAC A GCUUCUUG | 1605 | CAAGAAGC CUGAUGAGGCCGUUAGGCCGAA IUCUUCCU | 5382 |
| 3379 | AAGACAGC U UCUUGCAG | 1606 | CUGCAAGA CUGAUGAGGCCGUUAGGCCGAA ICUGUCUU | 5383 |
| 3382 | ACAGCUUC U UGCAGCGA | 1607 | UCGCUGCA CUGAUGAGGCCGUUAGGCCGAA IAAGCUGU | 5384 |
| 3386 | CUUCUUGC A GCGAUACA | 1608 | UGUAUCGC CUGAUGAGGCCGUUAGGCCGAA ICAAGAAG | 5385 |
| 3394 | AGCGAUAC A GCUCAGAC | 1609 | GUCUGAGC CUGAUGAGGCCGUUAGGCCGAA IUAUCGCU | 5386 |
| 3397 | GAUACAGC U CAGACCCC | 1610 | GGGGUCUG CUGAUGAGGCCGUUAGGCCGAA ICUGUAUC | 5387 |
| 3399 | UACAGCUC A GACCCCAC | 1611 | GUGGGGUC CUGAUGAGGCCGUUAGGCCGAA IAGCUGUA | 5388 |
| 3403 | GCUCAGAC C CCACAGGC | 1612 | GCCUGUGG CUGAUGAGGCCGUUAGGCCGAA IUCUGAGC | 5389 |
| 3404 | CUCAGACC C CACAGGCG | 1613 | CGCCUGUG CUGAUGAGGCCGUUAGGCCGAA IGUCUGAG | 5390 |
| 3405 | UCAGACCC C ACAGGCGC | 1614 | GCGCCUGU CUGAUGAGGCCGUUAGGCCGAA IGGUCUGA | 5391 |
| 3406 | CAGACCCC A CAGGCGCC | 1615 | GGCGCCUG CUGAUGAGGCCGUUAGGCCGAA IGGGUCUG | 5392 |
| 3408 | GACCCCAC A GGCGCCUU | 1616 | AAGGCGCC CUGAUGAGGCCGUUAGGCCGAA IUGGGGUC | 5393 |
| 3414 | ACAGGCGC C UUGACUGA | 1617 | UCAGUCAA CUGAUGAGGCCGUUAGGCCGAA ICGCCUGU | 5394 |
| 3415 | CAGGCGCC U UGACUGAG | 1618 | CUCAGUCA CUGAUGAGGCCGUUAGGCCGAA IGCGCCUG | 5395 |
| 3420 | GCCUUGAC U GAGGACAG | 1619 | CUGUCCUC CUGAUGAGGCCGUUAGGCCGAA IUCAAGGC | 5396 |
| 3427 | CUGAGGAC A GCAUAGAC | 1620 | GUCUAUGC CUGAUGAGGCCGUUAGGCCGAA IUCCUCAG | 5397 |
| 3430 | AGGACAGC A UAGACGAC | 1621 | GUCGUCUA CUGAUGAGGCCGUUAGGCCGAA ICUGUCCU | 5398 |
| 3439 | UAGACGAC A CCUUCCUC | 1622 | GAGGAAGG CUGAUGAGGCCGUUAGGCCGAA IUCGUCUA | 5399 |
| 3441 | GACGACAC C UUCCUCCC | 1623 | GGGAGGAA CUGAUGAGGCCGUUAGGCCGAA IUGUCGUC | 5400 |
| 3442 | ACGACACC U UCCUCCCA | 1624 | UGGGAGGA CUGAUGAGGCCGUUAGGCCGAA IGUGUCGU | 5401 |
| 3445 | ACACCUUC C UCCCAGUG | 1625 | CACUGGGA CUGAUGAGGCCGUUAGGCCGAA IAAGGUGU | 5402 |
| 3446 | CACCUUCC U CCCAGUGC | 1626 | GCACUGGG CUGAUGAGGCCGUUAGGCCGAA IGAAGGUG | 5403 |
| 3448 | CCUUCCUC C CAGUGCCU | 1627 | AGGCACUG CUGAUGAGGCCGUUAGGCCGAA IAGGAAGG | 5404 |
| 3449 | CUUCCUCC C AGUGCCUG | 1628 | CAGGCACU CUGAUGAGGCCGUUAGGCCGAA IGAGGAAG | 5405 |
| 3450 | UUCCUCCC A GUGCCUGA | 1629 | UCAGGCAC CUGAUGAGGCCGUUAGGCCGAA IGGAGGAA | 5406 |
| 3455 | CCCAGUGC C UGAAUACA | 1630 | UGUAUUCA CUGAUGAGGCCGUUAGGCCGAA ICACUGGG | 5407 |
| 3456 | CCAGUGCC U GAAUACAU | 1631 | AUGUAUUC CUGAUGAGGCCGUUAGGCCGAA IGCACUGG | 5408 |
| 3463 | CUGAAUAC A UAAACCAG | 1632 | CUGGUUUA CUGAUGAGGCCGUUAGGCCGAA IUAUUCAG | 5409 |
| 3469 | ACAUAAAC C AGUCCGUU | 1633 | AACGGACU CUGAUGAGGCCGUUAGGCCGAA IUUUAUGU | 5410 |
| 3470 | CAUAAACC A GUCCGUUC | 1634 | GAACGGAC CUGAUGAGGCCGUUAGGCCGAA IGUUUAUG | 5411 |
| 3474 | AACCAGUC C GUUCCCAA | 1635 | UUGGGAAC CUGAUGAGGCCGUUAGGCCGAA IACUGGUU | 5412 |
| 3479 | GUCCGUUC C CAAAAGGC | 1636 | GCCUUUUG CUGAUGAGGCCGUUAGGCCGAA IAACGGAC | 5413 |
| 3480 | UCCGUUCC C AAAAGGCC | 1637 | GGCCUUUU CUGAUGAGGCCGUUAGGCCGAA IGAACGGA | 5414 |
| 3481 | CCGUUCCC A AAAGGCCC | 1638 | GGGCCUUU CUGAUGAGGCCGUUAGGCCGAA IGGAACGG | 5415 |
| 3488 | CAAAAGGC C CGCUGGCU | 1639 | AGCCAGCG CUGAUGAGGCCGUUAGGCCGAA ICCUUUUG | 5416 |
| 3489 | AAAAGGCC C GCUGGCUC | 1640 | GAGCCAGC CUGAUGAGGCCGUUAGGCCGAA IGCCUUUU | 5417 |
| 3492 | AGGCCCGC U GGCUCUGU | 1641 | ACAGAGCC CUGAUGAGGCCGUUAGGCCGAA ICGGGCCU | 5418 |
| 3496 | CCGCUGGC U CUGUGCAG | 1642 | CUGCACAG CUGAUGAGGCCGUUAGGCCGAA ICCAGCGG | 5419 |
| 3498 | GCUGGCUC U GUGCAGAA | 1643 | UUCUGCAC CUGAUGAGGCCGUUAGGCCGAA IAGCCAGC | 5420 |
| 3503 | CUCUGUGC A GAAUCCUG | 1644 | CAGGAUUC CUGAUGAGGCCGUUAGGCCGAA ICACAGAG | 5421 |
| 3509 | GCAGAAUC C UGUCUAUC | 1645 | GAUAGACA CUGAUGAGGCCGUUAGGCCGAA IAUUCUGC | 5422 |
| 3510 | CAGAAUCC U GUCUAUCA | 1646 | UGAUAGAC CUGAUGAGGCCGUUAGGCCGAA IGAUUCUG | 5423 |
| 3514 | AUCCUGUC U AUCACAAU | 1647 | AUUGUGAU CUGAUGAGGCCGUUAGGCCGAA IACAGGAU | 5424 |
| 3518 | UGUCUAUC A CAAUCAGC | 1648 | GCUGAUUG CUGAUGAGGCCGUUAGGCCGAA IAUAGACA | 5425 |
| 3520 | UCUAUCAC A AUCAGCCU | 1649 | AGGCUGAU CUGAUGAGGCCGUUAGGCCGAA IUGAUAGA | 5426 |
| 3524 | UCACAAUC A GCCUCUGA | 1650 | UCAGAGGC CUGAUGAGGCCGUUAGGCCGAA IAUUGUGA | 5427 |
| 3527 | CAAUCAGC C UCUGAACC | 1651 | GGUUCAGA CUGAUGAGGCCGUUAGGCCGAA ICUGAUUG | 5428 |
| 3528 | AAUCAGCC U CUGAACCC | 1652 | GGGUUCAG CUGAUGAGGCCGUUAGGCCGAA IGCUGAUU | 5429 |
| 3530 | UCAGCCUC U GAACCCCG | 1653 | CGGGGUUC CUGAUGAGGCCGUUAGGCCGAA IAGGCUGA | 5430 |
| 3535 | CUCUGAAC C CCGCGCCC | 1654 | GGGCGCGG CUGAUGAGGCCGUUAGGCCGAA IUUCAGAG | 5431 |
| 3536 | UCUGAACC C CGCGCCCA | 1655 | UGGGCGCG CUGAUGAGGCCGUUAGGCCGAA IGUUCAGA | 5432 |
| 3537 | CUGAACCC C GCGCCCAG | 1656 | CUGGGCGC CUGAUGAGGCCGUUAGGCCGAA IGGUUCAG | 5433 |
| 3542 | CCCCGCGC C CAGCAGAG | 1657 | CUCUGCUG CUGAUGAGGCCGUUAGGCCGAA ICGCGGGG | 5434 |
| 3543 | CCCGCGCC C AGCAGAGA | 1658 | UCUCUGCU CUGAUGAGGCCGUUAGGCCGAA IGCGCGGG | 5435 |
| 3544 | CCGCGCCC A GCAGAGAC | 1659 | GUCUCUGC CUGAUGAGGCCGUUAGGCCGAA IGGCGCGG | 5436 |
| 3547 | CGCCCAGC A GAGACCCA | 1660 | UGGGUCUC CUGAUGAGGCCGUUAGGCCGAA ICUGGGCG | 5437 |
| 3553 | GCAGAGAC C CACACUAC | 1661 | GUAGUGUG CUGAUGAGGCCGUUAGGCCGAA IUCUCUGC | 5438 |
| 3554 | CAGAGACC C ACACUACC | 1662 | GGUAGUGU CUGAUGAGGCCGUUAGGCCGAA IGUCUCUG | 5439 |
| 3555 | AGAGACCC A CACUACCA | 1663 | UGGUAGUG CUGAUGAGGCCGUUAGGCCGAA IGGUCUCU | 5440 |
| 3557 | AGACCCAC A CUACCAGG | 1664 | CCUGGUAG CUGAUGAGGCCGUUAGGCCGAA IUGGGUCU | 5441 |
| 3559 | ACCCACAC U ACCAGGAC | 1665 | GUCCUGGU CUGAUGAGGCCGUUAGGCCGAA IUGUGGGU | 5442 |
| 3562 | CACACUAC C AGGACCCC | 1666 | GGGGUCCU CUGAUGAGGCCGUUAGGCCGAA IUAGUGUG | 5443 |
| 3563 | ACACUACC A GGACCCCC | 1667 | GGGGGUCC CUGAUGAGGCCGUUAGGCCGAA IGUAGUGU | 5444 |
| 3568 | ACCAGGAC C CCCACAGC | 1668 | GCUGUGGG CUGAUGAGGCCGUUAGGCCGAA IUCCUGGU | 5445 |
| 3569 | CCAGGACC C CCACAGCA | 1669 | UGCUGUGG CUGAUGAGGCCGUUAGGCCGAA IGUCCUGG | 5446 |
| 3570 | CAGGACCC C CACAGCAC | 1670 | GUGCUGUG CUGAUGAGGCCGUUAGGCCGAA IGGUCCUG | 5447 |
| 3571 | AGGACCCC C ACAGCACU | 1671 | AGUGCUGU CUGAUGAGGCCGUUAGGCCGAA IGGGUCCU | 5448 |
| 3572 | GGACCCCC A CAGCACUG | 1672 | CAGUGCUG CUGAUGAGGCCGUUAGGCCGAA IGGGGUCC | 5449 |
| 3574 | ACCCCCAC A GCACUGCA | 1673 | UGCAGUGC CUGAUGAGGCCGUUAGGCCGAA IUGGGGGU | 5450 |
| 3577 | CCCACAGC A CUGCAGUG | 1674 | CACUGCAG CUGAUGAGGCCGUUAGGCCGAA ICUGUGGG | 5451 |
| 3579 | CACAGCAC U GCAGUGGG | 1675 | CCCACUGC CUGAUGAGGCCGUUAGGCCGAA IUGCUGUG | 5452 |
| 3582 | AGCACUGC A GUGGGCAA | 1676 | UUGCCCAC CUGAUGAGGCCGUUAGGCCGAA ICAGUGCU | 5453 |
| 3589 | CAGUGGGC A ACCCCGAG | 1677 | CUCGGGGU CUGAUGAGGCCGUUAGGCCGAA ICCCACUG | 5454 |
| 3592 | UGGGCAAC C CCGAGUAU | 1678 | AUACUCGG CUGAUGAGGCCGUUAGGCCGAA IUUGCCCA | 5455 |
| 3593 | GGGCAACC C CGAGUAUC | 1679 | GAUACUCG CUGAUGAGGCCGUUAGGCCGAA IGUUGCCC | 5456 |
| 3594 | GGCAACCC C GAGUAUCU | 1680 | AGAUACUC CUGAUGAGGCCGUUAGGCCGAA IGGUUGCC | 5457 |
| 3602 | CGAGUAUC U CAACACUG | 1681 | CAGUGUUG CUGAUGAGGCCGUUAGGCCGAA IAUACUCG | 5458 |
| 3604 | AGUAUCUC A ACACUGUC | 1682 | GACAGUGU CUGAUGAGGCCGUUAGGCCGAA IAGAUACU | 5459 |
| 3607 | AUCUCAAC A CUGUCCAG | 1683 | CUGGACAG CUGAUGAGGCCGUUAGGCCGAA IUUGAGAU | 5460 |
| 3609 | CUCAACAC U GUCCAGCC | 1684 | GGCUGGAC CUGAUGAGGCCGUUAGGCCGAA IUGUUGAG | 5461 |
| 3613 | ACACUGUC C AGCCCACC | 1685 | GGUGGGCU CUGAUGAGGCCGUUAGGCCGAA IACAGUGU | 5462 |
| 3614 | CACUGUCC A GCCCACCU | 1686 | AGGUGGGC CUGAUGAGGCCGUUAGGCCGAA IGACAGUG | 5463 |
| 3617 | UGUCCAGC C CACCUGUG | 1687 | CACAGGUG CUGAUGAGGCCGUUAGGCCGAA ICUGGACA | 5464 |
| 3618 | GUCCAGCC C ACCUGUGU | 1688 | ACACAGGU CUGAUGAGGCCGUUAGGCCGAA IGCUGGAC | 5465 |
| 3619 | UCCAGCCC A CCUGUGUC | 1689 | GACACAGG CUGAUGAGGCCGUUAGGCCGAA IGGCUGGA | 5466 |
| 3621 | CAGCCCAC C UGUGUCAA | 1690 | UUGACACA CUGAUGAGGCCGUUAGGCCGAA IUGGGCUG | 5467 |
| 3622 | AGCCCACC U GUGUCAAC | 1691 | GUUGACAC CUGAUGAGGCCGUUAGGCCGAA IGUGGGCU | 5468 |
| 3628 | CCUGUGUC A ACAGCACA | 1692 | UGUGCUGU CUGAUGAGGCCGUUAGGCCGAA IACACAGG | 5469 |
| 3631 | GUGUCAAC A GCACAUUC | 1693 | GAAUGUGC CUGAUGAGGCCGUUAGGCCGAA IUUGACAC | 5470 |
| 3634 | UCAACAGC A CAUUCGAC | 1694 | GUCGAAUG CUGAUGAGGCCGUUAGGCCGAA ICUGUUGA | 5471 |
| 3636 | AACAGCAC A UUCGACAG | 1695 | CUGUCGAA CUGAUGAGGCCGUUAGGCCGAA IUGCUGUU | 5472 |
| 3643 | CAUUCGAC A GCCCUGCC | 1696 | GGCAGGGC CUGAUGAGGCCGUUAGGCCGAA IUCGAAUG | 5473 |
| 3646 | UCGACAGC C CUGCCCAC | 1697 | GUGGGCAG CUGAUGAGGCCGUUAGGCCGAA ICUGUCGA | 5474 |
| 3647 | CGACAGCC C UGCCCACU | 1698 | AGUGGGCA CUGAUGAGGCCGUUAGGCCGAA IGCUGUCG | 5475 |
| 3648 | GACAGCCC U GCCCACUG | 1699 | CAGUGGGC CUGAUGAGGCCGUUAGGCCGAA IGGCUGUC | 5476 |
| 3651 | AGCCCUGC C CACUGGGC | 1700 | GCCCAGUG CUGAUGAGGCCGUUAGGCCGAA ICAGGGCU | 5477 |
| 3652 | GCCCUGCC C ACUGGGCC | 1701 | GGCCCAGU CUGAUGAGGCCGUUAGGCCGAA IGCAGGGC | 5478 |
| 3653 | CCCUGCCC A CUGGGCCC | 1702 | GGGCCCAG CUGAUGAGGCCGUUAGGCCGAA IGGCAGGG | 5479 |
| 3655 | CUGCCCAC U GGGCCCAG | 1703 | CUGGGCCC CUGAUGAGGCCGUUAGGCCGAA IUGGGCAG | 5480 |
| 3660 | CACUGGGC C CAGAAAGG | 1704 | CCUUUCUG CUGAUGAGGCCGUUAGGCCGAA ICCCAGUG | 5481 |
| 3661 | ACUGGGCC C AGAAAGGC | 1705 | GCCUUUCU CUGAUGAGGCCGUUAGGCCGAA IGCCCAGU | 5482 |
| 3662 | CUGGGCCC A GAAAGGCA | 1706 | UGCCUUUC CUGAUGAGGCCGUUAGGCCGAA IGGCCCAG | 5483 |
| 3670 | AGAAAGGC A GCCACCAA | 1707 | UUGGUGGC CUGAUGAGGCCGUUAGGCCGAA ICCUUUCU | 5484 |
| 3673 | AAGGCAGC C ACCAAAUU | 1708 | AAUUUGGU CUGAUGAGGCCGUUAGGCCGAA ICUGCCUU | 5485 |
| 3674 | AGGCAGCC A CCAAAUUA | 1709 | UAAUUUGG CUGAUGAGGCCGUUAGGCCGAA IGCUGCCU | 5486 |
| 3676 | GCAGCCAC C AAAUUAGC | 1710 | GCUAAUUU CUGAUGAGGCCGUUAGGCCGAA IUGGCUGC | 5487 |
| 3677 | CAGCCACC A AAUUAGCC | 1711 | GGCUAAUU CUGAUGAGGCCGUUAGGCCGAA IGUGGCUG | 5488 |
| 3685 | AAAUUAGC C UGGACAAC | 1712 | GUUGUCCA CUGAUGAGGCCGUUAGGCCGAA ICUAAUUU | 5489 |
| 3686 | AAUUAGCC U GGACAACC | 1713 | GGUUGUCC CUGAUGAGGCCGUUAGGCCGAA IGCUAAUU | 5490 |
| 3691 | GCCUGGAC A ACCCUGAC | 1714 | GUCAGGGU CUGAUGAGGCCGUUAGGCCGAA IUCCAGGC | 5491 |
| 3694 | UGGACAAC C CUGACUAC | 1715 | GUAGUCAG CUGAUGAGGCCGUUAGGCCGAA IUUGUCCA | 5492 |
| 3695 | GGACAACC C UGACUACC | 1716 | GGUAGUCA CUGAUGAGGCCGUUAGGCCGAA IGUUGUCC | 5493 |
| 3696 | GACAACCC U GACUACCA | 1717 | UGGUAGUC CUGAUGAGGCCGUUAGGCCGAA IGGUUGUC | 5494 |
| 3700 | ACCCUGAC U ACCAGCAG | 1718 | CUGCUGGU CUGAUGAGGCCGUUAGGCCGAA IUCAGGGU | 5495 |
| 3703 | CUGACUAC C AGCAGGAC | 1719 | GUCCUGCU CUGAUGAGGCCGUUAGGCCGAA IUAGUCAG | 5496 |
| 3704 | UGACUACC A GCAGGACU | 1720 | AGUCCUGC CUGAUGAGGCCGUUAGGCCGAA IGUAGUCA | 5497 |
| 3707 | CUACCAGC A GGACUUCU | 1721 | AGAAGUCC CUGAUGAGGCCGUUAGGCCGAA ICUGGUAG | 5498 |
| 3712 | AGCAGGAC U UCUUUCCC | 1722 | GGGAAAGA CUGAUGAGGCCGUUAGGCCGAA IUCCUGCU | 5499 |
| 3715 | AGGACUUC U UUCCCAAG | 1723 | CUUGGGAA CUGAUGAGGCCGUUAGGCCGAA IAAGUCCU | 5500 |
| 3719 | CUUCUUUC C CAAGGAAG | 1724 | CUUCCUUG CUGAUGAGGCCGUUAGGCCGAA IAAAGAAG | 5501 |
| 3720 | UUCUUUCC C AAGGAAGC | 1725 | GCUUCCUU CUGAUGAGGCCGUUAGGCCGAA IGAAAGAA | 5502 |
| 3721 | UCUUUCCC A AGGAAGCC | 1726 | GGCUUCCU CUGAUGAGGCCGUUAGGCCGAA IGGAAAGA | 5503 |
| 3729 | AAGGAAGC C AAGCCAAA | 1727 | UUUGGCUU CUGAUGAGGCCGUUAGGCCGAA ICUUCCUU | 5504 |
| 3730 | AGGAAGCC A AGCCAAAU | 1728 | AUUUGGCU CUGAUGAGGCCGUUAGGCCGAA IGCUUCCU | 5505 |
| 3734 | AGCCAAGC C AAAUGGCA | 1729 | UGCCAUUU CUGAUGAGGCCGUUAGGCCGAA ICUUGGCU | 5506 |
| 3735 | GCCAAGCC A AAUGGCAU | 1730 | AUGCCAUU CUGAUGAGGCCGUUAGGCCGAA IGCUUGGC | 5507 |
| 3742 | CAAAUGGC A UCUUUAAG | 1731 | CUUAAAGA CUGAUGAGGCCGUUAGGCCGAA ICCAUUUG | 5508 |
| 3745 | AUGGCAUC U UUAAGGGC | 1732 | GCCCUUAA CUGAUGAGGCCGUUAGGCCGAA IAUGCCAU | 5509 |
| 3754 | UUAAGGGC U CCACAGCU | 1733 | AGCUGUGG CUGAUGAGGCCGUUAGGCCGAA ICCCUUAA | 5510 |
| 3756 | AAGGGCUC C ACAGCUGA | 1734 | UCAGCUGU CUGAUGAGGCCGUUAGGCCGAA IAGCCCUU | 5511 |
| 3757 | AGGGCUCC A CAGCUGAA | 1735 | UUCAGCUG CUGAUGAGGCCGUUAGGCCGAA IGAGCCCU | 5512 |
| 3759 | GGCUCCAC A GCUGAAAA | 1736 | UUUUCAGC CUGAUGAGGCCGUUAGGCCGAA IUGGAGCC | 5513 |
| 3762 | UCCACAGC U GAAAAUGC | 1737 | GCAUUUUC CUGAUGAGGCCGUUAGGCCGAA ICUGUGGA | 5514 |
| 3771 | GAAAAUGC A GAAUACCU | 1738 | AGGUAUUC CUGAUGAGGCCGUUAGGCCGAA ICAUUUUC | 5515 |
| 3778 | CAGAAUAC C UAAGGGUC | 1739 | GACCCUUA CUGAUGAGGCCGUUAGGCCGAA IUAUUCUG | 5516 |
| 3779 | AGAAUACC U AAGGGUCG | 1740 | CGACCCUU CUGAUGAGGCCGUUAGGCCGAA IGUAUUCU | 5517 |
| 3791 | GGUCGCGC C ACAAAGCA | 1741 | UGCUUUGU CUGAUGAGGCCGUUAGGCCGAA ICGCGACC | 5518 |
| 3792 | GUCGCGCC A CAAAGCAG | 1742 | CUGCUUUG CUGAUGAGGCCGUUAGGCCGAA IGCGCGAC | 5519 |
| 3794 | CGCGCCAC A AAGCAGUG | 1743 | CACUGCUU CUGAUGAGGCCGUUAGGCCGAA IUGGCGCG | 5520 |
| 3799 | CACAAAGC A GUGAAUUU | 1744 | AAAUUCAC CUGAUGAGGCCGUUAGGCCGAA ICUUUGUG | 5521 |
| 3816 | AUUGGAGC A UGACCACG | 1745 | CGUGGUCA CUGAUGAGGCCGUUAGGCCGAA ICUCCAAU | 5522 |
| 3821 | AGCAUGAC C ACGGAGGA | 1746 | UCCUCCGU CUGAUGAGGCCGUUAGGCCGAA IUCAUGCU | 5523 |
| 3822 | GCAUGACC A CGGAGGAU | 1747 | AUCCUCCG CUGAUGAGGCCGUUAGGCCGAA IGUCAUGC | 5524 |
| 3840 | GUAUGAGC C CUAAAAAU | 1748 | AUUUUUAG CUGAUGAGGCCGUUAGGCCGAA ICUCAUAC | 5525 |
| 3841 | UAUGAGCC C UAAAAAUC | 1749 | GAUUUUUA CUGAUGAGGCCGUUAGGCCGAA IGCUCAUA | 5526 |
| 3842 | AUGAGCCC U AAAAAUCC | 1750 | GGAUUUUU CUGAUGAGGCCGUUAGGCCGAA IGGCUCAU | 5527 |
| 3850 | UAAAAAUC C AGACUCUU | 1751 | AAGAGUCU CUGAUGAGGCCGUUAGGCCGAA IAUUUUUA | 5528 |
| 3851 | AAAAAUCC A GACUCUUU | 1752 | AAAGAGUC CUGAUGAGGCCGUUAGGCCGAA IGAUUUUU | 5529 |
| 3855 | AUCCAGAC U CUUUCGAU | 1753 | AUCGAAAG CUGAUGAGGCCGUUAGGCCGAA IUCUGGAU | 5530 |
| 3857 | CCAGACUC U UUCGAUAC | 1754 | GUAUCGAA CUGAUGAGGCCGUUAGGCCGAA IAGUCUGG | 5531 |
| 3866 | UUCGAUAC C CAGGACCA | 1755 | UGGUCCUG CUGAUGAGGCCGUUAGGCCGAA IUAUCGAA | 5532 |
| 3867 | UCGAUACC C AGGACCAA | 1756 | UUGGUCCU CUGAUGAGGCCGUUAGGCCGAA IGUAUCGA | 5533 |
| 3868 | CGAUACCC A GGACCAAG | 1757 | CUUGGUCC CUGAUGAGGCCGUUAGGCCGAA IGGUAUCG | 5534 |
| 3873 | CCCAGGAC C AAGCCACA | 1758 | UGUGGCUU CUGAUGAGGCCGUUAGGCCGAA IUCCUGGG | 5535 |
| 3874 | CCAGGACC A AGCCACAG | 1759 | CUGUGGCU CUGAUGAGGCCGUUAGGCCGAA IGUCCUGG | 5536 |
| 3878 | GACCAAGC C ACAGCAGG | 1760 | CCUGCUGU CUGAUGAGGCCGUUAGGCCGAA ICUUGGUC | 5537 |
| 3879 | ACCAAGCC A CAGCAGGU | 1761 | ACCUGCUG CUGAUGAGGCCGUUAGGCCGAA IGCUUGGU | 5538 |
| 3881 | CAAGCCAC A GCAGGUCC | 1762 | GGACCUGC CUGAUGAGGCCGUUAGGCCGAA IUGGCUUG | 5539 |
| 3884 | GCCACAGC A GGUCCUCC | 1763 | GGAGGACC CUGAUGAGGCCGUUAGGCCGAA ICUGUGGC | 5540 |
| 3889 | AGCAGGUC C UCCAUCCC | 1764 | GGGAUGGA CUGAUGAGGCCGUUAGGCCGAA IACCUGCU | 5541 |
| 3890 | GCAGGUCC U CCAUCCCA | 1765 | UGGGAUGG CUGAUGAGGCCGUUAGGCCGAA IGACCUGC | 5542 |
| 3892 | AGGUCCUC C AUCCCAAC | 1766 | GUUGGGAU CUGAUGAGGCCGUUAGGCCGAA IAGGACCU | 5543 |
| 3893 | GGUCCUCC A UCCCAACA | 1767 | UGUUGGGA CUGAUGAGGCCGUUAGGCCGAA IGAGGACC | 5544 |
| 3896 | CCUCCAUC C CAACAGCC | 1768 | GGCUGUUG CUGAUGAGGCCGUUAGGCCGAA IAUGGAGG | 5545 |
| 3897 | CUCCAUCC C AACAGCCA | 1769 | UGGCUGUU CUGAUGAGGCCGUUAGGCCGAA IGAUGGAG | 5546 |
| 3898 | UCCAUCCC A ACAGCCAU | 1770 | AUGGCUGU CUGAUGAGGCCGUUAGGCCGAA IGGAUGGA | 5547 |
| 3901 | AUCCCAAC A GCCAUGCC | 1771 | GGCAUGGC CUGAUGAGGCCGUUAGGCCGAA IUUGGGAU | 5548 |
| 3904 | CCAACAGC C AUGCCCGC | 1772 | GCGGGCAU CUGAUGAGGCCGUUAGGCCGAA ICUGUUGG | 5549 |
| 3905 | CAACAGCC A UGCCCGCA | 1773 | UGCGGGCA CUGAUGAGGCCGUUAGGCCGAA IGCUGUUG | 5550 |
| 3909 | AGCCAUGC C CGCAUUAG | 1774 | CUAAUGCG CUGAUGAGGCCGUUAGGCCGAA ICAUGGCU | 5551 |
| 3910 | GCCAUGCC C GCAUUAGC | 1775 | GCUAAUGC CUGAUGAGGCCGUUAGGCCGAA IGCAUGGC | 5552 |
| 3913 | AUGCCCGC A UUAGCUCU | 1776 | AGAGCUAA CUGAUGAGGCCGUUAGGCCGAA ICGGGCAU | 5553 |
| 3919 | GCAUUAGC U CUUAGACC | 1777 | GGUCUAAG CUGAUGAGGCCGUUAGGCCGAA ICUAAUGC | 5554 |
| 3921 | AUUAGCUC U UAGACCCA | 1778 | UGGGUCUA CUGAUGAGGCCGUUAGGCCGAA IAGCUAAU | 5555 |
| 3927 | UCUUAGAC C CACAGACU | 1779 | AGUCUGUG CUGAUGAGGCCGUUAGGCCGAA IUCUAAGA | 5556 |
| 3928 | CUUAGACC C ACAGACUG | 1780 | CAGUCUGU CUGAUGAGGCCGUUAGGCCGAA IGUCUAAG | 5557 |
| 3929 | UUAGACCC A CAGACUGG | 1781 | CCAGUCUG CUGAUGAGGCCGUUAGGCCGAA IGGUCUAA | 5558 |
| 3931 | AGACCCAC A GACUGGUU | 1782 | AACCAGUC CUGAUGAGGCCGUUAGGCCGAA IUGGGUCU | 5559 |
| 3935 | CCACAGAC U GGUUUUGC | 1783 | GCAAAACC CUGAUGAGGCCGUUAGGCCGAA IUCUGUGG | 5560 |
| 3944 | GGUUUUGC A ACGUUUAC | 1784 | GUAAACGU CUGAUGAGGCCGUUAGGCCGAA ICAAAACC | 5561 |
| 3953 | ACGUUUAC A CCGACUAG | 1785 | CUAGUCGG CUGAUGAGGCCGUUAGGCCGAA IUAAACGU | 5562 |
| 3955 | GUUUACAC C GACUAGCC | 1786 | GGCUAGUC CUGAUGAGGCCGUUAGGCCGAA IUGUAAAC | 5563 |
| 3959 | ACACCGAC U AGCCAGGA | 1787 | UCCUGGCU CUGAUGAGGCCGUUAGGCCGAA IUCGGUGU | 5564 |
| 3963 | CGACUAGC C AGGAAGUA | 1788 | UACUUCCU CUGAUGAGGCCGUUAGGCCGAA ICUAGUCG | 5565 |
| 3964 | GACUAGCC A GGAAGUAC | 1789 | GUACUUCC CUGAUGAGGCCGUUAGGCCGAA IGCUAGUC | 5566 |
| 3973 | GGAAGUAC U UCCACCUC | 1790 | GAGGUGGA CUGAUGAGGCCGUUAGGCCGAA IUACUUCC | 5567 |
| 3976 | AGUACUUC C ACCUCGGG | 1792 | CCCGAGGU CUGAUGAGGCCGUUAGGCCGAA IAAGUACU | 5568 |
| 3977 | GUACUUCC A CCUCGGGC | 1792 | GCCCGAGG CUGAUGAGGCCGUUAGGCCGAA IGAAGUAC | 5569 |
| 3979 | ACUUCCAC C UCGGGCAC | 1793 | GUGCCCGA CUGAUGAGGCCGUUAGGCCGAA IUGGAAGU | 5570 |
| 3980 | CUUCCACC U CGGGCACA | 1794 | UGUGCCCG CUGAUGAGGCCGUUAGGCCGAA IGUGGAAG | 5571 |
| 3986 | CCUCGGGC A CAUUUUGG | 1795 | CCAAAAUG CUGAUGAGGCCGUUAGGCCGAA ICCCGAGG | 5572 |
| 3988 | UCGGGCAC A UUUUGGGA | 1796 | UCCCAAAA CUGAUGAGGCCGUUAGGCCGAA IUGCCCGA | 5573 |
| 4003 | GAAGUUGC A UUCCUUUG | 1797 | CAAAGGAA CUGAUGAGGCCGUUAGGCCGAA ICAACUUC | 5574 |
| 4007 | UUGCAUUC C UUUGUCUU | 1798 | AAGACAAA CUGAUGAGGCCGUUAGGCCGAA IAAUGCAA | 5575 |
| 4008 | UGCAUUCC U UUGUCUUC | 1799 | GAAGACAA CUGAUGAGGCCGUUAGGCCGAA IGAAUGCA | 5576 |
| 4014 | CCUUUGUC U UCAAACUG | 1800 | CAGUUUGA CUGAUGAGGCCGUUAGGCCGAA IACAAAGG | 5577 |
| 4017 | UUGUCUUC A AACUGUGA | 1801 | UCACAGUU CUGAUGAGGCCGUUAGGCCGAA IAAGACAA | 5578 |
| 4021 | CUUCAAAC U GUGAAGCA | 1802 | UGCUUCAC CUGAUGAGGCCGUUAGGCCGAA IUUUGAAG | 5579 |
| 4029 | UGUGAAGC A UUUACAGA | 1803 | UCUGUAAA CUGAUGAGGCCGUUAGGCCGAA ICUUCACA | 5580 |
| 4035 | GCAUUUAC A GAAACGCA | 1804 | UGCGUUUC CUGAUGAGGCCGUUAGGCCGAA IUAAAUGC | 5581 |
| 4043 | AGAAACGC A UCCAGCAA | 1805 | UUGCUGGA CUGAUGAGGCCGUUAGGCCGAA ICGUUUCU | 5582 |
| 4046 | AACGCAUC C AGCAAGAA | 1806 | UUCUUGCU CUGAUGAGGCCGUUAGGCCGAA IAUGCGUU | 5583 |
| 4047 | ACGCAUCC A GCAAGAAU | 1807 | AUUCUUGC CUGAUGAGGCCGUUAGGCCGAA IGAUGCGU | 5584 |
| 4050 | CAUCCAGC A AGAAUAUU | 1808 | AAUAUUCU CUGAUGAGGCCGUUAGGCCGAA ICUGGAUG | 5585 |
| 4062 | AUAUUGUC C CUUUGAGC | 1809 | GCUCAAAG CUGAUGAGGCCGUUAGGCCGAA IACAAUAU | 5586 |
| 4063 | UAUUGUCC C UUUGAGCA | 1810 | UGCUCAAA CUGAUGAGGCCGUUAGGCCGAA IGACAAUA | 5587 |
| 4064 | AUUGUCCC U UUGAGCAG | 1811 | CUGCUCAA CUGAUGAGGCCGUUAGGCCGAA IGGACAAU | 5588 |
| 4071 | CUUUGAGC A GAAAUUUA | 1812 | UAAAUUUC CUGAUGAGGCCGUUAGGCCGAA ICUCAAAG | 5589 |
| 4082 | AAUUUAUC U UUCAAAGA | 1813 | UCUUUGAA CUGAUGAGGCCGUUAGGCCGAA IAUAAAUU | 5590 |
| 4086 | UAUCUUUC A AAGAGGUA | 1814 | UACCUCUU CUGAUGAGGCCGUUAGGCCGAA IAAAGAUA | 5591 |
| 4148 | UGGGGAUC U UGGAGUUU | 1815 | AAACUCCA CUGAUGAGGCCGUUAGGCCGAA IAUCCCCA | 5592 |
| 4160 | AGUUUUUC A UUGUCGCU | 1816 | AGCGACAA CUGAUGAGGCCGUUAGGCCGAA IAAAAACU | 5593 |
| 4168 | AUUGUCGC U AUUGAUUU | 1817 | AAAUCAAU CUGAUGAGGCCGUUAGGCCGAA ICGACAAU | 5594 |
| 4181 | AUUUUUAC U UCAAUGGG | 1818 | CCCAUUGA CUGAUGAGGCCGUUAGGCCGAA IUAAAAAU | 5595 |
| 4184 | UUUACUUC A AUGGGCUC | 1819 | GAGCCCAU CUGAUGAGGCCGUUAGGCCGAA IAAGUAAA | 5596 |
| 4191 | CAAUGGGC U CUUCCAAC | 1820 | GUUGGAAG CUGAUGAGGCCGUUAGGCCGAA ICCCAUUG | 5597 |
| 4193 | AUGGGCUC U UCCAACAA | 1821 | UUGUUGGA CUGAUGAGGCCGUUAGGCCGAA IAGCCCAU | 5598 |
| 4196 | GGCUCUUC C AACAAGGA | 1822 | UCCUUGUU CUGAUGAGGCCGUUAGGCCGAA IAAGAGCC | 5599 |
| 4197 | GCUCUUCC A ACAAGGAA | 1823 | UUCCUUGU CUGAUGAGGCCGUUAGGCCGAA IGAAGAGC | 5600 |
| 4200 | CUUCCAAC A AGGAAGAA | 1824 | UUCUUCCU CUGAUGAGGCCGUUAGGCCGAA IUUGGAAG | 5601 |
| 4211 | GAAGAAGC U UGCUGGUA | 1825 | UACCAGCA CUGAUGAGGCCGUUAGGCCGAA ICUUCUUC | 5602 |
| 4215 | AAGCUUGC U GGUAGCAC | 1826 | GUGCUACC CUGAUGAGGCCGUUAGGCCGAA ICAAGCUU | 5603 |
| 4222 | CUGGUAGC A CUUGCUAC | 1827 | GUAGCAAG CUGAUGAGGCCGUUAGGCCGAA ICUACCAG | 5604 |
| 4224 | GGUAGCAC U UGCUACCC | 1828 | GGGUAGCA CUGAUGAGGCCGUUAGGCCGAA IUGCUACC | 5605 |
| 4228 | GCACUUGC U ACCCUGAG | 1829 | CUCAGGGU CUGAUGAGGCCGUUAGGCCGAA ICAAGUGC | 5606 |
| 4231 | CUUGCUAC C CUGAGUUC | 1830 | GAACUCAG CUGAUGAGGCCGUUAGGCCGAA IUAGCAAG | 5607 |
| 4232 | UUGCUACC C UGAGUUCA | 1831 | UGAACUCA CUGAUGAGGCCGUUAGGCCGAA IGUAGCAA | 5608 |
| 4233 | UGCUACCC U GAGUUCAU | 1832 | AUGAACUC CUGAUGAGGCCGUUAGGCCGAA IGGUAGCA | 5609 |
| 4240 | CUGAGUUC A UCCAGGCC | 1833 | GGCCUGGA CUGAUGAGGCCGUUAGGCCGAA IAACUCAG | 5610 |
| 4243 | AGUUCAUC C AGGCCCAA | 1834 | UUGGGCCU CUGAUGAGGCCGUUAGGCCGAA IAUGAACU | 5611 |
| 4244 | GUUCAUCC A GGCCCAAC | 1835 | GUUGGGCC CUGAUGAGGCCGUUAGGCCGAA IGAUGAAC | 5612 |
| 4248 | AUCCAGGC C CAACUGUG | 1836 | CACAGUUG CUGAUGAGGCCGUUAGGCCGAA ICCUGGAU | 5613 |
| 4249 | UCCAGGCC C AACUGUGA | 1837 | UCACAGUU CUGAUGAGGCCGUUAGGCCGAA IGCCUGGA | 5614 |
| 4250 | CCAGGCCC A ACUGUGAG | 1838 | CUCACAGU CUGAUGAGGCCGUUAGGCCGAA IGGCCUGG | 5615 |
| 4253 | GGCCCAAC U GUGAGCAA | 1839 | UUGCUCAC CUGAUGAGGCCGUUAGGCCGAA IUUGGGCC | 5616 |
| 4260 | CUGUGAGC A AGGAGCAC | 1840 | GUGCUCCU CUGAUGAGGCCGUUAGGCCGAA ICUCACAG | 5617 |
| 4267 | CAAGGAGC A CAAGCCAC | 1841 | GUGGCUUG CUGAUGAGGCCGUUAGGCCGAA ICUCCUUG | 5618 |
| 4269 | AGGAGCAC A AGCCACAA | 1842 | UUGUGGCU CUGAUGAGGCCGUUAGGCCGAA IUGCUCCU | 5619 |
| 4273 | GCACAAGC C ACAAGUCU | 1843 | AGACUUGU CUGAUGAGGCCGUUAGGCCGAA ICUUGUGC | 5620 |
| 4274 | CACAAGCC A CAAGUCUU | 1844 | AAGACUUG CUGAUGAGGCCGUUAGGCCGAA IGCUUGUG | 5621 |
| 4276 | CAAGCCAC A AGUCUUCC | 1845 | GGAAGACU CUGAUGAGGCCGUUAGGCCGAA IUGGCUUG | 5622 |
| 4281 | CACAAGUC U UCCAGAGG | 1846 | CCUCUGGA CUGAUGAGGCCGUUAGGCCGAA IACUUGUG | 5623 |
| 4284 | AAGUCUUC C AGAGGAUG | 1847 | CAUCCUCU CUGAUGAGGCCGUUAGGCCGAA IAAGACUU | 5624 |
| 4285 | AGUCUUCC A GAGGAUGC | 1848 | GCAUCCUC CUGAUGAGGCCGUUAGGCCGAA IGAAGACU | 5625 |
| 4294 | GAGGAUGC U UGAUUCCA | 1849 | UGGAAUCA CUGAUGAGGCCGUUAGGCCGAA ICAUCCUC | 5626 |
| 4301 | CUUGAUUC C AGUGGUUC | 1850 | GAACCACU CUGAUGAGGCCGUUAGGCCGAA IAAUCAAG | 5627 |
| 4302 | UUGAUUCC A GUGGUUCU | 1851 | AGAACCAC CUGAUGAGGCCGUUAGGCCGAA IGAAUCAA | 5628 |
| 4310 | AGUGGUUC U GCUUCAAG | 1852 | CUUGAAGC CUGAUGAGGCCGUUAGGCCGAA IAACCACU | 5629 |
| 4313 | GGUUCUGC U UCAAGGCU | 1853 | AGCCUUGA CUGAUGAGGCCGUUAGGCCGAA ICAGAACC | 5630 |
| 4316 | UCUGCUUC A AGGCUUCC | 1854 | GGAAGCCU CUGAUGAGGCCGUUAGGCCGAA IAAGCAGA | 5631 |
| 4321 | UUCAAGGC U UCCACUGC | 1855 | GCAGUGGA CUGAUGAGGCCGUUAGGCCGAA ICCUUGAA | 5632 |
| 4324 | AAGGCUUC C ACUGCAAA | 1856 | UUUGCAGU CUGAUGAGGCCGUUAGGCCGAA IAAGCCUU | 5633 |
| 4325 | AGGCUUCC A CUGCAAAA | 1857 | UUUUGCAG CUGAUGAGGCCGUUAGGCCGAA IGAAGCCU | 5634 |
| 4327 | GCUUCCAC U GCAAAACA | 1858 | UGUUUUGC CUGAUGAGGCCGUUAGGCCGAA IUGGAAGC | 5635 |
| 4330 | UCCACUGC A AAACACUA | 1859 | UAGUGUUU CUGAUGAGGCCGUUAGGCCGAA ICAGUGGA | 5636 |
| 4335 | UGCAAAAC A CUAAAGAU | 1860 | AUCUUUAG CUGAUGAGGCCGUUAGGCCGAA IUUUUGCA | 5637 |
| 4337 | CAAAACAC U AAAGAUCC | 1861 | GGAUCUUU CUGAUGAGGCCGUUAGGCCGAA IUGUUUUG | 5638 |
| 4345 | UAAAGAUC C AAGAAGGC | 1862 | GCCUUCUU CUGAUGAGGCCGUUAGGCCGAA IAUCUUUA | 5639 |
| 4346 | AAAGAUCC A AGAAGGCC | 1863 | GGCCUUCU CUGAUGAGGCCGUUAGGCCGAA IGAUCUUU | 5640 |
| 4354 | AAGAAGGC C UUCAUGGC | 1864 | GCCAUGAA CUGAUGAGGCCGUUAGGCCGAA ICCUUCUU | 5641 |
| 4355 | AGAAGGCC U UCAUGGCC | 1865 | GGCCAUGA CUGAUGAGGCCGUUAGGCCGAA IGCCUUCU | 5642 |
| 4358 | AGGCCUUC A UGGCCCCA | 1866 | UGGGGCCA CUGAUGAGGCCGUUAGGCCGAA IAAGGCCU | 5643 |
| 4363 | UUCAUGGC C CCAGCAGG | 1867 | CCUGCUGG CUGAUGAGGCCGUUAGGCCGAA ICCAUGAA | 5644 |
| 4364 | UCAUGGCC C CAGCAGGC | 1868 | GCCUGCUG CUGAUGAGGCCGUUAGGCCGAA IGCCAUGA | 5645 |
| 4365 | CAUGGCCC C AGCAGGCC | 1869 | GGCCUGCU CUGAUGAGGCCGUUAGGCCGAA IGGCCAUG | 5646 |
| 4366 | AUGGCCCC A GCAGGCCG | 1870 | CGGCCUGC CUGAUGAGGCCGUUAGGCCGAA IGGGCCAU | 5647 |
| 4369 | GCCCCAGC A GGCCGGAU | 1871 | AUCCGGCC CUGAUGAGGCCGUUAGGCCGAA ICUGGGGC | 5648 |
| 4373 | CAGCAGGC C GGAUCGGU | 1872 | ACCGAUCC CUGAUGAGGCCGUUAGGCCGAA ICCUGCUG | 5649 |
| 4384 | AUCGGUAC U GUAUCAAG | 1873 | CUUGAUAC CUGAUGAGGCCGUUAGGCCGAA IUACCGAU | 5650 |
| 4390 | ACUGUAUC A AGUCAUGG | 1874 | CCAUGACU CUGAUGAGGCCGUUAGGCCGAA IAUACAGU | 5651 |
| 4395 | AUCAAGUC A UGGCAGGU | 1875 | ACCUGCCA CUGAUGAGGCCGUUAGGCCGAA IACUUGAU | 5652 |
| 4400 | GUCAUGGC A GGUACAGU | 1876 | ACUGUACC CUGAUGAGGCCGUUAGGCCGAA ICCAUGAC | 5653 |
| 4406 | GCAGGUAC A GUAGGAUA | 1877 | UAUCCUAC CUGAUGAGGCCGUUAGGCCGAA IUACCUGC | 5654 |
| 4418 | GGAUAAGC C ACUCUGUC | 1878 | GACAGAGU CUGAUGAGGCCGUUAGGCCGAA ICUUAUCC | 5655 |
| 4419 | GAUAAGCC A CUCUGUCC | 1879 | GGACAGAG CUGAUGAGGCCGUUAGGCCGAA IGCUUAUC | 5656 |
| 4421 | UAAGCCAC U CUGUCCCU | 1880 | AGGGACAG CUGAUGAGGCCGUUAGGCCGAA IUGGCUUA | 5657 |
| 4423 | AGCCACUC U GUCCCUUC | 1881 | GAAGGGAC CUGAUGAGGCCGUUAGGCCGAA IAGUGGCU | 5658 |
| 4427 | ACUCUGUC C CUUCCUGG | 1882 | CCAGGAAG CUGAUGAGGCCGUUAGGCCGAA IACAGAGU | 5659 |
| 4428 | CUCUGUCC C UUCCUGGG | 1883 | CCCAGGAA CUGAUGAGGCCGUUAGGCCGAA IGACAGAG | 5660 |
| 4429 | UCUGUCCC U UCCUGGGC | 1884 | GCCCAGGA CUGAUGAGGCCGUUAGGCCGAA IGGACAGA | 5661 |
| 4432 | GUCCCUUC C UGGGCAAA | 1885 | UUUGCCCA CUGAUGAGGCCGUUAGGCCGAA IAAGGGAC | 5662 |
| 4433 | UCCCUUCC U GGGCAAAG | 1886 | CUUUGCCC CUGAUGAGGCCGUUAGGCCGAA IGAAGGGA | 5663 |
| 4438 | UCCUGGGC A AAGAAGAA | 1887 | UUCUUCUU CUGAUGAGGCCGUUAGGCCGAA ICCCAGGA | 5664 |
| 4464 | AUGAAUUC U UCCUUAGA | 1888 | UCUAAGGA CUGAUGAGGCCGUUAGGCCGAA IAAUUCAU | 5665 |
| 4467 | AAUUCUUC C UUAGACUU | 1889 | AAGUCUAA CUGAUGAGGCCGUUAGGCCGAA IAAGAAUU | 5666 |
| 4468 | AUUCUUCC U UAGACUUA | 1890 | UAAGUCUA CUGAUGAGGCCGUUAGGCCGAA IGAAGAAU | 5667 |
| 4474 | CCUUAGAC U UACUUUUG | 1891 | CAAAAGUA CUGAUGAGGCCGUUAGGCCGAA IUCUAAGG | 5668 |
| 4478 | AGACUUAC U UUUGUAAA | 1892 | UUUACAAA CUGAUGAGGCCGUUAGGCCGAA IUAAGUCU | 5669 |
| 4493 | AAAAUGUC C CCACGGUA | 1893 | UACCGUGG CUGAUGAGGCCGUUAGGCCGAA IACAUUUU | 5670 |
| 4494 | AAAUGUCC C CACGGUAC | 1894 | GUACCGUG CUGAUGAGGCCGUUAGGCCGAA IGACAUUU | 5671 |
| 4495 | AAUGUCCC C ACGGUACU | 1895 | AGUACCGU CUGAUGAGGCCGUUAGGCCGAA IGGACAUU | 5672 |
| 4496 | AUGUCCCC A CGGUACUU | 1896 | AAGUACCG CUGAUGAGGCCGUUAGGCCGAA IGGGACAU | 5673 |
| 4503 | CACGGUAC U UACUCCCC | 1897 | GGGGAGUA CUGAUGAGGCCGUUAGGCCGAA IUACCGUG | 5674 |
| 4507 | GUACUUAC U CCCCACUG | 1898 | CAGUGGGG CUGAUGAGGCCGUUAGGCCGAA IUAAGUAC | 5675 |
| 4509 | ACUUACUC C CCACUGAU | 1899 | AUCAGUGG CUGAUGAGGCCGUUAGGCCGAA IAGUAAGU | 5676 |
| 4510 | CUUACUCC C CACUGAUG | 1900 | CAUCAGUG CUGAUGAGGCCGUUAGGCCGAA IGAGUAAG | 5677 |
| 4511 | UUACUCCC C ACUGAUGG | 1901 | CCAUCAGU CUGAUGAGGCCGUUAGGCCGAA IGGAGUAA | 5678 |
| 4512 | UACUCCCC A CUGAUGGA | 1902 | UCCAUCAG CUGAUGAGGCCGUUAGGCCGAA IGGGAGUA | 5679 |
| 4514 | CUCCCCAC U GAUGGACC | 1903 | GGUCCAUC CUGAUGAGGCCGUUAGGCCGAA IUGGGGAG | 5680 |
| 4522 | UGAUGGAC C AGUGGUUU | 1904 | AAACCACU CUGAUGAGGCCGUUAGGCCGAA IUCCAUCA | 5681 |
| 4523 | GAUGGACC A GUGGUUUC | 1905 | GAAACCAC CUGAUGAGGCCGUUAGGCCGAA IGUCCAUC | 5682 |
| 4532 | GUGGUUUC C AGUCAUGA | 1906 | UCAUGACU CUGAUGAGGCCGUUAGGCCGAA IAAACCAC | 5683 |
| 4533 | UGGUUUCC A GUCAUGAG | 1907 | CUCAUGAC CUGAUGAGGCCGUUAGGCCGAA IGAAACCA | 5684 |
| 4537 | UUCCAGUC A UGAGCGUU | 1908 | AACGCUCA CUGAUGAGGCCGUUAGGCCGAA IACUGGAA | 5685 |
| 4550 | CGUUAGAC U GACUUGUU | 1909 | AACAAGUC CUGAUGAGGCCGUUAGGCCGAA IUCUAACG | 5686 |
| 4554 | AGACUGAC U UGUUUGUC | 1910 | GACAAACA CUGAUGAGGCCGUUAGGCCGAA IUCAGUCU | 5687 |
| 4563 | UGUUUGUC U UCCAUUCC | 1911 | GGAAUGGA CUGAUGAGGCCGUUAGGCCGAA IACAAACA | 5688 |
| 4566 | UUGUCUUC C AUUCCAUU | 1912 | AAUGGAAU CUGAUGAGGCCGUUAGGCCGAA IAAGACAA | 5689 |
| 4567 | UGUCUUCC A UUCCAUUG | 1913 | CAAUGGAA CUGAUGAGGCCGUUAGGCCGAA IGAAGACA | 5690 |
| 4571 | UUCCAUUC C AUUGUUUU | 1914 | AAAACAAU CUGAUGAGGCCGUUAGGCCGAA IAAUGGAA | 5691 |
| 4572 | UCCAUUCC A UUGUUUUG | 1915 | CAAAACAA CUGAUGAGGCCGUUAGGCCGAA IGAAUGGA | 5692 |
| 4585 | UUUGAAAC U CAGUAUGC | 1916 | GCAUACUG CUGAUGAGGCCGUUAGGCCGAA IUUUCAAA | 5693 |
| 4587 | UGAAACUC A GUAUGCCG | 1917 | CGGCAUAC CUGAUGAGGCCGUUAGGCCGAA IAGUUUCA | 5694 |
| 4594 | CAGUAUGC C GCCCCUGU | 1918 | ACAGGGGC CUGAUGAGGCCGUUAGGCCGAA ICAUACUG | 5695 |
| 4597 | UAUGCCGC C CCUGUCUU | 1919 | AAGACAGG CUGAUGAGGCCGUUAGGCCGAA ICGGCAUA | 5696 |
| 4598 | AUGCCGCC C CUGUCUUG | 1920 | CAAGACAG CUGAUGAGGCCGUUAGGCCGAA IGCGGCAU | 5697 |
| 4599 | UGCCGCCC C UGUCUUGC | 1921 | GCAAGACA CUGAUGAGGCCGUUAGGCCGAA IGGCGGCA | 5698 |
| 4600 | GCCGCCCC U GUCUUGCU | 1922 | AGCAAGAC CUGAUGAGGCCGUUAGGCCGAA IGGGCGGC | 5699 |
| 4604 | CCCCUGUC U UGCUGUCA | 1923 | UGACAGCA CUGAUGAGGCCGUUAGGCCGAA IACAGGGG | 5700 |
| 4608 | UGUCUUGC U GUCAUGAA | 1924 | UUCAUGAC CUGAUGAGGCCGUUAGGCCGAA ICAAGACA | 5701 |
| 4612 | UUGCUGUC A UGAAAUCA | 1925 | UGAUUUCA CUGAUGAGGCCGUUAGGCCGAA IACAGCAA | 5702 |
| 4620 | AUGAAAUC A GCAAGAGA | 1926 | UCUCUUGC CUGAUGAGGCCGUUAGGCCGAA IAUUUCAU | 5703 |
| 4623 | AAAUCAGC A AGAGAGGA | 1927 | UCCUCUCU CUGAUGAGGCCGUUAGGCCGAA ICUGAUUU | 5704 |
| 4636 | AGGAUGAC A CAUCAAAU | 1928 | AUUUGAUG CUGAUGAGGCCGUUAGGCCGAA IUCAUCCU | 5705 |
| 4638 | GAUGACAC A UCAAAUAA | 1929 | UUAUUUGA CUGAUGAGGCCGUUAGGCCGAA IUGUCAUC | 5706 |
| 4641 | GACACAUC A AAUAAUAA | 1930 | UUAUUAUU CUGAUGAGGCCGUUAGGCCGAA IAUGUGUC | 5707 |
| 4651 | AUAAUAAC U CGGAUUCC | 1931 | GGAAUCCG CUGAUGAGGCCGUUAGGCCGAA IUUAUUAU | 5708 |
| 4659 | UCGGAUUC C AGCCCACA | 1932 | UGUGGGCU CUGAUGAGGCCGUUAGGCCGAA IAAUCCGA | 5709 |
| 4660 | CGGAUUCC A GCCCACAU | 1933 | AUGUGGGC CUGAUGAGGCCGUUAGGCCGAA IGAAUCCG | 5710 |
| 4663 | AUUCCAGC C CACAUUGG | 1934 | CCAAUGUG CUGAUGAGGCCGUUAGGCCGAA ICUGGAAU | 5711 |
| 4664 | UUCCAGCC C ACAUUGGA | 1935 | UCCAAUGU CUGAUGAGGCCGUUAGGCCGAA IGCUGGAA | 5712 |
| 4665 | UCCAGCCC A CAUUGGAU | 1936 | AUCCAAUG CUGAUGAGGCCGUUAGGCCGAA IGGCUGGA | 5713 |
| 4667 | CAGCCCAC A UUGGAUUC | 1937 | GAAUCCAA CUGAUGAGGCCGUUAGGCCGAA IUGGGCUG | 5714 |
| 4676 | UUGGAUUC A UCAGCAUU | 1938 | AAUGCUGA CUGAUGAGGCCGUUAGGCCGAA IAAUCCAA | 5715 |
| 4679 | GAUUCAUC A GCAUUUGG | 1939 | CCAAAUGC CUGAUGAGGCCGUUAGGCCGAA IAUGAAUC | 5716 |
| 4682 | UCAUCAGC A UUUGGACC | 1940 | GGUCCAAA CUGAUGAGGCCGUUAGGCCGAA ICUGAUGA | 5717 |
| 4690 | AUUUGGAC C AAUAGCCC | 1941 | GGGCUAUU CUGAUGAGGCCGUUAGGCCGAA IUCCAAAU | 5718 |
| 4691 | UUUGGACC A AUAGCCCA | 1942 | UGGGCUAU CUGAUGAGGCCGUUAGGCCGAA IGUCCAAA | 5719 |
| 4697 | CCAAUAGC C CACAGCUG | 1943 | CACCUGUG CUGAUGAGGCCGUUAGGCCGAA ICUAUUGG | 5720 |
| 4698 | CAAUAGCC C ACAGCUGA | 1944 | UCAGCUGU CUGAUGAGGCCGUUAGGCCGAA IGCUAUUG | 5721 |
| 4699 | AAUAGCCC A CAGCUGAG | 1945 | CUCAGCUG CUGAUGAGGCCGUUAGGCCGAA IGGCUAUU | 5722 |
| 4701 | UAGCCCAC A GCUGAGAA | 1946 | UUCUCAGC CUGAUGAGGCCGUUAGGCCGAA IUGGGCUA | 5723 |
| 4704 | CCCACAGC U GAGAAUGU | 1947 | ACAUUCUC CUGAUGAGGCCGUUAGGCCGAA ICUGUGGG | 5724 |
| 4720 | UGGAAUAC C UAAGGAUA | 1948 | UAUCCUUA CUGAUGAGGCCGUUAGGCCGAA IUAUUCCA | 5725 |
| 4721 | GGAAUACC U AAGGAUAA | 1949 | UUAUCCUU CUGAUGAGGCCGUUAGGCCGAA IGUAUUCC | 5726 |
| 4731 | AGGAUAAC A CCGCUUUU | 1950 | AAAAGCGG CUGAUGAGGCCGUUAGGCCGAA IUUAUCCU | 5727 |
| 4733 | GAUAACAC C GCUUUUGU | 1951 | ACAAAAGC CUGAUGAGGCCGUUAGGCCGAA IUGUUAUC | 5728 |
| 4736 | AACACCGC U UUUGUUCU | 1952 | AGAACAAA CUGAUGAGGCCGUUAGGCCGAA ICGGUGUU | 5729 |
| 4744 | UUUUGUUC U CGCAAAAA | 1953 | UUUUUGCG CUGAUGAGGCCGUUAGGCCGAA IAACAAAA | 5730 |
| 4748 | GUUCUCGC A AAAACGUA | 1954 | UACGUUUU CUGAUGAGGCCGUUAGGCCGAA ICGAGAAC | 5731 |
| 4759 | AACGUAUC U CCUAAUUU | 1955 | AAAUUAGG CUGAUGAGGCCGUUAGGCCGAA IAUACGUU | 5732 |
| 4761 | CGUAUCUC C UAAUUUGA | 1956 | UCAAAUUA CUGAUGAGGCCGUUAGGCCGAA IAGAUACG | 5733 |
| 4762 | GUAUCUCC U AAUUUGAG | 1957 | CUCAAAUU CUGAUGAGGCCGUUAGGCCGAA IGAGAUAC | 5734 |
| 4773 | UUUGAGGC U CAGAUGAA | 1958 | UUCAUCUG CUGAUGAGGCCGUUAGGCCGAA ICCUCAAA | 5735 |
| 4775 | UGAGGCUC A GAUGAAAU | 1959 | AUUUCAUC CUGAUGAGGCCGUUAGGCCGAA IAGCCUCA | 5736 |
| 4786 | UGAAAUGC A UCAGGUCC | 1960 | GGACCUGA CUGAUGAGGCCGUUAGGCCGAA ICAUUUCA | 5737 |
| 4789 | AAUGCAUC A GGUCCUUU | 1961 | AAAGGACC CUGAUGAGGCCGUUAGGCCGAA IAUGCAUU | 5738 |
| 4794 | AUCAGGUC C UUUGGGGC | 1962 | GCCCCAAA CUGAUGAGGCCGUUAGGCCGAA IACCUGAU | 5739 |
| 4795 | UCAGGUCC U UUGGGGCA | 1963 | UGCCCCAA CUGAUGAGGCCGUUAGGCCGAA IGACCUGA | 5740 |
| 4803 | UUUGGGGC A UAGAUCAG | 1964 | CUGAUCUA CUGAUGAGGCCGUUAGGCCGAA ICCCCAAA | 5741 |
| 4810 | CAUAGAUC A GAAGACUA | 1965 | UAGUCUUC CUGAUGAGGCCGUUAGGCCGAA IAUCUAUG | 5742 |
| 4817 | CAGAAGAC U ACAAAAAU | 1966 | AUUUUUGU CUGAUGAGGCCGUUAGGCCGAA IUCUUCUG | 5743 |
| 4820 | AAGACUAC A AAAAUGAA | 1967 | UUCAUUUU CUGAUGAGGCCGUUAGGCCGAA IUAGUCUU | 5744 |
| 4831 | AAUGAAGC U GCUCUGAA | 1968 | UUCAGAGC CUGAUGAGGCCGUUAGGCCGAA ICUUCAUU | 5745 |
| 4834 | GAAGCUGC U CUGAAAUC | 1969 | GAUUUCAG CUGAUGAGGCCGUUAGGCCGAA ICAGCUUC | 5746 |
| 4836 | AGCUGCUC U GAAAUCUC | 1970 | GAGAUUUC CUGAUGAGGCCGUUAGGCCGAA IAGCAGCU | 5747 |
| 4843 | CUGAAAUC U CCUUUAGC | 1971 | GCUAAAGG CUGAUGAGGCCGUUAGGCCGAA IAUUUCAG | 5748 |
| 4845 | GAADUCUC C UUUAGCCA | 1972 | UGGCUAAA CUGAUGAGGCCGUUAGGCCGAA IAGAUUUC | 5749 |
| 4846 | AAAUCUCC U UUAGCCAU | 1973 | AUGGCUAA CUGAUGAGGCCGUUAGGCCGAA IGAGAUUU | 5750 |
| 4852 | CCUUUAGC C AUCACCCC | 1974 | GGGGUGAU CUGAUGAGGCCGUUAGGCCGAA ICUAAAGG | 5751 |
| 4853 | CUUUAGCC A UCACCCCA | 1975 | UGGGGUGA CUGAUGAGGCCGUUAGGCCGAA IGCUAAAG | 5752 |
| 4856 | UAGCCAUC A CCCCAACC | 1976 | GGUUGGGG CUGAUGAGGCCGUUAGGCCGAA IAUGGCUA | 5753 |
| 4858 | GCCAUCAC C CCAACCCC | 1977 | GGGGUUGG CUGAUGAGGCCGUUAGGCCGAA IUGAUGGC | 5754 |
| 4859 | CCAUCACC C CAACCCCC | 1978 | GGGGGUUG CUGAUGAGGCCGUUAGGCCGAA IGUGAUGG | 5755 |
| 4860 | CAUCACCC C AACCCCCC | 1979 | GGGGGGUU CUGAUGAGGCCGUUAGGCCGAA IGGUGAUG | 5756 |
| 4861 | AUCACCCC A ACCCCCCA | 1980 | UGGGGGGU CUGAUGAGGCCGUUAGGCCGAA IGGGUGAU | 5757 |
| 4864 | ACCCCAAC C CCCCAAAA | 1981 | UUUUGGGG CUGAUGAGGCCGUUAGGCCGAA IUUGGGGU | 5758 |
| 4865 | CCCCAACC C CCCAAAAU | 1982 | AUUUUGGG CUGAUGAGGCCGUUAGGCCGAA IGUUGGGG | 5759 |
| 4866 | CCCAACCC C CCAAAAUU | 1983 | AAUUUUGG CUGAUGAGGCCGUUAGGCCGAA IGGUUGGG | 5760 |
| 4867 | CCAACCCC C CAAAAUUA | 1984 | UAAUUUUG CUGAUGAGGCCGUUAGGCCGAA IGGGUUGG | 5761 |
| 4868 | CAACCCCC C AAAAUUAG | 1985 | CUAAUUUU CUGAUGAGGCCGUUAGGCCGAA IGGGGUUG | 5762 |
| 4869 | AACCCCCC A AAAUUAGU | 1986 | ACUAAUUU CUGAUGAGGCCGUUAGGCCGAA IGGGGGUU | 5763 |
| 4887 | UGUGUUAC U UAUGGAAG | 1987 | CUUCCAUA CUGAUGAGGCCGUUAGGCCGAA IUAACACA | 5764 |
| 4905 | UAGUUUUC U CCUUUUAC | 1988 | GUAAAAGG CUGAUGAGGCCGUUAGGCCGAA IAAAACUA | 5765 |
| 4907 | GUUUUCUC C UUUUACUU | 1989 | AAGUAAAA CUGAUGAGGCCGUUAGGCCGAA IAGAAAAC | 5766 |
| 4908 | UUUUCUCC U UUUACUUC | 1990 | GAAGUAAA CUGAUGAGGCCGUUAGGCCGAA IGAGAAAA | 5767 |
| 4914 | CCUUUUAC U UCACUUCA | 1991 | UGAAGUGA CUGAUGAGGCCGUUAGGCCGAA IUAAAAGG | 5768 |
| 4917 | UUUACUUC A CUUCAAAA | 1992 | UUUUGAAG CUGAUGAGGCCGUUAGGCCGAA IAAGUAAA | 5769 |
| 4919 | UACUUCAC U UCAAAAGC | 1993 | GCUUUUGA CUGAUGAGGCCGUUAGGCCGAA IUGAAGUA | 5770 |
| 4922 | UUCACUUC A AAAGCUUU | 1994 | AAAGCUUU CUGAUGAGGCCGUUAGGCCGAA IAAGUGAA | 5771 |
| 4928 | UCAAAAGC U UUUUACUC | 1995 | GAGUAAAA CUGAUGAGGCCGUUAGGCCGAA ICUUUUGA | 5772 |
| 4935 | CUUUUUAC U CAAAGAGU | 1996 | ACUCUUUG CUGAUGAGGCCGUUAGGCCGAA IUAAAAAG | 5773 |
| 4937 | UUUUACUC A AAGAGUAU | 1997 | AUACUCUU CUGAUGAGGCCGUUAGGCCGAA IAGUAAAA | 5774 |
| 4952 | AUAUGUUC C CUCCAGGU | 1998 | ACCUGGAG CUGAUGAGGCCGUUAGGCCGAA IAACAUAU | 5775 |
| 4953 | UAUGUUCC C UCCAGGUC | 1999 | GACCUGGA CUGAUGAGGCCGUUAGGCCGAA IGAACAUA | 5776 |
| 4954 | AUGUUCCC U CCAGGUCA | 2000 | UGACCUGG CUGAUGAGGCCGUUAGGCCGAA IGGAACAU | 5777 |
| 4956 | GUUCCCUC C AGGUCAGC | 2001 | GCUGACCU CUGAUGAGGCCGUUAGGCCGAA IAGGGAAC | 5778 |
| 4957 | UUCCCUCC A GGUCAGCU | 2002 | AGCUGACC CUGAUGAGGCCGUUAGGCCGAA IGAGGGAA | 5779 |
| 4962 | UCCAGGUC A GCUGCCCC | 2003 | GGGGCAGC CUGAUGAGGCCGUUAGGCCGAA IACCUGGA | 5780 |
| 4965 | AGGUCAGC U GCCCCCAA | 2004 | UUGGGGGC CUGAUGAGGCCGUUAGGCCGAA ICUGACCU | 5781 |
| 4968 | UCAGCUGC C CCCAAACC | 2005 | GGUUUGGG CUGAUGAGGCCGUUAGGCCGAA ICAGCUGA | 5782 |
| 4969 | CAGCUGCC C CCAAACCC | 2006 | GGGUUUGG CUGAUGAGGCCGUUAGGCCGAA IGCAGCUG | 5783 |
| 4970 | AGCUGCCC C CAAACCCC | 2007 | GGGGUUUG CUGAUGAGGCCGUUAGGCCGAA IGGCAGCU | 5784 |
| 4971 | GCUGCCCC C AAACCCCC | 2008 | GGGGGUUU CUGAUGAGGCCGUUAGGCCGAA IGGGCAGC | 5785 |
| 4972 | CUGCCCCC A AACCCCCU | 2009 | AGGGGGUU CUGAUGAGGCCGUUAGGCCGAA IGGGGCAG | 5786 |
| 4976 | CCCCAAAC C CCCUCCUU | 2010 | AAGGAGGG CUGAUGAGGCCGUUAGGCCGAA IUUUGGGG | 5787 |
| 4977 | CCCAAACC C CCUCCUUA | 2011 | UAAGGAGG CUGAUGAGGCCGUUAGGCCGAA IGUUUGGG | 5788 |
| 4978 | CCAAACCC C CUCCUUAC | 2012 | GUAAGGAG CUGAUGAGGCCGUUAGGCCGAA IGGUUUGG | 5789 |
| 4979 | CAAACCCC C UCCUUACG | 2013 | CGUAAGGA CUGAUGAGGCCGUUAGGCCGAA IGGGUUUG | 5790 |
| 4980 | AAACCCCC U CCUUACGC | 2014 | GCGUAAGG CUGAUGAGGCCGUUAGGCCGAA IGGGGUUU | 5791 |
| 4982 | ACCCCCUC C UUACGCUU | 2015 | AAGCGUAA CUGAUGAGGCCGUUAGGCCGAA IAGGGGGU | 5792 |
| 4983 | CCCCCUCC U UACGCUUU | 2016 | AAAGCGUA CUGAUGAGGCCGUUAGGCCGAA IGAGGGGG | 5793 |
| 4989 | CCUUACGC U UUGUCACA | 2017 | UGUGACAA CUGAUGAGGCCGUUAGGCCGAA ICGUAAGG | 5794 |
| 4995 | GCUUUGUC A CACAAAAA | 2018 | UUUUUGUG CUGAUGAGGCCGUUAGGCCGAA IACAAAGC | 5795 |
| 4997 | UUUGUCAC A CAAAAAGU | 2019 | ACUUUUUG CUGAUGAGGCCGUUAGGCCGAA IUGACAAA | 5796 |
| 4999 | UGUCACAC A AAAAGUGU | 2020 | ACACUUUU CUGAUGAGGCCGUUAGGCCGAA IUGUGACA | 5797 |
| 5009 | AAAGUGUC U CUGCCUUG | 2021 | CAAGGCAG CUGAUGAGGCCGUUAGGCCGAA IACACUUU | 5798 |
| 5011 | AGUGUCUC U GCCUUGAG | 2022 | CUCAAGGC CUGAUGAGGCCGUUAGGCCGAA IAGACACU | 5799 |
| 5014 | GUCUCUGC C UUGAGUCA | 2023 | UGACUCAA CUGAUGAGGCCGUUAGGCCGAA ICAGAGAC | 5800 |
| 5015 | UCUCUGCC U UGAGUCAU | 2024 | AUGACUCA CUGAUGAGGCCGUUAGGCCGAA IGCAGAGA | 5801 |
| 5022 | CUUGAGUC A UCUAUUCA | 2025 | UGAAUAGA CUGAUGAGGCCGUUAGGCCGAA IACUCAAG | 5802 |
| 5025 | GAGUCAUC U AUUCAAGC | 2026 | GCUUGAAU CUGAUGAGGCCGUUAGGCCGAA IAUGACUC | 5803 |
| 5030 | AUCUAUUC A AGCACUUA | 2027 | UAAGUGCU CUGAUGAGGCCGUUAGGCCGAA IAAUAGAU | 5804 |
| 5034 | AUUCAAGC A CUUACAGC | 2028 | GCUGUAAG CUGAUGAGGCCGUUAGGCCGAA ICUUGAAU | 5805 |
| 5036 | UCAAGCAC U UACAGCUC | 2029 | GAGCUGUA CUGAUGAGGCCGUUAGGCCGAA IUGCUUGA | 5806 |
| 5040 | GCACUUAC A GCUCUGGC | 2030 | GCCAGAGC CUGAUGAGGCCGUUAGGCCGAA IUAAGUGC | 5807 |
| 5043 | CUUACAGC U CUGGCCAC | 2031 | GUGGCCAG CUGAUGAGGCCGUUAGGCCGAA ICUGUAAG | 5808 |
| 5045 | UACAGCUC U GGCCACAA | 2032 | UUGUGGCC CUGAUGAGGCCGUUAGGCCGAA IAGCUGUA | 5809 |
| 5049 | GCUCUGGC C ACAACAGG | 2033 | CCUGUUGU CUGAUGAGGCCGUUAGGCCGAA ICCAGAGC | 5810 |
| 5050 | CUCUGGCC A CAACAGGG | 2034 | CCCUGUUG CUGAUGAGGCCGUUAGGCCGAA IGCCAGAG | 5811 |
| 5052 | CUGGCCAC A ACAGGGCA | 2035 | UGCCCUGU CUGAUGAGGCCGUUAGGCCGAA IUGGCCAG | 5812 |
| 5055 | GCCACAAC A GGGCAUUU | 2036 | AAAUGCCC CUGAUGAGGCCGUUAGGCCGAA IUUGUGGC | 5813 |
| 5060 | AACAGGGC A UUUUACAG | 2037 | CUGUAAAA CUGAUGAGGCCGUUAGGCCGAA ICCCUGUU | 5814 |
| 5067 | CAUUUUAC A GGUGCGAA | 2038 | UUCGCACC CUGAUGAGGCCGUUAGGCCGAA IUAAAAUG | 5815 |
| 5080 | CGAAUGAC A GUAGCAUU | 2039 | AAUGCUAC CUGAUGAGGCCGUUAGGCCGAA IUCAUUCG | 5816 |
| 5086 | ACAGUAGC A UUAUGAGU | 2040 | ACUCAUAA CUGAUGAGGCCGUUAGGCCGAA ICUACUGU | 5817 |
| 5106 | GUGAAUUC A GGUAGUAA | 2041 | UUACUACC CUGAUGAGGCCGUUAGGCCGAA IAAUUCAC | 5818 |
| 5124 | UAUGAAAC U AGGGUUUG | 2042 | CAAACCCU CUGAUGAGGCCGUUAGGCCGAA IUUUCAUA | 5819 |
| 5146 | GAUAAUGC U UUCACAAC | 2043 | GUUGUGAA CUGAUGAGGCCGUUAGGCCGAA ICAUUAUC | 5820 |
| 5150 | AUGCUUUC A CAACAUUU | 2044 | AAAUGUUG CUGAUGAGGCCGUUAGGCCGAA IAAAGCAU | 5821 |
| 5152 | GCUUUCAC A ACAUUUGC | 2045 | GCAAAUGU CUGAUGAGGCCGUUAGGCCGAA IUGAAAGC | 5822 |
| 5155 | UUCACAAC A UUUGCAGA | 2046 | UCUGCAAA CUGAUGAGGCCGUUAGGCCGAA IUUGUGAA | 5823 |
| 5161 | ACAUUUGC A GAUGUUUU | 2047 | AAAACAUC CUGAUGAGGCCGUUAGGCCGAA ICAAAUGU | 5824 |
| 5186 | AAAAGUUC C UUCCUAAA | 2048 | UUUAGGAA CUGAUGAGGCCGUUAGGCCGAA IAACUUUU | 5825 |
| 5187 | AAAGUUCC U UCCUAAAA | 2049 | UUUUAGGA CUGAUGAGGCCGUUAGGCCGAA IGAACUUU | 5826 |
| 5190 | GUUCCUUC C UAAAAUAA | 2050 | UUAUUUUA CUGAUGAGGCCGUUAGGCCGAA IAAGGAAC | 5827 |
| 5191 | UUCCUUCC U AAAAUAAU | 2051 | AUUAUUUU CUGAUGAGGCCGUUAGGCCGAA IGAAGGAA | 5828 |
| 5203 | AUAAUUUC U CUACAAUU | 2052 | AAUUGUAG CUGAUGAGGCCGUUAGGCCGAA IAAAUUAU | 5829 |
| 5205 | AAUUUCUC U ACAAUUGG | 2053 | CCAAUUGU CUGAUGAGGCCGUUAGGCCGAA IAGAAAUU | 5830 |
| 5208 | UUCUCUAC A AUUGGAAG | 2054 | CUUCCAAU CUGAUGAGGCCGUUAGGCCGAA IUAGAGAA | 5831 |
| 5229 | GAAGAUUC A GCUAGUUA | 2055 | UAACUAGC CUGAUGAGGCCGUUAGGCCGAA IAAUCUUC | 5832 |
| 5232 | GAUUCAGC U AGUUAGGA | 2056 | UCCUAACU CUGAUGAGGCCGUUAGGCCGAA ICUGAAUC | 5833 |
| 5243 | UUAGGAGC C CAUUUUUU | 2057 | AAAAAAUG CUGAUGAGGCCGUUAGGCCGAA ICUCCUAA | 5834 |
| 5244 | UAGGAGCC C AUUUUUUC | 2058 | GAAAAAAU CUGAUGAGGCCGUUAGGCCGAA IGCUCCUA | 5835 |
| 5245 | AGGAGCCC A UUUUUUCC | 2059 | GGAAAAAA CUGAUGAGGCCGUUAGGCCGAA IGGCUCCU | 5836 |
| 5253 | AUUUUUUC C UAAUCUGU | 2060 | ACAGAUUA CUGAUGAGGCCGUUAGGCCGAA IAAAAAAU | 5837 |
| 5254 | UUUUUUCC U AAUCUGUG | 2061 | CACAGAUU CUGAUGAGGCCGUUAGGCCGAA IGAAAAAA | 5838 |
| 5259 | UCCUAAUC U GUGUGUGC | 2062 | GCACACAC CUGAUGAGGCCGUUAGGCCGAA IAUUAGGA | 5839 |
| 5268 | GUGUGUGC C CUGUAACC | 2063 | GGUUACAG CUGAUGAGGCCGUUAGGCCGAA ICACACAC | 5840 |
| 5269 | UGUGUGCC C UGUAACCU | 2064 | AGGUUACA CUGAUGAGGCCGUUAGGCCGAA IGCACACA | 5841 |
| 5270 | GUGUGCCC U GUAACCUG | 2065 | CAGGUUAC CUGAUGAGGCCGUUAGGCCGAA IGGCACAC | 5842 |
| 5276 | CCUGUAAC C UGACUGGU | 2066 | ACCAGUCA CUGAUGAGGCCGUUAGGCCGAA IUUACAGG | 5843 |
| 5277 | CUGUAACC U GACUGGUU | 2067 | AACCAGUC CUGAUGAGGCCGUUAGGCCGAA IGUUACAG | 5844 |
| 5281 | AACCUGAC U GGUUAACA | 2068 | UGUUAACC CUGAUGAGGCCGUUAGGCCGAA IUCAGGUU | 5845 |
| 5289 | UGGUUAAC A GCAGUCCU | 2069 | AGGACUGC CUGAUGAGGCCGUUAGGCCGAA IUUAACCA | 5846 |
| 5292 | UUAACAGC A GUCCUUUG | 2070 | CAAAGGAC CUGAUGAGGCCGUUAGGCCGAA ICUGUUAA | 5847 |
| 5296 | CAGCAGUC C UUUGUAAA | 2071 | UUUACAAA CUGAUGAGGCCGUUAGGCCGAA IACUGCUG | 5848 |
| 5297 | AGCAGUCC U UUGUAAAC | 2072 | GUUUACAA CUGAUGAGGCCGUUAGGCCGAA IGACUGCU | 5849 |
| 5306 | UUGUAAAC A GUGUUUUA | 2073 | UAAAACAC CUGAUGAGGCCGUUAGGCCGAA IUUUACAA | 5850 |
| 5318 | UUUUAAAC U CUCCUAGU | 2074 | ACUAGGAG CUGAUGAGGCCGUUAGGCCGAA IUUUAAAA | 5851 |
| 5320 | UUAAACUC U CCUAGUCA | 2075 | UGACUAGG CUGAUGAGGCCGUUAGGCCGAA IAGUUUAA | 5852 |
| 5322 | AAACUCUC C UAGUCAAU | 2076 | AUUGACUA CUGAUGAGGCCGUUAGGCCGAA IAGAGUUU | 5853 |
| 5323 | AACUCUCC U AGUCAAUA | 2077 | UAUUGACU CUGAUGAGGCCGUUAGGCCGAA IGAGAGUU | 5854 |
| 5328 | UCCUAGUC A AUAUCCAC | 2078 | GUGGAUAU CUGAUGAGGCCGUUAGGCCGAA IACUAGGA | 5855 |
| 5334 | UCAAUAUC C ACCCCAUC | 2079 | GAUGGGGU CUGAUGAGGCCGUUAGGCCGAA IAUAUUGA | 5856 |
| 5335 | CAAUAUCC A CCCCAUCC | 2080 | GGAUGGGG CUGAUGAGGCCGUUAGGCCGAA IGAUAUUG | 5857 |
| 5337 | AUAUCCAC C CCAUCCAA | 2081 | UUGGAUGG CUGAUGAGGCCGUUAGGCCGAA IUGGAUAU | 5858 |
| 5338 | UAUCCACC C CAUCCAAU | 2082 | AUUGGAUG CUGAUGAGGCCGUUAGGCCGAA IGUGGAUA | 5859 |
| 5339 | AUCCACCC C AUCCAAUU | 2083 | AAUUGGAU CUGAUGAGGCCGUUAGGCCGAA IGGUGGAU | 5860 |
| 5340 | UCCACCCC A UCCAAUUU | 2084 | AAAUUGGA CUGAUGAGGCCGUUAGGCCGAA IGGGUGGA | 5861 |
| 5343 | ACCCCAUC C AAUUUAUC | 2085 | GAUAAAUU CUGAUGAGGCCGUUAGGCCGAA IAUGGGGU | 5862 |
| 5344 | CCCCAUCC A AUUUAUCA | 2086 | UGAUAAAU CUGAUGAGGCCGUUAGGCCGAA IGAUGGGG | 5863 |
| 5352 | AAUUUAUC A AGGAAGAA | 2087 | UUCUUCCU CUGAUGAGGCCGUUAGGCCGAA IAUAAAUU | 5864 |
| 5368 | AAUGGUUC A GAAAAUAU | 2088 | AUAUUUUC CUGAUGAGGCCGUUAGGCCGAA IAACCAUU | 5865 |
| 5381 | AUAUUUUC A GCCUACAG | 2089 | CUGUAGGC CUGAUGAGGCCGUUAGGCCGAA IAAAAUAU | 5866 |
| 5384 | UUUUCAGC C UACAGUUA | 2090 | UAACUGUA CUGAUGAGGCCGUUAGGCCGAA ICUGAAAA | 5867 |
| 5385 | UUUCAGCC U ACAGUUAU | 2091 | AUAACUGU CUGAUGAGGCCGUUAGGCCGAA IGCUGAAA | 5868 |
| 5388 | CAGCCUAC A GUUAUGUU | 2092 | AACAUAAC CUGAUGAGGCCGUUAGGCCGAA IUAGGCUG | 5869 |
| 5398 | UUAUGUUC A GUCACACA | 2093 | UGUGUGAC CUGAUGAGGCCGUUAGGCCGAA IAACAUAA | 5870 |
| 5402 | GUUCAGUC A CACACACA | 2094 | UGUGUGUG CUGAUGAGGCCGUUAGGCCGAA IACUGAAC | 5871 |
| 5404 | UCAGUCAC A CACACAUA | 2095 | UAUGUGUG CUGAUGAGGCCGUUAGGCCGAA IUGACUGA | 5872 |
| 5406 | AGUCACAC A CACAUACA | 2096 | UGUAUGUG CUGAUGAGGCCGUUAGGCCGAA IUGUGACU | 5873 |
| 5408 | UCACACAC A CAUACAAA | 2097 | UUUGUAUG CUGAUGAGGCCGUUAGGCCGAA IUGUGUGA | 5874 |
| 5410 | ACACACAC A UACAAAAU | 2098 | AUUUUGUA CUGAUGAGGCCGUUAGGCCGAA IUGUGUGU | 5875 |
| 5414 | ACACAUAC A AAAUGUUC | 2099 | GAACAUUU CUGAUGAGGCCGUUAGGCCGAA IUAUGUGU | 5876 |
| 5423 | AAAUGUUC C UUUUGCUU | 2100 | AAGCAAAA CUGAUGAGGCCGUUAGGCCGAA IAACAUUU | 5877 |
| 5424 | AAUGUUCC U UUUGCUUU | 2101 | AAAGCAAA CUGAUGAGGCCGUUAGGCCGAA IGAACAUU | 5878 |
| 5430 | CCUUUUGC U UUUAAAGU | 2102 | ACUUUAAA CUGAUGAGGCCGUUAGGCCGAA ICAAAAGG | 5879 |
| 5449 | UUUUUGAC U CCCAGAUC | 2103 | GAUCUGGG CUGAUGAGGCCGUUAGGCCGAA IUCAAAAA | 5880 |
| 5451 | UUUGACUC C CAGAUCAG | 2104 | CUGAUCUG CUGAUGAGGCCGUUAGGCCGAA IAGUCAAA | 5881 |
| 5452 | UUGACUCC C AGAUCAGU | 2105 | ACUGAUCU CUGAUGAGGCCGUUAGGCCGAA IGAGUCAA | 5882 |
| 5453 | UGACUCCC A GAUCAGUC | 2106 | GACUGAUC CUGAUGAGGCCGUUAGGCCGAA IGGAGUCA | 5883 |
| 5458 | CCCAGAUC A GUCAGAGC | 2107 | GCUCUGAC CUGAUGAGGCCGUUAGGCCGAA IAUCUGGG | 5884 |
| 5462 | GAUCAGUC A GAGCCCCU | 2108 | AGGGGCUC CUGAUGAGGCCGUUAGGCCGAA IACUGAUC | 5885 |
| 5467 | GUCAGAGC C CCUACAGC | 2109 | GCUGUAGG CUGAUGAGGCCGUUAGGCCGAA ICUCUGAC | 5886 |
| 5468 | UCAGAGCC C CUACAGCA | 2110 | UGCUGUAG CUGAUGAGGCCGUUAGGCCGAA IGCUCUGA | 5887 |
| 5469 | CAGAGCCC C UACAGCAU | 2111 | AUGCUGUA CUGAUGAGGCCGUUAGGCCGAA IGGCUCUG | 5888 |
| 5470 | AGAGCCCC U ACAGCAUU | 2112 | AAUGCUGU CUGAUGAGGCCGUUAGGCCGAA IGGGCUCU | 5889 |
| 5473 | GCCCCUAC A GCAUUGUU | 2113 | AACAAUGC CUGAUGAGGCCGUUAGGCCGAA IUAGGGGC | 5890 |
| 5476 | CCUACAGC A UUGUUAAG | 2114 | CUUAACAA CUGAUGAGGCCGUUAGGCCGAA ICUGUAGG | 5891 |
| 5504 | UUUUUGUC U CAAUGAAA | 2115 | UUUCAUUG CUGAUGAGGCCGUUAGGCCGAA IACAAAAA | 5892 |
| 5506 | UUUGUCUC A AUGAAAAU | 2116 | AUUUUCAU CUGAUGAGGCCGUUAGGCCGAA IAGACAAA | 5893 |
| 5520 | AAUAAAAC U AUAUUCAU | 2117 | AUGAAUAU CUGAUGAGGCCGUUAGGCCGAA IUUUUAUU | 5894 |

Input Sequence = NM_005228.1.
Cut Site = CH/.
Arm Length = 8.
Core Sequence = CUGAUGAG GCCGUUAGGC CGAA NM_005228.1 (Homo sapiens epidermal growth factor receptor (avian erythroblastic leukemia viral (v-erb-b) oncogene homolog) (EGFR), mRNA)

Underlined region can be any X sequence or linker, as described herein. I=Inosine

TABLE V
Human EGFR Receptor Zinzyme and Substrate Sequence

| | | | | |
|---|---|---|---|---|
| Pos | Substrate | Seq ID | Zinzyme | Seq ID |

| | | | | |
|---|---|---|---|---|
| 9 | GCCGCGCU G CGCCGGAG | 2118 | CUCCGGCG GCCGAAAGGCGAGUGAGGUCU AGCGCGGC | 5895 |
| 11 | CGCGCUGC G CCGGAGUC | 2119 | GACUCCGG GCCGAAAGGCGAGUGAGGUCU GCAGCGCG | 5896 |
| 17 | GCGCCGGA G UCCCGAGC | 2120 | GCUCGGGA GCCGAAAGGCGAGUGAGGUCU UCCGGCGC | 5897 |
| 24 | AGUCCCGA G CUAGCCCC | 2121 | GGGGCUAG GCCGAAAGGCGAGUGAGGUCU UCGGGACU | 5898 |
| 28 | CCGAGCUA G CCCCGGCG | 2122 | CGCCGGGG GCCGAAAGGCGAGUGAGGUCU UAGCUCGG | 5899 |
| 34 | UAGCCCCG G CGCCGCCG | 2123 | CGGCGGCG GCCGAAAGGCGAGUGAGGUCU CGGGGCUA | 5900 |
| 36 | GCCCCGGC G CCGCCGCC | 2124 | GGCGGCGG GCCGAAAGGCGAGUGAGGUCU GCCGGGGC | 5901 |
| 39 | CCGGCGCC G CCGCCGCC | 2125 | GGCGGCGG GCCGAAAGGCGAGUGAGGUCU GGCGCCGG | 5902 |
| 42 | GCGCCGCC G CCGCCCAG | 2126 | CUGGGCGG GCCGAAAGGCGAGUGAGGUCU GGCGGCGC | 5903 |
| 45 | CCGCCGCC G CCCAGACC | 2127 | GGUCUGGG GCCGAAAGGCGAGUGAGGUCU GGCGGCGG | 5904 |
| 63 | GACGACAG G CCACCUCG | 2128 | CGAGGUGG GCCGAAAGGCGAGUGAGGUCU CUGUCGUC | 5905 |
| 71 | GCCACCUC G UCGCCCUC | 2129 | GACCCCGA CCCGAAAGGCGAGUGAGGUCU CAGGUCGC | 5906 |
| 75 | CCUCGUCG G CGUCCGCC | 2130 | GGCGGACG GCCGAAAGGCGAGUGAGGUCU CGACGAGG | 5907 |
| 77 | UCGUCGGC G UCCGCCCG | 2131 | CGGGCGGA GCCGAAAGGCGAGUGAGGUCU GCCGACGA | 5908 |
| 81 | CGGCGUCC G CCCGAGUC | 2132 | GACUCGGG GCCGAAAGGCCAGUGAGGUCU GGACGCCG | 5909 |
| 87 | CCGCCCCA G UCCCCCCC | 2133 | GGCGCGGA GCCGAAAGGCGAGUGAGGUCU UCGGGCGG | 5910 |
| 93 | GAGUCCCC G CCUCGCCG | 2134 | CGGCGAGG GCCCAAAGGCGAGUGAGGUCU GCGGACUC | 5911 |
| 98 | CCCGCCUC G CCGCCAAC | 2135 | CUUGGCGC GCCGAAAGGCGAGUGAGGUCU GAGGCGGG | 5912 |
| 101 | GCCUCGCC G CCAACGCC | 2136 | CGCGUUGG CCCGAAAGGCGAGUGAGGUCU GGCGAGGC | 5913 |
| 107 | CCGCCAAC G CCACAACC | 2137 | GGUUGUGG GCCGAAAGGCGAGUGAGGUCU GUUGGCGG | 5914 |
| 119 | CAACCACC G CGCACGGC | 2138 | GCCGUGCG CCCGAAACGCCACUGACGUCU GGUGGUUG | 5915 |
| 121 | ACCACCGC G CACGGCCC | 2139 | GCGCCGUC GCCGAAAGGCCAGUCAGGUCU GCGCUCCU | 5916 |
| 126 | CCCCCACG G CCCCCUGA | 2140 | UCAGGCGC CCCGAAAGGCGAGUGAGGUCU CGUGCCCC | 5917 |
| 139 | CUGACUCC G UCCAGUAU | 2141 | AUACUGGA GCCGAAAGGCGAGUGAGGUCU GGAGUCAG | 5918 |
| 144 | UCCGUCCA G UAUUGAUC | 2142 | GAUCAAUA GCCGAAAGGCGACUCAGGUCU UCGACGGA | 5919 |
| 159 | UCGGGAGA G CCGGAGCG | 2143 | CGCUCCGG GCCGAAAGGCGAGUGAGGUCU UCUCCCGA | 5920 |
| 165 | GAGCCCCA G CGAGCUCU | 2144 | AGAGCUCG GCCGAAAGGCGAGUGAGGUCU UCCGGCUC | 5921 |
| 169 | CCGACCGA G CUCUUCGG | 2145 | CCGAACAG GCCGAAAGGCCACUCACCUCU UCCCUCCG | 5922 |
| 181 | UUCGGGGA G CAGCCAUG | 2146 | CAUCGCUG CCCGAAACGCGACUGAGCUCU UCCCCGAA | 5923 |
| 184 | CGCCAGCA G CGAUCCCA | 2147 | UCCCAUCG GCCGAAACGCGAGUGAGCUCU UCCUCCCC | 5924 |
| 189 | GCACCCAU G CGACCCUC | 2148 | CACCGUCC CCCCAAAGGCGAGUCACCUCU AUCGCUGC | 5925 |
| 205 | CCCCCACC G CCGGGCCA | 2149 | UGCCCCGG GCCCAAACGCGAGUGACCUCU CGUCCCCG | 5926 |
| 211 | CCGCCCGC G CAGCCCUC | 2150 | GAGCGCUG GCCCAAAGGCCAGUGAGGUCU CCCGGCCG | 5927 |
| 214 | CCCGGGCA G CGCUCCUG | 2151 | CAGGAGCG GCCCAAACGCCAGUGAGGUCU UCCCCCGG | 5928 |
| 216 | GGGCCAGC G CUCCUCGC | 2152 | GCCAGGAC GCCGAAACCCGAGUGAGCUCU GCUGCCCC | 5929 |
| 223 | CGCUCCUG G CGCUGCUC | 2153 | CACCACCG GCCCAAACGCCAGUCACCUCU CACCAGCC | 5930 |
| 225 | CUCCUCCC G CUCCUCCC | 2154 | CCCACCAC CCCCAAACCCCACUCACCUCU CCCACCAC | 5931 |
| 228 | CUGGCCCU G CUGGCUCC | 2155 | CCAGCCAC CCCGAAACGCGAGUCAGGUCU AGCGCCAC | 5932 |
| 232 | CCCUCCUC G CUCCCCUC | 2156 | CACCGCAC CCCCAAACCCCACUCACCUCU CACCACCC | 5933 |
| 235 | UCCUCCCU G CGCUCUCC | 2157 | CCACACCC CCCCAAACGCGAGUGACCUCU ACCCACCA | 5934 |
| 237 | CUCCCUCC G CUCUCCCC | 2158 | CCCCACAC CCCCAAACCCCACUCAGCUCU GCACCCAC | 5935 |
| 242 | UGCGCUCU G CCCCCCCA | 2159 | UCCCCCCC CCCCAAACCCCACUCACGUCU ACACCCCA | 5936 |
| 247 | UCUCCCCC G CCACUCCC | 2160 | CCCACUCC CCCCAAACCCCACUCACCUCU CCCCCACA | 5937 |
| 251 | CCCGGCCA G UCCCCCUC | 2161 | CAGCCCCA CCCCAAACCCCAGUCAGCUCU UCGCCCGC | 5938 |
| 256 | CGAGUCGC G CUCUCCAC | 2162 | CUCCACAC CCCCAAACCCCACUCACCUCU CCCACUCC | 5939 |
| 274 | AAAAGAAA G UUUCCCAA | 2163 | UUGGCAAA CCCCAAACCCCACUCACCUCU UUUCUUUU | 5940 |
| 278 | CAAACUUU G CCAACCCA | 2164 | UGCCUUGG CCCCAAACCCCACUCACCUCU AAACUUUC | 5941 |
| 284 | UUCCCAAC G CACCACUA | 2165 | UACUCCUC CCCCAAAGCCCACUCACCUCU CUUGGCAA | 5942 |
| 290 | AGGCACGA G UAACAAGC | 2166 | GCUUGUUA GCCGAAAGGCGAGUGAGGUCU UCGUGCCU | 5943 |
| 297 | AGUAACAA G CUCACGCA | 2167 | UGCGUGAG GCCGAAAGGCGAGUGAGGUCU UUGUUACU | 5944 |
| 303 | AAGCUCAC G CAGUUGGG | 2168 | CCCAACUG GCCGAAAGGCGAGUGAGGUCU GUGAGCUU | 5945 |
| 306 | CUCACGCA G UUGGGCAC | 2169 | GUGCCCAA GCCGAAAGGCGAGUGAGGUCU UGCGUGAG | 5946 |
| 311 | GCAGUUGG G CACUUUUG | 2170 | CAAAAGUG GCCGAAAGGCGAGUGAGGUCU CCAACUGC | 5947 |
| 335 | UUUUCUCA G CCUCCAGA | 2171 | UCUGGAGG GCCGAAAGGCGAGUGAGGUCU UGAGAAAA | 5948 |
| 348 | CAGAGGAU G UUCAAUAA | 2172 | UUAUUGAA GCCGAAAGGCGAGUGAGGUCU AUCCUCUG | 5949 |
| 359 | CAAUAACU G UGAGGUGG | 2173 | CCACCUCA GCCGAAAGGCGAGUGAGGUCU AGUUAUUG | 5950 |
| 364 | ACUGUGAG G UGGUCCUU | 2174 | AAGGACCA GCCGAAAGGCGAGUGAGGUCU CUCACAGU | 5951 |
| 367 | GUGAGGUG G UCCUUGGG | 2175 | CCCAAGGA GCCGAAAGGCGAGUGAGGUCU CACCUCAC | 5952 |
| 394 | UUACCUAU G UGCAGAGG | 2176 | CCUCUGCA GCCGAAAGGCGAGUGAGGUCU AUAGGUAA | 5953 |
| 396 | ACCUAUGU G CAGAGGAA | 2177 | UUCCUCUG GCCGAAAGGCGAGUGAGGUCU ACAUAGGU | 5954 |
| 439 | UCCAGGAG G UGGCUGGU | 2178 | ACCAGCCA GCCGAAAGGCGAGUGAGGUCU CUCCUGGA | 5955 |
| 442 | AGGAGGUG G CUGGUUAU | 2179 | AUAACCAG GCCGAAAGGCGAGUGAGGUCU CACCUCCU | 5956 |
| 446 | GGUGGCUG G UUAUGUCC | 2180 | GGACAUAA GCCGAAAGGCGAGUGAGGUCU CAGCCACC | 5957 |
| 451 | CUGGUUAU G UCCUCAUU | 2181 | AAUGAGGA GCCGAAAGGCGAGUGAGGUCU AUAACCAG | 5958 |
| 460 | UCCUCAUU G CCCUCAAC | 2182 | GUUGAGGG GCCGAAAGGCGAGUGAGGUCU AAUGAGGA | 5959 |
| 472 | UCAACACA G UGGAGCGA | 2183 | UCGCUCCA GCCGAAAGGCGAGUGAGGUCU UGUGUUGA | 5960 |
| 477 | ACAGUGGA G CGAAUUCC | 2184 | GGAAUUCG GCCGAAAGGCGAGUGAGGUCU UCCACUGU | 5961 |
| 498 | GAAAACCU G CAGAUCAU | 2185 | AUGAUCUG GCCGAAAGGCGAGUGAGGUCU AGGUUUUC | 5962 |
| 519 | GGAAAUAU G UACUACGA | 2186 | UCGUAGUA GCCGAAAGGCGAGUGAGGUCU AUAUUUCC | 5963 |
| 538 | AUUCCUAU G CCUUAGCA | 2187 | UGCUAAGG GCCGAAAGGCGAGUGAGGUCU AUAGGAAU | 5964 |
| 544 | AUGCCUUA G CAGUCUUA | 2188 | UAAGACUG GCCGAAAGGCGAGUGAGGUCU UAAGGCAU | 5965 |
| 547 | CCUUAGCA G UCUUAUCU | 2189 | AGAUAAGA GCCGAAAGGCGAGUGAGGUCU UGCUAAGG | 5966 |
| 565 | ACUAUGAU G CAAAUAAA | 2190 | UUUAUUUG GCCGAAAGGCGAGUGAGGUCU AUCAUAGU | 5967 |
| 588 | CUGAAGGA G CUGCCCAU | 2191 | AUGGGCAG GCCGAAAGGCGAGUGAGGUCU UCCUUCAG | 5968 |
| 591 | AAGGAGCU G CCCAUGAG | 2192 | CUCAUGGG GCCGAAAGGCGAGUGAGGUCU AGCUCCUU | 5969 |
| 618 | GAAAUCCU G CAUGGCGC | 2193 | GCGCCAUG GCCGAAAGGCGAGUGAGGUCU AGGAUUUC | 5970 |
| 623 | CCUGCAUG G CGCCGUGC | 2194 | GCACGGCG GCCGAAAGGCGAGUGAGGUCU CAUGCAGG | 5971 |
| 625 | UGCAUGGC G CCGUGCGG | 2195 | CCGCACGG GCCGAAAGGCGAGUGAGGUCU GCCAUGCA | 5972 |
| 628 | AUGGCGCC G UGCGGUUC | 2196 | GAACCGCA GCCGAAAGGCGAGUGAGGUCU GGCGCCAU | 5973 |
| 630 | GGCGCCGU G CGGUUCAG | 2197 | CUGAACCG GCCGAAAGGCGAGUGAGGUCU ACGGCGCC | 5974 |
| 633 | GCCGUGCG G UUCAGCAA | 2198 | UUGCUGAA GCCGAAAGGCGAGUGAGGUCU CGCACGGC | 5975 |
| 638 | GCGGUUCA G CAACAACC | 2199 | GGUUGUUG GCCGAAAGGCGAGUGAGGUCU UGAACCGC | 5976 |
| 649 | ACAACCCU G CCCUGUGC | 2200 | GCACAGGG GCCGAAAGGCGAGUGAGGUCU AGGGUUGU | 5977 |
| 654 | CCUGCCCU G UGCAACGU | 2201 | ACGUUGCA GCCGAAAGGCGAGUGAGGUCU AGGGCAGG | 5978 |
| 656 | UGCCCUGU G CAACGUGG | 2202 | CCACGUUG GCCGAAAGGCGAGUGAGGUCU ACAGGGCA | 5979 |
| 661 | UGUGCAAC G UGGAGAGC | 2203 | GCUCUCCA GCCGAAAGGCGAGUGAGGUCU GUUGCACA | 5980 |
| 668 | CGUGGAGA G CAUCCAGU | 2204 | ACUGGAUG GCCGAAAGGCGAGUGAGGUCU UCUCCACG | 5981 |
| 675 | AGCAUCCA G UGGCGGGA | 2205 | UCCCGCCA GCCGAAAGGCGAGUGAGGUCU UGGAUGCU | 5982 |
| 678 | AUCCAGUG G CGGGACAU | 2206 | AUGUCCCG GCCGAAAGGCGAGUGAGGUCU CACUGGAU | 5983 |
| 688 | GGGACAUA G UCAGCAGU | 2207 | ACUGCUGA GCCGAAAGGCGAGUGAGGUCU UAUGUCCC | 5984 |
| 692 | CAUAGUCA G CAGUGACU | 2208 | AGUCACUG GCCGAAAGGCGAGUGAGGUCU UGACUAUG | 5985 |
| 695 | AGUCAGCA G UGACUUUC | 2209 | GAAAGUCA GCCGAAAGGCGAGUGAGGUCU UGCUGACU | 5986 |
| 707 | CUUUCUCA G CAACAUGU | 2210 | ACAUGUUG GCCGAAAGGCGAGUGAGGUCU UGAGAAAG | 5987 |
| 714 | AGCAACAU G UCGAUGGA | 2211 | UCCAUCGA GCCGAAAGGCGAGUGAGGUCU AUGUUGCU | 5988 |
| 740 | CCACCUGG G CAGCUGCC | 2212 | GGCAGCUG GCCGAAAGGCGAGUGAGGUCU CCAGGUGG | 5989 |
| 743 | CCUGGGCA G CUGCCAAA | 2213 | UUUGGCAG GCCGAAAGGCGAGUGAGGUCU UGCCCAGG | 5990 |
| 746 | GGGCAGCU G CCAAAAGU | 2214 | ACUUUUGG GCCGAAAGGCGAGUGAGGUCU AGCUGCCC | 5991 |
| 753 | UGCCAAAA G UGUGAUCC | 2215 | GGAUCACA GCCGAAAGGCGAGUGAGGUCU UUUUGGCA | 5992 |
| 755 | CCAAAAGU G UGAUCCAA | 2216 | UUGGAUCA GCCGAAAGGCGAGUGAGGUCU ACUUUUGG | 5993 |
| 764 | UGAUCCAA G CUGUCCCA | 2217 | UGGGACAG GCCGAAAGGCGAGUGAGGUCU UUGGAUCA | 5994 |
| 767 | UCCAAGCU G UCCCAAUG | 2218 | CAUUGGGA GCCGAAAGGCGAGUGAGGUCU AGCUUGGA | 5995 |
| 779 | CAAUGGGA G CUGCUGGG | 2219 | CCCAGCAG GCCGAAAGGCGAGUGAGGUCU UCCCAUUG | 5996 |
| 782 | UGGGAGCU G CUGGGGUG | 2220 | CACCCCAG GCCGAAAGGCGAGUGAGGUCU AGCUCCCA | 5997 |
| 788 | CUGCUGGG G UGCAGGAG | 2221 | CUCCUGCA GCCGAAAGGCGAGUGAGGUCU CCCAGCAG | 5998 |
| 790 | GCUGGGGU G CAGGAGAG | 2222 | CUCUCCUG GCCGAAAGGCGAGUGAGGUCU ACCCCAGC | 5999 |
| 806 | GGAGAACU G CCAGAAAC | 2223 | GUUUCUGG GCCGAAAGGCGAGUGAGGUCU AGUUCUCC | 6000 |
| 830 | AAUCAUCU G UGCCCAGC | 2224 | GCUGGGCA GCCGAAAGGCGAGUGAGGUCU AGAUGAUU | 6001 |
| 832 | UCAUCUGU G CCCAGCAG | 2225 | CUGCUGGG GCCGAAAGGCGAGUGAGGUCU ACAGAUGA | 6002 |
| 837 | UGUGCCCA G CAGUGCUC | 2226 | GAGCACUG GCCGAAAGGCGAGUGAGGUCU UGGGCACA | 6003 |
| 840 | GCCCAGCA G UGCUCCGG | 2227 | CCGGAGCA GCCGAAAGGCGAGUGAGGUCU UGCUGGGC | 6004 |
| 842 | CCAGCAGU G CUCCGGGC | 2228 | GCCCGGAG GCCGAAAGGCGAGUGAGGUCU ACUGCUGG | 6005 |
| 849 | UGCUCCGG G CGCUGCCG | 2229 | CGGCAGCG GCCGAAAGGCGAGUGAGGUCU CCGGAGCA | 6006 |
| 851 | CUCCGGGC G CUGCCGUG | 2230 | CACGGCAG GCCGAAAGGCGAGUGAGGUCU GCCCGGAG | 6007 |
| 854 | CGGGCGCU G CCGUGGCA | 2231 | UGCCACGG GCCGAAAGGCGAGUGAGGUCU AGCGCCCG | 6008 |
| 857 | GCGCUGCC G UGGCAAGU | 2232 | ACUUGCCA GCCGAAAGGCGAGUGAGGUCU GGCAGCGC | 6009 |
| 860 | CUGCCGUG G CAAGUCCC | 2233 | GGGACUUG GCCGAAAGGCGAGUGAGGUCU CACGGCAG | 6010 |
| 864 | CGUGGCAA G UCCCCCAG | 2234 | CUGGGGGA GCCGAAAGGCGAGUGAGGUCU UUGCCACG | 6011 |
| 872 | GUCCCCCA G UGACUGCU | 2235 | AGCAGUCA GCCGAAAGGCGAGUGAGGUCU UGGGGGAC | 6012 |
| 878 | CAGUGACU G CUGCCACA | 2236 | UGUGGCAG GCCGAAAGGCGAGUGAGGUCU AGUCACUG | 6013 |
| 881 | UGACUGCU G CCACAACC | 2237 | GGUUGUGG GCCGAAAGGCGAGUGAGGUCU AGCAGUCA | 6014 |
| 891 | CACAACCA G UGUGCUGC | 2238 | GCAGCACA GCCGAAAGGCGAGUGAGGUCU UGGUUGUG | 6015 |
| 893 | CAACCAGU G UGCUGCAG | 2239 | CUGCAGCA GCCGAAAGGCGAGUGAGGUCU ACUGGUUG | 6016 |
| 895 | ACCAGUGU G CUGCAGGC | 2240 | GCCUGCAG GCCGAAAGGCGAGUGAGGUCU ACACUGGU | 6017 |
| 898 | AGUGUGCU G CAGGCUGC | 2241 | GCAGCCUG GCCGAAAGGCGAGUGAGGUCU AGCACACU | 6018 |
| 902 | UGCUGCAG G CUCCACAG | 2242 | CUGUGCAG GCCGAAAGGCGAGUGAGGUCU CUGCAGCA | 6019 |
| 905 | UGCAGGCU G CACAGGCC | 2243 | GGCCUGUG GCCGAAAGGCGAGUGAGGUCU AGCCUGCA | 6020 |
| 911 | CUGCACAG G CCCCCGGG | 2244 | CCCGGGGG GCCGAAAGGCGAGUGAGGUCU CUGUGCAG | 6021 |
| 923 | CCGGGAGA G CGACUGCC | 2245 | GGCAGUCG GCCGAAAGGCGAGUGAGGUCU UCUCCCGG | 6022 |
| 929 | GAGCGACU G CCUGGUCU | 2246 | AGACCAGG GCCGAAAGGCGAGUGAGGUCU AGUCGCUC | 6023 |
| 934 | ACUGCCUG G UCUGCCGC | 2247 | GCGGCAGA GCCGAAAGGCGAGUGAGGUCU CAGGCAGU | 6024 |
| 938 | CCUGGUCU G CCGCAAAU | 2248 | AUUUGCGG GCCGAAAGGCGAGUGAGGUCU AGACCAGG | 6025 |
| 941 | GGUCUGCC G CAAAUUCC | 2249 | GGAAUUUG GCCGAAAGGCGAGUGAGGUCU GGCAGACC | 6026 |
| 958 | GAGACGAA G CCACGUGC | 2250 | GCACGUGG GCCGAAAGGCGAGUGAGGUCU UUCGUCUC | 6027 |
| 963 | GAAGCCAC G UGCAAGGA | 2251 | UCCUUGCA GCCGAAAGGCGAGUGAGGUCU GUGGCUUC | 6028 |
| 965 | AGCCACGU G CAAGGACA | 2252 | UGUCCUUG GCCGAAAGGCGAGUGAGGUCU ACGUGGCU | 6029 |
| 977 | GGACACCU G CCCCCCAC | 2253 | GUGGGGGG GCCGAAAGGCGAGUGAGGUCU AGGUGUCC | 6030 |
| 990 | CCACUCAU G CUCUACAA | 2254 | UUGUAGAG GCCGAAAGGCGAGUGAGGUCU AUGAGUGG | 6031 |
| 1008 | CCCACCAC G UACCAGAU | 2255 | AUCUGGUA GCCGAAAGGCGAGUGAGGUCU GUGGUGGG | 6032 |
| 1021 | AGAUGGAU G UGAACCCC | 2256 | GGGGUUCA GCCGAAAGGCGAGUGAGGUCU AUCCAUCU | 6033 |
| 1034 | CCCCGAGG G CAAAUACA | 2257 | UGUAUUUG GCCGAAAGGCGAGUGAGGUCU CCUCGGGG | 6034 |
| 1043 | CAAAUACA G CUUUGGUG | 2258 | CACCAAAG GCCGAAAGGCGAGUGAGGUCU UGUAUUUG | 6035 |
| 1049 | CAGCUUUG G UGCCACCU | 2259 | AGGUGGCA GCCGAAAGGCGAGUGAGGUCU CAAAGCUG | 6036 |
| 1051 | GCUUUGGU G CCACCUGC | 2260 | GCAGGUGG GCCGAAAGGCGAGUGAGGUCU ACCAAAGC | 6037 |
| 1058 | UGCCACCU G CGUGAAGA | 2261 | UCUUCACG GCCGAAAGGCGAGUGAGGUCU AGGUGGCA | 6038 |
| 1060 | CCACCUGC G UGAAGAAG | 2262 | CUUCUUCA GCCGAAAGGCGAGUGAGGUCU GCAGGUGG | 6039 |
| 1068 | GUGAAGAA G UGUCCCCG | 2263 | CGGGGACA GCCGAAAGGCGAGUGAGGUCU UUCUUCAC | 6040 |
| 1070 | GAAGAAGU G UCCCCGUA | 2264 | UACGGGGA GCCGAAAGGCGAGUGAGGUCU ACUUCUUC | 6041 |
| 1076 | GUGUCCCC G UAAUUAUG | 2265 | CAUAAUUA GCCGAAAGGCGAGUGAGGUCU GGGGACAC | 6042 |
| 1084 | GUAAUUAU G UGGUGACA | 2266 | UGUCACCA GCCGAAAGGCGAGUGAGGUCU AUAAUUAC | 6043 |
| 1087 | AUUAUGUG G UGACAGAU | 2267 | AUCUGUCA GCCGAAAGGCGAGUGAGGUCU CACAUAAU | 6044 |
| 1100 | AGAUCACG G CUCGUGCG | 2268 | CGCACGAG GCCGAAAGGCGAGUGAGGUCU CGUGAUCU | 6045 |
| 1104 | CACGGCUC G UGCGUCCG | 2269 | CGGACGCA GCCGAAAGGCGAGUGAGGUCU GAGCCGUG | 6046 |
| 1106 | CGGCUCGU G CGUCCGAG | 2270 | CUCGGACG GCCGAAAGGCGAGUGAGGUCU ACGAGCCG | 6047 |
| 1108 | GCUCGUGC G UCCGAGCC | 2271 | GGCUCGGA GCCGAAAGGCGAGUGAGGUCU GCACGAGC | 6048 |
| 1114 | GCGUCCGA G CCUGUGGG | 2272 | CCCACAGG GCCGAAAGGCGAGUGAGGUCU UCGGACGC | 6049 |
| 1118 | CCGAGCCU G UGGGGCCG | 2273 | CGGCCCCA GCCGAAAGGCGAGUGAGGUCU AGGCUCGG | 6050 |
| 1123 | CCUGUGGG G CCGACAGC | 2274 | GCUGUCGG GCCGAAAGGCGAGUGAGGUCU CCCACAGG | 6051 |
| 1130 | GCCCGACA G CUAUGAGA | 2275 | UCUCAUAC GCCGAAAGGCGAGUGAGGUCU UGUCCGCC | 6052 |
| 1151 | CGAACACC G CGUCCGCA | 2276 | UGCGGACG GCCGAAAGGCGACUCAGGUCU CCUCUUCC | 6053 |
| 1153 | AAGACGGC G UCCGCAAG | 2277 | CUUGCCCA GCCGAAAGGCGAGUGAGGUCU GCCGUCUU | 6054 |
| 1157 | CGGCCUCC G CAAGUGUA | 2278 | UACACUUG GCCGAAAGGCGACUCAGGUCU GCACGCCC | 6055 |
| 1161 | CUCCGCAA G UGUAACAA | 2279 | UUCUUACA GCCCAAAGGCGAGUGAGGUCU UUGCCCAC | 6056 |
| 1163 | CCGCAACU G UAAGAAGU | 2280 | ACUUCUUA GCCCAAAGGCGAGUGAGGUCU ACUUGCGG | 6057 |
| 1170 | UGUAAGAA G UGCGAAGG | 2281 | CCUUCGCA GCCGAAAGGCGAGUGAGGUCU UUCUUACA | 6058 |
| 1172 | UAACAACU G CGAAGCGC | 2282 | GCCCUUCG GCCCAAAGGCGAGUGAGGUCU ACUUCUUA | 6059 |
| 1179 | UGCGAAGG G CCUUGCCG | 2283 | CGCCAACG GCCGAAAGGCGAGUGAGGUCU CCUUCGCA | 6060 |
| 1184 | ACGGCCUU G CCCCAAAG | 2284 | CUUUGCCG GCCGAAAGGCCAGUCAGGUCU AACGCCCU | 6061 |
| 1187 | GCCUUGCC G CAAAGUGU | 2285 | ACACUUUG GCCCAAACGCGAGUCAGGUCU GGCAAGGC | 6062 |
| 1192 | GCCCCAAA G UGUCUAAC | 2286 | GUUACACA GCCGAAAGCCGAGUCAGGUCU UUUGCCCC | 6063 |
| 1194 | CCCAAACU G UGUAACGG | 2287 | CCCUUACA GCCGAAAGGCGAGUGAGGUCU ACUUUCCC | 6064 |
| 1196 | CAAACUGU G UAACGGAA | 2288 | UUCCGUUA GCCCAAAGGCGAGUGAGGUCU ACACUUUG | 6065 |
| 1208 | CGGAAUAG G UAUUGGUG | 2289 | CACCAAUA GCCGAAACGCGACUGAGGUCU CUAUUCCG | 6066 |
| 1214 | ACCUAUUG G UGAAUUUA | 2290 | UAAAUUCA GCCCAAACGCGACUCAGGUCU CAAUACCU | 6067 |
| 1243 | CCAUAAAU G CUACGAAU | 2291 | AUUCGUAG GCCGAAACCCGAGUGAGGUCU AUUUAUCG | 6068 |
| 1271 | CAAAAACU G CACCUCCA | 2292 | UGGAGGUG GCCGAAAGGCGAGUGAGGUCU AGUUUUUG | 6069 |
| 1283 | CUCCAUCA G UGCCCAUC | 2293 | GAUCGCCA GCCCAAAGGCGACUCAGGUCU UGAUCCAG | 6070 |
| 1286 | CAUCACUC G CCAUCUCC | 2294 | CCACAUCG GCCGAAACCCGACUCAGGUCU CACUGAUC | 6071 |
| 1302 | CACAUCCU G CCGCUCGC | 2295 | GCCACCGG GCCGAAAGGCCACUCAGGUCU AGGAUGUC | 6072 |
| 1306 | UCCUGCCC G UCGCAUUU | 2296 | AAAUGCCA GCCGAAAGGCGACUCAGGUCU CCGCACGA | 6073 |
| 1309 | UCCCCCUG G CAUUUAGG | 2297 | CCUAAAUG GCCGAAAGGCGAGUCAGGUCU CACCGGCA | 6074 |
| 1319 | AUUUAGGG G UGACUCCU | 2298 | AGGAGUCA GCCGAAACCCGAGUGAGGUCU CCCUAAAU | 6075 |
| 1378 | UGAAAACC G UAAACGAA | 2299 | UUCCUUUA GCCCAAAGGCGAGUGAGGUCU GGUUUUCA | 6076 |
| 1395 | AUCACACC G UUUUUGCU | 2300 | AGCAAAAA GCCCAAAGGCGAGUGAGGUCU CCUGUGAU | 6077 |
| 1401 | GGGUUUUU G CUCAUUCA | 2301 | UCAAUCAG GCCGAAAGGCGAGUGAGGUCU AAAAACCC | 6078 |
| 1411 | UGAUUCAC G CUUGCCCU | 2302 | ACGCCAAC GCCCAAAGGCGAGUGAGGUCU CUGAAUCA | 6079 |
| 1416 | CAGGCUUG G CCUGAAAA | 2303 | UUUUCAGG GCCGAAACCCGAGUCAGGUCU CAAGCCUG | 6080 |
| 1441 | ACCUCCAU G CCUUUGAG | 2304 | CUCAAAGG GCCGAAAGGCGAGUGAGGUCU AUGGAGGU | 6081 |
| 1466 | AAUCAUAC G CCGCACCA | 2305 | UCCUGCCC GCCGAAAGGCGAGUGAGGUCU GUAUGAUU | 6082 |
| 1469 | CAUACGCG G CAGGACCA | 2306 | UGGUCCUG GCCGAAAGGCGAGUGAGGUCU CGCGUAUG | 6083 |
| 1479 | ACCACCAA G CAACAUGG | 2307 | CCAUGUUG GCCGAAACGCGAGUGAGGUCU UUCGUCCU | 6084 |
| 1487 | CCAACAUC G UCAGUUUU | 2308 | AAAACUGA GCCGAAACGCGAGUGAGGUCU CAUGUUCC | 6085 |
| 1491 | CAUGGUCA G UUUUCUCU | 2309 | ACACAAAA GCCCAAAGGCGAGUGAGGUCU UGACCAUC | 6086 |
| 1501 | UUUCUCUU G CAGUCGUC | 2310 | GACCACUG GCCGAAAGGCGAGUGAGGUCU AACAGAAA | 6087 |
| 1504 | CUCUUCCA G UCGUCAGC | 2311 | CCUGACGA GCCGAAAGGCCACUCAGGUCU UGCAAGAG | 6088 |
| 1507 | UUGCAGUC G UCAGCCUG | 2312 | CAGGCUGA GCCGAAAGGCCAGUGAGGUCU GACUCCAA | 6089 |
| 1511 | AGUCGUCA G CCUCAACA | 2313 | UGUUCACG GCCGAAAGGCGACUGAGGUCU UGACGACU | 6090 |
| 1538 | GCGAUUAC G CUCCCUCA | 2314 | UGAGGGAG GCCCAAAGGCGAGUGAGGUCU GUAAUCCC | 6091 |
| 1556 | GGAGAUAA G UGAUGGAG | 2315 | CUCCAUCA GCCCAAAGGCGAGUGAGGUCU UUAUCUCC | 6092 |
| 1567 | AUGGAGAU G UGAUAAUU | 2316 | AAUUAUCA GCCCAAACGCGAGUGAGGUCU AUCUCCAU | 6093 |
| 1593 | AAAAAUUU G UGCUAUGC | 2317 | GCAUACCA GCCGAAAGCCCACUCAGGUCU AAAUUUUU | 6094 |
| 1595 | AAAUUUGU G CUAUGCAA | 2318 | UUGCAUAC GCCGAAAGGCCAGUGAGGUCU ACAAAUUU | 6095 |
| 1600 | UGUGCUAU G CAAAUACA | 2319 | UGUAUUUG GCCGAAAGGCGAGUGAGGUCU AUAGCACA | 6096 |
| 1626 | AAAAAACU G UUUGGGAC | 2320 | GUCCCAAA GCCGAAAGGCGAGUGAGGUCU AGUUUUUU | 6097 |
| 1640 | GACCUCCG G UCAGAAAA | 2321 | UUUUCUGA GCCGAAAGGCGAGUGAGGUCU CGGAGGUC | 6096 |
| 1661 | AAUUAUAA G CAACAGAG | 2322 | CUCUGUUG GCCGAAAGGCGAGUGAGGUCU UUAUAAUU | 6099 |
| 1670 | CAACAGAG G UGAAAACA | 2323 | UGUUUUCA GCCGAAAGGCGAGUGAGGUCU CUCUGUUG | 6100 |
| 1679 | UGAAAACA G CUGCAAGG | 2324 | CCUUGCAG GCCGAAAGGCGAGUGAGGUCU UGUUUUCA | 6101 |
| 1682 | AAACAGCU G CAAGGCCA | 2325 | UGGCCUUG GCCGAAAGGCGAGUGAGGUCU AGCUGUUU | 6102 |
| 1687 | GCUGCAAG G CCACAGGC | 2326 | GCCUGUGG GCCGAAAGGCGAGUGAGGUCU CUUGCAGC | 6103 |
| 1694 | GGCCACAG G CCAGGUCU | 2327 | AGACCUGG GCCGAAAGGCGAGUGAGGUCU CUGUGGCC | 6104 |
| 1699 | CAGGCCAG G UCUGCCAU | 2328 | AUGGOAGA GCCGAAAGGCGAGUGAGGUCU CUGGCCUG | 6105 |
| 1703 | CCAGGUCU G CCAUGCCU | 2329 | AGGCAUGG GCCGAAAGGCGAGUGAGGUCU AGACCUGG | 6106 |
| 1708 | UCUGCCAU G CCUUGUGC | 2330 | GCACAAGG GCCGAAAGGCGAGUGAGGUCU AUGGCAGA | 6107 |
| 1713 | CAUGCCUU G UGCUCCCC | 2331 | GGGGAGCA GCCGAAAGGCGAGUGAGGUCU AAGGCAUG | 6108 |
| 1715 | UGCCUUGU G CUCCCCCG | 2332 | CGGGGGAG GCCGAAAGGCGAGUGAGGUCU ACAAGGCA | 6109 |
| 1727 | CCCCGAGG G CUGCUGGG | 2333 | CCCAGCAG GCCGAAAGGCGAGUGAGGUCU CCUCGGGG | 6110 |
| 1730 | CGAGGGCU G CUGGGGCC | 2334 | GGCCCCAG GCCGAAAGGCGAGUGAGGUCU AGCCCUCG | 6111 |
| 1736 | CUGCUGGG G CCCGGAGC | 2335 | GCUCCGGG GCCGAAAGGCGAGUGAGGUCU CCCAGCAG | 6112 |
| 1743 | GGCCCGGA G CCCAGGGA | 2336 | UCCCUGGG GCCGAAAGGCGAGUGAGGUCU UCCGGGCC | 6113 |
| 1754 | CAGGGACU G CGUCUCUU | 2337 | AAGAGACG GCCGAAAGGCGAGUGAGGUCU AGUCCCUG | 6114 |
| 1756 | GGGACUGC G UCUCUUGC | 2338 | GCAAGAGA GCCGAAAGGCGAGUGAGGUCU GCAGUCCC | 6115 |
| 1763 | CGUCUCUU G CCGGAAUG | 2339 | CAUUCCGG GCCGAAAGGCGAGUGAGGUCU AAGAGACG | 6116 |
| 1771 | GCCGGAAU G UCAGCCGA | 2340 | UCGGCUGA GCCGAAAGGCGAGUGAGGUCU AUUCCGGC | 6117 |
| 1775 | GAAUGUCA G CCGAGGCA | 2341 | UGCCUCGG GCCGAAAGGCGAGUGAGGUCU UGACAUUC | 6118 |
| 1781 | CAGCCGAG G CAGGGAAU | 2342 | AUUCCCUG GCCGAAAGGCGAGUGAGGUCU CUCGGCUG | 6119 |
| 1790 | CAGGGAAU G CGUGGACA | 2343 | UGUCCACG GCCGAAAGGCGAGUGAGGUCU AUUCCCUG | 6120 |
| 1792 | GGGAAUGC G UGGACAAG | 2344 | CUUGUCCA GCCGAAAGGCGAGUGAGGUCU GCAUUCCC | 6121 |
| 1800 | GUGGACAA G UGCAAGCU | 2345 | AGCUUGCA GCCGAAAGGCGAGUGAGGUCU UUGUCCAC | 6122 |
| 1802 | GGACAAGU G CAAGCUUC | 2346 | GAAGCUUG GCCGAAAGGCGAGUGAGGUCU ACUUGUCC | 6123 |
| 1806 | AAGUGCAA G CUUCUGGA | 2347 | UCCAGAAG GCCGAAAGGCGAGUGAGGUCU UUGCACUU | 6124 |
| 1817 | UCUGGAGG G UGAGCCAA | 2348 | UUGGCUCA GCCGAAAGGCGAGUGAGGUCU CCUCCAGA | 6125 |
| 1821 | GAGGGUGA G CCAAGGGA | 2349 | UCCCUUGG GCCGAAAGGCGAGUGAGGUCU UCACCCUC | 6126 |
| 1830 | CCAAGGGA G UUUGUGGA | 2350 | UCCACAAA GCCGAAAGGCGAGUGAGGUCU UCCCUUGG | 6127 |
| 1834 | GGGAGUUU G UGGAGAAC | 2351 | GUUCUCCA GCCGAAAGGCGAGUGAGGUCU AAACUCCC | 6128 |
| 1848 | AACUCUGA G UGCAUACA | 2352 | UGUAUGCA GCCGAAAGGCGAGUGAGGUCU UCAGAGUU | 6129 |
| 1850 | CUCUGAGU G CAUACAGU | 2353 | ACUGUAUG GCCGAAAGGCGAGUGAGGUCU ACUCAGAG | 6130 |
| 1857 | UGCAUACA G UGCCACCC | 2354 | GGGUGGCA GCCGAAAGGCGAGUGAGGUCU UGUAUGCA | 6131 |
| 1859 | CAUACAGU G CCACCCAG | 2355 | CUGGGUGG GCCGAAAGGCGAGUGAGGUCU ACUGUAUG | 6132 |
| 1869 | CACCCAGA G UGCCUGCC | 2356 | GGCAGGCA GCCGAAAGGCGAGUGAGGUCU UCUGGGUG | 6133 |
| 1871 | CCCAGAGU G CCUGCCUC | 2357 | GAGGCAGG GCCGAAAGGCGAGUGAGGUCU ACUCUGGG | 6134 |
| 1875 | GAGUGCCU G CCUCAGGC | 2358 | GCCUGAGG GCCGAAAGGCGAGUGAGGUCU AGGCACUC | 6135 |
| 1882 | UGCCUCAG G CCAUGAAC | 2359 | GUUCAUGG GCCGAAAGGCGAGUGAGGUCU CUGAGGCA | 6136 |
| 1898 | CAUCACCU G CACAGGAC | 2360 | GUCCUGUG GCCGAAAGGCGAGUGAGGUCU AGGUGAUG | 6137 |
| 1922 | AGACAACU G UAUCCAGU | 2361 | ACUGGAUA GCCGAAAGGCGAGUGAGGUCU AGUUGUCU | 6138 |
| 1929 | UGUAUCCA G UGUGCCCA | 2362 | UGGGCACA GCCGAAAGGCGAGUGAGGUCU UGGAUACA | 6139 |
| 1931 | UAUCCAGU G UGCCCACU | 2363 | AGUGGGCA GCCGAAAGGCGAGUGAGGUCU ACUGGAUA | 6140 |
| 1933 | UCCAGUGU G CCCACUAC | 2364 | GUAGUGGG GCCGAAAGGCGAGUGAGGUCU ACACUGGA | 6141 |
| 1949 | CAUUGACG G CCCCCACU | 2365 | AGUGGGGG GCCGAAAGGCGAGUGAGGUCU CGUCAAUG | 6142 |
| 1958 | CCCCCACU G CGUCAAGA | 2366 | UCUUGACG GCCGAAAGGCGAGUGAGGUCU AGUGGGGG | 6143 |
| 1960 | CCCACUGC G UCAAGACC | 2367 | GGUCUUGA GCCGAAAGGCGAGUGAGGUCU GCAGUGGG | 6144 |
| 1970 | CAAGACCU G CCCGGCAG | 2368 | CUGCCGGG GCCGAAAGGCGAGUGAGGUCU AGGUCUUG | 6145 |
| 1975 | CCUGCCCG G CAGGAGUC | 2369 | GACUCCUG GCCGAAAGGCGAGUGAGGUCU CGGGCAGG | 6146 |
| 1981 | CGGCAGGA G UCAUGGGA | 2370 | UCCCAUGA GCCGAAAGGCGAGUGAGGUCU UCCUGCCG | 6147 |
| 2005 | ACACCCUG G UCUGGAAG | 2371 | CUUCCAGA GCCGAAAGGCGAGUGAGGUCU CAGGGUGU | 6148 |
| 2013 | GUCUGGAA G UACGCAGA | 2372 | UCUGCGUA GCCGAAAGGCGAGUGAGGUCU UUCCAGAC | 6149 |
| 2017 | GGAAGUAC G CAGACGCC | 2373 | GGCGUCUG GCCGAAAGGCGAGUGAGGUCU GUACUUCC | 6150 |
| 2023 | ACGCAGAC G CCGGCCAU | 2374 | AUGGCCGG GCCGAAAGGCGAGUGAGGUCU GUCUGCGU | 6151 |
| 2027 | AGACGCCG G CCAUGUGU | 2375 | ACACAUGG GCCGAAAGGCGAGUGAGGUCU CGGCGUCU | 6152 |
| 2032 | CCGGCCAU G UGUGCCAC | 2376 | GUGGCACA GCCGAAAGGCGAGUGAGGUCU AUGGCCGG | 6153 |
| 2034 | GGCCAUGU G UGCCACCU | 2377 | AGGUGGCA GCCGAAAGGCGAGUGAGGUCU ACAUGGCC | 6154 |
| 2036 | CCAUGUGU G CCACCUGU | 2378 | ACAGGUGG GCCGAAAGGCGAGUGAGGUCU ACACAUGG | 6155 |
| 2043 | UGCCACCU G UGCCAUCC | 2379 | GGAUGGCA GCCGAAAGGCGAGUGAGGUCU AGGUGGCA | 6156 |
| 2045 | CCACCUGU G CCAUCCAA | 2380 | UUGGAUGG GCCGAAAGGCGAGUGAGGUCU ACAGGUGG | 6157 |
| 2057 | UCCAAACU G CACCUACG | 2381 | CGUAGGUG GCCGAAAGGCGAGUGAGGUCU AGUUUGGA | 6158 |
| 2069 | CUACGGAU G CACUGGGC | 2382 | GCCCAGUG GCCGAAAGGCGAGUGAGGUCU AUCCGUAG | 6159 |
| 2076 | UGCACUGG G CCAGGUCU | 2383 | AGACCUGG GCCGAAAGGCGAGUGAGGUCU CCAGUGCA | 6160 |
| 2081 | UGGGCCAG G UCUUGAAG | 2384 | CUUCAAGA GCCGAAAGGCGAGUGAGGUCU CUGGCCCA | 6161 |
| 2090 | UCUUGAAG G CUGUCCAA | 2385 | UUGGACAG GCCGAAAGGCGAGUGAGGUCU CUUCAAGA | 6162 |
| 2093 | UGAAGGCU G UCCAACGA | 2386 | UCGUUGGA GCCGAAAGGCGAGUGAGGUCU AGCCUUCA | 6163 |
| 2106 | ACGAAUGG G CCUAAGAU | 2387 | AUCUUAGG GCCGAAAGGCGAGUGAGGUCU CCAUUCGU | 6164 |
| 2118 | AAGAUCCC G UCCAUCGC | 2388 | GCGAUGGA GCCGAAAGGCGAGUGAGGUCU GGGAUCUU | 6165 |
| 2125 | CGUCCAUC G CCACUGGG | 2389 | CCCAGUGG GCCGAAAGGCGAGUGAGGUCU GAUGGACG | 6166 |
| 2137 | CUGGGAUG G UGGGGGCC | 2390 | GGCCCCCA GCCGAAAGGCGAGUGAGGUCU CAUCCCAG | 6167 |
| 2143 | UGGUGGGG G CCCUCCUC | 2391 | GAGGAGGG GCCGAAAGGCGAGUGAGGUCU CCCCACCA | 6168 |
| 2154 | CUCCUCUU G CUGCUGGU | 2392 | ACCAGCAG GCCGAAAGGCGAGUGAGGUCU AAGAGGAG | 6169 |
| 2157 | CUCUUGCU G CUGGUGGU | 2393 | ACCACCAG GCCGAAAGGCGAGUGAGGUCU AGCAAGAG | 6170 |
| 2161 | UGCUGCUG G UGGUGGCC | 2394 | GGCCACCA GCCGAAAGGCGAGUGAGGUCU CAGCAGCA | 6171 |
| 2164 | UGCUGGUG G UGGCCCUG | 2395 | CAGGGCCA GCCGAAAGGCGAGUGAGGUCU CACCAGCA | 6172 |
| 2167 | UGGUGGUG G CCCUGGGG | 2396 | CCCCAGGG GCCGAAAGGCGAGUGAGGUCU CACCACCA | 6173 |
| 2180 | GGGGAUCG G CCUCUUCA | 2397 | UGAAGAGG GCCGAAAGGCGAGUGAGGUCU CGAUCCCC | 6174 |
| 2190 | CUCUUCAU G CGAAGGCG | 2398 | CGCCUUCG GCCGAAAGGCGAGUGAGGUCU AUGAAGAG | 6175 |
| 2196 | AUGCGAAG G CGCCACAU | 2399 | AUGUGGCG GCCGAAAGGCGAGUGAGGUCU CUUCGCAU | 6176 |
| 2198 | GCGAAGGC G CCACAUCG | 2400 | CGAUGUGG GCCGAAAGGCGAGUGAGGUCU GCCUUCGC | 6177 |
| 2206 | GCCACAUC G UUCGGAAG | 2401 | CUUCCGAA GCCGAAAGGCGAGUGAGGUCU GAUGUGGC | 6178 |
| 2214 | GUUCGGAA G CGCACGCU | 2402 | AGCGUGCG GCCGAAAGGCGAGUGAGGUCU UUCCGAAC | 6179 |
| 2216 | UCGGAAGC G CACGCUGC | 2403 | GCAGCGUG GCCGAAAGGCGAGUGAGGUCU GCUUCCGA | 6180 |
| 2220 | AAGCGCAC G CUGCGGAG | 2404 | CUCCGCAG GCCGAAAGGCGAGUGAGGUCU GUGCGCUU | 6181 |
| 2223 | CGCACGCU G CGGAGGCU | 2405 | AGCCUCCG GCCGAAAGGCGAGUGAGGUCU AGCGUGCG | 6182 |
| 2229 | CUGCGGAG G CUGCUGCA | 2406 | UGCAGCAG GCCGAAAGGCGAGUGAGGUCU CUCCGCAG | 6183 |
| 2232 | CGGAGGCU G CUGCAGGA | 2407 | UCCUGCAG GCCGAAAGGCGAGUGAGGUCU AGCCUCCG | 6184 |
| 2235 | AGGCUGCU G CAGGAGAG | 2408 | CUCUCCUG GCCGAAAGGCGAGUGAGGUCU AGCAGCCU | 6185 |
| 2247 | GAGAGGGA G CUUGUGGA | 2409 | UCCACAAG GCCGAAAGGCGAGUGAGGUCU UCCCUCUC | 6186 |
| 2251 | GGGAGCUU G UGGAGCCU | 2410 | AGGCUCCA GCCGAAAGGCGAGUGAGGUCU AAGCUCCC | 6187 |
| 2256 | CUUGUGGA G CCUCUUAC | 2411 | GUAAGAGG GCCGAAAGGCGAGUGAGGUCU UCCACAAG | 6188 |
| 2270 | UACACCCA G UGGAGAAG | 2412 | CUUCUCCA GCCGAAAGGCGAGUGAGGUCU UGGGUGUA | 6189 |
| 2278 | GUGGAGAA G CUCCCAAC | 2413 | GUUGGGAG GCCGAAAGGCGAGUGAGGUCU UUCUCCAC | 6190 |
| 2290 | CCAACCAA G CUCUCUUG | 2414 | CAAGAGAG GCCGAAAGGCGAGUGAGGUCU UUGGUUGG | 6191 |
| 2335 | AGAUCAAA G UGCUGGGC | 2415 | GCCCAGCA GCCGAAAGGCGAGUGAGGUCU UUUGAUCU | 6192 |
| 2337 | AUCAAAGU G CUGGGCUC | 2416 | GAGCCCAG GCCGAAAGGCGAGUGAGGUCU ACUUUGAU | 6193 |
| 2342 | AGUGCUGG G CUCCGGUG | 2417 | CACCGGAG GCCGAAAGGCGAGUGAGGUCU CCAGCACU | 6194 |
| 2348 | GGGCUCCG G UGCGUUCG | 2418 | CGAACGCA GCCGAAAGGCGAGUGAGGUCU CGGAGCCC | 6195 |
| 2350 | GCUCCGGU G CGUUCGGC | 2419 | GCCGAACG GCCGAAAGGCGAGUGAGGUCU ACCGGAGC | 6196 |
| 2352 | UCCGGUGC G UUCGGCAC | 2420 | GUGCCGAA GCCGAAAGGCGAGUGAGGUCU GCACCGGA | 6197 |
| 2357 | UGCGUUCG G CACGGUGU | 2421 | ACACCGUG GCCGAAAGGCGAGUGAGGUCU CGAACGCA | 6198 |
| 2362 | UCGGCACG G UGUAUAAG | 2422 | CUUAUACA GCCGAAAGGCGAGUGAGGUCU CGUGCCGA | 6199 |
| 2364 | GGCACGGU G UAUAAGGG | 2423 | CCCUUAUA GCCGAAAGGCGAGUGAGGUCU ACCGUGCC | 6200 |
| 2390 | CCCAGAAG G UGAGAAAG | 2424 | CUUUCUCA GCCGAAAGGCGAGUGAGGUCU CUUCUGGG | 6201 |
| 2398 | GUGAGAAA G UUAAAAUU | 2425 | AAUUUUAA GCCGAAAGGCGAGUGAGGUCU UUUCUCAC | 6202 |
| 2410 | AAAUUCCC G UCGCUAUC | 2426 | GAUAGCGA GCCGAAAGGCGAGUGAGGUCU GGGAAUUU | 6203 |
| 2413 | UUCCCGUC G CUAUCAAG | 2427 | CUUGAUAG GCCGAAAGGCGAGUGAGGUCU GACGGGAA | 6204 |
| 2434 | UAAGAGAA G CAACAUCU | 2428 | AGAUGUUG GCCGAAAGGCGAGUGAGGUCU UUCUCUUA | 6205 |
| 2449 | CUCCGAAA G CCAACAAG | 2429 | CUUGUUGG GCCGAAAGGCGAGUGAGGUCU UUUCGGAG | 6206 |
| 2473 | UCGAUGAA G CCUACGUG | 2430 | CACGUAGG GCCGAAAGGCGAGUGAGGUCU UUCAUCGA | 6207 |
| 2479 | AAGCCUAC G UGAUGGCC | 2431 | GGCCAUCA GCCGAAAGGCGAGUGAGGUCU GUAGGCUU | 6208 |
| 2485 | ACGUGAUG G CCAGCGUG | 2432 | CACGCUGG GCCGAAAGGCGAGUGAGGUCU CAUCACGU | 6209 |
| 2489 | GAUGGCCA G CGUGGACA | 2433 | UGUCCACG GCCGAAAGGCGAGUGAGGUCU UGGCCAUC | 6210 |
| 2491 | UGGCCAGC G UGGACAAC | 2434 | GUUGUCCA GCCGAAAGGCGAGUGAGGUCU GCUGGCCA | 6211 |
| 2506 | ACCCCCAC G UGUGCCGC | 2435 | GCGGCACA GCCGAAAGGCGAGUGAGGUCU GUGGGGGU | 6212 |
| 2508 | CCCCACGU G UGCCGCCU | 2436 | AGGCGGCA GCCGAAAGGCGAGUGAGGUCU ACGUGGGG | 6213 |
| 2510 | CCACGUGU G CCGCCUGC | 2437 | GCAGGCGG GCCGAAAGGCGAGUGAGGUCU ACACGUGG | 6214 |
| 2513 | CGUGUGCC G CCUGCUGG | 2438 | CCAGCAGG GCCGAAAGGCGAGUGAGGUCU GGCACACG | 6215 |
| 2517 | UGCCGCCU G CUGGGCAU | 2439 | AUGCCCAG GCCGAAAGGCGAGUGAGGUCU AGGCGGCA | 6216 |
| 2522 | CCUGCUGG G CAUCUGCC | 2440 | GGCAGAUG GCCGAAAGGCGAGUGAGGUCU CCAGCAGG | 6217 |
| 2528 | GGGCAUCU G CCUCACCU | 2441 | AGGUGAGG GCCGAAAGGCGAGUGAGGUCU AGAUGCCC | 6218 |
| 2542 | CCUCCACC G UGCAACUC | 2442 | GAGUUGCA GCCGAAAGGCGAGUGAGGUCU GGUGGAGG | 6219 |
| 2544 | UCCACCGU G CAACUCAU | 2443 | AUGAGUUG GCCGAAAGGCGAGUGAGGUCU ACGGUGGA | 6220 |
| 2556 | CUCAUCAC G CAGCUCAU | 2444 | AUGAGCUG GCCGAAAGGCGAGUGAGGUCU GUGAUGAG | 6221 |
| 2559 | AUCACGCA G CUCAUGCC | 2445 | GGCAUGAG GCCGAAAGGCGAGUGAGGUCU UGCGUGAU | 6222 |
| 2565 | CAGCUCAU G CCCUUCGG | 2446 | CCGAAGGG GCCGAAAGGCGAGUGAGGUCU AUGAGCUG | 6223 |
| 2573 | GCCCUUCG G CUGCCUCC | 2447 | GGAGGCAG GCCGAAAGGCGAGUGAGGUCU CGAAGGGC | 6224 |
| 2576 | CUUCGGCU G CCUCCUGG | 2448 | CCAGGAGG GCCGAAAGGCGAGUGAGGUCU AGCCGAAG | 6225 |
| 2590 | UGGACUAU G UCCGGGAA | 2449 | UUCCCGGA GCCGAAAGGCGAGUGAGGUCU AUAGUCCA | 6226 |
| 2615 | CAAUAUUG G CUCCCAGU | 2450 | ACUGGGAG GCCGAAAGGCGAGUGAGGUCU CAAUAUUG | 6227 |
| 2622 | GGCUCCCA G UACCUGUU | 2451 | AGCAGGUA GCCGAAAGGCGAGUGAGGUCU UGGGAGCC | 6228 |
| 2628 | CAGUACCU G CUCAACUG | 2452 | CAGUUGAG GCCGAAAGGCGAGUGAGGUCU AGGUACUG | 6229 |
| 2637 | CUCAACUG G UGUGUGCA | 2453 | UCCACACA GCCGAAAGGCGAGUGAGGUCU CAGUUGAG | 6230 |
| 2639 | CAACUGGU G UGUGCAGA | 2454 | UCUCCACA GCCGAAAGGCGAGUGAGGUCU ACCAGUUG | 6231 |
| 2641 | ACUGGUGU G UGCAGAUC | 2455 | GAUCUCCA GCCGAAAGGCGAGUGAGGUCU ACACCAGU | 6232 |
| 2643 | UGGUGUGU G CAGAUCGC | 2456 | GCGAUCUG GCCGAAAGGCGAGUGAGGUCU ACACACCA | 6233 |
| 2650 | UCCAGAUC G CAAAGGGC | 2457 | GCCCUUUG GCCGAAAGGCGAGUGAGGUCU GAUCUGCA | 6234 |
| 2657 | CGCAAAGG G CAUGAACU | 2458 | AGUUCAUG GCCGAAAGGCGAGUGAGGUCU CCUUUGCG | 6235 |
| 2678 | GGAGGACC G UCGCUUGG | 2459 | CCAAGCGA GCCGAAAGGCGAGUGAGGUCU GGUCCUCC | 6236 |
| 2681 | GGACCGUC G CUUGGUGC | 2460 | GCACCAAG GCCGAAAGGCGAGUGAGGUCU GACGGUCC | 6237 |
| 2686 | GUCGCUUG G UGCACCGC | 2461 | GCGGUGCA GCCGAAAGGCGAGUGAGGUCU CAAGCGAC | 6238 |
| 2688 | CGCUUGGU G CACCGCGA | 2462 | UCGCGGUG GCCGAAAGGCGAGUGAGGUCU ACCAAGCG | 6239 |
| 2693 | GGUGCACC G CGACCUGG | 2463 | CCAGGUCG GCCGAAAGGCGAGUGAGGUCU GGUGCACC | 6240 |
| 2701 | GCGACCUG G CAGCCAGG | 2464 | CCUGGCUG GCCGAAAGGCGAGUGAGGUCU CAGGUCGC | 6241 |
| 2704 | ACCUGGCA G CCAGGAAC | 2465 | GUUCCUGG GCCGAAAGGCGAGUGAGGUCU UGCCAGGU | 6242 |
| 2713 | CCAGGAAC G UACUGGUG | 2466 | CACCAGUA GCCGAAAGGCGAGUGAGGUCU GUUCCUGG | 6243 |
| 2719 | ACGUACUG G UGAAAACA | 2467 | UGUUUUCA GCCGAAAGGCGAGUGAGGUCU CAGUACGU | 6244 |
| 2730 | AAAACACC G CAGCAUGU | 2468 | ACAUGCUG GCCGAAAGGCGAGUGAGGUCU GGUGUUUU | 6245 |
| 2733 | ACACCGCA G CAUGUCAA | 2469 | UUGACAUG GCCGAAAGGCGAGUGAGGUCU UGCGGUGU | 6246 |
| 2737 | CGCAGCAU G UCAAGAUC | 2470 | GAUCUUGA GCCGAAAGGCGAGUGAGGUCU AUGCUGCG | 6247 |
| 2757 | GAUUUUGG G CUGGCCAA | 2471 | UUGGCCAG GCCGAAAGGCGAGUGAGGUCU CCAAAAUC | 6248 |
| 2761 | UUGGGCUG G CCAAACUG | 2472 | CAGUUUGG GCCGAAAGGCGAGUGAGGUCU CAGCCCAA | 6249 |
| 2769 | GCCAAACU G CUGGGUGC | 2473 | GCACCCAG GCCGAAAGGCGAGUGAGGUCU AGUUUGGC | 6250 |
| 2774 | ACUGCUGG G UGCGGAAG | 2474 | CUUCCGCA GCCGAAAGGCGAGUGAGGUCU CCAGCAGU | 6251 |
| 2776 | UGCUGGGU G CGGAAGAG | 2475 | CUCUUCCG GCCGAAAGGCGAGUGAGGUCU ACCCAGCA | 6252 |
| 2797 | AAUACCAU G CAGAAGGA | 2476 | UCCUUCUG GCCGAAAGGCGAGUGAGGUCU AUGGUAUU | 6253 |
| 2807 | AGAAGGAG G CAAAGUGC | 2477 | GCACUUUG GCCGAAAGGCGAGUGAGGUCU CUCCUUCU | 6254 |
| 2812 | GAGGCAAA G UGCCUAUC | 2478 | GAUAGGCA GCCGAAAGGCGAGUGAGGUCU UUUGCCUC | 6255 |
| 2814 | GGCAAAGU G CCUAUCAA | 2479 | UUGAUAGG GCCGAAAGGCGAGUGAGGUCU ACUUUGCC | 6256 |
| 2823 | CCUAUCAA G UGGAUGGC | 2480 | GCCAUCCA GCCGAAAGGCGAGUGAGGUCU UUGAUAGG | 6257 |
| 2830 | AGUGGAUG G CAUUGGAA | 2481 | UUCCAAUG GCCGAAAGGCGAGUGAGGUCU CAUCCACU | 6258 |
| 2870 | CCACCAGA G UGAUGUCU | 2482 | AGACAUCA GCCGAAAGGCGAGUGAGGUCU UCUGGUGG | 6259 |
| 2875 | AGAGUGAU G UCUGGAGC | 2483 | GCUCCAGA GCCGAAAGGCGAGUGAGGUCU AUCACUCU | 6260 |
| 2882 | UGUCUGGA G CUACGGGG | 2484 | CCCCGUAG GCCGAAAGGCGAGUGAGGUCU UCCAGACA | 6261 |
| 2890 | GCUACGGG G UGACCGUU | 2485 | AACGGUCA GCCGAAAGGCGAGUGAGGUCU CCCGUAGC | 6262 |
| 2896 | GGGUGACC G UUUGGGAG | 2486 | CUCCCAAA GCCGAAAGGCGAGUGAGGUCU GGUCACCC | 6263 |
| 2904 | GUUUGGGA G UUGAUGAC | 2487 | GUCAUCAA GCCGAAAGGCGAGUGAGGUCU UCCCAAAC | 6264 |
| 2925 | GGAUCCAA G CCAUAUGA | 2488 | UCAUAUGG GCCGAAAGGCGAGUGAGGUCU UUGGAUCC | 6265 |
| 2944 | GAAUCCCU G CCAGCGAG | 2489 | CUCGCUGG GCCGAAAGGCGAGUGAGGUCU AGGGAUUC | 6266 |
| 2948 | CCCUGCCA G CGAGAUCU | 2490 | AGAUCUCG GCCGAAAGGCGAGUGAGGUCU UGGCAGGG | 6267 |
| 2981 | AGGAGAAC G CCUCCCUC | 2491 | GAGGGAGG GCCGAAAGGCGAGUGAGGUCU GUUCUCCU | 6268 |
| 2991 | CUCCCUCA G CCACCCAU | 2492 | AUGGGUGG GCCGAAAGGCGAGUGAGGUCU UGAGGGAG | 6269 |
| 3002 | ACCCAUAU G UACCAUCG | 2493 | CGAUGGUA GCCGAAAGGCGAGUGAGGUCU AUAUGGGU | 6270 |
| 3013 | CCAUCGAU G UCUACAUG | 2494 | CAUGUAGA GCCGAAAGGCGAGUGAGGUCU AUCGAUGG | 6271 |
| 3028 | UGAUCAUG G UCAAGUGC | 2495 | GCACUUGA GCCGAAAGGCGAGUGAGGUCU CAUGAUCA | 6272 |
| 3033 | AUGGUCAA G UGCUGGAU | 2496 | AUCCAGCA GCCGAAAGGCGAGUGAGGUCU UUGACCAU | 6273 |
| 3035 | GGUCAAGU G CUGGAUGA | 2497 | UCAUCCAG GCCGAAAGGCGAGUGAGGUCU ACUUGACC | 6274 |
| 3049 | UGAUAGAC G CAGAUAGU | 2498 | ACUAUCUG GCCGAAAGGCGAGUGAGGUCU GUCUAUCA | 6275 |
| 3056 | CGCAGAUA G UCGCCCAA | 2499 | UUGGGCGA GCCGAAAGGCGAGUGAGGUCU UAUCUGCG | 6276 |
| 3059 | AGAUAGUC G CCCAAAGU | 2500 | ACUUUGGG GCCGAAAGGCGAGUGAGGUCU GACUAUCU | 6277 |
| 3066 | CGCCCAAA G UUCCGUGA | 2501 | UCACGGAA GCCGAAAGGCGAGUGAGGUCU UUUGGGCG | 6278 |
| 3071 | AAAGUUCC G UGAGUUGA | 2502 | UCAACUCA GCCGAAAGGCGAGUGAGGUCU GGAACUUU | 6279 |
| 3075 | UUCCGUGA G UUGAUCAU | 2503 | AUGAUCAA GCCGAAAGGCGAGUGAGGUCU UCACGGAA | 6280 |
| 3100 | CCAAAAUG G CCCGAGAC | 2504 | GUCUCGGG GCCGAAAGGCGAGUGAGGUCU CAUUUUGG | 6281 |
| 3114 | GACCCCCA G CGCUACCU | 2505 | AGGUAGCG GCCGAAAGGCGAGUGAGGUCU UGGGGGUC | 6282 |
| 3116 | CCCCCAGC G CUACCUUG | 2506 | CAAGGUAG GCCGAAAGGCGAGUGAGGUCU GCUGGGGG | 6283 |
| 3124 | GCUACCUU G UCAUUCAG | 2507 | CUGAAUGA GCCGAAAGGCGAGUGAGGUCU AAGGUAGC | 6284 |
| 3147 | GAAAGAAU G CAUUUGCC | 2508 | GGCAAAUG GCCGAAAGGCGAGUGAGGUCU AUUCUUUC | 6285 |
| 3153 | AUGCAUUU G CCAAGUCC | 2509 | GGACUUGG GCCGAAAGGCGAGUGAGGUCU AAAUGCAU | 6286 |
| 3158 | UUUGCCAA G UCCUACAG | 2510 | CUGUAGGA GCCGAAAGGCGAGUGAGGUCU UUGGCAAA | 6287 |
| 3182 | CUUCUACC G UGCCCUGA | 2511 | UCAGGGCA GCCGAAAGGCGAGUGAGGUCU GGUAGAAG | 6288 |
| 3184 | UCUACCGU G CCCUGAUG | 2512 | CAUCAGGG GCCGAAAGGCGAGUGAGGUCU ACGGUAGA | 6289 |
| 3214 | UGGACGAC G UGGUGGAU | 2513 | AUCCACCA GCCGAAAGGCGAGUGAGGUCU GUCGUCCA | 6290 |
| 3217 | ACGACGUG G UGGAUGCC | 2514 | GGCAUCCA GCCGAAAGGCGAGUGAGGUCU CACGUCGU | 6291 |
| 3223 | UGGUGGAU G CCGACGAG | 2515 | CUCGUCGG GCCGAAAGGCGAGUGAGGUCU AUCCACCA | 6292 |
| 3231 | GCCGACGA G UACCUCAU | 2516 | AUGAGGUA GCCGAAAGGCGAGUGAGGUCU UCGUCGGC | 6293 |
| 3246 | AUCCCACA G CAGGGCUU | 2517 | AAGCCCUG GCCGAAAGGCGAGUGAGGUCU UGUGGGAU | 6294 |
| 3251 | ACAGCAGG G CUUCUUCA | 2518 | UGAAGAAG GCCGAAAGGCGAGUGAGGUCU CCUGCUGU | 6295 |
| 3260 | CUUCUUCA G CAGCCCCU | 2519 | AGGGGCUG GCCGAAAGGCGAGUGAGGUCU UGAAGAAG | 6296 |
| 3263 | CUUCAGCA G CCCCUCCA | 2520 | UGGAGGGG GCCGAAAGGCGAGUGAGGUCU UGCUGAAG | 6297 |
| 3273 | CCCUCCAC G UCACGGAC | 2521 | GUCCGUGA GCCGAAAGGCGAGUGAGGUCU GUGGAGGG | 6298 |
| 3293 | CCUCCUGA G CUCUCUGA | 2522 | UCAGAGAG GCCGAAAGGCGAGUGAGGUCU UCAGGAGG | 6299 |
| 3302 | CUCUCUGA G UGCAACCA | 2523 | UGGUUGCA GCCGAAAGGCGAGUGAGGUCU UCAGAGAG | 6300 |
| 3304 | CUCUGAGU G CAACCAGC | 2524 | GUUGGUUG GCCGAAAGGCGAGUGAGGUCU ACUCAGAG | 6301 |
| 3311 | UGCAACCA G CAACAAUU | 2525 | AAUUGUUG GCCGAAAGGCGAGUGAGGUCU UGGUUGCA | 6302 |
| 3325 | AUUCCACC G UGGCUUGC | 2526 | GCAAGCCA GCCGAAAGGCGAGUGAGGUCU GGUGGAAU | 6303 |
| 3328 | CCACCGUG G CUUGCAUU | 2527 | AAUGCAAG GCCGAAAGGCGAGUGAGGUCU CACGGUGG | 6304 |
| 3332 | CGUGGCUU G CAUUGAUA | 2528 | UAUCAAUG GCCGAAAGGCGAGUGAGGUCU AAGCCACG | 6305 |
| 3348 | AGAAAUGG G CUGCAAAG | 2529 | CUUUGCAG GCCGAAAGGCGAGUGAGGUCU CCAUUUCU | 6306 |
| 3351 | AAUGGGCU G CAAAGCUG | 2530 | CAGCUUUG GCCGAAAGGCGAGUGAGGUCU AGCCCAUU | 6307 |
| 3356 | GCUGCAAA G CUGUCCCA | 2531 | UGGGACAG GCCGAAAGGCGAGUGAGGUCU UUUGCAGC | 6308 |
| 3359 | GCAAAGCU G UCCCAUCA | 2532 | UGAUGGGA GCCGAAAGGCGAGUGAGGUCU AGCUUUGC | 6309 |
| 3377 | CGAAGACA G CUUCUUGC | 2533 | GCAAGAAG GCCGAAAGGCCACUGAGGUCU UGUCUUCC | 6310 |
| 3384 | AGCUUUCU G CAGCGAUA | 2534 | UAUCGCUG GCCGAAAGGCGAGUGAGGUCU AAGAAGCU | 6311 |
| 3387 | UUCUUGCA G CGAUACAG | 2535 | CUGUAUCG GCCGAAAGGCGAGUGAGGUCU UGCAAGAA | 6312 |
| 3395 | GCGAUACA G CUCAGACC | 2536 | GGUCUGAG GCCGAAAGGCGAGUGAGGUCU UGUAUCGC | 6313 |
| 3410 | CCCCACAG G CGCCUUGA | 2537 | UCAAGGCG GCCGAAAGGCGAGUGAGGUCU CUGUGGGG | 6314 |
| 3412 | CCACAGGC G CCUUGACU | 2538 | AGUCAAGG GCCGAAAGGCGAGUGAGGUCU GCCUGUGG | 6315 |
| 3428 | UGAGGACA G CAUAGACG | 2539 | CGUCUAUG GCCGAAAGGCGAGUGAGGUCU UGUCCUCA | 6316 |
| 3451 | UCCUCCCA G UGCCUGAA | 2540 | UUCAGGCA GCCGAAAGGCGAGUGAGGUCU UGGGAGGA | 6317 |
| 3453 | CUCCCAGU G CCUGAAUA | 2541 | UAUUCAGG GCCGAAAGGCGAGUGAGGUCU ACUGGGAG | 6318 |
| 3471 | AUAAACCA G UCCGUUCC | 2542 | GGAACGGA GCCGAAAGGCGAGUGAGGUCU UGGUUUAU | 6319 |
| 3475 | ACCAGUCC G UUCCCAAA | 2543 | UUUGGGAA GCCGAAAGGCGAGUGAGGUCU GGACUGGU | 6320 |
| 3486 | CCCAAAAG G CCCGCUGG | 2544 | CCAGCGGG GCCGAAAGGCGAGUGAGGUCU CUUUUGGG | 6321 |
| 3490 | AAAGGCCC G CUGGCUCU | 2545 | AGAGCCAG GCCGAAAGGCGAGUGAGGUCU GGGCCUUU | 6322 |
| 3494 | GCCCGCUG G CUCUGUGC | 2546 | GCACAGAG GCCGAAAGGCGAGUGAGGUCU CAGCGGGC | 6323 |
| 3499 | CUCGCUCU G UGCAGAAU | 2547 | AUUCUGCA GCCGAAAGGCGAGUGAGGUCU AGAGCCAG | 6324 |
| 3501 | GCCUCUGU G CAGAAUCC | 2548 | GGAUUCUG GCCCAAAGCCGAGUGAGGUCU ACAGAGCC | 6325 |
| 3511 | AGAAUCCU G UCUAUCAC | 2549 | GUGAUAGA GCCGAAAGGCGAGUGAGGUCU AGCAUUCU | 6326 |
| 3525 | CACAAUCA G CCUCUGAA | 2550 | UUCACAGC CCCGAAACGCGACUGAGGUCU UGAUUGUC | 6327 |
| 3538 | UGAACCCC G CGCCCAGC | 2551 | GCUGCGCC GCCGAAAGGCGAGUGAGGUCU CCGCUUCA | 6328 |
| 3540 | AACCCCGC G CCCACCAC | 2552 | CUCCUCGG GCCGAAAGCCCACUCAGGUCU GCGGCCUU | 6329 |
| 3545 | CGCGCCCA G CAGAGACC | 2553 | CGUCUCUC GCCGAAACGCCAGUCAGGUCU UGGGCGCC | 6330 |
| 3575 | CCCCCACA G CACUCCAC | 2554 | CUCCAGUC GCCGAAAGGCGAGUCAGGUCU UGUGGGGC | 6331 |
| 3580 | ACAGCACU G CACUCCCC | 2555 | GCCCACUC GCCGAAACGCCAGUGAGGUCU ACUCCUCU | 6332 |
| 3583 | GCACUGCA G UGGCCAAC | 2556 | GUUGCCCA GCCGAAAGGCGAGUCAGGUCU UCCACUCC | 6333 |
| 3587 | UCCAGUGC G CAACCCCG | 2557 | CGGCGUUC GCCGAAAGGCGAGUGAGGUCU CCACUGCA | 6334 |
| 3597 | AACCCCGA G UAUCUCAA | 2558 | UUGACAUA GCCGAAAGGCGACUGAGGUCU UCGGCCUU | 6335 |
| 3610 | UCAACACU G UCCAGCCC | 2559 | CGCCUGGA GCCCAAAGCCGAGUGAGGUCU AGUGUUGA | 6336 |
| 3615 | ACUGUCCA G CCCACCUG | 2560 | CACCUGGG GCCGAAAGGCGAGUGAGGUCU UGCACACU | 6337 |
| 3623 | GCCCACCU G UGUCAACA | 2561 | UCUUCACA GCCCAAAGCCGAGUCAGGUCU ACGUCGCC | 6338 |
| 3625 | CCACCUGU G UCAACACC | 2562 | CCUGUUCA GCCCAAAGCCGAGUCAGGUCU ACAGCUCC | 6339 |
| 3632 | UGUCAACA G CACAUUCG | 2563 | CGAAUGUC GCCCAAACGCGAGUGAGGUCU UGUUGACA | 6340 |
| 3644 | AUUCCACA G CCCUGCCC | 2564 | GCGCAGGG GCCCAAAGCCCACUGAGGUCU UGUCGAAU | 6341 |
| 3649 | ACAGCCCU G CCCACUGG | 2565 | CCAGUCGC GCCGAAAGGCGACUGAGGUCU ACCGCUCU | 6342 |
| 3658 | CCCACUCG G CCCAGAAA | 2566 | UUUCUGCG GCCGAAAGGCGAGUCAGGUCU CCACUGCG | 6343 |
| 3668 | CCACAAAC G CACCCACC | 2567 | CCUCCCUC GCCCAAACCCCACUGAGGUCU CUUUCUCC | 6344 |
| 3671 | CAAACCCA G CCACCAAA | 2568 | UUUCCUCC GCCCAAACCCCACUCAGGUCU UCCCUUUC | 6345 |
| 3683 | CCAAAUUA G CCUCCACA | 2569 | UCUCCACC GCCCAAACCCCACUCAGGUCU UAAUUUCC | 6346 |
| 3705 | CACUACCA G CAGCACUU | 2570 | AACUCCUC GCCCAAACCCCACUCAGGUCU UCCUACUC | 6347 |
| 3727 | CCAACCAA G CCAACCCA | 2571 | UCCCUUCC GCCCAAACCCCACUCAGGUCU UUCCUUCC | 6348 |
| 3732 | CAACCCAA G CCAAAUCC | 2572 | CCAUUUCC GCCCAAACCCCACUCAGGUCU UUCCCUUC | 6349 |
| 3740 | CCCAAAUC G CAUCUUUA | 2573 | UAAACAUC GCCCAAACCCCACUCAGGUCU CAUUUGGC | 6350 |
| 3752 | CUUUAAGG G CUCCACAG | 2574 | CUGUGGAG GCCGAAAGGCGAGUGAGGUCU CCUUAAAG | 6351 |
| 3760 | GCUCCACA G CUGAAAAU | 2575 | AUUUUCAG GCCGAAAGGCGAGUGAGGUCU UGUGGAGC | 6352 |
| 3769 | CUGAAAAU G CAGAAUAC | 2576 | GUAUUCUG GCCGAAAGGCGAGUGAGGUCU AUUUUCAG | 6353 |
| 3784 | ACCUAAGG G UCGCGCCA | 2577 | UGGCGCGA GCCGAAAGGCGAGUGAGGUCU CCUUAGGU | 6354 |
| 3787 | UAAGGGUC G CGCCACAA | 2578 | UUGUGGCG GCCGAAAGGCGAGUGAGGUCU GACCCUUA | 6355 |
| 3789 | AGGGUCGC G CCACAAAG | 2579 | CUUUGUGG GCCGAAAGGCGAGUGAGGUCU GCGACCCU | 6356 |
| 3797 | GCCACAAA G CAGUGAAU | 2580 | AUUCACUG GCCGAAAGGCGAGUGAGGUCU UUUGUGGC | 6357 |
| 3800 | ACAAAGCA G UGAAUUUA | 2581 | UAAAUUCA GCCGAAAGGCGAGUGAGGUCU UGCUUUGU | 6358 |
| 3814 | UUAUUGGA G CAUGACCA | 2582 | UGGUCAUG GCCGAAAGGCGAGUGAGGUCU UCCAAUAA | 6359 |
| 3832 | GGAGGAUA G UAUGAGCC | 2583 | GGCUCAUA GCCGAAAGGCGAGUGAGGUCU UAUCCUCC | 6360 |
| 3838 | UAGUAUGA G CCCUAAAA | 2584 | UUUUAGGG GCCGAAAGGCGAGUGAGGUCU UCAUACUA | 6361 |
| 3876 | AGGACCAA G CCACAGCA | 2585 | UGCUGUGG GCCGAAAGGCGAGUGAGGUCU UUGGUCCU | 6362 |
| 3882 | AAGCCACA G CAGGUCCU | 2586 | AGGACCUG GCCGAAAGGCGAGUGAGGUCU UGUGGCUU | 6363 |
| 3886 | CACAGCAG G UCCUCCAU | 2587 | AUGGAGGA GCCGAAAGGCGAGUGAGGUCU CUGCUGUG | 6364 |
| 3902 | UCCCAACA G CCAUGCCC | 2588 | GGGCAUGG GCCGAAAGGCGAGUGAGGUCU UGUUGGGA | 6365 |
| 3907 | ACAGCCAU G CCCGCAUU | 2589 | AAUGCGGG GCCGAAAGGCGAGUGAGGUCU AUGGCUGU | 6366 |
| 3911 | CCAUGCCC G CAUUAGCU | 2590 | AGCUAAUG GCCGAAAGGCGAGUGAGGUCU GGGCAUGG | 6367 |
| 3917 | CCGCAUUA G CUCUUAGA | 2591 | UCUAAGAG GCCGAAAGGCGAGUGAGGUCU UAAUGCGG | 6368 |
| 3937 | ACAGACUG G UUUUGCAA | 2592 | UUGCAAAA GCCGAAAGGCGAGUGAGGUCU CAGUCUGU | 6369 |
| 3942 | CUGGUUUU G CAACGUUU | 2593 | AAACGUUG GCCGAAAGGCGAGUGAGGUCU AAAACCAG | 6370 |
| 3947 | UUUGCAAC G UUUACACC | 2594 | GGUGUAAA GCCGAAAGGCGAGUGAGGUCU GUUGCAAA | 6371 |
| 3961 | ACCGACUA G CCAGGAAG | 2595 | CUUCCUGG GCCGAAAGGCGAGUGAGGUCU UAGUCGGU | 6372 |
| 3969 | GCCAGGAA G UACUUCCA | 2596 | UGGAAGUA GCCGAAAGGCGAGUGAGGUCU UUCCUGGC | 6373 |
| 3984 | CACCUCGG G CACAUUUU | 2597 | AAAAUGUG GCCGAAAGGCGAGUGAGGUCU CCGAGGUG | 6374 |
| 3998 | UUUGGGAA G UUGCAUUC | 2598 | GAAUGCAA GCCGAAAGGCGAGUGAGGUCU UUCCCAAA | 6375 |
| 4001 | GGGAAGUU G CAUUCCUU | 2599 | AAGGAAUG GCCGAAAGGCGAGUGAGGUCU AACUUCCC | 6376 |
| 4011 | AUUCCUUU G UCUUCAAA | 2600 | UUUGAAGA GCCGAAAGGCGAGUGAGGUCU AAAGGAAU | 6377 |
| 4022 | UUCAAACU G UGAAGCAU | 2601 | AUGCUUCA GCCGAAAGGCGAGUGAGGUCU AGUUUGAA | 6378 |
| 4027 | ACUGUGAA G CAUUUACA | 2602 | UGUAAAUG GCCGAAAGGCGAGUGAGGUCU UUCACAGU | 6379 |
| 4041 | ACAGAAAC G CAUCCAGC | 2603 | GCUGGAUG GCCGAAAGGCGAGUGAGGUCU GUUUCUGU | 6380 |
| 4048 | CGCAUCCA G CAAGAAUA | 2604 | UAUUCUUG GCCGAAAGGCGAGUGAGGUCU UGGAUGCG | 6381 |
| 4059 | AGAAUAUU G UCCCUUUG | 2605 | CAAAGGGA GCCGAAAGGCGAGUGAGGUCU AAUAUUCU | 6382 |
| 4069 | CCCUUUGA G CAGAAAUU | 2606 | AAUUUCUG GCCGAAAGGCGAGUGAGGUCU UCAAAGGG | 6383 |
| 4092 | UCAAAGAG G UAUAUUUG | 2607 | CAAAUAUA GCCGAAAGGCGAGUGAGGUCU CUCUUUGA | 6384 |
| 4116 | AAAAAAAA G UAUAUGUG | 2608 | CACAUAUA GCCGAAAGGCGAGUGAGGUCU UUUUUUUU | 6385 |
| 4122 | AAGUAUAU G UGAGGAUU | 2609 | AAUCCUCA GCCGAAAGGCGAGUGAGGUCU AUAUACUU | 6386 |
| 4153 | AUCUUGGA G UUUUUCAU | 2610 | AUGAAAAA GCCGAAAGGCGAGUGAGGUCU UCCAAGAU | 6387 |
| 4163 | UUUUCAUU G UCGCUAUU | 2611 | AAUAGCGA GCCGAAAGGCGAGUGAGGUCU AAUGAAAA | 6388 |
| 4166 | UCAUUGUC G CUAUUGAU | 2612 | AUCAAUAG GCCGAAAGGCGAGUGAGGUCU GACAAUGA | 6389 |
| 4189 | UUCAAUGG G CUCUUCCA | 2613 | UCCAACAC GCCCAAAGGCCACUCAGGUCU CCAUUGAA | 6390 |
| 4209 | AGGAAGAA G CUUGCUGG | 2614 | CCAGCAAG GCCGAAAGGCGAGUGAGGUCU UUCUUCCU | 6391 |
| 4213 | AGAAGCUU G CUGGUAGC | 2615 | GCUACCAG GCCGAAAGGCGAGUGAGGUCU AAGCUUCU | 6392 |
| 4217 | GCUUGCUG G UAGCACUU | 2616 | AAGUGCUA GCCGAAAGGCGAGUGAGGUCU CAGCAAGC | 6393 |
| 4220 | UCCUCGUA G CACUUGCU | 2617 | AGCAAGUG GCCGAAAGGCGAGUGAGGUCU UACCAGCA | 6394 |
| 4226 | UAGCACUU G CUACCCUG | 2618 | CAGGGUAG GCCGAAAGGCGAGUGAGGUCU AAGUGCUA | 6395 |
| 4236 | UACCCUCA G UUCAUCCA | 2619 | UCGAUGAA GCCGAAAGCCGAGUGAGGUCU UCAGGGUA | 6396 |
| 4246 | UCAUCCAC G CCCAACUC | 2620 | CAGUUGGG GCCGAAAGGCGAGUGAGGUCU CUGGAUGA | 6397 |
| 4254 | GCCCAACU G UGAGCAAG | 2621 | CUUGCUCA GCCGAAAGGCGAGUGAGGUCU AGUUGGGC | 6398 |
| 4258 | AACUGUGA G CAAGGAGC | 2622 | GCUCCUUG GCCGAAAGGCGAGUGAGGUCU UCACAGUU | 6399 |
| 4265 | AGCAAGGA G CACAAGCC | 2623 | GGCUUCUG GCCGAAAGGCGAGUGAGGUCU UCCUUGCU | 6400 |
| 4271 | GAGCACAA G CCACAAGU | 2624 | ACUUGUGG GCCGAAAGGCGAGUCAGGUCU UUGUGCUC | 6401 |
| 4278 | AGCCACAA G UCUUCCAG | 2625 | CUGGAAGA GCCGAAAGGCGAGUGAGGUCU UUGUGGCU | 6402 |
| 4292 | CAGAGGAU G CUUGAUUC | 2626 | GAAUCAAG GCCGAAAGGCGAGUGAGGUCU AUCCUCUG | 6403 |
| 4303 | UGAUUCCA G UGGUUCUG | 2627 | CAGAACCA GCCGAAAGGCGAGUGAGGUCU UGGAAUCA | 6404 |
| 4306 | UUCCAGUG G UUCUGCUU | 2628 | AAGCAGAA GCCGAAAGGCGAGUGAGGUCU CACUGGAA | 6405 |
| 4311 | GUGGUUCU G CUUCAAGG | 2629 | CCUUGAAG GCCGAAAGGCGAGUGAGGUCU AGAACCAC | 6406 |
| 4319 | GCUUCAAG G CUUCCACU | 2630 | AGUGGAAG GCCGAAAGGCGAGUGAGGUCU CUUGAAGC | 6407 |
| 4328 | CUUCCACU G CAAAACAC | 2631 | GUGUUUUG GCCGAAAGGCGAGUGAGGUCU AGUGGAAG | 6408 |
| 4352 | CCAAGAAG G CCUUCAUG | 2632 | CAUGAAGG GCCGAAAGGCGAGUGAGGUCU CUUCUUGG | 6409 |
| 4361 | CCUUCAUG G CCCCAGCA | 2633 | UGCUGGGG GCCGAAAGGCGAGUGAGGUCU CAUGAAGG | 6410 |
| 4367 | UGGCCCCA G CAGGCCGG | 2634 | CCGGCCUG GCCGAAAGGCGAGUGAGGUCU UGGGGCCA | 6411 |
| 4371 | CCCAGCAG G CCGGAUCG | 2635 | CGAUCCGG GCCGAAAGGCGAGUGAGGUCU CUGCUGGG | 6412 |
| 4380 | CCGGAUCG G UACUGUAU | 2636 | AUACAGUA GCCGAAAGGCGAGUGAGGUCU CGAUCCGG | 6413 |
| 4385 | UCGGUACU G UAUCAAGU | 2637 | ACUUGAUA GCCGAAAGGCGAGUGAGGUCU AGUACCGA | 6414 |
| 4392 | UGUAUCAA G UCAUGGCA | 2638 | UGCCAUGA GCCGAAAGGCGAGUGAGGUCU UUGAUACA | 6415 |
| 4398 | AAGUCAUG G CAGGUACA | 2639 | UGUACCUG GCCGAAAGGCGAGUGAGGUCU CAUGACUU | 6416 |
| 4402 | CAUGGCAG G UACAGUAG | 2640 | CUACUGUA GCCGAAAGGCGAGUGAGGUCU CUGCCAUG | 6417 |
| 4407 | CAGGUACA G UAGGAUAA | 2641 | UUAUCCUA GCCGAAAGGCGAGUGAGGUCU UGUACCUG | 6418 |
| 4416 | UAGGAUAA G CCACUCUG | 2642 | CAGAGUGG GCCGAAAGGCGAGUGAGGUCU UUAUCCUA | 6419 |
| 4424 | GCCACUCU G UCCCUUCC | 2643 | GGAAGGGA GCCGAAAGGCGAGUGAGGUCU AGAGUGGC | 6420 |
| 4436 | CUEUCUGG G CAAAGAAG | 2644 | CUUCUUUG GCCGAAAGGCGAGUGAGGUCU CCAGGAAG | 6421 |
| 4482 | UUACUUUU G UAAAAAUG | 2645 | CAUUUUUA GCCGAAAGGCGAGUGAGGUCU AAAAGUAA | 6422 |
| 4490 | GUAAAAAU G UCCCCACG | 2646 | CGUGGGGA GCCGAAAGGCGAGUGAGGUCU AUUUUUAC | 6423 |
| 4499 | UCCCCACG G UACUUACU | 2647 | AGUAAGUA GCCGAAAGGCGAGUGAGGUCU CGUGGGGA | 6424 |
| 4524 | AUGGACCA G UGGUUUCC | 2648 | GGAAACCA GCCGAAAGGCGAGUGAGGUCU UGGUCCAU | 6425 |
| 4527 | GACCAGUG G UUUCCAGU | 2649 | ACUGGAAA GCCGAAAGGCGAGUGAGGUCU CACUGGUC | 6426 |
| 4534 | GGUUUCCA G UCAUGAGC | 2650 | GCUCAUGA GCCGAAAGGCGAGUGAGGUCU UGGAAACC | 6427 |
| 4541 | AGUCAUGA G CGUUAGAC | 2651 | GUCUAACG GCCGAAAGGCGAGUGAGGUCU UCAUGACU | 6428 |
| 4543 | UCAUGAGC G UUAGACUG | 2652 | CAGUCUAA GCCGAAAGGCGAGUGAGGUCU GCUCAUGA | 6429 |
| 4556 | ACUGACUU G UUUGUCUU | 2653 | AAGACAAA GCCGAAAGGCGAGUGAGGUCU AAGUCAGU | 6430 |
| 4560 | ACUUGUUU G UCUUCCAU | 2654 | AUGGAAGA GCCGAAAGGCGAGUGAGGUCU AAACAAGU | 6431 |
| 4575 | AUUCCAUU G UUUUGAAA | 2655 | UUUCAAAA GCCGAAAGGCGAGUGAGGUCU AAUGGAAU | 6432 |
| 4588 | GAAACUCA G UAUGCCGC | 2656 | GCGGCAUA GCCGAAAGGCGAGUGAGGUCU UGAGUUUC | 6433 |
| 4592 | CUCAGUAU G CCGCCCCU | 2657 | AGGGGCGG GCCGAAAGGCGAGUGAGGUCU AUACUGAG | 6434 |
| 4595 | AGUAUGCC G CCCCUGUC | 2658 | GACAGGGG GCCGAAAGGCGAGUGAGGUCU GGCAUACU | 6435 |
| 4601 | CCGCCCCU G UCUUGCUG | 2659 | CAGCAAGA GCCGAAAGGCGAGUGAGGUCU AGGGGCGG | 6436 |
| 4606 | CCUGUCUU G CUGUCAUG | 2660 | CAUGACAG GCCGAAAGGCGAGUGAGGUCU AAGACAGG | 6437 |
| 4609 | GUCUUGCU G UCAUGAAA | 2661 | UUUCAUGA GCCGAAAGGCGAGUGAGGUCU AGCAAGAC | 6438 |
| 4621 | UGAAAUCA G CAAGAGAG | 2662 | CUCUCUUG GCCGAAAGGCGAGUGAGGUCU UGAUUUCA | 6439 |
| 4661 | GGAUUCCA G CCCACAUU | 2663 | AAUGUGGG GCCGAAAGGCGAGUGAGGUCU UGGAAUCC | 6440 |
| 4680 | AUUCAUCA G CAUUUGGA | 2664 | UCCAAAUG GCCGAAAGGCGAGUGAGGUCU UGAUGAAU | 6441 |
| 4695 | GACCAAUA G CCCACAGC | 2665 | GCUGUGGG GCCGAAAGGCGAGUGAGGUCU UAUUGGUC | 6442 |
| 4702 | AGCCCACA G CUGAGAAU | 2666 | AUUCUCAG GCCGAAAGGCGAGUGAGGUCU UGUGGGCU | 6443 |
| 4711 | CUGAGAAU G UGGAAUAC | 2667 | GUAUUCCA GCCGAAAGGCGAGUGAGGUCU AUUCUCAG | 6444 |
| 4734 | AUAACACC G CUUUUGUU | 2668 | AACAAAAG GCCGAAAGGCGAGUGAGGUCU GGUGUUAU | 6445 |
| 4740 | CCGCUUUU G UUCUCGCA | 2669 | UGCGAGAA GCCGAAAGGCGAGUGAGGUCU AAAAGCGG | 6446 |
| 4748 | UUGUUCUC G CAAAAACG | 2670 | CGUUUUUG GCCGAAAGGCGAGUGAGGUCU GAGAACAA | 6447 |
| 4754 | GCAAAAAC G UAUCUCCU | 2671 | AGGAGAUA GCCGAAAGGCGAGUGAGGUCU GUUUUUGC | 6448 |
| 4771 | AAUUUGAG G CUCAGAUG | 2672 | CAUCUGAG GCCGAAAGGCGAGUGAGGUCU CUCAAAUU | 6449 |
| 4784 | GAUGAAAU G CAUCAGGU | 2673 | ACCUGAUG GCCGAAAGGCGAGUGAGGUCU AUUUCAUC | 6450 |
| 4791 | UGCAUCAG G UCCUUUGG | 2674 | CCAAAGGA GCCGAAAGGCGAGUGAGGUCU CUGAUGCA | 6451 |
| 4801 | CCUUUGGG G CAUAGAUC | 2675 | GAUCUAUG GCCGAAAGGCGAGUGAGGUCU CCCAAAGG | 6452 |
| 4829 | AAAAUGAA G CUGCUCUG | 2676 | CAGAGCAG GCCGAAAGGCGAGUGAGGUCU UUCAUUUU | 6453 |
| 4832 | AUGAAGCU G CUCUGAAA | 2677 | UUUCAGAG GCCGAAAGGCGAGUGAGGUCU AGCUUCAU | 6454 |
| 4850 | CUCCUUUA G CCAUCACC | 2678 | GGUGAUGG GCCGAAAGGCGAGUGAGGUCU UAAAGGAG | 6455 |
| 4876 | CAAAAUUA G UUUGUGUU | 2679 | AACACAAA GCCGAAAGGCGAGUGAGGUCU UAAUUUUG | 6456 |
| 4880 | AUUAGUUU G UGUUACUU | 2680 | AAGUAACA GCCGAAAGGCGAGUGAGGUCU AAACUAAU | 6457 |
| 4882 | UAGUUUGU G UUACUUAU | 2681 | AUAAGUAA GCCGAAAGGCGAGUGAGGUCU ACAAACUA | 6458 |
| 4899 | GGAAGAUA G UUUUCUCC | 2682 | GGAGAAAA GCCGAAAGGCGAGUGAGGUCU UAUCUUCC | 6459 |
| 4926 | CUUCAAAA G CUUUUUAC | 2683 | GUAAAAAG GCCGAAAGGCGAGUGAGGUCU UUUUGAAG | 6460 |
| 4942 | CUCAAAGA G UAUAUGUU | 2684 | AACAUAUA GCCGAAAGGCGAGUGAGGUCU UCUUUGAG | 6461 |
| 4948 | GAGUAUAU G UUCCCUCC | 2685 | GGAGGGAA GCCGAAAGGCGAGUGAGGUCU AUAUACUC | 6462 |
| 4959 | CCCUCCAG G UCAGCUGC | 2686 | GCAGCUGA GCCGAAAGGCGAGUGAGGUCU CUGGAGGG | 6463 |
| 4963 | CCAGGUCA G CUGCCCCC | 2687 | GGGGGCAG GCCGAAAGGCGAGUGAGGUCU UGACCUGG | 6464 |
| 4966 | GGUCAGCU G CCCCCAAA | 2688 | UUUGGGGG GCCGAAAGGCGAGUGAGGUCU AGCUGACC | 6465 |
| 4987 | CUCCUUAC G CUUUGUCA | 2689 | UGACAAAG GCCGAAAGGCGAGUGAGGUCU GUAAGGAG | 6466 |
| 4992 | UACGCUUU G UCACACAA | 2690 | UUGUGUGA GCCGAAAGGCGAGUGAGGUCU AAAGCGUA | 6467 |
| 5004 | CACAAAAA G UGUCUCUG | 2691 | CAGAGACA GCCGAAAGGCGAGUGAGGUCU UUUUUGUG | 6468 |
| 5006 | CAAAAAGU G UCUCUGCC | 2692 | GGCAGAGA GCCGAAAGGCGAGUGAGGUCU ACUUUUUG | 6469 |
| 5012 | GUGUCUCU G CCUUGAGU | 2693 | ACUCAAGG GCCGAAAGGCGAGUGAGGUCU AGAGACAC | 6470 |
| 5019 | UGCCUUGA G UCAUCUAU | 2694 | AUAGAUGA GCCGAAAGGCGAGUGAGGUCU UCAAGGCA | 6471 |
| 5032 | CUAUUCAA G CACUUACA | 2695 | UGUAAGUG GCCGAAAGGCGAGUGAGGUCU UUGAAUAG | 6472 |
| 5041 | CACUUACA G CUCUGGCC | 2696 | GGCCAGAG GCCGAAAGGCGAGUGAGGUCU UGUAAGUG | 6473 |
| 5047 | CAGCUCUG G CCACAACA | 2697 | UGUUGUGG GCCGAAAGGCGAGUGAGGUCU CAGAGCUG | 6474 |
| 5058 | ACAACAGG G CAUUUUAC | 2698 | GUAAAAUG GCCGAAAGGCGAGUGAGGUCU CCUGUUGU | 6475 |
| 5069 | UUUUACAG G UGCGAAUG | 2699 | CAUUCGCA GCCGAAAGGCGAGUGAGGUCU CUGUAAAA | 6476 |
| 5071 | UUACAGGU G CGAAUGAC | 2700 | GUCAUUCG GCCGAAAGGCGAGUGAGGUCU ACCUGUAA | 6477 |
| 5081 | GAAUGACA G UAGCAUUA | 2701 | UAAUGCUA GCCGAAAGGCGAGUGAGGUCU UGUCAUUC | 6478 |
| 5084 | UGACAGUA G CAUUAUGA | 2702 | UCAUAAUG GCCGAAAGGCGAGUGAGGUCU UACUGUCA | 6479 |
| 5093 | CAUUAUGA G UAGUGUGA | 2703 | UCACACUA GCCGAAAGGCGAGUGAGGUCU UCAUAAUG | 6480 |
| 5096 | UAUGAGUA G UGUGAAUU | 2704 | AAUUCACA GCCGAAAGGCGAGUGAGGUCU UACUCAUA | 6481 |
| 5098 | UGAGUAGU G UGAAUUCA | 2705 | UGAAUUCA GCCGAAAGGCGAGUGAGGUCU ACUACUCA | 6482 |
| 5108 | GAAUUCAG G UAGUAAAU | 2706 | AUUUACUA GCCGAAAGGCGAGUGAGGUCU CUGAAUUC | 6483 |
| 5111 | UUCAGGUA G UAAAUAUG | 2707 | CAUAUUUA GCCGAAAGGCGAGUGAGGUCU UACCUGAA | 6484 |
| 5128 | AAACUAGG G UUUGAAAU | 2708 | AUUUCAAA GCCGAAAGGCGAGUGAGGUCU CCUAGUUU | 6485 |
| 5144 | UUGAUAAU G CUUUCACA | 2709 | UGUGAAAG GCCGAAAGGCGAGUGAGGUCU AUUAUCAA | 6486 |
| 5159 | CAACAUUU G CAGAUGUU | 2710 | AACAUCUG GCCGAAAGGCGAGUGAGGUCU AAAUGUUG | 6487 |
| 5165 | UUGCAGAU G UUUUAGAA | 2711 | UUCUAAAA GCCGAAAGGCGAGUGAGGUCU AUCUGCAA | 6488 |
| 5182 | GGAAAAAA G UUCCUUCC | 2712 | GGAAGGAA GCCGAAAGGCGAGUGAGGUCU UUUUUUCC | 6489 |
| 5230 | AAGAUUCA G CUAGUUAG | 2713 | CUAACUAG GCCGAAAGGCGAGUGAGGUCU UGAAUCUU | 6490 |
| 5234 | UUCAGCUA G UUAGGAGC | 2714 | GCUCCUAA GCCGAAAGGCGAGUGAGGUCU UAGCUGAA | 6491 |
| 5241 | AGUUAGGA G CCCAUUUU | 2715 | AAAAUGGG GCCGAAAGGCGAGUGAGGUCU UCCUAACU | 6492 |
| 5260 | CCUAAUCU G UGUGUGCC | 2716 | GGCACACA GCCGAAAGGCGAGUGAGGUCU AGAUUAGG | 6493 |
| 5262 | UAAUCUGU G UGUGCCCU | 2717 | AGGGCACA GCCGAAAGGCGAGUGAGGUCU ACAGAUGA | 6494 |
| 5264 | AUCUGUGU G UGCCCUGU | 2718 | ACAGGGCA GCCGAAAGGCGAGUGAGGUCU ACACAGAU | 6495 |
| 5266 | CUGUGUGU G CCCUGUAA | 2719 | UUACAGGG GCCGAAAGGCGAGUGAGGUCU ACACACAG | 6496 |
| 5271 | UGUGCCCU G UAACCUGA | 2720 | UCAGGUUA GCCGAAAGGCGAGUGAGGUCU AGGGCACA | 6497 |
| 5283 | CCUGACUG G UUAACAGC | 2721 | GCUGUUAA GCCGAAAGGCGAGUGAGGUCU CAGUCAGG | 6498 |
| 5290 | GGUUAACA G CAGUCCUU | 2722 | AAGGACUG GCCGAAAGGCGAGUGAGGUCU UGUUAACC | 6499 |
| 5293 | UAACAGCA G UCCUUUGU | 2723 | ACAAAGGA GCCGAAAGGCGAGUGAGGUCU UCCUGUUA | 6500 |
| 5300 | AGUCCUUU G UAAACAGU | 2724 | ACUGUUUA GCCGAAAGGCGAGUGAGGUCU AAAGGACU | 6501 |
| 5307 | UGUAAACA G UGUUUUAA | 2725 | UUAAAACA GCCGAAAGGCGAGUGAGGUCU UGUUUACA | 6502 |
| 5309 | UAAACAGU G UUUUAAAC | 2726 | GUUUAAAA GCCGAAAGGCGAGUGAGGUCU ACUGUUUA | 6503 |
| 5325 | CUCUCCUA G UCAAUAUC | 2727 | GAUAUUGA GCCGAAAGGCGAGUGAGGUCU UAGGAGAG | 6504 |
| 5364 | AAGAAAUG G UUCAGAAA | 2728 | UUUCUGAA GCCGAAAGGCGAGUGAGGUCU CAUUUCUU | 6505 |
| 5382 | UAUUUUCA G CCUACAGU | 2729 | ACUGUAGG GCCGAAAGGCGAGUGAGGUCU UGAAAAUA | 6506 |
| 5389 | AGCCUACA G UUAUGUUC | 2730 | GAACAUAA GCCGAAAGGCGAGUGAGGUCU UGUAGGCU | 6507 |
| 5394 | ACAGUUAU G UUCAGUCA | 2731 | UGACUGAA GCCGAAAGGCGAGUGAGGUCU AUAACUGU | 6508 |
| 5399 | UAUGUUCA G UCACACAC | 2732 | GUGUGUGA GCCGAAAGGCGAGUGAGGUCU UGAACAUA | 6509 |
| 5419 | UACAAAAU G UUCCUUUU | 2733 | AAAAGGAA GCCGAAAGGCGAGUGAGGUCU AUUUUGUA | 6510 |
| 5428 | UUCCUUUU G CUUUUAAA | 2734 | UUUAAAAG GCCGAAAGGCGAGUGAGGUCU AAAAGGAA | 6511 |
| 5437 | CUUUUAAA G UAAUUUUU | 2735 | AAAAAUUA GCCGAAAGGCGAGUGAGGUCU UUUAAAAG | 6512 |
| 5459 | CCAGAUCA G UCAGAGCC | 2736 | GGCUCUGA GCCGAAAGGCGAGUGAGGUCU UGAUCUGG | 6513 |
| 5465 | CAGUCAGA G CCCCUACA | 2737 | UGUAGGGG GCCGAAAGGCGAGUGAGGUCU UCUGACUG | 6514 |
| 5474 | CCCCUACA G CAUUGUUA | 2738 | UAACAAUG GCCGAAAGGCGAGUGAGGUCU UGUAGGGG | 6515 |
| 5479 | ACAGCAUU G UUAAGAAA | 2739 | UUUCUUAA GCCGAAAGGCGAGUGAGGUCU AAUGCUGU | 6516 |
| 5488 | UUAAGAAA G UAUUUGAU | 2740 | AUCAAAUA GCCGAAAGGCGAGUGAGGUCU UUUCUUAA | 6517 |
| 5501 | UGAUUUUU G UCUCAAUG | 2741 | CAUUGAGA GCCGAAAGGCGAGUGAGGUCU AAAAAUCA | 6518 |

Input Sequence = N_005228.1. Cut Site = G/Y
Arm Length = 8. Core Sequence = GCcgaaagGCGaGuCaaGGuCu
NM_005228.1 (Homo sapiens epidermal growth tactor receptor (avian erythroblastic leukemia viral (v-erb-b) oncogene homolog) (EGFR), mPNA)

TABLE VI
Human EGFR Receptor DNAzyme and Substrate Sequence

| Pos | Substrate | Seq ID | DNAzyme | Seq ID |
|---|---|---|---|---|
| 9 | GCCGCGCU G CGCCGGAG | 2118 | CTCCGGCG GGCTAGCTACAACGA AGCGCGGC | 6519 |
| 11 | CGCGCUGC G CCGGAGUC | 2119 | GACTCCGG GGCTAGCTACAACGA GCAGCGCG | 6520 |
| 17 | GCGCCGGA G UCCCGAGC | 2120 | GCTCGGGA GGCTAGCTACAACGA TCCGGCGC | 6521 |
| 24 | AGUCCCGA G CUAGCCCC | 2121 | GGGGCTAG GGCTAGCTACAACGA TCGGGACT | 6522 |
| 28 | CCGAGCUA G CCCCGGCG | 2122 | CGCCGGGG GGCTAGCTACAACGA TAGCTCGG | 6523 |
| 34 | UAGCCCCG G CGCCGCCG | 2123 | CGGCGGCG GGCTAGCTACAACGA CGGGGCTA | 6524 |
| 36 | GCCCCGGC G CCGCCGCC | 2124 | GGCGGCGG GGCTAGCTACAACGA GCCGGGCC | 6525 |
| 39 | CCGGCGCC G CCGCCGCC | 2125 | GGCGGCGG GGCTAGCTACAACGA GGCGCCGG | 6526 |
| 42 | GCGCCGCC G CCGCCCAG | 2126 | CTGGGCGG GGCTAGCTACAACGA GGCGGCGC | 6527 |
| 45 | CCGCCGCC G CCCAGACC | 2127 | GGTCTGGG GGCTAGCTACAACGA GGCGGCGG | 6528 |
| 51 | CCGCCCAG G CCGGACGA | 2742 | TCGTCCGG GGCTAGCTACAACGA CTGGGCGG | 6529 |
| 56 | CAGACCGG G CGACAGGC | 2743 | GCCTGTCG GGCTAGCTACAACGA CCGGTCTG | 6530 |
| 59 | ACCGGACG G CAGGCCAC | 2744 | GTGGCCTG GGCTAGCTACAACGA CGTCCGGT | 6531 |
| 63 | GACGACAG G CCACCUCG | 2128 | CGAGGTGG GGCTAGCTACAACGA CTGTCGTC | 6532 |
| 66 | GACAGGCC G CCUCGUCG | 840 | CGACGAGG GGCTAGCTACAACGA GGCCTGTC | 6533 |
| 71 | GCCACCUC G UCGGCGUC | 2129 | GACGCCGA GGCTAGCTACAACGA GAGGTGGC | 6534 |
| 75 | CCUCGUCG G CGUCCGCC | 2130 | GGCGGACG GGCTAGCTACAACGA CGACGAGG | 6535 |
| 77 | UCGUCGGC G UCCGCCCG | 2131 | CGGGCGGA GGCTAGCTACAACGA GCCGACGA | 6536 |
| 81 | CGGCGUCC G CCCGAGUC | 2132 | GACTCGGG GGCTAGCTACAACGA GGACGCCG | 6537 |
| 87 | CCGCCCGA G UCCCCGCC | 2133 | GGCGGGGA GGCTAGCTACAACGA TCGGGCGG | 6538 |
| 93 | GAGUCCCC G CCUCGCCG | 2134 | CGGCGAGG GGCTAGCTACAACGA GGGGACTC | 6539 |
| 98 | CCCGCCUC G CCGCCAAC | 2135 | GTTGGCGG GGCTAGCTACAACGA GAGGCGGG | 6540 |
| 101 | GCCUCGCC G CCAACGCC | 2136 | GGCGTTGG GGCTAGCTACAACGA GGCGAGGC | 6541 |
| 105 | CGCCGCCA G CGCCACAA | 2745 | TTGTGGCG GGCTAGCTACAACGA TGGCGGCG | 6542 |
| 107 | CCGCCAAC G CCACAACC | 2137 | GGTTGTGG GGCTAGCTACAACGA GTTGGCGG | 6543 |
| 110 | CCAACGCC G CAACCACC | 855 | GGTGGTTG GGCTAGCTACAACGA GGCGTTGG | 6544 |
| 113 | ACGCCACA G CCACCGCG | 2746 | CGCGGTGG GGCTAGCTACAACGA TGTGGCGT | 6545 |
| 116 | CCACAACC G CCGCGCAC | 858 | GTGCGCGG GGCTAGCTACAACGA GGTTGTGG | 6546 |
| 119 | CAACCACC G CGCACGGC | 2138 | GCCGTGCG GGCTAGCTACAACGA GGTGGTTG | 6547 |
| 121 | ACCACCGC G CACGGCCC | 2139 | GGGCCGTG GGCTAGCTACAACGA GCGGTGGT | 6548 |
| 123 | CACCGCGC G CGGCCCCC | 860 | GGGGGCCG GGCTAGCTACAACGA GCGCGGTG | 6549 |
| 126 | CGCGCACG G CCCCCUGA | 2140 | TCAGGGGG GGCTAGCTACAACGA CGTGCGCG | 6550 |
| 134 | GCCCCCUG G CUCCGUCC | 2747 | GGACGGAG GGCTAGCTACAACGA CAGGGGGC | 6551 |
| 139 | CUGACUCC G UCCAGUAU | 2141 | ATACTGGA GGCTAGCTACAACGA GCAGTCAC | 6552 |
| 144 | UCCGUCCA G UAUUGAUC | 2142 | GATCAATA GGCTAGCTACAACGA TGGACGGA | 6553 |
| 146 | CGUCCAGU G UUGAUCGG | 10 | CCGATCAA GGCTAGCTACAACGA ACTGGACG | 6554 |
| 150 | CAGUAUUG G UCGGGAGA | 2748 | TCTCCCGA GGCTAGCTACAACGA CAATACTG | 6555 |
| 159 | UCGGGAGA G CCGGAGCG | 2143 | CGCTCCGG GGCTAGCTACAACGA TCTCCCGA | 6556 |
| 165 | GAGCCGGA G CGAGCUCU | 2144 | AGAGCTCG GGCTAGCTACAACGA TCCGGCTC | 6557 |
| 169 | CGGAGCGA G CUCUUCGG | 2145 | CCGAAGAG GGCTAGCTACAACGA TCGCTCCG | 6558 |
| 181 | UUCGGGGA G CAGCGAUG | 2146 | CATCGCTG GGCTAGCTACAACGA TCCCCGAA | 6559 |
| 184 | GGGGAGCA G CGAUGCGA | 2147 | TCGCATCG GGCTAGCTACAACGA TGCTCCCC | 6560 |
| 187 | GAGCAGCG G UGCGACCC | 2749 | GGGTCGCA GGCTAGCTACAACGA CGCTGCTC | 6561 |
| 189 | GCAGCGAU G CCACCCUC | 2148 | GAGGGTCG GGCTAGCTACAACGA ATCGCTGC | 6562 |
| 192 | GCGAUGCG G CCCUCCGG | 2750 | CCGGAGGG GGCTAGCTACAACGA CGCATCGC | 6563 |
| 202 | CCUCCGGG G CGGCCGGG | 2751 | CCCGGCCG GGCTAGCTACAACGA CCCGGAGG | 6564 |
| 205 | CCGCGACG G CCGGGGCA | 2149 | TGCCCCGG GGCTAGCTACAACGA CGTCCCGG | 6565 |
| 211 | CGGCCGGG G CAGCGCUC | 2150 | GAGCGCTG GGCTAGCTACAACGA CCCGGCCG | 6566 |
| 214 | CCGGGGCA G CGCUCCUG | 2151 | CAGGAGCG GGCTAGCTACAACGA TGCCCCGG | 6567 |
| 216 | GGGGCAGC G CUCCUGGC | 2152 | GCCAGGAG GGCTAGCTACAACGA GCTGCCCC | 6568 |
| 223 | CGCUCCUG G CGCUGCUG | 2153 | CAGCAGCG GGCTAGCTACAACGA CAGGAGCG | 6569 |
| 225 | CUCCUGGC G CUGCUGGC | 2154 | GCCAGCAG GGCTAGCTACAACGA GCCAGGAG | 6570 |
| 228 | CUGGCGCU G CUGGCUGC | 2155 | GCAGCCAG GGCTAGCTACAACGA AGCGCCAG | 6571 |
| 232 | CGCUGCUG G CUGCGCUC | 2156 | GAGCGCAG GGCTAGCTACAACGA CAGCAGCG | 6572 |
| 235 | UGCUGGCU G CGCUCUGC | 2157 | GCAGAGCG GGCTAGCTACAACGA AGCCAGCA | 6573 |
| 237 | CUGGCUGC G CUCUGCCC | 2158 | GGGCAGAG GGCTAGCTACAACGA GCAGCCAG | 6574 |
| 242 | UGCGCUCU G CCCGGCGA | 2159 | TCGCCGGG GGCTAGCTACAACGA AGAGCGCA | 6575 |
| 247 | UCUGCCCG G CGAGUCGG | 2160 | CCGACTCG GGCTAGCTACAACGA CGGGCAGA | 6576 |
| 251 | CCCGGCGA G UCGGGCUC | 2161 | GAGCCCGA GGCTAGCTACAACGA TCGCCGGG | 6577 |
| 256 | CGAGUCGG G CUCUGGAG | 2162 | CTCCAGAG GGCTAGCTACAACGA CCGACTCG | 6578 |
| 274 | AAAAGAAA G UUUGCCAA | 2163 | TTGGCAAA GGCTAGCTACAACGA TTTCTTTT | 6579 |
| 278 | GAAAGUUU G CCAAGGCA | 2164 | TGCCTTGG GGCTAGCTACAACGA AAACTTTC | 6580 |
| 284 | UUGCCAAG G CACGAGUA | 2165 | TACTCGTG GGCTAGCTACAACGA CTTGGCAA | 6581 |
| 286 | GCCAAGGC G CGAGUAAC | 894 | GTTACTCG GGCTAGCTACAACGA GCCTTGGC | 6582 |
| 290 | AGGCACGA G UAACAAGC | 2166 | GCTTGTTA GGCTAGCTACAACGA TCGTGCCT | 6583 |
| 293 | CACGAGUA G CAAGCUCA | 2752 | TGAGCTTG GGCTAGCTACAACGA TACTCGTG | 6584 |
| 297 | AGUAACAA G CUCACGCA | 2167 | TGCGTGAG GGCTAGCTACAACGA TTGTTACT | 6585 |
| 301 | ACAAGCUC G CGCAGUUG | 897 | CAACTGCG GGCTAGCTACAACGA GAGCTTGT | 6586 |
| 303 | AAGCUCAC G CAGUUGGG | 216 | CCCAACTG GGCTAGCTACAACGA GTGAGCTT | 6587 |
| 306 | CUCACGCA G UUGGGCAC | 2169 | GTGCCCAA GGCTAGCTACAACGA TGCGTGAG | 6588 |
| 311 | GCAGUUGG G CACUUUUG | 2170 | CAAAAGTG GGCTAGCTACAACGA CCAACTGC | 6589 |
| 313 | AGUUGGGC G CUUUUGAA | 899 | TTCAAAAG GGCTAGCTACAACGA GCCCAACT | 6590 |
| 323 | UUUUGAAG G UCAUUUUC | 2753 | GAAAATGA GGCTAGCTACAACGA CTTCAAAA | 6591 |
| 326 | UGAAGAUC G UUUUCUCA | 901 | TGAGAAAA GGCTAGCTACAACGA GATCTTCA | 6592 |
| 335 | UUUUCUCA G CCUCCAGA | 2171 | TCTGGAGG GGCTAGCTACAACGA TGAGAAAA | 6593 |
| 346 | UCCAGAGG G UGUUCAAU | 2754 | ATTGAACA GGCTAGCTACAACGA CCTCTGGA | 6594 |
| 348 | CAGAGGAU G UUCAAUAA | 2172 | TTATTGAA GGCTAGCTACAACGA ATCCTCTG | 6595 |
| 353 | GAUGUUCA G UAACUGUG | 2755 | CACAGTTA GGCTAGCTACAACGA TGAACATC | 6596 |
| 356 | GUUCAAUA G CUGUGAGG | 2756 | CCTCACAG GGCTAGCTACAACGA TATTGAAC | 6597 |
| 359 | CAAUAACU G UGAGGUGO | 2173 | CCACCTCA GGCTAGCTACAACGA AGTTATTG | 6598 |
| 364 | ACUGUGAG G UGGUCCUU | 2174 | AAGGACCA GGCTAGCTACAACGA CTCACAGT | 6599 |
| 367 | GUGAGGUG G UCCUUGGG | 2175 | CCCAAGGA GGCTAGCTACAACGA CACCTCAC | 6600 |
| 377 | CCUUGGGA G UUUGGAAA | 2757 | TTTCCAAA GGCTAGCTACAACGA TCCCAAGG | 6601 |
| 385 | AUUUGGAA G UUACCUAU | 2758 | ATAGGTAA GGCTAGCTACAACGA TTCCAAAT | 6602 |
| 388 | UGGAAAUU G CCUAUGUG | 44 | CACATAGG GGCTAGCTACAACGA AATTTCCA | 6603 |
| 392 | AAUUACCU G UGUGCAGA | 45 | TCTGCACA GGCTAGCTACAACGA AGGTAATT | 6604 |
| 394 | UUACCUAU G UGCAGAGG | 2176 | CCTCTGCA GGCTAGCTACAACGA ATAGGTAA | 6605 |
| 396 | ACCUAUGU G CAGAGGAA | 2177 | TTCCTCTG GGCTAGCTACAACGA ACATAGGT | 6606 |
| 404 | GCAGAGGA G UUAUGAUC | 2759 | GATCATAA GGCTAGCTACAACGA TCCTCTGC | 6607 |
| 407 | GAGGAAUU G UGAUCUUU | 47 | AAAGATCA GGCTAGCTACAACGA AATTCCTC | 6608 |
| 410 | GAAUUAUG G UCUUUCCU | 2760 | AGGAAAGA GGCTAGCTACAACGA CATAATTC | 6609 |
| 427 | UCUUAAAG G CCAUCCAG | 2761 | CTGGATGG GGCTAGCTACAACGA CTTTAAGA | 6610 |
| 430 | UAAAGACC G UCCAGGAG | 920 | CTCCTGGA GGCTAGCTACAACGA GGTCTTTA | 6611 |
| 439 | UCCAGGAG G UGGCUGGU | 2178 | ACCAGCCA GGCTAGCTACAACGA CTCCTGGA | 6612 |
| 442 | AGGAGGUG G CUGGUUAU | 2179 | ATAACCAG GGCTAGCTACAACGA CACCTCCT | 6613 |
| 446 | GGUGGCUG G UUAUGUCC | 2180 | GGACATAA GGCTAGCTACAACGA CAGCCACC | 6614 |
| 449 | GGCUGGUU G UGUCCUCA | 58 | TGAGGACA GGCTAGCTACAACGA AACCAGCC | 6615 |
| 451 | CUGGUUAU G UCCUCAUU | 2181 | AATGAGGA GGCTAGCTACAACGA ATAACCAG | 6616 |
| 457 | AUGUCCUC G UUGCCCUC | 926 | GAGGGCAA GGCTAGCTACAACGA GAGGACAT | 6617 |
| 460 | UCCUCAUU G CCCUCAAC | 2182 | GTTGAGGG GGCTAGCTACAACGA AATGAGGA | 6618 |
| 467 | UGCCCUCA G CACAGUGG | 2762 | CCACTGTG GGCTAGCTACAACGA TGAGGGCA | 6619 |
| 469 | CCCUCAAC G CAGUGGAG | 931 | CTCCACTG GGCTAGCTACAACGA GTTGAGGG | 6620 |
| 472 | UCAACACA G UGGAGCGA | 2183 | TCGCTCCA GGCTAGCTACAACGA TGTGTTGA | 6621 |
| 477 | ACAGUGGA G CGAAUUCC | 2184 | GGAATTCG GGCTAGCTACAACGA TCCACTGT | 6622 |
| 481 | UGGAGCGA G UUCCUUUG | 2763 | CAAAGGAA GGCTAGCTACAACGA TCGCTCCA | 6623 |
| 494 | UUUGGAAA G CCUGCAGA | 2764 | TCTGCAGG GGCTAGCTACAACGA TTTCCAAA | 6624 |
| 498 | GAAAACCU G CAGAUCAU | 2183 | ATGATCTG GGCTAGCTACAACGA AGGTTTTC | 6625 |
| 502 | ACCUCCAG G UCAUCAGA | 2765 | TCTGATGA GGCTAGCTACAACGA CTGCAGGT | 6626 |
| 505 | UGCAGAUC G UCAGAGGA | 938 | TCCTCTGA GGCTAGCTACAACGA GATCTGCA | 6627 |
| 515 | CAGAGGAA G UAUGUACU | 2766 | AGTACATA GGCTAGCTACAACGA TTCCTCTG | 6628 |
| 517 | GAGGAAAU G UGUACUAC | 69 | GTAGTACA GGCTAGCTACAACGA ATTTCCTC | 6629 |
| 519 | GGAAAUAU G UACUACGA | 2186 | TCGTAGTA GGCTAGCTACAACGA ATATTTCC | 6630 |
| 521 | AAAUAUGU G CUACGAAA | 70 | TTTCGTAG GGCTAGCTACAACGA ACATATTT | 6631 |
| 524 | UAUGUACU G CGAAAAUU | 71 | AATTTTCG GGCTAGCTACAACGA AGTACATA | 6632 |
| 530 | CUACGAAA G UUCCUAUG | 2767 | CATAGGAA GGCTAGCTACAACGA TTTCGTAG | 6633 |
| 536 | AAAUCCCU G UGCCUUAG | 74 | CTAAGGCA GGCTAGCTACAACGA AGGAATTT | 6634 |
| 538 | AUUCCUAU G CCUUAGCA | 2187 | TGCTAAGG GGCTAGCTACAACGA ATAGGAAT | 6635 |
| 544 | AUGCCUUA G CAGUCUUA | 2188 | TAAGACTG GGCTAGCTACAACGA TAAGGCAT | 6636 |
| 547 | CCUUAGCA G UCUUAUCU | 2189 | AGATAAGA GGCTAGCTACAACGA TGCTAAGG | 6637 |
| 552 | GCAGUCUU G UCUAACUA | 79 | TAGTTAGA GGCTAGCTACAACGA AAGACTGC | 6638 |
| 557 | CUUAUCUA G CUAUGAUG | 2768 | CATCATAG GGCTAGCTACAACGA TAGATAAG | 6639 |
| 560 | AUCUAACU G UGAUGCAA | 82 | TTGCATCA GGCTAGCTACAACGA AGTTAGAT | 6640 |
| 563 | UAACUAUG G UGCAAAUA | 2769 | TATTTGCA GGCTAGCTACAACGA CATAGTTA | 6641 |
| 565 | ACUAUGAU G CAAAUAAA | 2190 | TTTATTTG GGCTAGCTACAACGA ATCATAGT | 6642 |
| 569 | UGAUGCAA G UAAAACCG | 2770 | CGGTTTTA GGCTAGCTACAACGA TTGCATCA | 6643 |
| 574 | CAAAUAAA G CCGGACUG | 2771 | CAGTCCGG GGCTAGCTACAACGA TTTATTTG | 6644 |
| 579 | AAAACCGG G CUGAAGGA | 2772 | TCCTTCAG GGCTAGCTACAACGA CCGGTTTT | 6645 |
| 588 | CUGAAGGA G CUGCCCAU | 2191 | ATGGGCAG GGCTAGCTACAACGA TCCTTCAG | 6646 |
| 591 | AAGGAGCU G CCCAUGAG | 2192 | CTCATGGG GGCTAGCTACAACGA AGCTCCTT | 6647 |
| 595 | AGCUGCCC G UGAGAAAU | 955 | ATTTCTCA GGCTAGCTACAACGA GGGCAGCT | 6648 |
| 602 | CAUGAGAA G UUUACAGG | 2773 | CCTGTAAA GGCTAGCTACAACGA TTCTCATG | 6649 |
| 606 | AGAAAUUU G CAGGAAAU | 86 | ATTTCCTG GGCTAGCTACAACGA AAATTTCT | 6650 |
| 613 | UACAGGAA G UCCUGCAU | 2774 | ATGCAGGA GGCTAGCTACAACGA TTCCTGTA | 6651 |
| 618 | GAAAUCCU G CAUGGCGC | 2193 | GCGCCATG GGCTAGCTACAACGA AGGATTTC | 6652 |
| 620 | AAUCCUGC G UGGCGCCG | 959 | CGGCGCCA GGCTAGCTACAACGA GCAGGATT | 6653 |
| 623 | CCUGCAUG G CGCCGUGC | 2194 | GCACGGCG GGCTAGCTACAACGA CATGCAGG | 6654 |
| 625 | UGCAUGGC G CCGUGCGG | 2195 | CCGCACGG GGCTAGCTACAACGA GCCATGCA | 6655 |
| 628 | AUGGCGCC G UGCGGUUC | 2196 | GAACCGCA GGCTAGCTACAACGA GGCGCCAT | 6656 |
| 630 | GGCGCCGU G CGCUUCAG | 2197 | CTGAACCG GGCTAGCTACAACGA ACGGCGCC | 6657 |
| 633 | GCCGUGCG G UUCAGCAA | 2198 | TTGCTGAA GGCTAGCTACAACGA CGCACGGC | 6658 |
| 638 | GCGGUUCA G CAACAACC | 2199 | GGTTGTTG GCCTAGCTACAACGA TGAACCGC | 6659 |
| 641 | GUUCAGCA G CAACCCUG | 2775 | CAGGGTTG GGCTAGCTACAACGA TGCTGAAC | 6660 |
| 644 | CAGCAACA G CCCUGCCC | 2776 | GGGCAGGG GGCTAGCTACAACGA TGTTGCTG | 6661 |
| 649 | ACAACCCU G CCCUGUGC | 2200 | GCACAGGG GGCTAGCTACAACGA AGGGTTGT | 6662 |
| 654 | CCUGCCCU G UGCAACGU | 2201 | ACGTTGCA GGCTAGCTACAACGA AGGGCAGG | 6663 |
| 656 | UGCCCUGU G CAACGUGG | 2202 | CCACGTTG GGCTAGCTACAACGA ACAGGGCA | 6664 |
| 659 | CCUGUGCA G CGUGGAGA | 2777 | TCTCCACG GGCTAGCTACAACGA TGCACAGG | 6665 |
| 661 | UGUGCAAC G UGGAGAGC | 2203 | GCTCTCCA GGCTAGCTACAACGA GTTGCACA | 6666 |
| 668 | CGUGGAGA G CAUCCAGU | 2204 | ACTGGATG GGCTAGCTACAACGA TCTCCACG | 6667 |
| 670 | UGGAGAGC G UCCAGUGG | 971 | CCACTGGA GGCTAGCTACAACGA GCTCTCCA | 6668 |
| 675 | AGCAUCCA G UGGCGGGA | 2205 | TCCCGCCAGGCTAGCTACAACGA TGGATGCT6669 | |
| 678 | AUCCAGUG G CGGGACAU | 2206 | ATGTCCCGGGCTAGCTACAACGA CACTGGAT6670 | |
| 683 | GUGGCGGG G CAUAGUCA | 2778 | TGACTATGGGCTAGCTACAACGA CCCGCCAC6671 | |
| 685 | GGCGGGAC G UAGUCAGC | 974 | GCTGACTAGGCTAGCTACAACGA GTCCCGCC6672 | |
| 688 | GGGACAUA G UCAGCAGU | 2207 | ACTGCTGAGGCTAGCTACAACGA TATGTCCC6673 | |
| 692 | CAUAGUCA G CAGUGACU | 2208 | AGTCACTG GGCTAGCTACAACGA TGACTATG | 6674 |
| 695 | AGUCAGCA G UGACUUUC | 2209 | GAAAGTCA GGCTAGCTACAACGA TGCTGACT | 6675 |
| 698 | CAGCAGUG G CUUUCUCA | 2779 | TGAGAAAG GGCTAGCTACAACGA CACTGCTG | 6676 |
| 707 | CUUUCUCA G CAACAUGU | 2210 | ACATGTTG GGCTAGCTACAACGA TGAGAAAG | 6677 |
| 710 | UCUCAGCA G CAUGUCGA | 2780 | TCGACATG GGCTAGCTACAACGA TGCTGAGA | 6678 |
| 712 | UCAGCAAC G UGUCGAUG | 981 | CATCGACA GGCTAGCTACAACGA GTTGCTGA | 6679 |
| 714 | AGCAACAU G UCGAUGGA | 2211 | TCCATCGA GGCTAGCTACAACGA ATGTTGCT | 6680 |
| 718 | ACAUGUCG G UGGACUUC | 2781 | GAAGTCCA GGCTAGCTACAACGA CGACATGT | 6681 |
| 722 | GUCGAUGG G CUUCCAGA | 2782 | TCTGGAAG GGCTAGCTACAACGA CCATCGAC | 6682 |
| 731 | CUUCCAGA G CCACCUGG | 2783 | CCAGGTGG GGCTAGCTACAACGA TCTGGAAG | 6683 |
| 734 | CCAGAACC G CCUGGGCA | 986 | TGCCCAGG GGCTAGCTACAACGA GGTTCTGG | 6684 |
| 740 | CCACCUGG G CAGCUGCC | 2212 | GGCAGCTG GGCTAGCTACAACGA CCAGGTGG | 6685 |
| 743 | CCUGGGCA G CUGCCAAA | 2213 | TTTGGCAG GGCTAGCTACAACGA TGCCCAGG | 6686 |
| 746 | GGGCAGCU G CCAAAAGU | 2214 | ACTTTTGG GGCTAGCTACAACGA AGCTGCCC | 6687 |
| 753 | UGCCAAAA G UGUGAUCC | 2215 | GGATCACA GGCTAGCTACAACGA TTTTGGCA | 6688 |
| 755 | CCAAAAGU G UGAUCCAA | 2216 | TTGGATCA GGCTAGCTACAACGA ACTTTTGG | 6689 |
| 758 | AAAGUGUG G UCCAAGCU | 2784 | AGCTTGGA GGCTAGCTACAACGA CACACTTT | 6690 |
| 764 | UGAUCCAA G CUGUCCCA | 2217 | TGGGACAG GGCTAGCTACAACGA TTGGATCA | 6691 |
| 767 | UCCAAGCU G UCCCAAUG | 2218 | CATTGGGA GGCTAGCTACAACGA AGCTTGGA | 6692 |
| 773 | CUGUCCCA G UGGGAGCU | 2785 | AGCTCCCA GGCTAGCTACAACGA TGGGACAG | 6693 |
| 779 | CAAUGGGA G CUGCUGGG | 2219 | CCCAGCAG GGCTAGCTACAACGA TCCCATTG | 6694 |
| 782 | UGGGAGCU G CUGGGGUG | 2220 | CACCCCAG GGCTAGCTACAACGA AGCTCCCA | 6695 |
| 788 | CUGCUGGG G UGCAGGAG | 2221 | CTCCTGCA GGCTAGCTACAACGA CCCAGCAG | 6696 |
| 790 | GCUGGGGU G CAGGAGAG | 2222 | CTCTCCTG GGCTAGCTACAACGA ACCCCAGC | 6697 |
| 803 | AGAGGAGA G CUGCCAGA | 2786 | TCTGGCAG GGCTAGCTACAACGA TCTCCTCT | 6698 |
| 806 | GGAGAACU G CCAGAAAC | 2223 | GTTTCTGG GGCTAGCTACAACGA AGTTCTCC | 6699 |
| 813 | UGCCAGAA G CUGACCAA | 2787 | TTGGTCAG GGCTAGCTACAACGA TTCTGGCA | 6700 |
| 817 | AGAAACUG G CCAAAAUC | 2788 | GATTTTGG GGCTAGCTACAACGA CAGTTTCT | 6701 |
| 823 | UGACCAAA G UCAUCUGU | 2789 | ACAGATGA GGCTAGCTACAACGA TTTGGTCA | 6702 |
| 826 | CCAAAAUC G UCUGUGCC | 1008 | GGCACAGA GGCTAGCTACAACGA GATTTTGG | 6703 |
| 830 | AAUCAUCU G UGCCCAGC | 2224 | GCTGGGCA GGCTAGCTACAACGA AGATGATT | 6704 |
| 832 | UCAUCUGU G CCCAGCAG | 2225 | CTGCTGGG GGCTAGCTACAACGA ACAGATGA | 6705 |
| 837 | UGUGCCCA G CAGUGCUC | 2226 | GAGCACTG GGCTAGCTACAACGA TGGGCACA | 6706 |
| 840 | GCCCAGCA G UGCUCCGG | 2227 | CCGGAGCA GGCTAGCTACAACGA TGCTGGGC | 6707 |
| 842 | CCAGCAGU G CUCCGGGC | 2228 | GCCCGGAG GGCTAGCTACAACGA ACTGCTGG | 6708 |
| 849 | UGCUCCGG G CGCUGCCG | 2229 | CGGCAGCG GGCTAGCTACAACGA CCGGAGCA | 6709 |
| 851 | CUCCGGGC G CUGCCGUG | 2230 | CACGGCAG GGCTAGCTACAACGA GCCCGGAG | 6710 |
| 854 | CGGGCGCU G CCGUGGCA | 2231 | TGCCACGG GGCTAGCTACAACGA AGCGCCCG | 6711 |
| 857 | GCGCUGCC G UGGCAAGU | 2232 | ACTTGCCA GGCTAGCTACAACGA GGCAGCGC | 6712 |
| 860 | CUGCCGUG G CAAGUCCC | 2233 | GGGACTTG GGCTAGCTACAACGA CACGGCAG | 6713 |
| 864 | CGUGGCAA G UCCCCCAG | 2234 | CTGGGGGA GGCTAGCTACAACGA TTGCCACG | 6714 |
| 872 | GUCCCCCA G UGACUGCU | 2235 | AGCAGTCA GGCTAGCTACAACGA TGGGGGAC | 6715 |
| 875 | CCCCAGUG G CUGCUGCC | 2790 | GGCAGCAG GGCTAGCTACAACGA CACTGGGG | 6716 |
| 878 | CAGUGACU G CUGCCACA | 2236 | TGTGGCAG GGCTAGCTACAACGA AGTCACTG | 6717 |
| 881 | UGACUGCU G CCACAACC | 2237 | GGTTGTGG GGCTAGCTACAACGA AGCAGTCA | 6718 |
| 884 | CUGCUGCC G CAACCAGU | 1027 | ACTGGTTG GGCTAGCTACAACGA GGCAGCAG | 6719 |
| 887 | CUGCCACA G CCAGUGUG | 2791 | CACACTGG GGCTAGCTACAACGA TGTGGCAG | 6720 |
| 891 | CACAACCA G UCUCCUGC | 2238 | GCAGCACA GGCTAGCTACAACGA TGGTTGTG | 6721 |
| 893 | CAACCAGU G UGCUGCAG | 2239 | CTGCAGCA GGCTAGCTACAACGA ACTGGTTG | 6722 |
| 895 | ACCAGUGU G CUGCAGGC | 2240 | GCCTGCAG GGCTAGCTACAACGA ACACTGGT | 6723 |
| 898 | AGUGUGCU G CAGGCUGC | 2241 | GCAGCCTG GGCTAGCTACAACGA AGCACACT | 6724 |
| 902 | UGCUGCAG G CUGCACAG | 2242 | CTGTGCAG GGCTAGCTACAACGA CTGCAGCA | 6725 |
| 905 | UGCAGGCU G CACAGGCC | 2243 | GGCCTGTG GGCTAGCTACAACGA AGCCTGCA | 6726 |
| 907 | CAGGCUGC G CAGGCCCC | 1034 | GGGCCCTG GGCTAGCTACAACGA GCAGCCTG | 6727 |
| 911 | CUGCACAG G CCCCCGGG | 2244 | CCCGGGGG GGCTAGCTACAACGA CTGTGCAG | 6728 |
| 923 | CCGGGAGA G CGACUGCC | 2245 | GGCAGTCG GGCTAGCTACAACGA TCTCCCGG | 6729 |
| 926 | GGAGAGCG G CUGCCUGG | 2792 | CCAGGCAG GGCTAGCTACAACGA CGCTCTCC | 6730 |
| 929 | GAGCGACU G CCUGGUCU | 2246 | AGACCAGG GGCTAGCTACAACGA AGTCGCTC | 6731 |
| 934 | ACUGCCUG G UCUGCCGC | 2247 | GCGGCAGA GGCTAGCTACAACGA CAGGCAGT | 6732 |
| 938 | CCUGGUCU G CCGCAAAU | 2248 | ATTTGCGG GGCTAGCTACAACGA AGACCAGG | 6733 |
| 941 | GGUCUGCC G CAAAUUCC | 2249 | GGAATTTG GGCTAGCTACAACGA GGCAGACC | 6734 |
| 945 | UGCCGCAA G UUCCGAGA | 2793 | TCTCGGAA GGCTAGCTACAACGA TTGCGGCA | 6735 |
| 953 | AUUCCGAG G CGAAGCCA | 2794 | TGGCTTCG GGCTAGCTACAACGA CTCGGAAT | 6736 |
| 958 | GAGACGAA G CCACGUGC | 2250 | GCACGTGG GGCTAGCTACAACGA TTCGTCTC | 6737 |
| 961 | ACGAAGCC G CGUGCAAG | 1048 | CTTGCACG GGCTAGCTACAACGA GGCTTCGT | 6738 |
| 963 | GAAGCCAC G UGCAAGGA | 2251 | TCCTTGCA GGCTAGCTACAACGA GTGGCTTC | 6739 |
| 965 | AGCCACGU G CAAGGACA | 2252 | TGTCCTTG GGCTAGCTACAACGA ACGTGGCT | 6740 |
| 971 | GUGCAAGG G CACCUGCC | 2795 | GGCAGGTG GGCTAGCTACAACGA CCTTGCAC | 6741 |
| 973 | GCAAGGAC G CCUGCCCC | 1050 | GGGGCAGG GGCTAGCTACAACGA GTCCTTGC | 6742 |
| 977 | GGACACCU G CCCCCCAC | 2253 | GTGGGGGG GGCTAGCTACAACGA AGGTGTCC | 6743 |
| 984 | UGCCCCCC G CUCAUGCU | 1058 | AGCATGAG GGCTAGCTACAACGA GGGGGGCA | 6744 |
| 988 | CCCCACUC G UGCUCUAC | 1060 | GTAGAGCA GGCTAGCTACAACGA GAGTGGGG | 6745 |
| 990 | CCACUCAU G CUCUACAA | 2254 | TTGTAGAG GGCTAGCTACAACGA ATGAGTGG | 6746 |
| 995 | CAUGCUCU G CAACCCCA | 111 | TGGGGTTG GGCTAGCTACAACGA AGAGCATG | 6747 |
| 998 | GCUCUACA G CCCCACCA | 2796 | TGGTGGGG GGCTAGCTACAACGA TGTAGAGC | 6748 |
| 1003 | ACAACCCC G CCACGUAC | 1067 | GTACGTGG GGCTAGCTACAACGA GGGGTTGT | 6749 |
| 1006 | ACCCCACC G CGUACCAG | 1069 | CTGGTACG GGCTAGCTACAACGA GGTGGGGT | 6750 |
| 1008 | CCCACCAC G UACCAGAU | 2255 | ATCTGGTA GGCTAGCTACAACGA GTGGTGGG | 6751 |
| 1010 | CACCACGU G CCAGAUGG | 112 | CCATCTGG GGCTAGCTACAACGA ACGTGGTG | 6752 |
| 1015 | CGUACCAG G UGGAUGUG | 2797 | CACATCCA GGCTAGCTACAACGA CTGGTACG | 6753 |
| 1019 | CCAGAUGG G UGUGAACC | 2798 | GGTTCACA GGCTAGCTACAACGA CCATCTGG | 6754 |
| 1021 | AGAUGGAU G UGAACCCC | 2256 | GGGGTTCA GCCTACCTACAACGA ATCCATCT | 6755 |
| 1025 | GGAUGUGA G CCCCGAGG | 2799 | CCTCGGGG GGCTAGCTACAACGA TCACATCC | 6756 |
| 1034 | CCCCGAGG G CAAAUACA | 2257 | TGTATTTG GGCTAGCTACAACGA CCTCGGGG | 6757 |
| 1038 | GAGGGCAA G UACAGCUU | 2800 | AAGCTGTA GGCTAGCTACAACGA TTGCCCTC | 6758 |
| 1040 | GGGCAAAU G CAGCUUUG | 113 | CAAAGCTG GGCTAGCTACAACGA ATTTGCCC | 6759 |
| 1043 | CAAAUACA G CUUUGGUG | 2258 | CACCAAAG GGCTAGCTACAACGA TGTATTTG | 6760 |
| 1049 | CAGCUUUG G UGCCACCU | 2259 | AGGTGGCA GGCTAGCTACAACGA CAAAGCTG | 6761 |
| 1051 | GCUUUGGU G CCACCUGC | 2260 | GCAGCTGG GCCTAGCTACAACGA ACCAAAGC | 6762 |
| 1054 | UUGGUGCC G CCUGCGUG | 1079 | CACGCAGG GGCTAGCTACAACGA GGCACCAA | 6763 |
| 1058 | UGCCACCU G CGUCAAGA | 2261 | TCTTCACG GGCTAGCTACAACGA AGGTGGCA | 6764 |
| 1060 | CCACCUGC G UGAAGAAC | 2262 | CTTCTTCA GGCTAGCTACAACGA GCAGGTGG | 6765 |
| 1068 | GUGAAGAA G UGUCCCCG | 2263 | CGGGGACA GGCTAGCTACAACGA TTCTTCAC | 6766 |
| 1070 | GAAGAAGU G UCCCCGUA | 2264 | TACGGGGA GGCTAGCTACAACGA ACTTCTTC | 6767 |
| 1076 | GUGUCCCC G UAAUUAUG | 2265 | CATAATTA GGCTAGCTACAACGA GGGCACAC | 6768 |
| 1079 | UCCCCGUA G UUAUGUGG | 2801 | CCACATAA GGCTAGCTACAACGA TACGGGGA | 6769 |
| 1082 | CCGUAAUU G UGUGGUGA | 119 | TCACCACA GGCTAGCTACAACGA AATTACGG | 6770 |
| 1084 | GUAAUUAU G UGGUGACA | 2266 | TGTCACCA GGCTAGCTACAACGA ATAATTAC | 6771 |
| 1087 | AUUAUGUG G UGACAGAU | 2267 | ATCTGTCA GGCTAGCTACAACGA CACATAAT | 6772 |
| 1090 | AUGUGGUG G CAGAUCAC | 2802 | GTGATCTG GGCTAGCTACAACGA CACCACAT | 6773 |
| 1094 | GGUGACAG G UCACGGCU | 2803 | AGCCGTGA GGCTAGCTACAACGA CTGTCACC | 6774 |
| 1097 | GACAGAUC G CGGCUCGU | 1086 | ACGAGCCG GGCTAGCTACAACGA GATCTGTC | 6775 |
| 1100 | AGAUCACG G CUCGUGCG | 2268 | CGCACGAG GGCTAGCTACAACGA CGTGATCT | 6776 |
| 1104 | CACGGCUC G UGCGUCCG | 2269 | CGGACGCA GGCTAGCTACAACGA GAGCCGTG | 6777 |
| 1106 | CGGCUCGU G CGUCCGAG | 2270 | CTCGGACG GGCTAGCTACAACGA ACGAGCCG | 6778 |
| 1108 | GCUCGUGC G UCCGAGCC | 2271 | GGCTCGGA GGCTAGCTACAACGA GCACGAGC | 6779 |
| 1114 | GCGUCCGA G CCUGUGGG | 2272 | CCCACAGG GGCTAGCTACAACGA TCGGACGC | 6780 |
| 1118 | CCGAGCCU G UGGGGCCG | 2273 | CGGCCCCA GGCTAGCTACAACGA AGGCTCGG | 6781 |
| 1123 | CCUGUGGG G CCGACAGC | 2274 | GCTGTCGG GGCTAGCTACAACGA CCCACAGG | 6782 |
| 1127 | UGGGGCCG G CAGCUAUG | 2804 | CATAGCTG GGCTAGCTACAACGA CGGCCCCA | 6783 |
| 1130 | GGCCGACA G CUAUGAGA | 2275 | TCTCATAG GGCTAGCTACAACGA TGTCGGCC | 6784 |
| 1133 | CGACAGCU G UGAGAUGG | 123 | CCATCTCA GGCTAGCTACAACGA AGCTGTCG | 6785 |
| 1138 | GCUAUGAG G UGGAGGAA | 2805 | TTCCTCCA GGCTAGCTACAACGA CTCATAGC | 6786 |
| 1148 | GGAGGAAG G CGGCGUCC | 2806 | GGACGCCG GGCTAGCTACAACGA CTTCCTCC | 6787 |
| 1151 | GGAAGACG G CGUCCGCA | 2276 | TGCGGACG GGCTAGCTACAACGA CGTCTTCC | 6788 |
| 1153 | AAGACGGC G UCCGCAAG | 2277 | CTTGCGGA GGCTAGCTACAACGA GCCGTCTT | 6789 |
| 1157 | CGGCGUCC G CAAGUGUA | 2278 | TACACTTG GGCTAGCTACAACGA GGACGCCG | 6790 |
| 1161 | GUCCGCAA G UGUAAGAA | 2279 | TTCTTACA GGCTAGCTACAACGA TTGCGGAC | 6791 |
| 1163 | CCGCAAGU G UAAGAAGU | 2280 | ACTTCTTA GGCTAGCTACAACGA ACTTGCGG | 6792 |
| 1170 | UGUAAGAA G UGCGAAGG | 2281 | CCTTCGCA GGCTAGCTACAACGA TTCTTACA | 6793 |
| 1172 | UAAGAAGU G CGAAGGGC | 2282 | GCCCTTCG GGCTAGCTACAACGA ACTTCTTA | 6794 |
| 1179 | UGCGAAGG G CCUUGCCG | 2283 | CGGCAAGG GGCTAGCTACAACGA CCTTCGCA | 6795 |
| 1184 | AGGGCCUU G CCGCAAAG | 2284 | CTTTGCGG GGCTAGCTACAACGA AAGGCCCT | 6796 |
| 1187 | GCCUUGCC G CAAAGUGU | 2285 | ACACTTTG GGCTAGCTACAACGA GGCAAGGC | 6797 |
| 1192 | GCCGCAAA G UGUGUAAC | 2286 | GTTACACA GGCTAGCTACAACGA TTTGCGGC | 6798 |
| 1194 | CGCAAAGU G UGUAACGG | 2287 | CCGTTACA GGCTAGCTACAACGA ACTTTGCG | 6799 |
| 1196 | CAAAGUGU G UAACGGAA | 2288 | TTCCGTTA GGCTAGCTACAACGA ACACTTTG | 6800 |
| 1199 | AGUGUGUA G CGGAAUAG | 2807 | CTATTCCG GGCTAGCTACAACGA TACACACT | 6801 |
| 1204 | GUAACGGA G UAGGUAUU | 2808 | AATACCTA GGCTAGCTACAACGA TCCGTTAC | 6802 |
| 1208 | CGGAAUAG G UAUUGGUG | 2289 | CACCAATA GGCTAGCTACAACGA CTATTCCG | 6803 |
| 1210 | GAAUAGGU G UUGGUGAA | 129 | TTCACCAA GGCTAGCTACAACGA ACCTATTC | 6804 |
| 1214 | AGGUAUUG G UGAAUUUA | 2290 | TAAATTCA GGCTAGCTACAACGA CAATACCT | 6805 |
| 1218 | AUUGGUGA G UUUAAAGA | 2809 | TCTTTAAA GGCTAGCTACAACGA TCACCAAT | 6806 |
| 1226 | AUUUAAAG G CUCACUCU | 2810 | AGAGTGAG GGCTAGCTACAACGA CTTTAAAT | 6807 |
| 1230 | AAAGACUC G CUCUCCAU | 1101 | ATGGAGAG GGCTAGCTACAACGA GAGTCTTT | 6808 |
| 1237 | CACUCUCC G UAAAUGCU | 1105 | AGCATTTA GGCTAGCTACAACGA GGAGAGTG | 6809 |
| 1241 | CUCCAUAA G UGCUACGA | 2811 | TCGTAGCA GGCTAGCTACAACGA TTATGGAG | 6810 |
| 1243 | CCAUAAAU G CUACGAAU | 2291 | ATTCGTAG GGCTAGCTACAACGA ATTTATGG | 6811 |
| 1246 | UAAAUGCU G CGAAUAUU | 138 | AATATTCG GGCTAGCTACAACGA AGCATTTA | 6812 |
| 1250 | UGCUACGA G UAUUAAAC | 2812 | GTTTAATA GGCTAGCTACAACGA TCGTAGCA | 6813 |
| 1252 | CUACGAAU G UUAAACAC | 139 | GTGTTTAA GGCTAGCTACAACGA ATTCGTAG | 6814 |
| 1257 | AAUAUUAA G CACUUCAA | 2813 | TTGAAGTG GGCTAGCTACAACGA TTAATATT | 6815 |
| 1259 | UAUUAAAC G CUUCAAAA | 1107 | TTTTGAAG GGCTAGCTACAACGA GTTTAATA | 6816 |
| 1268 | CUUCAAAA G CUGCACCU | 2814 | AGGTGCAG GGCTAGCTACAACGA TTTTGAAG | 6817 |
| 1271 | CAAAAACU G CACCUCCA | 2292 | TGGAGGTG GGCTAGCTACAACGA AGTTTTTG | 6818 |
| 1273 | AAAACUGC G CCUCCAUC | 1111 | GATGGAGG GGCTAGCTACAACGA GCAGTTTT | 6819 |
| 1279 | GCACCUCC G UCAGUGGC | 1115 | GCCACTGA GGCTAGCTACAACGA GGAGGTGC | 6820 |
| 1283 | CUCCAUCA G UGGCGAUC | 2293 | GATCGCCA GGCTAGCTACAACGA TGATGGAG | 6821 |
| 1286 | CAUCAGUG G CGAUCUCC | 2294 | GGAGATCG GGCTAGCTACAACGA CACTGATG | 6822 |
| 1289 | CAGUGGCG G UCUCCACA | 2815 | TGTGGAGA GGCTAGCTACAACGA CGCCACTG | 6823 |
| 1295 | CGAUCUCC G CAUCCUGC | 1119 | GCAGGATG GGCTAGCTACAACGA GGAGATCG | 6824 |
| 1297 | AUCUCCAC G UCCUGCCG | 1120 | CGGCAGGA GGCTAGCTACAACGA GTGGAGAT | 6825 |
| 1302 | CACAUCCU G CCGGUGGC | 2295 | GCCACCGG GGCTAGCTACAACGA AGGATGTG | 6826 |
| 1306 | UCCUGCCG G UGGCAUUU | 2296 | AAATGCCA GGCTAGCTACAACGA CGGCAGGA | 6827 |
| 1309 | UGCCGGUG G CAUUUAGG | 2297 | CCTAAATG GGCTAGCTACAACGA CACCGGCA | 6828 |
| 1311 | CCGGUGGC G UUUAGGGG | 1124 | CCCCTAAA GGCTAGCTACAACGA GCCACCGG | 6829 |
| 1319 | AUUUAGGG G UGACUCCU | 2298 | AGGAGICA GGCTAGCTACAACGA CCCTAAAT | 6830 |
| 1322 | UAGGGGUG G CUCCUUCA | 2816 | TGAAGGAG GGCTAGCTACAACGA CACCCCTA | 6831 |
| 1330 | ACUCCUUC G CACAUACU | 1128 | AGTATGTG GGCTAGCTACAACGA GAAGGAGT | 6832 |
| 1332 | UCCUUCAC G CAUACUCC | 1129 | GGAGTATG GGCTAGCTACAACGA GTGAAGGA | 6833 |
| 1334 | CUUCACAC G UACUCCUC | 1130 | GAGGAGTA GGCTAGCTACAACGA GTGTGAAG | 6834 |
| 1336 | UCACACAU G CUCCUCCU | 155 | AGGAGGAG GGCTAGCTACAACGA ATGTGTGA | 6835 |
| 1349 | UCCUCUGG G UCCACAGG | 2817 | CCTGTGGA GGCTAGCTACAACGA CCAGAGGA | 6836 |
| 1353 | CUGGAUCC G CAGGAACU | 1138 | AGTTCCTG GGCTAGCTACAACGA GGATCCAG | 6837 |
| 1359 | CCACAGGA G CUGGAUAU | 2818 | ATATCCAG GGCTAGCTACAACGA TCCTGTGG | 6838 |
| 1364 | GGAACUGG G UAUUCUGA | 2819 | TCAGAATA GGCTAGCTACAACGA CCAGTTCC | 6839 |
| 1366 | AACUGGAU G UUCUGAAA | 160 | TTTCAGAA GGCTAGCTACAACGA ATCCAGTT | 6840 |
| 1375 | UUCUGAAA G CCGUAAAG | 2820 | CTTTACGG GGCTAGCTACAACGA TTTCAGAA | 6841 |
| 1378 | UGAAAACC G UAAAGGAA | 2299 | TTCCTTTA GGCTAGCTACAACGA GGTTTTCA | 6842 |
| 1387 | UAAAGGAA G UCACAGGG | 2821 | CCCTGTGA GGCTAGCTACAACGA TTCCTTTA | 6843 |
| 1390 | AGGAAAUC G CAGGGUUU | 1143 | AAACCCTG GGCTAGCTACAACGA GATTTCCT | 6844 |
| 1395 | AUCACAGG G UUUUUGCU | 2300 | AGCAAAAA GGCTAGCTACAACGA CCTGTGAT | 6845 |
| 1401 | GGGUUUUU G CUGAUUCA | 2301 | TGAATCAG GGCTAGCTACAACGA AAAAACCC | 6846 |
| 1405 | UUUUGCUG G UUCAGGCU | 2822 | AGCCTGAA GGCTAGCTACAACGA CAGCAAAA | 6847 |
| 1411 | UGAUUCAG G CUUGGCCU | 2302 | AGGCCAAG GGCTAGCTACAACGA CTGAATCA | 6848 |
| 1416 | CAGGCUUG G CCUGAAAA | 2303 | TTTTCAGG GGCTAGCTACAACGA CAAGCCTG | 6849 |
| 1424 | GCCUGAAA G CAGGACGG | 2823 | CCGTCCTG GGCTAGCTACAACGA TTTCAGGC | 6850 |
| 1429 | AAAACAGG G CGGACCUC | 2824 | GAGGTCCG GGCTAGCTACAACGA CCTGTTTT | 6851 |
| 1433 | CAGGACGG G CCUCCAUG | 2825 | CATGGAGG GGCTAGCTACAACGA CCGTCCTG | 6852 |
| 1439 | GGACCUCC G UGCCUUUG | 1154 | CAAAGGCA GGCTAGCTACAACGA GGAGGTCC | 6853 |
| 1441 | ACCUCCAU G CCUUUGAG | 2304 | CTCAAAGG GGCTAGCTACAACGA ATGGAGGT | 6854 |
| 1451 | CUUUGAGA G CCUAGAAA | 2826 | TTTCTAGG GGCTAGCTACAACGA TCTCAAAG | 6855 |
| 1459 | ACCUAGAA G UCAUACGC | 2827 | GCGTATGA GGCTAGCTACAACGA TTCTAGGT | 6856 |
| 1462 | UAGAAAUC G UACGCGGC | 1159 | GCCGCGTA GGCTAGCTACAACGA GATTTCTA | 6857 |
| 1464 | GAAAUCAU G CGCGGCAG | 177 | CTGCCGCG GGCTAGCTACAACGA ATGATTTC | 6858 |
| 1466 | AAUCAUAC G CGGCAGGA | 2305 | TCCTGCCG GGCTAGCTACAACGA GTATGATT | 6859 |
| 1469 | CAUACGCG G CAGGACCA | 2306 | TGGTCCTG GGCTAGCTACAACGA CGCGTATG | 6860 |
| 1474 | GCGGCAGG G CCAAGCAA | 2828 | TTGCTTGG GGCTAGCTACAACGA CCTGCCGC | 6861 |
| 1479 | AGGACCAA G CAACAUGG | 2307 | CCATGTTG GGCTAGCTACAACGA TTGGTCCT | 6862 |
| 1482 | ACCAAGCA G CAUGGUCA | 2829 | TGACCATG GGCTAGCTACAACGA TGCTTGGT | 6863 |
| 1484 | CAAGCAAC G UGGUCAGU | 1164 | ACTGACCA GGCTAGCTACAACGA GTTGCTTG | 6864 |
| 1487 | GCAACAUG G UCAGUUUU | 2308 | AAAACTGA GGCTAGCTACAACGA CATGTTGC | 6865 |
| 1491 | CAUGGUCA G UUUUCUCU | 2309 | AGAGAAAA GGCTAGCTACAACGA TGACCATG | 6866 |
| 1501 | UUUCUCUU G CAGUCGUC | 2310 | GACGACTG GGCTAGCTACAACGA AAGAGAAA | 6867 |
| 1504 | CUCUUGCA G UCGUCAGC | 2311 | GCTGACGA GGCTAGCTACAACGA TGCAAGAG | 6868 |
| 1507 | UUGCAGUC G UCAGCCUG | 2312 | CAGGCTGA GGCTAGCTACAACGA GACTGCAA | 6869 |
| 1511 | AGUCGUCA G CCUGAACA | 2313 | TGTTCAGG GGCTAGCTACAACGA TGACGACT | 6870 |
| 1517 | CAGCCUGA G CAUAACAU | 2830 | ATGTTATG GGCTAGCTACAACGA TCAGGCTG | 6871 |
| 1519 | GCCUGAAC G UAACAUCC | 1172 | GGATGTTA GGCTAGCTACAACGA GTTCAGGC | 6872 |
| 1522 | UGAACAUA G CAUCCUUG | 2831 | CAAGGATG GGCTAGCTACAACGA TATGTTCA | 6873 |
| 1524 | AACAUAAC G UCCUUGGG | 1173 | CCCAAGGA GGCTAGCTACAACGA GTTATGTT | 6874 |
| 1533 | UCCUUGGG G UUACGCUC | 2832 | GAGCGTAA GGCTAGCTACAACGA CCCAAGGA | 6875 |
| 1536 | UUGGGAUU G CGCUCCCU | 191 | AGGGAGCG GGCTAGCTACAACGA AATCCCAA | 6876 |
| 1538 | GGGAUUAC G CUCCCUCA | 2314 | TGAGGGAG GGCTAGCTACAACGA GTAATCCC | 6877 |
| 1552 | UCAAGGAG G UAAGUGAU | 2833 | ATCACTTA GGCTAGCTACAACGA CTCCTTGA | 6878 |
| 1556 | GGAGAUAA G UGAUGGAG | 2315 | CTCCATCA GGCTAGCTACAACGA TTATCTCC | 6879 |
| 1559 | GAUAAGUG G UGGAGAUG | 2834 | CATCTCCA GGCTAGCTACAACGA CACTTATC | 6880 |
| 1565 | UGAUGGAG G UGUGAUAA | 2835 | TTATCACA GGCTAGCTACAACGA CTCCATCA | 6881 |
| 1567 | AUGGAGAU G UGAUAAUU | 2316 | AATTATCA GGCTAGCTACAACGA ATCTCCAT | 6882 |
| 1570 | GAGAUGUG G UAAUUUCA | 836 | TGAAATTA GGCTAGCTACAACGA CACATCTC | 6883 |
| 1573 | AUGUGAUA G UUUCAGGA | 2837 | TCCTGAAA GGCTAGCTACAACGA TATCACAT | 6884 |
| 1583 | UUCAGGAA G CAAAAAUU | 2838 | AATTTTTG GGCTAGCTACAACGA TTCCTGAA | 6885 |
| 1589 | AAACAAAA G UUUGUGCU | 2839 | AGCACAAA GGCTAGCTACAACGA TTTTGTTT | 6886 |
| 1593 | AAAAAUUU G UGCUAUGC | 2317 | GCATAGCA GGCTAGCTACAACGA AAATTTTT | 6887 |
| 1595 | AAAUUUGU G CUAUGCAA | 2318 | TTGCATAG GGCTAGCTACAACGA ACAAATTT | 6888 |
| 1598 | UUUGUGCU G UGCAAAUA | 201 | TATTTGCA GGCTAGCTACAACGA AGCACAAA | 6889 |
| 1600 | UGUGCUAU G CAAAUACA | 2319 | TGTATTTG GGCTAGCTACAACGA ATAGCACA | 6890 |
| 1604 | CUAUGCAA G UACAAUAA | 2840 | TTATTGTA GGCTAGCTACAACGA TTGCATAG | 6891 |
| 1606 | AUGCAAAU G CAAUAAAC | 202 | GTTTATTG GGCTAGCTACAACGA ATTTGCAT | 6892 |
| 1609 | CAAAUACA G UAAACUGG | 2841 | CCAGTTTA GGCTAGCTACAACGA TGTATTTG | 6893 |
| 1613 | UACAAUAA G CUGGAAAA | 2842 | TTTTCCAG GGCTAGCTACAACGA TTATTGTA | 6894 |
| 1623 | UGGAAAAA G CUGUUUGG | 2843 | CCAAACAG GGCTAGCTACAACGA TTTTTCCA | 6895 |
| 1626 | AAAAAACU G UUUGGGAC | 2320 | GTCCCAAA GGCTAGCTACAACGA AGTTTTTT | 6896 |
| 1633 | UGUUUGGG G CCUCCGGU | 2844 | ACCGGAGG GGCTAGCTACAACGA CCCAAACA | 6897 |
| 1640 | GACCUCCG G UCAGAAAA | 2321 | TTTTCTGA GGCTAGCTACAACGA CGGAGGTC | 6898 |
| 1648 | GUCAGAAA G CCAAAAUU | 2845 | AATTTTGG GGCTAGCTACAACGA TTTCTGAC | 6899 |
| 1654 | AAACCAAA G UUAUAAGC | 2846 GCTTATAA GGCTAGCTACAACGA TTTGGTTT | 6900 | |
| 1657 | CCAAAAUU G UAAGCAAC | 209 | GTTGCTTA GGCTAGCTACAACGA AATTTTGG | 6901 |
| 1661 | AAUUAUAA G CAACAGAG | 2322 | CTCTGTTG GGCTAGCTACAACGA TTATAATT | 6902 |
| 1664 | UAUAAGCA G CAGAGGUG | 2847 | CACCTCTG GGCTAGCTACAACGA TGCTTATA | 6903 |
| 1670 | CAACAGAG G UGAAAACA | 2323 | TGTTTTCA GGCTAGCTACAACGA CTCTGTTG | 6904 |
| 1676 | AGGUGAAA G CAGCUGCA | 2848 | TGCAGCTG GGCTAGCTACAACGA TTTCACCT | 6905 |
| 1679 | UGAAAACA G CUGCAAGG | 2324 | CCTTGCAG GGCTAGCTACAACGA TGTTTTCA | 6906 |
| 1682 | AAACAGCU G CAAGGCCA | 2325 | TGGCCTTG GGCTAGCTACAACGA AGCTGTTT | 6907 |
| 1687 | GCUGCAAG G CCACAGGC | 2326 | GCCTGTGG GGCTAGCTACAACGA CTTGCAGC | 6908 |
| 1690 | GCAAGGCC G CAGGCCAG | 1200 | CTGGCCTG GGCTAGCTACAACGA GGCCTTGC | 6909 |
| 1694 | GGCCACAG G CCAGGUCU | 2327 | AGACCTGG GGCTAGCTACAACGA CTGTGGCC | 6910 |
| 1699 | CAGGCCAG G UCUGCCAU | 2328 | ATGGCAGA GGCTAGCTACAACGA CTGGCCTG | 6911 |
| 1703 | CCAGGUCU G CCAUGCCU | 2329 | AGGCATGG GGCTAGCTACAACGA AGACCTGG | 6912 |
| 1706 | GGUCUGCC G UGCCUUGU | 1206 | ACAAGGCA GGCTAGCTACAACGA GGCAGACC | 6913 |
| 1708 | UCUGCCAU G CCUUGUGC | 2330 | GCACAAGG GGCTAGCTACAACGA ATGGCAGA | 6914 |
| 1713 | CAUGCCUU G UGCUCCCC | 2331 | GGGGAGCA GGCTAGCTACAACGA AAGGCATG | 6915 |
| 1715 | UGCCUUGU G CUCCCCCG | 2332 | CGGGGGAG GGCTAGCTACAACGA ACAAGGCA | 6916 |
| 1727 | CCCCGAGG G CUGCUGGG | 2333 | CCCAGCAG GGCTAGCTACAACGA CCTCGGGG | 6917 |
| 1730 | CGAGGGCU G CUGGGGCC | 2334 | GGCCCCAG GGCTAGCTACAACGA AGCCCTCG | 6918 |
| 1736 | CUGCUGGG G CCCGGAGC | 2335 | GCTCCGGG GGCTAGCTACAACGA CCCAGCAG | 6919 |
| 1743 | GGCCCGGA G CCCAGGGA | 2336 | TCCCTGGG GGCTAGCTACAACGA TCCGGGCC | 6920 |
| 1751 | GCCCAGGG G CUGCGUCU | 2849 | AGACGCAG GGCTAGCTACAACGA CCCTGGGC | 6921 |
| 1754 | CAGGGACU G CGUCUCUU | 2337 | AAGAGACG GGCTAGCTACAACGA AGTCCCTG | 6922 |
| 1756 | GGGACUGC G UCUCUUGC | 2338 | GCAAGAGA GGCTAGCTACAACGA GCAGTCCC | 6923 |
| 1763 | CGUCUCUU G CCGGAAUG | 2339 | CATTCCGG GGCTAGCTACAACGA AAGAGACG | 6924 |
| 1769 | UUGCCGGA G UGUCAGCC | 2850 | GGCTGACA GGCTAGCTACAACGA TCCGGCAA | 6925 |
| 1771 | GCCGGAAU G UCAGCCGA | 2340 | TCGGCTGA GGCTAGCTACAACGA ATTCCGGC | 6926 |
| 1775 | GAAUGUCA G CCGAGGCA | 2341 | TGCCTCGG GGCTAGCTACAACGA TGACATTC | 6927 |
| 1781 | CAGCCGAG G CAGGGAAU | 2342 | ATTCCCTG GGCTAGCTACAACGA CTCGGCTG | 6928 |
| 1788 | GGCAGGGA G UGCGUGGA | 2851 | TCCACGCA GGCTAGCTACAACGA TCCCTGCC | 6929 |
| 1790 | CAGGGAAU G CGUGGACA | 2343 | TGTCCACG GGCTAGCTACAACGA ATTCCCTG | 6930 |
| 1792 | GGGAAUGC G UGGACAAG | 2344 | CTTGTCCA GGCTAGCTACAACGA GCATTCCC | 6931 |
| 1796 | AUGCGUGG G CAAGUGCA | 2852 | TGCACTTG GGCTAGCTACAACGA CCACGCAT | 6932 |
| 1800 | GUGGACAA G UGCAAGCU | 2345 | AGCTTGCA GGCTAGCTACAACGA TTGTCCAC | 6933 |
| 1802 | GGACAAGU G CAAGCUUC | 2346 | GAAGCTTG GGCTAGCTACAACGA ACTTGTCC | 6934 |
| 1806 | AAGUGCAA G CUUCUGGA | 2347 | TCCAGAAG GGCTAGCTACAACGA TTGCACTT | 6935 |
| 1817 | UCUGGAGG G UGAGCCAA | 2348 | TTGGCTCA GGCTAGCTACAACGA CCTCCAGA | 6936 |
| 1821 | GAGGGUGA G CCAAGGGA | 2349 | TCCCTTGG GGCTAGCTACAACGA TCACCCTC | 6937 |
| 1830 | CCAAGGGA G UUUGUGGA | 2350 | TCCACAAA GGCTAGCTACAACGA TCCCTTGG | 6938 |
| 1834 | GGGAGUUU G UGGAGAAC | 2351 | GTTCTCCA GGCTAGCTACAACGA AAACTCCC | 6939 |
| 1841 | UGUGGAGA G CUCUGAGU | 2853 | ACTCAGAG GGCTAGCTACAACGA TCTCCACA | 6940 |
| 1848 | AACUCUGA G UGCAUACA | 2352 | TGTATGCA GGCTAGCTACAACGA TCAGAGTT | 6941 |
| 1850 | CUCUGAGU G CAUACAGU | 2353 | ACTGTATG GGCTAGCTACAACGA ACTCAGAG | 6942 |
| 1852 | CUGAGUGC G UACAGUGC | 1236 | GCACTGTA GGCTAGCTACAACGA GCACTCAG | 6943 |
| 1854 | GAGUGCAU G CAGUOCCA | 223 | TGGCACTG GGCTAGCTACAACGA ATGCACTC | 6944 |
| 1857 | UGCAUACA G UGCCACCC | 2354 | GGGTGGCA GGCTAGCTACAACGA TGTATGCA | 6945 |
| 1859 | CAUACAGU G CCACCCAG | 2355 | CTGGGTGG GGCTAGCTACAACGA ACTGTATG | 6946 |
| 1862 | ACAGUGCC G CCCAGAGU | 1239 | ACTCTGGG GGCTAGCTACAACGA GGCACTGT | 6947 |
| 1869 | CACCCAGA G UGCCUGCC | 2356 | GGCAGGCA GGCTAGCTACAACGA TCTGGGTG | 6948 |
| 1871 | CCCAGAGU G CCUGCCUC | 2357 | GAGGCAGG GGCTAGCTACAACGA ACTCTGGG | 6949 |
| 1875 | GAGUGCCU G CCUCAGGC | 2358 | GCCTGAGG GGCTAGCTACAACGA AGGCACTC | 6950 |
| 1882 | UGCCUCAG G CCAUGAAC | 2359 | GTTCATGG GGCTAGCTACAACGA CTGAGGCA | 6951 |
| 1885 | CUCAGGCC G UGAACAUC | 1249 | GATGTTCA GGCTAGCTACAACGA GGCCTGAG | 6952 |
| 1889 | GGCCAUGA G CAUCACCU | 2854 | AGGTGATG GGCTAGCTACAACGA TCATGGCC | 6953 |
| 1891 | CCAUGAAC G UCACCUGC | 1250 | GCAGGTGA GGCTAGCTACAACGA GTTCATGG | 6954 |
| 1894 | UGAACAUC G CCUGCACA | 1251 | TGTGCAGG GGCTAGCTACAACGA GATGTTCA | 6955 |
| 1898 | CAUCACCU G CACAGGAC | 2360 | GTCCTGTG GGCTAGCTACAACGA AGGTGATG | 6956 |
| 1900 | UCACCUGC G CAGGACGG | 1254 | CCGTCCTG GGCTAGCTACAACGA GCAGGTGA | 6957 |
| 1905 | UGCACAGG G CGGGGACC | 2855 | GGTCCCCG GGCTAGCTACAACGA CCTGTGCA | 6958 |
| 1911 | GGACGGGG G CCAGACAA | 2856 | TTGTCTGG GGCTAGCTACAACGA CCCCGTCC | 6959 |
| 1916 | GGGACCAG G CAACUGUA | 2857 | TACAGTTG GGCTAGCTACAACGA CTGGTCCC | 6960 |
| 1919 | ACCAGACA G CUGUAUCC | 2858 | GGATACAG GGCTAGCTACAACGA TGTCTGGT | 6961 |
| 1922 | AGACAACU G UAUCCAGU | 2361 | ACTGGATA GGCTAGCTACAACGA AGTTGTCT | 6962 |
| 1924 | ACAACUGU G UCCAGUGU | 226 | ACACTGGA GGCTAGCTACAACGA ACAGTTGT | 6963 |
| 1929 | UGUAUCCA G UGUGCCCA | 2362 | TGGGCACA GGCTAGCTACAACGA TGGATACA | 6964 |
| 1931 | UAUCCAGU G UGCCCACU | 2363 | AGTGGGCA GGCTAGCTACAACGA ACTGGATA | 6965 |
| 1933 | UCCAGUGU G CCCACUAC | 2364 | GTAGTGGG GGCTAGCTACAACGA ACACTGGA | 6966 |
| 1937 | GUGUGCCC G CUACAUUG | 1264 | CAATGTAG GGCTAGCTACAACGA GGGCACAC | 6967 |
| 1940 | UGCCCACU G CAUUGACG | 228 | CGTCAATG GGCTAGCTACAACGA AGTGGGCA | 6968 |
| 1942 | CCCACUAC G TTGACGGC | 1266 | GCCGTCAA GGCTAGCTACAACGA GTAGTGGG | 6969 |
| 1946 | CUACAUUG G CGGCCCCC | 2859 | GGGGGCCG GGCTAGCTACAACGA CAATGTAG | 6970 |
| 1949 | CAUUGACG G CCCCCACU | 2365 | AGTGGGGG GGCTAGCTACAACGA CGTCAATG | 6971 |
| 1955 | CGGCCCCC G CUGCGUCA | 1271 | TGACGCAG GGCTAGCTACAACGA GGGGGCCG | 6972 |
| 1958 | CCCCCACU G CGUCAAGA | 2366 | TCTTGACG GGCTAGCTACAACGA AGTGGGGG | 6973 |
| 1960 | CCCACUGC G UCAAGACC | 2367 | GGTCTTGA GGCTAGCTACAACGA GCAGTGGG | 6974 |
| 1966 | GCGUCAAG G CCUGCCCG | 2860 | CGGGCAGG GGCTAGCTACAACGA CTTGACGC | 6973 |
| 1970 | CAAGACCU G CCCGGCAG | 2368 | CTGCCGGG GGCTAGCTACAACGA AGGTCTTG | 6976 |
| 1975 | CCUGCCCG G CAGGAGUC | 2369 | GACTCCTG GGCTAGCTACAACGA CGGGCAGG | 6977 |
| 1981 | CGGCAGGA G UCAUGGGA | 2370 | TCCCATGA GGCTAGCTACAACGA TCCTGCCG | 6978 |
| 1984 | CAGGAGUC G UGGGAGAA | 1279 | TTCTCCCA GGCTAGCTACAACGA GACTCCTG | 6979 |
| 1994 | GGGAGAAA G CAACACCC | 2861 | GGGTGTTG GGCTAGCTACAACGA TTTCTCCC | 6980 |
| 1997 | AGAAAACA G CACCCUGG | 2862 | CCAGGGTG GGCTAGCTACAACGA TGTTTTCT | 6981 |
| 1999 | AAAACAAC G CCCUGGUC | 1281 | GACCAGGG GGCTAGCTACAACGA GTTGTTTT | 6982 |
| 2005 | ACACCCUG G UCUGGAAG | 2371 | CTTCCAGA GGCTAGCTACAACGA CAGGGTGT | 6983 |
| 2013 | GUCUGGAA G UACGCAGA | 2372 | TCTGCGTA GGCTAGCTACAACGA TTCCAGAC | 6984 |
| 2015 | CUGGAAGU G CGCAGACG | 233 | CGTCTGCG GGCTAGCTACAACGA ACTTCCAG | 6985 |
| 2017 | GGAAGUAC G CAGACGCC | 2373 | GGCGTCTG GGCTAGCTACAACGA GTACTTCC | 6986 |
| 2021 | GUACGCAG G CGCCGGCC | 2863 | GGCCGGCG GGCTAGCTACAACGA CTGCGTAC | 6987 |
| 2023 | ACGCAGAC G CCGGCCAU | 2374 | ATGGCCGG GGCTAGCTACAACGA GTCTGCGT | 6988 |
| 2027 | AGACGCCG G CCAUGUGU | 2375 | ACACATGG GGCTAGCTACAACGA CGGCGTCT | 6989 |
| 2030 | CGCCGGCC G UGUGUGCC | 1289 | GGCACACA GGCTAGCTACAACGA GGCCGGCG | 6990 |
| 2032 | CCGGCCAU G UGUGCCAC | 2376 | GTGGCACA GGCTAGCTACAACGA ATGGCCGG | 6991 |
| 2034 | GGCCAUGU G UGCCACCU | 2377 | AGGTGGCA GGCTAGCTACAACGA ACATGGCC | 6992 |
| 2036 | CCAUGUGU G CCACCUGU | 2378 | ACAGGTGG GGCTAGCTACAACGA ACACATGG | 6993 |
| 2039 | UGUGUGCC G CCUGUGCC | 1291 | GGCACAGG GGCTAGCTACAACGA GGCACACA | 6994 |
| 2043 | UGCCACCU G UGCCAUCC | 2379 | GGATGGCA GGCTAGCTACAACGA AGGTGGCA | 6995 |
| 2045 | CCACCUGU G CCAUCCAA | 2380 | TTGGATGG GGCTAGCTACAACGA ACAGGTGG | 6996 |
| 2048 | CCUGUGCC G UCCAAACU | 1295 | AGTTTGGA GGCTAGCTACAACGA GGCACAGG | 6997 |
| 2054 | CCAUCCAA G CUGCACCU | 2864 | AGGTGCAG GGCTAGCTACAACGA TTGGATGG | 6998 |
| 2057 | UCCAAACU G CACCUACG | 2381 | CGTAGGTG GGCTAGCTACAACGA AGTTTGGA | 6999 |
| 2059 | CAAACUGC G CCUACGGA | 1299 | TCCGTAGG GGCTAGCTACAACGA GCAGTTTG | 7000 |
| 2063 | CUGCACCU G CGGAUGCA | 235 | TGCATCCG GGCTAGCTACAACGA AGGTGCAG | 7001 |
| 2067 | ACCUACGG G UGCACUGG | 2865 | CCAGTGCA GGCTAGCTACAACGA CCGTAGGT | 7002 |
| 2069 | CUACGGAU G CACUGGGC | 2382 | GCCCAGTG GGCTAGCTACAACGA ATCCGTAG | 7003 |
| 2071 | ACGGAUGC G CUGGGCCA | 1302 | TGGCCCAG GGCTAGCTACAACGA GCATCCGT | 7004 |
| 2076 | UGCACUGG G CCAGGUCU | 2383 | AGACCTGG GGCTAGCTACAACGA CCAGTGCA | 7005 |
| 2081 | UGGGCCAG G UCUUGAAG | 2384 | CTTCAAGA GGCTAGCTACAACGA CTGGCCCA | 7006 |
| 2090 | UCUUGAAG G CUGUCCAA | 2385 | TTGGACAG GGCTAGCTACAACGA CTTCAAGA | 7007 |
| 2093 | UGAAGGCU G UCCAACGA | 2386 | TCGTTGGA GGCTAGCTACAACGA AGCCTTCA | 7008 |
| 2098 | GCUGUCCA G CGAAUGGG | 2866 | CCCATTCG GGCTAGCTACAACGA TGGACAGC | 7009 |
| 2102 | UCCAACGA G UGGGCCUA | 2867 | TAGGCCCA GGCTAGCTACAACGA TCGTTGGA | 7010 |
| 2106 | ACGAAUGG G CCUAAGAU | 2387 | ATCTTAGG GGCTAGCTACAACGA CCATTCGT | 7011 |
| 2113 | GGCCUAAG G UCCCGUCC | 2868 | GGACGGGA GGCTAGCTACAACGA CTTAGGCC | 7012 |
| 2118 | AAGAUCCC G UCCAUCGC | 2388 | GCGATGGA GGCTAGCTACAACGA GGGATCTT | 7013 |
| 2122 | UCCCGUCC G UCGCCACU | 1315 | AGTGGCGA GGCTAGCTACAACGA GGACGGGA | 7014 |
| 2125 | CGUCCAUC G CCACUGGG | 2389 | CCCAGTGG GGCTAGCTACAACGA GATGGACG | 7015 |
| 2128 | CCAUCGCC G CUGGGAUG | 1317 | CATCCCAG GGCTAGCTACAACGA GGCGATGG | 7016 |
| 2134 | CCACUGGG G UGGUGGGG | 2869 | CCCCACCA GGCTAGCTACAACGA CCCAGTGG | 7017 |
| 2137 | CUGGGAUG G UGGGGGCC | 2390 | GGCCCCCA GGCTAGCTACAACGA CATCCCAG | 7018 |
| 2143 | UGGUGGGG G CCCUCCUC | 2391 | GAGGAGGG GGCTAGCTACAACGA CCCCACCA | 7019 |
| 2154 | CUCCUCUU G CUGCUGGU | 2392 | ACCAGCAG GGCTAGCTACAACGA AAGAGGAG | 7020 |
| 2157 | CUCUUGCU G CUGGUGGU | 2393 | ACCACCAG GGCTAGCTACAACGA AGCAAGAG | 7021 |
| 2161 | UGCUGCUG G UGGUGGCC | 2394 | GGCCACCA GGCTAGCTACAACGA CAGCAGCA | 7022 |
| 2164 | UGCUGGUG G UGGCCCUG | 2395 | CAGGGCCA GGCTAGCTACAACGA CACCAGCA | 7023 |
| 2167 | UGGUGGUG G CCCUGGGG | 2396 | CCCCAGGG GGCTAGCTACAACGA CACCACCA | 7024 |
| 2180 | GGGGAUCG G CCUCUUCA | 2397 | TGAAGAGG GGCTAGCTACAACGA CGATCCCC | 7026 |
| 2188 | GCCUCUUC G UGCGAAGG | 1333 | CCTTCGCA GGCTAGCTACAACGA GAAGAGGC | 7027 |
| 2190 | CUCUUCAU G CGAAGGCG | 2398 | CGCCTTCG GGCTAGCTACAACGA ATGAAGAG | 7028 |
| 2196 | AUGCGAAG G CGCCACAU | 2399 | ATGTGGCG GGCTAGCTACAACGA CTTCGCAT | 7029 |
| 2198 | GCGAAGGC G CCACAUCG | 2400 | CGATGTGG GGCTAGCTACAACGA GCCTTCGC | 7030 |
| 2201 | AAGGCGCC G CAUCGUUC | 1335 | GAACGATG GGCTAGCTACAACGA GGCGCCTT | 7031 |
| 2203 | GGCGCCAC G UCGUUCGG | 1336 | CCGAACGA GGCTAGCTACAACGA GTGGCGCC | 7032 |
| 2206 | GCCACAUC G UUCGGAAG | 2401 | CTTCCGAA GGCTAGCTACAACGA GATGTGGC | 7033 |
| 2214 | GUUCGGAA G CGCACGCU | 2402 | AGCGTGCG GGCTAGCTACAACGA TTCCGAAC | 7034 |
| 2216 | UCGGAAGC G CACGCUGC | 2403 | GCAGCGTG GGCTAGCTACAACGA GCTTCCGA | 7035 |
| 2218 | GGAAGCGC G CGCUGCGG | 1337 | CCGCAGCG GGCTAGCTACAACGA GCGCTTCC | 7036 |
| 2220 | AAGCGCAC G CUGCGGAG | 2404 | CTCCGCAG GGCTAGCTACAACGA GTGCGCTT | 7037 |
| 2223 | CGCACGCU G CGGAGGCU | 2405 | AGCCTCCG GGCTAGCTACAACGA AGCGTGCG | 7038 |
| 2229 | CUGCGGAG G CUGCUGCA | 2406 | TGCAGCAG GGCTAGCTACAACGA CTCCGCAG | 7039 |
| 2232 | CGGAGGCU G CUGCAGGA | 2407 | TCCTGCAG GGCTAGCTACAACGA AGCCTCCG | 7040 |
| 2235 | AGGCUGCU G CAGGAGAG | 2408 | CTCTCCTG GGCTAGCTACAACGA AGCAGCCT | 7041 |
| 2247 | GAGAGGGA G CUUGUGGA | 2409 | TCCACAAG GGCTAGCTACAACGA TCCCTCTC | 7042 |
| 2251 | GGGAGCUU G UGGAGCCU | 2410 | AGGCTCCA GGCTAGCTACAACGA AAGCTCCC | 7043 |
| 2256 | CUUGUGGA G CCUCUUAC | 2411 | GTAAGAGG GGCTAGCTACAACGA TCCACAAG | 7044 |
| 2263 | AGCCUCUU G CACCCAGU | 256 | ACTGGGTG GGCTAGCTACAACGA AAGAGGCT | 7045 |
| 2265 | CCUCUUAC G CCCAGUGG | 1346 | CCACTGGG GGCTAGCTACAACGA GTAAGAGG | 7046 |
| 2270 | UACACCCA G UGGAGAAG | 2412 | CTTCTCCA GGCTAGCTACAACGA TGGGTGTA | 7047 |
| 2278 | GUGGAGAA G CUCCCAAC | 2413 | GTTGGGAG GGCTAGCTACAACGA TTCTCCAC | 7048 |
| 2285 | AGCUCCCA G CCAAGCUC | 2871 | GAGCTTGG GGCTAGCTACAACGA TGGGAGCT | 7049 |
| 2290 | CCAACCAA G CUCUCUUG | 2414 | CAAGAGAG GGCTAGCTACAACGA TTGGTTGG | 7050 |
| 2302 | UCUUGAGG G UCUUGAAG | 2872 | CTTCAAGA GGCTAGCTACAACGA CCTCAAGA | 7051 |
| 2314 | UGAAGGAA G CUGAAUUC | 2873 | GAATTCAG GGCTAGCTACAACGA TTCCTTCA | 7052 |
| 2319 | GAAACUGA G UUCAAAAA | 2874 | TTTTTGAA GGCTAGCTACAACGA TCAGTTTC | 7053 |
| 2329 | UCAAAAAG G UCAAAGUG | 2875 | CACTTTGA GGCTAGCTACAACGA CTTTTTGA | 7054 |
| 2335 | AGAUCAAA G UGCUGGGC | 2415 | GCCCAGCA GGCTAGCTACAACGA TTTGATCT | 7055 |
| 2337 | AUCAAAGU G CUGGGCUC | 2416 | GAGCCCAG GGCTAGCTACAACGA ACTTTGAT | 7056 |
| 2342 | AGUGCUGG G CUCCGGUG | 2417 | CACCGGAG GGCTAGCTACAACGA CCAGCACT | 7057 |
| 2348 | GGGCUCCG G UGCGUUCG | 2418 | CGAACGCA GGCTAGCTACAACGA CGGAGCCC | 7058 |
| 2350 | GCUCCGGU G CGUUCGGC | 2419 | GCCGAACG GGCTAGCTACAACGA ACCGGAGC | 7059 |
| 2352 | UCCGGUGC G UUCGGCAC | 2420 | GTGCCGAA GGCTAGCTACAACGA GCACCGGA | 7060 |
| 2357 | UGCGUUCG G CACGGUGU | 2421 | ACACCGTG GGCTAGCTACAACGA CGAACGCA | 7061 |
| 2359 | CGUUCGGC G CGGUGUAU | 1366 | ATACACCG GGCTAGCTACAACGA GCCGAACG | 7062 |
| 2362 | UCGGCACG G UGUAUAAG | 2422 | CTTATACA GGCTAGCTACAACGA CGTGCCGA | 7063 |
| 2364 | GGCACGGU G UAUAAGGG | 2423 | CCCTTATA GGCTAGCTACAACGA ACCGTGCC | 7064 |
| 2366 | CACGGUGU G UAAGGGAC | 269 | GTCCCTTA GGCTAGCTACAACGA ACACCGTG | 7065 |
| 2373 | UAUAAGGG G CUCUGGAU | 2876 | ATCCAGAG GGCTAGCTACAACGA CCCTTATA | 7066 |
| 2380 | GACUCUGG G UCCCAGAA | 2877 | TTCTGGGA GGCTAGCTACAACGA CCAGAGTC | 7067 |
| 2390 | CCCAGAAG G UGAGAAAG | 2424 | CTTTCTCA GGCTAGCTACAACGA CTTCTGGG | 7068 |
| 2398 | GUGAGAAA G UUAAAAUU | 2425 | AATTTTAA GGCTAGCTACAACGA TTTCTCAC | 7069 |
| 2404 | AAGUUAAA G UUCCCGUC | 2878 | GACGGGAA GGCTAGCTACAACGA TTTAACTT | 7070 |
| 2410 | AAAUUCCC G UCGCUAUC | 2426 | GATAGCGA GGCTAGCTACAACGA GGGAATTT | 7071 |
| 2413 | UUCCCGUC G CUAUCAAG | 2427 | CTTGATAG GGCTAGCTACAACGA GACGGGAA | 7072 |
| 2416 | CCGUCGCU G UCAAGGAA | 278 | TTCCTTGA GGCTAGCTACAACGA AGCGACGG | 7073 |
| 2424 | AUCAAGGA G UUAAGAGA | 2879 | TCTCTTAA GGCTAGCTACAACGA TCCTTGAT | 7074 |
| 2434 | UAAGAGAA G CAACAUCU | 2428 | AGATGTTG GGCTAGCTACAACGA TTCTCTTA | 7075 |
| 2437 | GAGAAGCA G CAUCUCCG | 2880 | CGGAGATG GGCTAGCTACAACGA TGCTTCTC | 7076 |
| 2439 | GAAGCAAC G UCUCCGAA | 1377 | TTCGGAGA GGCTAGCTACAACGA GTTGCTTC | 7077 |
| 2449 | CUCCGAAA G CCAACAAG | 2429 | CTTGTTGG GGCTAGCTACAACGA TTTCGGAG | 7078 |
| 2453 | GAAAGCCA G CAAGGAAA | 2881 | TTTCCTTG GGCTAGCTACAACGA TGGCTTTC | 7079 |
| 2461 | ACAAGGAA G UCCUCGAU | 2882 | ATCGAGGA GGCTAGCTACAACGA TTCCTTGT | 7080 |
| 2468 | AAUCCUCG G UGAAGCCU | 2883 | AGGCTTCA GGCTAGCTACAACGA CGAGGATT | 7081 |
| 2473 | UCGAUGAA G CCUACGUG | 2430 | CACGTAGG GGCTAGCTACAACGA TTCATCGA | 7082 |
| 2477 | UGAAGCCU G CGUGAUGG | 286 | CCATCACG GGCTAGCTACAACGA AGGCTTCA | 7083 |
| 2479 | AAGCCUAC G UGAUGGCC | 2431 | GGCCATCA GGCTAGCTACAACGA GTAGGCTT | 7084 |
| 2482 | CCUACGUG G UGGCCAGC | 2884 | GCTGGCCA GGCTAGCTACAACGA CACGTAGG | 7085 |
| 2485 | ACGUGAUG G CCAGCGUG | 2432 | CACGCTGG GGCTAGCTACAACGA CATCACGT | 7086 |
| 2489 | GAUGGCCA G CGUGGACA | 2433 | TGTCCACG GGCTAGCTACAACGA TGGCCATC | 7087 |
| 2491 | UGGCCAGC G UGGACAAC | 2434 | GTTGTCCA GGCTAGCTACAACGA GCTGGCCA | 7088 |
| 2495 | CAGCGUGG G CAACCCCC | 2885 | GGGGGTTG GGCTAGCTACAACGA CCACGCTG | 7089 |
| 2498 | CGUGGACA G CCCCCACG | 2886 | CGTGGGGG GGCTAGCTACAACGA TGTCCACG | 7090 |
| 2504 | CAACCCCC G CGUGUGCC | 1394 | GGCACACG GGCTAGCTACAACGA GGGGGTTG | 7091 |
| 2506 | ACCCCCAC G UGUGCCGC | 2435 | GCGGCACA GGCTAGCTACAACGA GTGGGGGT | 7092 |
| 2508 | CCCCACGU G UGCCGCCU | 2436 | AGGCGGCA GGCTAGCTACAACGA ACGTGGGG | 7093 |
| 2510 | CCACGUGU G CCGCCUGC | 2437 | GCAGGCGG GGCTAGCTACAACGA ACACGTGG | 7094 |
| 2513 | CGUGUGCC G CCUGCUGG | 2438 | CCAGCAGG GGCTAGCTACAACGA GGCACACG | 7095 |
| 2517 | UGCCGCCU G CUGGGCAU | 2439 | ATGCCCAG GGCTAGCTACAACGA AGGCGGCA | 7096 |
| 2522 | CCUGCUGG G CAUCUGCC | 2440 | GGCAGATG GGCTAGCTACAACGA CCAGCAGG | 7097 |
| 2524 | UGCUGGGC G UCUGCCUC | 1399 | GAGGCAGA GGCTAGCTACAACGA GCCCAGCA | 7098 |
| 2528 | GGGCAUCU G CCUCACCU | 2441 | AGGTGAGG GGCTAGCTACAACGA AGATGCCC | 7099 |
| 2533 | UCUGCCUC G CCUCCACC | 1403 | GGTGGAGG GGCTAGCTACAACGA GAGGCAGA | 7100 |
| 2539 | UCACCUCC G CCGUGCAA | 1407 | TTGCACGG GGCTAGCTACAACGA GGAGGTGA | 7101 |
| 2542 | CCUCCACC G UGCAACUC | 2442 | GAGTTGCA GGCTAGCTACAACGA GGTGGAGG | 7102 |
| 2544 | UCCACCGU G CAACUCAU | 2443 | ATGAGTTG GGCTAGCTACAACGA ACGGTGGA | 7103 |
| 2547 | ACCGUGCA G CUCAUCAC | 2887 | GTGATGAG GGCTAGCTACAACGA TGCACGGT | 7104 |
| 2551 | UGCAACUC G UCACGCAG | 1411 | CTGCGTGA GGCTAGCTACAACGA GAGTTGCA | 7105 |
| 2554 | AACUCAUC G CGCAGCUC | 1412 | GAGCTGCG GGCTAGCTACAACGA GATGAGTT | 7106 |
| 2556 | CUCAUCAC G CAGCUCAU | 2444 | ATGAGCTG GGCTAGCTACAACGA GTGATGAG | 7107 |
| 2559 | AUCACGCA G CUCAUGCC | 2445 | GGCATGAG GGCTAGCTACAACGA TGCGTGAT | 7108 |
| 2563 | CGCAGCUC G UGCCCUUC | 1415 | GAAGGGCA GGCTAGCTACAACGA GAGCTGCG | 7109 |
| 2565 | CAGCUCAU G CCCUUCGG | 2446 | CCGAAGGG GGCTAGCTACAACGA ATGAGCTG | 7110 |
| 2573 | GCCCUUCG G CUGCCUCC | 2447 | GGAGGCAG GGCTAGCTACAACGA CGAAGGGC | 7111 |
| 2576 | CUUCGGCU G CCUCCUGG | 2448 | CCAGGAGG GGCTAGCTACAACGA AGCCGAAG | 7112 |
| 2585 | CCUCCUGG G CUAUGUCC | 2888 | GGACATAG GGCTAGCTACAACGA CCAGGAGG | 7113 |
| 2588 | CCUGGACU G UGUCCGGG | 296 | CCCGGACA GGCTAGCTACAACGA AGTCCAGG | 7114 |
| 2590 | UGGACUAU G UCCGGGAA | 2449 | TTCCCGGA GGCTAGCTACAACGA ATAGTCCA | 7115 |
| 2598 | GUCCGGGA G CACAAAGA | 2889 | TCTTTGTG GGCTAGCTACAACGA TCCCGGAC | 7116 |
| 2600 | CCGGGAAC G CAAAGACA | 1426 | TGTCTTTG GGCTAGCTACAACGA GTTCCCGG | 7117 |
| 2606 | ACACAAAG G CAAUAUUG | 2890 | CAATATTG GGCTAGCTACAACGA CTTTGTGT | 7118 |
| 2609 | CAAAGACA G UAUUGGCU | 2891 | AGCCAATA GGCTAGCTACAACGA TGTCTTTG | 7119 |
| 2611 | AAGACAAU G UUGGCUCC | 298 | GGAGCCAA GGCTAGCTACAACGA ATTGTCTT | 7120 |
| 2615 | CAAUAUUG G CUCCCAGU | 2450 | ACTGGGAG GGCTAGCTACAACGA CAATATTG | 7121 |
| 2622 | GGCUCCCA G UACCUCCU | 2451 | AGCAGGTA GGCTAGCTACAACGA TGGGAGCC | 7122 |
| 2624 | CUCCCAGU G CCUGCUCA | 301 | TGAGCAGG GGCTAGCTACAACGA ACTGGGAG | 7123 |
| 2628 | CAGUACCU G CUCAACUG | 2452 | CAGTTGAG GGCTAGCTACAACGA AGGTACTG | 7124 |
| 2633 | CCUGCUCA G CUGGUGUG | 2892 | CACACCAG GGCTAGCTACAACGA TGAGCAGG | 7125 |
| 2637 | CUCAACUG G UGUGUGCA | 2453 | TGCACACA GGCTAGCTACAACGA CAGTTGAG | 7126 |
| 2639 | CAACUGGU G UGUGCAGA | 2454 | TCTGCACA GGCTAGCTACAACGA ACCAGTTG | 7127 |
| 2641 | ACUGGUGU G UGCAGAUC | 2455 | GATCTGCA GGCTAGCTACAACGA ACACCAGT | 7128 |
| 2643 | UGGUGUGU G CAGAUCGC | 2456 | GCGATCTG GGCTAGCTACAACGA ACACACCA | 7129 |
| 2647 | GUGUGCAG G UCGCAAAG | 2893 | CTTTGCGA GGCTAGCTACAACGA CTGCACAC | 7130 |
| 2650 | UGCAGAUC G CAAAGGGC | 2457 | GCCCTTTG GGCTAGCTACAACGA GATCTGCA | 7131 |
| 2657 | CGCAAAGG G CAUGAACU | 2458 | AGTTCATG GGCTAGCTACAACGA CCTTTGCG | 7132 |
| 2659 | CAAAGGGC G UGAACUAC | 1440 | GTAGTTCA GGCTAGCTACAACGA GCCCTTTG | 7133 |
| 2663 | GGGCAUGA G CUACUUGG | 2894 | CCAAGTAG GGCTAGCTACAACGA TCATGCCC | 7134 |
| 2666 | CAUGAACU G CUUGGAGG | 304 | CCTCCAAG GGCTAGCTACAACGA AGTTCATG | 7135 |
| 2675 | CUUGGAGG G CCGUCGCU | 2895 | AGCGACGG GGCTAGCTACAACGA CCTCCAAG | 7136 |
| 2678 | GGAGGACC G UCGCUUGG | 2459 | CCAAGCGA GGCTAGCTACAACGA GGTCCTCC | 7137 |
| 2681 | GGACCGUC G CUUGGUGC | 2460 | GCACCAAG GGCTAGCTACAACGA GACGGTCC | 7138 |
| 2686 | GUCGCUUG G UGCACCGC | 2461 | GCGGTGCA GGCTAGCTACAACGA CAAGCGAC | 7139 |
| 2688 | CGCUUGGU G CACCGCGA | 2462 | TCGCGGTG GGCTAGCTACAACGA ACCAAGCG | 7140 |
| 2690 | CUUGGUGC G CCGCGACC | 1445 | GGTCGCGG GGCTAGCTACAACGA GCACCAAG | 7141 |
| 2693 | GGUGCACC G CGACCUGG | 2463 | CCAGGTCG GGCTAGCTACAACGA GGTCCACC | 7142 |
| 2696 | GCACCGCG G CCUGGCAG | 2896 | CTGCCAGG GGCTAGCTACAACGA CGCGGTGC | 7143 |
| 2701 | GCGACCUG G CAGCCAGG | 2464 | CCTGCCTG GGCTAGCTACAACGA CACGTCGC | 7144 |
| 2704 | ACCUCGCA G CCAGGAAC | 2465 | GTTCCTGG GGCTAGCTACAACGA TGCCAGGT | 7145 |
| 2711 | AGCCAGGA G CGUACUGG | 2897 | CCAGTACG GGCTAGCTACAACGA TCCTGGCT | 7146 |
| 2713 | CCAGGAAC G UACUGGUG | 2466 | CACCAGTA GCCTAGCTACAACGA GTTCCTGG | 7147 |
| 2715 | AGGAACCU G CUGGUGAA | 308 | TTCACCAC GGCTAGCTACAACCA ACGTTCCT | 7148 |
| 2719 | ACGUACUG G UGAAAACA | 2467 | TGTTTTCA GGCTAGCTACAACGA CACTACGT | 7149 |
| 2725 | UGCUGAAA G CACCGCAC | 2898 | CTCCCCTC GGCTACCTACAACGA TTTCACCA | 7150 |
| 2727 | GUCAAAAC G CCGCAGCA | 1453 | TGCTGCGG GGCTAGCTACAACCA GTTTTCAC | 7151 |
| 2730 | AAAACACC G CAGCAUGU | 2468 | ACATGCTG GGCTAGCTACAACCA CCTGTTTT | 7152 |
| 2733 | ACACCGCA G CAUCUCAA | 2469 | TTGACATG GGCTAGCTACAACGA TGCGGTGT | 7153 |
| 2735 | ACCGCAGC G UGUCAAGA | 1456 | TCTTGACA GGCTACCTACAACGA GCTGCGGT | 7154 |
| 2737 | CGCACCAU G UCAAGAUC | 2470 | GATCTTGA GGCTAGCTACAACGA ATGCTGCC | 7155 |
| 2743 | AUGUCAAC G UCACAGAU | 2899 | ATCTGTCA GGCTAGCTACAACGA CTTGACAT | 7156 |
| 2746 | UCAACAUC G CAGAUUUU | 1458 | AAAATCTC GGCTACCTACAACGA GATCTTGA | 7157 |
| 2750 | GAUCACAG G UUUUCCCC | 2900 | GCCCAAAA GGCTAGCTACAACGA CTGTGATC | 7158 |
| 2757 | GAUUUUGC G CUCGCCAA | 2471 | TTGCCCAG GGCTAGCTACAACGA CCAAAATC | 7159 |
| 2761 | UUGGCCUG G CCAAACUG | 2472 | CAGTTTGG GGCTACCTACAACGA CAGCCCAA | 7160 |
| 2766 | CUGGCCAA G CUGCUGGG | 2901 | CCCAGCAG GGCTAGCTACAACCA TTCCCCAG | 7161 |
| 2769 | GCCAAACU G CUCCCUGC | 2473 | GCACCCAC GGCTAGCTACAACGA AGTTTGGC | 7162 |
| 2774 | ACUGCUGG G UGCGGAAC | 2474 | CTTCCGCA GGCTAGCTACAACGA CCAGCAGT | 7163 |
| 2776 | UGCUGGGU G CGCAAGAG | 2475 | CTCTTCCG GGCTAGCTACAACGA ACCCAGCA | 7164 |
| 2790 | CACAAACA G UACCAUCC | 2902 | GCATGGTA GGCTAGCTACAACGA TCTTTCTC | 7165 |
| 2792 | CAAACAAU G CCAUCCAC | 314 | CTCCATCC GGCTACCTACAACGA ATTCTTTC | 7166 |
| 2795 | AGAAUACC G UGCACAAC | 1466 | CTTCTCCA GGCTAGCTACAACGA GGTATTCT | 7167 |
| 2797 | AAUACCAU G CACAAGGA | 2476 | TCCTTCTG GGCTAGCTACAACGA ATGGTATT | 7168 |
| 2807 | ACAAGCAG G CAAACUGC | 2477 | CCACTTTC GGCTAGCTACAACGA CTCCTTCT | 7169 |
| 2812 | CAGCCAAA G UCCCUAUC | 2478 | CATACCCA GGCTACCTACAACGA TTTCCCTC | 7170 |
| 2814 | CCCAAACU G CCUAUCAA | 2479 | TTCATACC GGCTACCTACAACGA ACTTTCCC | 7171 |
| 2818 | AACUCCCU G UCAACUCC | 315 | CCACTTCA GGCTAGCTACAACGA ACCCACTT | 7172 |
| 2823 | CCUAUCAA G UCCAUGCC | 2480 | CCCATCCA GGCTACCTACAACGA TTCATACC | 7173 |
| 2827 | UCAACUCC G UCCCAUUG | 2903 | CAATGCCA GGCTACCTACAACGA CCACTTCA | 7174 |
| 2830 | AGUGGAUC G CAUUGGAA | 2481 | TTCCAATG GGCTAGCTACAACGA CATCCACT | 7175 |
| 2832 | UCCAUCCC G UUGGAAUC | 1472 | GATTCCAA GCCTACCTACAACCA CCCATCCA | 7176 |
| 2838 | GCAUUCCA G UCAAUUUU | 2904 | AAAATTGA GGCTAGCTACAACGA TCCAATCC | 7177 |
| 2842 | UGGAAUCA G UUUUACAC | 2905 | CTCTAAAA GGCTAGCTACAACGA TCATTCCA | 7178 |
| 2847 | UCAAUUUU G CACAGAAU | 322 | ATTCTGTG GGCTAGCTACAACGA AAAATTGA | 7179 |
| 2849 | AAUUUUAC G CAGAAUCU | 1474 | AGATTCTG GGCTAGCTACAACGA GTAAAATT | 7180 |
| 2854 | UACACAGA G UCUAUACC | 2906 | GGTATAGA GGCTAGCTACAACGA TCTGTGTA | 7181 |
| 2858 | CAGAAUCU G UACCCACC | 324 | GGTGGGTA GGCTAGCTACAACGA AGATTCTG | 7182 |
| 2860 | GAAUCUAU G CCCACCAG | 325 | CTGGTGGG GGCTAGCTACAACGA ATAGATTC | 7183 |
| 2864 | CUAUACCC G CCAGAGUG | 1479 | CACTCTGG GGCTAGCTACAACGA GGGTATAG | 7184 |
| 2870 | CCACCAGA G UGAUGUCU | 2482 | AGACATCA GGCTAGCTACAACGA TCTGGTGG | 7185 |
| 2873 | CCAGAGUG G UGUCUGGA | 2907 | TCCAGACA GGCTAGCTACAACGA CACTCTGG | 7186 |
| 2875 | AGAGUGAU G UCUGGAGC | 2483 | GCTCCAGA GGCTAGCTACAACGA ATCACTCT | 7187 |
| 2882 | UGUCUGGA G CUACGGGG | 2484 | CCCCGTAG GGCTAGCTACAACGA TCCAGACA | 7188 |
| 2885 | CUGGAGCU G CGGGGUGA | 327 | TCACCCCG GGCTAGCTACAACGA AGCTCCAG | 7189 |
| 2890 | GCUACGGG G UGACCGUU | 2485 | AACGGTCA GGCTAGCTACAACGA CCCGTAGC | 7190 |
| 2893 | ACGGGGUG G CCGUUUGG | 2908 | CCAAACGG GGCTAGCTACAACGA CACCCCGT | 7191 |
| 2896 | GGGUGACC G UUUGGGAG | 2486 | CTCCCAAA GGCTAGCTACAACGA GGTCACCC | 7192 |
| 2904 | GUUUGGGA G UUGAUGAC | 2487 | GTCATCAA GGCTAGCTACAACGA TCCCAAAC | 7193 |
| 2908 | GGGAGUUG G UGACCUUU | 2909 | AAAGGTCA GGCTAGCTACAACGA CAACTCCC | 7194 |
| 2911 | AGUUGAUG G CCUUUGGA | 2910 | TCCAAAGG GGCTAGCTACAACGA CATCAACT | 7195 |
| 2919 | ACCUUUGG G UCCAAGCC | 2911 | GGCTTGGA GGCTAGCTACAACGA CCAAAGGT | 7196 |
| 2925 | GGAUCCAA G CCAUAUGA | 2488 | TCATATGG GGCTAGCTACAACGA TTCGATCC | 7197 |
| 2928 | UCCAAGCC G UAUGACGG | 1490 | CCGTCATA GGCTAGCTACAACGA GGCTTGGA | 7198 |
| 2930 | CAAGCCAU G UGACGGAA | 334 | TTCCGTCA GGCTAGCTACAACGA ATGGCTTG | 7199 |
| 2933 | GCCAUAUG G CGGAAUCC | 2912 | GGATTCCG GGCTAGCTACAACGA CATATGGC | 7200 |
| 2938 | AUGACGGA G UCCCUGCC | 2913 | GGCAGGGA GGCTAGCTACAACGA TCCGTCAT | 7201 |
| 2944 | GAAUCCCU G CCAGCGAG | 2489 | CTCGCTGG GGCTAGCTACAACGA AGGGATTC | 7202 |
| 2948 | CCCUGCCA G CGAGAUCU | 2490 | AGATCTCG GGCTAGCTACAACGA TGGCAGGG | 7203 |
| 2953 | CCAGCGAG G UCUCCUCC | 2914 | GGAGGAGA GGCTAGCTACAACGA CTCGCTGG | 7204 |
| 2962 | UCUCCUCC G UCCUGGAG | 1500 | CTCCAGGA GGCTAGCTACAACGA GGAGGAGA | 7205 |
| 2979 | AAAGGAGA G CGCCUCCC | 2915 | GGGAGGCG GGCTAGCTACAACGA TCTCCTTT | 7206 |
| 2981 | AGGAGAAC G CCUCCCUC | 2491 | GAGGGAGG GGCTAGCTACAACGA GTTCTCCT | 7207 |
| 2991 | CUCCCUCA G CCACCCAU | 2492 | ATGGGTGG GGCTAGCTACAACGA TGAGGGAG | 7208 |
| 2994 | CCUCAGCC G CCCAUAUG | 1510 | CATATGGG GGCTAGCTACAACGA GGCTGAGG | 7209 |
| 2998 | AGCCACCC G UAUGUACC | 1513 | GGTACATA GGCTAGCTACAACGA GGGTGGCT | 7210 |
| 3000 | CCACCCAU G UGUACCAU | 342 | ATGGTACA GGCTAGCTACAACGA ATGGGTGG | 7211 |
| 3002 | ACCCAUAU G UACCAUCG | 2493 | CGATGGTA GGCTAGCTACAACGA ATATGGGT | 7212 |
| 3004 | CCAUAUGU G CCAUCGAU | 343 | ATCGATGG GGCTAGCTACAACGA ACATATGG | 7213 |
| 3007 | UAUGUACC G UCGAUGUC | 1515 | GACATCGA GGCTAGCTACAACGA GGTACATA | 7214 |
| 3011 | UACCAUCG G UGUCUACA | 2916 | TGTAGACA GGCTAGCTACAACGA CGATGGTA | 7215 |
| 3013 | CCAUCGAU G UCUACAUG | 2494 | CATGTAGA GGCTAGCTACAACGA ATCGATGG | 7216 |
| 3017 | CGAUGUCU G CAUGAUCA | 346 | TGATCATG GGCTAGCTACAACGA AGACATCG | 7217 |
| 3019 | AUGUCUAC G UGAUCAUG | 1517 | CATGATCA GGCTAGCTACAACGA GTAGACAT | 7218 |
| 3022 | UCUACAUG G UCAUGGUC | 2917 | GACCATGA GGCTAGCTACAACGA CATGTAGA | 7219 |
| 3025 | ACAUGAUC G UGGUCAAG | 1518 | CTTGACCA GGCTAGCTACAACGA GATCATGT | 7220 |
| 3028 | UGAUCAUG G UCAAGUGC | 2495 | GCACTTGA GGCTAGCTACAACGA CATGATCA | 7221 |
| 3033 | AUGGUCAA G UGCUGGAU | 2496 | ATCCAGCA GGCTAGCTACAACGA TTGACCAT | 7222 |
| 3035 | GGUCAAGU G CUGGAUGA | 2497 | TCATCCAG GGCTAGCTACAACGA ACTTGACC | 7223 |
| 3040 | AGUGCUGG G UGAUAGAC | 2918 | GTCTATCA GGCTAGCTACAACGA CCAGCACT | 7224 |
| 3043 | GCUGGAUG G UAGACGCA | 2919 | TGCGTCTA GGCTAGCTACAACGA CATCCAGC | 7225 |
| 3047 | GAUGAUAG G CGCAGAUA | 2920 | TATCTGCG GGCTAGCTACAACGA CTATCATC | 7226 |
| 3049 | UGAUAGAC G CAGAUAGU | 2498 | ACTATCTG GGCTAGCTACAACGA GTCTATCA | 7227 |
| 3053 | AGACGCAG G UAGUCGCC | 2921 | GGCGACTA GGCTAGCTACAACGA CTGCGTCT | 7228 |
| 3056 | CGCAGAUA G UCGCCCAA | 2499 | TTGGGCGA GGCTAGCTACAACGA TATCTGCG | 7229 |
| 3059 | AGAUAGUC G CCCAAAGU | 2500 | ACTTTGGG GGCTAGCTACAACGA GACTATCT | 7230 |
| 3066 | CGCCCAAA G UUCCGUGA | 2501 | TCACGGAA GGCTAGCTACAACGA TTTGGGCG | 7231 |
| 3071 | AAAGUUCC G UGAGUUGA | 2502 | TCAACTCA GGCTAGCTACAACGA GGAACTTT | 7232 |
| 3075 | UUCCGUGA G UUGAUCAU | 2503 | ATGATCAA GGCTAGCTACAACGA TCACGGAA | 7233 |
| 3079 | GUGAGUUG G UCAUCGAA | 2922 | TTCGATGA GGCTAGCTACAACGA CAACTCAC | 7234 |
| 3082 | AGUUGAUC G UCGAAUUC | 1526 | GAATTCGA GGCTAGCTACAACGA GATCAACT | 7235 |
| 3087 | AUCAUCGA G UUCUCCAA | 2923 | TTGGAGAA GGCTAGCTACAACGA TCGATGAT | 7236 |
| 3097 | UCUCCAAA G UGGCCCGA | 2924 | TCGGGCCA GGCTAGCTACAACGA TTTGGAGA | 7237 |
| 3100 | CCAAAAUG G CCCGAGAC | 2504 | GTCTCGGG GGCTAGCTACAACGA CATTTTGG | 7238 |
| 3107 | GGCCCGAG G CCCCCAGC | 2925 | GCTGGGGG GGCTAGCTACAACGA CTCGGGCC | 7239 |
| 3114 | GACCCCCA G CGCUACCU | 2505 | AGGTAGCG GGCTAGCTACAACGA TGGGGGTC | 7240 |
| 3116 | CCCCCAGC G CUACCUUG | 2506 | CAAGGTAG GGCTAGCTACAACGA GCTGGGGG | 7241 |
| 3119 | CCAGCGCU G CCUUGUCA | 360 | TGACAAGG GGCTAGCTACAACGA AGCGCTGG | 7242 |
| 3124 | GCUACCUU G UCAUUCAG | 2507 | CTGAATGA GGCTAGCTACAACGA AAGGTAGC | 7243 |
| 3127 | ACCUUGUC G UUCAGGGG | 1540 | CCCCTGAA GGCTAGCTACAACGA GACAAGGT | 7244 |
| 3137 | UCAGGGGG G UGAAAGAA | 2926 | TTCTTTCA GGCTAGCTACAACGA CCCCCTGA | 7245 |
| 3145 | AUGAAAGA G UGCAUUUG | 2927 | CAAATGCA GGCTAGCTACAACGA TCTTTCAT | 7246 |
| 3147 | GAAAGAAU G CAUUUGCC | 2508 | GGCAAATG GGCTAGCTACAACGA ATTCTTTC | 7247 |
| 3149 | AAGAAUGC G UUUGCCAA | 1542 | TTGGCAAA GGCTAGCTACAACGA GCATTCTT | 7248 |
| 3153 | AUGCAUUU G CCAAGUCC | 2509 | GGACTTGG GGCTAGCTACAACGA AAATGCAT | 7249 |
| 3158 | UUUGCCAA G UCCUACAG | 2510 | CTGTAGGA GGCTAGCTACAACGA TTGGCAAA | 7250 |
| 3163 | CAAGUCCU G CAGACUCC | 368 | GGAGTCTG GGCTAGCTACAACGA AGGACTTG | 7251 |
| 3167 | UCCUACAG G CUCCAACU | 2928 | AGTTGGAG GGCTAGCTACAACGA CTGTAGGA | 7252 |
| 3173 | AGACUCCA G CUUCUACC | 2929 | GGTAGAAG GGCTAGCTACAACGA TGGAGTCT | 7253 |
| 3179 | CAACUUCU G CCGUGCCC | 372 | GGGCACGG GGCTAGCTACAACGA AGAAGTTG | 7254 |
| 3182 | CUUCUACC G UGCCCUGA | 2511 | TCAGGGCA GGCTAGCTACAACGA GGTAGAAG | 7255 |
| 3184 | UCUACCGU G CCCUGAUG | 2512 | CATCAGGG GGCTAGCTACAACGA ACGGTAGA | 7256 |
| 3190 | GUGCCCUG G UGGAUGAA | 2930 | TTCATCCA GGCTAGCTACAACGA CAGGGCAC | 7257 |
| 3194 | CCUGAUGG G UGAAGAAG | 2931 | CTTCTTCA GGCTAGCTACAACGA CCATCAGG | 7258 |
| 3203 | UGAAGAAG G CAUGGACG | 2932 | CGTCCATG GGCTAGCTACAACGA CTTCTTCA | 7259 |
| 3205 | AAGAAGAC G UGGACGAC | 1557 | GTCGTCCA GGCTAGCTACAACGA GTCTTCTT | 7260 |
| 3209 | AGACAUGG G CGACGUGG | 2933 | CCACGTCG GGCTAGCTACAACGA CCATGTCT | 7261 |
| 3212 | CAUGGACG G CGUGGUGG | 2934 | CCACCACG GGCTAGCTACAACGA CGTCCATG | 7262 |
| 3214 | UGGACGAC G UGGUGGAU | 2513 | ATCCACCA GGCTAGCTACAACGA GTCGTCCA | 7263 |
| 3217 | ACGACGUG G UGGAUGCC | 2514 | GGCATCCA GGCTAGCTACAACGA CACGTCGT | 7264 |
| 3221 | CGUGGUGG G UGCCGACG | 2935 | CGTCGGCA GGCTAGCTACAACGA CCACCACG | 7265 |
| 3223 | UGGUGGAU G CCGACGAG | 2515 | CTCGTCGG GGCTAGCTACAACGA ATCCACCA | 7266 |
| 3227 | GGAUGCCG G CGAGUACC | 2936 | GGTACTCG GGCTAGCTACAACGA CGGCATCC | 7267 |
| 3231 | GCCGACGA G UACCUCAU | 2516 | ATGAGGTA GGCTAGCTACAACGA TCGTCGGC | 7268 |
| 3233 | CGACGAGU G CCUCAUCC | 373 | GGATGAGG GGCTAGCTACAACGA ACTCGTCG | 7269 |
| 3238 | AGUACCUC G UCCCACAG | 1561 | CTGTGGGA GGCTAGCTACAACGA GAGGTACT | 7270 |
| 3243 | CUCAUCCC G CAGCAGGG | 1564 | CCCTGCTG GGCTAGCTACAACGA GGGATGAG | 7271 |
| 3246 | AUCCCACA G CAGGGCUU | 2517 | AAGCCCTG GGCTAGCTACAACGA TGTGGGAT | 7272 |
| 3251 | ACAGCAGG G CUUCUUCA | 2518 | TGAAGAAG GGCTAGCTACAACGA CCTGCTGT | 7273 |
| 3260 | CUUCUUCA G CAGCCCCU | 2519 | AGGGGCTG GGCTAGCTACAACGA TGAAGAAG | 7274 |
| 3263 | CUUCAGCA G CCCCUCCA | 2520 | TGGAGGCG GGCTAGCTACAACGA TGCTGAAG | 7275 |
| 3271 | GCCCCUCC G CGUCACGG | 1576 | CCGTGACG GGCTAGCTACAACGA CGAGGGGC | 7276 |
| 3273 | CCCUCCAC G UCACGGAC | 2521 | GTCCGTGA GGCTAGCTACAACGA GTGGAGGG | 7277 |
| 3276 | UCCACGUC G CGGACUCC | 1577 | GGAGTCCG GGCTAGCTACAACGA GACGTGGA | 7278 |
| 3280 | CGUCACGG G CUCCCCUC | 2937 | GAGGGGAG GGCTAGCTACAACGA CCGTGACG | 7279 |
| 3293 | CCUCCUGA G CUCUCUGA | 2522 | TCAGAGAG GGCTAGCTACAACGA TCAGGAGG | 7280 |
| 3302 | CUCUCUGA G UGCAACCA | 2523 | TGGTTGCA GGCTAGCTACAACGA TCAGAGAG | 7281 |
| 3304 | CUCUGAGU G CAACCAGC | 2524 | GCTGGTTG GGCTAGCTACAACGA ACTCAGAG | 7282 |
| 3307 | UGAGUGCA G CCAGCAAC | 2938 | GTTGCTGG GGCTAGCTACAACGA TGCACTCA | 7283 |
| 3311 | UGCAACCA G CAACAAUU | 2525 | AATTGTTG GGCTAGCTACAACGA TGGTTGCA | 7284 |
| 3314 | AACCAGCA G CAAUUCCA | 2939 | TGGAATTG GGCTAGCTACAACGA TGCTGGTT | 7285 |
| 3317 | CAGCAACA G UUCCACCG | 2940 | CGGTGGAA GGCTAGCTACAACGA TGTTGCTG | 7286 |
| 3322 | ACAAUUCC G CCGUGGCU | 1594 | AGCCACGG GGCTAGCTACAACGA GGAATTGT | 7287 |
| 3325 | AUUCCACC G UGGCUUGC | 2526 | GCAAGCCA GGCTAGCTACAACGA GGTGGAAT | 7288 |
| 3328 | CCACCGUG G CUUGCAUU | 2527 | AATGCAAG GGCTAGCTACAACGA CACGGTGG | 7289 |
| 3332 | CGUGGCUU G CAUUGAUA | 2528 | TATCAATG GGCTAGCTACAACGA AAGCCACG | 7290 |
| 3334 | UGGCUUGC G UUGAUAGA | 1597 | TCTATCAA GGCTAGCTACAACGA GCAAGCCA | 7291 |
| 3338 | UUGCAUUG G UAGAAAUG | 2941 | CATTTCTA GGCTAGCTACAACGA CAATGCAA | 7292 |
| 3344 | UGAUAGAA G UGGGCUGC | 2942 | GCAGCCCA GGCTAGCTACAACGA TTCTATCA | 7293 |
| 3348 | AGAAAUGG G CUGCAAAG | 2529 | CTTTGCAG GGCTAGCTACAACGA CCATTTCT | 7294 |
| 3351 | AAUGGGCU G CAAAGCUG | 2530 | CAGCTTTG GCCTAGCTACAACGA AGCCCATT | 7295 |
| 3356 | GCUGCAAA G CUGUCCCA | 2531 | TGGGACAG GGCTAGCTACAACGA TTTGCAGC | 7296 |
| 3359 | GCAAACCU G UCCCAUCA | 2532 | TGATGGGA GGCTAGCTACAACGA AGCTTTGC | 7297 |
| 3364 | GCUGUCCC G UCAAGGAA | 1603 | TTCCTTGA GGCTAGCTACAACGA GGGACAGC | 7298 |
| 3374 | CAAGGAAG G CAGCUUCU | 2943 | AGAAGCTG GGCTAGCTACAACGA CTTCCTTG | 7299 |
| 3377 | GGAAGACA G CUUCUUGC | 2533 | GCAAGAAG GGCTAGCTACAACGA TGTCTTCC | 7300 |
| 3384 | AGCUUCUU G CAGCGAUA | 2534 | TATCGCTG GCCTAGCTACAACGA AAGAACCT | 7301 |
| 3387 | UUCUUCCA G CGAUACAG | 2535 | CTGTATCC GGCTAGCTACAACGA TGCAAGAA | 7302 |
| 3390 | UUGCAGCG G UACAGCUC | 2944 | GACCTGTA GGCTAGCTACAACGA CGCTGCAA | 7303 |
| 3392 | GCAGCCAU G CAGCUCAC | 396 | CTCACCTG GCCTACCTACAACGA ATCGCTCC | 7304 |
| 3395 | GCGAUACA G CUCAGACC | 2536 | GCTCTCAC GGCTAGCTACAACCA TCTATCCC | 7305 |
| 3401 | CAGCUCAC G CCCCACAC | 2945 | CTGTGGCG GCCTAGCTACAACGA CTCACCTC | 7306 |
| 3406 | CACACCCC G CACGCGCC | 1615 | CGCCCCTG GCCTAGCTACAACGA CCCGTCTC | 7307 |
| 3410 | CCCCACAC G CGCCUUGA | 2537 | TCAAGCCC GCCTACCTACAACGA CTCTCCCC | 7308 |
| 3412 | CCACAGGC G CCUUCACU | 2538 | AGTCAAGG GGCTAGCTACAACGA GCCTGTCG | 7309 |
| 3418 | CCCCCUUG G CUCACCAC | 2946 | CTCCTCAG GGCTACCTACAACCA CAACGCGC | 7310 |
| 3425 | GACUGACG G CAGCAUAG | 2947 | CTATGCTC GGCTAGCTACAACGA CCTCAGTC | 7311 |
| 3428 | UCAGGACA G CAUAGACG | 2539 | CCTCTATC GGCTAGCTACAACGA TGTCCTCA | 7312 |
| 3430 | AGCACACC G UAGACCAC | 1621 | GTCGTCTA GGCTAGCTACAACGA GCTGTCCT | 7313 |
| 3434 | CACCAUAG G CGACACCU | 2948 | ACCTGTCC GCCTAGCTACAACGA CTATGCTC | 7314 |
| 3437 | CAUACACC G CACCUUCC | 2949 | CCAAGGTG GCCTAGCTACAACGA CGTCTATG | 7315 |
| 3439 | UAGACGAC G CCUUCCUC | 1622 | GACCAACC GCCTAGCTACAACGA GTCGTCTA | 7316 |
| 3451 | UCCUCCCA G UCCCUCAA | 2540 | TTCAGGCA GGCTAGCTACAACGA TCCGAGGA | 7317 |
| 3453 | CUCCCACU G CCUGAAUA | 2541 | TATTCAGG GGCTAGCTACAACGA ACTCGGAC | 7318 |
| 3459 | GUCCCUCA G UACAUAAA | 2950 | TTTATGTA GGCTAGCTACAACGA TCACGCAC | 7319 |
| 3461 | GCCUCAAU G CAUAAACC | 403 | GGTTTATC GCCTACCTACAACGA ATTCAGGC | 7320 |
| 3463 | CUCAAUAC G UAAACCAG | 1632 | CTGGTTTA GCCTAGCTACAACGA GTATTCAG | 7321 |
| 3467 | AUACAUAA G CCACUCCC | 2951 | CCGACTCC GCCTAGCTACAACGA TTATGTAT | 7322 |
| 3471 | AUAAACCA G UCCCUUCC | 2542 | CCAACCCA GGCTAGCTACAACGA TCCTTTAT | 7323 |
| 3475 | ACCACUCC G UUCCCAAA | 2543 | TTTCCCAA GCCTACCTACAACGA CCACTCCT | 7324 |
| 3486 | CCCAAAAC G CCCCCUCC | 2544 | CCACCCGG GCCTAGCTACAACGA CTTTTCCG | 7325 |
| 3490 | AAAGCCCC G CUCCCUCU | 2545 | ACACCCAC GCCTACCTACAACGA CCCCCTTT | 7326 |
| 3494 | GCCCGCUG G CUCUCUCC | 2546 | GCACACAG GGCTAGCTACAACGA CAGCGGGC | 7327 |
| 3499 | CUCCCUCU G UGCACAAU | 2547 | ATTCTCCA GCCTACCTACAACGA ACACCCAC | 7328 |
| 3501 | CCCUCUGU G CACAAUCC | 2548 | CGATTCTC GCCTACCTACAACGA ACACACCC | 7329 |
| 3506 | UCUCCAGA G UCCUCUCU | 2952 | AGACACCA GCCTACCTACAACGA TCTCCACA | 7330 |
| 3511 | ACAAUCCU G UCUAUCAC | 2549 | CTCATACA GCCTACCTACAAGCA ACCATTCT | 7331 |
| 3515 | UCCUGUCU G UCACAAUC | 411 | GATTGTGA GGCTAGCTACAACGA AGACAGGA | 7332 |
| 3518 | UGUCUAUC G CAAUCAGC | 1648 | GCTGATTG GGCTAGCTACAACGA GATAGACA | 7333 |
| 3521 | CUAUCACA G UCAGCCUC | 2953 | GAGGCTGA GGCTAGCTACAACGA TGTGATAG | 7334 |
| 3525 | CACAAUCA G CCUCUGAA | 2550 | TTCAGAGG GGCTAGCTACAACGA TGATTGTG | 7335 |
| 3533 | GCCUCUGA G CCCCGCGC | 2954 | GCGCGGGG GGCTAGCTACAACGA TCAGAGGC | 7336 |
| 3538 | UGAACCCC G CGCCCAGC | 2551 | GCTGGGCG GGCTAGCTACAACGA GGGGTTCA | 7337 |
| 3540 | AACCCCGC G CCCAGCAG | 2552 | CTGCTGGG GGCTAGCTACAACGA GCGGGGTT | 7338 |
| 3545 | CGCGCCCA G CAGAGACC | 2553 | GGTCTCTG GGCTAGCTACAACGA TGGGCGCG | 7339 |
| 3551 | CAGCAGAG G CCCACACU | 2955 | AGTGTGGG GGCTAGCTACAACGA CTCTGCTG | 7340 |
| 3555 | AGAGACCC G CACUACCA | 1663 | TGGTAGTG GGCTAGCTACAACGA GGGTCTCT | 7341 |
| 3557 | AGACCCAC G CUACCAGG | 1664 | CCTGGTAG GGCTAGCTACAACGA GTGGGTCT | 7342 |
| 3560 | CCCACACU G CCAGGACC | 415 | GGTCCTGG GGCTAGCTACAACGA AGTGTGGG | 7343 |
| 3566 | CUACCAGG G CCCCCACA | 2956 | TGTGGGGG GGCTAGCTACAACGA CCTGGTAG | 7344 |
| 3572 | GGACCCCC G CAGCACUG | 1672 | CAGTGCTG GGCTAGCTACAACGA GGGGGTCC | 7345 |
| 3575 | CCCCCACA G CACUGCAG | 2554 | CTGCAGTG GGCTAGCTACAACGA TGTGGGGG | 7346 |
| 3577 | CCCACAGC G CUGCAGUG | 1674 | CACTGCAG GGCTAGCTACAACGA GCTGTGGG | 7347 |
| 3580 | ACAGCACU G CAGUGGGC | 2555 | GCCCACTG GGCTAGCTACAACGA AGTGCTGT | 7348 |
| 3583 | GCACUGCA G UGGGCAAC | 2556 | GTTGCCCA GGCTAGCTACAACGA TGCAGTGC | 7349 |
| 3587 | UGCAGUGG G CAACCCCG | 2557 | CGGGGTTG GGCTAGCTACAACGA CCACTGCA | 7350 |
| 3590 | AGUGGGCA G CCCCGAGU | 2957 | ACTCGGGG GGCTAGCTACAACGA TGCCCACT | 7351 |
| 3597 | AACCCCGA G UAUCUCAA | 2558 | TTGAGATA GGCTAGCTACAACGA TCGGGGTT | 7352 |
| 3599 | CCCCGAGU G UCUCAACA | 416 | TGTTGAGA GGCTAGCTACAACGA ACTCGGGG | 7353 |
| 3605 | GUAUCUCA G CACUGUCC | 2958 | GGACAGTG GGCTAGCTACAACGA TGAGATAC | 7354 |
| 3607 | AUCUCAAC G CUGUCCAG | 1683 | CTGGACAG GGCTAGCTACAACGA GTTGAGAT | 7355 |
| 3610 | UCAACACU G UCCAGCCC | 2559 | GGGCTGGA GGCTAGCTACAACGA AGTGTTGA | 7356 |
| 3615 | ACUGUCCA G CCCACCUG | 2560 | CAGGTGGG GGCTAGCTACAACGA TGGACAGT | 7357 |
| 3619 | UCCAGCCC G CCUGUGUC | 1689 | GACACAGG GGCTAGCTACAACGA GGGCTGGA | 7358 |
| 3623 | GCCCACCU G UGUCAACA | 2561 | TGTTGACA GGCTAGCTACAACGA AGGTGGGC | 7359 |
| 3625 | CCACCUGU G UCAACAGC | 2562 | GCTGTTGA GGCTAGCTACAACGA ACAGGTGG | 7360 |
| 3629 | CUGUGUCA G CAGCACAU | 2959 | ATGTGCTG GGCTAGCTACAACGA TGACACAG | 7361 |
| 3632 | UGUCAACA G CACAUUCG | 2563 | CGAATGTG GGCTAGCTACAACGA TGTTGACA | 7362 |
| 3634 | UCAACAGC G CAUUCGAC | 1694 | GTCGAATG GGCTAGCTACAACGA GCTGTTGA | 7363 |
| 3636 | AACAGCAC G UUCGACAG | 1695 | CTGTCGAA GGCTAGCTACAACGA GTGCTGTT | 7364 |
| 3641 | CACAUUCG G CAGCCCUG | 2960 | CAGGGCTG GGCTAGCTACAACGA CGAATGTG | 7365 |
| 3644 | AUUCGACA G CCCUGCCC | 2564 | GGGCAGGG GGCTAGCTACAACGA TGTCGAAT | 7366 |
| 3649 | ACAGCCCU G CCCACUGG | 2565 | CCAGTGGG GGCTAGCTACAACGA AGGGCTGT | 7367 |
| 3653 | CCCUGCCC G CUGGGCCC | 1702 | GGGCCCAG GGCTAGCTACAACGA GGGCAGGG | 7368 |
| 3658 | CCCACUGG G CCCAGAAA | 2566 | TTTCTGGG GGCTAGCTACAACGA CCAGTGGG | 7369 |
| 3668 | CCAGAAAG G CAGCCACC | 2567 | GGTGGCTG GGCTAGCTACAACGA CTTTCTGG | 7370 |
| 3671 | GAAAGGCA G CCACCAAA | 2568 | TTTGGTGG GGCTAGCTACAACGA TGCCTTTC | 7371 |
| 3674 | AGGCAGCC G CCAAAUUA | 1709 | TAATTTGG GGCTAGCTACAACGA GGCTGCCT | 7372 |
| 3679 | GCCACCAA G UUAGCCUG | 2961 | CAGGCTAA GGCTAGCTACAACGA TTGGTGGC | 7373 |
| 3683 | CCAAAUUA G CCUGGACA | 2569 | TGTCCAGG GGCTAGCTACAACGA TAATTTGG | 7374 |
| 3689 | UAGCCUGG G CAACCCUG | 2962 | CAGGGTTG GGCTAGCTACAACGA CCAGGCTA | 7375 |
| 3692 | CCUGGACA G CCCUGACU | 2963 | AGTCAGGG GGCTAGCTACAACGA TGTCCAGG | 7376 |
| 3698 | CAACCCUG G CUACCAGC | 2964 | GCTGGTAG GGCTAGCTACAACGA CAGGGTTG | 7377 |
| 3701 | CCCUGACU G CCAGCAGG | 425 | CCTGCTGG GGCTAGCTACAACGA AGTCAGGG | 7378 |
| 3705 | GACUACCA G CAGGACUU | 2570 | AAGTCCTG GGCTAGCTACAACGA TGGTAGTC | 7379 |
| 3710 | CCAGCAGG G CUUCUUUC | 2965 | GAAAGAAG GGCTAGCTACAACGA CCTGCTGG | 7380 |
| 3727 | CCAAGGAA G CCAAGCCA | 2571 | TGGCTTGG GGCTAGCTACAACGA TTCCTTGG | 7381 |
| 3732 | GAAGCCAA G CCAAAUGG | 2572 | CCATTTGG GGCTAGCTACAACGA TTGGCTTC | 7382 |
| 3737 | CAAGCCAA G UGGCAUCU | 2966 | AGATGCCA GGCTAGCTACAACGA TTGGCTTG | 7383 |
| 3740 | GCCAAAUG G CAUCUUUA | 2573 | TAAAGATG GGCTAGCTACAACGA CATTTGGC | 7384 |
| 3742 | CAAAUGGC G UCUUUAAG | 1731 | CTTAAAGA GGCTAGCTACAACGA GCCATTTG | 7385 |
| 3752 | CUUUAAGG G CUCCACAG | 2574 | CTGTGGAG GGCTAGCTACAACGA CCTTAAAG | 7386 |
| 3757 | AGGGCUCC G CAGCUGAA | 1735 | TTCAGCTG GGCTAGCTACAACGA GGAGCCCT | 7387 |
| 3760 | GCUCCACA G CUGAAAAU | 2575 | ATTTTCAG GGCTAGCTACAACGA TGTGGAGC | 7388 |
| 3767 | AGCUGAAA G UGCAGAAU | 2967 | ATTCTGCA GGCTAGCTACAACGA TTTCAGCT | 7389 |
| 3769 | CUGAAAAU G CAGAAUAC | 2576 | GTATTCTG GGCTAGCTACAACGA ATTTTCAG | 7390 |
| 3774 | AAUGCAGA G UACCUAAG | 2968 | CTTAGGTA GGCTAGCTACAACGA TCTGCATT | 7391 |
| 3776 | UGCAGAAU G CCUAAGGG | 436 | CCCTTAGG GGCTAGCTACAACGA ATTCTGCA | 7392 |
| 3784 | ACCUAAGG G UCGCGCCA | 2577 | TGGCGCGA GGCTAGCTACAACGA CCTTAGGT | 7393 |
| 3787 | UAAGGGUC G CGCCACAA | 2578 | TTGTGGCG GGCTAGCTACAACGA GACCCTTA | 7394 |
| 3789 | AGGGUCGC G CCACAAAG | 2579 | CTTTGTGG GGCTAGCTACAACGA GCGACCCT | 7395 |
| 3792 | GUCGCGCC G CAAAGCAG | 1742 | CTGCTTTG GGCTAGCTACAACGA GGCGCGAC | 7396 |
| 3797 | GCCACAAA G CAGUGAAU | 2580 | ATTCACTG GGCTAGCTACAACGA TTTGTGGC | 7397 |
| 3800 | ACAAAGCA G UGAAUUUA | 2581 | TAAATTCA GGCTAGCTACAACGA TGCTTTGT | 7398 |
| 3804 | AGCAGUGA G UUUAUUGG | 2969 | CCAATAAA GGCTAGCTACAACGA TCACTGCT | 7399 |
| 3808 | GUGAAUUU G UUGGAGCA | 441 | TGCTCCAA GGCTAGCTACAACGA AAATTCAC | 7400 |
| 3814 | UUAUUGGA G CAUGACCA | 2582 | TGGTCATG GGCTAGCTACAACGA TCCAATAA | 7401 |
| 3816 | AUUGGAGC G UGACCACG | 1745 | CGTGGTCA GGCTAGCTACAACGA GCTCCAAT | 7402 |
| 3819 | GGAGCAUG G CCACGGAG | 2970 | CTCCGTGG GGCTAGCTACAACGA CATGCTCC | 7403 |
| 3822 | GCAUGACC G CGGAGGAU | 1747 | ATCCTCCG GGCTAGCTACAACGA GGTCATGC | 7404 |
| 3829 | CACGGAGG G UAGUAUGA | 2971 | TCATACTA GGCTAGCTACAACGA CCTCCGTG | 7405 |
| 3832 | GGAGGAUA G UAUGAGCC | 2583 | GGCTCATA GGCTAGCTACAACGA TATCCTCC | 7406 |
| 3834 | AGGAUAGU G UGAGCCCU | 444 | AGGGCTCA GGCTAGCTACAACGA ACTATCCT | 7407 |
| 3838 | UAGUAUGA G CCCUAAAA | 2584 | TTTTAGGG GGCTAGCTACAACGA TCATACTA | 7408 |
| 3847 | CCCUAAAA G UCCAGACU | 2972 | AGTCTGGA GGCTAGCTACAACGA TTTTAGGU | 7409 |
| 3853 | AAAUCCAG G CUCUUUCG | 2973 | CGAAAGAG GGCTAGCTACAACGA CTGGATTT | 7410 |
| 3862 | CUCUUUCG G UACCCAGG | 2974 | CCTGGGTA GGCTAGCTACAACGA CGAAAGAG | 7411 |
| 3864 | CUUUCGAU G CCCAGGAC | 451 | GTCCTGGG GGCTAGCTACAACGA ATCGAAAG | 7412 |
| 3871 | UACCCAGG G CCAAGCCA | 2975 | TGGCTTGG GGCTAGCTACAACGA CCTGGGTA | 7413 |
| 3876 | AGGACCAA G CCACAGCA | 2585 | TGCTGTGG GGCTAGCTACAACGA TTGGTCCT | 7414 |
| 3879 | ACCAAGCC G CAGCAGGU | 1761 | ACCTGCTG GGCTAGCTACAACGA GGCTTGGT | 7415 |
| 3882 | AAGCCACA G CAGGUCCU | 2586 | AGGACCTG GGCTAGCTACAACGA TGTGGCTT | 7416 |
| 3886 | CACAGCAG G UCCUCCAU | 2587 | ATGGAGGA GGCTAGCTACAACGA CTGCTGTG | 7417 |
| 3893 | GGUCCUCC G UCCCAACA | 1767 | TGTTGGGA GGCTAGCTACAACGA GGAGGACC | 7418 |
| 3899 | CCAUCCCA G CAGCCAUG | 2976 | CATGGCTG GGCTAGCTACAACGA TGGGATGG | 7419 |
| 3902 | UCCCAACA G CCAUGCCC | 2588 | GGGCATGG GGCTAGCTACAACGA TGTTGGGA | 7420 |
| 3905 | CAACAGCC G UGCCCGCA | 1773 | TGCGGGCA GGCTAGCTACAACGA GGCTGTTG | 7421 |
| 3907 | ACAGCCAU G CCCGCAUU | 2589 AATGCGGG GGCTAGCTACAACGA ATGGCTGT | 7422 | |
| 3911 | CCAUGCCC G CAUUAGCU | 2590 | AGCTAATG GGCTAGCTACAACGA GGGCATGG | 7423 |
| 3913 | AUGCCCGC G UUAGCUCU | 1776 | AGAGCTAA GGCTAGCTACAACGA GCGGGCAT | 7424 |
| 3917 | CCGCAUUA G CUCUUAGA | 2591 TCTAAGAG GGCTAGCTACAACGA TAATGCGG | 7425 | |
| 3925 | GCUCUUAG G CCCACAGA | 2977 | TCTGTGGG GGCTAGCTACAACGA CTAAGAGC | 7426 |
| 3929 | UUAGACCC G CAGACUGG | 1781 | CCAGTCTG GGCTAGCTACAACGA GGGTCTAA | 7427 |
| 3933 | ACCCACAG G CUGGUUUU | 2978 | AAAACCAG GGCTAGCTACAACGA CTGTGGGT | 7428 |
| 3937 | ACAGACUG G UUUUGCAA | 2592 | TTGCAAAA GGCTAGCTACAACGA CAGTCTGT | 7429 |
| 3942 | CUGGUUUU G CAACGUUU | 2593 | AAACGTTG GGCTAGCTACAACGA AAAACCAG | 7430 |
| 3945 | GUUUUGCA G CGUUUACA | 2979 | TGTAAACG GGCTAGCTACAACGA TGCAAAAC | 7431 |
| 3947 | UUUGCAAC G UUUACACC | 2594 | GGTGTAAA GGCTAGCTACAACGA GTTGCAAA | 7432 |
| 3951 | CAACGUUU G CACCGACU | 465 | AGTCGGTG GGCTAGCTACAACGA AAACGTTG | 7433 |
| 3953 | ACGUUUAC G CCGACUAG | 1785 | CTAGTCGG GGCTAGCTACAACGA GTAAACGT | 7434 |
| 3957 | UUACACCG G CUAGCCAG | 2980 | CTGGCTAG GGCTAGCTACAACGA CGGTGTAA | 7435 |
| 3961 | ACCGACUA G CCAGGAAG | 2595 | CTTCCTGG GGCTAGCTACAACGA TAGTCGGT | 7436 |
| 3969 | GCCAGGAA G UACUUCCA | 2596 | TGGAAGTA GGCTAGCTACAACGA TTCCTGGC | 7437 |
| 3971 | CAGGAAGU G CUUCCACC | 467 | GGTGGAAG GGCTAGCTACAACGA ACTTCCTG | 7438 |
| 3977 | GUACUUCC G CCUCGGGC | 1792 | GCCCGAGG GGCTAGCTACAACGA GGAAGTAC | 7439 |
| 3984 | CACCUCGG G CACAUUUU | 2597 | AAAATGTG GGCTAGCTACAACGA CCGAGGTG | 7440 |
| 3986 | CCUCGGGC G CAUUUUGG | 1795 | CCAAAATG GGCTAGCTACAACGA GCCCGAGG | 7441 |
| 3988 | UCGGGCAC G UUUUGGGA | 1796 | TCCCAAAA GGCTAGCTACAACGA GTGCCCGA | 7442 |
| 3998 | UUUGGGAA G UEUCAUUC | 2598 | GAATGCAA GGCTAGCTACAACGA TTCCCAAA | 7443 |
| 4001 | GGGAAGUU G CAUUCCUU | 2599 | AAGGAATG GGCTAGCTACAACGA AACTTCCC | 7444 |
| 4003 | GAAGUUGC G UUCCUUUG | 1797 | CAAAGGAA GGCTAGCTACAACGA GCAACTTC | 7445 |
| 4011 | AUUCCUUU G UCUUCAAA | 2600 | TTTGAAGA GGCTAGCTACAACGA AAAGGAAT | 7446 |
| 4019 | GUCUUCAA G CUGUGAAG | 2981 | CTTCACAG GGCTAGCTACAACGA TTGAAGAC | 7447 |
| 4022 | UUCAAACU G UGAAGCAU | 2601 | ATGCTTCA GGCTAGCTACAACGA AGTTTGAA | 7448 |
| 4027 | ACUGUGAA G CAUUUACA | 2602 | TGTAAATG GGCTAGCTACAACGA TTCACAGT | 7449 |
| 4029 | UGUGAAGC G UUUACAGA | 1803 | TCTGTAAA GGCTAGCTACAACGA GCTTCACA | 7450 |
| 4033 | AAGCAUUU G CAGAAACG | 484 | CGTTTCTG GGCTAGCTACAACGA AAATGCTT | 7451 |
| 4039 | UUACAGAA G CGCAUCCA | 2982 | TGGATGCG GGCTAGCTACAACGA TTCTGTAA | 7452 |
| 4041 | ACAGAAAC G CAUCCAGC | 2603 | GCTGGATG GGCTAGCTACAACGA GTTTCTGT | 7453 |
| 4043 | AGAAACGC G UCCAGCAA | 1805 | TTGCTGGA GGCTAGCTACAACGA GCGTTTCT | 7454 |
| 4048 | CGCAUCCA G CAAGAAUA | 2604 | TATTCTTG GGCTAGCTACAACGA TGGATGCG | 7455 |
| 4054 | CAGCAAGA G UAUUGUCC | 2983 | GGACAATA GGCTAGCTACAACGA TCTTGCTG | 7456 |
| 4056 | GCAAGAAU G UUGUCCCU | 486 | AGGGACAA GGCTAGCTACAACGA ATTCTTGC | 7457 |
| 4059 | AGAAUAUU G UCCCUUUG | 2605 | CAAAGGGA GGCTAGCTACAACGA AATATTCT | 7458 |
| 4069 | CCCUUUGA G CAGAAAUU | 2606 | AATTTCTG GGCTAGCTACAACGA TCAAAGGG | 7459 |
| 4075 | GAGCAGAA G UUUAUCUU | 2984 | AAGATAAA GGCTAGCTACAACGA TTCTGCTC | 7460 |
| 4079 | AGAAAUUU G UCUUUCAA | 493 | TTGAAAGA GGCTAGCTACAACGA AAATTTCT | 7461 |
| 4092 | UCAAAGAG G UAUAUUUG | 2607 | CAAATATA GGCTAGCTACAACGA CTCTTTGA | 7462 |
| 4094 | AAAGAGGU G UAUUUGAA | 498 | TTCAAATA GGCTAGCTACAACGA ACCTCTTT | 7463 |
| 4096 | AGAGGUAU G UUUGAAAA | 499 | TTTTCAAA GGCTAGCTACAACGA ATACCTCT | 7464 |
| 4116 | AAAAAAAA G UAUAUGUG | 2608 | CACATATA GGCTAGCTACAACGA TTTTTTTT | 7465 |
| 4118 | AAAAAAGU G UAUGUGAG | 502 | CTCACATA GGCTAGCTACAACGA ACTTTTTT | 7466 |
| 4120 | AAAAGUAU G UGUGAGGA | 503 | TCCTCACA GGCTAGCTACAACGA ATACTTTT | 7467 |
| 4122 | AAGUAUAU G UGAGGAUU | 2609 | AATCCTCA GGCTAGCTACAACGA ATATACTT | 7468 |
| 4128 | AUGUGAGG G UUUUUAUU | 2985 | AATAAAAA GGCTAGCTACAACGA CCTCACAT | 7469 |
| 4134 | GGAUUUUU G UUGAUUGG | 508 | CCAATCAA GGCTAGCTACAACGA AAAAATCC | 7470 |
| 4138 | UUUUAUUG G UUGGGGAU | 2986 | ATCCCCAA GGCTAGCTACAACGA CAATAAAA | 7471 |
| 4145 | GAUUGGGG G UCUUGGAG | 2987 | CTCCAAGA GGCTAGCTACAACGA CCCCAATC | 7472 |
| 4153 | AUCUUGGA G UUUUUCAU | 2610 | ATGAAAAA GGCTAGCTACAACGA TCCAAGAT | 7473 |
| 4160 | AGUUUUUC G UUGUCGCU | 1816 | AGCGACAA GGCTAGCTACAACGA GAAAAACT | 7474 |
| 4163 | UUUUCUUU G UCGCUAUU | 2611 | AATAGCGA GGCTAGCTACAACGA AATGAAAA | 7475 |
| 4166 | UCAUUGUC G CUAUUGAU | 2612 | ATCAATAG GGCTAGCTACAACGA GACAATGA | 7476 |
| 4169 | UUGUCGCU G UUGAUUUU | 520 | AAAATCAA GGCTAGCTACAACGA AGCGACAA | 7477 |
| 4173 | CGCUAUUG G UUUUUACU | 2988 | AGTAAAAA GGCTAGCTACAACGA CAATAGCG | 7478 |
| 4179 | UGAUUUUU G CUUCAAUG | 526 | CATTGAAG GGCTAGCTACAACGA AAAAATCA | 7479 |
| 4185 | UUACUUCA G UGGGCUCU | 2989 | AGAGCCCA GGCTAGCTACAACGA TGAAGTAA | 7480 |
| 4189 | UUCAAUGG G CUCUUCCA | 2613 | TGGAAGAG GGCTAGCTACAACGA CCATTGAA | 7481 |
| 4198 | CUCUUCCA G CAAGGAAG | 2990 | CTTCCTTG GGCTAGCTACAACGA TGGAAGAG | 7482 |
| 4209 | AGGAAGAA G CUUGCUGG | 2614 | CCAGCAAG GGCTAGCTACAACGA TTCTTCCT | 7483 |
| 4213 | AGAAGCUU G CUGGUAGC | 2615 | GCTACCAG GGCTAGCTACAACGA AAGCTTCT | 7484 |
| 4217 | GCUUGCUG G UAGCACUU | 2616 | AAGTGCTA GGCTAGCTACAACGA CAGCAAGC | 7485 |
| 4220 | UGCUGGUA G CACUUGCU | 2617 | AGCAAGTG GGCTAGCTACAACGA TACCAGCA | 7486 |
| 4222 | CUGGUAGC G CUUGCUAC | 1827 | GTAGCAAG GGCTAGCTACAACGA GCTACCAG | 7487 |
| 4226 | UAGCACUU G CUACCCUG | 2618 | CAGGGTAG GGCTAGCTACAACGA AAGTGCTA | 7488 |
| 4229 | CACUUGCU G CCCUGAGU | 535 | ACTCAGGG GGCTAGCTACAACGA AGCAAGTG | 7489 |
| 4236 | UACCCUGA G UUCAUCCA | 2619 | TGGATGAA GGCTAGCTACAACGA TCAGGGTA | 7490 |
| 4240 | CUGAGUUC G UCCAGGCC | 1833 | GGCCTGGA GGCTAGCTACAACGA GAACTCAG | 7491 |
| 4246 | UCAUCCAG G CCCAACUG | 2620 | CAGTTGGG GGCTAGCTACAACGA CTGGATGA | 7492 |
| 4251 | CAGGCCCA G CUGUGAGC | 2991 | GCTCACAG GGCTAGCTACAACGA TGGGCCTG | 7493 |
| 4254 | GCCCAACU G UGAGCAAG | 2621 | CTTGCTCA GGCTAGCTACAACGA AGTTGGGC | 7494 |
| 4258 | AACUGUGA G CAAGGAGC | 2622 | GCTCCTTG GGCTAGCTACAACGA TCACAGTT | 7495 |
| 4265 | AGCAAGGA G CACAAGCC | 2623 | GGCTTGTG GGCTAGCTACAACGA TCCTTGCT | 7496 |
| 4267 | CAAGGAGC G CAAGCCAC | 1841 | GTGGCTTG GGCTAGCTACAACGA GCTCCTTG | 7497 |
| 4271 | GAGCACAA G CCACAAGU | 2624 | ACTTGTGG GGCTAGCTACAACGA TTGTGCTC | 7498 |
| 4274 | CACAAGCC G CAAGUCUU | 1844 | AAGACTTG GGCTAGCTACAACGA GGCTTGTG | 7499 |
| 4278 | AGCCACAA G UCUUCCAG | 2625 | CTGGAAGA GGCTAGCTACAACGA TTGTGGCT | 7500 |
| 4290 | UCCAGAGG G UGCUUGAU | 2992 | ATCAAGCA GGCTAGCTACAACGA CCTCTGGA | 7501 |
| 4292 | CAGAGGAU G CUUGAUUC | 2626 | GAATCAAG GGCTAGCTACAACGA ATCCTCTG | 7502 |
| 4297 | GAUGCUUG G UUCCAGUG | 2993 | CACTGGAA GGCTAGCTACAACGA CAAGCATC | 7503 |
| 4303 | UGAUUCCA G UGGUUCUG | 2627 | CAGAACCA GGCTAGCTACAACGA TGGAATCA | 7504 |
| 4306 | UUCCAGUG G UUCUGCUU | 2628 | AAGCAGAA GGCTAGCTACAACGA CACTGGAA | 7505 |
| 4311 | GUGGUUCU G CUUCAAGG | 2629 | CCTTGAAG GGCTAGCTACAACGA AGAACCAC | 7506 |
| 4319 | GCUUCAAG G CUUCCACU | 2630 | AGTGGAAG GGCTAGCTACAACGA CTTGAAGC | 7507 |
| 4325 | AGGCUUCC G CUGCAAAA | 1857 | TTTTGCAG GGCTAGCTACAACGA GGAAGCCT | 7508 |
| 4328 | CUUCCACU G CAAAACAC | 2631 | GTGTTTTG GGCTAGCTACAACGA AGTGGAAG | 7509 |
| 4333 | ACUGCAAA G CACUAAAG | 2994 | CTTTAGTG GGCTAGCTACAACGA TTTGCAGT | 7510 |
| 4335 | UGCAAAAC G CUAAAGAU | 1860 | ATCTTTAG GGCTAGCTACAACGA GTTTTGCA | 7511 |
| 4342 | CACUAAAG G UCCAAGAA | 2995 | TTCTTGGA GGCTAGCTACAACGA CTTTAGTG | 7512 |
| 4352 | CCAAGAAG G CCUUCAUG | 2632 | CATGAAGG GGCTAGCTACAACGA CTTCTTGG | 7513 |
| 4358 | AGGCCUUC G UGGCCCCA | 1866 | TGGGGCCA GGCTAGCTACAACGA GAAGGCCT | 7514 |
| 4361 | CCUUCAUG G CCCCAGCA | 2633 | TGCTGGGG GGCTAGCTACAACGA CATGAAGG | 7515 |
| 4367 | UGGCCCCA G CAGGCCGG | 2634 | CCGGCCTG GGCTAGCTACAACGA TGGGGCCA | 7516 |
| 4371 | CCCAGCAG G CCGGAUCG | 2635 | CGATCCGG GGCTAGCTACAACGA CTGCTGGG | 7517 |
| 4376 | CAGGCCGG G UCGGUACU | 2996 | AGTACCGA GGCTAGCTACAACGA CCGGCCTG | 7518 |
| 4380 | CCGGAUCG G UACUGUAU | 2636 | ATACAGTA GGCTAGCTACAACGA CGATCCGG | 7519 |
| 4382 | GGAUCGGU G CUGUAUCA | 556 | TGATACAG GGCTAGCTACAACGA ACCGATCC | 7520 |
| 4385 | UCGGUACU G UAUCAAGU | 2637 | ACTTGATA GGCTAGCTACAACGA AGTACCGA | 7521 |
| 4387 | GGUACUGU G UCAAGUCA | 557 | TGACTTGA GGCTAGCTACAACGA ACAGTACC | 7522 |
| 4392 | UGUAUCAA G UCAUGGCA | 2638 | TGCCATGA GGCTAGCTACAACGA TTGATACA | 7523 |
| 4395 | AUCAAGUC G UGGCAGGU | 1875 | ACCTGCCA GGCTAGCTACAACGA GACTTGAT | 7524 |
| 4398 | AAGUCAUG G CAGGUACA | 2639 | TGTACCTG GGCTAGCTACAACGA CATGACTT | 7525 |
| 4402 | CAUGGCAG G UACAGUAG | 2640 | CTACTGTA GGCTAGCTACAACGA CTGCCATG | 7526 |
| 4404 | UGGCAGGU G CAGUAGGA | 560 | TCCTACTG GGCTAGCTACAACGA ACCTGCCA | 7527 |
| 4407 | CAGGUACA G UAGGAUAA | 2641 | TTATCCTA GGCTAGCTACAACGA TGTACCTG | 7528 |
| 4412 | ACAGUAGG G UAAGCCAC | 2997 | GTGGCTTA GGCTAGCTACAACGA CCTACTGT | 7529 |
| 4416 | UAGGAUAA G CCACUCUG | 2642 | CAGAGTGG GGCTAGCTACAACGA TTATCCTA | 7530 |
| 4419 | GAUAAGCC G CUCUGUCC | 1879 | GGACAGAG GGCTAGCTACAACGA GGCTTATC | 7531 |
| 4424 | GCCACUCU G UCCCUUCC | 2643 | GGAAGGGA GGCTAGCTACAACGA AGAGTGGC | 7532 |
| 4436 | CUUCCUGG G CAAAGAAG | 2644 | CTTCTTTG GGCTAGCTACAACGA CCAGGAAG | 7533 |
| 4447 | AAGAAGAA G CGGAGGGG | 2998 | CCCCTCCG GGCTAGCTACAACGA TTCTTCTT | 7534 |
| 4456 | CGGAGGGG G UGAAUUCU | 2999 | AGAATTCA GGCTAGCTACAACGA CCCCTCCG | 7535 |
| 4460 | GGGGAUGA G UUCUUCCU | 3000 | AGGAAGAA GGCTAGCTACAACGA TCATCCCC | 7536 |
| 4472 | UUCCUUAG G CUUACUUU | 3001 | AAAGTAAG GGCTAGCTACAACGA CTAAGGAA | 7537 |
| 4476 | UUAGACUU G CUUUUGUA | 574 | TACAAAAG GGCTAGCTACAACGA AAGTCTAA | 7538 |
| 4482 | UUACUUUU G UAAAAAUG | 2645 | CATTTTTA GGCTAGCTACAACGA AAAAGTAA | 7539 |
| 4488 | UUGUAAAA G UGUCCCCA | 3002 | TGGGGACA GGCTAGCTACAACGA TTTTACAA | 7540 |
| 4490 | GUAAAAAU G UCCCCACG | 2646 | CGTGGGGA GGCTAGCTACAACGA ATTTTTAC | 7541 |
| 4496 | AUGUCCCC G CGGUACUU | 1896 | AAGTACCG GGCTAGCTACAACGA GGGGACAT | 7542 |
| 4499 | UCCCCACG G UACUUACU | 2647 | AGTAAGTA GGCTAGCTACAACGA CGTGGGGA | 7543 |
| 4501 | CCCACGGU G CUUACUCC | 580 | GGAGTAAG GGCTAGCTACAACGA ACCGTGGG | 7544 |
| 4505 | CGGUACUU G CUCCCCAC | 582 | GTGGGGAG GGCTAGCTACAACGA AAGTACCG | 7545 |
| 4512 | UACUCCCC G CUGAUGGA | 1902 | TCCATCAG GGCTAGCTACAACGA GGGGAGTA | 7546 |
| 4516 | CCCCACUG G UGGACCAG | 3003 | CTGGTCCA GGCTAGCTACAACGA CAGTGGGG | 7547 |
| 4520 | ACUGAUGG G CCAGUGGU | 3004 | ACCACTGG GGCTAGCTACAACGA CCATCAGT | 7548 |
| 4524 | AUGGACCA G UGGUUUCC | 2648 | GGAAACCA GGCTAGCTACAACGA TGGTCCAT | 7549 |
| 4527 | GACCAGUG G UUUCCAGU | 2649 | ACTGGAAA GGCTAGCTACAACGA CACTGGTC | 7550 |
| 4534 | GGUUUCCA G UCAUGAGC | 2650 | GCTCATGA GGCTAGCTACAACGA TGGAAACC | 7551 |
| 4537 | UUCCAGUC G UGAGCGUU | 1908 | AACGCTCA GGCTAGCTACAACGA GACTGGAA | 7552 |
| 4541 | AGUCAUGA G CGUUAGAC | 2651 | GTCTAACG GGCTAGCTACAACGA TCATGACT | 7553 |
| 4543 | UCAUGAGC G UUAGACUG | 2652 | CAGTCTAA GGCTAGCTACAACGA GCTCATGA | 7554 |
| 4548 | AGCGUUAG G CUGACUUU | 3005 | CAAGTCAG GGCTAGCTACAACGA CTAACGCT | 7555 |
| 4552 | UUAGACUG G CUUGUUUG | 3006 | CAAACAAG GGCTAGCTACAACGA CAGTCTAA | 7556 |
| 4556 | ACUGACUU G UUUGUCUU | 2653 | AAGACAAA GGCTAGCTACAACGA AAGTCAGT | 7557 |
| 4560 | ACUUGUUU G UCUUCCAU | 2654 | ATGGAAGA GGCTAGCTACAACGA AAACAAGT | 7558 |
| 4567 | UGUCUUCC G UUCCAUUG | 1913 | CAATGGAA GGCTAGCTACAACGA GGAAGACA | 7559 |
| 4572 | UCCAUUCC G UUGUUUUG | 1915 | CAAAACAA GGCTAGCTACAACGA GGAATGGA | 7560 |
| 4575 | AUUCCAUU G UUUUGAAA | 2655 | TTTCAAAA GGCTAGCTACAACGA AATGGAAT | 7561 |
| 4583 | UTUUUGAA G CUCAGUAU | 3007 | ATACTGAG GGCTAGCTACAACGA TTCAAAAC | 7562 |
| 4588 | GAAACUCA G UAUGCCGC | 2656 | GCGGCATA GGCTAGCTACAACGA TGAGTTTC | 7563 |
| 4590 | AACUCAGU G UGCCGCCC | 603 | GGGCGGCA GGCTAGCTACAACGA ACTGAGTT | 7564 |
| 4592 | CUCAGUAU G CCGCCCCU | 2657 | AGGGGCGG GGCTAGCTACAACGA ATACTGAG | 7565 |
| 4595 | AGUAUGCC G CCCCUGUC | 2658 | GACAGGGG GGCTAGCTACAACGA GGCATACT | 7566 |
| 4601 | CCGCCCCU G UCUUGCUG | 2659 | CAGCAAGA GGCTAGCTACAACGA AGGGGCGG | 7567 |
| 4606 | CCUGUCUU G CUGUCAUG | 2660 | CATGACAG GGCTAGCTACAACGA AAGACAGG | 7568 |
| 4609 | GUCUUGCU G UCAUGAAA | 2661 | TTTCATGA GGCTAGCTACAACGA AGCAAGAC | 7569 |
| 4612 | UUGCUGUC G UGAAAUCA | 1925 | TGATTTCA GGCTAGCTACAACGA GACAGCAA | 7570 |
| 4617 | GUCAUGAA G UCAGCAAG | 3008 | CTTGCTGA GGCTAGCTACAACGA TTCATGAC | 7571 |
| 4621 | UGAAAUCA G CAAGAGAG | 2662 | CTCTCTTG GGCTAGCTACAACGA TGATTTCA | 7572 |
| 4631 | AAGAGAGG G UGACACAU | 3009 | ATGTGTCA GGCTAGCTACAACGA CCTCTCTT | 7573 |
| 4634 | AGAGGAUG G CACAUCAA | 3010 | TTGATGTG GGCTAGCTACAACGA CATCCTCT | 7574 |
| 4636 | AGGAUGAC G CAUCAAAU | 1928 | ATTTGATG GGCTAGCTACAACGA GTCATCCT | 7575 |
| 4638 | GAUGACAC G UCAAAUAA | 1929 | TTATTTGA GGCTAGCTACAACGA GTGTCATC | 7576 |
| 4643 | CACAUCAA G UAAUAACU | 3011 | AGTTATTA GGCTAGCTACAACGA TTGATGTG | 7577 |
| 4646 | AUCAAAUA G UAACUCGG | 3012 | CCGAGTTA GGCTAGCTACAACGA TATTTGAT | 7578 |
| 4649 | AAAUAAUA G CUCGGAUU | 3013 | AATCCGAG GGCTAGCTACAACGA TATTATTT | 7579 |
| 4655 | UAACUCGG G UUCCAGCC | 3014 | GGCTGGAA GGCTAGCTACAACGA CCGAGTTA | 7580 |
| 4661 | GGAUUCCA G CCCACAUU | 2663 | AATGTGGG GGCTAGCTACAACGA TGGAATCC | 7581 |
| 4665 | UCCAGCCC G CAUUGGAU | 1936 | ATCCAATG GGCTAGCTACAACGA GGGCTGGA | 7582 |
| 4667 | CAGCCCAC G UUGGAUUC | 1937 | GAATCCAA GGCTAGCTACAACGA GTGGGCTG | 7583 |
| 4672 | CACAUUGG G UUCAUCAG | 3015 | CTGATGAA GGCTAGCTACAACGA CCAATGTG | 7584 |
| 4676 | UUGGAUUC G UCAGCAUU | 1938 | AATGCTGA GGCTAGCTACAACGA GAATCCAA | 7585 |
| 4680 | AUUCAUCA G CAUUUGGA | 2664 | TCCAAATG GGCTAGCTACAACGA TGATGAAT | 7586 |
| 4682 | UCAUCAGC G UUUGGACC | 1940 | GGTCCAAA GGCTAGCTACAACGA GCTGATGA | 7587 |
| 4688 | GCAUUUGG G CCAAUAGC | 3016 | GCTATTGG GGCTAGCTACAACGA CCAAATGC | 7588 |
| 4692 | UUGGACCA G UAGCCCAC | 3017 | GTGGGCTA GGCTAGCTACAACGA TGGTCCAA | 7589 |
| 4695 | GACCAAUA G CCCACAGC | 2665 | GCTGTGGG GGCTAGCTACAACGA TATTGGTC | 7590 |
| 4699 | AAUAGCCC G CAGCUGAG | 1945 | CTCAGCTG GGCTAGCTACAACGA GGGCTATT | 7591 |
| 4702 | AGCCCACA G CUGAGAAU | 2666 | ATTCTCAG GGCTAGCTACAACGA TGTGGGCT | 7592 |
| 4709 | AGCUGAGA G UGUGGAAU | 3018 | ATTCCACA GGCTAGCTACAACGA TCTCAGCT | 7593 |
| 4711 | CUGAGAAU G UGGAAUAC | 2667 | GTATTCCA GGCTAGCTACAACGA ATTCTCAG | 7594 |
| 4716 | AAUGUGGA G UACCUAAG | 3019 | CTTAGGTA GGCTAGCTACAACGA TCCACATT | 7595 |
| 4718 | UGUGGAAU G CCUAAGGA | 621 | TCCTTAGG GGCTAGCTACAACGA ATTCCACA | 7596 |
| 4726 | ACCUAAGG G UAACACCG | 3020 | CGGTGTTA GGCTAGCTACAACGA CCTTAGGT | 7597 |
| 4729 | UAAGGAUA G CACCGCUU | 3021 | AAGCGGTG GGCTAGCTACAACGA TATCCTTA | 7598 |
| 4731 | AGGAUAAC G CCGCUUUU | 1950 | AAAAGCGG GGCTAGCTACAACGA GTTATCCT | 7599 |
| 4734 | AUAACACC G CUUUUGUU | 2668 | AACAAAAG GGCTAGCTACAACGA GGTGTTAT | 7600 |
| 4740 | CCGCUUUU G UUCUCGCA | 2669 | TGCGAGAA GGCTAGCTACAACGA AAAAGCGG | 7601 |
| 4746 | UUGUUCUC G CAAAAACG | 2670 | CGTTTTTG GGCTAGCTACAACGA GAGAACAA | 7602 |
| 4752 | UCGCAAAA G CGUAUCUC | 3022 | GAGATACG GGCTAGCTACAACGA TTTTGCGA | 7603 |
| 4754 | GCAAAAAC G UAUCUCCU | 2671 | AGGAGATA GGCTAGCTACAACGA GTTTTTGC | 7604 |
| 4756 | AAAAACGU G UCUCCUAA | 630 | TTAGGAGA GGCTAGCTACAACGA ACGTTTTT | 7605 |
| 4764 | AUCUCCUA G UUUGAGGC | 3023 | GCCTCAAA GGCTAGCTACAACGA TAGGAGAT | 7606 |
| 4771 | AAUUUGAG G CUCAGAUG | 2672 | CATCTGAG GGCTAGCTACAACGA CTCAAATT | 7607 |
| 4777 | AGGCUCAG G UGAAAUGC | 3024 | GCATTTCA GGCTAGCTACAACGA CTGAGCCT | 7608 |
| 4782 | CAGAUGAA G UGCAUCAG | 3025 | CTGATGCA GGCTAGCTACAACGA TTCATCTG | 7609 |
| 4784 | GAUGAAAU G CAUCAGGU | 2673 | ACCTGATG GGCTAGCTACAACGA ATTTCATC | 7610 |
| 4786 | UGAAAUGC G UCAGGUCC | 1960 | GGACCTGA GGCTAGCTACAACGA GCATTTCA | 7611 |
| 4791 | UGCAUCAG G UCCUUUGG | 2674 | CCAAAGGA GGCTAGCTACAACGA CTGATGCA | 7612 |
| 4801 | CCUUUGGG G CAUAGAUC | 2675 | GATCTATG GGCTAGCTACAACGA CCCAAAGG | 7613 |
| 4803 | UUUGGGGC G UAGAUCAG | 1964 | CTGATCTA GGCTAGCTACAACGA GCCCCAAA | 7614 |
| 4807 | GGGCAUAG G UCAGAAGA | 3026 | TCTTCTGA GGCTAGCTACAACGA CTATGCCC | 7615 |
| 4815 | AUCAGAAG G CUACAAAA | 3027 | TTTTGTAG GGCTAGCTACAACGA CTTCTGAT | 7616 |
| 4818 | AGAAGACU G CAAAAAUG | 643 | CATTTTTG GGCTAGCTACAACGA AGTCTTCT | 7617 |
| 4824 | CUACAAAA G UGAAGCUG | 3028 | CAGCTTCA GGCTAGCTACAACGA TTTTGTAG | 7618 |
| 4829 | AAAAUGAA G CUGCUCUG | 2676 | CAGACCAG GGCTAGCTACAACGA TTCATTTT | 7619 |
| 4832 | AUGAAGCU G CUCUGAAA | 2677 | TTTCAGAG GGCTAGCTACAACGA AGCTTCAT | 7620 |
| 4840 | GCUCUGAA G UCUCCUUU | 3029 | AAAGGAGA GGCTAGCTACAACGA TTCAGAGC | 7621 |
| 4850 | CUCCUUUA G CCAUCACC | 2678 | GGTGATGG GGCTAGCTACAACGA TAAAGGAG | 7622 |
| 4853 | CUUUAGCC G UCACCCCA | 1975 | TGGGGTGA GGCTAGCTACAACGA GGCTAAAG | 7623 |
| 4856 | UAGCCAUC G CCCCAACC | 1976 | GGTTGGGG GGCTAGCTACAACGA GATGGCTA | 7624 |
| 4862 | UCACCCCA G CCCCCCAA | 3030 | TTGGGGGG GGCTAGCTACAACGA TGGGGTGA | 7625 |
| 4872 | CCCCCAAA G UUAGUUUG | 3031 | CAAACTAA GGCTAGCTACAACGA TTTGGGGG | 7626 |
| 4876 | CAAAAUUA G UUUGUGUU | 2679 | AACACAAA GGCTAGCTACAACGA TAATTTTG | 7627 |
| 4880 | AUUAGUUU G UGUUACUU | 2680 | AAGTAACA GGCTAGCTACAACGA AAACTAAT | 7628 |
| 4882 | UAGUUUGU G UUACUUAU | 2681 | ATAAGTAA GGCTAGCTACAACGA ACAAACTA | 7629 |
| 4885 | UUUGUGUU G CUUAUGGA | 656 | TCCATAAG GGCTAGCTACAACGA AACACAAA | 7630 |
| 4889 | UGUUACUU G UGGAAGAU | 658 | ATCTTCCA GGCTAGCTACAACGA AAGTAACA | 7631 |
| 4896 | UAUGGAAG G UAGUUUUC | 3032 | GAAAACTA GGCTAGCTACAACGA CTTCCATA | 7632 |
| 4899 | GGAAGAUA G UUUUCUCC | 2682 | GGAGAAAA GGCTAGCTACAACGA TATCTTCC | 7633 |
| 4912 | CUCCUUUU G CUUCACUU | 668 | AAGTGAAG GGCTAGCTACAACGA AAAAGGAG | 7634 |
| 4917 | UUUACUUC G CUUCAAAA | 1992 | TTTTGAAG GGCTAGCTACAACGA GAAGTAAA | 7635 |
| 4926 | CUUCAAAA G CUUUUUAC | 2683 | GTAAAAAG GGCTAGCTACAACGA TTTTGAAG | 7636 |
| 4933 | AGCUUUUU G CUCAAAGA | 677 | TCTTTGAG GGCTAGCTACAACGA AAAAAGCT | 7637 |
| 4942 | CUCAAAGA G UAUAUGUU | 2684 | AACATATA GGCTAGCTACAACGA TCTTTGAG | 7638 |
| 4944 | CAAAGAGU G UAUGUUCC | 679 | GGAACATA GGCTAGCTACAACGA ACTCTTTG | 7639 |
| 4946 | AAGAGUAU G UGUUCCCU | 680 | AGGGAACA GGCTAGCTACAACGA ATACTCTT | 7640 |
| 4948 | GAGUAUAU G UUCCCUCC | 2685 | GGAGGGAA GGCTAGCTACAACGA ATATACTC | 7641 |
| 4959 | CCCUCCAG G UCAGCUGC | 2686 | GCAGCTGA GGCTAGCTACAACGA CTGGAGGG | 7642 |
| 4963 | CCAGGUCA G CUGCCCCC | 2687 | GGGGGCAG GGCTAGCTACAACGA TGACCTGG | 7643 |
| 4966 | GGUCAGCU G CCCCCAAA | 2688 | TTTGGGGG GGCTAGCTACAACGA AGCTGACC | 7644 |
| 4974 | GCCCCCAA G CCCCCUCC | 3033 | GGAGGGGG GGCTAGCTACAACGA TTGGGGGC | 7645 |
| 4985 | CCCUCCUU G CGCUUUGU | 687 | ACAAAGCG GGCTAGCTACAACGA AAGGAGGG | 7646 |
| 4987 | CUCCUUAC G CUTUGUCA | 2689 | TGACAAAG GGCTAGCTACAACGA GTAAGGAG | 7647 |
| 4992 | UACGCUUU G UCACACAA | 2690 | TTGTGTGA GGCTAGCTACAACGA AAAGCGTA | 7648 |
| 4995 | GCUUUGUC G CACAAAAA | 2018 | TTTTTGTG GGCTAGCTACAACGA GACAAAGC | 7649 |
| 4997 | UUUGUCAC G CAAAAAGU | 2019 | ACTTTTTG GGCTAGCTACAACGA GTGACAAA | 7650 |
| 5004 | CACAAAAA G UGUCUCUG | 2691 | CAGAGACA GGCTAGCTACAACGA TTTTTGTG | 7651 |
| 5006 | CAAAAAGU G UCUCUGCC | 2692 | GGCAGAGA GGCTAGCTACAACGA ACTTTTTG | 7652 |
| 5012 | GUGUCUCU G CCUUGAGU | 2693 | ACTCAAGG GGCTAGCTACAACGA AGAGACAC | 7653 |
| 5019 | UGCCUUGA G UCAUCUAU | 2694 | ATAGATGA GGCTAGCTACAACGA TCAAGGCA | 7654 |
| 5022 | CUUGAGUC G UCUAUUCA | 2025 | TGAATAGA GGCTAGCTACAACGA GACTCAAG | 7655 |
| 5026 | AGUCAUCU G UUCAAGCA | 696 | TGCTTGAA GGCTAGCTACAACGA AGATGACT | 7656 |
| 5032 | CUAUUCAA G CACUUACA | 2695 | TGTAAGTG GGCTAGCTACAACGA TTGAATAG | 7657 |
| 5034 | AUUCAAGC G CUUACAGC | 2028 | GCTGTAAG GGCTAGCTACAACGA GCTTGAAT | 7658 |
| 5038 | AAGCACUU G CAGCUCUG | 700 | CAGAGCTG GGCTAGCTACAACGA AAGTGCTT | 7659 |
| 5041 | CACUUACA G CUCUGGCC | 2696 | GGCCAGAG GGCTAGCTACAACGA TGTAAGTG | 7660 |
| 5047 | CAGCUCUG G CCACAACA | 2697 | TGTTGTGG GGCTAGCTACAACGA CAGAGCTG | 7661 |
| 5050 | CUCUGGCC G CAACAGGG | 2034 | CCCTGTTG GGCTAGCTACAACGA GGCCAGAG | 7662 |
| 5053 | UGGCCACA G CAGGGCAU | 3034 | ATGCCCTG GGCTAGCTACAACGA TGTGGCCA | 7663 |
| 5058 | ACAACAGG G CAUUUUAC | 2698 | GTAAAATG GGCTAGCTACAACGA CCTGTTGT | 7664 |
| 5060 | AACAGGGC G UUUUACAG | 2037 | CTGTAAAA GGCTAGCTACAACGA GCCCTGTT | 7665 |
| 5065 | GGCAUUUU G CAGGUGCG | 705 | CGCACCTG GGCTAGCTACAACGA AAAATGCC | 7666 |
| 5069 | UTUUACAG G UGCGAAUG | 2699 | CATTCGCA GGCTAGCTACAACGA CTGTAAAA | 7667 |
| 5071 | UUACAGGU G CGAAUGAC | 2700 | GTCATTCG GGCTAGCTACAACGA ACCTGTAA | 7668 |
| 5075 | AGGUGCGA G UGACAGUA | 3035 | TACTGTCA GGCTAGCTACAACGA TCGCACCT | 7669 |
| 5078 | UGCGAAUG G CAGUAGCA | 3036 | TGCTACTG GGCTAGCTACAACGA CATTCGCA | 7670 |
| 5081 | GAAUGACA G UAGCAUUA | 2701 | TAATGCTA GGCTAGCTACAACGA TGTCATTC | 7671 |
| 5084 | UGACAGUA G CAUUAUGA | 2702 | TCATAATG GGCTAGCTACAACGA TACTGTCA | 7672 |
| 5086 | ACAGUAGC G UUAUGAGU | 2040 | ACTCATAA GGCTAGCTACAACGA GCTACTGT | 7673 |
| 5089 | GUAGCAUU G UGAGUAGU | 708 | ACTACTCA GGCTAGCTACAACGA AATGCTAC | 7674 |
| 5093 | CAUUAUGA G UAGUGUGA | 2703 | TCACACTA GGCTAGCTACAACGA TCATAATG | 7675 |
| 5096 | UAUGAGUA G UGUGAAUU | 2704 | AATTCACA GGCTAGCTACAACGA TACTCATA | 7676 |
| 5098 | UGAGUAGU G UGAAUUCA | 2705 | TGAATTCA GGCTAGCTACAACGA ACTACTCA | 7677 |
| 5102 | UAGUGUGA G UUCAGGUA | 3037 | TACCTGAA GGCTAGCTACAACGA TCACACTA | 7678 |
| 5108 | GAAUUCAG G UAGUAAAU | 2706 | ATTTACTA GGCTAGCTACAACGA CTGAATTC | 7679 |
| 5111 | UUCAGGUA G UAAAUAUG | 2707 | CATATTTA GGCTAGCTACAACGA TACCTGAA | 7680 |
| 5115 | GGUAGUAA G UAUGAAAC | 3038 | GTTTCATA GGCTAGCTACAACGA TTACTACC | 7681 |
| 5117 | UAGUAAAU G UGAAACUA | 714 | TAGTTTCA GGCTAGCTACAACGA ATTTACTA | 7682 |
| 5122 | AAUAUGAA G CUAGGGUU | 3039 | AACCCTAG GGCTAGCTACAACGA TTCATATT | 7683 |
| 5128 | AAACUAGG G UUUGAAAU | 2708 | ATTTCAAA GGCTAGCTACAACGA CCTAGTTT | 7684 |
| 5135 | GGUUUGAA G UUGAUAAU | 3040 | ATTATCAA GGCTAGCTACAACGA TTCAAACC | 7685 |
| 5139 | UGAAAUUG G UAAUGCUU | 3041 | AAGCATTA GGCTAGCTACAACGA CAATTTCA | 7686 |
| 5142 | AAUUGAUA G UGCUUUCA | 3042 | TGAAAGCA GGCTAGCTACAACGA TATCAATT | 7687 |
| 5144 | UUGAUAAU G CUUUCACA | 2709 | TGTGAAAG GGCTAGCTACAACGA ATTATCAA | 7688 |
| 5150 | AUGCUUUC G CAACAUUU | 2044 | AAATGTTG GGCTAGCTACAACGA GAAAGCAT | 7689 |
| 5153 | CUUUCACA G CAUUUGCA | 3043 | TGCAAATG GGCTAGCTACAACGA TGTGAAAG | 7690 |
| 5155 | UUCACAAC G UUUGCAGA | 2046 | TCTGCAAA GGCTAGCTACAACGA GTTGTGAA | 7691 |
| 5159 | CAACAUUU G CAGAUGUU | 2710 | AACATCTG GGCTAGCTACAACGA AAATGTTG | 7692 |
| 5163 | AUUUGCAG G UGUUUUAG | 3044 | CTAAAACA GGCTAGCTACAACGA CTGCAAAT | 7693 |
| 5165 | UUGCAGAU G UUUUAGAA | 2711 | TTCTAAAA GGCTAGCTACAACGA ATCTGCAA | 7694 |
| 5182 | GGAAAAAA G UUCCUUCC | 2712 | GGAAGGAA GGCTAGCTACAACGA TTTTTTCC | 7695 |
| 5195 | UUCCUAAA G UAAUUUCU | 3045 | AGAAATTA GGCTAGCTACAACGA TTTAGGAA | 7696 |
| 5198 | CUAAAAUA G UUUCUCUA | 3046 | TAGAGAAA GGCTAGCTACAACGA TATTTTAG | 7697 |
| 5206 | AUUUCUCU G CAAUUGGA | 739 | TCCAATTG GGCTAGCTACAACGA AGAGAAAT | 7698 |
| 5209 | UCUCUACA G UUGGAAGA | 3047 | TCTTCCAA GGCTAGCTACAACGA TGTAGAGA | 7699 |
| 5217 | AUUGGAAG G UUGGAAGA | 3048 | TCTTCCAA GGCTAGCTACAACGA CTTCCAAT | 7700 |
| 5225 | AUUGGAAG G UUCAGCUA | 3049 | TAGCTGAA GGCTAGCTACAACGA CTTCCAAT | 7701 |
| 5230 | AAGAUUCA G CUAGUUAG | 2713 | CTAACTAG GGCTAGCTACAACGA TGAATCTT | 7702 |
| 5234 | UUCAGCUA G UUAGGAGC | 2714 | GCTCCTAA GGCTAGCTACAACGA TAGCTGAA | 7703 |
| 5241 | AGUUAGGA G CCCAUUUU | 2715 | AAAATGGG GGCTAGCTACAACGA TCCTAACT | 7704 |
| 5245 | AGGAGCCC G UUUUUUCC | 2059 | GGAAAAAA GGCTAGCTACAACGA GGGCTCCT | 7705 |
| 5256 | UUUUCCUA G UCUCUGUG | 3050 | CACACAGA GGCTAGCTACAACGA TAGGAAAA | 7706 |
| 5260 | CCUAAUCU G UGUGUGCC | 2716 | GGCACACA GGCTAGCTACAACGA AGATTAGG | 7707 |
| 5262 | UAAUCUGU G UGUGCCCU | 2717 | AGGGCACA GGCTAGCTACAACGA ACAGATTA | 7708 |
| 5264 | AUCUGUGU G UGCCCUGU | 2718 | ACAGGGCA GGCTAGCTACAACGA ACACAGAT | 7709 |
| 5266 | CUGUGUGU G CCCUGUAA | 2719 | TTACAGGG GGCTAGCTACAACGA ACACACAG | 7710 |
| 5271 | UGUGCCCU G UAACCUGA | 2720 | TCAGGTTA GGCTAGCTACAACGA AGGGCACA | 7711 |
| 5274 | GCCCUGUA G CCUGACUG | 3051 | CAGTCAGG GGCTAGCTACAACGA TACAGGGC | 7712 |
| 5279 | GUAACCUG G CUGGUUAA | 3052 | TTAACCAG GGCTAGCTACAACGA CAGGTTAC | 7713 |
| 5283 | CCUGACUG G UUAACAGC | 2721 | GCTGTTAA GGCTAGCTACAACGA CAGTCAGG | 7714 |
| 5287 | ACUGGUUA G CAGCAGUC | 3053 | GACTGCTG GGCTAGCTACAACGA TAACCAGT | 7715 |
| 5290 | GGUUAACA G CAGUCCUU | 2722 | AAGGACTG GGCTAGCTACAACGA TGTTAACC | 7716 |
| 5293 | UAACAGCA G UCCUUUGU | 2723 | ACAAAGGA GGCTAGCTACAACGA TGCTGTTA | 7717 |
| 5300 | AGUCCUUU G UAAACAGU | 2724 | ACTGTTTA GGCTAGCTACAACGA AAAGGACT | 7718 |
| 5304 | CUUUGUAA G CAGUGUUU | 3054 | AAACACTG GGCTAGCTACAACGA TTACAAAG | 7719 |
| 5307 | UGUAAACA G UGUUUUAA | 2725 | TTAAAACA GGCTAGCTACAACGA TGTTTACA | 7720 |
| 5309 | UAAACAGU G UUUUAAAC | 2726 | GTTTAAAA GGCTAGCTACAACGA ACTGTTTA | 7721 |
| 5316 | UGUUUUAA G CUCUCCUA | 3055 | TAGGAGAG GGCTAGCTACAACGA TTAAAACA | 7722 |
| 5325 | CUCUCCUA G UCAAUAUC | 2727 | GATATTGA GGCTAGCTACAACGA TAGGAGAG | 7723 |
| 5329 | CCUAGUCA G UAUCCACC | 3056 | GGTGGATA GGCTAGCTACAACGA TGACTAGG | 7724 |
| 5331 | UAGUCAAU G UCCACCCC | 770 | GGGGTGGA GGCTAGCTACAACGA ATTGACTA | 7725 |
| 5335 | CAAUAUCC G CCCCAUCC | 2080 | GGATGGGG GGCTAGCTACAACGA GGATATTG | 7726 |
| 5340 | UCCACCCC G UCCAAUUU | 2084 | AAATTGGA GGCTAGCTACAACGA GGGGTGGA | 7727 |
| 5345 | CCCAUCCA G UUUAUCAA | 3057 | TTGATAAA GGCTAGCTACAACGA TGGATGGG | 7728 |
| 5349 | UCCAAUUU G UCAAGGAA | 775 | TTCCTTGA GGCTAGCTACAACGA AAATTGGA | 7729 |
| 5361 | AGGAAGAA G UGGUUCAG | 3058 | CTGAACCA GGCTAGCTACAACGA TTCTTCCT | 7730 |
| 5364 | AAGAAAUG G UUCAGAAA | 2728 | TTTCTGAA GGCTAGCTACAACGA CATTTCTT | 7731 |
| 5373 | UUCAGAAA G UAUUUUCA | 3059 | TGAAAATA GGCTAGCTACAACGA TTTCTGAA | 7732 |
| 5375 | CAGAAAAU G UUUUCAGC | 779 | GCTGAAAA GGCTAGCTACAACGA ATTTTCTG | 7733 |
| 5382 | UAUUUUCA G CCUACAGU | 2729 | ACTGTAGG GGCTAGCTACAACGA TGAAAATA | 7734 |
| 5386 | UUCAGCCU G CAGUUAUG | 784 | CATAACTG GGCTAGCTACAACGA AGGCTGAA | 7735 |
| 5389 | AGCCUACA G UUAUGUUC | 2730 | GAACATAA GGCTAGCTACAACGA TGTAGGCT | 7736 |
| 5392 | CUACAGUU G UGUUCAGU | 786 | ACTGAACA GGCTAGCTACAACGA AACTGTAG | 7737 |
| 5394 | ACAGUUAU G UUCAGUCA | 2731 | TGACTGAA GGCTAGCTACAACGA ATAACTGT | 7738 |
| 5399 | UAUGUUCA G UCACACAC | 2732 | GTGTGTGA GGCTAGCTACAACGA TGAACATA | 7739 |
| 5402 | GUUCAGUC G CACACACA | 2094 | TGTGTGTG GGCTAGCTACAACGA GACTGAAC | 7740 |
| 5404 | UCAGUCAC G CACACAUA | 2095 | TATGTGTG GGCTAGCTACAACGA GTGACTGA | 7741 |
| 5406 | AGUCACAC G CACAUACA | 2096 | TGTATGTG GGCTAGCTACAACGA GTGTGACT | 7742 |
| 5408 | UCACACAC G CAUACAAA | 2097 | TTTGTATG GGCTAGCTACAACGA GTGTGTGA | 7743 |
| 5410 | ACACACAC G UACAAAAU | 2098 | ATTTTGTA GGCTAGCTACAACGA GTGTGTGT | 7744 |
| 5412 | ACACACAU G CAAAAUGU | 790 | ACATTTTG GGCTAGCTACAACGA ATGTGTGT | 7745 |
| 5417 | CAUACAAA G UGUUCCUU | 3060 | AAGGAACA GGCTAGCTACAACGA TTTGTATG | 7746 |
| 5419 | UACAAAAU G UUCCUUUU | 2733 | AAAAGGAA GGCTAGCTACAACGA ATTTTGTA | 7747 |
| 5428 | UUCCUUUU G CUUUUAAA | 2734 | TTTAAAAG GGCTAGCTACAACGA AAAAGGAA | 7748 |
| 5437 | CUTUUAAA G UAAUUUUU | 2735 | AAAAATTA GGCTAGCTACAACGA TTTAAAAG | 7749 |
| 5440 | UUAAAGUA G UUUUUGAC | 3061 | GTCAAAAA GGCTAGCTACAACGA TACTTTAA | 7750 |
| 5447 | AAUUUUUG G CUCCCAGA | 3062 | TCTGGGAG GGCTAGCTACAACGA CAAAAATT | 7751 |
| 5455 | ACUCCCAG G UCAGUCAG | 3063 | CTGACTGA GGCTAGCTACAACGA CTGGGAGT | 7752 |
| 5459 | CCAGAUCA G UCAGAGCC | 2736 | GGCTCTGA GGCTAGCTACAACGA TGATCTGG | 7753 |
| 5465 | CAGUCAGA G CCCCUACA | 2737 | TGTAGGGG GGCTAGCTACAACGA TCTGACTG | 7754 |
| 5471 | GAGCCCCU G CAGCAUUG | 808 | CAATGCTG GGCTAGCTACAACGA AGGGGCTC | 7755 |
| 5474 | CCCCUACA G CAUUGUUA | 2738 | TAACAATG GGCTAGCTACAACGA TGTAGGGG | 7756 |
| 5476 | CCUACAGC G UUGUUAAG | 2114 | CTTAACAA GGCTAGCTACAACGA GCTGTAGG | 7757 |
| 5479 | ACAGCAUU G UUAAGAAA | 2739 | TTTCTTAA GGCTAGCTACAACGA AATGCTGT | 7758 |
| 5488 | UUAAGAAA G UAUUUGAU | 2740 | ATCAAATA GGCTAGCTACAACGA TTTCTTAA | 7759 |
| 5490 | AAGAAAGU G UUUGAUUU | 812 | AAATCAAA GGCTAGCTACAACGA ACTTTCTT | 7760 |
| 5495 | AGUAUUUG G UUUUUGUC | 3064 | GACAAAAA GGCTAGCTACAACGA CAAATACT | 7761 |
| 5501 | UGAUUUUU G UCUCAAUG | 2741 | CATTGAGA GGCTAGCTACAACGA AAAAATCA | 7762 |
| 5507 | UUGUCUCA G UGAAAAUA | 3065 | TATTTTCA GGCTAGCTACAACGA TGAGACAA | 7763 |
| 5513 | CAAUGAAA G UAAAACUA | 3066 | TAGTTTTA GGCTAGCTACAACGA TTTCATTG | 7764 |
| 5518 | AAAAUAAA G CUAUAUUC | 3067 | GAATATAG GGCTAGCTACAACGA TTTATTTT | 7765 |
| 5521 | AUAAAACU G UAUUCAUU | 822 | AATGAATA GGCTAGCTACAACGA AGTTTTAT | 7766 |
| 5523 | AAAACUAU G UUCAUUUC | 823 | GAAATGAA GGCTAGCTACAACGA ATAGTTTT | 7767 |

Input Sequence = NM_005228.1. Cut Site = R/Y
Arm Length = 8. Core Sequence = GGCTAGCTACAACGA
NM_005228.1 (Homo sapiens epidermal growth factor receptor (avian erythroblastic leukemia viral (v-erb-h) oncogene homolog) (EGFR), mRNA)

TABLE VII
Human EGFR Receptor Amberzyme and Substrate Sequence

| Pos | Substrate | Seq ID | Amberzyme | Seq ID |
|---|---|---|---|---|
| 9 | GCCGCGCU G CGCCGGAG | 2118 | CUCCGGCG GGAGGAAACUCC CU UCAAGGACAUCGUCCGGG AGCGCGGC | 7768 |
| 11 | CGCGCUGC G CCGGAGUC | 2119 | GACUCCGG GGAGGAAACUCC CU UCAAGGACAUCGUCCGGG GCAGCGCG | 7769 |
| 14 | GCUGCGCC G GAGUCCCG | 3068 | CGGGACUC GGAGGAAACUCC CU UCAAGGACAUCGUCCGGG GGCGCAGC | 7770 |
| 15 | CUGCGCCG G AGUCCCGA | 3069 | UCGGGACU GGAGGAAACUCC CU UCAAGGACAUCGUCCGGG CGGCGCAG | 7771 |
| 17 | GCGCCGGA G UCCCGAGC | 2120 | GCUCGGGA GGAGGAAACUCC CU UCAAGGACAUCGUCCGGG UCCGGCGC | 7772 |
| 22 | GGAGUCCC G AGCUAGCC | 3070 | GGCUAGCU GGAGGAAACUCC CU UCAAGGACAUCGUCCGGG GGGACUCC | 7773 |
| 24 | AGUCCCGA G CUAGCCCC | 2121 | GGGGCUAG GGAGGAAACUCC CU UCAAGGACAUCGUCCGGG UCGGGACU | 7774 |
| 28 | CCGAGCUA G CCCCGGCG | 2122 | CGCCGGGG GGAGGAAACUCC CU UCAAGGACAUCGUCCGGG UAGCUCGG | 7775 |
| 33 | CUAGCCCC G GCGCCGCC | 3071 | GGCGGCGC GGAGGAAACUCC CU UCAAGGACAUCGUCCGGG GGGGCUAG | 7776 |
| 34 | UAGCCCCG G CGCCGCCG | 2123 | CGGCGGCG GGAGGAAACUCC CU UCAAGGACAUCGUCCGGG CGGGGCUA | 7777 |
| 36 | GCCCCGGC G CCGCCGCC | 2124 | GGCGGCGG GGAGGAAACUCC CU UCAAGGACAUCGUCCGGG GCCGGGGC | 7778 |
| 39 | CCGGCGCC G CCGCCGCC | 2125 | GGCGGCGG GGAGGAAACUCC CU UCAAGGACAUCGUCCGGG GGCGCCGG | 7779 |
| 42 | GCGCCGCC G CCGCCCAG | 2126 | CUGGGCGG GGAGGAAACUCC CU UCAAGGACAUCGUCCGGG GGCGGCGC | 7780 |
| 45 | CCGCCGCC G CCCAGACC | 2127 | GGUCUGGG GGAGGAAACUCC CU UCAAGGACAUCGUCCGGG GGCGGCGG | 7781 |
| 50 | GCCGCCCA G ACCGGACG | 3072 | CGUCCGGU GGAGGAAACUCC CU UCAAGGACAUCGUCCGGG UGGGCGGC | 7782 |
| 54 | CCCAGACC G GACGACAG | 3073 | CUGUCGUC GGAGGAAACUCC CU UCAAGGACAUCGUCCGGG GGUCUGGG | 7783 |
| 55 | CCAGACCG G ACGACAGG | 3074 | CCUGUCGU GGAGGAAACUCC CU UCAAGGACAUCGUCCGGG CGGUCUGG | 7784 |
| 58 | GACCGGAC G ACAGGCCA | 3075 | UGGCCUGU GGAGGAAACUCC CU UCAAGGACAUCGUCCGGG GUCCGGUC | 7785 |
| 62 | GGACGACA G GCCACCUC | 3076 | GAGGUGGC GGAGGAAACUCC CU UCAAGGACAUCGUCCGGG UGUCGUCC | 7786 |
| 63 | GACGACAG G CCACCUCG | 2128 | CGAGGUGG GGAGGAAACUCC CU UCAAGGACAUCGUCCGGG CUGUCGUC | 7787 |
| 71 | GCCACCUC G UCGGCGUC | 2129 | GACGCCGA GGAGGAAACUCC CU UCAAGGACAUCGUCCGGG GAGGUGGC | 7788 |
| 74 | ACCUCGUC G GCGUCCGC | 3077 | GCGGACGC GGAGGAAACUCC CU UCAAGGACAUCGUCCGGG GACGAGGU | 7789 |
| 75 | CCUCGUCG G CGUCCGCC | 2130 | GGCGGACG GGAGGAAACUCC CU UCAAGGACAUCGUCCGGG CGACGAGG | 7790 |
| 77 | UCGUCGGC G UCCGCCCG | 2131 | CGGGCGGA GGAGGAAACUCC CU UCAAGGACAUCGUCCGGG GCCGACGA | 7791 |
| 81 | CGGCGUCC G CCCGAGUC | 2132 | GACUCGGG GGAGGAAACUCC CU UCAAGGACAUCGUCCGGG GGACGCCG | 7792 |
| 85 | GUCCGCCC G AGUCCCCG | 3078 | CGGGGACU GGAGGAAACUCC CU UCAAGGACAUCGUCCGGG GGGCGGAC | 7793 |
| 87 | CCGCCCGA G UCCCCGCC | 2133 | GGCGGGGA GGAGGAAACUCC CU UCAAGGACAUCGUCCGGG UCGGGCGG | 7794 |
| 93 | GAGUCCCC G CCUCGCCG | 2134 | CGGCGAGG GGAGGAAACUCC CU UCAAGGACAUCGUCCGGG GGGGACUC | 7795 |
| 98 | CCCGCCUC G CCGCCAAC | 2135 | GUUGGCGG GGAGGAAACUCC CU UCAAGGACAUCGUCCGGG GAGGCGGG | 7796 |
| 101 | GCCUCGCC G CCAACGCC | 2136 | GGCGUUGG GGAGGAAACUCC CU UCAAGGACAUCGUCCGGG GGCGAGGC | 7797 |
| 107 | CCGCCAAC G CCACAACC | 2137 | GGUUGUGG GGAGGAAACUCC CU UCAAGGACAUCGUCCGGG GUUGGCGG | 7798 |
| 119 | CAACCACC G CGCACGGC | 2138 | GCCGUGCG GGAGGAAACUCC CU UCAAGGACAUCGUCCGGG GGUGGUUG | 7799 |
| 121 | ACCACCGC G CACGGCCC | 2139 | GGGCCGUG GGAGGAAACUCC CU UCAAGGACAUCGUCCGGG GCGGUGGU | 7800 |
| 125 | CCGCGCAC G GCCCCCUG | 3079 | CAGGGGGC GGAGGAAACUCC CU UCAAGGACAUCGUCCGGG GUGCGCGG | 7801 |
| 126 | CGCGCACG G CCCCCUGA | 2140 | UCAGGGGG GGAGGAAACUCC CU UCAAGGACAUCGUCCGGG CGUGCGCG | 7802 |
| 133 | GGCCCCCU G ACUCCGUC | 3080 | GACGGAGU GGAGGAAACUCC CU UCAAGGACAUCGUCCGGG AGGGGGCC | 7803 |
| 139 | CUGACUCC G UCCAGUAU | 2141 | AUACUGGA GGAGGAAACUCC CU UCAAGGACAUCGUCCGGG GGAGUCAG | 7804 |
| 144 | UCCGUCCA G UAUUGAUC | 2142 | GAUCAAUA GGAGGAAACUCC CU UCAAGGACAUCGUCCGGG UGGACGGA | 7805 |
| 149 | CCAGUAUU G AUCGGGAG | 3081 | CUCCCGAU GGAGGAAACUCC CU UCAAGGACAUCGUCCGGG AAUACUGG | 7806 |
| 153 | UAUUGAUC G GGAGAGCC | 3082 | GGCUCUCC GGAGGAAACUCC CU UCAAGGACAUCGUCCGGG GAUCAAUA | 7807 |
| 154 | AUUGAUCG G GAGAGCCG | 3083 | CGGCUCUC GGAGGAAACUCC CU UCAAGGACAUCGUCCGGG CGAUCAAU | 7808 |
| 155 | UUGAUCGG G AGAGCCGG | 3084 | CCGGCUCU GGAGGAAACUCC CU UCAAGGACAUCGUCCGGG CCGAUCAA | 7809 |
| 157 | GAUCGGGA G AGCCGGAG | 3085 | CUCCGGCU GGAGGAAACUCC CU UCAAGGACAUCGUCCGGG UCCCGAUC | 7810 |
| 159 | UCGGAGAG G CCGGAGCG | 2143 | CGCUCCGG GGAGGAAACUCC CU UCAAGGACAUCGUCCGGG UCUCCCGA | 7811 |
| 162 | GGAGAGCC G GAGCGAGC | 3086 | GCUCGCUC GGAGGAAACUCC CU UCAAGGACAUCGUCCGGG GGCUCUCC | 7812 |
| 163 | GAGAGCCG G AGCGAGCU | 3087 | AGCUCGCU GGAGGAAACUCC CU UCAAGGACAUCGUCCGGG CGGCUCUC | 7813 |
| 165 | GAGCCGGA G CGAGCUCU | 2144 | AGAGCUCG GGAGGAAACUCC CU UCAAGGACAUCGUCCGGG UCCGGCUC | 7814 |
| 167 | GCCGGAGC G AGCUCUUC | 3088 | GAAGAGCU GGAGGAAACUCC CU UCAAGGACAUCGUCCGGG GCUCCGGC | 7815 |
| 169 | CGGAGCGA G CUCUUCGG | 2145 | CCGAAGAG GGAGGAAACUCC CU UCAAGGACAUCGUCCGGG UCGCUCCG | 7816 |
| 176 | AGCUCUUC G GGGAGCAG | 3089 | CUGCUCCC GGAGGAAACUCC CU UCAAGGACAUCGUCCGGG GAAGAGCU | 7817 |
| 177 | GCUCUUCG G GGAGCAGC | 3090 | GCUGCUCC GGAGGAAACUCC CU UCAAGGACAUCGUCCGGG CGAAGAGC | 7818 |
| 178 | CUCUUCGU G GAGCAUCG | 3091 | CGCUGCUC UGAGGAAACUCC CU UCAAGGACAUCGUCCGGG CCGAAGAG | 7819 |
| 179 | UCUUCGGG G AGCAGCGA | 3092 | UCGCUGCU GGAGGAAACUCC CU UCAAGGACAUCGUCCUGG CCCGAAGA | 7820 |
| 181 | UUCGGGGA G CAGCGAUG | 2146 | CAUCGCUG GGAGGAAACUCC CU UCAAGGACAUCGUCCGGG UCCCCGAA | 7821 |
| 184 | GGGGAGCA G CGAUGCGA | 2147 | UCGCAUCG GGAGGAAACUCC CU UCAAGGACAUCGUCCGGG UGCUCCCC | 7822 |
| 186 | GGAGCAGC G AUGCGACC | 3093 | GGUCGCAU GGAGGAAACUCC CU UCAAGGACAUCGUCCGGG GCUGCUCC | 7823 |
| 189 | GCAGCGAU G CGACCCUC | 2148 | GAGGGUCG GGAGGAAACUCC CU UCAAGGACAUCGUCCGGG AUCGCUGC | 7824 |
| 191 | AGCGAUGC G ACCCUCCG | 3094 | CGGAGGGU GGAGGAAACUCC CU UCAAGGACAUCGUCCGGG GCAUCGCU | 7825 |
| 199 | GACCCUCC G GGACGGCC | 3095 | GGCCGUCC GGAGGAAACUCC CU UCAAGGACAUCGUCCGGG GGAGGGUC | 7826 |
| 200 | ACCCUCCG G GACGGCCG | 3096 | CGGCCGUC GGAGGAAACUCC CU UCAAGGACAUCGUCCGGG CGGAGGGU | 7827 |
| 201 | CCCUCCGU G ACGGCCGG | 3097 | CCGGCCGU GGAUGAAACUCC CU UCAAGGACAUCGUCCGGG CCGGAGGG | 7828 |
| 204 | UCCUGGAC G GCCGGGGC | 3098 | GCCCCUGC GGAGGAAACUCC CU UCAAGGACAUCGUCCGGG GUCCCGGA | 7829 |
| 205 | CCUGGACG G CCGGGGCA | 2149 | UGCCCCGG GGAGGAAACUCC CU UCAAGGACAUCUUCCGGG CGUCCCGG | 7830 |
| 208 | GGACUGCC G GGGCAGCG | 3099 | CGCUGCCC GGAGGAAACUCC CU UCAAGGACAUCGUCCGGU UGCCGUCC | 7831 |
| 209 | GACGGCCG G GGCAGCGC | 3100 | GCGCUGCC GGAGGAAACUCC CU UCAAGGACAUCGUCCGGG CGGCCGUC | 7832 |
| 210 | ACGGCCGG G GCAUCGCU | 3101 | AGCGCUGC GGAGGAAACUCC CU UCAAGGACAUCGUCCGGG CCGGCCGU | 7833 |
| 211 | CGGCCGGG G CAGCGCUC | 2150 | GAUCUCUG GGAGGAAACUCC CU UCAAGGACAUCGUCCGGG CCCGGCCG | 7834 |
| 214 | CCGGGGCA G CGCUCCUG | 2151 | CAGGAGCG GGAGGAAACUCC CU UCAAGGACAUCGUCCGGU UGCCCCGG | 7835 |
| 216 | GGGGCAGC G CUCCUGGC | 2152 | GCCAGGAG GGAUGAAACUCC CU UCAAGGACAUCGUCCGGG GCUGCCCC | 7836 |
| 222 | GCGCUCCU G GCGCUGCU | 3102 | AGCAGCGC GGAGUAAACUCC CU UCAAGGACAUCGUCCUGG AGGAGCGC | 7837 |
| 223 | CGCUCCUG G CGCUGCUG | 2153 | CAGCAGCG GGAGGAAACUCC CU UCAAGGACAUCGUCCGGU CAGGAGCG | 7838 |
| 225 | CUCCUGGC G GUGCUGGC | 2154 | GCCAGCAG GGAGGAAACUCC CU UCAAGGACAUCGUCCGGG GCCAGGAG | 7839 |
| 228 | CUGGCGCU G CUGGCUGC | 2155 | GCAGCCAG GGAGGAAACUCC CU UCAAGGACAUCGUCCGGG AGCGCCAG | 7840 |
| 231 | GCGCUGCU G GCUGCGCU | 3103 | AGCGCAGC GGAGGAAACUCC CU UCAAGGACAUCGUCCGGG AGCAGCGC | 7841 |
| 232 | CGCUGCUG G CUGCGCUC | 2156 | GAGCGCAG GGAGGAAACUCC CU UCAAGGACAUCGUCCGGU CAGCAGCG | 7842 |
| 235 | UGCUGGCU G CGCUCUGC | 2157 | GCAGAGCG GGAUGAAACUCC CU UCAAGGACAUCGUCCGGG AGCCAGCA | 7843 |
| 237 | CUGGCUGC G CUCUGCCC | 2158 | GGGCAGAG GGAGUAAACUCC CU UCAAGGACAUCGUCCUGG GCAGCCAG | 7844 |
| 242 | UGCGCUCU G CCCGGCGA | 2159 | UCGCCGGG GGAGUAAACUCC CU UCAAGGACAUCGUCCUGG AGAGCGCA | 7845 |
| 246 | CUCUGCCC G GCGAGUCG | 3104 | CGACUCGC GGAGGAAACUCC CU UCAAGGACAUCGUCCGGG GGGCAGAG | 7846 |
| 247 | UCUGCCCG G CGAGUCGG | 2160 | CCGACUCG GGAGGAAACUCC CU UCAAGGACAUCGUCCGGG CGGGCAGA | 7847 |
| 249 | UGCCCGGC G AGUCGGGC | 3105 | GCCCGACU GGAGGAAACUCC CU UCAAGGACAUCGUCCGGG GCCGGGCA | 7848 |
| 251 | CCCGGCGA G UCGGGCUC | 2161 | GAGCCCGA GGAGGAAACUCC CU UCAAGGACAUCGUCCGGG UCGCCGGG | 7849 |
| 254 | GGCGAGUC G GGCUCUGG | 3106 | CCAGAGCC GGAGGAAACUCC CU UCAAGGACAUCGUCCGGG GACUCGCC | 7850 |
| 255 | GCGAGUCG G GCUCUGGA | 3107 | UCCAGAGC GGAGGAAACUCC CU UCAAGGACAUCGUCCGGG CGACUCGC | 7851 |
| 256 | CGAGUCGG G CUCUGGAG | 2162 | CUCCAGAG GGAGGAAACUCC CU UCAAGGACAUCGUCCGGG CCGACUCG | 7852 |
| 261 | CGGGCUCU G GAGGAAAA | 3108 | UUUUCCUC GGAGGAAACUCC CU UCAAGGACAUCGUCCGGG AGAGCCCG | 7853 |
| 262 | GGGCUCUG G AGGAAAAG | 3109 | CUUUUCUU GGAGGAAACUCC CU UCAAGGACAUCGUCCGGG CAGAGCCC | 7854 |
| 264 | GCUCUGGA G GAAAAGAA | 3110 | UUCUUUUC GGAGGAAACUCC CU UCAAGGACAUCGUCCGGG UCCAGAGC | 7855 |
| 265 | CUCUGGAG G AAAAGAAA | 3111 | UUUCUUUU GGAGGAAACUCC CU UCAAGGACAUCGUCCGGG CUCCAGAG | 7856 |
| 270 | GAGGAAAA G AAAGUUUG | 3112 | CAAACUUU GGAGGAAACUCC CU UCAAGGACAUCGUCCGGG UUUUCCUC | 7857 |
| 274 | AAAAGAAA G UUUGCCAA | 2163 | UUGGCAAA GGAGGAAACUCC CU UCAAGGACAUCGUCCGGG UUUCUUUU | 7858 |
| 278 | GAAAGUUU G CCAAGGCA | 2164 | UGCCUUGG GGAGGAAACUCC CU UCAAGGACAUCGUCCGGG AAACUUUC | 7859 |
| 283 | UUUGCCAA G GCACGAGU | 3113 | ACUCGUGC GGAGGAAACUCC CU UCAAGGACAUCGUCCGGG UUGGCAAA | 7860 |
| 284 | UUGCCAAG G CACGAGUA | 2165 | UACUCGUG GGAGGAAACUCC CU UCAAGGACAUCGUCCGGG CUUGGCAA | 7861 |
| 288 | CAAGGCAC G AGUAACAA | 3114 | UUGUUACU GGAGGAAACUCC CU UCAAGGACAUCGUCCGGG GUGCCUUG | 7862 |
| 290 | AGGCACGA G UAACAAGC | 2166 | GCUUGUUA GGAGGAAACUCC CU UCAAGGACAUCGUCCGGG UCGUGCCU | 7863 |
| 297 | AGUAACAA G CUCACGCA | 2167 | UGCGUGAG GGAGGAAACUCC CU UCAAGGACAUCGUCCGGG UUGUUACU | 7864 |
| 303 | AAGCUCAC G CAGUUGGG | 2168 | CCCAACUG GGAGGAAACUCC CU UCAAGGACAUCGUCCGGG GUGAGCUU | 7865 |
| 306 | CUCACGCA G UUGGGCAC | 2169 | GUGCCCAA GGAGGAAACUCC CU UCAAGGACAUCGUCCGGG UGCGUGAG | 7866 |
| 309 | ACGCAGUU G GGCACUUU | 3115 | AAAGUGCC GGAGGAAACUCC CU UCAAGGACAUCGUCCGGG AACUGCGU | 7867 |
| 310 | CGCAGUUG G GCACUUUU | 3116 | AAAAGUGC GGAGGAAACUCC CU UCAAGGACAUCGUCCGGG CAACUGCG | 7868 |
| 311 | GCAGUUGG G CACUUUUG | 2170 | CAAAAGUG GGAGGAAACUCC CU UCAAGGACAUCGUCCGGG CCAACUGC | 7869 |
| 319 | GCACUUUU G AAGAUCAU | 3117 | AUGAUCUU GGAGGAAACUCC CU UCAAGGACAUCGUCCGGG AAAAGUGC | 7870 |
| 322 | CUUUUGAA G AUCAUUUU | 3118 | AAAAUGAU GGAGGAAACUCC CU UCAAGGACAUCGUCCGGG UUCAAAAG | 7871 |
| 335 | UUUUCUCA G CCUCCAGA | 2171 | UCUGGAGG GGAGGAAACUCC CU UCAAGGACAUCGUCCGGG UGAGAAAA | 7872 |
| 342 | AGCCUCCA G AGGAUGUU | 3119 | AACAUCCU GGAGGAAACUCC CU UCAAGGACAUCGUCCGGG UGGAGGCU | 7873 |
| 344 | CCUCCAGA G GAUGUUCA | 3120 | UGAACAUC GGAGGAAACUCC CU UCAAGGACAUCGUCCGGG UCUGGAGG | 7874 |
| 345 | CUCCAGAG G AUGUUCAA | 3121 | UUGAACAU GGAGGAAACUCC CU UCAAGGACAUCGUCCGGG CUCUGGAG | 7875 |
| 348 | CAGAGGAU G UUCAAUAA | 2172 | UUAUUGAA GGAGGAAACUCC CU UCAAGGACAUCGUCCGGG AUCCUCUG | 7876 |
| 359 | CAAUAACU G UGAGGUGG | 2173 | CCACCUCA GGAGGAAACUCC CU UCAAGGACAUCGUCCGGG AGUUAUUG | 7877 |
| 361 | AUAACUGU G AGGUGGUC | 3122 | GACCACCU GGAGGAAACUCC CU UCAAGGACAUCGUCCGGG ACAGUUAU | 7878 |
| 363 | AACUGUGA G GUGGUCCU | 3123 | AGGACCAC GGAGGAAACUCC CU UCAAGGACAUCGUCCGGG UCACAGUU | 7879 |
| 364 | ACUGUGAG G UGGUCCUU | 2174 | AAGGACCA GGAGGAAACUCC CU UCAAGGACAUCGUCCGGG CUCACAGU | 7871 |
| 366 | UGUGAGGU G GUCCUUGG | 3124 | CCAAGGAC GGAGGAAACUCC CU UCAAGGACAUCGUCCGGG ACCUCACA | 7881 |
| 367 | GUGAGGUG G UCCUUGGG | 2175 | CCCAAGGA GGAGGAAACUCC CU UCAAGGACAUCGUCCGGG CACCUCAC | 7882 |
| 373 | UGGUCCUU G GGAAUUUG | 3125 | CAAAUUCC GGAGGAAACUCC CU UCAAGGACAUCGUCCGGG AAGGACCA | 7883 |
| 374 | GGUCCUUG G GAAUUUGG | 3126 | CCAAAUUC GGAGGAAACUCC CU UCAAGGACAUCGUCCGGG CAAGGACC | 7884 |
| 375 | GUCCUUGG G AAUUUGGA | 3127 | UCCAAAUU GGAGGAAACUCC CU UCAAGGACAUCGUCCGGG CCAAGGAC | 7885 |
| 381 | GGGAAUUU G GAAAUUAC | 3128 | GUAAUUUC GGAGGAAACUCC CU UCAAGGACAUCGUCCGGG AAAUUCCC | 7886 |
| 382 | GGAAUUUG G AAAUUACC | 3129 | GGUAAUUU GGAGGAAACUCC CU UCAAGGACAUCGUCCGGG CAAAUUCC | 7887 |
| 394 | UUACCUAU G UGCAGAGG | 2176 | CCUCUGCA GGAGGAAACUCC CU UCAAGGACAUCGUCCGGG AUAGGUAA | 7888 |
| 396 | ACCUAUGU G CACACGAA | 2177 | UUCCUCUG GGAGGAAACUCC CU UCAAGGACAUCGUCCGGG ACAUAGGU | 7889 |
| 399 | UAUGUGCA G AGGAAUUA | 3130 | UAAUUCCU GGAGGAAACUCC CU UCAAGGACAUCGUCCGGG UGCACAUA | 7890 |
| 401 | UGUGCAGA G GAAUUAUG | 3131 | CAUAAUUC GGAGGAAACUCC CU UCAAGGACAUCGUCCGGG UCUGCACA | 7891 |
| 402 | GUGCAGAG G AAUUAUGA | 3132 | UCAUAAUU GGAGGAAACUCC CU UCAAGGACAUCGUCCGGG CUCUGCAC | 7892 |
| 409 | GGAAUUAU G AUCUUUCC | 3133 | GGAAAGAU GGAGGAAACUCC CU UCAAGGACAUCGUCCGGG AUAAUUCC | 7893 |
| 426 | UUCUUAAA G ACCAUCCA | 3134 | UGGAUGGU GGAGGAAACUCC CU UCAAGGACAUCGUCCGGG UUUAAGAA | 7894 |
| 435 | ACCAUCCA G GAGGUGGC | 3135 | GCCACCUC GGAGGAAACUCC CU UCAAGGACAUCGUCCGGG UGGAUGGU | 7895 |
| 436 | CCAUCCAG G AGGUGGCU | 3136 | AGCCACCU GGAGGAAACUCC CU UCAAGGACAUCGUCCGGG CUGGAUGG | 7896 |
| 438 | AUCCAGGA G GUGGCUGG | 3137 | CCAGCCAC GGAGGAAACUCC CU UCAAGGACAUCGUCCGGG UCCUGGAU | 7897 |
| 439 | UCCAGGAG G UGGCUGGU | 2178 | ACCAGCCA GGAGGAAACUCC CU UCAAGGACAUCGUCCGGG CUCCUGGA | 7898 |
| 441 | CAGGAGGU G GCUGGUUA | 3138 | UAACCAGC GGAGGAAACUCC CU UCAAGGACAUCGUCCGGG ACCUCCUG | 7899 |
| 442 | AGGAGGUG G CUGGUUAU | 2179 | AUAACCAG GGAGGAAACUCC CU UCAAGGACAUCGUCCGGG CACCUCCU | 7900 |
| 445 | AGGUGGCU G GUUAUGUC | 3139 | GACAUAAC GGAGGAAACUCC CU UCAAGGACAUCGUCCGGG AGCCACCU | 7901 |
| 446 | GGUGGCUG G UUAUGUCC | 2180 | GGACAUAA GGAGGAAACUCC CU UCAAGGACAUCGUCCGGG CAGCCACC | 7902 |
| 451 | CUGGUUAU G UCCUCAUU | 2181 | AAUGAGGA GGAGGAAACUCC CU UCAAGGACAUCGUCCGGG AUAACCAG | 7903 |
| 460 | UCCUCAUU G CCCUCAAC | 2182 | GUUGAGGG GGAGGAAACUCC CU UCAAGGACAUCGUCCGGG AAUGAGGA | 7904 |
| 472 | UCAACACA G UGGAGCGA | 2183 | UCGCUCCA GGAGGAAACUCC CU UCAAGGACAUCGUCCGGG UGUGUUGA | 7905 |
| 474 | AACACAGU G GAGCGAAU | 3140 | AUUCGCUC GGAGGAAACUCC CU UCAAGGACAUCGUCCGGG ACUGUGUU | 7906 |
| 475 | ACACAGUG G AGCGAAUU | 3141 | AAUUCGCU GGAGGAAACUCC CU UCAAGGACAUCGUCCGGG CACUGUGU | 7907 |
| 477 | ACAGUGGA G CGAAUUCC | 2184 | GGAAUUCG GGAGGAAACUCC CU UCAAGGACAUCGUCCGGG UCCACUGU | 7908 |
| 479 | AGUGGAGC G AAUUCCUU | 3142 | AAGGAAUU GGAGGAAACUCC CU UCAAGGACAUCGUCCGGG GCUCCACU | 7909 |
| 489 | AUUCCUUU G GAAAACCU | 3143 | AGGUUUUC GGAGGAAACUCC CU UCAAGGACAUCGUCCGGG AAAGGAAU | 7910 |
| 490 | UUCCUUUG G AAAACCUG | 3144 | CAGGUUUU GGAGGAAACUCC CU UCAAGGACAUCGUCCGGG CAAAGGAA | 7911 |
| 498 | GAAAACCU G UCCUCAUU | 2185 | AUGAUCUG GGAGGAAACUCC CU UCAAGGACAUCGUCCGGG AGGUUUUC | 7912 |
| 501 | AACCUGGA G CCCUCAAC | 3145 | CUGAUGAU GGAGGAAACUCC CU UCAAGGACAUCGUCCGGG UGCAGGUU | 7913 |
| 509 | GAUCAUCA G AGGAAAUA | 3146 | UAUUUCCU GGAGGAAACUCC CU UCAAGGACAUCGUCCGGG UGAUGAUC | 7914 |
| 511 | UCAUCAGA G GAAAUAUG | 3147 | CAUAUUUC GGAGGAAACUCC CU UCAAGGACAUCGUCCGGG UCUGAUGA | 7915 |
| 512 | CAUCAGAG G AAAUAUGU | 3148 | ACAUAUUU GGAGGAAACUCC CU UCAAGGACAUCGUCCGGG CUCUGAUG | 7916 |
| 519 | GGAAAUAU G UACUACGA | 2184 | UCGUAGUA GGAGGAAACUCC CU UCAAGGACAUCGUCCGGG AUAUUUCC | 7917 |
| 526 | UGUACUAC G AAAAUUCC | 3149 | GGAAUUUU GGAGGAAACUCC CU UCAAGGACAUCGUCCGGG GUAGUACA | 7918 |
| 538 | AUUCCUAU G CCUUAGCA | 2187 | UGCUAAGG GGAGGAAACUCC CU UCAAGGACAUCGUCCGGG AUGAGGAU | 7919 |
| 544 | AUGCCUUA G CAGUCUUA | 2188 | UAAGACUG GGAGGAAACUCC CU UCAAGGACAUCGUCCGGG UAAGGCAU | 7920 |
| 547 | CCUUAGCA G UCUUAUCU | 2189 | AGAUAAGA GGAGGAAACUCC CU UCAAGGACAUCGUCCGGG UGCUAAGG | 7921 |
| 562 | CUAACUAU G AUGCAAAU | 3150 | AUUUGCAU GGAGGAAACUCC CU UCAAGGACAUCGUCCGGG AUAGUUAG | 7922 |
| 565 | ACUAUGAU G CAAAUAAA | 2190 | UUUAUUUG GGAGGAAACUCC CU UCAAGGACAUCGUCCGGG AUCAUAGU | 7923 |
| 577 | AUAAAACC G GACUGAAG | 3151 | CUUCAGUC GGAGGAAACUCC CU UCAAGGACAUCGUCCGGG GGUUUUAU | 7924 |
| 578 | UAAAACCG G ACUGAAGG | 3152 | CCUUCAGU GGAGGAAACUCC CU UCAAGGACAUCGUCCGGG CGGUUUUA | 7925 |
| 582 | ACCGGACU G AAGGAGCU | 3153 | AGCUCCUU GGAGGAAACUCC CU UCAAGGACAUCGUCCGGG AGUCCGGU | 7926 |
| 585 | GGACUGAA G GAGCUGCC | 3154 | GGCAGCUC GGAGGAAACUCC CU UCAAGGACAUCGUCCGGG UUCAGUCC | 7927 |
| 586 | GACUGAAG G AGCUGCCC | 3155 | GGGCAGCU GGAGGAAACUCC CU UCAAGGACAUCGUCCGGG CUUCAGUC | 7928 |
| 588 | CUGAAGGA G CUGCCCAU | 2191 | AUGCGCAG GGACGAAACUCC CU UCAAGGACAUCGUCCGGG UCCUUCAG | 7929 |
| 591 | AAGGAGCU G CCCAUGAG | 2192 | CUCAUGGG GGAGGAAACUCC CU UCAAGGACAUCGUCCGGG AGCUCCUU | 7930 |
| 597 | CUGCCCAU G AGAAAUUU | 3156 | AAAUUUCU GGACGAAACUCC CU UCAAGGACAUCGUCCGGG AUGGGCAG | 7931 |
| 599 | GCCCAUGA G AAAUUUAC | 3157 | GUAAAUUU GGAGGAAACUCC CU UCAAGGACAUCGUCCGGG UCAUGGGC | 7932 |
| 609 | AAUUUACA G GAAAUCCU | 3158 | AGGAUUUC GGAGGAAACUCC CU UCAAGGACAUCGUCCGGG UGUAAAUU | 7933 |
| 610 | AUUUACAC G AAAUCCUG | 3159 | CAGGAUUU GGAGGAAACUCC CU UCAAGGACAUCGUCCGGG CUGUAAAU | 7934 |
| 618 | GAAAUCCU G CAUGGCGC | 2193 | GCGCCAUG GGAGGAAACUCC CU UCAAGGACAUCGUCCGCG AGGAUUUC | 7935 |
| 622 | UCCUGCAU G GCGCCGUG | 3160 | CACGGCGC GGAGGAAACUCC CU UCAAGGACAUCGUCCGGG AUGCAGGA | 7936 |
| 623 | CCUGCAUG G CCCCGUCC | 2194 | GCACGGCG GGAGGAAACUCC CU UCAAGGACAUCGUCCGGG CAUGCAGG | 7937 |
| 625 | UGCAUGGC G CCGUGCGG | 2195 | CCGCACGG GGAGGAAACUCC CU UCAAGGACAUCGUCCGGG GCCAUGCA | 7938 |
| 628 | AUGGCGCC G UCCCGUUC | 2196 | GAACCGCA GGAGGAAACUCC CU UCAAGCACAUCGUCCGGG GGCGCCAU | 7939 |
| 630 | GGCGCCGU G CGGUUCAG | 2197 | CUGAACCG GGAGGAAACUCC CU UCAAGGACAUCGUCCGGG ACGGCGCC | 7940 |
| 632 | CGCCGUGC G GUUCAGCA | 3161 | UGCUGAAC CGAGGAAACUCC CU UCAAGGACAUCGUCCGCC GCACGGCG | 7941 |
| 633 | GCCGUGCG G UUCAGCAA | 2198 | UUGCUGAA GGAGGAAACUCC CU UCAAGGACAUCGUCCGGG CGCACGGC | 7942 |
| 638 | GCGGUUCA G CAACAACC | 2199 | GGUUGUUG CGAGGAAACUCC CU UCAAGGACAUCGUCCCGG UGAACCGC | 7943 |
| 649 | ACAACCCU G CCCUGUGC | 2200 | GCACAGGG GGAGGAAACUCC CU UCAAGGACAUCGUCCGGG AGGGUUGU | 7944 |
| 654 | CCUGCCCU G UCCAACCU | 2201 | ACCUUGCA GGAGGAAACUCC CU UCAAGGACAUCCUCCGCG AGGGCAGG | 7945 |
| 656 | UGCCCUGU G CAACCUGG | 2202 | CCACCUUG CGACCAAACUCC CU UCAACGACAUCGUCCGCC ACAGGGCA | 7946 |
| 661 | UCUGCAAC G UGCACACC | 2203 | GCUCUCCA CGACGAAACUCC CU UCAAGCACAUCGUCCCGC GUUGCACA | 7947 |
| 663 | UCCAACGU G GAGAGCAU | 3162 | AUGCUCUC GGACGAAACUCC CU UCAACCACAUCGUCCGCG ACGUUGCA | 7948 |
| 664 | GCAACGUG G AGAGCAUC | 3163 | GAUGCUCU GGAGGAAACUCC CU UCAAGGACAUCGUCCGGG CACGUUGC | 7949 |
| 666 | AACGUGGA G AGCAUCCA | 3164 | UGGAUGCU CGAGGAAACUCC CU UCAAGGACAUCGUCCGCC UCCACGUU | 7950 |
| 668 | CGUGGAGA G CAUCCAGU | 2204 | ACUGGAUG GGAGGAAACUCC CU UCAAGGACAUCGUCCGGG UCUCCACG | 7951 |
| 675 | AGCAUCCA G UGGCGGGA | 2205 | UCCCGCCA CGAGGAAACUCC CU UCAAGGACAUCGUCCCGG UGGAUGCU | 7952 |
| 677 | CAUCCAGU G GCGGGACA | 3165 | UGUCCCGC GGAGGAAACUCC CU UCAAGGACAUCGUCCGGG ACUGGAUG | 7953 |
| 678 | AUCCAGUG G CGGGACAU | 2206 | AUGUCCCG GGAGGAAACUCC CU UCAAGGACAUCCUCCGCG CACUGGAU | 7954 |
| 680 | CCAGUGGC G GGACAUAG | 3166 | CUAUGUCC CGACCAAACUCC CU UCAACGACAUCGUCCGCC GCCACUGG | 7955 |
| 681 | CAGUGGCG G GACAUAGU | 3167 | ACUAUGUC CGACGAAACUCC CU UCAAGCACAUCGUCCCGC CGCCACUG | 7956 |
| 682 | AGUGGCGG G ACAUAGUC | 3168 | GACUAUGU GGACGAAACUCC CU UCAACCACAUCGUCCGCG CCGCCACU | 7957 |
| 688 | GGGACAUA G UCAGCAGU | 2207 | ACUGCUGA GGAGGAAACUCC CU UCAAGGACAUCGUCCGGG UAUGUCCC | 7958 |
| 692 | CAUAGUCA G CAGUGACU | 2208 | AGUCACUG GGAGGAAACUCC CU UCAAGGACAUCGUCCGGG UGACUAUG | 7959 |
| 695 | AGUCAGCA G UGACUUUC | 2209 | GAAAGUCA GGAGGAAACUCC CU UCAAGGACAUCGUCCGGG UGCUGACU | 7960 |
| 697 | UCAGCAGU G ACUUUCUC | 3169 | GAGAAAGU GGAGGAAACUCC CU UCAAGGACAUCGUCCGGG ACUGCUGA | 7961 |
| 707 | CUUUCUCA G CAACAUGU | 2210 | ACAUGUUG GGAGGAAACUCC CU UCAAGGACAUCGUCCGGG UGAGAAAG | 7962 |
| 714 | AGCAACAU G UCCAUGGA | 2211 | UCCAUCGA GGAGGAAACUCC CU UCAAGGACAUCGUCCGGG AUGUUGCU | 7963 |
| 717 | AACAUGUC G AUGGACUU | 3170 | AAGUCCAU GGAGGAAACUCC CU UCAAGGACAUCGUCCGGG GACAUGUU | 7964 |
| 720 | AUGUCGAU G GACUUCCA | 3171 | UGGAAGUC GGAGGAAACUCC CU UCAAGGACAUCGUCCGGG AUCGACAU | 7965 |
| 721 | UGUCGAUG G ACUUCCAG | 3172 | CUGGAAGU GGAGGAAACUCC CU UCAAGGACAUCGUCCGGG CAUCGACA | 7966 |
| 729 | GACUUCCA G AACCACCU | 3173 | AGGUGGUU GGAGGAAACUCC CU UCAAGGACAUCGUCCGGG UGGAAGUC | 7967 |
| 738 | AACCACCU G GGCAGCUG | 3174 | CAGCUGCC GGAGGAAACUCC CU UCAAGGACAUCGUCCGGG AGGUGGUU | 7968 |
| 739 | ACCACCUG G GCAGCUGC | 3175 | GCAGCUGC GGAGGAAACUCC CU UCAAGGACAUCGUCCGGG CAGGUGGU | 7969 |
| 740 | CCACCUGG G CAGCUGCC | 2212 | GGCAGCUG GGAGGAAACUCC CU UCAAGGACAUCGUCCGGG CCAGGUGG | 7970 |
| 743 | CUGGGCAG G CUGCCAAA | 2213 | UUUGGCAG GGAGGAAACUCC CU UCAAGGACAUCGUCCGGG UGCCCAGG | 7971 |
| 746 | GGGCAGCU G CCAAAAGU | 2214 | ACUUUUGG GGAGGAAACUCC CU UCAAGGACAUCGUCCGGG AGCUGCCC | 7972 |
| 753 | UGCCAAAA G UGUGAUCC | 2215 | GGAUCACA GGAGGAAACUCC CU UCAAGGACAUCGUCCGGG UUUUGGCA | 7973 |
| 755 | CCAAAAGU G UGAUCCAA | 2216 | UUGGAUCA GGAGGAAACUCC CU UCAAGGACAUCGUCCGGG ACUUUUGG | 7974 |
| 757 | AAAAGUGU G AUCCAAGC | 3176 | GCUUGGAU GGAGGAAACUCC CU UCAAGGACAUCGUCCGGG ACACUUUU | 7975 |
| 764 | UGAUCCAA G CUGUCCCA | 2217 | UGUGACAG GGAGGAAACUCC CU UCAAGGACAUCGUCCGGG UUGGAUCA | 7976 |
| 767 | UCCAAGCU G UCCCAAUG | 2218 | CAUUGGGA GGAGGAAACUCC CU UCAAGGACAUCGUCCGGG AGCUUGGA | 7977 |
| 775 | GUCCCAAU G GGAGCUGC | 3177 | GCAGCUCC GGAGGAAACUCC CU UCAAGGACAUCGUCCGGG AUUGGGAC | 7978 |
| 776 | UCCCAAUG G GAGCUGCU | 3178 | AGCAGCUC GGAGGAAACUCC CU UCAAGGACAUCGUCCGGG CAUUGGGA | 7979 |
| 777 | CCCAAUGG G AGCUGCUG | 3179 | CAGCAGCU GGAGGAAACUCC CU UCAAGGACAUCGUCCGGG CCAUUGGG | 7980 |
| 779 | CAAUGGGA G CUGCUGGG | 2219 | CCCAGCAG GGAGGAAACUCC CU UCAAGGACAUCGUCCGGG UCCCAUUG | 7981 |
| 782 | UGGGAGCU G CUGGGGUG | 2220 | CACCCCAG GGAGGAAACUCC CU UCAAGGACAUCGUCCGGG AGCUCCCA | 7982 |
| 785 | GAGCUGCU G GGGUGCAG | 3180 | CUGCACCC GGAGGAAACUCC CU UCAAGGACAUCGUCCGGG AGCAGCUC | 7983 |
| 786 | AGCUGCUG G GGUGCAGG | 3181 | CCUGCACC GGAGGAAACUCC CU UCAAGGACAUCGUCCGGG CAGCAGCU | 7984 |
| 787 | GCUGCUGG G GUGCAGGA | 3182 | UCCUGCAC GGAGGAAACUCC CU UCAAGGACAUCGUCCGGG CCAGCAGC | 7985 |
| 788 | CUGCUGGG G UGCAGGAG | 2221 | CUCCUGCA GGAGGAAACUCC CU UCAAGGACAUCGUCCGGG CCCAGCAG | 7986 |
| 790 | GCUGGGGU G CAGGAGAG | 2222 | CUCUCCUG GGAGGAAACUCC CU UCAAGGACAUCGUCCGGG ACCCCAGC | 7987 |
| 793 | GGGGUGCA G GAGAGGAG | 3183 | CUCCUCUC GGAGGAAACUCC CU UCAAGGACAUCGUCCGGG UGCACCCC | 7988 |
| 794 | GGGUGCAG G AGAGGAGA | 3184 | UCUCCUCU GGAGGAAACUCC CU UCAAGGACAUCGUCCGGG CUGCACCC | 7989 |
| 796 | GUGCAGGA G AGGAGAAC | 3185 | GUUCUCCU GGAGGAAACUCC CU UCAAGGACAUCGUCCGGG UCCUGCAC | 7990 |
| 798 | GCAGGAGA G GAGAACUG | 3186 | CAGUUCUC GGAGGAAACUCC CU UCAAGGACAUCGUCCGGG UCUCCUGC | 7991 |
| 799 | CAGGAGAG G AGAACUGC | 3187 | GCAGUUCU GGAGGAAACUCC CU UCAAGGACAUCGUCCGGG CUCUCCUG | 7992 |
| 801 | GGAGAGGA G AACUGCCA | 3188 | UGGCAGUU GGAGGAAACUCC CU UCAAGGACAUCGUCCGGG UCCUCUCC | 7993 |
| 806 | GGAGAACU G CCAGAAAC | 2223 | GUUUCUGG GGAGGAAACUCC CU UCAAGGACAUCGUCCGGG AGUUCUCC | 7994 |
| 810 | AACUGCCA G AAACUGAC | 3189 | GUCAGUUU GGAGGAAACUCC CU UCAAGGACAUCGUCCGGG UGGCAGUU | 7955 |
| 816 | CAGAAACU G ACCAAAAU | 3190 | AUUUUGGU GGAGGAAACUCC CU UCAAGGACAUCGUCCGGG AGUUUCUG | 7996 |
| 830 | AAUCAUCU G UGCCCAGC | 2224 | GCUGGGCA GGAGGAAACUCC CU UCAAGGACAUCGUCCGGG AGAUGAUU | 7997 |
| 832 | UCAUCUGU G GCCAGCAG | 2225 | CUGCUGGU UGAGGAAACUCC CU UCAAGGACAUCGUCCGGG ACAGAUGA | 7998 |
| 837 | UGUGCCCA G CAUUGCUC | 2226 | GAGCACUG GGAGGAAACUCC CU UCAAGGACAUCGUCCGGG UGGGCACA | 7999 |
| 840 | GCCCAUCA G UGCUCCGG | 2227 | CCUUAGCA GGAGGAAACUCC CU UCAAGGACAUCGUCCGGG UGCUGGGC | 8000 |
| 842 | CCAGCAGU G CUCCUGUC | 2228 | GCCCGUAG GGAGGAAACUCC CU UCAAGGACAUCGUCCGGG ACUGCUGG | 8001 |
| 847 | AGUGCUCC G GGCGCUGC | 3191 | GCAGCGCC GGAGGAAACUCC CU UCAAGGACAUCUUCCGGG GGAGCACU | 8002 |
| 848 | GUGCUCCG G GCGCUGCC | 3192 | GGCAGCGC GGAGGAAACUCC CU UCAAGGACAUCGUCCGGG CGGAGCAC | 8003 |
| 849 | UGCUCCGG G GGCUUCCG | 2229 | CGGCAGCG GGAGGAAACUCC CU UCAAGGACAUCGUCCGGG CCGGAGCA | 8004 |
| 851 | CUCCGGGC G CUGCCGUG | 2230 | CACGUCAG GGAGGAAACUCC CU UCAAGGACAUCGUCCGGG GCCCGGAG | 8005 |
| 854 | CGGGCGCU G CCGUGGCA | 2231 | UGCCACGG GGAGGAAACUCC CU UCAAGGACAUCGUCCGGG AGCGCCCG | 8006 |
| 857 | GCGCUGCC G UGGCAAGU | 2232 | ACUUGCCA GGAGGAAACUCC CU UCAAGGACAUCUUCCGGG GGCAGCGC | 8007 |
| 859 | GCUGCCGU G GCAAGUCC | 3193 | GGACUUGC GGAGGAAACUCC CU UCAAGGACAUCUUCCGGG ACGGCAGC | 8008 |
| 860 | CUGCCGUG G CAAGUCCC | 2233 | GGGACUUG GGAGGAAACUCC CU UCAAGGACAUCGUCCGGG CACGGCAG | 8009 |
| 864 | CGUGGCAA G UCCCCCAG | 2234 | CUGGUGGA GGAGGAAACUCC CU UCAAGGACAUCUUCCGGG UUGCCACG | 8010 |
| 872 | GUCCCCCA G UGACUGCU | 2235 | AGCAGUCA GGAGGAAACUCC CU UCAAGGACAUCGUCCGGG UGGGGGAC | 8011 |
| 874 | CCCCCAGU G ACUUCUGC | 3194 | GCAUCAGU GGAGGAAACUCC CU UCAAGGACAUCGUCCGGG ACUGGGGG | 8012 |
| 878 | CAGUGACU G CUGCCACA | 2236 | UGUGUCAG GGAGGAAACUCC CU UCAAGGACAUCGUCCGGG AGUCACUG | 8013 |
| 881 | UGACUGCU G CCACAACC | 2237 | GGUUGUUG GGAGGAAACUCC CU UCAAGGACAUCGUCCGGG AGCAGUCA | 8014 |
| 891 | CACAACCA G UGUGCUGC | 2238 | GCAGCACA GGAGGAAACUCC CU UCAAGGACAUCGUCCGGG UGGUUGUG | 8015 |
| 893 | CAACCAGU G UGCUGCAG | 2239 | CUGCAGCA GGAGGAAACUCC CU UCAAGGACAUCGUCCGUG ACUGGUUG | 8016 |
| 895 | ACCAGUGU G GUGCAGGC | 2240 | GCCUGCAG GGAGGAAACUCC CU UCAAGUACAUCGUCCGGG ACACUGGU | 8017 |
| 898 | AGUGUUGU G GAGGCUGC | 2241 | GCAGCCUG GGAGGAAACUCC CU UCAAGUACAUCGUCCGGG AGCACACU | 8018 |
| 901 | GUGCUGCA G GCUGCACA | 3195 | UGUGCAGC GGAGGAAACUCC CU UCAAUGACAUCUUCCGGG UGCAGCAC | 8019 |
| 902 | UGCUGCAG G GUGCACAU | 2242 | CUCUGCAG GGAGGAAACUCC CU UCAAUUACAUCGUCCGGG CUGCAGCA | 8020 |
| 905 | UGCAGGCU G CACAGGCC | 2243 | GGCCUGUG GGAGGAAACUCC CU UCAAUGACAUCUUCCGGG AGCCUGCA | 8021 |
| 910 | GCUGCACA G GCCCCCGG | 3196 | CCGGGGGC GGAGGAAACUCC CU UCAAUUACAUCGUCCGGG UGUGCAGC | 8022 |
| 911 | CUGCACAG G CCCCCGGG | 2244 | CCCGGGGG GGAGGAAACUCC CU UCAAGGACAUCGUCCGGG CUGUGCAG | 8023 |
| 917 | AGGCCCCC G GGAGAGCG | 3197 | CGCUCUCC GGAGGAAACUCC CU UCAAGGACAUCGUCCGGG GGGGGCCU | 8024 |
| 918 | GGCCCCCG G GAGAGCGA | 3198 | UCGCUCUC GGAGGAAACUCC CU UCAAGGACAUCGUCCGGG CGGGGGCC | 8025 |
| 919 | GCCCCCGG G AGAGCGAC | 3199 | GUCGCUCU GGAGGAAACUCC CU UCAAGGACAUCGUCCGGG CCGGGGGC | 8026 |
| 921 | CCCCGGGA G AGCGACUG | 3200 | CAGUCGCU GGAGGAAACUCC CU UCAAGGACAUCGUCCGGG UCCCGGGG | 8027 |
| 923 | CCGGGAGA G CGACUGCC | 2245 | GGCAGUCG GGAGGAAAGUCC CU UCAAGGACAUCGUCCGGG UCUCCCGG | 8028 |
| 925 | GGGAGAGC G ACUGCCUG | 3201 | CAGGCAGU GGAGGAAACUCC CU UCAAGGACAUCGUCCGGG GCUCUCCC | 8029 |
| 929 | GAGCGACU G CCUGGUCU | 2246 | AGACCAGG GGAGGAAACUCC CU UCAAGGACAUCGUCCGGG AGUCGCUC | 8030 |
| 933 | GACUGCCU G GUCUGCCG | 3202 | CGGCAGAC GGAGGAAACUCC CU UCAAGAACAUCGUCCGGG AGGCAGUC | 8031 |
| 934 | ACUGCCCU G UCUGCCGC | 2247 | GCGGCAGA GGAGGAAACUCC CU UCAAGGACAUCGUCCGGG CAGGCAGU | 8032 |
| 938 | CCUGGUCU G CCGCAAAU | 2248 | AUUUGCGG GGAGGAAACUCC CU UCAAGGACAUCGUCCGGG AGACCAGG | 8033 |
| 941 | GGUCUGCC G CAAAUUCC | 2249 | GGAAUUUG GGAGGAAACUCC CU UCAAGGACAUCGUCCGGG GGCAGACC | 8034 |
| 950 | CAAAUUCC G AGACGAAG | 3203 | CUUCGUCU GGAGGAAACUCC CU UCAAGGACAUCGUCCGGG GGAAUUUG | 8035 |
| 952 | AAUUCCGA G ACGAAGCC | 3204 | GGCUUCGU GGAGGAAACUCC CU UCAAGGACAUCGUCCGGG UCGGAAUU | 8036 |
| 955 | UCCGACAC G AAGCCACG | 3205 | CGUCGCUU GGAGGAAACUCC CU UCAAGGACAUCGUCCGGG GUCUCGGA | 8037 |
| 958 | GAGACGAA G CCACGUGC | 2250 | GCACGUGG CGAGGAAACUCC CU UCAAGGACAUCGUCCGGG UUCGUCUC | 8038 |
| 963 | GAAGCCAC G UGCAAGGA | 2251 | UCCUUGCA GGAGGAAACUCC CU UCAAGGACAUCGUCCGGG GUGGCUUC | 8039 |
| 965 | AGCCACGU G CAAGGACA | 2252 | UGUCCUUG GGAGGAAACUCC CU UCAAGGACAUCGUCCGGG ACGUGGCU | 8040 |
| 969 | ACGUCCAA G GACACCUG | 3206 | CAGGUGUC GGAGGAAACUCC CU UCAAGGACAUCGUCCGGG UUGCACGU | 8041 |
| 970 | CGUGCAAG G ACACCUGC | 3207 | GCACGUGU GGAGGAAACUCC CU UCAAGGACAUCGUCCGGG CUUGCACG | 8042 |
| 977 | GGACACCU G CCCCCCAC | 2253 | GUGGGGGG GGAGGAAACUCC CU UCAAGGACAUCGUCCGGG AGGUGUCC | 8043 |
| 990 | CCACUCAU G CUCUACAA | 2254 | UUCUAGAG GGAGGAAACUCC CU UCAAGGACAUCGUCCGGG AUGAGUGG | 8044 |
| 1008 | CCCACCAC G UACCAGAU | 2255 | AUCUGGUA GGAGGAAACUCC CU UCAAGGACAUCGUCCGGG GUGGUGGG | 8045 |
| 1014 | ACGUACCA G AUGGAUGU | 3208 | ACAUCCAU GGAGGAAACUCC CU UCAAGGACAUCGUCCGGG UGGUACGU | 8046 |
| 1017 | UACCAGAU G GAUGUGAA | 3209 | UUCACAUC GGAGGAAACUCC CU UCAAGGACAUCGUCCGGG AUCUGGUA | 8047 |
| 1018 | ACCAGAUG G AUGUCAAC | 3210 | GUUCACAU GGAGGAAACUCC CU UCAAGGACAUCGUCCGGG CAUCUGGU | 8048 |
| 1021 | AGAUCGAU G UGAACCCC | 2256 | GGCGUUCA GGAGGAAACUCC CU UCAAGGACAUCGUCCGGG AUCCAUCU | 8049 |
| 1023 | AUGGAUCU G AACCCCGA | 3211 | UCGGGGUU GGAGGAAACUCC CU UCAAGGACAUCGUCCGGG ACAUCCAU | 8050 |
| 1030 | UGAACCCC G AGCCCAAA | 3212 | UUUGCCCU GGACGAAACUCC CU UCAAGGACAUCGUCCGGG GGGGUUCA | 8051 |
| 1032 | AACCCCGA G GGCAAAUA | 3213 | UAUUUGCC GGAGGAAACUCC CU UCAAGGACAUCGUCCGGG UCGGGGUU | 8052 |
| 1033 | ACCCCGAG G GCAAAUAC | 3214 | GUAUUUGC GGAGGAAACUCC CU UCAAGGACAUCGUCCGGG CUCGGGGU | 8053 |
| 1034 | CCCCGAGG G CAAAUACA | 2257 | UGUAUUUG GGAGGAAACUCC CU UCAAGGACAUCGUCCGGG CCUCGGGG | 8054 |
| 1043 | CAAAUACA G CUUUCCUG | 2258 | CACCAAAG GGAGGAAACUCC CU UCAAGGACAUCGUCCGGG UGUAUUUG | 8055 |
| 1048 | ACAGCUUU G GUGCCACC | 3215 | GGUGGCAC GGAGGAAACUCC CU UCAAGGACAUCGUCCGGG AAAGCUGU | 8056 |
| 1049 | CAGCUUUG G UGCCACCU | 2259 | AGGUGGCA GGAGGAAACUCC CU UCAAGGACAUCGUCCGGG CAAAGCUG | 8057 |
| 1051 | GCUUUGGU G CCACCUGC | 2260 | GCAGGUGG GGAGGAAACUCC CU UCAAGGACAUCGUCCGGG ACCAAAGC | 8058 |
| 1058 | UGCCACCU G CGUGAAGA | 2261 | UCUUCACG GGAGGAAACUCC CU UCAAGGACAUCGUCCGGG AGGUGGCA | 8069 |
| 1060 | CCACCUGC G UGAAGAAG | 2262 | CUUCUUCA GGAGGAAACUCC CU UCAAGGACAUCGUCCGGG GCAGGUGG | 8060 |
| 1062 | ACCUGCGU G AAGAAGUG | 3216 | CACUUCUU GGAGGAAACUCC CU UCAAGGACAUCGUCCGGG ACGCAGGU | 8061 |
| 1065 | UGCGUGAA G AAGUGUCC | 3217 | GGACACUU GGAGGAAACUCC CU UCAAGGACAUCGUCCGGG UUCACGCA | 8062 |
| 1068 | GUGAAGAA G UGUCCCCG | 2263 | CGGGGACA GGAGGAAACUCC CU UCAAGGACAUCGUCCGGG UUCUUCAC | 8063 |
| 1070 | GAAGAAGU G UCCCCGUA | 2264 | UACGGGGA GGAGGAAACUCC CU UCAAGGACAUCGUCCGGG ACUUCUUC | 8064 |
| 1076 | GUGUCCCC G UAAUUAUG | 2265 | CAUAAUUA GGAGGAAACUCC CU UCAAGGACAUCGUCCGGG GGGGACAC | 8065 |
| 1084 | GUAAUUAU G UGGUGACA | 2266 | UGUCACCA GGAGGAAACUCC CU UCAAGGACAUCGUCCGGG AUAAUUAC | 8066 |
| 1086 | AAUUAUGU G GUGACAGA | 3218 | UCUGUCAC GGAGGAAACUCC CU UCAAGGACAUCGUCCGGG ACAUAAUU | 8067 |
| 1087 | AUUAUGUG G UGACAGAU | 2267 | AUCUGUCA GGAGGAAACUCC CU UCAAGGACAUCGUCCGGG CACAUAAU | 8068 |
| 1089 | UAUGUGGU G ACAGAUCA | 3219 | UGAUCUGU GGAGGAAACUCC CU UCAAGGACAUCGUCCGGG ACCACAUA | 8069 |
| 1093 | UGGUGACA G AUCACGGC | 3220 | GCCGUGAU GGAGGAAACUCC CU UCAAGGACAUCGUCCGGG UGUCACCA | 8070 |
| 1099 | CAGAUCAC G GCUCGUGC | 3221 | GCACGAGC GGAGGAAACUCC CU UCAAGGACAUCGUCCGGG GUGAUCUG | 8071 |
| 1100 | AGAUCACG G CUCGUGCG | 2268 | CGCACGAG GGAGGAAACUCC CU UCAAGGACAUCGUCCGGG CGUGAUCU | 8072 |
| 1104 | CACGGCUC G UGCGUCCG | 2269 | CGGACGCA GGAGGAAACUCC CU UCAAGGACAUCGUCCGGG GAGCCGUG | 8073 |
| 1106 | CGGCUCGU G CGUCCGAG | 2270 | CUCGGACG GGAGGAAACUCC CU UCAAGGACAUCGUCCGGG ACGAGCCG | 8074 |
| 1108 | GCUCGUGC G UCCGAGCC | 2271 | GGCUCGGA GGAGGAAACUCC CU UCAAGGACAUCGUCCGGG GCACGAGC | 8075 |
| 1112 | GUGCGUCC G AGCCUGUG | 3222 | CACAGGCU GGAGGAAACUCC CU UCAAGGACAUCGUCCGGG GGACGCAC | 8076 |
| 1114 | GCGUCCGA G CCUGUGGG | 2272 | CCCACAGG GGAGGAAACUCC CU UCAAGGACAUCGUCCGGG UCGGACGC | 8077 |
| 1118 | CCGAGCCU G UGGGGCCG | 2273 | CGGCCCCA GGAGGAAACUCC CU UCAAGGACAUCGUCCGGG AGGCUCGG | 8078 |
| 1120 | GAGCCUGU G GGGCCGAC | 3223 | GUCGGCCC GGAGGAAACUCC CU UCAAGGACAUCGUCCGGG ACAGGCUC | 8079 |
| 1121 | AGCCUGUG G GGCCGACA | 3224 | UGUCGGCC GGAGGAAACUCC CU UCAAGGACAUCGUCCGGG CACAGGCU | 8080 |
| 1122 | GCCUGUGG G GCCGACAG | 3225 | CUGUCGGC GGAGGAAACUCC CU UCAAGGACAUCGUCCGGG CCACAGGC | 8081 |
| 1123 | CCUGUGGG G CCGACAGC | 2274 | GCUGUCGG GGAGGAAACUCC CU UCAAGGACAUCGUCCGGG CCCACAGG | 8082 |
| 1126 | GUGGGGCC G ACAGCUAU | 3226 | AUAGCUGU GGAGGAAACUCC CU UCAAGGACAUCGUCCGGG GGCCCCAC | 8083 |
| 1130 | GGCCGACA G CUAUGAGA | 2275 | UCUCAUAG GGAGGAAACUCC CU UCAAGGACAUCGUCCGGG UGUCGGCC | 8084 |
| 1135 | ACAGCUAU G AGAUGGAG | 3227 | CUCCAUCU GGAGGAAACUCC CU UCAAGGACAUCGUCCGGG AUAGCUGU | 8085 |
| 1137 | AGCUAUGA G AUGGAGGA | 3228 | UCCUCCAU GGAGGAAACUCC CU UCAAGGACAUCGUCCGGG UCAUAGCU | 8086 |
| 1140 | UAUGAGAU G GAGGAAGA | 3229 | UCUUCCUC GGAGGAAACUCC CU UCAAGGACAUCGUCCGGG AUCUCAUA | 8087 |
| 1141 | AUGAGAUG G AGGAAGAC | 3230 | GUCUUCCU GGAGGAAACUCC CU UCAAGGACAUCGUCCGGG CAUCUCAU | 8088 |
| 1143 | GAGAUGGA G GAAGACGG | 3231 | CCGUCUUC GGAGGAAACUCC CU UCAAGGACAUCGUCCGGG UCCAUCUC | 8089 |
| 1144 | AGAUGGAG G AAGACGGC | 3232 | GCCGUCUU GGAGGAAACUCC CU UCAAGGACAUCGUCCGGG CUCCAUCU | 8090 |
| 1147 | UGGAGGAA G ACGGCGUC | 3233 | GACGCCGU GGAGGAAACUCC CU UCAAGGACAUCGUCCGGG UUCCUCCA | 8091 |
| 1150 | AGGAAGAC G GCGUCCGC | 3234 | GCGGACGC GGAGGAAACUCC CU UCAAGGACAUCGUCCGGG GUCUUCCU | 8092 |
| 1151 | GGAAGACG G CGUCCGCA | 2276 | UGCGGACG GGAGGAAACUCC CU UCAAGGACAUCGUCCGGG CGUCUUCC | 8093 |
| 1153 | AAGACGGC G UCCGCAAG | 2277 | CUUGCGGA GGAGGAAACUCC CU UCAAGGACAUCGUCCGGG GCCGUCUU | 8094 |
| 1157 | CGGCGUCC G CAAGUGUA | 2278 | UACACUUG GGAGGAAACUCC CU UCAAGGACAUCGUCCGGG GGACGCCG | 8095 |
| 1161 | GUCCGCAA G UGUAAGAA | 2279 | UUCUUACA GGAGGAAACUCC CU UCAAGGACAUCGUCCGGG UUGCGGAC | 8096 |
| 1163 | CCGCAAGU G UAAGAAGU | 2280 | ACUUCUUA GGAGGAAACUCC CU UCAAGGACAUCGUCCGGG ACUUGCGG | 8097 |
| 1167 | AAGUGUAA G AAGUGCGA | 3235 | UCGCACUU GGAGGAAACUCC CU UCAAGGACAUCGUCCGGG UUACACUU | 8098 |
| 1170 | UGUAAGAA G UGCGAAGG | 2281 | CCUUCGCA GGAGGAAACUCC CU UCAAGGACAUCGUCCGGG UUCUUACA | 8099 |
| 1172 | UAAGAAGU G CGAAGGGC | 2282 | GCCCUUCG GGAGGAAACUCC CU UCAAGGACAUCGUCCGGG AGUUCUUA | 8100 |
| 1174 | AGAAGUGC G AAGGGCCU | 3236 | AGGCCCUU GGAGGAAACUCC CU UCAAGGACAUCGUCCGGG GCACUUCU | 8101 |
| 1177 | AGUGCGAA G GGCCUUGC | 3237 | GCAAGGCC GGAGGAAACUCC CU UCAAGGACAUCGUCCGGG UUCGCACU | 8102 |
| 1178 | GUGCGAAG G GCCUUGCC | 3238 | GGCAAGGC GGAGGAAACUCC CU UCAAGGACAUCGUCCGGG CUUGGCAC | 8103 |
| 1179 | UGCGAAGG G CCUUGCCG | 2283 | CGGCAAGG GGAGGAAACUCC CU UCAAGGACAUCGUCCGGG CCUUCGCA | 8104 |
| 1184 | AGGGCCUU G CCGCAAAG | 2284 | CUUUGCGG GGAGGAAACUCC CU UCAAGGACAUCGUCCGGG AAGGCCCU | 8105 |
| 1187 | GCCUUGCC G CAAAGUGU | 2285 | ACACUUUG GGAGGAAACUCC CU UCAAGGACAUCGUCCGGG GGCAAGGC | 8106 |
| 1192 | GCCGCAAA G UGUGUAAC | 2286 | GUUACACA GGAGGAAACUCC CU UCAAGGACAUCGUCCGGG UUUGCGGC | 8107 |
| 1194 | CGCAAAGU G UGUAACGG | 2287 | CCGUUACA GGAGGAAACUCC CU UCAAGGACAUCGUCCGGG ACUUUGCG | 8108 |
| 1196 | CAAAGUGU G UAACGGAA | 2288 | UUCCGUUA GGAGGAAACUCC CU UCAAGGACAUCGUCCGGG ACAUUUUG | 8109 |
| 1201 | UGUGUAAC G GAAUAGGU | 3239 | ACCUAUUC GGAGGAAACUCC CU UCAAGGACAUCGUCCGGG GUUACACA | 8110 |
| 1202 | GUGUAACG G AAUAGGUA | 3240 | UACCUAUU GGAGGAAACUCC CU UCAAGGACAUCGUCCGGG CGUUACAC | 8111 |
| 1207 | ACGGAAUA G GUAUUGGU | 3241 | ACCAAUAC GGAGGAAACUCC CU UCAAGGACAUCGUCCGGG UAUUCCGU | 8112 |
| 1208 | CGGAAUAG G UAUUGGUG | 2289 | CACCAAUA GGAGGAAACUCC CU UCAAGGACAUCGUCCGGG CUAUUCCG | 8113 |
| 1213 | UAGGUAUU G GUGAAUUU | 3242 | AAAUUCAC GGAGGAAACUCC CU UCAAGGACAUCGUCCGGG AAUACCUA | 8114 |
| 1214 | AGGUAUUG G UGAAUUUA | 2290 | UAAAUUCA GGAGGAAACUCC CU UCAAGGACAUCGUCCGGG CAAUACCU | 8115 |
| 1216 | GUAUUGGU G AAUUUAAA | 3243 | UUUAAAUU GGAGGAAACUCC CU UCAAGGACAUCGUCCGGG ACCAAUAC | 8116 |
| 1225 | AAUUUAAA G ACUCACUC | 3244 | GAGUGAGU GGAGGAAACUCC CU UCAAGGACAUCGUCCGGG UUUAAAUU | 8117 |
| 1243 | CAUAAAAU U CUACGAAU | 2291 | AUUCGUAG GGAGGAAACUCC CU UCAAGGACAUCGUCCGGG AUUUAUGG | 8118 |
| 1248 | AAUGCUAC G AAUAUUAA | 3245 | UUAAUAUU GGAGGAAACUCC CU UCAAGGACAUCGUCCGGG GUAGCAUU | 8119 |
| 1271 | CAAAAACU G CACCUCCA | 2292 | UGGAGGUG GGAGGAAACUCC CU UCAAGGACAUCGUCCGGG AGUUUUUG | 8120 |
| 1283 | CUCCAUCA G UGGCGAUC | 2293 | GAUCGCCA GGAGGAAACUCC CU UCAAGGACAUCGUCCGGG UGAUGGAG | 8121 |
| 1285 | CCAUCAGU G GCGAUCUC | 3246 | GAGAUCGC GGAGGAAACUCC CU UCAAGGACAUCGUCCGGG ACUGAUGG | 8122 |
| 1286 | CAUCAGUG G CGAUCUCC | 2294 | GGAGAUCG GGAGGAAACUCC CU UCAAGGACAUCGUCCGGG CACUGAUG | 8123 |
| 1288 | UCAGUGGC G AUCUCCAC | 3247 | GUGGAGAU GGAGGAAACUCC CU UCAAGGACAUCGUCCGGG GCCACUGA | 8124 |
| 1302 | CACAUCCU G CCGGUGGC | 2295 | GCCACCGG GGAGGAAACUCC CU UCAAGGACAUCGUCCGGG AGGAUGUG | 8125 |
| 1305 | AUCCUGCC G GUGGCAUU | 3248 | AAUGCCAC GGAGGAAACUCC CU UCAAGGACAUCGUCCGGG GGCAGGAU | 8126 |
| 1306 | UCCUGCCG G UGGCAUUU | 2296 | AAAUGCCA GGAGGAAACUCC CU UCAAGGACAUCGUCCGGG CGGCAGGA | 8127 |
| 1308 | CUGCCGGU G GCAUUUAG | 3249 | CUAAAUGC GGAGGAAACUCC CU UCAAGGACAUCGUCCGGG ACCGGCAG | 8128 |
| 1309 | UGCCGGUG G CAUUUAGG | 2297 | CCUAAAUG GGAGGAAACUCC CU UCAAGGACAUCGUCCGGG CACCGGCA | 8129 |
| 1316 | GGCAUUUA G GGGUGACU | 3250 | AGUCACCC GGAGGAAACUCC CU UCAAGGACAUCGUCCGGG UAAAUGCC | 8130 |
| 1317 | GCAUUUAG G GGUGACUC | 3251 | GAGUCACC GGAGGAAACUCC CU UCAAGGACAUCGUCCGGG CUAAAUGC | 8131 |
| 1318 | CAUUUAGG G GUGACUCC | 3252 | GGAGUCAC GGAGGAAACUCC CU UCAAGGACAUCGUCCGGG CCUAAAUG | 8132 |
| 1319 | AUUUAGGG G UGACUCCU | 2298 | AGGAGUCA GGAGGAAACUCC CU UCAAGGACAUCGUCCGGG CCCUAAAU | 8133 |
| 1321 | UUAGGGGU G ACUCCUUC | 3253 | GAAGGAGU GGAGGAAACUCC CU UCAAGGACAUCGUCCGGG ACCCCUAA | 8134 |
| 1347 | CCUCCUCU G GAUCCACA | 3254 | UGUGGAUC GGAGGAAACUCC CU UCAAGGACAUCGUCCGGG AGAGGAGG | 8135 |
| 1348 | CUCCUCUG G AUCCACAG | 3255 | CUGUGGAU GGAGGAAACUCC CU UCAAGGACAUCGUCCGGG CAGAGGAG | 8136 |
| 1356 | GAUCCACA G GAACUGGA | 3256 | UCCAGUUC GGAGGAAACUCC CU UCAAGGACAUCGUCCGGG UGUGGAUC | 8137 |
| 1357 | AUCCACAG G AACUGGAU | 3257 | AUCCAGUU GGAGGAAACUCC CU UCAAGGACAUCGUCCGGG CUGUGGAU | 8138 |
| 1362 | CAGGAACU G GAUAUUCU | 3258 | AGAAUAUC GGAGGAAACUCC CU UCAAGGACAUCGUCCGGG AGUUCCUG | 8139 |
| 1363 | AGGAACUG G AUAUUCUG | 3259 | CAGAAUAU GGAGGAAACUCC CU UCAAGGACAUCGUCCGGG CAGUUCCU | 8140 |
| 1371 | GAUAUUCU G AAAACCGU | 3260 | ACGGUUUU GGAGGAAACUCC CU UCAAGGACAUCGUCCGGG AGAAUAUC | 8141 |
| 1378 | UGAAAACC G UAAAGGAA | 2299 | UUCCUUUA GGAGGAAACUCC CU UCAAGGACAUCGUCCGGG GGUUUUCA | 8142 |
| 1383 | ACCGUAAA G GAAAUCAC | 3261 | GUGAUUUC GGAGGAAACUCC CU UCAAGGACAUCGUCCGGG UUUACGGU | 8143 |
| 1384 | CCGUAAAG G AAAUCACA | 3262 | UGUGAUUU GGAGGAAACUCC CU UCAAGGACAUCGUCCGGG CUUUACGG | 8144 |
| 1393 | AAAUCACA G GGUUUUUG | 3263 | CAAAAACC GGAGGAAACUCC CU UCAAGGACAUCGUCCGGG UGUGAUUU | 8145 |
| 1394 | AAUCACAG G GUUUUUGC | 3264 | GCAAAAAC GGAGGAAACUCC CU UCAAGGACAUCGUCCGGG CUGUGAUU | 8146 |
| 1395 | AUCACAGG G UUUUUGCU | 2300 | AGCAAAAA GGAGGAAACUCC CU UCAAGGACAUCGUCCGGG CCUGUGAU | 8157 |
| 1401 | GGGUUUUU G CUGAUUCA | 2301 | UGAAUCAG GGAGGAAACUCC CU UCAAGGACAUCGUCCGGG AAAAACCC | 8148 |
| 1404 | UUUUUGCU G AUUCAGGC | 3265 | GCCUGAAU GGAGGAAACUCC CU UCAAGGACAUCGUCCGGG AGCAAAAA | 8149 |
| 1410 | CUGAUUCA G GCUUGGCC | 3266 | GGCCAAGC GGAGGAAACUCC CU UCAAGGACAUCGUCCGGG UGAAUCAG | 8150 |
| 1411 | UGAUUCAG G CUUGGCCU | 2302 | AGGCCAAG GGAGGAAACUCC CU UCAAGGACAUCGUCCGGG CUGAAUCA | 8151 |
| 1415 | UCAGGCUU G GCCUGAAA | 3267 | UUUCAGGC GGAGGAAACUCC CU UCAAGGACAUCGUCCGGG AAGCCUGA | 8152 |
| 1416 | CAGGCUUG G CCUGAAAA | 2303 | UUUUCAGG GGAGGAAACUCC CU UCAAGGACAUCGUCCGGG CAAGCCUG | 8153 |
| 1420 | CUUGGCCU G AAAACAGG | 3268 | CCUGUUUU GGAGGAAACUCC CU UCAAGGACAUCGUCCGGG AGGCCAAG | 8154 |
| 1427 | UGAAAACA G GACGGACC | 3269 | GGUCCGUC GGAGGAAACUCC CU UCAAGGACAUCGUCCGGG UGUUUUCA | 8155 |
| 1428 | GAAAACAG G ACGGACCU | 3270 | AGGUCCGU GGAGGAAACUCC CU UCAAGGACAUCGUCCGGG CUGUUUUC | 8156 |
| 1431 | AACAGGAC G GACCUCCA | 3271 | UGGAGGUC GGAGGAAACUCC CU UCAAGGACAUCGUCCGGG GUCCUGUU | 8157 |
| 1432 | ACAGGACG G ACCUCCAU | 3272 | AUGGAGGU GGAGGAAACUCC CU UCAAGGACAUCGUCCGGG CGUCCUGU | 8158 |
| 1441 | ACCUCCAU G CCUUUGAG | 2304 | CUCAAAGG GGAGGAAACUCC CU UCAAGGACAUCGUCCGGG AUGGAGGU | 8159 |
| 1447 | AUGCCUUU G AGAACCUA | 3273 | UAGGUUCU GGAGGAAACUCC CU UCAAGGACAUCGUCCGGG AAAGGCAU | 8160 |
| 1449 | GCCUUUGA G AACCUAGA | 3274 | UCUAGGUU GGAGGAAACUCC CU UCAAGGACAUCGUCCGGG UCAAAGGC | 8161 |
| 1456 | AGAACCUA G AAAUCAUA | 3275 | UAUGAUUU GGAGGAAACUCC CU UCAAGGACAUCGUCCGGG UAGGUUCU | 8162 |
| 1466 | AAUCAUAC G CGGCAGGA | 2305 | UCCUGCCG GGAGGAAACUCC CU UCAAGGACAUCGUCCGGG GUAUGAUU | 8163 |
| 1468 | UCAUACGC G GCAGGACC | 3276 | GGUCCUGC GGAGGAAACUCC CU UCAAGGACAUCGUCCGGG GCGUAUGA | 8164 |
| 1469 | CAUACGCG G CAGGACCA | 2306 | UGGUCCUG GGAGGAAACUCC CU UCAAGGACAUCGUCCGGG CGCGUAUG | 8165 |
| 1472 | ACGCGGCA G GACCAAGC | 3277 | GCUUGGUC GGAGGAAACUCC CU UCAAGGACAUCGUCCGGG UGCCGCGU | 8166 |
| 1473 | GCGGGCAG G ACCAAGCA | 3278 | UGCUUGGU GGAGGAAACUCC CU UCAAGGACAUCGUCCGGG CUGCCGCG | 8167 |
| 1479 | AGGACCAA G CAACAUGG | 2307 | CCAUGUUG GGAGGAAACUCC CU UCAAGGACAUCGUCCGGG UUGGUCCU | 8168 |
| 1486 | AGCAACAU G GUCAGUUU | 3279 | AAACUGAC GGAGGAAACUCC CU UCAAGGACAUCGUCCGGG AUGUUGCU | 8169 |
| 1487 | GCAACAUG G UCAGUUUU | 2308 | AAAACUGA GGAGGAAACUCC CU UCAAGGACAUCGUCCGGG CAUGUUGC | 8170 |
| 1491 | CAUGGUCA G UUUUCUCU | 2309 | AGAGAAAA GGAGGAAACUCC CU UCAAGGACAUCGUCCGGG UGACCAUG | 8171 |
| 1501 | UUUCUCUU G CAGUCGUC | 2310 | GACGACUG GGAGGAAACUCC CU UCAAGGACAUCGUCCGGG AAGAGAAA | 8172 |
| 1504 | CUCUUGCA G UCGUCAGC | 2311 | GCUGACGA GGAGGAAACUCC CU UCAAGGACAUCGUCCGGG UGCAAGAG | 8173 |
| 1507 | UUGCAGUC G UCAGCCUG | 2312 | CAGGCUGA GGAGGAAACUCC CU UCAAGGACAUCGUCCGGG GACUGCAA | 8174 |
| 1511 | AGUCGUCA G CCUGAACA | 2313 | UGUUCAGG GGAGGAAACUCC CU UCAAGGACAUCGUCCGGG UGACGACU | 8175 |
| 1515 | GUCAGCCU G AACAUAAC | 3280 | GUUAUGUU GGAGGAAACUCC CU UCAAGGACAUCGUCCGGG AGGCUGAC | 8176 |
| 1530 | ACAUCCUU G GGAUUACG | 3281 | CGUAAUCC GGAGGAAACUCC CU UCAAGGACAUCGUCCGGG AAGGAUGU | 8177 |
| 1531 | CAUCCUUG G GAUUACGC | 3282 | GCGUAAUC GGAGGAAACUCC CU UCAAGGACAUCGUCCGGG CAAGGAUG | 8178 |
| 1532 | AUCCUUGG G AUUACGCU | 3283 | AGCGUAAU GGAGGAAACUCC CU UCAAGGACAUCGUCCGGG CCAAGGAU | 8179 |
| 1538 | GGGAUUAC G CUCCCUCA | 2314 | UGAGGGAG GGAGGAAACUCC CU UCAAGGACAUCGUCCGGG GUAAUCCC | 8180 |
| 1548 | UCCCUCAA G GAGAUAAG | 3284 | CUUAUCUC GGAGGAAACUCC CU UCAAGGACAUCGUCCGGG UUGAGGGA | 8181 |
| 1549 | CCCUCAAG G AGAUAAGU | 3285 | ACUUAUCU GGAGGAAACUCC CU UCAAGGACAUCGUCCGCG CUUCAGGG | 8182 |
| 1551 | CUCAAGGA G AUAAGUGA | 3286 | UCACUUAU GGAGGAAACUCC CU UCAAGGACAUCGUCCGGG UCCUUGAG | 8183 |
| 1556 | GGAGAUAA G UGAUGGAG | 2315 | CUCCAUCA GGAGGAAACUCC CU UCAAGGACAUCGUCCGGG UUAUCUCC | 8184 |
| 1558 | AGAUAAGU G AUGGAGAU | 3287 | AUCUCCAU GGAGGAAACUCC CU UCAAGGACAUCGUCCGGG ACUUAUCU | 8185 |
| 1561 | UAAGUGAU G GAGAUGUG | 3288 | CACAUCUC GGAGGAAACUCC CU UCAAGGACAUCGUCCGGG AUCACUUA | 8186 |
| 1562 | AAGUGAUG G AGAUGUGA | 3289 | UCACAUCU GGAGGAAACUCC CU UCAAGGACAUCGUCCGGG CAUCACUU | 8187 |
| 1564 | GUGAUGGA G AUGUGAUA | 3290 | UAUCACAU GGAGGAAACUCC CU UCAAGGACAUCGUCCGGG UCCAUCAC | 8188 |
| 1567 | AUGGAGAU G UGAUAAUU | 2316 | AAUUAUCA GGAGGAAACUCC CU UCAAGGACAUCGUCCGGG AUCUCCAU | 8189 |
| 1569 | GGAGAUGU G AUAAUUUC | 3291 | GAAAUUAU GGAGGAAACUCC CU UCAAGGACAUCGUCCGGG ACAUCUCC | 8190 |
| 1579 | UAAUUUCA G GAAACAAA | 3292 | UUUGUUUC GGAGGAAACUCC CU UCAAGGACAUCGUCCGGG UGAAAUUA | 8191 |
| 1580 | AAUUUCAG G AAACAAAA | 3293 | UUUUGUUU GGAGGAAACUCC CU UCAAGGACAUCGUCCGGG CUGAAAUU | 8192 |
| 1593 | AAAAAUUU G UGCUAUGC | 2317 | GCAUAGCA GGAGGAAACUCC CU UCAAGGACAUCGUCCGGG AAAUUUUU | 8193 |
| 1595 | AAAUUUGU G CUAUGCAA | 2318 | UUGCAUAG GGAGGAAACUCC CU UCAAGGACAUCGUCCGGG ACAAAUUU | 8194 |
| 1600 | UGUGCUAU G CAAAUACA | 2319 | UGUAUUUG GGAGGAAACUCC CU UCAAGGACAUCGUCCGGG AUAGCACA | 8195 |
| 1616 | AAUAAACU G GAAAAAAC | 3294 | GUUUUUUC GGAGGAAACUCC CU UCAAGGACAUCGUCCGGG AGUUUAUU | 8196 |
| 1617 | AUAAACUG G AAAAAACU | 3295 | AGUUUUUU GGAGGAAACUCC CU UCAAGGACAUCGUCCGGG CAGUUUAU | 8197 |
| 1626 | AAAAAACU G UUUGGGAC | 2320 | GUCCCAAA GGAGGAAACUCC CU UCAAGGACAUCGUCCGGG AGUUUUUU | 8198 |
| 1630 | AACUGUUU G GGACCUCC | 3296 | GGAGGUCC GGAGGAAACUCC CU UCAAGGACAUCGUCCGGG AAACAGUU | 8199 |
| 1631 | ACUGUUUG G GACCUCCG | 3297 | CGGAGGUC GGAGGAAACUCC CU UCAAGGACAUCGUCCGGG CAAACAGU | 8200 |
| 1632 | CUGUUUGG G ACCUCCGG | 3298 | CCGGAGGU GGAGGAAACUCC CU UCAAGGACAUCGUCCGGG CCAAACAG | 8201 |
| 1639 | GGACCUCC G GUCAGAAA | 3299 | UUUCUGAC GGAGGAAACUCC CU UCAAGGACAUCGUCCGGG GGAGGUCC | 8202 |
| 1640 | GACCUCCG G UCAGAAAA | 2321 | UUUUCUGA GGAGGAAACUCC CU UCAAGGACAUCGUCCGGG CGGAGGUC | 8203 |
| 1644 | UCCGGUCA G AAAACCAA | 3300 | UUGGUUUU GGAGGAAACUCC CU UCAAGGACAUCGUCCGGG UGACCGGA | 8204 |
| 1661 | AAUUAUAA G CAACAGAG | 2322 | CUCUGUUG GGAGGAAACUCC CU UCAAGGACAUCGUCCGGG UUAUAAUU | 8205 |
| 1667 | AAGCAACA G AGGUGAAA | 3301 | UUUCACCU GGAGGAAACUCC CU UCAAGGACAUCGUCCGGG UGUUGCUU | 8206 |
| 1669 | GCAACAGA G GUGAAAAC | 3302 | GUUUUCAC GGAGGAAACUCC CU UCAAGGACAUCGUCCGGG UCUGUUGC | 8207 |
| 1670 | CAACAGAG G UGAAAACA | 2323 | UGUUUUCA GGAGGAAACUCC CU UCAAGGACAUCGUCCGGG CUCUGUUG | 8208 |
| 1672 | ACAGAGGU G AAAACAGC | 3303 | GCUGUUUU GGAGGAAACUCC CU UCAAGGACAUCGUCCGGG ACCUCUGU | 8209 |
| 1679 | UGAAAACA G CUGCAAGG | 2324 | CCUUGCAG GGAGGAAACUCC CU UCAAGGACAUCGUCCGGG UGUUUUCA | 8210 |
| 1682 | AAACAGCU G CAAGGCCA | 2325 | UGGCCUUG GGAGGAAACUCC CU UCAAGGACAUCGUCCGGG AGCUGUUU | 8211 |
| 1686 | AGCUGCAA G GCCACAGG | 3304 | CCUGUGGC GGAGGAAACUCC CU UCAAGGACAUCGUCCGGG UUGCAGCU | 8212 |
| 1687 | GCUGCAAG G CCACAGGC | 2326 | GCCUGUGG GGAGGAAACUCC CU UCAAGGACAUCGUCCGGG CUUGCAGC | 8213 |
| 1693 | AGGCCACA G GCCAGGUC | 3305 | GACCUGGC GGAGGAAACUCC CU UCAAGGACAUCGUCCGGG UGUGGCCU | 8214 |
| 1694 | GGCCACAG G CCAGGUCU | 2327 | AGACCUGG GGAGGAAACUCC CU UCAAGGACAUCGUCCGGG CUGUGGCC | 8215 |
| 1698 | ACAGGCCA G GUCUGCCA | 3306 | UGGCAGAC GGAGGAAACUCC CU UCAAGGACAUCGUCCGGG UGGCCUGU | 8216 |
| 1699 | CAGGCCAG G UCUGCCAU | 2328 | AUGGCAGA GGAGGAAACUCC CU UCAAGGACAUCGUCCGGG CUGGCCUG | 8217 |
| 1703 | CCAGGUCU G CCAUGCCU | 2329 | AGGCAUGG GGAGGAAACUCC CU UCAAGGACAUCGUCCGGG AGACCUGG | 8218 |
| 1708 | UCUGCCAU G CCUUGUGC | 2330 | GCACAAGG GGAGGAAACUCC CU UCAAGGACAUCGUCCGGG AUGGCAGA | 8219 |
| 1713 | CAUGCCUU G UGCUCCCC | 2331 | GGGGAGCA GGAGGAAACUCC CU UCAAGGACAUCGUCCGGG AAGGCAUG | 8220 |
| 1715 | UGCCUUGU G CUCCCCCG | 2332 | CGGGGGAG GGAGGAAACUCC CU UCAAGGACAUCGUCCGGG ACAAGGCA | 8221 |
| 1723 | GCUCCCCC G AGGGCUGC | 3307 | GCAGCCCU GGAGGAAACUCC CU UCAAGGACAUCGUCCGGG GGGGGAGC | 8222 |
| 1725 | UCCCCCGA G GGCUGCUG | 3308 | CAGCAGCC GGAGGAAACUCC CU UCAAGGACAUCGUCCGGG UCGGGGGA | 8223 |
| 1726 | CCCCCGAG G GCUGCUGG | 3309 | CCAGCAGC GGAGGAAACUCC CU UCAAGGACAUCUUCCGGG CUCGGGGG | 8224 |
| 1727 | CCCCGAGG G GUUCUGUG | 2333 | CCCAGCAG GGAGGAAACUCC CU UCAAGGACAUCGUCCGGG CCUCGGGG | 8225 |
| 1730 | CGAGGGCU G CUGGGUCC | 2334 | GGCCCCAG GGAGGAAACUCC CU UCAAGGACAUCGUCCGGG AGCCCUCG | 8226 |
| 1733 | GGGCUGCU G GGGCCCGG | 3310 | CCGGGCCC GGAGGAAACUCC CU UCAAGGACAUCGUCCGGG AGCAGCCC | 8227 |
| 1734 | GGCUGCUG G GGCCCGGA | 3311 | UCCGGGCC GGAGGAAACUCC CU UCAAGGACAUCGUCCGUG CAGCAGCC | 8228 |
| 1735 | GCUGCUGG G GCCCGGAG | 3312 | CUCCGGGC GGAGGAAACUCC CU UCAAGGACAUCGUCCGUG CCAGCAGC | 8229 |
| 1736 | CUGCUUGG G CCCGGAGC | 2335 | GCUCCGGG GGAGGAAACUCC CU UCAAGGACAUCGUCCGGG CCCAGCAG | 8230 |
| 1740 | UGGGGCCC G GAGCCCAG | 3313 | CUGGGCUC GGAGGAAACUCC CU UCAAGGACAUCGUCCGGG GGGCCCCA | 8231 |
| 1741 | GGGGCCCG G AGCCCAGG | 3314 | CCUGGGCU GGAGGAAACUCC CU UCAAGGACAUCGUCCGGG CGGGCCCC | 8232 |
| 1743 | GGCCCGGA G CCCAGGGA | 2336 | UCCCUGGG GGAGGAAACUCC CU UCAAGGACAUCGUCCGGG UCCGGGCC | 8233 |
| 1748 | GGAGCCCA G GGACUGCG | 3315 | CGCAGUCC GGAGGAAACUCC CU UCAAGGACAUCGUCCGGG UGGGCUCC | 8234 |
| 1749 | GAUCCCAG G GACUGCGU | 3316 | ACGCAGUC GGAGGAAACUCC CU UCAAGGACAUCGUCCGGG CUGGGCUC | 8235 |
| 1750 | AGCCCAGG G ACUGCGUC | 3317 | GACGCAGU GGAGGAAACUCC CU UCAAGGACAUCGUCCGGG CCUGGGCU | 8236 |
| 1754 | CAGGGACU G CGUCUCUU | 2337 | AAGAGACG GGAGGAAACUCC CU UCAAGGACAUCGUCCGGG AGUCCCUG | 8237 |
| 1756 | GGGACUGC G UCUCUUGC | 2338 | GCAAGAGA GGAGGAAACUCC CU UCAAGGACAUCGUCCGGG GCAGUCCC | 8238 |
| 1763 | CGUCUCUU G CCGGAAUG | 2339 | CAUUCCGG GGAGGAAACUCC CU UCAAGGACAUCGUCCGGG AAGAGACG | 8239 |
| 1766 | CUCUUGCC G GAAUGUCA | 3318 | UGACAUUC GGAGGAAACUCC CU UCAAGGACAUCGUCCGGG GGCAAGAG | 8240 |
| 1767 | UCUUGCCG G AAUGUCAG | 3319 | CUGACAUU GGAGGAAACUCC CU UCAAGGACAUCGUCCGGG CGGCAAGA | 8241 |
| 1771 | GCCGGAAU G UCAGCCGA | 2340 | UCGGCUGA GGAGGAAACUCC CU UCAAGGACAUCGUCCGGG AUUCCGGC | 8242 |
| 1775 | GAAUGUCA G CCGAGGCA | 2341 | UGCCUCGG GGAGGAAACUCC CU UCAAGGACAUCGUCCGGG UGACAUUC | 8243 |
| 1778 | UGUCAGCC G AGGCAGGG | 3320 | CCCUGCCU GGAGGAAACUCC CU UCAAGGACAUCCUCCGGG GGCUGACA | 8244 |
| 1780 | UCAGCCGA G GCAGGGAA | 3321 | UUCCCUGC GGAGGAAACUCC CU UCAAGGACAUCCUCCGGG UCGGCUGA | 8245 |
| 1781 | CAGCCGAG G CAGGGAAU | 2342 | AUUCCCUG GGAGGAAACUCC CU UCAAGGACAUCCUCCGGG CUCGGCUG | 8246 |
| 1784 | CCGAGGCA G GGAAUGCG | 3322 | CGCAUUCC GGAGGAAACUCC CU UCAAGGACAUCCUCCGGG UGCCUCGG | 8247 |
| 1785 | CGAGGCAG G GAAUGCGU | 3323 | ACGCAUUC GGAGGAAACUCC CU UCAAGGACAUCCUCCGGG CUGCCUCG | 8248 |
| 1786 | GAGGCAGG G AAUGCGUG | 3324 | CACGCAUU GGAGGAAACUCC CU UCAAGGACAUCCUCCGGG CCUGCCUC | 8249 |
| 1790 | GAGGGAAU G CGUGGACA | 2343 | UGUCCACG GGAGGAAACUCC CU UCAAGGACAUCCUCCGGG AUUCCCUG | 8250 |
| 1792 | GGGAAUGC G UGGACAAG | 2344 | CUUGUCCA GGAGGAAACUCC CU UCAAGGACAUCCUCCGGG GCAUUCCC | 8251 |
| 1794 | GAAUGCGU G GACAAGUG | 3325 | CACUUGUC GGAGGAAACUCC CU UCAAGGACAUCCUCCGGG ACGCAUUC | 8252 |
| 1795 | AAUGCGUG G ACAAGUGC | 3326 | GCACUUGU GGAGGAAACUCC CU UCAAGGACAUCCUCCGGG CACGCAUU | 8253 |
| 1800 | GUGGACAA G UGCAAGCU | 2345 | AGCUUGCA GGAGGAAACUCC CU UCAAGGACAUCCUCCGGG UUGUCCAC | 8254 |
| 1802 | GGACAAGU G CAAGCUUC | 2346 | GAAGCUUG GGAGGAAACUCC CU UCAAGGACAUCCUCCGGG ACUUGUCC | 8255 |
| 1806 | AAGUGCAA G CUUCUGGA | 2347 | UCCAGAAG GGAGGAAACUCC CU UCAAGGACAUCCUCCGGG UUGCACUU | 8256 |
| 1812 | AAGCUUCU G GAGGGUGA | 3327 | UCACCCUC GGAGGAAACUCC CU UCAAGGACAUCCUCCGGG AGAAGCUU | 8257 |
| 1813 | AGCUUCUG G AGGGUGAG | 3328 | CUCACCCU GGAGGAAACUCC CU UCAAGGACAUCCUCCGGG CAGAAGCU | 8258 |
| 1815 | CUUCUGGA G GGUGAGCC | 3329 | GGCUCACC GGAGGAAACUCC CU UCAAGGACAUCCUCCGGG UCCAGAAG | 8259 |
| 1816 | UUCUGGAG G GUGAGCCA | 3330 | UGGCUCAC GGAGGAAACUCC CU UCAAGGACAUCCUCCGGG CUCCAGAA | 8260 |
| 1817 | UCUGGAGG G UGAGCCAA | 2348 | UUGGCUCA GGAGGAAACUCC CU UCAAGGACAUCCUCCGGG CCUCCAGA | 8261 |
| 1819 | UGGAGGGU G AGCCAAGG | 3331 | CCUUGGCU GGAGGAAACUCC CU UCAAGGACAUCCUCCGGG ACCCUCCA | 8262 |
| 1821 | GAGGGUGA G CCAAGGGA | 2349 | UCCCUUGG GGAGGAAACUCC CU UCAAGGACAUCCUCCGGG UCACCCUC | 8263 |
| 1826 | UGAGCCAA G GGAGUUUG | 3332 | CAAACUCC GGAGGAAACUCC CU UCAAGGACAUCCUCCGGG UUGGCUCA | 8264 |
| 1827 | GAGCCAAG G GAGUUUGU | 3333 | ACAAACUC GGAGGAAACUCC CU UCAAGGACAUCCUCCGGG CUUGGCUC | 8265 |
| 1828 | AGCCAAGG G AGUUUGUG | 3334 | CACAAACU GGAGGAAACUCC CU UCAAGGACAUCGUCCGGG CCUUGGCU | 8266 |
| 1830 | CCAAGGGA G UUUGUGGA | 2350 | UCCACAAA GGAGGAAACUCC CU UCAAGGACAUCGUCCGGG UCCCUUGG | 8267 |
| 1834 | GGGAGUUU G UGGAGAAC | 2351 | GUUCUCCA GGAGGAAACUCC CU UCAAGGACAUCGUCCGGG AAACUCCC | 8268 |
| 1836 | GAGUUUGU G GAGAACUC | 3335 | GAGUUCUC GGAGGAAACUCC CU UCAAGGACAUCGUCCGGG ACAAACUC | 8269 |
| 1837 | AGUUUGUG G AGAACUCU | 3336 | AGAGUUCU GGAGGAAACUCC CU UCAAGGACAUCGUCCGGG CACAAACU | 8270 |
| 1839 | UUUGUGGA G AACUCUGA | 3337 | UCAGAGUU GGAGGAAACUCC CU UCAAGGACAUCGUCCGGG UCCACAAA | 8271 |
| 1846 | AGAACUCU G AGUGCAUA | 3338 | UAUGCACU GGAGGAAACUCC CU UCAAGGACAUCGUCCGGG AGAGUUCU | 8272 |
| 1848 | AACUCUGA G UGCAUACA | 2352 | UGUAUGCA GGAGGAAACUCC CU UCAAGGACAUGGUCCGGG UCAGAGUU | 8273 |
| 1850 | CUCUGAGU G CAUACAGU | 2353 | ACUGUAUG GGAGGAAACUCC CU UCAAGGACAUCGUCCGGG ACUCAGAG | 8274 |
| 1857 | UGCAUACA G UGCCACCC | 2354 | GGGUGGCA GGAGGAAACUCC CU UCAAGGACAUCGUCCGGG UGUAUGCA | 8275 |
| 1859 | CAUACAGU G CCACCCAG | 2355 | CUGGGUGG GGAGGAAACUCC CU UCAAGGACAUCGUCCGGG ACUGUAUG | 8276 |
| 1867 | GCCACCCA G AGUGCCUG | 3339 | CAGGCACU GGAGGAAACUCC CU UCAAGGACAUCGUCCGGG UGGGUGGC | 8277 |
| 1869 | CACCCAGA G UGCCUGCC | 2356 | GGCAGGCA GGAGGAAACUCC CU UCAAGGACAUCGUCCGGG UCUGGGUG | 8278 |
| 1871 | CCCAGAGU G CCUGCCUC | 2357 | GAGGCAGG GGAGGAAACUCC CU UCAAGGACAUCGUCCGGG ACUCUGGG | 8279 |
| 1875 | GAGUGCCU G CCUCAGGC | 2358 | GCCUGAGG GGAGGAAACUCC CU UCAAGGACAUCGUCCGGG AGGCACUC | 8280 |
| 1881 | CUGCCUCA G GCCAUGAA | 3340 | UUCAUGGC GGAGGAAACUCC CU UCAAGGACAUCGUCCGGG UGAGGCAG | 8281 |
| 1882 | UGCCUCAG G CCAUGAAC | 2359 | GUUCAUGG GGAGGAAACUCC CU UCAAGGACAUCGUCCGGG CUGAGGCA | 8282 |
| 1887 | CAGGCCAU G AACAUCAC | 3341 | GUGAUGUU GGAGGAAACUCC CU UCAAGGACAUCGUCCGGG AUGGCCUG | 8283 |
| 1898 | CAUCACCU G CACAGGAC | 2360 | GUCCUGUG GGAGGAAACUCC CU UCAAGGACAUCGUCCGGG AGGUGAUG | 8284 |
| 1903 | CCUGCACA G GACGGGGA | 3342 | UCCCCGUC GGAGGAAACUCC CU UCAAGGACAUCGUCCGGG UGUGCAGG | 8285 |
| 1904 | CUGCACAG G ACGGGGAC | 3343 | GUCCCCGU GGAGGAAACUCC CU UCAAGGACAUCGUCCGGG CUGUGCAG | 8286 |
| 1907 | CACAGGAC G GGGACCAG | 3344 | CUGGUCCC GGAGGAAACUCC CU UCAAGGACAUCGUCCGGG GUCCUGUG | 8287 |
| 1908 | ACAGGACG G GGACCAGA | 3345 | UCUGGUCC GGAGGAAACUCC CU UCAAGGACAUCGUCCGGG CGUCCUGU | 8288 |
| 1909 | CAGGACGG G GACCAGAC | 3346 | GUCUGGUC GGAGGAAACUCC CU UCAAGGACAUCGUCCGGG CCGUCCUG | 8289 |
| 1910 | AGGACGGG G ACCAGACA | 3347 | UGUCUGGU GGAGGAAACUCC CU UCAAGGACAUCGUCCGGG CCCGUCCU | 8290 |
| 1915 | GGGGACCA G ACAACUGU | 3348 | ACAGUUGU GGAGGAAACUCC CU UCAAGGACAUCGUCCGGG UGGUCCCC | 8291 |
| 1922 | AGACAACU G UAUCCAGU | 2361 | ACUGGAUA GGAGGAAACUCC CU UCAAGGACAUCGUCCGGG AGUUGUCU | 8292 |
| 1929 | UGUAUCCA G UGUGCCCA | 2362 | UGGGCACA GGAGGAAACUCC CU UCAAGGACAUCGUCCGGG UGGAUACA | 8293 |
| 1931 | UAUCCAGU G UGCCCACU | 2363 | AGUGGGCA GGAGGAAACUCC CU UCAAGGACAUCGUCCGGG ACUGGAUA | 8294 |
| 1933 | UCCAGUGU G CCCACUAC | 2364 | GUAGUGGG GGAGGAAACUCC CU UCAAGGACAUCGUCCGGG ACACUGGA | 8295 |
| 1945 | ACUACAUU G ACGGCCCC | 3349 | GGGGCCGU GGAGGAAACUCC CU UCAAGGACAUCGUCCGGG AAUGUAGU | 8296 |
| 1948 | ACAUUGAC G GCCCCCAC | 3350 | GUGGGGGC GGAGGAAACUCC CU UCAAGGACAUCGUCCGGG GUCAAUGU | 8297 |
| 1949 | CAUUGACG G CCCCCACU | 2365 | AGUGGGGG GGAGGAAACUCC CU UCAAGGACAUCGUCCGGG CGUCAAUG | 8298 |
| 1958 | CCCCCACU G CGUCAAGA | 2366 | UCUUGACG GGAGGAAACUCC CU UCAAGGACAUCGUCCGGG AGUGGGGG | 8299 |
| 1960 | CCCACUGC G UCAAGACC | 2367 | GGUCUUGA GGAGGAAACUCC CU UCAAGGACAUCGUCCGGG GCAGUGGG | 8300 |
| 1965 | UGCGUCAA G ACCUGCCC | 3351 | GGGCAGGU GGAGGAAACUCC CU UCAAGGACAUCGUCCGGG UUGACGCA | 8301 |
| 1970 | CAAGACCU G CCCGGCAG | 2368 | CUGCCGGG GGAGGAAACUCC CU UCAAGGACAUCGUCCGGG AGGUCUUG | 8302 |
| 1974 | ACCUGCCC G GCAGGAGU | 3352 | ACUCCUGC GGAGGAAACUCC CU UCAAGGACAUCGUCCGGG GGGCAGGU | 8303 |
| 1975 | CCUGCCCG G CAGGAGUC | 2369 | GACUCCUG GGAGGAAACUCC CU UCAAGGACAUCGUCCGGG CGGGCAGG | 8304 |
| 1978 | GCCCGGCA G GAGUCAUG | 3353 | CAUGACUC GGAGGAAACUCC CU UCAAGGACAUCGUCCGGG UGCCGGGC | 8305 |
| 1979 | CCCGGCAG G AGUCAUGG | 3354 | CCAUGACU GGAGGAAACUCC CU UCAAGGACAUCGUCCGGG CUGCCGGG | 8306 |
| 1981 | CGGCAGGA G UCAUGGGA | 2370 | UCCCAUGA GGAGGAAACUCC CU UCAAGGACAUCGUCCGGG UCCUGCCG | 8307 |
| 1986 | GGAGUCAU G GGAGAAAA | 3355 | UUUUCUCC GGAGGAAACUCC CU UCAAGGACAUCGUCCGGG AUGACUCC | 8308 |
| 1987 | GAGUCAUG G GAGAAAAC | 3356 | GUUUUCUC GGAGGAAACUCC CU UCAAGGACAUCGUCCGGG CAUGACUC | 8309 |
| 1988 | AGUCAUGG G AGAAAACA | 3357 | UGUUUUCU GGAGGAAACUCC CU UCAAGGACAUCGUCCGGG CCAUGACU | 8310 |
| 1990 | UCAUGGGA G AAAACAAC | 3358 | GUUGUUUU GGAGGAAACUCC CU UCAAGGACAUCGUCCGGG UCCCAUGA | 8311 |
| 2004 | AACACCCU G GUCUGGAA | 3359 | UUCCAGAC GGAGGAAACUCC CU UCAAGGACAUCGUCCGGG AGGGUGUU | 8312 |
| 2005 | ACACCCUG G UCUGGAAG | 2371 | CUUCCAGA GGAGGAAACUCC CU UCAAGGACAUCGUCCGGG CAGGGUGU | 8313 |
| 2009 | CCUGGUCU G GAAGUACG | 3360 | CGUACUUC GGAGGAAACUCC CU UCAAGGACAUCGUCCGGG AGACCAGG | 8314 |
| 2010 | CUGGUCUG G AAGUACGC | 3361 | GCGUACUU GGAGGAAACUCC CU UCAAGGACAUCGUCCGGG CAGACCAG | 8315 |
| 2013 | GUCUGGAA G UACGCAGA | 2372 | UCUGCGUA GGAGGAAACUCC CU UCAAGGACAUCGUCCGGG UUCCAGAC | 8316 |
| 2017 | GGAAGUAC G CAGACGCC | 2373 | GGCGUCUG GGAGGAAACUCC CU UCAAGGACAUCGUCCGGG GUACUUCC | 8317 |
| 2020 | AGUACGCA G ACGCCGGC | 3362 | GCCGGCGU GGAGGAAACUCC CU UCAAGGACAUCGUCCGGG UGCGUACU | 8318 |
| 2023 | ACGCAGAC G CCGGCCAU | 2374 | AUGGCCGG GGAGGAAACUCC CU UCAAGGACAUCGUCCGGG GUCUGCGU | 8319 |
| 2026 | CAGACGCC G GCCAUGUG | 3353 | CACAUGGC GGAGGAAACUCC CU UCAAGGACAUCGUCCGGG GGCGUCUG | 8320 |
| 2027 | AGACGCCG G CCAUGUGU | 2375 | ACACAUGG GGAGGAAACUCC CU UCAAGGACAUCGUCCGGG CGGCGUCU | 8321 |
| 2032 | CCGGCCAU G UGUGCCAC | 2376 | GUGGCACA GGAGGAAACUCC CU UCAAGGACAUCGUCCGGG AUGGCCGG | 8322 |
| 2034 | GGCCAUGU G UGCCACCU | 2377 | AGGUGGCA GGAGGAAACUCC CU UCAAGGACAUCGUCCGGG ACAUGGCC | 8323 |
| 2036 | CCAUGUGU G CCACCUGU | 2378 | ACAGGUGG GGAGGAAACUCC CU UCAAGGACAUCGUCCGGG ACACAUGG | 8324 |
| 2043 | UGCCACCU G UGCCAUCC | 2379 | GGAUGGCA GGAGGAAACUCC CU UCAAGGACAUCGUCCGGG AGGUGGCA | 8325 |
| 2045 | CCACCUGU G CCAUCCAA | 2380 | UUGGAUGG GGAGGAAACUCC CU UCAAGGACAUCGUCCGGG ACAGGUGG | 8326 |
| 2057 | UCCAAACU G CACCUACG | 2381 | CGUAGGUG GGAGGAAACUCC CU UCAAGGACAUCGUCCGGG AGUUUGGA | 8327 |
| 2065 | GCACCUAC G GAUGCACU | 3364 | AGUGCAUC GGAGGAAACUCC CU UCAAGGACAUCGUCCGGG GUAGGUGC | 8328 |
| 2066 | CACCUACG G AUGCACUG | 3365 | CAGUGCAU GGAGGAAACUCC CU UCAAGGACAUCGUCCGGG CGUAGGUG | 8329 |
| 2069 | CUACGGAU G CACUGGGC | 2382 | GCCCAGUG GGAGGAAACUCC CU UCAAGGACAUCGUCCGGG AUCCGUAG | 8330 |
| 2074 | GAUGCACU G GGCCAGGU | 3366 | ACCUGGCC GGAGGAAACUCC CU UCAAGGACAUCGUCCGGG AGUGCAUC | 8331 |
| 2075 | AUGCACUG G GCCAGGUC | 3367 | GACCUGGC GGAGGAAACUCC CU UCAAGGACAUCGUCCGGG CAGUGCAU | 8332 |
| 2076 | UGCACUGG G CCAGGUCU | 2383 | AGACCUGG GGAGGAAACUCC CU UCAAGGACAUCGUCCGGG CCAGUGCA | 8333 |
| 2080 | CUGGGCCA G GUCUUGAA | 3368 | UUCAAGAC GGAGGAAACUCC CU UCAAGGACAUCGUCCGGG UGGCCCAG | 8334 |
| 2081 | UGGGCCAG G UCUUGAAG | 2384 | CUUCAAGA GGAGGAAACUCC CU UCAAGGACAUCGUCCGGG CUGGCCCA | 8335 |
| 2086 | CAGGUCUU G AAGGCUGU | 3369 | ACAGCCUU GGAGGAAACUCC CU UCAAGGACAUCGUCCGGG AAGACCUG | 8336 |
| 2089 | GUCUUGAA G GCUGUCCA | 3370 | UGGACAGC GGAGGAAACUCC CU UCAAGGACAUCGUCCGGG UUCAAGAC | 8337 |
| 2090 | UCUUGAAG G CUGUCCAA | 2385 | UUGGACAG GGAGGAAACUCC CU UCAAGGACAUCGUCCGGG CUUCAAGA | 8338 |
| 2093 | UGAAGGCU G UCCAACGA | 2386 | UCGUUGGA GGAGGAAACUCC CU UCAAGGACAUCGUCCGGG AGCCUUCA | 8339 |
| 2100 | UGUCCAAC G AAUGGGCC | 3371 | GGCCCAUU GGAGGAAACUCC CU UCAAGGACAUCGUCCGGG GUUGGACA | 8340 |
| 2104 | CAACGAAU G GGCCUAAG | 3372 | CUUAGGCC GGAGGAAACUCC CU UCAAGGACAUCGUCCGGG AUUCGUUG | 8341 |
| 2105 | AACGAAUG G GCCUAAGA | 3373 | UCUUAGGC GGAGGAAACUCC CU UCAAGGACAUCGUCCGGG CAUUCUGG | 8342 |
| 2106 | ACGAAUGG G CCUAAGAU | 2387 | AUCUUAGG GGAGGAAACUCC CU UCAAGGACAUCGUCCGGG CCAUUCGU | 8343 |
| 2112 | GGGCCUAA G AUCCCGUC | 3374 | GACGGGAU GGAGGAAACUCC CU UCAAGGACAUCGUCCGGG UUAGGCCC | 8344 |
| 2118 | AAGAUCCC G UCCAUCGC | 2388 | GCGAUGGA GGAGGAAACUCC CU UCAAGGACAUCGUCCGGG GGGAUCUU | 8345 |
| 2125 | CGUCCAUC G CCACUGGG | 2389 | CCCAGUGG GGAGGAAACUCC CU UCAAGGACAUCGUCCGGG GAUGGACG | 8346 |
| 2131 | UCGCCACU G GGAUGGUG | 3375 | CACCAUCC GGAGGAAACUCC CU UCAAGGACAUCGUCCGGG AGUGGCGA | 8347 |
| 2132 | CGCCACUG G GAUGGUGG | 3376 | CCACCAUC GGAGGAAACUCC CU UCAAGGACAUCGUCCGGG CAGUGGCG | 8348 |
| 2133 | GCCACUGG G AUGGUGGG | 3377 | CCCACCAU GGAGGAAACUCC CU UCAAGGACAUCGUCCGGG CCAGUGGC | 8349 |
| 2136 | ACUGGGAU G GUGGGGGC | 3378 | GCCCCCAC GGAGGAAACUCC CU UCAAGGACAUCGUCCGGG AUCCCAGU | 8350 |
| 2137 | CUGGGAUG G UGGGGGCC | 2390 | GGCCCCCA GGAGGAAACUCC CU UCAAGGACAUCGUCCGGG CAUCCCAG | 8351 |
| 2139 | GGGAUGGU G GGGGCCCU | 3379 | AGGGCCCC GGAGGAAACUCC CU UCAAGGACAUCGUCCGGG ACCAUCCC | 8352 |
| 2140 | GGAUGGUG G GGGCCCUC | 3380 | GAGGGCCC GGAGGAAACUCC CU UCAAGGACAUCGUCCGGG CACCAUCC | 8353 |
| 2141 | GAUGGUGG G GGCCCUCC | 3381 | GGAGGGCC GGAGGAAACUCC CU UCAAGGACAUCGUCCGGG CCACCAUC | 8354 |
| 2142 | AUGGUGGG G GCCCUCCU | 3382 | AGGAGGGC GGAGGAAACUCC CU UCAAGGACAUCGUCCGGG CCCACCAU | 8355 |
| 2143 | UGGUGGGG G CCCUCCUC | 2388 | GAGGAGGG GGAGGAAACUCC CU UCAAGGACAUCGUCCGGG CCCCACCA | 8356 |
| 2154 | CUCCUCUU G CUGCUGGU | 2389 | ACCAGCAG GGAGGAAACUCC CU UCAAGGACAUCGUCCGGG AAGAGGAC | 8357 |
| 2157 | CUCUUGCU G CUGGUGGU | 2393 | ACCACCAG GGAGGAAACUCC CU UCAAGGACAUCGUCCGGG AGCAAGAG | 8358 |
| 2160 | UUGCUGCU G GUGGUGGC | 3383 | GCCACCAC GGAGGAAACUCC CU UCAAGGACAUCGUCCGGG AGCAGCAA | 8359 |
| 2161 | UGCUGCUG G UGGUGGCC | 3394 | AGGGCCAC GGAGGAAACUCC CU UCAAGGACAUCGUCCGGG ACCAGCAG | 8361 |
| 2163 | CUGCUGGU G GUGGCCCU | 3384 | GCCCCCAC GGAGGAAACUCC CU UCAAGGACAUCGUCCGGG AUCCCAGU | 8350 |
| 2164 | UGCUGGUG G UGGCCCUG | 2395 | CAGGGCCA GGAGGAAACUCC CU UCAAGGACAUCGUCCGGG CACCAGCA | 8362 |
| 2166 | CUGGUGGU G GCCCUGGG | 3385 | CCCAGGGC GGAGGAAACUCC CU UCAAGGACAUCGUCCGGG ACCACCAG | 8363 |
| 2167 | UGGUGGUG G CCCUGGGG | 2396 | CCCCAGGG GGAGGAAACUCC CU UCAAGGACAUCGUCCGGG CACCACCA | 8364 |
| 2172 | GUGGCCCU G GGGAUCGG | 3386 | CCGAUCCC GGAGGAAACUCC CU UCAAGGACAUCGUCCGGG AGGGCCAC | 8365 |
| 2173 | UGGCCCUG G GGAUCGGC | 3387 | GCCGAUCC GGAGGAAACUCC CU UCAAGGACAUCGUCCGGG CAGGGCCA | 8366 |
| 2174 | GGCCCUGG G GAUCGGCC | 3388 | GGCCGAUC GGAGGAAACUCC CU UCAAGGACAUCGUCCGGG CCAGGGCC | 8367 |
| 2175 | GCCCUGGG G AUCGGCCU | 3389 | AGGCCGAU GGAGGAAACUCC CU UCAAGGACAUCGUCCGGG CCCAGGGC | 8368 |
| 2179 | UGGGGAUC G GCCUCUUC | 3390 | GAAGAGGC GGAGGAAACUCC CU UCAAGGACAUCGUCCGGG GAUCCCCA | 8369 |
| 2180 | GGGGAUCG G CCUCUUCA | 2397 | UGAAGAGG GGAGGAAACUCC CU UCAAGGACAUCGUCCGGG CGAUCCCC | 8370 |
| 2190 | CUCUUCAU G CGAAGGCG | 2398 | CGCCUUCG GGAGGAAACUCC CU UCAAGGACAUCGUCCGGG AUGAAGAG | 8371 |
| 2192 | CUUCAUGC G AAGGCGCC | 3391 | GGCGCCUU GGAGGAAACUCC CU UCAAGGACAUCGUCCGGG GCAUGAAG | 8372 |
| 2195 | CAUGCGAA G GCGCCACA | 3392 | UGUGGCGC GGAGGAAACUCC CU UCAAGGACAUCGUCCGGG UUCGCAUG | 8373 |
| 2196 | AUGCGAAG G CGCCACAU | 2399 | AUGUGGCG GGAGGAAACUCC CU UCAAGGACAUCGUCCGGG CUUCGCAU | 8374 |
| 2198 | GCGAAGGC G CCACAUCG | 2400 | CGAUGUGG GGAGGAAACUCC CU UCAAGGACAUCGUCCGGG GCCUUCGC | 8375 |
| 2206 | GCCACAUC G UUCGGAAG | 2401 | CUUCCGAA GGAGGAAACUCC CU UCAAGGACAUCGUCCGGG GAUGUGGC | 8376 |
| 2210 | CAUCGUUC G GAAGCGCA | 3393 | UGCGCUUC GGAGGAAACUCC CU UCAAGGACAUCGUCCGGG GAACGAUG | 8377 |
| 2211 | AUCGUUCG G AAGCGCAC | 3394 | GUGCGCUU GGAGGAAACUCC CU UCAAGGACAUCGUCCGGG CGAACGAU | 8378 |
| 2214 | GUUCGGAA G CGCACGCU | 2402 | AGCGUGCG GGAGGAAACUCC CU UCAAGGACAUCGUCCGGG UUCCGAAC | 8379 |
| 2216 | UCGGAAGC G CACGCUGC | 2403 | GCAGCGUG GGAGGAAACUCC CU UCAAGGACAUCGUCCGGG GCUUCCGA | 8380 |
| 2220 | AAGCGCAC G CUGCGGAG | 2404 | CUCCGCAG GGAGGAAACUCC CU UCAAGGACAUCGUCCGGG GUGCGCUU | 8381 |
| 2223 | CGCACGCU G CGGAGGCU | 2405 | AGCCUCCG GGAGGAAACUCC CU UCAAGGACAUCGUCCGGG AGCGUGCG | 8382 |
| 2225 | CACGCUGC G GAGGCUGC | 3395 | GCAGCCUC GGAGGAAACUCC CU UCAAGGACAUCGUCCGGG GCAGCGUG | 8383 |
| 2226 | ACGCUGCG G AGGCUGCU | 3396 | AGCAGCCU GGAGGAAACUCC CU UCAAGGACAUCGUCCGGG CGCAGCGU | 8384 |
| 2228 | GCUGCGGA G GCUGCUGC | 3397 | GCAGCAGC GGAGGAAACUCC CU UCAAGGACAUCGUCCGGG UCCGCAGC | 8385 |
| 2229 | CUGCGGAG G CUGCUGCA | 2406 | UGCAGCAG GGAGGAAACUCC CU UCAAGGACAUCGUCCGGG CUCCGCAG | 8386 |
| 2232 | CGGAGGCU G CUGCAGGA | 2407 | UCCUGCAG GGAGGAAACUCC CU UCAAGGACAUCGUCCGGG AGCCUCCG | 8387 |
| 2235 | AGGCUGCU G CAGGAGAG | 2408 | CUCUCCUG GGAGGAAACUCC CU UCAAGGACAUCGUCCGGG AGCAGCCU | 8388 |
| 2238 | CUGCUGCA G GAGAGGGA | 3398 | UCCCUCUC GGAGGAAACUCC CU UCAAGGACAUCGUCCGGG UGCAGCAG | 8389 |
| 2239 | UGCUGCAG G AGAGGGAG | 3399 | CUCCCUCU GGAGGAAACUCC CU UCAAGGACAUCGUCCGGG CUGCAGCA | 8390 |
| 2241 | CUGCAGGA G AGGGAGCU | 3400 | AGCUCCCU GGAGGAAACUCC CU UCAAGGACAUCGUCCGGG UCCUGCAG | 8391 |
| 2243 | GCAGGAGA G GGAGCUUG | 3401 | CAAGCUCC GGAGGAAACUCC CU UCAAGGACAUCGUCCGGG UCUCCUGC | 8392 |
| 2244 | CAGGAGAG G GAGCUUGU | 3402 | ACAAGCUC GGAGGAAACUCC CU UCAAGGACAUCGUCCGGG CYCYCCUG | 8393 |
| 2245 | AGGAGAGG G AGCUUGUG | 3403 | CACAAGCU GGAGGAAACUCC CU UCAAGGACAUCGUCCGGG CCUCUCCU | 8394 |
| 2247 | GAGAGGGA G CUUGUGGA | 2409 | UCCACAAG GGAGGAAACUCC CU UCAAGGACAUCGUCCGGG UCCCUCUC | 8395 |
| 2251 | GGGAGCUU G UGGAGCCU | 2410 | AGGCUCCA GGAGGAAACUCC CU UCAAGGACAUCGUCCGGG AAGCUCCC | 8396 |
| 2253 | GAGCUUGU G GAGCCUCU | 3404 | AGAGGCUC GGAGGAAACUCC CU UCAAGGACAUCGUCCGGG ACAAGCUC | 8397 |
| 2254 | AGCUUGUG G AGCCUCUU | 3405 | AAGAGGCU GGAGGAAACUCC CU UCAAGGACAUCGUCCGGG CACAAGCU | 8398 |
| 2256 | CUUGUGGA G CCUCUUAC | 2411 | GUAACAGG GGAGGAAACUCC CU UCAAGGACAUCGUCCGGG UCCACAAG | 8399 |
| 2270 | UACACCCA G UGGAGAAG | 2412 | CUUCUCCA GGAGGAAACUCC CU UCAAGGACAUCGUCCGGG UGGGUGUA | 8400 |
| 2272 | CACCCAGU G GAGAAGCU | 3406 | AGCUUCUC GGAGGAAACUCC CU UCAAGGACAUCGUCCGGG ACUGGGUG | 8401 |
| 2273 | ACCCAGUG G AGAAGCUC | 3407 | GAGCUUCU GGAGGAAACUCC CU UCAAGGACAUCGUCCGGG CACUGGGU | 8402 |
| 2275 | CCAGUGGA G AAGCUCCC | 3408 | GGGAGCUU GGAGGAAACUCC CU UCAAGGACAUCGUCCGGG UCCACUGG | 8403 |
| 2278 | GUGGAGAA G CUCCCAAC | 2413 | GUUGGGAG GGAGGAAACUCC CU UCAAGGACAUCGUCCGGG UUCUCCAC | 8404 |
| 2290 | CCAACCAA G CUCUCUUG | 2414 | CAAGAGAG GGAGGAAACUCC CU UCAAGGACAUCGUCCGGG UUGGUUGG | 8405 |
| 2298 | GCUCUCUU G AGGAUCUU | 3409 | AAGAUCCU GGAGGAAACUCC CU UCAAGGACAUCGUCCGGG AAGAGAGC | 8406 |
| 2300 | UCUCUUGA G GAUCUUGA | 3410 | UCAAGAUC GGAGGAAACUCC CU UCAAGGACAUCGUCCGGG UCAAGAGA | 8407 |
| 2301 | CUCUUGAG G AUCUUGAA | 3411 | UUCAAGAU GGAGGAAACUCC CU UCAAGGACAUCGUCCGGG CUCAAGAG | 8408 |
| 2307 | AGGAUCUU G AAGGAAAC | 3412 | GUUUCCUU GGACGAAACUCC CU UCAAGGACAUCGUCCGGG AAGAUCCU | 8409 |
| 2310 | AUCUUGAA G GAAACUGA | 3413 | UCAGUUUC GGAGGAAACUCC CU UCAAGGACAUCGUCCGGG UUCAAGAU | 8410 |
| 2311 | UCUUGAAG G AAACUGAA | 3414 | UUCAGUUU GGAGGAAACUCC CU UCAAGGACAUCGUCCGGG CUUCAAGA | 8411 |
| 2317 | AGGAAACU G AAUUCAAA | 3415 | UUUGAAUU GGAGGAAACUCC CU UCAAGGACAUCGUCCGGG AGUUUCCU | 8412 |
| 2328 | UUCAAAAA G AUCAAAGU | 3416 | ACUUUGAU GGAGGAAACUCC CU UCAAGGACAUCGUCCGGG UUUUUGAA | 8413 |
| 2335 | AGAUCAAA G UGCUGGGC | 2415 | GCCCAGCA GGAGGAAACUCC CU UCAAGGACAUCGUCCGGG UUUGAUCU | 8414 |
| 2337 | AUCAAAGU G CUGGGCUC | 2416 | GAGCCCAG GGAGGAAACUCC CU UCAAGGACAUCGUCCGGG ACUUUGAU | 8415 |
| 2340 | AAAGUGCU G GGCUCCGG | 3417 | CCGGAGCC GGAGGAAACUCC CU UCAAGGACAUCGUCCGGG AGCACUUU | 8416 |
| 2341 | AAGUGCUG G GCUCCGGU | 3418 | ACCGGAGC GGAGGAAACUCC CU UCAAGGACAUCGUCCGGG CAGCACUU | 8417 |
| 2342 | AGUGCUGG G CUCCGGUG | 2417 | CACCGGAG GGAGGAAACUCC CU UCAAGGACAUCGUCCGGG CCAGCACU | 8418 |
| 2347 | UGGGCUCC G GUGCGUUC | 3419 | GAACGCAC GGAGGAAACUCC CU UCAAGGACAUCGUCCGGG GGAGCCCA | 8419 |
| 2348 | GGGCUCCG G UGCGUUCG | 2418 | CGAACGCA GGAGGAAACUCC CU UCAAGGACAUCGUCCGGG CGGAGCCC | 8420 |
| 2350 | GCUCCGGU G CGUUCGGC | 2419 | GCCGAACG GGAGGAAACUCC CU UCAAGGACAUCGUCCGGG ACCGGAGC | 8421 |
| 2352 | UCCGGUGC G UUCGGCAC | 2420 | GUGCCGAA GGAGGAAACUCC CU UCAAGGACAUCGUCCGGG GCACCGGA | 8422 |
| 2356 | GUGCGUUC G GCACGGUG | 3420 | CACCGUGC GGAGGAAACUCC CU UCAAGGACAUCGUCCGGG GAACGCAC | 8423 |
| 2357 | UGCGUUCG G CACGGUGU | 2421 | ACACCGUG GGAGGAAACUCC CU UCAAGGACAUCGUCCGGG CGAACGCA | 8424 |
| 2361 | UUCGGCAC G GUGUAUAA | 3421 | UUAUACAC GGAGGAAACUCC CU UCAAGGACAUCGUCCGGG GUGCCGAA | 8425 |
| 2362 | UCGGCACG G UGUAUAAG | 2422 | CUUAUACA GGAGGAAACUCC CU UCAAGGACAUCGUCCGGG CGUGCCGA | 8426 |
| 2364 | GGCACGGU G UAUAAGGG | 2423 | CCCUUAUA GGAGGAAACUCC CU UCAAGGACAUCGUCCGGG ACCGUGCC | 8427 |
| 2370 | GUGUAUAA G GGACUCUG | 3422 | CAGAGUCC GGAGGAAACUCC CU UCAAGGACAUCGUCCGGG UUAUACAC | 8428 |
| 2371 | UGUAUAAG G GACUCUGG | 3423 | CCAGAGUC GGAGGAAACUCC CU UCAAGGACAUCGUCCGGG CUUAUACA | 8429 |
| 2372 | GUAUAAGG G ACUCUGGA | 3424 | UCCAGAGU GGAGGAAACUCC CU UCAAGGACAUCGUCCGGG CCUUAUAC | 8430 |
| 2378 | GGGACUCU G GAUCCCAG | 3425 | CUGGGAUC GGAGGAAACUCC CU UCAAGGACAUCGUCCGGG AGAGUCCC | 8431 |
| 2379 | GGACUCUG G AUCCCAGA | 3426 | UCUGGGAU GGAGGAAACUCC CU UCAAGGACAUCGUCCGGG CAGAGUCC | 8432 |
| 2386 | GGAUCCCA G AAGGUGAG | 3427 | CUCACCUU GGAGGAAACUCC CU UCAAGGACAUCGUCCGGG UGGGAUCC | 8433 |
| 2389 | UCCCAGAA G GUGAGAAA | 3428 | UUUCUCAC GGAGGAAACUCC CU UCAAGGACAUCGUCCGGG UUCUGGGA | 8434 |
| 2390 | CCCAGAAG G UGAGAAAG | 2424 | CUUUCUCA GGAGGAAACUCC CU UCAAGGACAUCGUCCGGG CUUCUGGG | 8435 |
| 2392 | CAGAAGGU G AGAAAGUU | 3429 | AACUUUCU GGAGGAAACUCC CU UCAAGGACAUCGUCCGGG ACCUUCUG | 8436 |
| 2394 | GAAGGUGA G AAAGUUAA | 3430 | UUAACUUU GGAGGAAACUCC CU UCAAGGACAUCGUCCGGG UCACCUUC | 8437 |
| 2398 | GUGAGAAA G UUAPAAUU | 2425 | AAUUUUAA GGAGGAAACUCC CU UCAAGGACAUCGUCCGGG UUUCUCAC | 8438 |
| 2410 | AAAUUCCC G UCGCUAUC | 2426 | GAUAGCGA GGAGGAAACUCC CU UCAAGGACAUCGUCCGGG GGGAAUUU | 8439 |
| 2413 | UUCCCGUC G CUAUCAAG | 2427 | CUUGAUAG GGAGGAAACUCC CU UCAAGGACAUCGUCCGGG GACGGGAA | 8440 |
| 2421 | GCUAUCAA G GAAUUAAG | 3431 | CUUAAUUC GGAGGAAACUCC CU UCAAGGACAUCGUCCGGG UUGAUAGC | 8441 |
| 2422 | CUAUCAAG G AAUUAAGA | 3432 | UCUUAAUU GGAGGAAACUCC CU UCAAGGACAUCGUCCGGG CUUGAUAG | 8442 |
| 2429 | GGAAUUAA G AGAAGCAA | 3433 | UUGCUUCU GGAGGAAACUCC CU UCAAGGACAUCGUCCGGG UUAAUUCC | 8443 |
| 2431 | AAUUAAGA G AAGCAACA | 3434 | UGUUGCUU GGAGGAAACUCC CU UCAAGGACAUCGUCCGGG UCUUAAUU | 8444 |
| 2434 | UAAGAGAA G CAACAUCU | 2428 | AGAUGUUG GGAGGAAACUCC CU UCAAGGACAUCGUCCGGG UUCUCUUA | 8445 |
| 2445 | ACAUCUCC G AAAGCCAA | 3435 | UUGGCUUU GGAGGAAACUCC CU UCAAGGACAUCGUCCGGG GGAGAUGU | 8446 |
| 2449 | CUCCGAAA G CCAACAAG | 2429 | CUUGUUGG GGAGGAAACUCC CU UCAAGGACAUCGUCCGGG UUUCGGAG | 8447 |
| 2457 | GCCAACAA G GAAAUCCU | 3436 | AGGAUUUC GGAGGAAACUCC CU UCAAGGACAUCGUCCGGG UUGUUGGC | 8448 |
| 2458 | CCAACAAG G AAAUCCUC | 3437 | GAGGAUUU GGAGGAAACUCC CU UCAAGGACAUCGUCCGGG CUUGUUGG | 8449 |
| 2467 | AAAUCCUC G AUGAAGCC | 3438 | GGCUUCAU GGAGGAAACUCC CU UCAAGGACAUCGUCCGGG GAGGAUUU | 8450 |
| 2470 | UCCUCGAU G AAGCCUAC | 3439 | GUAGGCUU GGAGGAAACUCC CU UCAAGGACAUCGUCCGGG AUCGAGGA | 8451 |
| 2473 | UCGAUGAA G CCUACGUG | 2430 | CACGUAGG GGAGGAAACUCC CU UCAAGGACAUCGUCCGGG UUCAUCGA | 8452 |
| 2479 | AAGCCUAC G UGAUGGCC | 2431 | GGCCAUCA GGAGGAAACUCC CU UCAAGGACAUCGUCCGGG GUAGGCUU | 8453 |
| 2481 | GCCUACGU G AUGGCCAG | 3440 | CUGGCCAU GGAGGAAACUCC CU UCAAGGACAUCGUCCGGG ACGUAGGC | 8454 |
| 2484 | UACGUGAU G GCCAGCGU | 3441 | ACGCUGGC GGAGGAAACUCC CU UCAAGGACAUCGUCCGGG AUCACGUA | 8455 |
| 2485 | ACGUGAUG G CCAGCGUG | 2432 | CACGCUGG GGAGGAAACUCC CU UCAAGGACAUCGUCCGGG CAUCACGU | 8456 |
| 2489 | GAUGGCCA G CGUGGACA | 2433 | UGUCCACG GGAGGAAACUCC CU UCAAGGACAUCGUCCGGG UGGCCAUC | 8457 |
| 2491 | UGGCCAGC G UGGACAAC | 2434 | GUUGUCCA GGAGGAAACUCC CU UCAAGGACAUCGUCCGGG GCUGGCCA | 8458 |
| 2493 | GCCAGCGU G GACAACCC | 3442 | GGGUUGUC GGAGGAAACUCC CU UCAAGGACAUCGUCCGGG ACGCUGGC | 8459 |
| 2494 | CCAGCGUG G ACAACCCC | 3443 | GGGGUUGU GGAGGAAACUCC CU UCAAGGACAUCGUCCGGG CACGCUGG | 8460 |
| 2506 | ACCCCCAC G UGUGCCGC | 2435 | GCGGCACA GGAGGAAACUCC CU UCAAGGACAUCGUCCGGG GUGGGGGU | 8461 |
| 2508 | CCCCACGU G UGCCGCCU | 2436 | AGGCGGCA GGAGGAAACUCC CU UCAAGGACAUCGUCCGGG ACGUGGGG | 8462 |
| 2510 | CCACGUGU G CCGCCUGC | 2437 | GCAGGCGG GGAGGAAACUCC CU UCAAGGACAUCGUCCGGG ACACGUGG | 8463 |
| 2513 | CGUGUGCC G CCUGCUGG | 2438 | CCAGCAGG GGAGGAAACUCC CU UCAAGGACAUCGUCCGGG GGCACACG | 8464 |
| 2517 | UGCCGCCU G CUGGGCAU | 2439 | AUGCCCAG GGAGGAAACUCC CU UCAAGGACAUCGUCCGGG AGGCGGCA | 8465 |
| 2520 | CGCCUGCU G GGCAUCUG | 3444 | CAGAUGCC GGAGGAAACUCC CU UCAAGGACAUCGUCCGGG AGCAGGCG | 8466 |
| 2521 | GCCUGCUG G UCAUCUGC | 3445 | GCAGAUGC GGAGGAAACUCC CU UCAAGGACAUCGUCCGGG CAGCAGGC | 8467 |
| 2522 | CCUGCUGG G CAUCUGCC | 2440 | GGCAGAUG GGAGGAAACUCC CU UCAAGGACAUCGUCCGGG CCAGCAGG | 8458 |
| 2528 | GGGCAUCU G CCUCACCU | 2441 | AGGUGAGG GGAGGAAACUCC CU UCAAGGACAUCGUCCGGG AGAUGCCC | 8469 |
| 2542 | CCUCCACC G UGCAACUC | 2442 | GAGUUGCA GGAGGAAACUCC CU UCAAGGACAUCGUCCGGG GGUGGAGG | 8470 |
| 2544 | UCCACCGU G CAACUCAU | 2443 | AUGAGUUG GGAGGAAACUCC CU UCAAGGACAUCGUCCGGG ACGGUGGA | 8471 |
| 2556 | CUCAUCAC G CAGCUCAU | 2444 | AUGAGCUG GGAGGAAACUCC CU UCAAGGACAUCGUCCGGG GUGAUGAG | 8472 |
| 2559 | AUCACGCA G CUCAUGCC | 2435 | GGCAUGAG GGAGGAAACUCC CU UCAAGGACAUCGUCCGGG UGCGUGAU | 8473 |
| 2565 | CAGCUCAU G CCCUUCGG | 2446 | CCGAAGGG GGAGGAAACUCC CU UCAAGGACAUCGUCCGGG AUGAGCUG | 8474 |
| 2572 | UGCCCUUC G GCUGCCUC | 3446 | GAGGCAGC GGAGGAAACUCC CU UCAAGGACAUCGUCCGGG GAAGGGCA | 8475 |
| 2573 | GCCCUUCG G CUGCCUCC | 2447 | GGAGGCAG GGAGGAAACUCC CU UCAAGGACAUCGUCCGGG CGAAGGGC | 8476 |
| 2576 | CUUCGGCU G CCUCCUGG | 2448 | CCAGGAGG GGAGGAAACUCC CU UCAAGGACAUCGUCCGGG AGCCGAAG | 8477 |
| 2583 | UGCCUCCU G GACUAUGU | 3447 | ACAUAGUC GGAGGAAACUCC CU UCAAGGACAUCGUCCGGG AGGAGGCA | 8478 |
| 2584 | GCCUCCUG G ACUAUGUC | 3448 | GACAUAGU GGAGGAAACUCC CU UCAAGGACAUCGUCCGGG CAGGAGGC | 8479 |
| 2590 | UGGACUAU G UCCGGGAA | 2449 | UUCCCGGA GGAGGAAACUCC CU UCAAGGACAUCGUCCGGG AUAGUCCA | 8480 |
| 2594 | CUAUGUCC G GGAACACA | 3449 | UGUGUUCC GGAGGAAACUCC CU UCAAGGACAUCGUCCGGG GGACAUAG | 8481 |
| 2595 | UAUGUCCG G GAACACAA | 3450 | UUGUGUUC GGAGGAAACUCC CU UCAAGGACAUCGUCCGGG CGGACAUA | 8482 |
| 2596 | AUGUCCGG G AACACAAA | 3451 | UUUGUGUU GGAGGAAACUCC CU UCAAGGACAUCGUCCGGG CCGGACAU | 8483 |
| 2605 | AACACAAA G ACAAUAUU | 3452 | AAUAUUGU GGAGGAAACUCC CU UCAAGGACAUCGUCCGGG UUUGUGUU | 8484 |
| 2614 | ACAAUAUU G GCUCCCAG | 3453 | CUGGGAGC GGAGGAAACUCC CU UCAAGGACAUCGUCCGGG AAUAUUGU | 8885 |
| 2615 | CAAUAUUG G CUCCCAGU | 2450 | ACUGGGAG GGAGGAAACUCC CU UCAAGGACAUCGUCCGGG CAAUAUUG | 8486 |
| 2622 | GGCUCCCA G UACCUGCU | 2451 | AGCAGGUA GGAGGAAACUCC CU UCAAGGACAUCGUCCGGG UGGGAGCC | 8487 |
| 2628 | CAGUACCU G CUCAACUG | 2452 | CAGUUGAG GGAGGAAACUCC CU UCAAGGACAUCGUCCGGG AGGUACUG | 8488 |
| 2636 | GCUCAACU G GUGUGUGC | 3454 | GCACACAC GGAGGAAACUCC CU UCAAGGACAUCGUCCGGG AGUUGAGC | 8489 |
| 2637 | CUCAACUG G UGUGUGCA | 2453 | UGCACACA GGAGGAAACUCC CU UCAAGGACAUCGUCCGGG CAGUUGAG | 8490 |
| 2639 | CAACUGGU G UGUGCAGA | 2454 | UCUGCACA GGAGGAAACUCC CU UCAAGGACAUCGUCCGGG ACCAGUUG | 8491 |
| 2641 | ACUGGUGU G UGCAGAUC | 2455 | GAUCUGCA GGAGGAAACUCC CU UCAAGGACAUCGUCCGGG ACACCAGU | 8492 |
| 2643 | UGGUGUGU G CAGAUCGC | 2456 | GCGAUCUG GGAGGAAACUCC CU UCAAGGACAUCGUCCGGG ACACACCA | 8493 |
| 2646 | UGUGUGCA G AUCGCAAA | 3455 | UUUGCGAU GGAGGAAACUCC CU UCAAGGACAUCGUCCGGG UGCACACA | 8494 |
| 2650 | UGCAGAUC G CAAAGGGC | 2457 | GCCCUUUG GGAGGAAACUCC CU UCAAGGACAUCGUCCGGG GAUCUGCA | 8495 |
| 2655 | AUCGCAAA G GGCAUGAA | 3456 | UUCAUGCC GGAGGAAACUCC CU UCAAGGACAUCGUCCGGG UUUGCGAU | 8496 |
| 2656 | UCGCAAAG G GCAUGAAC | 3457 | GUUCAUGC GGAGGAAACUCC CU UCAAGGACAUCGUCCGGG CUUUGCGA | 8497 |
| 2657 | CGCAAAGG G CAUGAACU | 2458 | AGUUCAUG GGAGGAAACUCC CU UCAAGGACAUCGUCCGGG CCUUUGCG | 8498 |
| 2661 | AAGGGCAU G AACUACUU | 3458 | AAGUAGUU GGAGGAAACUCC CU UCAAGGACAUCGUCCGGG AUGCCCUU | 8499 |
| 2670 | AACUACUU G GAGGACCG | 3459 | CGGUCCUC GGAGGAAACUCC CU UCAAGGACAUCGUCCGGG AAGUAGUU | 8500 |
| 2671 | ACUACUUG G AGGACCGU | 3460 | ACGGUCCU GGAGGAAACUCC CU UCAAGGACAUCGUCCGGG CAAGUAGU | 8501 |
| 2673 | UACUUGGA G GACCGUCG | 3461 | CGACGGUC GGAGGAAACUCC CU UCAAGGACAUCGUCCGGG UCCAAGUA | 8502 |
| 2674 | ACUUGGAG G ACCGUCGC | 3462 | GCGACGGU GGAGGAAACUCC CU UCAAGGACAUCGUCCGGG CUCCAAGU | 8503 |
| 2678 | GGAGGACC G UCGCUUGG | 2459 | CCAAGCGA GGAGGAAACUCC CU UCAAGGACAUCGUCCGGG GGUCCUCC | 8504 |
| 2681 | GGACCGUC G CUUGGUGC | 2460 | GCACCAAG GGAGGAAACUCC CU UCAAGGACAUCGUCCGGG GACGGUCC | 8505 |
| 2685 | CGUCGCUU G GUGCACCG | 2463 | CGGUGCAC GGAGGAAACUCC CU UCAAGGACAUCGUCCGGG AAGCGACG | 8506 |
| 2686 | GUCGCUUG G UGCACCGC | 3461 | GCGGUGCA GGAGGAAACUCC CU UCAAGGACAUCGUCCGGG CAAGCGAC | 8507 |
| 2688 | CGCUUGGU G CACCGCCA | 2462 | UCGCGGUG GGAGGAAACUCC CU UCAAGGACAUCGUCCGGG ACCAAGCG | 8508 |
| 2693 | GGUGCACC G CGACCUGG | 3463 | CGACCUGG GGAGGAAACUCC CU UCAAGGACAUCGUCCGGG GGUGCACC | 8509 |
| 2695 | UGCACCGC G ACCUGGCA | 3464 | GUUCAUGC GGAGGAAACUCC CU UCAAGGACAUCGUCCGGG CUUUGCGA | 8497 |
| 2700 | CGCGACCU G GCAGCCAG | 3465 | CUGGCUGC GGAGGAAACUCC CU UCAAGGACAUCGUCCGGG AGGUCGCG | 8511 |
| 2701 | GCGACCUG G CAGCCAGG | 2464 | CCUGGCUG GGAGGAAACUCC CU UCAAGGACAUCGUCCGGG CAGGUCGC | 8512 |
| 2704 | ACCUGGCA G CCAGGAAC | 2465 | GUUCCUGG GGAGGAAACUCC CU UCAAGGACAUCGUCCGGG UGCCAGGU | 8513 |
| 2708 | GGCAGCCA G GAACGUAC | 3466 | GUACGUUC GGAGGAAACUCC CU UCAAGGACAUCGUCCGGG UGGCUGCC | 8514 |
| 2709 | GCAGCCAG G AACGUACU | 3467 | AGUACGUU GGAGGAAACUCC CU UCAAGGACAUCGUCCGGG CUGGCUGC | 8515 |
| 2713 | CCAGGAAC G UACUGGUG | 2466 | CACCAGUA GGAGGAAACUCC CU UCAAGGACAUCGUCCGGG CUUCCUGG | 8516 |
| 2718 | AACGUACU G GUGAAAAC | 3468 | GUUUUCAC GGAGGAAACUCC CU UCAAGGACAUCGUCCGGG AGUACGUU | 8517 |
| 2719 | ACGUACUG G UGAAAACA | 2467 | UGUUUUCA GGAGGAAACUCC CU UCAAGGACAUCGUCCGGG CAGUACGU | 8518 |
| 2721 | GUACUGGU G AAAACACC | 3469 | GGUGUUUU GGAGGAAACUCC CU UCAAGGACAUCGUCCGGG ACCAGUAC | 8519 |
| 2730 | AAAACACC G CAGCAUGU | 2468 | ACAUGCUG GGAGGAAACUCC CU UCAAGGACAUCGUCCGGG GGUGUUUU | 8520 |
| 2733 | ACACCGCA G CAUGUCAA | 2469 | UUGACAUG GGAGGAAACUCC CU UCAAGGACAUCGUCCGGG UGCGGUGU | 8521 |
| 2737 | CGCAGCAU G UCAAGAUC | 2470 | GAUCUUGA GGAGGAAACUCC CU UCAAGGACAUCGUCCGGG AUGCUGCG | 8522 |
| 2742 | CAUGUCAA G AUCACAGA | 3470 | UCUGUGAU GGAGGAAACUCC CU UCAAGGACAUCGUCCGGG UUGACAUG | 8523 |
| 2749 | AGAUCACA G AUUUUGGG | 3471 | CCCAAAAU GGAGGAAACUCC CU UCAAGGACAUCGUCCGGG UGUGAUCU | 8524 |
| 2755 | CAGAUUUU G GGCUGGCC | 3472 | GGCCAGCC GGAGGAAACUCC CU UCAAGGACAUCGUCCGGG AAAAUCUG | 8525 |
| 2756 | AGAUUUUG G GCUGGCCA | 3473 | UGGCCAGC GGAGGAAACUCC CU UCAAGGACAUCGUCCGGG CAAAAUCU | 8526 |
| 2757 | GAUUUUGG G CUGGCCAA | 2471 | UUGGCCAG GGAGGAAACUCC CU UCAAGGACAUCGUCCGGG CCAAAAUC | 8527 |
| 2760 | UUUGGGCU G GCCAAACU | 3474 | AGUUUGGC GGAGGAAACUCC CU UCAAGGACAUCGUCCGGG AGCCCAAA | 8528 |
| 2761 | UUGGGCUG G CCAAACUG | 2472 | CAGUUUGG GGAGGAAACUCC CU UCAAGGACAUCGUCCGGG CAGCCCAA | 8529 |
| 2769 | GCCAAACU G CUGGGUGC | 2473 | GCACCCAG GGAGGAAACUCC CU UCAAGGACAUCGUCCGGG AGUUUGGC | 8530 |
| 2772 | AAACUGCU G GGUGCGGA | 3475 | UCCGCACC GGAGGAAACUCC CU UCAAGGACAUCGUCCGGG AGCAGUUU | 8531 |
| 2773 | AACUGCUG G GUGCGGAA | 3476 | UUCCGCAC GGAGGAAACUCC CU UCAAGGACAUCGUCCGGG CAGCAGUU | 8532 |
| 2774 | ACUGCUGG G UGCGGAAG | 2474 | CUUCCGCA GGAGGAAACUCC CU UCAAGGACAUCGUCCGGG CCAGCAGU | 8533 |
| 2776 | UGCUGGGU G CGGAAGAG | 2475 | CUCUUCCG GGAGGAAACUCC CU UCAAGGACAUCGUCCGGG ACCCAGCA | 8534 |
| 2778 | CUGGGUGC G GAAGAGAA | 3477 | UUCUCUUC GGAGGAAACUCC CU UCAAGGACAUCGUCCGGG GCACCCAG | 8535 |
| 2779 | UGGGUGCG G AAGAGAAA | 3478 | UUUCUCUU GGAGGAAACUCC CU UCAAGGACAUCGUCCGGG CGCACCCA | 8536 |
| 2782 | GUGCGGAA G AGAAAGAA | 3479 | UUCUUUCU GGAGGAAACUCC CU UCAAGGACAUCGUCCGGG UUCCGCAC | 8537 |
| 2784 | GCGGAAGA G AAAGAAUA | 3480 | UAUUCUUU GGAGGAAACUCC CU UCAAGGACAUCGUCCGGG UCUUCCGC | 8538 |
| 2788 | AAGAGAAA G AAUACCAU | 3481 | AUGGUAUU GGAGGAAACUCC CU UCAAGGACAUCGUCCGGG UUUCUCUU | 8539 |
| 2797 | AAUACCAU G CAGAAGGA | 2476 | UCCUUCUG GGAGGAAACUCC CU UCAAGGACAUCGUCCGGG AUGGUAUU | 8540 |
| 2800 | ACCAUGCA G AAGGAGGC | 3482 | GCCUCCUU GGAGGAAACUCC CU UCAAGGACAUCGUCCGGG UGCAUGGU | 8541 |
| 2803 | AUGCAGAA G GAGGCAAA | 3483 | UUUGCCUC GGAGGAAACUCC CU UCAAGGACAUCGUCCGGG UUCUGCAU | 8542 |
| 2804 | UGCAGAAG G AGGCAAAG | 3484 | CUUUGCCU GGAGGAAACUCC CU UCAAGGACAUCGUCCGGG CUUCUGCA | 8543 |
| 2806 | CAGAAGGA G GCAAAGUG | 3485 | CACUUUGC GGAGGAAACUCC CU UCAAGGACAUCGUCCGGG UCCUUCUG | 8544 |
| 2807 | AGAAGGAG G CAAAGUGC | 2477 | GCACUUUG GGAGGAAACUCC CU UCAAGGACAUCGUCCGGG CUCCUUCU | 8545 |
| 2812 | GAGGCAAA G UGCCUAUC | 2478 | GAUAGGCA GGAGGAAACUCC CU UCAAGGACAUCGUCCGGG UUUGCCUC | 8546 |
| 2814 | GGCAAAGU G CCUAUCAA | 2479 | UUGAUAGG GGAGGAAACUCC CU UCAAGGACAUCGUCCGGG ACUUUGCC | 8547 |
| 2823 | CCUAUCAA G UGGAUGGC | 2480 | GCCAUCCA GGAGGAAACUCC CU UCAAGGACAUCGUCCGGG UUGAUAGG | 8548 |
| 2825 | UAUCAAGU G GAUGGCAU | 3486 | AUGCCAUC GGAGGAAACUCC CU UCAAGGACAUCGUCCGGG ACUUGAUA | 8549 |
| 2826 | AUCAAGUG G AUGGCAUU | 3487 | AAUGCCAU GGAGGAAACUCC CU UCAAGGACAUCGUCCGGG CACUUGAU | 8550 |
| 2829 | AAGUGGAU G GCAUUGGA | 3488 | UCCAAUGC GGAGGAAACUCC CU UCAAGGACAUCGUCCGGG AUCCACUU | 8551 |
| 2830 | AGUGGAUG G CAUUGGAA | 2481 | UUCCAAUG GGAGGAAACUCC CU UCAAGGACAUCGUCCGGG CAUCCACU | 8552 |
| 2835 | AUGGCAUU G GAAUCAAU | 3489 | AUUGAUUC GGAGGAAACUCC CU UCAAGGACAUCGUCCGGG AAUGCCAU | 8553 |
| 2836 | UGGCAUUG G AAUCAAUU | 3490 | AAUUGAUU GGAGGAAACUCC CU UCAAGGACAUCGUCCGGG CAAUGCCA | 8554 |
| 2852 | UUUACACA G AAUCUAUA | 3491 | UAUAGAUU GGAGGAAACUCC CU UCAAGGACAUCGUCCGGG UGUGUAAA | 8555 |
| 2868 | ACCCACCA G AGUGAUGU | 3492 | ACAUCACU GGAGGAAACUCC CU UCAAGGACAUCGUCCGGG UGGUGGGU | 8556 |
| 2870 | CCACCAGA G UGAUGUCU | 2482 | AGACAUCA GGAGGAAACUCC CU UCAAGGACAUCGUCCGGG UCUGGUGG | 8557 |
| 2872 | ACCAGAGU G AUGUCUGG | 3493 | CCAGACAU GGAGGAAACUCC CU UCAAGGACAUCGUCCGGG ACUCUGGU | 8558 |
| 2875 | AGAGUGAU G UCUGGAGC | 2483 | GCUCCAGA GGAGGAAACUCC CU UCAAGGACAUCGUCCGGG AUCACUCU | 8559 |
| 2879 | UGAUGUCU G GAGCUACG | 3494 | CGUAGCUC GGAGGAAACUCC CU UCAAGGACAUCGUCCGGG AGACAUCA | 8560 |
| 2880 | GAUGUCUG G AGCUACGG | 3495 | CCGUAGCU GGAGGAAACUCC CU UCAAGGACAUCGUCCGGG CAGACAUC | 8561 |
| 2882 | UGUCUGGA G CUACGGGG | 2484 | CCCCGUAG GGAGGAAACUCC CU UCAAGGACAUCGUCCGGG UCCAGACA | 8562 |
| 2887 | GGAGCUAC G GGGUGACC | 3496 | GGUCACCC GGAGGAAACUCC CU UCAAGGACAUCGUCCGGG GUAGCUCC | 8563 |
| 2888 | GAGCUACG G GGUGACCG | 3497 | CGGUCACC GGAGGAAACUCC CU UCAAGGACAUCGUCCGGG CGUAGCUC | 8564 |
| 2889 | AGCUACGG G GUGACCGU | 3498 | ACGGUCAC GGAGGAAACUCC CU UCAAGGACAUCGUCCGGG CCGUAGCU | 8565 |
| 2890 | GCUACGGG G UGACCGUU | 2485 | AACGGUCA GGAGGAAACUCC CU UCAAGGACAUCGUCCGGG CCCGUAGC | 8566 |
| 2892 | UACGGGGU G ACCGUUUG | 3499 | CAAACGGU GGAGGAAACUCC CU UCAAGGACAUCGUCCGGG ACCCCGUA | 8567 |
| 2896 | GGGUGACC G UUUGGGAG | 2486 | CUCCCAAA GGAGGAAACUCC CU UCAAGGACAUCGUCCGGG GGUCACCC | 8568 |
| 2900 | GACCGUUU G GGAGUUGA | 3500 | UCAACUCC GGAGGAAACUCC CU UCAAGGACAUCGUCCGGG AAACGGUC | 8569 |
| 2901 | ACCGUUUG G GAGUUGAU | 3501 | AUCAACUC GGAGGAAACUCC CU UCAAGGACAUCGUCCGGG CAAACGGU | 8570 |
| 2902 | CCGUUUGG G AGUUGAUG | 3502 | CAUCAACU GGAGGAAACUCC CU UCAAGGACAUCGUCCGGG CCAAACGG | 8571 |
| 2904 | GUUUGGGA G UUGAUGAC | 2487 | GUCAUCAA GGAGGAAACUCC CU UCAAGGACAUCGUCCGGG UCCCAAAC | 8572 |
| 2907 | UGGGAGUU G AUGACCUU | 3503 | AAGGUCAU GGAGGAAACUCC CU UCAAGGACAUCGUCCGGG AACUCCCA | 8573 |
| 2910 | GAGUUGAU G ACCUUUGG | 3504 | CCAAAGGU GGAGGAAACUCC CU UCAAGGACAUCGUCCGGG AUCAACUC | 8574 |
| 2917 | UGACCUUU G GAUCCAAG | 3505 | CUUGGAUC GGAGGAAACUCC CU UCAAGGACAUCGUCCGGG AAAGGUCA | 8575 |
| 2918 | GACCUUUG G AUCCAAGC | 3506 | GCUUGGAU GGAGGAAACUCC CU UCAAGGACAUCGUCCGGG CAAAGGUC | 8576 |
| 2925 | GGAUCCAA G CCAUAUGA | 2488 | UCAUAUGG GGAGGAAACUCC CU UCAAGGACAUCGUCCGGG UUGGAUCC | 8577 |
| 2932 | AGCCAUAU G ACGGAAUC | 3507 | GAUUCCGU GGAGGAAACUCC CU UCAAGGACAUCGUCCGGG AUAUGGCU | 8578 |
| 2935 | CAUAUGAC G GAAUCCCU | 3508 | AGGGAUUC GGAGGAAACUCC CU UCAAGGACAUCGUCCGGG GUCAUAUG | 8579 |
| 2936 | AUAUGACG G AAUCCCUG | 3509 | CAGGGAUU GGAGGAAACUCC CU UCAAGGACAUCGUCCGGG CGUCAUAU | 8580 |
| 2944 | GAAUCCCU G CCAGCGAG | 2489 | CUCGCUGG GGAGGAAACUCC CU UCAAGGACAUCGUCCGGG AGGGAUUC | 8581 |
| 2498 | CCCUGCCA G CGAGAUCU | 2490 | AGAUCUCG GGAGGAAACUCC CU UCAAGGACAUCGUCCGGG UGGCAGGG | 8582 |
| 2950 | CUGCCAGC G AGAUCUCC | 3510 | GGAGAUCU GGAGGAAACUCC CU UCAAGGACAUCGUCCGGG GCUGGCAG | 8583 |
| 2952 | GCCAGCGA G AUCUCCUC | 3511 | GAGGAGAU GGAGGAAACUCC CU UCAAGGACAUCGUCCGGG UCGCUGGC | 8584 |
| 2967 | UCCAUCCU G GAGAAAGG | 3512 | CCUUUCUC GGAGGAAACUCC CU UCAAGGACAUCGUCCGGG AGGAUGGA | 8585 |
| 2968 | CCAUCCUG G AGAAAGGA | 3513 | UCCUUUCU GGAGGAAACUCC CU UCAAGGACAUCGUCCGGG CAGGAUGG | 8586 |
| 2970 | AUCCUGGA G AAAGGAGA | 3514 | UCUCCUUU GGAGGAAACUCC CU UCAAGGACAUCGUCCGGG UCCAGGAU | 8587 |
| 2974 | UGGAGAAA G GAGAACGC | 3515 | GCGUUCUC GGAGGAAACUCC CU UCAAGGACAUCGUCCGGG UUUCUCCA | 8588 |
| 2975 | GGAGAAAG G AGAACGCC | 3516 | GGCGUUCU GGAGGAAACUCC CU UCAAGGACAUCGUCCGGG CUUUCUCC | 8589 |
| 2977 | AGAAAGGA G AACGCCUC | 3517 | GAGGCGUU GGAGGAAACUCC CU UCAAGGACAUCGUCCGGG UCCUUUCU | 8590 |
| 2981 | AGGAGAAC G CCUCCCUC | 2491 | GAGGGAGG GGAGGAAACUCC CU UCAAGGACAUCGUCCGGG GUUCUCCU | 8591 |
| 2991 | CUCCCUCA G CCACCCAU | 2492 | AUGGGUGG GGAGGAAACUCC CU UCAAGGACAUCGUCCGGG UGAGGGAG | 8592 |
| 3002 | ACCCAUAU G UACCAUCG | 2493 | CGAUGGUA GGAGGAAACUCC CU UCAAGGACAUCGUCCGGG AUAUGGGU | 8593 |
| 3010 | GUACCAUC G AUGUCUAC | 3518 | GUAGACAU GGAGGAAACUCC CU UCAAGGACAUCGUCCGGG GAUGGUAC | 8594 |
| 3013 | CCAUCGAU G UCUACAUG | 2494 | CAUGUAGA GGAGGAAACUCC CU UCAAGGACAUCGUCCGGG AUCGAUGG | 8595 |
| 3021 | GUCUACAU G AUCAUGGU | 3519 | ACCAUGAU GGAGGAAACUCC CU UCAAGGACAUCGUCCGGG AUGUAGAC | 8596 |
| 3027 | AUGAUCAU G GUCAAGUG | 3520 | CACUUGAC GGAGGAAACUCC CU UCAAGGACAUCGUCCGGG AUGAUCAU | 8597 |
| 3028 | UGAUCAUG G UCAAGUGC | 2495 | GCACUUGA GGAGGAAACUCC CU UCAAGGACAUCGUCCGGG CAUGAUCA | 8598 |
| 3033 | AUGGUCAA G UGCUGGAU | 2496 | AUCCAGCA GGAGGAAACUCC CU UCAAGGACAUCGUCCGGG UUGACCAU | 8599 |
| 3035 | GGUCAAGU G CUGGAUGA | 2497 | UCAUCCAG GGAGGAAACUCC CU UCAAGGACAUCGUCCGGG ACUUGACC | 8600 |
| 3038 | CAAGUGCU G GAUGAUAG | 3521 | CUAUCAUC GGAGGAAACUCC CU UCAAGGACAUCGUCCGGG AGCACUUG | 8601 |
| 3039 | AAGUGCUG G AUGAUAGA | 3522 | UCUAUCAU GGAGGAAACUCC CU UCAAGGACAUCGUCCGGG CAGCACUU | 8602 |
| 3042 | UGCUGGAU G AUAGACGC | 3523 | GCGUCUAU GGAGGAAACUCC CU UCAAGGACAUCGUCCGGG AUCCAGCA | 8603 |
| 3046 | GGAUGAUA G ACGCAGAU | 3524 | AUCUGCGU GGAGGAAACUCC CU UCAAGGACAUCGUCCGGG UAUCAUCC | 8604 |
| 3049 | UGAUAGAC G CAGAUAGU | 2498 | ACUAUCUG GGAGGAAACUCC CU UCAAGGACAUCGUCCGGG GUCUAUCA | 8605 |
| 3052 | UAGACGCA G AUAGUCGC | 3525 | GCGACUAU GGAGGAAACUCC CU UCAAGGACAUCGUCCGGG UGCGUCUA | 8606 |
| 3056 | CGCAGAUA G UCGCCCAA | 2499 | UUGGGCGA GGAGGAAACUCC CU UCAAGGACAUCGUCCGGG UAUCUGCG | 8607 |
| 3059 | AGAUAGUC G CCCAAAGU | 2500 | ACUUUGGG GGAGGAAACUCC CU UCAAGGACAUCGUCCGGG GACUAUCU | 8608 |
| 3066 | CGCCCAAA G UUCCGUGA | 2501 | UCACGGAA GGAGGAAACUCC CU UCAAGGACAUCGUCCGGG UUUGGGCG | 8609 |
| 3071 | AAAGUUCC G UGAGUUGA | 2502 | UCAACUCA GGAGGAAACUCC CU UCAAGGACAUCGUCCGGG GGAACUUU | 8610 |
| 3073 | AGUUCCGU G AGUUGAUC | 3526 | GAUCAACU GGAGGAAACUCC CU UCAAGGACAUCGUCCGGG ACGGAACU | 8511 |
| 3075 | UUCCGUGA G UUGAUCAU | 2503 | AUGAUCAA GGAGGAAACUCC CU UCAAGGACAUCGUCCGGG UCACGGAA | 8612 |
| 3078 | CGUGAGUU G AUCAUCGA | 3527 | UCGAUGAU GGAGGAAACUCC CU UCAAGGACAUCGUCCGGG AACUCACG | 8613 |
| 3085 | UGAUCAUC G AAUUCUCC | 3528 | GGAGAAUU GGAGGAAACUCC CU UCAAGGACAUCGUCCGGG GAUGAUCA | 8614 |
| 3099 | UCCAAAAU G GCCCGAGA | 3529 | UCUCGGGC GGAGGAAACUCC CU UCAAGGACAUCGUCCGGG AUUUUGGA | 8615 |
| 3100 | CCAAAAUG G CCCGAGAC | 2504 | GUGUCGGG GGAGAAACUCCC CU UCAAGGACAUCGUCCGGG CAUUUUGG | 8616 |
| 3104 | AAUGGCCC G AGACCCCC | 3530 | GGGGGUCU GGAGGAAACUCC CU UCAAGGACAUCGUCCGGG GGGCCAUU | 8617 |
| 3106 | UGGCCCGA G ACCCCCAG | 3531 | CUGGGGGU GGAGAAACUCCC CU UCAAGGACAUCGUCCGGG UCGGGCCA | 8618 |
| 3114 | CACCCCCA G CGCUACCU | 2505 | AGGUAGCG GGAGGAAACUCC CU UCAAGGACAUCGUCCGGG UGGGGGUC | 8619 |
| 3116 | CCCCCAGC G CUACCUUG | 2504 | CAAGGUAG GGAGAAACUCCC CU UCAAGGACAUCGUCCGGG GCUGGGGG | 8620 |
| 3124 | GCUACCUC G UCAUUCAG | 2507 | CUGAAUGA GGAGGAAACUCC CU UCAAGGACAUCGUCCGGG AAGGUAGC | 8621 |
| 3132 | GUCAUUCA G GGGGAUGA | 3532 | UCAUCCCC GGAGAAACUCCC CU UCAAGGACAUCGUCCGGG UGAAUGAC | 8622 |
| 3133 | UCAUUCAG G GGGAUGAA | 3533 | UUCAUCCC GGAGGAAACUCC CU UCAAGGACAUCGUCCGGG CUGAAUGA | 8623 |
| 3134 | CAUUCAGG G GGAUGAAA | 3534 | UUUCAUCC GGAGGAAACUCC CU UCAAGGACAUCGUCCGGG CCUGAAUG | 8624 |
| 3135 | AUUCAGGG G GAUGAAAG | 3535 | CUUUCAUC GGAGGAAACUCC CU UCAAGGACAUCGUCCGGG CCCUGAAU | 8625 |
| 3136 | UUCAGGGG G AUGAAAGA | 3536 | UCUUUCAU GGAGGAAACUCC CU UCAAGGACAUCGUCCGGG CCCCUGAA | 8626 |
| 3139 | AGGGGGAU G AAAGAAUG | 3537 | CAUUCUUU GGAGGAAACUCC CU UCAAGGACAUCGUCCGGG AUCCCCCU | 8627 |
| 3143 | GGAUGAAA G AAUGCAUU | 3538 | AAUGCAUU GGAGGAAACUCC CU UCAAGGACAUCGUCCGGG UUUCAUCC | 8628 |
| 3147 | GAAAGAAU G CAUUUGCC | 2508 | GGCAAAUG GGAGGAAACUCC CU UCAAGGACAUCGUCCGGG AUUCUUUC | 8629 |
| 3153 | AUGCAUUU G CCAAGUCC | 2509 | GGACUUGG GGAGGAAACUCC CU UCAAGGACAUCGUCCGGG AAAUGCAU | 8630 |
| 3158 | UUUGCCAA G UCCUACAG | 2510 | CUGUAGGA GGAGGAAACUCC CU UCAAGGACAUCGUCCGGG UUGGCAAA | 8631 |
| 3166 | GUCCUACA G ACUCCAAC | 3539 | GUUGGAGU GGAGGAAACUCC CU UCAAGGACAUCGUCCGGG UGUAGGAC | 8632 |
| 3182 | CUUCUACC G UGCCCUGA | 2511 | UCAGGGCA GGAGGAAACUCC CU UCAAGGACAUCGUCCGGG GGUAGAAG | 8633 |
| 3184 | UCUACCGU G CCCUGAUG | 2512 | CAUCAGGG GGAGGAAACUCC CU UCAAGGACAUCGUCCGGG ACGGUAGA | 8634 |
| 3189 | CGUGCCCU G AUGGAUGA | 3540 | UCAUCCAU GGAGGAAACUCC CU UCAAGGACAUCGUCCGGG AGGGCACG | 8635 |
| 3192 | GCCCUGAU G GAUGAAGA | 3541 | UCUUCAUC GGAGGAAACUCC CU UCAAGGACAUCGUCCGGG AUCAGGGC | 8636 |
| 3193 | CCCUGAUG G AUGAAGAA | 3542 | UUCUUCAU GGAGGAAACUCC CU UCAAGGACAUCGUCCGGG CAUCAGGG | 8637 |
| 3196 | UGAUGGAU G AAGAAGAC | 3543 | GUCUUCUU GGAGGAAACUCC CU UCAAGGACAUCGUCCGGG AUCCAUCA | 8638 |
| 3199 | UGGAUGAA G AAGACAUG | 3544 | CAUGUCUU GGAGGAAACUCC CU UCAAGGACAUCGUCCGGG UUCAUCCA | 8639 |
| 3202 | AUGAAGAA G ACAUGGAC | 3545 | GUCCAUGU GGAGGAAACUCC CU UCAAGGACAUCGUCCGGG UUCUUCAU | 8640 |
| 3207 | GAAGACAU G GACGACGU | 3546 | ACGUCGUC GGAGGAAACUCC CU UCAAGGACAUCGUCCGGG AUGUCUUC | 8641 |
| 3208 | AAGACAUG G ACGACGUG | 3547 | CACGUCGU GGAGGAAACUCC CU UCAAGGACAUCGUCCGGG CAUGUCUU | 8642 |
| 3211 | ACAUGGAC G ACGUGGUG | 3548 | CACCACGU GGAGGAAACUCC CU UCAAGGACAUCGUCCGGG GUCCAUGU | 8643 |
| 3214 | UGGACGAC G UGGUGGAU | 2513 | AUCCACCA GGAGGAAACUCC CU UCAAGGACAUCGUCCGGG GUCGUCCA | 8644 |
| 3216 | GACGACGU G GUGGAUGC | 3549 | GCAUCCAC GGAGGAAACUCC CU UCAAGGACAUCGUCCGGG ACGUCGUC | 8645 |
| 3217 | ACGACGUG G UGGAUGCC | 2514 | GGCAUCCA GGAGGAAACUCC CU UCAAGGACAUCGUCCGGG CACGUCGU | 8646 |
| 3219 | GACGUGGU G GAUGCCGA | 3550 | UCGGCAUC GGAGGAAACUCC CU UCAAGGACAUCGUCCGGG ACCACGUC | 8647 |
| 3220 | ACGUGGUG G AUGCCGAC | 3551 | GUCGGCAU GGAGGAAACUCC CU UCAAGGACAUCGUCCGGG CACCACGU | 8648 |
| 3223 | UGGUGGAU G CCGACGAG | 2515 | CUCGUCGG GGAGGAAACUCC CU UCAAGGACAUCGUCCGGG AUCCACCA | 8649 |
| 3226 | UGGAUGCC G ACGAGUAC | 3552 | GUACUCGU GGAGGAAACUCC CU UCAAGGACAUCGUCCGGG GGCAUCCA | 8650 |
| 3229 | AUGCCGAC G AGUACCUC | 3553 | GAGGUACU GGAGGAAACUCC CU UCAAGGACAUCGUCCGGG GUCGGCAU | 8651 |
| 3231 | GCCGACGA G UACCUCAU | 3548 | AUGAGGUA GGAGGAAACUCC CU UCAAGGACAUCGUCCGGG UCGUCGGC | 8652 |
| 3246 | AUCCCACA G CAGGGCUU | 2517 | AAGCCCUG GGAGGAAACUCC CU UCAAGGACAUCGUCCGGG UGUGGGAU | 8653 |
| 3249 | CCACAGCA G GGCUUCUU | 3554 | AAGAAGCC GGAGGAAACUCC CU UCAAGGACAUCGUCCGGG UGCUGUGG | 8654 |
| 3250 | CACAGCAG G GCUUCUUC | 3555 | GAAGAAGC GGAGGAAACUCC CU UCAAGGACAUCGUCCGGG CUGCUGUG | 8655 |
| 3251 | ACAGCAGG G CUUCUUCA | 2518 | UGAAGAAG GGAGGAAACUCC CU UCAAGGACAUCGUCCGGG CCUGCUGU | 8656 |
| 3260 | CUUCUUCA G CAGCCCCU | 2519 | AGGGGCUG GGAGGAAACUCC CU UCAAGGACAUCGUCCGGG UGAAGAAG | 8657 |
| 3263 | CUUCAGCA G CCCCUCCA | 2520 | UGGAGGGG GGAGGAAACUCC CU UCAAGGACAUCGUCCGGG UGCUGAAG | 8658 |
| 3273 | CCCUCCAC G UCACGGAC | 2521 | GUCCGUGA GGAGGAAACUCC CU UCAAGGACAUCGUCCGGG GUGGAGGG | 8659 |
| 3278 | CACGUCAC G GACUCCCC | 3553 | GGGGAGUC GGAGGAAACUCC CU UCAAGGACAUCGUCCGGG GUGACGUG | 8660 |
| 3279 | ACGUCACG G ACUCCCCU | 3557 | AGGGGAGU GGAGGAAACUCC CU UCAAGGACAUCGUCCGGG CGUGACGU | 8661 |
| 3291 | CCCCUCCU G AGCUCUCU | 3558 | AGAGAGCU GGAGGAAACUCC CU UCAAGGACAUCGUCCGGG AGGAGGGG | 8662 |
| 3293 | CCUCCUGA G CUCUCUGA | 2522 | UCAGAGAG GGAGGAAACUCC CU UCAAGGACAUCGUCCGGG UCAGGAGG | 8663 |
| 3300 | AGCUCUCU G AGUGCAAC | 3559 | GUUGCACU GGAGGAAACUCC CU UCAAGGACAUCGUCCGGG AGAGAGCU | 8664 |
| 3302 | CUCUCUGA G UGCAACCA | 2523 | UGGUUGCA GGAGGAAACUCC CU UCAAGGACAUCGUCCGGG UCAGAGAG | 8665 |
| 3304 | CUCUGAGU G CAACCAGC | 2524 | GCUGGUUG GGAGGAAACUCC CU UCAAGGACAUCGUCCGGG ACUCAGAG | 8666 |
| 3311 | UGCAACCA G CAACAAUU | 2525 | AAUUGUUG GGAGGAAACUCC CU UCAAGGACAUCGUCCGGG UGGUUGCA | 8667 |
| 3325 | AUUCCACC G UGGCUUGC | 2526 | GCAAGCCA GGAGGAAACUCC CU UCAAGGACAUCGUCCGGG GGUGGAAU | 8668 |
| 3327 | UCCACCGU G GCUUGCAU | 3560 | AUGCAAGC GGAGGAAACUCC CU UCAAGGACAUCGUCCGGG ACGGUGGA | 8669 |
| 3328 | CCACCGUG G CUUGCAUU | 2527 | AAUGCAAG GGAGGAAACUCC CU UCAAGGACAUCGUCCGGG CACGGUGG | 8670 |
| 3332 | CGUGGCUU G CAUUGAUA | 2528 | UAUCAAUG GGAGGAAACUCC CU UCAAGGACAUCGUCCGGG AAGCCACG | 8671 |
| 3337 | CUUGCAUU G AUAGAAAU | 3561 | AUUUCUAU GGAGGAAACUCC CU UCAAGGACAUCGUCCGGG AAUGCAAG | 8672 |
| 3341 | CAUUGAUA G AAAUGGGC | 3562 | GCCCAUUU GGAGGAAACUCC CU UCAAGGACAUCGUCCGGG UAUCAAUG | 8673 |
| 3346 | AUAGAAAU G GGCUGCAA | 3563 | UUGCAGCC GGAGGAAACUCC CU UCAAGGACAUCGUCCGGG AUUUCCAU | 8674 |
| 3347 | UAGAAAUG G GCUGCAAA | 3564 | UUUGCAGC GGAGGAAACUCC CU UCAAGGACAUCGUCCGGG CAUUUCUA | 8675 |
| 3348 | AGAAAUGG G CUGCAAAG | 2529 | CUUUGCAG GGAGGAAACUCC CU UCAAGGACAUCGUCCGGG CCAUUUCU | 8676 |
| 3351 | AAUGGGCU G CAAAGCUG | 2530 | CAGCUUUG GGAGGAAACUCC CU UCAAGGACAUCGUCCGGG AGCCCAUU | 8677 |
| 3356 | GCUGCAAA G CUGUCCCA | 2531 | UGGGACAG GGAGGAAACUCC CU UCAAGGACAUCGUCCGGG UUUGCAGC | 8678 |
| 3359 | GCAAAGCU G UCCCAUCA | 2532 | UGAUGGGA GGAGGAAACUCC CU UCAAGGACAUCGUCCGGG AGCUUUGC | 8679 |
| 3369 | CCCAUCAA G GAAGACAG | 3565 | CUGUCUUC GGAGGAAACUCC CU UCAAGGACAUCGUCCGGG UUGAUGGG | 8680 |
| 3370 | CCAUCAAG G AAGACAGC | 3566 | GCUGUCUU GGAGGAAACUCC CU UCAAGGACAUCGUCCGGG CUUGAUGG | 8681 |
| 3373 | UCAAGGAA G ACAGCUUC | 3567 | GAAGCUGU GGAGGAAACUCC CU UCAAGGACAUCGUCCGGG UUCCUUGA | 8682 |
| 3377 | GGAAGACA G CUUCUUGC | 2533 | GCAAGAAG GGAGGAAACUCC CU UCAAGGACAUCGUCCGGG UGUCUUCC | 8683 |
| 3384 | AGCUUCUU G CAGCGAUA | 2534 | UAUCGCUG GGAGGAAACUCC CU UCAAGGACAUCGUCCGGG AAGAAGCU | 8684 |
| 3387 | UUCUUGCA G CGAUACAG | 2535 | CUGUAUCG GGAGGAAACUCC CU UCAAGGACAUCGUCCGGG UGCAAGAA | 8686 |
| 3389 | CUUGCAGC G AUACAGCU | 3568 | AGCUGUAU GGAGGAAACUCC CU UCAAGGACAUCGUCCGGG GCUGCAAG | 8686 |
| 3395 | GCGAUACA G CUCAGACC | 2536 | GGUCUGAG GGAGGAAACUCC CU UCAAGGACAUCGUCCGGG UGUAUCGC | 8687 |
| 3400 | ACAGCUCA G ACCCCACA | 3569 | UGUGGGGU GGAGGAAACUCC CU UCAAGGACAUCGUCCGGG UGAGCUGU | 8688 |
| 3409 | ACCCCACA G GCGCCUUG | 3570 | CAAGGCGC GGAGGAAACUCC CU UCAAGGACAUCGUCCGGG UGUGGGGU | 8689 |
| 3410 | CCCCACAG G CGCCUUGA | 2537 | UCAAGGCG GGAGGAAACUCC CU UCAAGGACAUCGUCCGGG CUGUGGGG | 8690 |
| 3412 | CCACAGGC G CCUUGACU | 2538 | AGUCAAGG GGAGGAAACUCC CU UCAAGGACAUCGUCCGGG GCCUGUGG | 8691 |
| 3417 | GGCGCCUU G ACUGAGGA | 3571 | UCCUCAGU GGAGGAAACUCC CU UCAAGGACAUCGUCCGGG AAGGCGCC | 8692 |
| 3421 | CCUUGACU G AGGACAGC | 3572 | GCUGUCCU GGAGGAAACUCC CU UCAAGGACAUCGUCCGGG AGUCAAGG | 8693 |
| 3423 | UUGACUGA G GACAGCAU | 3573 | AUGCUGUC GGAGGAAACUCC CU UCAAGGACAUCGUCCGGG UCAGUCAA | 8694 |
| 3424 | UGACUGAG G ACAGCAUA | 3574 | UAUGCUGU GGAGGAAACUCC CU UCAAGGACAUCGUCCGGG CUCAGUCA | 8695 |
| 3428 | UGAGGACA G CAUAGACG | 2539 | CGUCUAUG GGAGGAAACUCC CU UCAAGGACAUCGUCCGGG UGUCCUCA | 8696 |
| 3433 | ACAGCAUA G ACGACACC | 3575 | GGUGUCGU GGAGGAAACUCC CU UCAAGGACAUCGUCCGGG UAUGCUGU | 8697 |
| 3436 | GCAUAGAC G ACACCUUC | 3576 | GAAGGUGU GGAGGAAACUCC CU UCAAGGACAUCGUCCGGG GUCUAUGC | 8698 |
| 3451 | UCCUCCCA G UGCCUGAA | 2540 | UUCAGGCA GGAGGAAACUCC CU UCAAGGACAUCGUCCGGG UGGGAGGA | 8699 |
| 3453 | CUCCCAGU G CCUGAAUA | 2541 | UAUUCAGG GGAGGAAACUCC CU UCAAGGACAUCGUCCGGG ACUGGGAG | 8700 |
| 3457 | CAGUGCCU G AAUACAUA | 3577 | UAUGUAUU GGAGGAAACUCC CU UCAAGGACAUCGUCCGGG AGGCACUG | 8701 |
| 3471 | AUAAACCA G UCCGUUCC | 2542 | GGAACGGA GGAGGAAACUCC CU UCAAGGACAUCGUCCGGG UGGUUUAU | 8702 |
| 3475 | ACCAGUCC G UUCCCAAA | 2543 | UUUGGGAA GGAGGAAACUCC CU UCAAGGACAUCGUCCGGG GGACUGGU | 8703 |
| 3485 | UCCCAAAA G GCCCGCUG | 3578 | CAGCGGGC GGAGGAAACUCC CU UCAAGGACAUCGUCCGGG UUUUGGGA | 8704 |
| 3486 | CCCAAAAG G CCCGCUGG | 2544 | CCAGCGGG GGAGGAAACUCC CU UCAAGGACAUCGUCCGGG CUUUUGGG | 8705 |
| 3490 | AAAGGCCC G CUGGCUCU | 2545 | AGAGCCAG GGAGGAAACUCC CU UCAAGGACAUCGUCCGGG GGGCCUUU | 8706 |
| 3493 | GGCCCGCU G GCUCUGUG | 3579 | CACAGAGC GGAGGAAACUCC CU UCAAGGACAUCGUCCGGG AGCGGGCC | 8707 |
| 3494 | GCCCGCUG G CUCUGUGC | 2546 | GCACAGAG GGAGGAAACUCC CU UCAAGGACAUCGUCCGGG CAGCGGGC | 8708 |
| 3499 | CUGGCUCU G UGCAGAAU | 2547 | ACUCUCCA GGAGGAAACUCC CU UCAAGGACAUCGUCCGGG AGAGCCAG | 8709 |
| 3501 | GGCUCUGU G CAGAAUCC | 2548 | GGAUUCUG GGAGGAAACUCC CU UCAAGGACAUCGUCCGGG ACAGAGCC | 8710 |
| 3504 | UCUGUGCA G AAUCCUGU | 3580 | ACAGGAUU GGAGGAAACUCC CU UCAAGGACAUCGUCCGGG UGCACAGA | 8711 |
| 3511 | AGAAUCCU G UCUAUCAC | 2549 | GUGAUAGA GGAGGAAACUCC CU UCAAGGACAUCGUCCGGG AGGAUUCU | 8712 |
| 3525 | CACAAUCA G CCUCUGAA | 2550 | UUCAGAGG GGAGGAAACUCC CU UCAAGGACAUCGUCCGGG UGAUUGUG | 8713 |
| 3531 | CAGCCUCU G AACCCCGC | 3581 | GCGGGGUU GGAGGAAACUCC CU UCAAGGACAUCGUCCGGG AGAGGCUG | 8714 |
| 3538 | UGAACCCC G CGCCCAGC | 2551 | GCUGGGCG GGAGGAAACUCC CU UCAAGGACAUCGUCCGGG GGGGUUCA | 8715 |
| 3540 | AACCCCGC G CCCAGCAG | 2552 | CUGCUCGG CGAGGAAACUCC CU UCAAGGACAUCGUCCCGG GCGGGGUU | 8716 |
| 3545 | CGCGCCCA G CAGAGACC | 2553 | GGUCUCUG GGAGGAAACUCC CU UCAAGGACAUCGUCCGGG UGGGCGCG | 8717 |
| 3548 | GCCCACCA G AGACCCAC | 3582 | GUGGGUCU GGAGGAAACUCC CU UCAAGGACAUCGUCCGGG UGCUGGGC | 8718 |
| 3550 | CCAGCACA G ACCCACAC | 3583 | GUGUGGGU GGACGAAACUCC CU UCAAGGACAUCGUCCGGG UCUGCUGG | 8719 |
| 3564 | CACUACCA G GACCCCCA | 3584 | UGGGGGUC GGAGGAAACUCC CU UCAAGGACAUCGUCCGGG UGGUAGUG | 8720 |
| 3565 | ACUACCAG G ACCCCCAC | 3585 | GUGGCCGU GGAGGAAACUCC CU UCAAGGACAUCGUCCCGG CUGGUAGU | 8721 |
| 3575 | CCCCCACA G CACUGCAG | 2554 | CUGCAGUG GGAGGAAACUCC CU UCAAGGACAUCGUCCGGG UGUGGGGG | 8722 |
| 3580 | ACAGCACU G CAGUGGGC | 2555 | GCCCACUG GGAGGAAACUCC CU UCAAGGACAUCGUCCGGG AGUGCUGU | 8723 |
| 3583 | GCACUGCA G UGGGCAAC | 2556 | GUUGCCCA CGAGGAAACUCC CU UCAAGGACAUCGUCCGGG UGCAGUGC | 8724 |
| 3585 | ACUGCAGU G GGCAACCC | 3586 | CGGUUCCC GGAGGAAACUCC CU UCAAGGACAUCGUCCGGG ACUGCAGU | 8725 |
| 3586 | CUGCAGUG G GCAACCCC | 3587 | GGGGUUGC CGAGGAAACUCC CU UCAAGGACAUCGUCCCGG CACUGCAG | 8726 |
| 3587 | UGCAGUGG G CAACCCCG | 2557 | CGGGGUUG GGAGGAAACUCC CU UCAAGGACAUCGUCCGGG CCACUGCA | 8727 |
| 3595 | GCAACCCC G AGUAUCUC | 3588 | GAGAUACU GGAGGAAACUCC CU UCAAGGACAUCGUCCGGG GGGGUUGC | 8728 |
| 3597 | AACCCCGA G UAUCUCAA | 2558 | UUGAGAUA GGACGAAACUCC CU UCAAGGACAUCGUCCGGG UCGGGGUU | 8729 |
| 3610 | UCAACACU G UCCAGCCC | 2559 | GGGCUGGA GGAGGAAACUCC CU UCAAGGACAUCGUCCGGG AGUGUUGA | 8730 |
| 3615 | ACUGUCCA G CCCACCUG | 2560 | CAGGUGGG GGAGGAAACUCC CU UCAAGGACAUCGUCCCGG UGGACAGU | 8731 |
| 3623 | GCCCACCU G UGUCAACA | 2561 | UGUUGACA GGAGGAAACUCC CU UCAAGGACAUCGUCCGGG AGGUGGGC | 8732 |
| 3625 | CCACCUGU G UCAACAGC | 2562 | GCUGUUGA GGAGGAAACUCC CU UCAAGGACAUCGUCCGGG ACAGGUGG | 8733 |
| 3632 | UGUCAACA G CACAUUCG | 2563 | CGAAUGUG GGAGGAAACUCC CU UCAAGGACAUCGUCCGGG UGUUGACA | 8734 |
| 3640 | GCACAUUC G ACAGCCCU | 3589 | AGGGCUGU GGAGGAAACUCC CU UCAAGGACAUCGUCCGGG GAAUGUGC | 8735 |
| 3644 | AUUCGACA G CCCUGCCC | 2564 | GGGCAGGG GGAGGAAACUCC CU UCAAGGACAUCGUCCGGG UGUCGAAU | 8736 |
| 3649 | ACAGCCCU G CCCACUGG | 2565 | CCAGUGGG GGAGGAAACUCC CU UCAAGGACAUCGUCCGGG AGGGCUGU | 8737 |
| 3656 | UGCCCACU G GGCCCAGA | 3590 | UCUGGGCC GGAGGAAACUCC CU UCAAGGACAUCGUCCGGG AGUGGGCA | 8738 |
| 3657 | GCCCACUG G GCCCAGAA | 3591 | UUCUGGGC GGAGGAAACUCC CU UCAAGGACAUCGUCCGGG CAGUGGGC | 8739 |
| 3658 | CCCACUGG G CCCAGAAA | 2566 | UUUCUGGG GGAGGAAACUCC CU UCAAGGACAUCGUCCGGG CCAGUGGG | 8740 |
| 3663 | UGGGCCCA G AAAGGCAG | 3592 | CUGCCUUU GGAGGAAACUCC CU UCAAGGACAUCGUCCGGG UGGGCCCA | 8741 |
| 3667 | CCCAGAAA G GCAGCCAC | 3593 | GUGGCUGC GGAGGAAACUCC CU UCAAGGACAUCGUCCGGG UUUCUGGG | 8742 |
| 3668 | CCAGAAAG G CAGCCACC | 2567 | GGUGGCUG GGAGGAAACUCC CU UCAAGGACAUCGUCCGGG CUUUCUGG | 8743 |
| 3671 | GAAAGGCA G CCACCAAA | 2568 | UUUGGUGG GGAGGAAACUCC CU UCAAGGACAUCGUCCGGG UGCCUUUC | 8744 |
| 3683 | CCAAAUUA G CCUGGACA | 2569 | UGUCCAGG GGAGGAAACUCC CU UCAAGGACAUCGUCCGGG UAAUUUGG | 8745 |
| 3687 | AUUAGCCU G GACAACCC | 3594 | GGGUUGUC GGAGGAAACUCC CU UCAAGGACAUCGUCCGGG AGGCUAAU | 8746 |
| 3688 | UUAGCCUG G ACAACCCU | 3595 | AGGGUUGU GGAGGAAACUCC CU UCAAGGACAUCGUCCGGG CAGGCUAA | 8747 |
| 3697 | ACAACCCU G ACUACCAG | 3596 | CUGGUAGU GGAGGAAACUCC CU UCAAGGACAUCGUCCGGG AGGGUUGU | 8748 |
| 3705 | GACUACCA G CAGGACUU | 2570 | AAGUCCUG GGAGGAAACUCC CU UCAAGGACAUCGUCCGGG UGGUAGUC | 8749 |
| 3708 | UACCAGCA G GACUUCUU | 3597 | AAGAAGUC GGAGGAAACUCC CU UCAAGGACAUCGUCCGGG UGCUGGUA | 8750 |
| 3709 | ACCAGCAG G ACUUCUUU | 3598 | AAAGAAGU GGAGGAAACUCC CU UCAAGGACAUCGUCCGGG CUGCUGGU | 8751 |
| 3723 | UUUCCCAA G GAAGCCAA | 3599 | UUGGCUUC GGAGGAAACUCC CU UCAAGGACAUCGUCCGGG UUGGGAAA | 8752 |
| 3724 | CCAAGGAA G AAGCCAAG | 3600 | CUUGGCUU GGAGGAAACUCC CU UCAAGGACAUCGUCCGGG CUUGGGAA | 8753 |
| 3727 | CCAAGGAA G CCAAGCCA | 2571 | UGGCUUGG GGAGGAAACUCC CU UCAAGGACAUCGUCCGGG UUCCUUGG | 8754 |
| 3732 | GAAGCCAA G CCAAAUGG | 2572 | CCAUUUGG GGAGGAAACUCC CU UCAAGGACAUCGUCCGGG UUGGCUUC | 8755 |
| 3739 | AGCCAAAU G GCAUCUUU | 3601 | AAAGAUGC GGAGGAAACUCC CU UCAAGGACAUCGUCCGGG AUUUGGCU | 8756 |
| 3740 | GCCAAAUG G CAUCUUUA | 2573 | UAAAGAUG GGAGGAAACUCC CU UCAAGGACAUCGUCCGGG CAUUUGGC | 8757 |
| 3750 | AUCUUUAA G GGCUCCAC | 3602 | GUGGAGCC GGAGGAAACUCC CU UCAAGGACAUCGUCCGGG UUAAAGAU | 8758 |
| 3751 | UCUUUAAG G GCUCCACA | 3603 | UGUGGAGC GGAGGAAACUCC CU UCAAGGACAUCGUCCGGG CUUAAAGA | 8759 |
| 3752 | CUUUAAGG G CUCCACAG | 2574 | CUGUGGAG GGAGGAAACUCC CU UCAAGGACAUCGUCCGGG CCUUAAAG | 8760 |
| 3760 | GCUCCACA G CUGAAAAU | 2575 | AUUUUCAG GGAGGAAACUCC CU UCAAGGACAUCGUCCGGG UGUGGAGC | 8761 |
| 3763 | CCACAGCU G AAAAUGCA | 3604 | UGCAUUUU GGAGGAAACUCC CU UCAAGGACAUCGUCCGGG AGCUGUGG | 8762 |
| 3769 | CUGAAAAU G CAGAAUAC | 2576 | GUAUUCUG GGAGGAAACUCC CU UCAAGGACAUCGUCCGGG AUUUUCAG | 8763 |
| 3772 | AAAAUGCA G AAUACCUA | 3605 | UAGGUAUU GGAGGAAACUCC CU UCAAGGACAUCGUCCGGG UGCAUUUU | 8764 |
| 3782 | AUACCUAA G GGUCGCGC | 3606 | GCGCGACC GGAGGAAACUCC CU UCAAGGACAUCGUCCGGG UUAGGUAU | 8765 |
| 3783 | UACCUAAG G GUCGCGCC | 3607 | GGCGCGAC GGAGGAAACUCC CU UCAAGGACAUCGUCCGGG CUUAGGUA | 8766 |
| 3784 | ACCUAAGG G UCGCGCCA | 2577 | UGGCGCGA GGAGGAAACUCC CU UCAAGGACAUCGUCCGGG CCUUAGGU | 8767 |
| 3787 | UAAGGGUC G CGCCACAA | 2578 | UUGUGGCG GGAGGAAACUCC CU UCAAGGACAUCGUCCGGG GACCCUUA | 8768 |
| 3789 | AGGGUCGC G CCACAAAG | 2579 | CUUUGUGG GGAGGAAACUCC CU UCAAGGACAUCGUCCGGG GCGACCCU | 8769 |
| 3797 | GCCACAAA G CAGUGAAU | 2580 | AUUCACUG GGAGGAAACUCC CU UCAAGGACAUCGUCCGGG UUUGUGGC | 8770 |
| 3800 | ACAAAGCA G UGAAUUUA | 2581 | UAAAUUCA GGAGGAAACUCC CU UCAAGGACAUCGUCCGGG UGCUUUGU | 8771 |
| 3802 | AAAGCAGU G AAUUUAUU | 3608 | AAUAAAUU GGAGGAAACUCC CU UCAAGGACAUCGUCCGGG ACUGCUUU | 8772 |
| 3811 | AAUUUAUU G GAGCAUGA | 3609 | UCAUGCUC GGAGGAAACUCC CU UCAAGGACAUCGUCCGGG AAUAAAUU | 8773 |
| 3812 | AUUUAUUG G AGCAUGAC | 3610 | GUCAUGCU GGAGGAAACUCC CU UCAAGGACAUCGUCCGGG CAAUAAAU | 8774 |
| 3814 | UUAUUGGA G CAUGACCA | 2582 | UGGUCAUG GGAGGAAACUCC CU UCAAGGACAUCGUCCGGG UCCAAUAA | 8775 |
| 3818 | UGGAGCAU G ACCACGGA | 3611 | UCCGUGGU GGAGGAAACUCC CU UCAAGGACAUCGUCCGGG AUGCUCCA | 8776 |
| 3824 | AUGACCAC G GAGGAUAG | 3612 | CUAUCCUC GGAGGAAACUCC CU UCAAGGACAUCGUCCGGG GUGGUCAU | 8777 |
| 3825 | UGACCACG G AGGAUAGU | 3613 | ACUAUCCU GGAGGAAACUCC CU UCAAGGACAUCGUCCGGG CGUGGUCA | 8778 |
| 3827 | ACCACGGA G GAUAGUAU | 3614 | AUACUAUC GGAGGAAACUCC CU UCAAGGACAUCGUCCGGG UCCGUGGU | 8779 |
| 3828 | CCACGGAG G AUAGUAUG | 3615 | CAUACUAU GGAGGAAACUCC CU UCAAGGACAUCGUCCGGG CUCCGUGG | 8780 |
| 3832 | GGAGGAUA G UAUGAGCC | 2583 | GGCUCAUA GGAGGAAACUCC CU UCAAGGACAUCGUCCGGG UAUCCUCC | 8781 |
| 3836 | GAUAGUAU G AGCCCUAA | 3616 | UUAGGGCU GGAGGAAACUCC CU UCAAGGACAUCGUCCGGG AUACUAUC | 8782 |
| 3838 | UAGUAUGA G CCCUAAAA | 2584 | UUUUAGGG GGAGGAAACUCC CU UCAAGGACAUCGUCCGGG UCAUACUA | 8783 |
| 3852 | AAAAUCCA G ACUCUUUC | 3617 | GAAAGAGU GGAGGAAACUCC CU UCAAGGACAUCGUCCGGG UGGAUUUU | 8784 |
| 3861 | ACUCUUUC G AUACCCAG | 3618 | CUGGGUAU GGAGGAAACUCC CU UCAAGGACAUCGUCCGGG GAAAGAGU | 8785 |
| 3869 | GAUACCCA G GACCAAGC | 3619 | GCUUGGUC GGAGGAAACUCC CU UCAAGGACAUCGUCCGGG UGGGUAUC | 8786 |
| 3870 | AUACCCAG G ACCAAGCC | 3620 | GGCUUGGU GGAGGAAACUCC CU UCAAGGACAUCGUCCGGG CUGGGUAU | 8787 |
| 3876 | AGGACCAA G CCACAGCA | 2585 | UGCUGUGG GGAGGAAACUCC CU UCAAGGACAUCGUCCGGG UUGGUCCU | 8788 |
| 3882 | AAGCCACA G CAGGUCCU | 2586 | AGGACCUG GGAGGAAACUCC CU UCAAGGACAUCGUCCGGG UGUGGCUU | 8789 |
| 3885 | CCACAGCA G GUCCUCCA | 3621 | UGGAGGAC GGAGGAAACUCC CU UCAAGGACAUCGUCCGGG UGCUGUGG | 8790 |
| 3886 | CACAGCAG G UCCUCCAU | 2587 | AUGGAGGA GGAGGAAACUCC CU UCAAGGACAUCGUCCGGG CUGCUGUG | 8791 |
| 3902 | UCCCAACA G CCAUGCCC | 2588 | GGGCAUGG GGAGGAAACUCC CU UCAAGGACAUCGUCCGGG UGUUGGGA | 8792 |
| 3907 | ACAGCCAU G CCCGCAUU | 2588 | AAUGCGGG GGAGGAAACUCC CU UCAAGGACAUCGUCCGGG AUGGCUGU | 8793 |
| 3911 | CCAUGCCC G CAUUAGCU | 2590 | AGCUAAUG GGAGGAAACUCC CU UCAAGGACAUCGUCCGGG GGGCAUGG | 8794 |
| 3917 | CCGCAUUA G CUCUUAGA | 2591 | UCUAAGAG GGAGGAAACUCC CU UCAAGGACAUCGUCCGGG UAAUGCGG | 8795 |
| 3924 | AGCUCUUA G ACCCACAG | 3622 | CUGUGGGU GGAGGAAACUCC CU UCAAGGACAUCGUCCGGG UAAGAGCU | 8796 |
| 3932 | GACCCACA G ACUGGUUU | 3623 | AAACCAGU GGAGGAAACUCC CU UCAAGGACAUCGUCCGGG UGUGGGUC | 8797 |
| 3936 | CACAGACU G GUUUUGCA | 3624 | UGCAAAAC GGAGGAAACUCC CU UCAAGGACAUCGUCCGGG AGUCUGUG | 8798 |
| 3937 | ACAGACUG G UUUUGCAA | 2592 | UUGCAAAA GGAGGAAACUCC CU UCAAGGACAUCGUCCGGG CAGUCUGU | 8799 |
| 3942 | CUGGUUUU G CAACGUUU | 2593 | AAACGUUG GGAGGAAACUCC CU UCAAGGACAUCGUCCGGG AAAACCAG | 8800 |
| 3947 | UUUGCAAC G UUUACACC | 2594 | GGUGUAAA GGAGGAAACUCC CU UCAAGGACAUCGUCCGGG GUUGCAAA | 8801 |
| 3956 | UUUACACC G ACUAGCCA | 3624 | UGGCUAGU GGAGGAAACUCC CU UCAAGGACAUCGUCCGGG GGUGUAAA | 8802 |
| 3961 | ACCGACUA G CCAGGAAG | 2595 | CUUCCUGG GGAGGAAACUCC CU UCAAGGACAUCGUCCGGG UAGUCGGU | 8803 |
| 3965 | ACUAGCCA G GAAGUACU | 3926 | AGUACUUC GGAGGAAACUCC CU UCAAGGACAUCGUCCGGG UGGCUAGU | 8804 |
| 3966 | CUAGCCAG G AAGUACUU | 3627 | AAGUACUU GGAGGAAACUCC CU UCAAGGACAUCGUCCGGG CUGGCUAG | 8805 |
| 3969 | GCCAGGAA G UACUUCCA | 2596 | UGGAAGUA GGAGGAAACUCC CU UCAAGGACAUCGUCCGGG UUCCUGGC | 8806 |
| 3982 | UCCACCUC G GGCACAUU | 3628 | AAUGUGCC GGAGGAAACUCC CU UCAAGGACAUCGUCCGGG GAGGUGGA | 8807 |
| 3983 | CCACCUCG G GCACAUUU | 3629 | AAAUGUGC GGAGGAAACUCC CU UCAAGGACAUCGUCCGGG CGAGGUGG | 8808 |
| 3984 | CACCUCGG G CACAUUUU | 2597 | AAAAUGUG GGAGGAAACUCC CU UCAAGGACAUCGUCCGGG CCGAGGUG | 8809 |
| 3993 | CACAUUUU G GGAAGUUG | 3630 | CAACUUCC GGAGGAAACUCC CU UCAAGGACAUCGUCCGGG AAAAUGUG | 8810 |
| 3994 | ACAUUUUG G GAAGUUGC | 3631 | GCAACUUC GCAGGAAACUCC CU UCAAGGACAUCGUCCGGG CAAAAUGU | 8811 |
| 3995 | CAUUUUGG G AAGUUGCA | 3632 | UGCAACUU GGAGGAAACUCC CU UCAAGGACAUCGUCCGGG CCAAAAUG | 8812 |
| 3998 | UUUGGGAA G UUGCAUUC | 2598 | GAAUGCAA GGAGGAAACUCC CU UCAAGGACAUCGUCCGGG UUCCCAAA | 8813 |
| 4001 | GCGAAGUU G CAUUCCUU | 2599 | AACGAAUG CGAGGAAACUCC CU UCAAGGACAUCGUCCGGG AACUUCCC | 8814 |
| 4011 | AUUCCUUU G UCUUCAAA | 2600 | UUUGAAGA GGAGGAAACUCC CU UCAACGACAUCGUCCGGG AAAGGAAU | 8815 |
| 4022 | UUCAAACU G UGAAGCAU | 2601 | AUGCUUCA GGAGGAAACUCC CU UCAAGGACAUCGUCCGGG AGUUUGAA | 8816 |
| 4024 | CAAACUGU G AAGCAUUU | 3633 | AAAUCCUU GCAGGAAACUCC CU UCAAGGACAUCGUCCGGG ACAGUUUG | 8817 |
| 4027 | ACUCUCAA G CAUUUACA | 2602 | UCUAAAUC CCAGGAAACUCC CU UCAAGGACAUCGUCCGGG UUCACAGU | 8818 |
| 4036 | CAUUUACA G AAACGCAU | 3634 | AUCCCUUU GGAGGAAACUCC CU UCAAGGACAUCGUCCGGG UGUAAAUG | 8819 |
| 4041 | ACAGAAAC G CAUCCAGC | 2603 | GCUCCAUG GCAGGAAACUCC CU UCAAGGACAUCGUCCGGG GUUUCUGU | 8820 |
| 4048 | CGCAUCCA G CAACAAUA | 2604 | UAUUCUUG GGAGGAAACUCC CU UCAAGGACAUCGUCCGGG UGGAUGCG | 8821 |
| 4052 | UCCAGCAA G AAUAUUGU | 3635 | ACAAUAUU GCAGGAAACUCC CU UCAAGCACAUCGUCCGGG UUGCUGGA | 8822 |
| 4059 | AGAAUAUU G UCCCUUUG | 2605 | CAAAGGGA CCAGGAAACUCC CU UCAAGGACAUCGUCCGGG AAUAUUCU | 8823 |
| 4067 | GUCCCUUU G AGCAGAAA | 3636 | UUUCUGCU GGAGGAAACUCC CU UCAAGGACAUCGUCCGGG AAAGGGAC | 8824 |
| 4069 | CCCUUUGA G CAGAAAUU | 2606 | AAUUUCUG GGAGGAAACUCC CU UCAAGGACAUCGUCCGGG UCAAAGGG | 8825 |
| 4072 | UUUGAGCA G AAAUUUAU | 3637 | AUAAAUUU GGAGGAAACUCC CU UCAAGGACAUCGUCCGGG UGCUCAAA | 8826 |
| 4089 | CUUUCAAA G AGGUAUAU | 3638 | AUAUACCU GGAGGAAACUCC CU UCAAGGACAUCGUCCGGG UUUGAAAG | 8827 |
| 4091 | UUCAAAGA G GUAUAUUU | 3639 | AAAUAUAC GGAGGAAACUCC CU UCAAGGACAUCGUCCGGG UCUUUGAA | 8828 |
| 4092 | UCAAAGAG G UAUAUUUG | 2607 | CAAAUAUA GGAGGAAACUCC CU UCAAGGACAUCGUCCGGG CUCUUUGA | 8829 |
| 4100 | GUAUAUUU G AAAAAAAA | 3640 | UUUUUUUU GGAGGAAACUCC CU UCAAGGACAUCGUCCGCG AAAUAUAC | 8830 |
| 4116 | AAAAAAAA G UAUAUGUG | 2608 | CACAUAUA GGAGGAAACUCC CU UCAAGGACAUCGUCCGGG UUUUUUUU | 8831 |
| 4122 | AACUAUAU G UGAGGAUU | 2609 | AAUCCUCA GGAGGAAACUCC CU UCAAGGACAUCGUCCGGG AUAUACUU | 8832 |
| 4124 | GUAUAUGU G AGGAUUUU | 3641 | AAAAUCCU GGAGGAAACUCC CU UCAAGGACAUCGUCCGGG ACAUAUAC | 8833 |
| 4126 | AUAUGUGA G GAUUUUUA | 3642 | UAAAAAUC GGAGGAAACUCC CU UCAAGGACAUCGUCCGGG UCACAUAU | 8834 |
| 4127 | UAUGUGAG G AUUUUUAU | 3643 | AUAAAAAU GGAGGAAACUCC CU UCAAGGACAUCGUCCGGG CUCACAUA | 8835 |
| 4137 | UUUUUAUU G AUUGGGGA | 3644 | UCCCCAAU GGAGGAAACUCC CU UCAAGGACAUCGUCCGGG AAUAAAAA | 8836 |
| 4141 | UAUUGAUU G GGGAUCUU | 3645 | AAGAUCCC GGAGGAAACUCC CU UCAAGGACAUCGUCCGGG AAUCAAUA | 8837 |
| 4142 | AUUGAUUG G GGAUCUUG | 3646 | CAAGAUCC GGAGGAAACUCC CU UCAAGGACAUCGUCCGGG CAAUCAAU | 8838 |
| 4143 | UUGAUUGG G GAUCUUGG | 3647 | CCAAGACU GGAGGAAACUCC CU UCAAGGACAUCGUCCGGG CCAAUCAA | 8839 |
| 4144 | UGAUUGGG G AUCUUGGA | 3648 | UCCAAGAU GGAGGAAACUCC CU UCAAGGACAUCGUCCGGG CCCAAUCA | 8840 |
| 4150 | GGGAUCUU G GAGUUUUU | 3649 | AAAAACUC GGAGGAAACUCC CU UCAAGGACAUCGUCCGGG AAGAUCCC | 8841 |
| 4151 | GGAUCUUG G AGUUUUUC | 3650 | GAAAAACU GGAGGAAACUCC CU UCAAGGACAUCGUCCGGG CAAGAUCC | 8842 |
| 4153 | AUCUUGGA G UUUUUCAU | 2610 | AUGAAAAA GGAGGAAACUCC CU UCAAGGACAUCGUCCGGG UCCAAGAU | 8843 |
| 4163 | UUUUCAUU G UCGCUAUU | 2611 | AAUAGCGA GGAGGAAACUCC CU UCAAGGACAUCGUCCGGG AAUGAAAA | 8844 |
| 4166 | UCAUUGUC G CUAUUGAU | 2612 | AUCAAUAG GGAGGAAACUCC CU UCAAGGACAUCGUCCGGG GACAAUGA | 8845 |
| 4172 | UCGCUAUU G AUUUUUAC | 3651 | GUAAAAAU GGAGGAAACUCC CU UCAAGGACAUCGUCCGGG AAUAGCGA | 8846 |
| 4187 | ACUUCAAU G GGCUCUUC | 3652 | GAAGAGCC GGAGGAAACUCC CU UCAAGGACAUCGUCCGGG AUUGAAGU | 8847 |
| 4188 | CUUCAAUG G GCUCUUCC | 3653 | GGAAGAGC GGAGGAAACUCC CU UCAAGGACAUCGUCCGGG CAUUGAAG | 8848 |
| 4189 | UUCAAUGG G CUCUUCCA | 2613 | UGGAAGAG GGAGGAAACUCC CU UCAAGGACAUCGUCCGGG CCAUUGAA | 8849 |
| 4202 | UCCAACAA G GAAGAAGC | 3654 | GCUUCUUC GGAGGAAACUCC CU UCAAGGACAUCGUCCGGG UUGUUGGA | 8850 |
| 4203 | CCAACAAG G AAGAAGCU | 3655 | AGCUUCUU GGAGGAAACUCC CU UCAAGGACAUCGUCCGGG CUUGUUGG | 8851 |
| 4206 | ACAAGGAA G AAGCUUGC | 3656 | GCAAGCUU GGAGGAAACUCC CU UCAAGGACAUCGUCCGGG UUCCUUGU | 8852 |
| 4209 | AGGAAGAA G CUUGCUGG | 2614 | CCAGCAAG GGAGGAAACUCC CU UCAAGGACAUCGUCCGGG UUCUUCCU | 8853 |
| 4213 | AGAAGCUU G CUGGUAGC | 2615 | GCUACCAG GGAGGAAACUCC CU UCAAGGACAUCGUCCGGG AAGCUUCU | 8854 |
| 4216 | AGCUUGCU G GUAGCACU | 3657 | AGUGCUAC GGAGGAAACUCC CU UCAAGGACAUCGUCCGGG AGCAAGCU | 8855 |
| 4217 | GCUUGCUG G UAGCACUU | 2616 | AAGUGCUA GGAGGAAACUCC CU UCAAGGACAUCGUCCGGG CAGCAAGC | 8856 |
| 4220 | UGCUGGUA G CACUUGCU | 2617 | AGCAAGUG GGAGGAAACUCC CU UCAAGGACAUCGUCCGGG UACCAGCA | 8857 |
| 4226 | UAGCACUU G CUACCCUG | 2618 | CAGGGUAG GGAGGAAACUCC CU UCAAGGACAUCGUCCGGG AAGUGCUA | 8858 |
| 4234 | GCUACCCU G AGUUCAUC | 3658 | GAUGAACU GGAGGAAACUCC CU UCAAGGACAUCGUCCGGG AGGGUAGC | 8859 |
| 4236 | UACCCUGA G UUCAUCCA | 2619 | UGGAUGAA GGAGGAAACUCC CU UCAAGGACAUCGUCCGGG UCAGGGUA | 8860 |
| 4245 | UUCAUCCA G GCCCAACU | 3659 | AGUUGGGC GGAGGAAACUCC CU UCAAGGACAUCGUCCGGG UGGAUGAA | 8861 |
| 4246 | UCAUCCAG G CCCAACUG | 2620 | CAGUUGGG GGAGGAAACUCC CU UCAAGGACAUCGUCCGGG CUGGAUGA | 8862 |
| 4254 | GCCCAACU G UGAGCAAG | 2621 | CUUGCUCA GGAGGAAACUCC CU UCAAGGACAUCGUCCGGG AGUUGGGC | 8863 |
| 4256 | CCAACUGU G AGCAAGGA | 3660 | UCCUUGCU GGAGGAAACUCC CU UCAAGGACAUCGUCCGGG ACAGUUGG | 8864 |
| 4258 | AACUGUGA G CAAGGAGC | 2622 | GCUCCUUG GGAGGAAACUCC CU UCAAGGACAUCGUCCGGG UCACAGUU | 8864 |
| 4262 | GUGAGCAA G GAGCACAA | 3661 | UUGUGCUC GGAGGAAACUCC CU UCAAGGACAUCGUCCGGG UUGCUCAC | 8866 |
| 4263 | UGAGCAAG G AGCACAAG | 3662 | CUUGUGCU GGAGGAAACUCC CU UCAAGGACAUCGUCCGGG CUUGCUCA | 8867 |
| 4265 | AGCAAGGA G CACAAGCC | 2623 | GGCUUGUG GGAGGAAACUCC CU UCAAGGACAUCGUCCGGG UCCUUGCU | 8868 |
| 4271 | GAGCACAA G CCACAAGU | 2624 | ACUUGUGG GGAGGAAACUCC CU UCAAGGACAUCGUCCGGG UUGUGCUC | 8869 |
| 4278 | AGCCACAA G UCUUCCAG | 2625 | CUGGAAGA GGAGGAAACUCC CU UCAAGGACAUCGUCCGGG UUGUGGCU | 8870 |
| 4286 | GUCUUCCA G AGGAUGCU | 3663 | AGCAUCCU GGAGGAAACUCC CU UCAAGGACAUCGUCCGGG UGGAAGAC | 8871 |
| 4288 | CUUCCAGA G GAUGCUUG | 3664 | CAAGCAUC GGAGGAAACUCC CU UCAAGGACAUCGUCCGGG UCUGGAAG | 8871 |
| 4289 | UUCCAGAG G AUGCUUGA | 3665 | UCAAGCAU GGAGGAAACUCC CU UCAAGGACAUCGUCCGGG CUCUGGAA | 8873 |
| 4292 | CAGAGGAU G CUUGAUUC | 2926 | GAAUCAAG GGAGGAAACUCC CU UCAAGGACAUCGUCCGGG AUCCUCUG | 8874 |
| 4296 | GCAUGCUU G AUUCCAGU | 3666 | ACUGGAAU GGAGGAAACUCC CU UCAAGGACAUCGUCCGGG AAGCAUCC | 8875 |
| 4303 | UGAUUCCA G UGGUUCUG | 2627 | CAGAACCA GGAGGAAACUCC CU UCAAGGACAUCGUCCGGG UGGAAUCA | 8876 |
| 4305 | AUUCCAGU G GUUCUGCU | 3667 | AGCAGAAC GGAGGAAACUCC CU UCAAGGACAUCGUCCGGG ACUGGAAU | 8877 |
| 4306 | UUCCAGUG G UUCUGCUU | 2628 | AAGCAGAA GGAGGAAACUCC CU UCAAGGACAUCGUCCGGG CACUGGAA | 8878 |
| 4311 | GUGGUUCU G CUUCAAGG | 2629 | CCUUGAAG GGAGGAAACUCC CU UCAAGGACAUCGUCCGGG AGAACCAC | 8879 |
| 4318 | UGCUUCAA G GCUUCCAC | 3668 | GUGGAAGC GGAGGAAACUCC CU UCAAGGACAUCGUCCGGG UUGAAGCA | 8880 |
| 4319 | GCUUCAAG G CUUCCACU | 2360 | AGUGGAAG GGAGGAAACUCC CU UCAAGGACAUCGUCCGGG CUUGAAGC | 8881 |
| 4328 | CUUCCACU G CAAAACAC | 2631 | GUGUUUUG GGAGGAAACUCC CU UCAAGGACAUCGUCCGGG AGUGGAAG | 8882 |
| 4341 | ACACUAAA G AUCCAAGA | 3669 | UCUUGGAU GGAGGAAACUCC CU UCAAGGACAUCGUCCGGG UUUAGUGU | 8883 |
| 4348 | AGAUCCAA G AAGGCCUU | 3670 | AAGGCCUU GGAGGAAACUCC CU UCAAGGACAUCGUCCGGG UUGGAUCU | 8884 |
| 4351 | UCCAAGAA G GCCUUCAU | 3671 | AUGAAGGC GGAGGAAACUCC CU UCAAGGACAUCGUCCGGG UUCUUGGA | 8885 |
| 4352 | CCAAGAAG G CCUUCAUG | 2632 | CAUGAAGG GGAGGAAACUCC CU UCAAGGACAUCGUCCGGG CUUCUUGG | 8886 |
| 4360 | GCCUUCAU G GCCCCAGC | 3672 | GCUGGGGC GGAGGAAACUCC CU UCAAGGACAUCGUCCGGG AUGAAGGC | 8887 |
| 4361 | CCUUCAUG G CCCCAGCA | 2633 | UGCUGGGG GGAGGAAACUCC CU UCAAGGACAUCGUCCGGG CAUGAAGG | 8888 |
| 4367 | UGGCCCCA G CAGGCCGG | 2634 | CCGGCCUG GGAGGAAACUCC CU UCAAGGACAUCGUCCGGG UGGGGCCA | 8889 |
| 4370 | CCCCAGCA G GCCGGAUC | 3673 | GAUCCGGC GGAGGAAACUCC CU UCAAGGACAUCGUCCGGG UGCUGGGG | 8890 |
| 4371 | CCCAGCAG G CCGGAUCG | 2635 | CGAUCCGG GGAGGAAACUCC CU UCAAGGACAUCGUCCGGG CUGCUGGG | 8891 |
| 4374 | AGCAGGCC G GAUCGGUA | 3674 | UACCGAUC GGAGGAAACUCC CU UCAAGGACAUCGUCCGGG GGCCUGCU | 8892 |
| 4375 | GCAGGCCG G AUCGGUAC | 3675 | GUACCGAU GGAGGAAACUCC CU UCAAGGACAUCGUCCGGG CGGCCUGC | 8893 |
| 4379 | GCCGGAUC G GUACUGUA | 3676 | UACAGUAC GGAGGAAACUCC CU UCAAGGACAUCGUCCGGG GAUCCGGC | 8894 |
| 4380 | CCGGAUCG G UACUGUAU | 2636 | AUACAGUA GGAGGAAACUCC CU UCAAGGACAUCGUCCGGG CGAUCCGG | 8895 |
| 4385 | UCGGUACU G UAUCAAGU | 2637 | ACUUGAUA GGAGGAAACUCC CU UCAAGGACAUCGUCCGGG AGUACCGA | 8896 |
| 4392 | UGUAUCAA G UCAUGGCA | 2638 | UGCCAUGA GGAGGAAACUCC CU UCAAGGACAUCGUCCGGG UUGAUACA | 8897 |
| 4397 | CAAGUCAU G GCAGGUAC | 3677 | GUACCUGC GGAGGAAACUCC CU UCAAGGACAUCGUCCGGG AUGACUUG | 8898 |
| 4398 | AAGUCAUG G CAGGUACA | 2639 | UGUACCUG GGAGGAAACUCC CU UCAAGGACAUCGUCCGGG CAUGACUU | 8899 |
| 4401 | UCAUGGCA G GUACAGUA | 3678 | UACUGUAC GGAGGAAACUCC CU UCAAGGACAUCGUCCGGG UGCCAUGA | 8900 |
| 4402 | CAUGGCAG G UACAGUAG | 2640 | CUACUGUA GGAGGAAACUCC CU UCAAGGACAUCGUCCGGG CUGCCAUG | 8901 |
| 4407 | CAGGUACA G UAGGAUAA | 2641 | UUAUCCUA GGAGGAAACUCC CU UCAAGGACAUCGUCCGGG UGUACCUG | 8902 |
| 4410 | GUACAGUA G GAUAAGCC | 3679 | GGCUUAUC GGAGGAAACUCC CU UCAAGGACAUCGUCCGGG UACUGUAC | 8903 |
| 4411 | UACAGUAG G AUAAGCCA | 3680 | UGGCUUAU GGAGGAAACUCC CU UCAAGGACAUCGUCCGGG CUACUGUA | 8904 |
| 4416 | UAGGAUAA G CCACUCUG | 2642 | CAGAGUGG GGAGGAAACUCC CU UCAAGGACAUCGUCCGGG UUAUCCUA | 8905 |
| 4424 | GCCACUCU G UCCCUUCC | 2643 | GGAAGGGA GGAGGAAACUCC CU UCAAGGACAUCCUCCGGG AGGAAGGG | 8906 |
| 4434 | CCCUUCCU G GGCAAAGA | 3681 | UCUUUGCC GGAGGAAACUCC CU UCAAGGACAUCGUCCGGG AGGAAGGG | 8907 |
| 4435 | CCUUCCUG G GCAAAGAA | 3682 | UUCUUUGC GGAGGAAACUCC CU UCAAGGACAUCGUCCGGG CAGGAAGG | 8908 |
| 4436 | CUUCCUGG G CAAAGAAG | 2644 | CUUCUUUG GGAGGAAACUCC CU UCAAGGACAUCGUCCGGG CCAGGAAG | 8909 |
| 4441 | UGGGCAAA G AAGAAACG | 3683 | CGUUUCUU GGAGGAAACUCC CU UCAAGGACAUCGUCCGGG UUUGCCCA | 8910 |
| 4444 | GCAAAGAA G AAACGGAG | 3684 | CUCCGUUU GGAGGAAACUCC CU UCAAGGACAUCGUCCGGG UUCUUUGC | 8911 |
| 4419 | GAAGAAAC G GAGGGGAU | 3685 | AUCCCCUC GGAGGAAACUCC CU UCAAGGACAUCGUCCGGG GUUUCUUC | 8912 |
| 4450 | AAGAAACG G AGGGGAUG | 3687 | CAUCCCCU GGAGGAAACUCC CU UCAAGGACAUCGUCCGGG CGUUUCUU | 8913 |
| 4452 | GAAACGGA G GGGAUGAA | 3687 | UUCAUCCC GGAGGAAACUCC CU UCAAGGACAUCGUCCGGG UCCGUUUC | 8914 |
| 4453 | AAACGGAG G GGAUGAAU | 3688 | AUUCAUCC GGAGGAAACUCC CU UCAAGGACAUCGUCCGGG CUCCGUUU | 8915 |
| 4454 | AACGGAGG G GAUGAAUU | 3689 | AAUUCAUC GGAGGAAACUCC CU UCAAGGACAUCGUCCGGG CCUCCGUU | 8916 |
| 4455 | ACGGAGGG G AUGAAUUC | 3690 | GAAUUCAU GGAGGAAACUCC CU UCAAGGACAUCGUCCGGG CCCUCCGU | 8917 |
| 4458 | GAGGGGAU G AAUUCUUC | 3691 | GAAGAAUU GGAGGAAACUCC CU UCAAGGACAUCGUCCGGG AUCCCCUC | 8918 |
| 4471 | CUUCCUUA G ACUUACUU | 3692 | AAGUAAGU GGAGGAAACUCC CU UCAAGGACAUCGUCCGGG UAAGGAAG | 8919 |
| 4482 | UUACUUUU G UAAAAAUG | 2645 | CAUUUUUA GGAGGAAACUCC CU UCAAGGACAUCGUCCGGG AAAAGUAA | 8920 |
| 4490 | GUAAAAAU G UCCCCACG | 2646 | CGUGGGGA GGAGGAAACUCC CU UCAAGGACAUCGUCCGGG AUUUUUAC | 8921 |
| 4498 | GUCCCCAC G GUACUUAC | 3693 | GUAAGUAC GGAGGAAACUCC CU UCAAGGACAUCGUCCGGG GUGGGGAC | 8922 |
| 4499 | UCCCCACG G UACUUACU | 2647 | AGUAAGUA GGAGGAAACUCC CU UCAAGGACAUCGUCCGGG CGUGGGGA | 8923 |
| 4515 | UCCCCACU G AUGGACCA | 3694 | UGGUCCAU GGAGGAAACUCC CU UCAAGGACAUCGUCCGGG AGUGGGGA | 8924 |
| 4518 | CCACUGAU G GACCAGUG | 3695 | CACUGGUC GGAGGAAACUCC CU UCAAGGACAUCGUCCGGG AUCAGUGG | 8925 |
| 4519 | CACUGAUG G ACCAGUGG | 3696 | CCACUGGU GGAGGAAACUCC CU UCAAGGACAUCGUCCGGG CAUCAGUG | 8926 |
| 4524 | AUGGACCA G UGGUUUCC | 2648 | GGAAACCA GGAGGAAACUCC CU UCAAGGACAUCGUCCGGG UGGUCCAU | 8927 |
| 4526 | GGACCAGU G GUUUCCAG | 3697 | CUGGAAAC GGAGGAAACUCC CU UCAAGGACAUCGUCCGGG ACUGGUCC | 8928 |
| 4527 | GACCAGUG G UUUCCAGU | 2649 | ACUGGAAA GGAGGAAACUCC CU UCAAGGACAUCGUCCGGG CACUGGUC | 8929 |
| 4534 | GGUUUCCA G UCAUGAGC | 2650 | GCUCAUGA GGAGGAAACUCC CU UCAAGGACAUCGUCCGGG UGGAAACC | 8930 |
| 4539 | CCAGUCAU G AGCGUUAG | 3698 | CUAACGCU GGAGGAAACUCC CU UCAAGGACAUCGUCCGGG AUGACUGG | 8931 |
| 4541 | AGUCAUGA G CGUUAGAC | 2651 | GUCUAACG GGAGGAAACUCC CU UCAAGGACAUCGUCCCGG UCAUGACU | 8932 |
| 4543 | UCAUGAGC G UUAGACUG | 2652 | CAGUCUAA GGAGGAAACUCC CU UCAAGGACAUCGUCCGGG GCUCAUGA | 8933 |
| 4547 | GAGCGUUA G ACUGACUU | 3699 | AAGUCAGU GGAGGAAACUCC CU UCAAGGACAUCGUCCGGG UAACGCUC | 8934 |
| 4551 | GUUAGACU G ACUUGUUU | 3700 | AAACAAGU GGAGGAAACUCC CU UCAAGGACAUCGUCCGGG AGUCUAAC | 8935 |
| 4556 | ACUGACUU G UUUGUCUU | 2653 | AAGACAAA GGAGGAAACUCC CU UCAAGGACAUCGUCCGGG AAGUCAGU | 8936 |
| 4560 | ACUUGUUU G UCUUCCAU | 2654 | AUGGAAGA GGAGGAAACUCC CU UCAAGGACAUCGUCCGGG AAACAAGU | 8937 |
| 4575 | AUUCCAUU G UUUUGAAA | 2655 | UUUCAAAA GGAGGAAACUCC CU UCAAGGACAUCGUCCGGG AAUGGAAU | 8938 |
| 4580 | AUUGUUUU G AAACUCAG | 3701 | CUGAGUUU GGAGGAAACUCC CU UCAAGGACAUCGUCCGGG AAAACAAU | 8939 |
| 4588 | GAAACUCA G UAUGCCGC | 2656 | GCGGCAUA GGACGAAACUCC CU UCAAGGACAUCGUCCCGG UGAGUUUC | 8940 |
| 4592 | CUCAGUAU G CCGCCCCU | 2657 | AGGGGCGG GGAGGAAACUCC CU UCAAGGACAUCGUCCGGG AUACUGAG | 8941 |
| 4595 | AGUAUGCC G CCCCUGUC | 2658 | GACAGGGG GGAGGAAACUCC CU UCAAGGACAUCGUCCGGG GGCAUACU | 8942 |
| 4601 | CCGCCCCU G UCUUGCUG | 2659 | CAGCAAGA GGAGGAAACUCC CU UCAAGGACAUCGUCCGGG AGGGGCGG | 8943 |
| 4606 | CCUGUCUU G CUGUCAUG | 2660 | CAUGACAG GGAGGAAACUCC CU UCAAGGACAUCGUCCGGG AAGACAGG | 8944 |
| 4609 | GCUGUCAU G UCAUGAAA | 2661 | UUUCAUGA GGAGGAAACUCC CU UCAAGGACAUCGUCCGGG AGCAAGAC | 8945 |
| 4614 | GCUGUCAU G AAAUCAGC | 3702 | GCUGAUUU GGAGGAAACUCC CU UCAAGGACAUCGUCCGGG AUGACAGC | 8946 |
| 4621 | UGAAAUCA G CAAGAGAG | 2662 | CUCUCUUG GGAGGAAACUCC CU UCAAGGACAUCGUCCGGG UGAUUUCA | 8947 |
| 4625 | AUCAGCAA G AGAGGAUG | 3703 | CAUCCUCU GGAGGAAACUCC CU UCAAGGACAUCGUCCGGG UUGCUGAU | 8948 |
| 4627 | CAGCAAGA G AGGAUGAC | 3704 | GUCAUCCU GGAGGAAACUCC CU UCAAGGACAUCGUCCGGG UCUUGCUG | 8949 |
| 4629 | GCAAGAGA G GAUGACAC | 3705 | GUGUCAUC GGAGGAAACUCC CU UCAAGGACAUCGUCCGGG UCUCUUGC | 8950 |
| 4630 | CAAGAGAG G AUGACACA | 3706 | UGUGUCAU GGAGGAAACUCC CU UCAAGGACAUCGUCCGGG CUCUCUUG | 8951 |
| 4633 | GAGAGGAU G ACACAUCA | 3707 | UGAUGUGU GGAGGAAACUCC CU UCAAGGACAUCGUCCGGG AUCCUCUC | 8952 |
| 4653 | AAUAACUC G GAUUCCAG | 3708 | CUGGAAUC GGAGGAAACUCC CU UCAAGGACAUCGUCCGGG GAGUUAUU | 8953 |
| 4654 | AUAACUCG G AUUCCAGC | 3709 | GCUGGAAU GGAGGAAACUCC CU UCAAGGACAUCGUCCGGG CGAGUUAU | 8954 |
| 4661 | GGAUUCCA G CCCACAUU | 2663 | AAUGUGGG GGAGGAAACUCC CU UCAAGGACAUCGUCCGGG UGGAAUCC | 8955 |
| 4670 | CCCACAUU G GAUUCAUC | 3170 | GAUGAUUC GGAGGAAACUCC CU UCAAGGACAUCGUCCGGG AAUGUGGG | 8956 |
| 4671 | CCACAUUG G AUUCAUCA | 3711 | UGAUGAAU GGAGGAAACUCC CU UCAAGGACAUCGUCCGGG CAAUGUGG | 8957 |
| 4680 | AUUCAUCA G CAUUUGGA | 2664 | UCCAAAUG GGAGGAAACUCC CU UCAAGGACAUCGUCCGGG UGAUGAAU | 8958 |
| 4686 | CAGCAUUU G GACCAAUA | 3712 | UAUUGGUC GGAGGAAACUCC CU UCAAGGACAUCGUCCGGG AAAUGCUG | 8959 |
| 4687 | AGCAUUUG G ACCAAUAG | 3713 | CUAUUGGU GGAGGAAACUCC CU UCAAGGACAUCGUCCGGG CAAAUGCU | 8960 |
| 4695 | GACCAAUA G CCCACAGC | 2665 | GCUGUGGG GGAGGAAACUCC CU UCAAGGACAUCGUCCGGG UAUUGGUC | 8961 |
| 4702 | AGCCCACA G CUGAGAAU | 2666 | AUUCUCAG GGAGGAAACUCC CU UCAAGGACAUCGUCCGGG UGUGGGCU | 8962 |
| 4705 | CCACAGCU G AGAAUGUG | 3714 | CACAUUCU GGAGGAAACUCC CU UCAAGGACAUCGUCCGGG AGCUGUGG | 8963 |
| 4707 | ACAGCUGA G AAUGUGGA | 3715 | UCCACAUU GGAGGAAACUCC CU UCAAGGACAUCGUCCGGG UCAGCUGU | 8964 |
| 4711 | CUGAGAAU G UGGAAUAC | 2667 | GUAUUCCA GGAGGAAACUCC CU UCAAGGACAUCGUCCGGG AUUCUCAG | 8965 |
| 4713 | GAGAAUGU G GAAUACCU | 3716 | AGGUAUUC GGAGGAAACUCC CU UCAAGGACAUCGUCCGGG ACAUUCUC | 8966 |
| 4714 | AGAAUGUG G AAUACCUA | 3717 | UAGGUAUU GGAGGAAACUCC CU UCAAGGACAUCGUCCGGG CACAUUCU | 8967 |
| 4724 | AUACCUAA G GAUAACAC | 3718 | GUGUUAUC GGAGGAAACUCC CU UCAAGGACAUCGUCCGGG UUAGGUAU | 8968 |
| 4725 | UACCUAAG G AUAACACC | 3719 | GGUGUUAU GGAGGAAACUCC CU UCAAGGACAUCGUCCGGG CUUAGGUA | 8969 |
| 4734 | AUAACACC G CUUUUGUU | 2668 | AACAAAAG GGAGGAAACUCC CU UCAAGGACAUCGUCCGGG GGUGUUAU | 8970 |
| 4740 | CCGCUUUU G UUCUCGCA | 2669 | UGCGAGAA GGAGGAAACUCC CU UCAAGGACAUCGUCCGGG AAAAGCGG | 8971 |
| 4746 | UUGUUCUC G CAAAAACG | 2670 | CGUUUUUG GGAGGAAACUCC CU UCAAGGACAUCGUCCGGG GAGAACAA | 8972 |
| 4754 | GCAAAAAC G UAUCUCCU | 2671 | AGGAGAUA GGAGGAAACUCC CU UCAAGGACAUCGUCCGGG GUUUUUGC | 8973 |
| 4768 | CCUAAUUU G AGGCUCAG | 3720 | CUGAGCCU GGAGGAAACUCC CU UCAAGGACAUCGUCCGGG AAAUUAGG | 8974 |
| 4770 | UAAUUUGA G GCUCAGAU | 3721 | AUCUGAGC GGAGGAAACUCC CU UCAAGGACAUCGUCCGGG UCAAAUUA | 8975 |
| 4771 | AAUUUGAG G CUCAGAUG | 2672 | CAUCUGAG GGAGGAAACUCC CU UCAAGGACAUCGUCCGGG CUCAAAUU | 8976 |
| 4776 | GAGGCUCA G AUGAAAUG | 3722 | CAUUUCAU GGAGGAAACUCC CU UCAAGGACAUCGUCCGGG UGAGCCUC | 8977 |
| 4779 | GCUCAGAU G AAAUGCAU | 3723 | AUGCAUUU GGAGGAAACUCC CU UCAAGGACAUCGUCCGGG AUCUGAGC | 8978 |
| 4784 | GAUGAAAU G CAUCAGGU | 2673 | ACCUGAUG GGAGGAAACUCC CU UCAAGGACAUCGUCCGGG AUUUCAUC | 8979 |
| 4790 | AUGCAUCA G GUCCUUUG | 3724 | CAAAGGAC GGAGGAAACUCC CU UCAAGGACAUCGUCCGGG UGAUGCAU | 8980 |
| 4791 | UGCAUCAG G UCCUUUGG | 2674 | CCAAAGGA GGAGGAAACUCC CU UCAAGGACAUCGUCCGGG CUGAUGCA | 8981 |
| 4798 | GGUCCUUU G GGGCAUAG | 3725 | CUAUGCCC GGAGGAAACUCC CU UCAAGGACAUCGUCCGGG AAAGGACC | 8982 |
| 4799 | GUCCUUUG G GGCAUAGA | 3726 | UCUAUGCC GGAGGAAACUCC CU UCAAGGACAUCGUCCGGG CAAAGGAC | 8983 |
| 4800 | UCCUUUGG G GCAUAGAU | 3727 | AUCUAUGC GGAGGAAACUCC CU UCAAGGACAUCGUCCGGG CCAAAGGA | 8984 |
| 4801 | CCUUUGGG G CAUAGAUC | 2675 | GAUCUAUG GGAGGAAACUCC CU UCAAGGACAUCGUCCGGG CCCAAAGG | 8985 |
| 4806 | GGGGCAUG G AUCAGAAG | 3728 | CUUCUGUA GGAGGAAACUCC CU UCAAGGACAUCGUCCGGG UAUGCCCC | 8986 |
| 4811 | AUAGAUCA G AAGACUAC | 3729 | GUAGUCUU GGAGGAAACUCC CU UCAAGGACAUCGUCCGGG UGAUCUAU | 8987 |
| 4814 | GAUCAGAA G ACUACAAA | 3730 | UUUGUAGU GGAGGAAACUCC CU UCAAGGACAUCGUCCGGG UUCUGAUC | 8988 |
| 4826 | ACAAAAAU G AAGCUGCU | 3731 | AGCAGCUU GGAGGAAACUCC CU UCAAGGACAUCGUCCGGG AUUUUUGU | 8989 |
| 4829 | AAAAUGAA G CUGCUCUG | 2676 | CAGAGCAG GGAGGAAACUCC CU UCAAGGACAUCGUCCGGG UUCAUUUU | 8990 |
| 4832 | AUGAAGCU G CUCUGAAA | 2677 | UUUCAGAG GGAGGAAACUCC CU UCAAGGACAUCGUCCGGG AGCUUCAU | 8991 |
| 4837 | GCUGCUCU G AAAUCUCC | 3732 | GGAGAUUU GGAGGAAACUCC CU UCAAGGACAUCGUCCGGG AGAGCAGC | 8992 |
| 4850 | CUCCUUUA G CCAUCACC | 2678 | GGUGAUGG GGAGGAAACUCC CU UCAAGGACAUCGUCCGGG UAAAGGAG | 8993 |
| 4876 | CAAAAUUA G UUUGUGUU | 2679 | AACACAAA GGAGGAAACUCC CU UCAAGGACAUCGUCCGGG UAAUUUUG | 8994 |
| 4880 | AUUAGUUU G UGUUACUU | 2680 | AAGUAACA GGAGGAAACUCC CU UCAAGGACAUCGUCCGGG AAACUAAU | 8995 |
| 4882 | UAGUUUGU G UUACUUAU | 2681 | AUAAGUAA GGAGGAAACUCC CU UCAAGGACAUCGUCCGGG ACAAACUA | 8996 |
| 4891 | UUACUUAU G GAAGAUAG | 3733 | CUAUCUUC GGAGGAAACUCC CU UCAAGGACAUCGUCCGGG AUAAGUAA | 8997 |
| 4892 | UACUUAUG G AAGAUAGU | 3734 | ACUAUCUU GGAGGAAACUCC CU UCAAGGACAUCGUCCGGG CAUAAGUA | 8998 |
| 4895 | UUAUGGAA G AUAGUUUU | 3735 | AAAACUAU GGAGGAAACUCC CU UCAAGGACAUCGUCCGGG UUCCAUAA | 8999 |
| 4899 | GGAAGAUA G UUUUCUCC | 2682 | GGAGAAAA GGAGGAAACUCC CU UCAAGGACAUCGUCCGGG UAUCUUCC | 9000 |
| 4926 | CUUCAAAA G CUUUUUAC | 2683 | GUAAAAAG GGAGGAAACUCC CU UCAAGGACAUCGUCCGGG UUUUGAAG | 9001 |
| 4940 | UACUCAAA G ACUAUAUG | 3736 | CAUAUACU GGAGGAAACUCC CU UCAAGGACAUCGUCCGGG UUUGAGUA | 9002 |
| 4942 | CUCAAAGA G UAUAUGUU | 2684 | AACAUAUA GGAGGAAACUCC CU UCAAGGACAUCGUCCGGG UCUUUGAG | 9003 |
| 4948 | GAGUAUAU G UUCCCUCC | 2685 | GGAGGGAA GGAGGAAACUCC CU UCAAGGACAUCGUCCGGG AUAUACUC | 9004 |
| 4958 | UCCCUCCA G GUCAGCUG | 3737 | CACCUGAC GGAGGAAACUCC CU UCAAGGACAUCGUCCGGG UGGAGGGA | 9005 |
| 4959 | CCCUCCAG G UCAGCUGC | 2686 | GCAGCUGA GGAGGAAACUCC CU UCAAGGACAUCGUCCGGG CUGGAGGG | 9006 |
| 4963 | CCAGGUCA G CUGCCCCC | 2687 | GGGGGCAG GGAGGAAACUCC CU UCAAGGACAUCGUCCGGG UGACCUGG | 9007 |
| 4966 | GGUCAGCU G CCCCCAAA | 2688 | UUUGGGGG GGAGGAAACUCC CU UCAAGGACAUCGUCCGGG AGCUGACC | 9008 |
| 4987 | CUCCUUAC G CUUUGUCA | 2689 | UGACAAAG GGAGGAAACUCC CU UCAAGGACAUCGUCCGGG GUAAGGAG | 9009 |
| 4992 | UACGCUUU G UCACACAA | 2690 | UUGUGUGA GGAGGAAACUCC CU UCAAGGACAUCGUCCGGG AAAGCGUA | 9010 |
| 5004 | CACAAAAA G UGUCUCUG | 2691 | CAGAGACA GGAGGAAACUCC CU UCAAGGACAUCGUCCGGG UUUUUGUG | 9011 |
| 5006 | CAAAAAGU G UCUCUGCC | 2692 | GGCAGAGA GGAGGAAACUCC CU UCAAGGACAUCGUCCGGG ACUUUUUG | 9012 |
| 5012 | GUGUCUCU G CCUUGAGU | 2693 | ACUCAAGG GGAGGAAACUCC CU UCAAGGACAUCGUCCGGG AGAGACAC | 9013 |
| 5017 | UCUGCCUU G AGUCAUCU | 3738 | AGAUGACU GGAGGAAACUCC CU UCAAGGACAUCGUCCGGG AAGGCAGA | 9014 |
| 5019 | UGCCUUGA G UCAUCUAU | 2694 | AUAGAUGA GGAGGAAACUCC CU UCAAGGACAUCGUCCGGG UCAAGGCA | 9015 |
| 5032 | CUAUUCAA G CACUUACA | 2695 | UGUAAGUG GGAGGAAACUCC CU UCAAGGACAUCGUCCGGG UUGAAUAG | 9016 |
| 5041 | CACUUACA G CUCUGGCC | 2696 | GGCCAGAG GGAGGAAACUCC CU UCAAGGACAUCGUCCGGG UGUAAGUG | 9017 |
| 5046 | ACAGCUCU G GCCACAAC | 3739 | GUUGUGGC GGAGGAAACUCC CU UCAAGGACAUCGUCCGGG AGAGCUGU | 9018 |
| 5047 | CAGCUCUG G CCACAACA | 2697 | UGUUGUGG GGAGGAAACUCC CU UCAAGGACAUCGUCCGGG CAGAGCUG | 9019 |
| 5056 | CCACAACA G GGCAUUUU | 3740 | AAAAUGCC GGAGGAAACUCC CU UCAAGGACAUCGUCCGGG UGUUGUGG | 9020 |
| 5057 | CACAACAG G GCAUUUUA | 3741 | UAAAAUGC GGAGGAAACUCC CU UCAAGGACAUCGUCCGGG CUGUUGUG | 9021 |
| 5058 | ACAACAGG G CAUUUUAC | 2698 | GUAAAAUG GGAGGAAACUCC CU UCAAGGACAUCGUCCGGG CCUGUUGU | 9022 |
| 5068 | AUUUUACA G GUGCGAAU | 3742 | AUUCGCAC GGAGGAAACUCC CU UCAAGGACAUCGUCCGGG UGUAAAAU | 9023 |
| 5069 | UUUUACAG G UGCGAAUG | 2699 | CAUUCGCA GGAGGAAACUCC CU UCAAGGACAUCGUCCGGG CUGUAAAA | 9024 |
| 5071 | UUACAGGU G CGAAUGAC | 2700 | GUCAUUCG GGAGGAAACUCC CU UCAAGGACAUCGUCCGGG ACCUGUAA | 9025 |
| 5073 | ACAGGUGC G AAUGACAG | 3743 | CUGUCAUU GGAGGAAACUCC CU UCAAGGACAUCGUCCGGG GCACCUGU | 9026 |
| 5077 | GUGCGAAU G ACAGUAGC | 3744 | GCUACUGU GGAGGAAACUCC CU UCAAGGACAUCGUCCGGG AUUCGCAC | 9027 |
| 5081 | GAAUGACA G UAGCAUUA | 2701 | UAAUGCUA GGAGGAAACUCC CU UCAAGGACAUCGUCCGGG UGUCAUUC | 9028 |
| 5084 | UGACAGUA G CAUUAUGA | 2702 | UCAUAAUG GGAGGAAACUCC CU UCAAGGACAUCGUCCGGG UACUGUCA | 9029 |
| 5091 | AGCAUUAU G AGUAGUGU | 3745 | ACACUACU GGAGGAAACUCC CU UCAAGGACAUCGUCCGGG AUAAUGCU | 9030 |
| 5093 | CAUUAUGA G UAGUGUGA | 2703 | UCACACUA GGAGGAAACUCC CU UCAAGGACAUCGUCCGGG UCAUAAUC | 9031 |
| 5096 | UAUGAGUA G UGUGAAUU | 2704 | AAUUCACA GGAGGAAACUCC CU UCAAGGACAUCGUCCGGG UACUCAUA | 9032 |
| 5098 | UGAGUAGU G UGAAUUCA | 2705 | UGAAUUCA GGAGGAAACUCC CU UCAAGGACAUCGUCCGGG ACUACUCA | 9033 |
| 5100 | AGUAGUGU G AAUUCAGG | 3746 | CCUGAAUU GGAGGAAACUCC CU UCAAGGACAUCGUCCGGG ACACUACU | 9034 |
| 5107 | UGAAUUCA G GUAGUAAA | 3747 | UUUACUAC GGAGGAAACUCC CU UCAAGGACAUCGUCCGGG UGAAUUCA | 9035 |
| 5108 | GAAUUCAG G UAGUAAAU | 2706 | AUUUACUA GGAGGAAACUCC CU UCAAGGACAUCGUCCGGG CUGAAUUC | 9036 |
| 5111 | UUCAGGUA G UAAAUAUG | 2707 | CAUAUUUA GGAGGAAACUCC CU UCAAGGACAUCGUCCGGG UACCUGAA | 9037 |
| 5119 | GUAAAUAU G AAACUAGG | 3748 | CCUAGUUU GGAGGAAACUCC CU UCAAGGACAUCGUCCGGG AUAUUUAC | 9038 |
| 5126 | UGAAACUA G GGUUUGAA | 3749 | UUCAAACC GGAGGAAACUCC CU UCAAGGACAUCGUCCGGG UAGUUUCA | 9039 |
| 5127 | GAAACUAG G GUUUGAAA | 3750 | UUUCAAAC GGAGGAAACUCC CU UCAAGGACAUCGUCCGGG CUAGUUUC | 9040 |
| 5128 | AAACUAGG G UUUGAAAU | 2708 | AUUUCAAA GGAGGAAACUCC CU UCAAGGACAUCGUCCGGG CCUAGUUU | 9041 |
| 5132 | UAGGGUUU G AAAUUGAU | 3751 | AUCAAUUU GGAGGAAACUCC CU UCAAGGACAUCGUCCGGG AAACCCUA | 9042 |
| 5138 | UUGAAAUU G AUAAUGCU | 3752 | AGCAUUAU GGAGGAAACUCC CU UCAAGGACAUCGUCCGGG AAUUUCAA | 9043 |
| 5144 | UUGAUAAU G CUUUCACA | 2709 | UGUGAAAG GGAGGAAACUCC CU UCAAGGACAUCGUCCGGG AUUAUCAA | 9044 |
| 5159 | CAACAUUU G CAGAUGUU | 2710 | AACAUCUG GGAGGAAACUCC CU UCAAGGACAUCGUCCGGG AAAUGUUG | 9045 |
| 5162 | CAUUUGCA G AUGUUUUA | 3753 | UAAAACAU GGAGGAAACUCC CU UCAAGGACAUCGUCCGGG UGCAAAUG | 9056 |
| 5165 | UUGCAGAU G UUUUAGAA | 2711 | UUCUAAAA GGAGGAAACUCC CU UCAAGGACAUCGUCCGGG AUCUGCAA | 9047 |
| 5171 | AUGUUUUA G AAGGAAAA | 3754 | UUUUCCUU GGAGGAAACUCC CU UCAAGGACAUCGUCCGGG UAAAACAU | 9048 |
| 5174 | UUUUAGAA G GAAAAAAG | 3755 | CUUUUUUC GGAGGAAACUCC CU UCAAGGACAUCGUCCGGG UUCUAAAA | 9049 |
| 5175 | UUUAGAAC G AAAAAAGU | 3756 | ACUUUUUU GGAGGAAACUCC CU UCAAGGACAUCGUCCGCG CUUCUAAA | 9050 |
| 5182 | GGAAAAAA G UUCCUUCC | 2712 | GGAAGGAA GGAGGAAACUCC CU UCAAGGACAUCGUCCGGG UUUUUUCC | 9051 |
| 5212 | CUACAAUU G GAAGAUUG | 3757 | CAAUCUUC GGAGGAAACUCC CU UCAAGGACAUCGUCCGGG AAUUGUAC | 9052 |
| 5213 | UACAAUUG G AAGAUUCG | 3758 | CCAAUCUU GGAGGAAACUCC CU UCAAGGACAUCGUCCGGG CAAUUGUA | 9053 |
| 5216 | AAUUCGAA G AUUGGAAG | 3759 | CUUCCAAU GGAGGAAACUCC CU UCAAGGACAUCGUCCGGG UUCCAAUU | 9054 |
| 5220 | GGAAGAUU G GAAGAUUC | 3760 | GAAUCUUC GGAGGAAACUCC CU UCAAGGACAUCGUCCGGG AAUCUUCC | 9055 |
| 5221 | GAAGAUUG G AAGAUUCA | 3761 | UGAAUCUU GGAGGAAACUCC CU UCAAGGACAUCCUCCGGG UUCCAAUC | 9056 |
| 5224 | GAUUGGAA G AUUCAGCU | 3762 | AGCUGAAU GGAGGAAACUCC CU UCAAGGACAUCGUCCGGG UGAAUCUU | 9057 |
| 5230 | AACAUUCA G CUAGUUAG | 2713 | CUAACUAG GGAGGAAACUCC CU UCAAGGACAUCGUCCGGG UGAAUCUU | 9058 |
| 5234 | UUCAGCUA G UUAGGAGC | 2714 | GCUCCUAA GGAGGAAACUCC CU UCAAGGACAUCGUCCGGG UAGCUGAA | 9059 |
| 5038 | GCUAGUUA G GAGCCCAU | 3763 | AUGGGCUC GGAGGAAACUCC CU UCAAGGACAUCGUCCGGG UAACUAGC | 9060 |
| 5239 | CUAGUUAG G AGCCCAUU | 3764 | AAUGGGCU GGAGGAAACUCC CU UCAAGGACAUCGUCCGGG CUAACUAG | 9061 |
| 5214 | AGUUAGGA G CCCAUUUU | 2715 | AAAAUGGG GGAGGAAACUCC CU UCAAGGACAUCGUCCGGG UCCUAACU | 9062 |
| 5260 | CCUAAUCU G UGUGUGCC | 2716 | GGCACACA GGAGGAAACUCC CU UCAAGGACAUCGUCCGGG AGAUUAGG | 9063 |
| 5262 | UAAUCUGU G UGUGCCCU | 2717 | AGGGCACA GGAGGAAACUCC CU UCAAGGACAUCGUCCGGG ACAGAUUA | 9064 |
| 5264 | AUCUGUGU G UGCCCUGU | 2718 | ACAGGGCA GGAGGAAACUCC CU UCAAGGACAUCGUCCGGG ACACAGAU | 9065 |
| 5266 | CUGUGUGU G CCCUGUAA | 2719 | UUACAGGG GGAGGAAACUCC CU UCAAGGACAUCGUCCGGG ACACACAG | 9066 |
| 5271 | UGUGCCCU G UAACCUGA | 2720 | UCAGGUUA GGAGGAAACUCC CU UCAAGGACAUCGUCCGGG AGGGCACA | 9067 |
| 5278 | UGUAACCU G ACUGGUUA | 3765 | UAACCAGU GGAGGAAACUCC CU UCAAGGACAUCGUCCGGG AGGUUACA | 9068 |
| 5282 | ACCUGACU G GUUAACAG | 3766 | CUGUUAAC GGAGGAAACUCC CU UCAAGGACAUCGUCCGGG AGUCAGGU | 9069 |
| 5283 | CCUGACUG G UUAACAGC | 2721 | GCUGUUAA GGAGGAAACUCC CU UCAAGGACAUCGUCCGGG CAGUCAGG | 9070 |
| 5290 | GGUUAACA G CAGUCCUU | 2722 | AAGGACUG GGAGGAAACUCC CU UCAAGGACAUCGUCCGGG UGUUAACC | 9071 |
| 5293 | UAACAGCA G UCCUUUGU | 2723 | ACAAAGGA GGAGGAAACUCC CU UCAAGGACAUCGUCCGGG UGCUGUUA | 9072 |
| 5300 | AGUCCUUU G UAAACAGU | 2724 | ACUGUUUA GGAGGAAACUCC CU UCAAGGACAUCGUCCGGG AAAGGACU | 9073 |
| 5307 | UGUAAACA G UGUUUUAA | 2725 | UUAAAACA GGAGGAAACUCC CU UCAAGGACAUCGUCCGGG UGUUUACA | 9074 |
| 5309 | UAAACAGU G UUUUAAAC | 2726 | GUUUAAAA GGAGGAAACUCC CU UCAAGGACAUCGUCCGGG ACUGUUUA | 9075 |
| 5325 | CUCUCCUA G UCAAUAUC | 2727 | GAUAUUGA GGAGGAAACUCC CU UCAAGGACAUCGUCCGGG UAGGAGAG | 9076 |
| 5354 | UUUAUCAA G GAAGAAAU | 3767 | AUUUCUUC GGAGGAAACUCC CU UCAAGGACAUCGUCCGGG UUGAUAAA | 9077 |
| 5355 | UUAUCAAG G AAGAAAUG | 3768 | CAUUUCUU GGAGGAAACUCC CU UCAAGGACAUCGUCCGGG CUUGAUAA | 9078 |
| 5358 | UCAAGGAA G AAAUGGUU | 3769 | AACCAUUU GGAGGAAACUCC CU UCAAGGACAUCGUCCGGG UUCCUUGA | 9079 |
| 5363 | GAAGAAAU G GUUCAGAA | 3770 | UUCUGAAC GGAGGAAACUCC CU UCAAGGACAUCGUCCGGG AUUUCUUC | 9080 |
| 5364 | AAGAAAUG G UUCAGAAA | 2728 | UUUCUGAA GGAGGAAACUCC CU UCAAGGACAUCGUCCGGG CAUUUCUU | 9081 |
| 5369 | AUGGUUCA G AAAAUAUU | 3771 | AAUAUUUU GGAGGAAACUCC CU UCAAGGACAUCGUCCGGG UGAACCAU | 9082 |
| 5382 | UAUUUUCA G CCUACAGU | 2729 | ACUGUAGG GGAGGAAACUCC CU UCAAGGACAUCGUCCGGG UGAAAAUA | 9083 |
| 5389 | AGCCUACA G UUAUGUUC | 2730 | GAACAUAA GGAGGAAACUCC CU UCAAGGACAUCGUCCGGG UGUAGGCU | 9084 |
| 5394 | ACAGUUAU G UUCAGUCA | 2731 | UGACUGAA GGAGGAAACUCC CU UCAAGGACAUCGUCCGGG AUAACUGU | 9085 |
| 5399 | UAUGUUCA G UCACACAC | 2732 | GUGUGUGA GGAGGAAACUCC CU UCAAGGACAUCGUCCGGG UGAACAUA | 9086 |
| 5419 | UACAAAAU G UUCCUUUU | 2733 | AAAAGGAA GGAGGAAACUCC CU UCAAGGACAUCGUCCGGG AUUUUGUA | 9087 |
| 5428 | UUCCUUUU G CUUUUAAA | 2734 | UUUAAAAG GGAGGAAACUCC CU UCAAGGACAUCGUCCGGG AAAAGGAA | 9088 |
| 5437 | CUUUUAAA G UAAUUUUU | 2735 | AAAAAUUA GGAGGAAACUCC CU UCAAGGACAUCGUCCGGG UUUAAAAG | 9089 |
| 5446 | UAAUUUUU G ACUCCCAG | 3772 | CUGGGAGU GGAGGAAACUCC CU UCAAGGACAUCGUCCGGG AAAAAUUA | 9090 |
| 5454 | GACUCCCA G AUCAGUCA | 3773 | UGACUGAU GGAGGAAACUCC CU UCAAGGACAUCGUCCGGG UGGGAGUC | 9091 |
| 5459 | CCAGAUCA G UCAGAGCC | 2736 | GGCUCUGA GGAGGAAACUCC CU UCAAGGACAUCGUCCGGG UGAUCUGG | 9092 |
| 5463 | AUCAGUCA G AGCCCCUA | 3774 | UAGGGGCU GGAGGAAACUCC CU UCAAGGACAUCGUCCGGG UGACUGAU | 9093 |
| 5465 | CAGUCAGA G CCCCUACA | 2737 | UGUAGGGG GGAGGAAACUCC CU UCAAGGACAUCGUCCGGG UCUGACUG | 9094 |
| 5474 | CCCCUACA G CAUUGUUA | 2738 | UAACAAUG GGAGGAAACUCC CU UCAAGGACAUCGUCCGGG UGUAGGGG | 9095 |
| 5479 | ACAGCAUU G UUAAGAAA | 2739 | UUUCUUAA GGAGGAAACUCC CU UCAAGGACAUCGUCCGGG AAUGCUGU | 9096 |
| 5489 | AUUGUUAA G AAAGUAUU | 3775 | AAUACUUU GGAGGAAACUCC CU UCAAGGACAUCGUCCGGG UUAACAAU | 9097 |
| 5488 | UUAAGAAA G UAUUUGAU | 2740 | AUCAAAUA GGAGGAAACUCC CU UCAAGGACAUCGUCCGGG UUUCUUAA | 9098 |
| 5494 | AAGUAUUU G AUUUUUGU | 3776 | ACAAAAAU GGAGGAAACUCC CU UCAAGGACAUCGUCCGGG AAAUACUU | 9099 |
| 5501 | UGAUUUUU G UCUCAAUG | 2741 | CAUUGAGA GGAGGAAACUCC CU UCAAGGACAUCGUCCGGG AAAAAUCA | 9100 |
| 5509 | GUCUCAAU G AAAAUAAA | 3777 | UUUAUUUU GGAGGAAACUCC CU UCAAGGACAUCGUCCGGG AUUGAGAC | 9101 |

Input Sequence = NM_005228.1. Cut Site=G/.
Arm Length = 8. Core Sequence = GGAGGAAACUCC CU UCAAGGACAUCGUCCGGG
NM_005228.1 (Homo sapiens epidermal growth factor receptor (avian erythroblastic leukemia viral (v-erb-b) oncogene homolog) (EGFR), mRNA)

TABLE VIII
Human EGFR Receptor Nucleic Acid and Target molecules

| RPI# | Alias | Pos | SEQ ID | Target | Enzymatic Nucleic Acid/Antisense | SEQ ID |
|---|---|---|---|---|---|---|
| 5974 | egfr-146 Rz-7 allyl | 146 | 9102 | GUCCAGUAUUGAUCG | cₛgₛaₛuₛcaacUGAuGaggccgaaaggccGaaAcuggacB | 9353 |
| 5975 | egfr-549 Rz-7 allyl | 549 | 9103 | UAGCAGUCUUAUCUA | uₛaₛgₛaₛuaacUGAuGaggccgaaaggccGaaAcugcuaB | 9354 |
| 5976 | egfr-690 Rz-7 allyl | 690 | 9104 | ACAUAGUCAGCAGUG | cₛaₛcₛuₛgcucUGAuGaggccgaaaggccGaaAcuauguB | 9355 |
| 5977 | egfr-1165 Rz-6 allyl | 1165 | 9105 | CAAGUGUAAGAAGUG | aₛcₛuₛuₛcucUGAuGaggccgaaaggccGaaAcacuuB | 9356 |
| 5978 | egfr-1210 Rz-6 allyl | 1210 | 9106 | AAUAGGUAUUGGUGA | cₛaₛcₛcₛaacUGAuGaggccgaaaggccGaaAccuauB | 9357 |
| 5979 | egfr-2015 Rz-6 allyl | 2015 | 9107 | UGGAAGUACGCAGAC | uₛcₛuₛgₛcgcUGAuGaggccgaaaggccGaaAcuuccB | 9358 |
| 5980 | egfr-3126 Rz-6 allyl | 3126 | 9108 | ACCUUGUCAUUCAGG | cₛuₛgₛaₛaucUGAuGaggccgaaaggccGaaAcaaggB | 9359 |
| 5981 | egfr-4094 Rz-7 allyl | 4094 | 9109 | AAAGAGUAUAUUUGA | uₛcₛaₛaₛauacUGAuGaggccgaaaggccGaaAccucuuB | 9360 |
| 5982 | egfr-4165 Rz-7 allyl | 4165 | 9110 | UCAUUGUCGCUAUUG | cₛaₛaₛuₛagccUGAuGaggccgaaaggccGaaAcaaugaB | 9361 |
| 5983 | egfr-4280 Rz-6 allyl | 4280 | 9111 | CACAAGUCUUCCAGA | cₛuₛgₛgₛaacUGAuGaggccgaaaggccGaaAcuuguB | 9362 |
| 5984 | egfr-4394 Rz-6 allyl | 4394 | 9112 | AUCAAGUCAUGGCAG | uₛgₛcₛcₛaucUGAuGaggccgaaaggccGaaAcuugaB | 9363 |
| 5985 | egfr-4404 Rz-7 allyl | 4404 | 9113 | GGCAGGUACAGUAGG | cₛcₛuₛaₛcugcUGAuGaggccgaaaggccGaaAccugccB | 9364 |
| 5986 | egfr-4409 Rz-7 allyl | 4409 | 9114 | GUACAGUAGGAUAAG | cₛuₛuₛaₛucccUGAuGaggccgaaaggccGaaAcuguacB | 9365 |
| 5987 | egfr-4484 Rz-7 allyl | 4484 | 9115 | CUUUUGUAAAAAUGU | aₛcₛaₛuₛuuucUGAuGaggccgaaaggccGaaAcaaaagB | 9366 |
| 5988 | egfr-4501 Rz-6 allyl | 4501 | 9116 | CCACGGUACUUACUC | aₛgₛuₛaₛagcUGAuGaggccgaaaggccGaaAccgugB | 9367 |
| 5989 | egfr-4793 Rz-6 allyl | 4793 | 9117 | AUCAGGUCCUUUGGG | cₛcₛaₛaₛagcUGAuGaggccgaaaggccGaaAccugaB | 9368 |
| 5990 | egfr-4944 Rz-7 allyl | 4944 | 9118 | AAAGAGUAUAUHUUC | gₛaₛaₛcₛauacUGAuGaggccgaaaggccGaaAcucuuuB | 9369 |
| 5991 | egfr-5008 Rz-6 allyl | 5008 | 9119 | AAAGUGUCUCUGCCU | gₛgₛcₛaₛgacUGAuGaggccgaaaggccGaaAcacuuB | 9370 |
| 5992 | egfr-5110 Rz-7 allyl | 5110 | 9120 | UUCAGGUAGUAAAUA | uₛaₛuₛuₛuaccUGAuGaggccgaaaggccGaaAccugaaB | 9371 |
| 5993 | egfr-5401 Rz-7 allyl | 5401 | 9121 | GUUCAGUCACACACA | uₛgₛuₛgₛugucUGAuGaggccgaaaggccGaaAcugaacB | 9372 |
| 7540 | egfr-2015 Rz-6 allyl ALL P = O | 2015 | 9107 | UGGAAGUACGCAGAC | ucugcgcUGauGaggccgaaaggccGaaAcuuccB | 9373 |
| 11698 | GBC3.3 B 7/9/7 B | ctrl | | | BnnnnnnnNₛNₛNₛNₛNₛNₛNₛNₛnnnnnnnB | 9374 |
| 15340 | Sch09:557L23 poly-gln GB3.3 | ctrl | 9122 | AGCAGCAGCAGCAGCAGCAGCAG | BcugcugcTₛGₛCₛTₛGₛCₛTₛGₛCₛugcugcuB | 9375 |
| 19293 | erbB2-972 Zin.Rz-6 NH2 - | ctrl | | | gₛcₛaₛgₛuuggccgaaaggCgagugaGGuCuagcucaB | 9376 |
| 21083 | erbB2-972 Zin.Rz-6 trueSAC2 | ctrl | | | gₛgₛaₛcₛguugCacaugguacacguaCgacgaGGggB | 9377 |
| 21194 | EGFR-189 Amb.Rz-7 | 189 | 9123 | CAGCGAUGCGACCCU | aₛgₛgₛgₛucgggaLucCCUUCaaggaLucCGGGaucgcugB | 9378 |
| 21195 | EGFR-2825 Amb.Rz-6 | 2825 | 9124 | UCAAGUGGAUGGC | gₛcₛcₛaₛucggaLucCCUUCaaggaLucCGGGacuugaB | 9379 |
| 21196 | EGFR-2868 Amb.Rz-6 | 2868 | 9125 | CCACCAGAGUGAU | aₛuₛcₛaₛcuggaLucCCUUCaaggaLucCGGGugguggB | 9380 |
| 21197 | EGFR-2870 Amb.Rz-6 | 2870 | 9126 | ACCAGAGUGAUGU | aₛcₛaₛuₛcaggaLucCCUUCaaggaLucCGGGucugguB | 9381 |
| 21198 | EGFR-3021 Amb.Rz-6 | 3021 | 9127 | CUACAUGAUCAUG | cₛaₛuₛgₛauggaLucCCUUCaaggaLucCGGGauguagB | 9382 |
| 21199 | EGFR-3021 Amb.Rz-7 | 3021 | 9128 | UCUACAUGAUCAUGG | cₛcₛaₛuₛgauggaLucCCUUCaaggaLucCGGGauguagaB | 9383 |
| 21200 | EGFR-3250 Amb.Rz-6 | 3250 | 9129 | CAGCAGGGCUUCU | aₛgₛaₛaₛgcggaLucCCUUCaaggaLucCGGGcugcugB | 9384 |
| 21201 | EGFR-3251 Amb.Rz-6 | 3251 | 9130 | AGCAGGGCUUCUU | aₛaₛgₛaₛagggaLucCCUUCaaggaLucCGGGccugcuB | 9385 |
| 21202 | EGFR-62 Amb.Rz-7 | 62 | 9131 | GACGACAGGCCACCU | aₛgₛgₛuₛggcggaLucCCUUCaaggaLucCGGGugucgucB | 9386 |
| 21203 | EGFR-261 Amb.Rz-7 | 261 | 9132 | GGGCUCUGGAGGAAA | uₛuₛuₛcₛcucggaLucCCUUCaaggaLuc CGGGagagcccB | 9387 |
| 21204 | EGFR-582 Amb.Rz-7 | 582 | 9133 | CCGGACUGAAGGAGC | gₛcₛuₛcₛcuuggaLucCCUUCaaggaLucCGGGaguccggB | 9388 |
| 21205 | EGFR-585 Amb.Rz-7 | 585 | 9134 | GACUGAAGGAGCUGC | gₛcₛaₛgₛcucggaLucCCUUCaaggaLucCGGGuucagucB | 9389 |
| 21206 | EGFR-586 Amb.Rz-7 | 586 | 9135 | ACUGAAGGAGCUGCC | gₛgₛcₛaₛgcuggaLucCCUUCaaggaLucCGGGcuucaguB | 9390 |
| 21207 | EGFR-793 Amb.Rz-7 | 793 | 9136 | GGGUGCAGGAGAGGA | uₛcₛcₛuₛcucggaLucCCUUCaaggaLucCGGGugcacccB | 9391 |
| 21208 | EGFR-796 Amb.Rz-7 | 796 | 9137 | UGCAGGAGAGGAGAA | uₛuₛcₛuₛccuggaLucCCUUCaaggaLucCGGGuccugcaB | 9392 |
| 21209 | EGFR-801 Amb.Rz-7 | 801 | 9138 | GAGAGGAGAACUGCC | gₛgₛcₛaₛguuggaLucCCUUCaaggaLucCGGGuccucucB | 9393 |
| 21210 | EGFR-1141 Amb.Rz-7 | 1141 | 9139 | UGAGAUGGAGGAAGA | uₛcₛuₛuₛccuggaLucCCUUCaaggaLucCGGGcaucucaB | 9394 |
| 21211 | EGFR-1504 Amb.Rz-7 | 1504 | 9140 | UCUUGCAGUCGUCAG | cₛuₛgₛaₛcgaggaLucCCUUCaaggaLucCGGGugcaagaB | 9395 |
| 21212 | EGFR-1903 Amb.Rz-7 | 1903 | 9141 | CUGCACAGGACGGGG | cₛcₛcₛcₛgucggaLucCCUUCaaggaLucCGGGugugcagB | 9396 |
| 21213 | EGFR-1915 Amb.Rz-7 | 1915 | 9142 | GGGACCAGACAACUG | cₛaₛgₛuₛuguggaLucCCUUCaaggaLucCGGGuggucccB | 9397 |
| 21214 | EGFR-2241 Amb.Rz-7 | 2241 | 9143 | UGCAGGAGAGGGAGC | gₛcₛuₛcₛccuggaLucCCUUCaaggaLucCGGGuccugcaB | 9398 |
| 21215 | EGFR-2673 Amb.Rz-7 | 2673 | 9144 | ACUUGGAGGACCGUC | gₛaₛcₛgₛgucggaLucCCUUCaaggaLucCGGGuccaaguB | 9399 |
| 21216 | EGFR-2907 Amb.Rz-7 | 2907 | 9145 | GGGAGUUGAUGACCU | aₛgₛgₛuₛcauggaLucCCUUCaaggaLucCGGGaacucccB | 9400 |
| 21217 | EGFR-3433 Amb.Rz-7 | 3433 | 9146 | CAGCAUAGACGACAC | gₛuₛgₛuₛcguggaLucCCUUCaaggaLucCGGGuaugcugB | 9401 |
| 21218 | EGFR-3784 Amb.Rz-7 | 3784 | 9147 | CCUAAGGGUCGCGCC | gₛgₛcₛgₛcgaggaLucCCUUCaaggaLucCGGGccuuaggB | 9402 |
| 21219 | EGFR-3818 Amb.Rz-7 | 3818 | 9148 | GGAGCAUGACCACGG | cₛcₛgₛuₛgguggaLucCCUUCaaggaLucCGGGaugcuccB | 9403 |
| 21220 | EGFR-ctrl Amb.Rz-7 (SAC) | ctrl | 9149 | CAUUGUAGGCCACGC | gₛcₛgₛuₛggcggaLucCaaCUUCggaLucCGGGuacaaugB | 9404 |
| 21221 | EGFR-ctrl Amb.Rz-7 (SAC) | ctrl | 9150 | CGCAACUGGCUGUAC | gₛuₛaₛcₛagcggaLucCaaCUUCggaLucCGGGaguugcgB | 9405 |
| 21255 | EGFR-3017 Rz-6 allyl | 3017 | 9151 | AUGUCUACAUGAU | aₛuₛcₛaₛugcUGAuGaggccguuaggccGaaAgacaucB | 9406 |
| 21256 | EGFR-3017 Rz-7 allyl | 3017 | 9152 | GAUGUCUACAUGAUC | gₛaₛuₛcₛaugcUGAuGaggccguuaggccGaaAgacaucB | 9407 |
| 21257 | EGFR-3024 Rz-6 allyl | 3024 | 9153 | CAUGAUCAUGGUC | gₛaₛcₛcₛaucUGAuGaggccguuaggccGaaAucaugB | 9408 |
| 21258 | EGFR-3024 Rz-7 allyl | 3024 | 9154 | ACAUGAUCAUGGUCA | uₛgₛaₛcₛcaucUGAuGaggccguuaggccGaaAucauguB | 9409 |
| 21259 | EGFR-73 Rz-7 allyl | 73 | 9155 | ACCUCGUCGGCGUCC | gₛgₛaₛcₛgcccUGAuGaggccguuaggccGaaAcgagguB | 9410 |
| 21260 | EGFR-259 Rz-6 allyl | 259 | 9156 | CGGGCUCUGGAGG | cₛcₛuₛcₛcacUGAuGaggccguuaggccGaaAgcccgB | 9411 |
| 21261 | EGFR-448 Rz-7 allyl | 448 | 9157 | GGCUGGUUAUGUCCU | aₛgₛgₛaₛcaucUGAuGaggccguuaggccGaaAccagccB | 9412 |
| 21262 | EGFR-449 Rz-7 allyl | 449 | 9158 | GCUGGUUAUGUCCUC | gₛaₛgₛgₛacacUGAuGaggccguuaggccGaaAaccagcB | 9413 |
| 21263 | EGFR-456 Rz-7 allyl | 456 | 9159 | AUGUCCUCAUUGCCC | gₛgₛgₛcₛaaucUGAuGaggccguuaggccGaaAggacauB | 9414 |
| 21264 | EGFR-987 Rz-6 allyl | 987 | 9160 | CCCACUCAUGCUC | gₛaₛgₛcₛaucUGAuGaggccguuaggccGaaAgugggB | 9415 |
| 21265 | EGFR-993 Rz-7 allyl | 993 | 9161 | UCAUGCUCUACAACC | gₛgₛuₛuₛguacUGAuGaggccguuaggccGaaAgcaugaB | 9416 |
| 21266 | EGFR-1198 Rz-6 allyl | 1198 | 9162 | GUGUGUAACGGAA | uₛuₛcₛcₛgucUGAuGaggccguuaggccGaaAcacacB | 9417 |
| 21267 | EGFR-1206 Rz-7 allyl | 1206 | 9163 | ACGGAAUAGGUAUUG | cₛaₛaₛuₛacccUGAuGaggccguuaggccGaaAuuccguB | 9418 |
| 21268 | EGFR-1328 Rz-6 allyl | 1328 | 9164 | ACUCCUUCACACA | uₛgₛuₛgₛugcUGAuGaggccguuaggccGaaAggaguB | 9419 |
| 21269 | EGFR-1336 Rz-7 allyl | 1336 | 9165 | CACACAUACUCCUCC | gₛgₛaₛgₛgagcUGAuGaggccguuaggccGaaAugugugB | 9420 |
| 21270 | EGFR-1339 Rz-7 allyl | 1339 | 9166 | ACAUACUCCUCCUCU | aₛgₛaₛgₛgagcUGAuGaggccguuaggccGaaAguauguB | 9421 |
| 21271 | EGFR-1455 Rz-7 allyl | 1455 | 9167 | AGAACCUAGAAAUCA | uₛgₛaₛuₛuuccUGAuGaggccguuaggccGaaAgguucuB | 9422 |
| 21272 | EGFR-1500 Rz-7 allyl | 1500 | 9168 | UUUCUCUUGCAGUCG | cₛgₛaₛcₛugccUGAuGaggccguuaggccGaaAgagaaaB | 9423 |
| 21273 | EGFR-1506 Rz-6 allyl | 1506 | 9169 | UGCAGUCGUCAGC | gₛcₛuₛgₛaccUGAuGaggccguuaggccGaaAcugcaB | 9424 |
| 21274 | EGFR-1629 Rz-6 allyl | 1629 | 9170 | ACUGUUUGGGACC | gₛgₛuₛcₛcccUGAuGaggccguuaggccGaaAacaguB | 9425 |
| 21275 | EGFR-1833 Rz-7 allyl | 1833 | 9171 | GGGAGUUUGUGGAG | uₛcₛuₛcₛcaccUGAuGaggccguuaggccGaaAacucccB | 9426 |
| 21276 | EGFR-1944 Rz-7 allyl | 1944 | 9172 | ACUACAUUGACGGCC | gₛgₛcₛcₛguccUGAuGaggccguuaggccGaaAuguaguB | 9427 |
| 21277 | EGFR-2015 Rz-7 allyl | 2015 | 9107 | UGGAAGUACGCAGAC | gₛuₛcₛuₛgcgcUGAuGaggccguuaggccGaaAcuuccaB | 9428 |
| 21278 | EGFR-2205 Rz-7 allyl | 2205 | 9173 | GCCACAUCGUUCGGA | uₛcₛcₛgₛaaccUGAuGaggccguuaggccGaaAuguggcB | 9429 |
| 21279 | EGFR-2263 Rz-7 allyl | 2263 | 9174 | GCCUCUUACACCCAG | cₛuₛgₛgₛgugcUGAuGaggccguuaggccGaaAagaggcB | 9430 |
| 21280 | EGFR-2306 Rz-7 allyl | 2306 | 9175 | AGGAUCUUGAAGGAA | uₛuₛcₛcₛuuccUGAuGaggccguuaggccGaaAgauccuB | 9431 |
| 21281 | EGFR-2366 Rz-7 allyl | 2366 | 9176 | ACGGUGUAUAAGGGA | uₛcₛcₛcₛuuacUGAuGaggccguuaggccGaaAcaccguB | 9432 |
| 21282 | EGFR-2368 Rz-7 allyl | 2368 | 9177 | GGUGUAUAAGGGACU | aₛgₛuₛcₛccucUGAuGaggccguuaggccGaaAuacaccB | 9433 |
| 21283 | EGFR-2416 Rz-7 allyl | 2416 | 9178 | CGUCGCUAUCAAGGA | uₛcₛcₛuₛugacUGAuGaggccguuaggccGaaAgcgacgB | 9434 |
| 21284 | EGFR-2611 Rz-7 allyl | 2611 | 9179 | AGACAAUAUUGGCUC | gₛaₛgₛcₛcaacUGAuGaggccguuaggccGaaAuugucuB | 9435 |
| 21285 | EGFR-2624 Rz-7 allyl | 2624 | 9180 | UCCCAGUACCUGCUC | gₛaₛgₛcₛaggcUGAuGaggccguuaggccGaaAcugggaB | 9436 |
| 21286 | EGFR-2666 Rz-7 allyl | 2666 | 9181 | AUGAACUACUUGGAG | cₛuₛcₛcₛaagcUGAuGaggccguuaggccGaaAguucauB | 9437 |
| 21287 | EGFR-2669 Rz-6 allyl | 2669 | 9182 | ACUACUUGGAGGA | uₛcₛcₛuₛcccUGAuGaggccguuaggccGaaAguaguB | 9438 |
| 21288 | EGFR-2858 Rz-7 allyl | 2858 | 9183 | AGAAUCUAUACCCAC | gₛuₛgₛgₛguacUGAuGaggccguuaggccGaaAgauucuB | 9439 |
| 21289 | EGFR-2916 Rz-7 allyl | 2916 | 9184 | UGACCUUUGGAUCCA | uₛgₛgₛaₛucccUGAuGaggccguuaggccGaaAaggucaB | 9440 |
| 21290 | EGFR-3160 Rz-7 allyl | 3160 | 9185 | GCCAAGUCCUACAGA | uₛcₛuₛgₛuagcUGAuGaggccguuaggccGaaAcuuggcB | 9441 |
| 21291 | EGFR-3237 Rz-7 allyl | 3237 | 9186 | AGUACCUCAUCCCAC | gₛuₛgₛgₛgaucUGAuGaggccguuaggccGaaAgguacuB | 9442 |
| 21292 | EGFR-3444 Rz-7 allyl | 3444 | 9187 | ACACCUUCCUCCCAG | cₛuₛgₛgₛgagcUGAuGaggccguuaggccGaaAagguguB | 9443 |
| 21293 | EGFR-3473 Rz-6 allyl | 3473 | 9188 | ACCAGUCCGUUCC | gₛgₛaₛaₛcgcUGAuGaggccguuaggccGaaAcugguB | 9444 |
| 21294 | EGFR-3477 Rz-6 allyl | 3477 | 9189 | GUCCGUUCCCAAA | uₛuₛuₛgₛggcUGAuGaggccguuaggccGaaAcggacB | 9445 |
| 21295 | EGFR-3560 Rz-6 allyl | 3560 | 9190 | CACACUACCAGGA | uₛcₛcₛuₛggcUGAuGaggccguuaggccGaaAgugugB | 9446 |
| 21296 | EGFR-3639 Rz-7 allyl | 3639 | 9191 | GCACAUUCGACAGCC | gₛgₛcₛuₛguccUGAuGaggccguuaggccGaaAaugugcB | 9447 |
| 21297 | EGFR-3776 Rz-7 allyl | 3776 | 9192 | GCAGAAUACCUAAGG | cₛcₛuₛuₛaggcUGAuGaggccguuaggccGaaAuucugcB | 9448 |
| 21298 | EGFR-449 Rz-6 allyl | 449 | 9193 | CUGGUUAUGUCCU | aₛgₛgₛaₛcacUGAuGaggccguuaggccGaaAaccagB | 9449 |
| 21299 | EGFR-1072 Rz-7 allyl | 1072 | 9194 | GAAGUGUCCCCGUAA | uₛuₛaₛcₛgggcUGAuGaggccguuaggccGaaAcacuucB | 9450 |
| 21300 | EGFR-1252 Rz-7 allyl | 1252 | 9195 | UACGAAUAUUAAACA | uₛgₛuₛuₛuaacUGauGaggccguuaggccGaaAuucguaB | 9451 |
| 21301 | EGFR-1336 Rz-6 allyl | 1336 | 9196 | ACACAUACUCCUC | gₛaₛgₛgₛagcUGAuGaaccguuaggccGaaAuguguB | 9452 |
| 21302 | EGFR-1342 Rz- 6allyl | 1342 | 9197 | ACUCCUCCUCUGG | cₛcₛaₛgₛagcUGAuGaggccguuaggccGaaAggagB | 9453 |
| 21303 | EGFR-1498 Rz-7 allyl | 1498 | 9198 | GUUUUCUCUUGCAGU | aₛcₛuₛgₛcaacUGAuGaggccguuaggccGaaAgaaaacB | 9454 |
| 21304 | EGFR-2846 Rz-7 allyl | 2846 | 9199 | UCAAUUUUACACAGA | uₛcₛuₛgₛugucUGAuGaggccguuaggccGaaAaauugaB | 9455 |
| 21305 | EGFR-3465 Rz-7 allyl | 3465 | 9200 | AAUACAUAAACCAGU | aₛcₛuₛgₛguucUGAuGaggccguuaggccGaaAuguauuB | 9456 |
| 21306 | EGFR-3808 Rz-7 allyl | 3808 | 9201 | UGAAUUUAUUGGAGC | gₛcₛuₛcₛcaacUGAuGaggccguuaggccGaaAaauucaB | 9457 |
| 21307 | EGFR-370 CHz-6 allyl | 370 | 9202 | GUGGUCCUUGGGA | uₛcₛcₛcₛaacUGAuGaggccguuaggccGaaIaccacB | 9458 |
| 21308 | EGFR-900 CHz-6 allyl | 900 | 9203 | UGCUGCAGGCUGC | gₛcₛaₛgₛcccUGAuGaggccguuaggccGaaIcagcaB | 9459 |
| 21309 | EGFR-2524 CHz-6 allyl | 2524 | 9204 | CUGGGCAUCUGCC | gₛgₛcₛaₛgacUGAuGaggccguuaggccGaaIcccagB | 9460 |
| 21310 | EGFR-2866 CHz-6 allyl | 2866 | 9205 | ACCCACCAGAGUG | cₛaₛcₛuₛcucUGAuGaggccguuaggccGaaIuggguB | 9461 |
| 21311 | EGFR-2867 CHz-7 allyl | 2867 | 9206 | ACCCACCAGAGUGAU | aₛuₛcₛaₛcuccUGAuGaggccguuaggccGaaIguggguB | 9462 |
| 21312 | EGFR-3016 CHz-6 allyl | 3016 | 9207 | GAUGUCUACAUGA | uₛcₛaₛuₛgucUGAuGaggccguuaggccGaaIacaucB | 9463 |
| 21313 | EGFR-3019 CHz-6 allyl | 3019 | 9208 | GUCUACAUGAUCA | uₛgₛaₛuₛcacUGAuGaggccguuaggccGaaIuagacB | 9464 |
| 21314 | EGFR-3025 CHz-6 allyl | 3025 | 9209 | AUGAUCAUGGUCA | uₛgₛaₛcₛcacUGauGaggccguuaggccGaaIaucauB | 9465 |
| 21315 | EGFR-3112 CHz-6 allyl | 3112 | 9210 | GACCCCCAGCGCU | aₛgₛcₛgₛcucUGAuGaggccguuaggccGaaIgggucB | 9466 |
| 21316 | EGFR-143 CHz-7 allyl | 143 | 9211 | UCCGUCCAGUAUUGA | uₛcₛaₛaₛuaccUGauGaggccguuaggccGaaIgacggaB | 9467 |
| 21317 | EGFR-260 CHz-6 allyl | 260 | 9212 | GGGCUCUGGAGGA | uₛcₛcₛuₛcccUGAuGaggccguuaggccGaaIagcccB | 9468 |
| 21318 | EGFR-457 CHz-7 allyl | 457 | 9213 | UGUCCUCAUUGCCCU | aₛgₛgₛgₛcaacUGAuGaggccguuaggccGaaIaggcacaB | 9469 |
| 21319 | EGFR-469 CHz-7 allyl | 469 | 9214 | CCUCAACACAGUGGA | uₛcₛcₛaₛcugcUGAuGaggccguuaggccGaaIuugaggB | 9470 |
| 21320 | EGFR-471 CHz-7 allyl | 471 | 9215 | UCAACACAGUGGAGC | gₛcₛuₛcₛcaccUGAuGaggccguuaggccGaaIuguugaB | 9471 |
| 21321 | EGFR-505 CHz-7 allyl | 505 | 9216 | GCAGAUCAUCAGAGG | cₛcₛuₛcₛugacUGAuGaggccguuaggccGaaIaucugcB | 9472 |
| 21322 | EGFR-595 CHz-7 allyl | 595 | 9217 | GCUGCCCAUGAGAAA | uₛuₛuₛcₛucacUGAuGaggccguuaggccGaaIggcagcB | 9473 |
| 21323 | EGFR-640 CHz-7 allyl | 640 | 9218 | GUUCAGCAACAACCC | gₛgₛgₛuₛugucUGAuGaggccguuaggccGaaIcugaacB | 9474 |
| 21324 | EGFR-781 CHz-7 allyl | 781 | 9219 | UGGGAGCUGCUGGGG | cₛcₛcₛcₛagccUGauGaggccguuaggccGaaIcucccaB | 9475 |
| 21325 | EGFR-792 CHz-7 allyl | 792 | 9220 | GGGGUGCAGGAGAGG | cₛcₛuₛcₛucccUGAuGaggccguuaggccGaaIcaccccB | 9476 |
| 21326 | EGFR-994 CHz-7 allyl | 994 | 9221 | CAUGCUCUACAACCC | gₛgₛgₛuₛugucUGAuGaggccguuaggccGaaIagcaugB | 9477 |
| 21327 | EGFR-1125 CHz-7 allyl | 1125 | 9222 | GUGGGGCCGACAGCU | aₛgₛcₛuₛguccUGAuGaggccguuaggccGaaIccccacB | 9478 |
| 21328 | EGFR-1275 CHz-7 allyl | 1275 | 9223 | ACUGCACCUCCAUCA | uₛgₛaₛuₛggacUGAuGaggccguuaggccGaaIugcaguB | 9479 |
| 21329 | EGFR-1297 CHz-7 allyl | 1297 | 9224 | UCUCCACAUCCUGCC | gₛgₛcₛaₛggacUGAuGaggccguuaggccGaaIuggagaB | 9480 |
| 21330 | EGFR-1454 CHz-7 allyl | 1454 | 9225 | GAGAACCUAGAAAUC | gₛaₛuₛuₛucucUGAuGaggccguuaggccGaaIguucucB | 9481 |
| 21331 | EGFR-1503 CHz-7 allyl | 1503 | 9226 | CUCUUGCAGUCGUCA | uₛgₛaₛcₛgaccUGAuGaggccguuaggccGaaIcaagagB | 9482 |
| 21332 | EGFR-1514 CHz-7 allyl | 1514 | 9227 | GUCAGCCUGAACAUA | uₛaₛuₛgₛuuccUGAuGaggccguuaggccGaaIgcugacB | 9483 |
| 21333 | EGFR-1729 CHz-7 allyl | 1729 | 9228 | CGAGGGCUGCUGGGG | cₛcₛcₛcₛagccUGAuGaggccguuaggccGaaIcccucgB | 9484 |
| 21334 | EGFR-1824 CHz-7 allyl | 1824 | 9229 | GUGAGCCAAGGGAGU | aₛcₛuₛcₛccucUGAuGaggccguuaggccGaaIgcucacB | 9485 |
| 21335 | EGFR-1913 CHz-7 allyl | 1913 | 9230 | CGGGGACCAGACAAC | gₛuₛuₛgₛucuUGAuGaggccguuaggccGaaIuccccgB | 9486 |
| 21336 | EGFR-1914 CHz-6 allyl | 1914 | 9231 | GGGACCAGACAAC | gₛuₛuₛgₛuccUGAuGaggccguuaggccGaaIgucccB | 9487 |
| 21337 | EGFR-2030 CHz-7 allyl | 2030 | 9232 | GCCGGCCAUGUGUGC | gₛcₛaₛcₛacacUGAuGaggccguuaggccGaaIgccggcB | 9488 |
| 21338 | EGFR-2535 CHz-7 allyl | 2535 | 9233 | GCCUCACCUCCACCG | cₛgₛgₛuₛggacUGAaGaggccguuaggccGaaIugaggcB | 9489 |
| 21339 | EGFR-2541 CHz-7 allyl | 2541 | 9234 | CCUCCACCGUGCAAC | gₛuₛuₛgₛcaccUGAuGaggccguuaggccGaaIuggaggB | 9490 |
| 21340 | EGFR-2546 CHz-7 allyl | 2546 | 9235 | ACCGUGCAACUCAUC | gₛaₛuₛgₛagucUGAuGaggccguuaggccGaaIcacgguB | 9491 |
| 21341 | EGFR-2668 CHz-7 allyl | 2668 | 9236 | GAACUACUUGGAGGA | uₛcₛcₛuₛccacUGAuGaggccguuaggccGaaIuaguucB | 9492 |
| 21342 | EGFR-2677 CHz-7 allyl | 2677 | 9237 | GGAGGACCGUCGCUU | aₛaₛgₛcₛgaccUGAuGaggccguuaggccGaaIuccuccB | 9493 |
| 21343 | EGFR-2941 CHz-7 allyl | 2941 | 9238 | CGGAAUCCCUGCCAG | cₛuₛgₛgₛcagcUGAuGaggccguuaggccGaaIauuccgB | 9494 |
| 21344 | EGFR-3111 CHz-7 allyl | 3111 | 9239 | GAGACCCCCAGCGCU | aₛgₛcₛgₛcugcUGAuGaggccguuaggccGaaIggucucB | 9495 |
| 21345 | EGFR-3121 CHz-7 allyl | 3121 | 9240 | GCGCUACCUUGUCAU | aₛuₛgₛaₛcaacUGAuGaggccguuaggccGaaIuagcgcB | 9496 |
| 21346 | EGFR-3122 CHz-7 allyl | 3122 | 9241 | CGCUACCUUGUCAUU | aₛaₛuₛgₛacacUGAuGaggccguuaggccGaaIguagcgB | 9497 |
| 21347 | EGFR-3188 CHz-7 allyl | 3188 | 9242 | CGUGCCCUGAUGGAU | aₛuₛcₛcₛauccUGAuGaggccguuaggccGaaIggcacgB | 9498 |
| 21348 | EGFR-3238 CHz-7 allyl | 3238 | 9243 | GUACCUCAUCCCACA | uₛgₛuₛgₛggacUGAuGaggccguuaggccGaaIagguacB | 9499 |
| 21349 | EGFR-3286 CHz-7 allyl | 3286 | 9244 | GACUCCCCUCCUGAG | cₛuₛcₛaₛggacUGAuGaggccguuaggccGaaIggagucB | 9500 |
| 21350 | EGFR-3445 CHz-7 allyl | 3445 | 9245 | CACCUUCCUCCCAGU | aₛcₛuₛgₛggacUGAuGaggccguuaggccGaaIaaggugB | 9501 |
| 21351 | EGFR-3446 CHz-7 allyl | 3446 | 9246 | ACCUUCCUCCCAGUG | cₛaₛcₛuₛgggcUGAuGaggccguuaggccGaaIgaagguB | 9502 |
| 21352 | EGFR-3474 CHz-6 allyl | 3474 | 9247 | CCAGUCCGUUCCC | gₛgₛgₛaₛaccUGAuGaggccguuaggccGaaIacuggB | 9503 |
| 21353 | EGFR-3536 CHz-7 allyl | 3536 | 9248 | CUGAACCCCGCGCCC | gₛgₛgₛcₛgcgcUGAuGaggccguuaggccGaaIguucagB | 9504 |
| 21354 | EGFR-3554 CHz-7 allyl | 3554 | 9249 | AGAGACCCACACUAC | gₛuₛaₛgₛugucUGAuGaggccguuaggccGaaIgucucuB | 9505 |
| 21355 | EGFR-3555 CHz-7 allyl | 3555 | 9250 | GAGACCCACACUACC | gₛgₛuₛaₛgugcUGAuGaggccguuaggccGaaIggucucB | 9506 |
| 21356 | EGFR-3557 CHz-6 allyl | 3557 | 9251 | ACCCACACUACCA | uₛgₛgₛuₛagcUGauGaggccguuaggccGaaIuggguB | 9507 |
| 21357 | EGFR-3562 CHz-7 allyl | 3562 | 9252 | ACACUACCAGGACCC | gₛgₛgₛuₛccucUGAuGaggccguuaggccGaaIuaguguB | 9508 |
| 21358 | EGFR-3570 CHz-7 allyl | 3570 | 9253 | AGGACCCCCACAGCA | uₛgₛcₛuₛgugcUGAuGaggccguuaggccGaaIgguccuB | 9509 |
| 21359 | EGFR-189 Zin.Rz-7 NH2 | 189 | 9123 | CAGCGAUGCGACCCU | aₛgₛgₛgₛucggccgaaaggCgagugaGguCuaucgcugB | 9510 |
| 21360 | EGFR-2870 Zin.Rz-6 NH2 | 2870 | 9126 | ACCAGAGUGAUGU | aₛcₛaₛuₛcagccgaaaggCgagugaGguCuucugguB | 9511 |
| 21361 | EGFR-3251 Zin.Rz-6 NH2 | 3251 | 9130 | AGCAGGGCUUCUU | aₛaₛgₛaₛaggccgaaaggCgagugaGguCuccugcuB | 9512 |
| 21362 | EGFR-3251 Zin.Rz-7 NH2 | 3251 | 9254 | CAGCAGGGCUUCUUC | gₛaₛaₛgₛaaggccgaaaggCgagugaGguCuccugcugB | 9513 |
| 21363 | EGFR-24 Zin.Rz-6 NH2 | 24 | 9255 | UCCCGAGCUAGCC | gₛgₛcₛuₛaggccgaaaggCgagugaGguCuucgggaB | 9514 |
| 21364 | EGFR-98 Zin.Rz-6 NH2 | 98 | 9256 | CGCCUCGCCGCCA | uₛgₛgₛcₛgggccgaaaggCgagugaGguCugaggcgB | 9515 |
| 21365 | EGFR-98 Zin.Rz-7 NH2 | 98 | 9257 | CCGCCUCGCCGCCAA | uₛuₛgₛgₛcgggccgaaaggCgagugaGguCugaggcggB | 9516 |
| 21366 | EGFR-101 Zin.Rz-7 NH2 | 101 | 9258 | CCUCGCCGCCAACGC | gₛcₛgₛuₛugggccgaaaggCgagugaGguCuggcgaggB | 9517 |
| 21367 | EGFR-139 Zin.Rz-6 NH2 | 139 | 9259 | GACUCCGUCCAGU | aₛcₛuₛgₛgagccgaaaggCgagugaGguCuggagucB | 9518 |
| 21368 | EGFR-139 Zin.Rz-7 NH2 | 139 | 9260 | UGACUCCGUCCAGUA | uₛaₛcₛuₛggagccgaaaggCgagugaGguCuggagucaB | 9519 |
| 21369 | EGFR-472 Zin.Rz-7 NH2 | 472 | 9261 | CAACACAGUGGAGCG | cₛgₛcₛuₛccagccgaaaggCgagugaGguCuuguguugB | 9520 |
| 21370 | EGFR-588 Zin.Rz-6 NH2 | 588 | 9262 | GAAGGAGCUGCCC | gₛgₛgₛcₛaggccgaaaggCgagugaGguCuuccuucB | 9521 |
| 21371 | EGFR-588 Zin.Rz-7 NH2 | 588 | 9263 | UGAAGGAGCUGCCCA | uₛgₛgₛgₛcaggccgaaaggCgagugaGguCuuccuucaB | 9522 |
| 21372 | EGFR-714 Zin.Rz-7 NN2 | 714 | 9264 | GCAACAUGUCGAUGG | cₛcₛaₛuₛcgagccgaaaggCgagugaGguCuauguugcB | 9523 |
| 21373 | EGFR-782 Zin.Rz-7 NH2 | 782 | 9265 | GGGAGCUGCUGGGGU | aₛcₛcₛcₛcaggccgaaaggCgagugaGguCuagcucccB | 9524 |
| 21374 | EGFR-923 Zin.Rz-7 NH2 | 923 | 9266 | CGGGAGAGCGACUGC | gₛcₛaₛgₛucggccgaaaggCgagugaGguCuucucccgB | 9525 |
| 21375 | EGFR-990 Zin.Rz-7 NH2 | 990 | 9267 | CACUCAUGCUCUACA | uₛgₛuₛaₛgaggccgaaaggCgagugaGguCuaugagugB | 9526 |
| 21376 | EGFR-1008 Zin.Rz-7 NH2 | 1008 | 9268 | CCACCACGUACCAGA | uₛcₛuₛgₛguagccgaaaggCgagugaGguCugugguggB | 9527 |
| 21377 | EGFR-1501 Zin.Rz-7 NH2 | 1501 | 9269 | UUCUCUUGCAGUCGU | aₛcₛgₛaₛcuggccgaaaggCgagugaGguCuaagagaaB | 9528 |
| 21378 | EGFR-1504 Zin.Rz-7 NH2 | 1504 | 9140 | UCUUGCAGUCGUCAG | cₛuₛgₛaₛcgagccgaaaggCgagugaGguCuugcaagaB | 9529 |
| 21379 | EGFR-1507 Zin.Rz-6 NH2 | 1507 | 9270 | GCAGUCGUCAGCC | gₛgₛcₛuₛgagccgaaaggCgagugaGguCugacugcB | 9530 |
| 21380 | EGFR-1507 Zin.Rz-7 NH2 | 1507 | 9271 | UGCAGUCGUCAGCCU | aₛgₛgₛcₛugagccgaaaggCgagugaGguCugacugcaB | 9531 |
| 21381 | EGFR-1640 Zin.Rz-7 NH2 | 1640 | 9272 | ACCUCCGGUCAGAAA | uₛuₛuₛcₛugagccgaaaggCgagugaGguCucggagguB | 9532 |
| 21382 | EGFR-1817 Zin.Rz-6 NH2 | 1817 | 9273 | UGGAGGGUGAGCC | gₛgₛcₛuₛcagccgaaaggCgagugaGguCuccuccaB | 9533 |
| 21383 | EGFR-1929 Zin.Rz-7 NH2 | 1929 | 9274 | GUAUCCAGUGUGCCC | gₛgₛgₛcₛacagccgaaaggCgagugaGguCuuggauacB | 9534 |
| 21384 | EGFR-1931 Zin.Rz-7 NH2 | 1931 | 9275 | AUCCAGUGUGCCCAC | gₛuₛgₛgₛgcagccgaaaggCgagugaGguCuacuggauB | 9535 |
| 21385 | EGFR-2034 Zin.Rz-7 NH2 | 2034 | 9276 | GCCAUGUGUGCCACC | gₛgₛuₛgₛgcagccgaaaggCgagugaGguCuacauggcB | 9536 |
| 21386 | EGFR-2043 Zin.Rz-7 NH2 | 2043 | 9277 | GCCACCUGUGCCAUC | gₛaₛuₛgₛgcagccgaaaggCgagugaGguCuagguggcB | 9537 |
| 21387 | EGFR-2542 Zin.Rz-6 NH2 | 2542 | 9278 | UCCACCGUGCAAC | gₛuₛuₛgₛcagccgaaaggCgagugaGguCugguggaB | 9538 |
| 21388 | EGFR-2542 Zin.Rz-7 NH2 | 2542 | 9279 | CUCCACCGUGCAACU | aₛgₛuₛuₛgcagccgaaaggCgagugaGguCugguggagB | 9539 |
| 21389 | EGFR-2544 Zin.Rz-7 NH2 | 2544 | 9280 | CCACCGUGCAACUCA | uₛgₛaₛgₛuuggccgaaaggCgagugaGguCuacgguggB | 9540 |
| 21390 | EGFR-2622 Zin.Rz-7 NH2 | 2622 | 9281 | GCUCCCAGUACCUGC | gₛcₛaₛgₛguagccgaaaggCgagugaGguCuugggagcB | 9541 |
| 21391 | EGFR-2639 Zin.Rz-7 NH2 | 2639 | 9282 | AACUGGUGUGUGCAG | cₛuₛgₛcₛacagccgaaaggCgagugaGguCuaccaguuB | 9542 |
| 21392 | EGFR-2641 Zin.Rz-7 NH2 | 2641 | 9283 | CUGGUGUGUGCAGAU | aₛuₛcₛuₛgcagccgaaaggCgagugaGguCuacaccagB | 9543 |
| 21393 | EGFR-2678 Zin.Rz-7 NH2 | 2678 | 9284 | GAGGACCGUCGCUUG | cₛaₛaₛgₛcgagccgaaaggCgagugaGguCugguccucB | 9544 |
| 21394 | EGFR-2948 Zin.Rz-7 NH2 | 2948 | 9285 | CCUGCCAGCGAGAUC | gₛaₛuₛcₛucggccgaaaggCgagugaGguCuuggcaggB | 9545 |
| 21395 | EGFR-3033 Zin.Rz-7 NH2 | 3033 | 9286 | UGGUCAAGUGCUGGA | uₛcₛcₛaₛgcagccgaaaggCgagugaGguCuuugaccaB | 9546 |
| 21396 | EGFR-3056 Zin.Rz-7 NH2 | 3056 | 9287 | GCAGAUAGUCGCCCA | uₛgₛgₛgₛcgagccgaaaggCgagugaGguCuuaucugcB | 9547 |
| 21397 | EGFR-3066 Zin.Rz-7 NH2 | 3066 | 9288 | GCCCAAAGUUCCGUG | cₛaₛcₛgₛgaagccgaaaggCgagugaGguCuuuugggcB | 9548 |
| 21398 | EGFR-3124 Zin.Rz-7 NH2 | 3124 | 9289 | CUACCUUGUCAUUCA | uₛgₛaₛaₛugagccgaaaggCgagugaGguCuaagguagB | 9549 |
| 21399 | EGFR-3158 Zin.Rz-7 NH2 | 3158 | 9290 | UUGCCAAGUCCUACA | uₛgₛuₛaₛggagccgaaaggCgagugaGguCuuuggcaaB | 9550 |
| 21400 | EGFR-3182 Zin.Rz-7 NH2 | 3182 | 9291 | UUCUACCGUGCCCUG | cₛaₛgₛgₛgcagccgaaaggCgagugaGguCugguagaaB | 9551 |
| 21401 | EGFR-3214 Zin.Rz-7 NH2 | 3214 | 9292 | GGACGACGUGGUGGA | uₛcₛcₛaₛccagccgaaaggCgagugaGguCugucguccB | 9552 |
| 21402 | EGFR-3293 Zin.Rz-7 NH2 | 3293 | 9293 | CUCCUGAGCUCUCUG | cₛaₛgₛaₛgaggccgaaaggCgagugaGguCuucaggagB | 9553 |
| 21402 | EGFR-3302 Zin.Rz-7 NH2 | 3202 | 9294 | UCUCUGAGUGCAACC | gₛgₛuₛuₛgcagccgaaaggCgagugaGguCuucagagaB | 9554 |
| 21404 | EGFR-3410 Zin.Rz-7 NH2 | 3410 | 9295 | CCCACAGGCGCCUUG | cₛaₛaₛgₛgcggccgaaaggCgagugaGguCucugugggB | 9555 |
| 21405 | EGFR-3451 Zin.Rz-7 NH2 | 3451 | 9296 | CCUCCCAGUGCCUGA | uₛcₛaₛgₛgcagccgaaaggCgagugaGguCuugggaggB | 9556 |
| 21406 | EGFR-3471 Zin.Rz-7 NH2 | 3471 | 9297 | UAAACCAGUCCGUUC | gₛaₛaₛcₛggagccgaaaggCgagugaGguCuugguuuaB | 9557 |
| 21407 | EGFR-3475 Zin.Rz-6 NH2 | 3475 | 9298 | CAGUCCGUUCCCA | uₛgₛgₛgₛaagccgaaaggCgagugaGguCuggacugB | 9558 |
| 21408 | EGFR-3475 Zin.Rz-7 NH2 | 3475 | 9299 | CCAGUCCGUUCCCAA | uₛuₛgₛgₛgaagccgaaaggCgagugaGguCuggacuggB | 9559 |
| 21409 | EGFR-3623 Zin.Rz-7 NH2 | 3623 | 9300 | CCCACCUGUGUCAAC | gₛuₛuₛgₛacagccgaaaggCgagugaGguCuaggugggB | 9560 |
| 21410 | EGFR-3671 Zin.Rz-7 NH2 | 3671 | 9301 | AAAGGCAGCCACCAA | uₛuₛgₛgₛugggccgaaaggCgagugaGguCuugccuuuB | 9561 |
| 21411 | EGFR-3784 Zin.Rz-7 NH2 | 3784 | 9147 | CCUAAGGGUCGCGCC | gₛgₛcₛgₛcgagccgaaaggCgagugaGguCuccuuaggB | 9562 |
| 21412 | EGFR-188 AHz-7 NH2 | 188 | 9302 | GCAGCGAUGCGACCC | gₛgₛgₛuₛcgccUGAUGaggccguuaggccGaaUcgcugcB | 9563 |
| 21413 | EGFR-2525 AHz-6 NH2 | 2525 | 9303 | UGGGCAUCUGCCU | aₛgₛgₛcₛagcUGAUGaggccguuaggccGaaUgcccaB | 9564 |
| 21414 | EGFR-3018 AHz-6 NH2 | 3018 | 9304 | UGUCUACAUGAUC | gₛaₛuₛcₛaucUGAUGaggccguuaggccGaaUagacaB | 9565 |
| 21415 | EGFR-3018 AHz-7 NH2 | 3018 | 9305 | AUGUCUACAUGAUCA | uₛgₛaₛuₛcaucUGAUGaggccguuaggccGaaUagacauB | 9566 |
| 21416 | EGFR-3020 AHz-6 NH2 | 3020 | 9306 | UCUACAUGAUCAU | aₛuₛgₛaₛuccUGAUGaggccguuaggccGaaUguagaB | 9567 |
| 21417 | EGFR-3020 AHz-7 NH2 | 3020 | 9307 | GUCUACAUGAUCAUG | cₛaₛuₛgₛauccUGAUGaggccguuaggccGaaUguagacB | 9568 |
| 21418 | EGFR-3023 AHz-6 NH2 | 3023 | 9308 | ACAUGAUCAUGGU | aₛcₛcₛaₛugcUGAUGaggccguuaggccGaaUcauguB | 9569 |
| 21419 | EGFR-3023 AHz-7 NH2 | 3023 | 9309 | UACAUGAUCAUGGUC | gₛaₛcₛcₛaugcUGAUGaggccguuaggccGaaUcauguaB | 9570 |
| 21420 | EGFR-3026 AHz-6 NH2 | 3026 | 9310 | UGAUCAUGGUCAA | uₛuₛgₛaₛcccUGAUGaggccguuaggccGaaUgaucaB | 9571 |
| 21421 | EGFR-467 AHz-7 NH2 | 467 | 9311 | GCCCUCAACACAGUG | cₛaₛcₛuₛgugcUGAUGaggccguuaggccGaaUgagggcB | 9572 |
| 21422 | EGFR-641 AHz-7 NH2 | 641 | 9312 | UUCAGCAACAACCCU | aₛgₛgₛgₛuugcUGAUGaggccguuaggccGaaUgcugaaB | 9573 |
| 21423 | EGFR-999 AHz-7 NH2 | 999 | 9313 | UCUACAACCCCACCA | uₛgₛgₛuₛgggcUGAUGaggccguuaggccGaaUuguagaB | 9574 |
| 21424 | EGFR-1037 AHz-7 NH2 | 1037 | 9314 | GAGGGCAAAUACAGC | gₛcₛuₛgₛuaucUGAUGaggccguuaggccGaaUgcccucB | 9575 |
| 21425 | EGFR-1146 AHz-7 NH2 | 1146 | 9315 | UGGAGGAAGACGGCG | cₛgₛcₛcₛguccUGAUGaggccguuaggccGaaUccuccaB | 9576 |
| 21426 | EGFR-1335 AHz-7 NH2 | 1335 | 9316 | UCACACAUACUCCUC | gₛaₛgₛgₛagucUGAUGaggccguuaggccGaaUgugugaB | 9577 |
| 21427 | EGFR-1459 AHz-7 NH2 | 1459 | 9317 | CCUAGAAAUCAUACG | cₛgₛuₛaₛugacUGAUGaggccguuaggccGaaUucuaggB | 9578 |
| 21428 | EGFR-1993 AHz-7 NH2 | 1993 | 9318 | GGGAGAAAACAACAC | gₛuₛgₛuₛugucUGAUGaggccguuaggccGaaUucucccB | 9579 |
| 21429 | EGFR-2367 AHz-7 NH2 | 2367 | 9319 | CGGUGUAUAAGGGAC | gₛuₛcₛcₛcuucUGAUGaggccguuaggccGaaUacaccgB | 9580 |
| 21430 | EGFR-2540 AHz-7 NH2 | 2540 | 9320 | ACCUCCACCGUGCAA | uₛuₛgₛcₛacgcUGAUGaggccguuaggccGaaUggagguB | 9581 |
| 21431 | EGFR-2660 AHz-7 NH2 | 2660 | 9321 | AAGGGCAUGAACUAC | gₛuₛaₛgₛuuccUGAUGaggccguuaggccGaaUgcccuuB | 9582 |
| 21432 | EGFR-2781 AHz-7 NH2 | 2781 | 9322 | GUGCGGAAGAGAAAG | cₛuₛuₛuₛcuccUGAUGaggccguuaggccGaaUccgcacB | 9583 |
| 21433 | EGFR-3317 AHz-7 NH2 | 3317 | 9323 | AGCAACAAUUCCACC | gₛgₛuₛgₛgaacUGAUGaggccguuaggccGaaUguugcuB | 9584 |
| 21434 | EGFR-3318 AHz-7 NH2 | 3318 | 9324 | GCAACAAUUCCACCG | cₛgₛgₛuₛggacUGAUGaggccguuaggccGaaUuguugcB | 9585 |
| 21435 | EGFR-3467 AHz-7 NH2 | 3467 | 9325 | UACAUAAACCAGUCC | gₛgₛaₛcₛuggcUGAUGaggccguuaggccGaaUuauguaB | 9586 |
| 21436 | EGFR-3556 AHz-7 NH2 | 3556 | 9326 | AGACCCACACUACCA | uₛgₛgₛuₛagucUGAUGaggccguuaggccGaaUgggucuB | 9587 |
| 21437 | EGFR-3558 AHz-6 NH2 | 3558 | 9327 | CCCACACUACCAG | cₛuₛgₛgₛuacUGAUGaggccguuaggccGaaUgugggB | 9588 |
| 21438 | EGFR-3605 AHz-7 NH2 | 3605 | 9328 | UAUCUCAACACUGUC | gₛaₛcₛaₛgugcUGAUGaggccguuaggccGaaUgagauaB | 9589 |
| 21439 | EGFR-ctrl Rz-7 allyl | ctrl | 9329 | CGCACAUHCCAGGUU | aₛaₛcₛcₛuggcUAguGacgccguuaggcgGaaAugugcgB | 9590 |
| 21440 | EGFR-ctrl Rz-7 allyl | ctrl | 9330 | CGCAACUHGCUGUAC | gₛuₛaₛcₛagccUAGuGacgccguuaggcgGaaAguugcgB | 9591 |
| 21441 | EGFR-ctrl CHz-7 allyl | ctrl | 9331 | CUACCGCHUAACGUG | cₛaₛcₛgₛuuacUAGuGacgccguuaggcgGaaIcgguagB | 9592 |
| 21442 | EGFR-ctrl CHz-7 allyl | ctrl | 9332 | GACUUGCHGUAACCC | gₛgₛgₛuₛuaccUAGuGacgccguuaggcgGaaIcaagucB | 9593 |
| 21443 | EGFR-ctrl AHz-7 NH2 | ctrl | 9333 | CGCUUCAHCAGAUGC | gₛcₛaₛuₛcugcUAGUGacgccguuaggcgGaaUgaagcgB | 9594 |
| 21444 | EGFR-ctrl AHz-7 NH2 | ctrl | 9334 | CAUUGUAHGCCACGC | gₛcₛgₛuₛggccUAGUGacgccguuaggcgGaaUacaaugB | 9595 |
| 21445 | EGFR-ctrl Zin.Rz-7 NH2 (SAC) | ctrl | 9149 | CAUUGUAGGCCACGC | gₛcₛgₛuₛggcgccgaaaggCuCugGagugaguacaaugB | 9596 |
| 21446 | EGFR-ctrl Zin.Rz-7 NH2 (SAC) | ctrl | 9150 | CGCAACUGGCUGUAC | gₛuₛaₛcₛagcgccgaaaggCuCugGagugagaguugcgB | 9597 |
| 21542 | EGFR:788L20 GB3.3 | 788 | 9335 | CCCAAUGGGAGCUGCUGGGG | BccccagCₛAₛGₛCₛTₛCₛCₛCₛAₛuugggb | 9598 |
| 21543 | EGFR:821L23 GB3.3 | 821 | 9336 | GAGAACUGCCAGAAACUGACCAA | BuuggucaGₛTₛTₛTₛCₛTₛGₛGₛCₛaguucucB | 9599 |
| 21544 | EGFR:1129L20 GB3.3 | 1129 | 9337 | CCGAGCCUGUGGGGCCGACA | BugucggCₛCₛCₛCₛAₛCₛAₛGₛGₛcucggB | 9600 |
| 21545 | EGFR:1402L23 GB3.3 | 1402 | 9338 | AAAGGAAAUCACAGGGUUUUUGC | BgcaaaaaCₛCₛCₛTₛGₛTₛGₛAₛTₛuuccuuuB | 9601 |
| 21546 | EGFR:1780L20 GB3.3 | 1780 | 9339 | UUGCCGGAAUGUCAGCCGAG | BcucggcTₛGₛAₛCₛAₛTₛTₛCₛCₛggcaaB | 9602 |
| 21547 | EGFR:3174L23 GB3.3 | 3174 | 9340 | UGCCAAGUCCUACAGACUCCAAC | BguuggagTₛCₛTₛGₛTₛAₛGₛGₛAₛcuuggcaB | 9603 |
| 21548 | EGFR:3825L15 GB3.3 | 3825 | 9148 | GGAGCAUGACCACGG | BccgTₛGₛGₛTₛCₛAₛTₛGₛCₛuccB | 9604 |
| 21549 | EGFR:4157L23 GB3.3 | 4157 | 9341 | UUGAUUGGGGAUCUUGGAGUUUU | BaaaacucCₛAₛAₛGₛAₛTₛCₛCₛCₛcaaucaaB | 9605 |
| 21550 | EGFR:3830L23 GB3.3 | 3830 | 9342 | AUUGGAGCAUGACCACGGAGGAU | BauccuccGₛTₛGₛGₛTₛCₛAₛTₛGₛcuccaauB | 9606 |
| 24254 | EGFR-3017 Rz-7 allyl (SAC) | 3017 | 9343 | ACGCUAUAUUGUCGA | uₛcₛgₛaₛcaacUAGuGacgccguuaggcgGaaAuagcguB | 9607 |
| 24255 | EGFR-448 Rz-7 allyl (SAC) | 448 | 9344 | UCUGGUUUCGUAGGC | gₛcₛcₛuₛacgcUAGuGacgccguuaggcgGaaAaccagaB | 9608 |
| 24256 | EGFR-1455 Rz-7 allyl (SAC) | 1455 | 9345 | CCGAAUUAAACGAAA | uₛuₛuₛcₛguucUAGuGacgccguuaggcgGaaAauucggB | 9609 |
| 24257 | EGFR-1506 Rz-6 allyl (SAC) | 1506 | 9346 | GAUGCUCGCGCUA | uₛaₛgₛcₛgccUAGuGacgccguuaggcgGaaAgcaucB | 9610 |
| 24258 | EGFR-3473 Rz-6 allyl (SAC) | 3473 | 9347 | CCGUCUCUCAGCA | uₛgₛcₛuₛgacUAGuGacgccguuaggcgGaaAgacggB | 9611 |
| 24259 | EGFR-900 CHz-6 allyl (SAC) | 900 | 9348 | GCUGCCAGUCGUG | cₛaₛcₛgₛaccUAGuGacgccguuaggcgGaaIgcagcB | 9612 |
| 24260 | EGFR-1514 CHz-7 allyl (SAC) | 1514 | 9349 | CCAUGGCUUGAACAA | uₛuₛgₛuₛucacUAGuGacgccguuaggcgGaaIccauggB | 9613 |
| 24261 | EGFR-2535 CHz-7 allyl (SAC) | 2535 | 9350 | CGUCCGCCACACCUC | gₛaₛgₛgₛugucUAGuGacgccguuaggcgGaaIcggacgB | 9614 |
| 24262 | EGFR-3122 CHz-7 allyl (SAC) | 3122 | 9351 | CCUAUUCUCGCGUUA | uₛaₛaₛcₛgcgcUAGuGacgccguuaggcgGaaIaauaggB | 9615 |
| 24263 | EGFR-3188 CHz-7 allyl (SAC) | 3188 | 9352 | AGCCGACUUUCGUGG | cₛcₛaₛcₛgaacUAGuGacgccguuaggcgGaaIucggcuB | 9616 |
| 24264 | EGFR-3017 Rz-7 allyl (BAC) | 3017 | 9152 | GAUGUCUACAUGAUC | gₛaₛuₛcₛaugcUAGuGacgccguuaggcgGaaAgacaucB | 9617 |
| 24265 | EGFR-448 Rz-7 allyl (BAC) | 448 | 9157 | GGCUGGUUAUGUCCU | aₛgₛgₛaₛcaucUAGuGacgccguuaggcgGaaAccagccB | 9618 |
| 24266 | EGFR-1455 Rz-7 allyl (BAC) | 1455 | 9167 | AGAACCUAGAAAUCA | uₛgₛaₛuₛuuccUAGuGacgccguuaggcgGaaAgguucuB | 9619 |
| 24267 | EGFR-1506 Rz-6 allyl (BAC) | 1506 | 9169 | UGCAGUCGUCAGC | gₛcₛuₛgₛaccUAGuGacgccguuaggcgGaaAcugcaB | 9620 |
| 24268 | EGFR-3473 Rz-6 allyl (BAC) | 3473 | 9188 | ACCAGUCCGUUCC | gₛgₛaₛaₛcgcUAGuGacgccguuaggcgGaaAcugguB | 9621 |
| 24269 | EGFR-900 CHz-6 allyl (BAC) | 900 | 9203 | UGCUGCAGGCUGC | gₛcₛaₛgₛcccUAGuGacgccguuaggcgGaaIcagcaB | 9622 |
| 24270 | EGFR-1514 CHz-7 allyl (BAC) | 1614 | 9227 | GUCAGCCUGAACAUA | uₛaₛuₛgₛuuccUAGuGacgccguuaggcgGaaIgcugacB | 9623 |
| 24271 | EGFR-2535 CHz-7 allyl (BAC) | 2535 | 9233 | GCCUCACCUCCACCG | cₛgₛgₛuₛggacUAGuGacgccguuaggcgGaaIugaggcB | 9624 |
| 24272 | EGFR-3122 CHz-7 allyl (BAC) | 3122 | 9241 | CGCUACCUUGUCAUU | aₛaₛuₛgₛacacUAGuGacgccguuaggcgGaaIguagcgB | 9625 |
| 24273 | EGFR-3188 CHz-7 allyl (BAC) | 3188 | 9242 | CGUGCCCUGAUGGAU | aₛuₛcₛcₛauccUAGuGacgccguuaggcgGaaIggcacgB | 9626 |

A, G, C, U = Ribo
A, G, C, T (italic)= deoxy
lower case = 2′-O-methyl
s = phosphorothioate 3′-internucleotide linkage
L = hexaethylene glycol spacer
U = 2′-deoxy-2′-C-allyl uridine
U = 2′-deoxy-2′-Amino uridine
C = 2′-deoxy-2′-Amino cytidine
I = Inosine
N = any A, G, C, T or U
B = inverted deoxyabasic derivative

TABLE IX
Comparison of Cell Growth Inhibition by anti-EGFR
nucleic acid molecules

Percent of Untreated

| Cell Type | A549 | SKOv3 | SKOv3 |
|---|---|---|---|
| Treatment | 100 nM | 100 nM | 400 nM |
| Untreated | 100 | 100 | 100 |
| Control GBs | | | |
| 11698 | 18 | 19 | 37 |
| 15340 | 5 | 11 | 14 |
| Control Rzs | | | |
| 21083 | 53 | 23 | 90 |
| 19293 | 17 | 26 | 17 |
| GeneBloc | | | |
| 21542 | 12 | 28 | 27 |
| 21544 | 34 | 33 | 19 |
| Ribozyme | | | |
| 21256 | 13 | 38 | 19 |
| 21261 | 18 | 21 | 41 |
| 21271 | 17 | 18 | 19 |
| 21273 | 20 | 33 | 25 |
| 21293 | 47 | 27 | 25 |
| 21308 | 45 | 22 | 21 |
| 21332 | 47 | 21 | 31 |
| 21338 | 44 | 26 | 53 |
| 21346 | 22 | 30 | 35 |
| 21347 | 22 | 44 | 50 |
| 21439 | 25 | 73 | 23 |
| Tf Control | 45 | 98 | 86 |

TABLE X
Additional Target Sequences shared by EGFR and HER2

| Pos | SeqID | Seq |
|---|---|---|
| 2860 | 325 | ACCCACCAGAGUGAU |
| 3010 | 3518 | GAUGUCUACAUGAUC |
| 3011 | 2916 | AUGUCUACAUGAUCA |
| 3013 | 2494 | GUCUACAUGAUCAUG |
| 3021 | 9128 | UCUACAUGAUCAUGG |
| 3016 | 1516 | UACAUGAUCAUGGUC |
| 3017 | 346 | ACAUGAUCAUGGUCA |
| 3251 | 9254 | CAGCAGGGCUUCUUC |
| 2861 | 9627 | CCCACCAGAGUGAUG |
| 2862 | 9628 | CCACCAGAGUGAUGU |
| 3012 | 9629 | UGUCUACAUGAUCAU |
| 3015 | 9630 | CUACAUGAUCAUGGU |
| 3018 | 9631 | CAUGAUCAUGGUCAA |

What we claim is:

1. An enzymatic nucleic acid molecule which down regulates expression of an epidermal growth factor receptor (EGFR) gene, wherein said enzymatic nucleic acid molecule is in an Inozyme, Zinzyme, G-cleaver, or Amberzyme configuration.

2. An enzymatic nucleic acid molecule comprising a sequence selected from the group consisting of SEQ ID NOs. 3778-9101, 9353-9373, 9376-9597, and 9607-9589.

3. An enzymatic nucleic acid molecule comprising at least one binding arm wherein one or more of said binding arms comprises a sequence complementary to a sequence selected from the group consisting of SEQ ID NOs. 824-3777 and 9102-9352.

4. An antisense nucleic acid molecule comprising a sequence complementary to a sequence selected from the group consisting of SEQ ID NOs. 9335-9342.

5. An antisense nucleic acid molecule comprising a sequence selected from the group consisting of SEQ ID NOs. 9598-9696.

6. The enzymatic nucleic acid of any of claims 1-3, wherein said enzymatic nucleic acid molecule is adapted to treat cancer.

7. The antisense nucleic acid or claim 4 or claim 5, wherein said antisense nucleic acid molecule is adapted to treat cancer.

8. The enzymatic nucleic acid molecule of any of claims 1-3, wherein said enzymatic nucleic acid molecule has an endcnuclease activity to cleave RNA encoded by an EGFR gene.

9. The enzymatic nucleic acid molecule of claim 1, wherein said enzymatic nucleic acid molecule is in an Inozyme configuration.

10. The enzymatic nucleic acid molecule of claim 1, wherein said enzymatic nucleic acid molecule is in a Zinzyme configuration.

11. The enzymatic nucleic acid molecule of claim 1, wherein said enzymatic nucleic acid molecule is in a G-cleaver configuration.

12. The enzymatic nucleic acid molecule of claim 1, wherein said enzymatic nucleic acid molecule is in an Amberzyme configuration.

13. The enzymatic nucleic acid molecule of claim 9, wherein said Inozyme configuration comprises a sequence complementary to a sequence selected from the group consisting of SEQ ID NOs. 824-2117 and 9202-9253.

14. The enzymatic nucleic acid molecule of claim 9, wherein said Inozyme configuration comprises a sequence selected from the group consisting of SEQ ID NOs. 4601-5894, and 9458-9509.

15. The enzymatic nucleic acid molecule of claim 10, wherein said Zinzyme configuration comprises a sequence complementary to a sequence selected from the group consisting of SEQ ID NOs. 2118-2741, 9123, 9126, 9130, 9140, 9147, and 9254-9301.

16. The enzymatic nucleic acid molecule of claim 10, wherein said Zinzyme configuration comprises a sequence selected from the group consisting of SEQ ID NOs 5895-6518, and 9510-9562.

17. The enzymatic nucleic acid molecule of claim 12, wherein said Amberzyme configuration comprises a sequence complementary to a sequence selected from the group consisting of SEQ ID NOs. 2118-2741 and 3068-3777.

18. The enzymatic nucleic acid molecule of claim 12, wherein said Amberzyme configuration comprises a sequence selected from the group consisting of SEQ ID NOs 7768-9101, and 9378-9403.

19. The enzymatic nucleic acid molecule of any of claims 1-3, wherein said enzymatic nucleic acid molecule comprises between 12 and 100 bases complementary to the RNA of EGFR gene.

20. The enzymatic nucleic acid molecule of any of claims 1-3, wherein said enzymatic nucleic acid molecule comprises between 14 and 24 bases complementary to the RNA of EGFR gene.

21. The enzymatic nucleic acid molecule of any of claims 1-3, wherein said enzymatic nucleic acid molecule is chemically synthesized.

22. The antisense nucleic acid molecule of claim 4 or claim 5, wherein said antisense nucleic acid molecule is chemically synthesized.

23. The enzymatic nucleic acid molecule of any of claims 1-3, wherein said enzymatic nucleic acid molecule comprises at least one 2′-sugar modification.

24. The antisense nucleic acid molecule of claim 4, wherein said antisense nucleic acid molecule comprises at least one 2′-sugar modification.

25. The enzymatic nucleic acid molecule of any of claims 1-3, wherein said enzymatic nucleic acid molecule comprises at least one nucleic acid base modification.

26. The antisense nucleic acid molecule of claim 4, wherein said antisense nucleic acid molecule comprises at least one nucleic acid base modification.

27. The enzymatic nucleic acid molecule of any of claims 1-3, wherein said enzymatic nucleic acid molecule comprises at least one phosphate backbone modification.

28. The antisense nucleic acid molecule of claim 4, wherein said antisense nucleic acid molecule comprises at least one phosphate backbone modification.

29. A mammalian cell including the enzymatic nucleic acid molecule of any of claims 1-3.

30. The mammalian cell of claim 29, wherein said mammalian cell is a human cell.

31. A method of reducing EGFR activity in a cell, comprising the step of contacting said cell with the enzymatic nucleic acid molecule of any of claims 1-3, under conditions suitable for said reduction.

32. A method of reducing EGFR activity in a cell, comprising the step of contacting said cell with the antisense nucleic acid molecule of claim 4 or claim 5 under conditions suitable for said reduction.

33. A method of treatment of a patient having a condition associated with the level of EGFR, comprising contacting cells of said patient with the enzymatic nucleic acid molecule of any of claims 1-3, under conditions suitable for said treatment.

34. A method of treatment of a patient having a condition associated with the level of EGFR, comprising contacting cells of said patient with the antisense nucleic acid molecule of claim 4 or claim 5, under conditions suitable for said treatment.

35. The method of claim 31 further comprising the use of one or more drug therapies under conditions suitable for said treatment.

36. The method of claim 32 further comprising the use of one or more drug therapies under conditions suitable for said treatment.

37. The method of claim 33 further comprising the use of one or more drug therapies under conditions suitable for said treatment.

38. The method of claim 34 further comprising the use of one or more drug therapies under conditions suitable for said treatment.

39. A method of cleaving RNA of EGFR gene comprising the step of contacting an enzymatic nucleic acid molecule of any of claims 1-3 with said RNA of EGFR gene under conditions suitable for the cleavage.

40. The method of claim 39, wherein said cleavage is carried out in the presence of a divalent cation.

41. The method of claim 40, wherein said divalent cation is Mg²⁺.

42. The enzymatic nucleic acid molecule of any of claims 1-3, wherein said enzymatic nucleic acid comprises a cap structure, wherein the cap structure is at the 5′-end, or 3′-end, or both the 5′-end and the 3′-end.

43. The antisense nucleic acid molecule of claim 4 or claim 5, wherein said antisense nucleic acid comprises a cap structure, wherein the cap structure is at the 5′-end, or 3′-end, or both the 5′-end and the 3′-end.

44. The enzymatic nucleic acid molecule of claim 42, wherein the cap structure at the 5′-end, 3′-end, or both the 5′-end and the 3′-end comprises a 3′,3′-linked or 5′,5′-linked deoxyabasic ribose derivative.

45. The antisense nucleic acid molecule of claim 43, wherein the cap structure at the 5′-end, 3′-end, or both the 5′-end and the 3′-end comprises a 3′,3′-linked or 5′,5′-linked deoxyabasic ribose derivative.

46. The method of claim 31, wherein said enzymatic nucleic acid molecule is in a Zinzyme configuration.

47. An expression vector comprising a nucleic acid sequence encoding at least one enzymatic nucleic acid molecule of claim 1 or claim 3 in a manner which allows expression of the nucleic acid molecule.

48. A mammalian cell including an expression vector of claim 47.

49. The mammalian cell of claim 48, wherein said mammalian cell is a human cell.

50. The expression vector of claim 47, wherein said enzymatic nucleic acid molecule is in a hammerhead configuration.

51. The expression vector of claim 47, wherein said expression vector further comprises a sequence for an antisense nucleic acid molecule complementary to the RNA of EGFR gene.

52. The expression vector of claim 47, wherein said expression vector comprises a nucleic acid sequence encoding two or more of said enzymatic nucleic acid molecules, which may be the same or different.

53. The expression vector of claim 52, wherein said expression vector further comprises a sequence encoding an antisense nucleic acid molecule complementary to the RNA of EGFR gene.

54. A method for treatment of cancer comprising the step of administering to a patient the enzymatic nucleic acid molecule of any of claims 1-3 under conditions suitable for said treatment.

55. The method of claim 54, wherein said cancer is breast cancer, lung cancer, prostate cancer, colorectal cancer, brain cancer, esophageal cancer, stomach cancer, bladder cancer, pancreatic cancer, cervical cancer, head and neck cancer, ovarian cancer, melanoma, lymphoma, glioma, or multidrug resistant cancer.

56. A method for treatment of cancer comprising the step of administering to a patient the antisense nucleic acid molecule of claim 4 or claim 5 under conditions suitable for said treatment.

57. The method of claim 56, wherein said cancer is breast cancer, lung cancer, prostate cancer, colorectal cancer, brain cancer, esophageal cancer, stomach cancer, bladder cancer, pancreatic cancer, cervical cancer, head and neck cancer, ovarian cancer, melanoma, lymphoma, glioma, or multidrug resistant cancer.

58. The method of claim 54, wherein said enzymatic nucleic acid molecule is in a Zinzyme configuration.

59. The method of claim 54, wherein said method further comprises administering to said patient one or more other therapies.

60. The method of claim 56, wherein said method further comprises administering to said patient one or more other therapies.

61. The nucleic acid molecule of claim 1 or claim 3, wherein said nucleic acid molecule comprises at least five ribose residues, at least ten 2′-O-methyl modifications, and a 3′-end modification.

62. The nucleic acid molecule of claim 61, wherein said nucleic acid molecule further comprises phosphorothioate linkages on at least three of the 5′ terminal nucleotides.

63. The nucleic acid molecule of claim 61, wherein said 3′-end modification is a 3′-3′ inverted abasic moiety.

64. The method of claim 35 wherein said other drug therapies are monoclonal antibodies, EGFR-specific tyrosine kinase inhibitors, or chemotherapy.

65. The method of claim 64, wherein said monoclonal antibodies comprise mAB IMC C225 and mAB ABX-EGF.

66. The method of claim 64, wherein said EGFR-specific tyrosine kinase inhibitors comprise OSI-774 and ZD1839.

67. The method of claim 64, wherein said chemotherapy is paclitaxel, docetaxel, cisplatin, methotrexate, cyclophosphamide, doxorubin, fluorouracil carboplatin, edatrexate, gemcitabine, or vinorelbine.

68. The method of claim 36 wherein said other drug therapies are monoclonal antibodies, EGFR-specific tyrosine kinase inhibitors, or chemotherapy.

69. The method of claim 68, wherein said monoclonal antibodies comprise mAB IMC C225 and mAB ABX-EGF.

70. The method of claim 68, wherein said EGFR-specific tyrosine kinase inhibitors comprise OSI-774 and ZD1839.

71. The method of claim 68, wherein said chemotherapy is paclitaxel, docetaxel, cisplatin, methotrexate, cyclophosphamide, doxorubin, fluorouracil carboplatin, edatrexate, gemcitabine, or vinorelbine.

72. The method of claim 37 wherein said other drug therapies are monoclonal antibodies, EGFR-specific tyrosine kinase inhibitors, or chemotherapy.

73. The method of claim 72, wherein said monoclonal antibodies comprise mAB IMC C225 and mAB ABX-EGF.

74. The method of claim 72, wherein said EGFR-specific tyrosine kinase inhibitors comprise OSI-774 and ZD1839.

75. The method of claim 72, wherein said chemotherapy is paclitaxel, docetaxel, cisplatin, methotrexate, cyclophosphamide, doxorubin, fluorouracil carboplatin, edatrexate, gemcitabine, or vinorelbine.

76. The method of claim 38 wherein said other drug therapies are monoclonal antibodies, EGFR-specific tyrosine kinase inhibitors, or chemotherapy.

77. The method of claim 76, wherein said monoclonal antibodies comprise mAB IMC C225 and mAB ABX-EGF.

78. The method of claim 76, wherein said EGFR-specific tyrosine kinase inhibitors comprise OSI-774 and ZD1839.

79. The method of claim 76, wherein said chemotherapy is paclitaxel, docetaxel, cisplatin, methotrexate, cyclophosphamide, doxorubin, fluorouracil carboplatin, edatrexate, gemcitabine, or vinorelbine.

80. The method of claim 59, wherein said other therapies are monoclonal antibodies, EGFR-specific tyrosine kinase inhibitors (TKIs), chemotherapy, or radiation therapy.

81. The method of claim 80, wherein said monoclonal antibodies comprise mAB IMC C225 and mAB ABX-EGF.

82. The method of claim 80, wherein said EGFR-specific tyrosine kinase inhibitors comprise OSI-774 and ZD1839.

83. The method of claim 80, wherein said chemotherapy is paclitaxel, docetaxel, cisplatin, methotrexate, cyclophosphamide, doxorubin, fluorouracil carboplatin, edatrexate, gemcitabine, or vinorelbine.

84. The method of claim 60, wherein said other therapies are monoclonal antibodies, EGFR-specific tyrosine kinase inhibitors (TKIs), chemotherapy, or radiation therapy.

85. The method of claim 84, wherein said monoclonal antibodies comprise mAB IMC C225 and mAB ABX-EGF.

86. The method of claim 84, wherein said EGFR-specific tyrosine kinase inhibitors comprise OSI-774 and ZD1839.

87. The method of claim 84, wherein said chemotherapy is paclitaxel, docetaxel, cisplatin, methotrexate, cyclophosphamide, doxorubin, fluorouracil carboplatin, edatrexate, gemcitabine, or vinorelbine.

88. A nucleic acid molecule which down regulates expression of both an epidermal growth factor receptor (EGFR) gene and a HER2 gene.

89. The nucleic acid of claim 88, wherein said nucleic acid molecule is adapted to treat cancer.

90. The nucleic acid molecule of claim 89, wherein the cancer is breast cancer, lung cancer, prostate cancer, colorectal cancer, brain cancer, esophageal cancer, stomach cancer, bladder cancer, pancreatic cancer, cervical cancer, head and neck cancer, ovarian cancer, melanoma, lymphoma, glioma, or multidrug resistant cancer.

91. The nucleic acid molecule of claim 88, wherein said nucleic acid molecule is an enzymatic nucleic acid molecule having at least one binding arm.

92. The nucleic acid molecule of claim 91, wherein said enzymatic nucleic acid molecule has an endonuclease activity to cleave RNA encoded by both an epidermal growth factor receptor (EGFR) and a HER2 gene.

93. The nucleic acid of claim 91, wherein one or more binding arms of the enzymatic nucleic acid molecule comprises a sequence complementary to a sequence having SEQ ID NOs. 325, 346, 1516, 2494, 2916, 3518, 9128, 9254, 9627, 9628, 9629, 9630, or 9631.

94. An enzymatic nucleic acid molecule comprising a sequence having SEQ ID NOs. 4102, 4123, 5292, 6271, 7183, 7215, 7216, 7217, 8594, 8595, 9383, 9406, 9407, 9463, or 9513.

95. The nucleic acid molecule of claim 88, wherein said nucleic acid molecule is an antisense nucleic acid molecule.

96. An antisense nucleic acid molecule comprising a sequence complementary to a sequence having SEQ ID NOs. 325, 346, 1516, 2494, 2916, 3518, 9128, 9254, 9627, 9628, 9629, 9630, or 9631.

97. The enzymatic nucleic acid molecule of claim 92, wherein said enzymatic nucleic acid molecule is in a hammerhead (HH) configuration.

98. The enzymatic nucleic acid molecule of claim 92, wherein said enzymatic nucleic acid molecule is in a hairpin, hepatitis Delta virus, group I intron, VS nucleic acid, amberzyme, zinzyme or RNAse P nucleic acid configuration.

99. The enzymatic nucleic acid molecule of claim 4, wherein said enzymatic nucleic acid molecule is in a NCH configuration.

100. The enzymatic nucleic acid molecule of claim 4, wherein said enzymatic nucleic acid molecule is in a G-cleaver configuration.

101. The enzymatic nucleic acid molecule of claim 4, wherein said enzymatic nucleic acid molecule is a DNAzyme.

102. The nucleic acid molecule of claim 88, wherein said nucleic acid molecule comprises between 12 and 100 bases complementary to the RNA of both EGFR and HER2 genes.

103. The nucleic acid molecule of claim 88, wherein said nucleic acid molecule comprises between 14 and 24 bases complementary to the RNA of EGFR and HER2 genes.

104. The nucleic acid molecule of claim 88, wherein said nucleic acid molecule is chemically synthesized.

105. The nucleic acid molecule of claim 88, wherein said nucleic acid molecule comprises at least one 2′-sugar modification.

106. The nucleic acid molecule of claim 88, wherein said nucleic acid molecule comprises at least one nucleic acid base modification.

107. The nucleic acid molecule of claim 88, wherein said nucleic acid molecule comprises at least one phosphate backbone modification.

108. A mammalian cell including the nucleic acid molecule of claim 88.

109. The mammalian cell of claim 108, wherein said mammalian cell is a human cell.

110. A method of reducing EGFR and HER2 activity in a cell, comprising the step of contacting said cell with a nucleic acid molecule of claim 1, under conditions suitable for said reduction.

111. A method of treatment of a patient having a condition associated with the level of EGFR and HER2, comprising contacting cells of said patient with a nucleic acid molecule of claim 1, under conditions suitable for said treatment.

112. The method of claim 111 further comprising the use of one or more drug therapies under conditions suitable for the treatment.

113. A method of cleaving RNA of both EGFR and HER2 genes comprising the step of contacting a nucleic acid molecule of claim 91 with said RNA under conditions suitable for the cleavage of said RNA.

114. The method of claim 113, wherein said cleavage is carried out in the presence of a divalent cation.

115. The method of claim 114, wherein said divalent cation is Mg²⁺.

116. The nucleic acid molecule of claim 88, wherein said nucleic acid comprises a cap structure, wherein the cap structure is at the 5′-end, 3′-end, or both the 5′-end and the 3′-end.

117. The method of claim 110, wherein said nucleic acid molecule is an enzymatic nucleic acid molecule.

118. The method of claim 117, wherein said enzymatic nucleic acid molecule is in a hammerhead configuration.

119. The method of claim 117, wherein said enzymatic nucleic acid molecule is a DNAzyme.

120. The method of claim 110, wherein said nucleic acid molecule is an antisense nucleic acid molecule.

121. An expression vector comprising a nucleic acid sequence encoding at least one nucleic acid molecule of claim 88 in a manner which allows expression of the nucleic acid molecule.

122. A mammalian cell including an expression vector of claim 121.

123. The mammalian cell of claim 122, wherein said mammalian cell is a human cell.

124. The expression vector of claim 121, wherein said nucleic acid molecule is an enzymatic nucleic acid molecule.

125. The expression vector of claim 121, wherein said nucleic acid molecule is an antisense nucleic acid molecule.

126. The expression vector of claim 121, wherein said expression vector further comprises a sequence for an antisense nucleic acid molecule complementary to the RNA of both EGFR and HER2 genes.

127. The expression vector of claim 121, wherein said expression vector further comprises a nucleic acid sequence encoding two or more of said nucleic acid molecules, which may be the same or different.

128. The expression vector of claim 127, wherein said expression vector comprises a sequence encoding antisense nucleic acid molecule complementary to the RNA of both EGFR and HER2 genes.

129. A method for treatment cancer comprising the step of administering to a patient the nucleic acid molecule of claim 88 under conditions suitable for said treatment.

130. The method of claim 129, wherein said cancer is breast cancer, lung cancer, prostate cancer, colorectal cancer, brain cancer, esophageal cancer, stomach cancer, bladder cancer, pancreatic cancer, cervical cancer, head and neck cancer, ovarian cancer, melanoma, lymphoma, glioma, or multidrug resistant cancer.

131. The method of claim 129, wherein said nucleic acid molecule is in a hammerhead configuration.

132. The method of claim 129. wherein said method further comprises administering to said patient one or more other therapies.

133. The nucleic acid molecule of claim 88, wherein said nucleic acid molecule comprises at least five ribose residues, at least ten 2′-O-methyl modifications, and a 3′-end modification.

134. The nucleic acid molecule of claim 131, wherein said nucleic acid molecule further comprises phosphorothioate linkages on at least three of the 5′ terminal nucleotides.

135. The nucleic acid molecule of claim 131, wherein said 3′-end modification is 3′-3′ inverted abasic moiety.

136. The method of claim 119, wherein said DNAzyme comprises at least ten 2′-O-methyl modifications and a 3′-end modification.

137. The method of claim 136, wherein said DNAzyme further comprises phosphorothioate linkages on at least three of the 5′ terminal nucleotides.

138. The method of claim 136, wherein said 3′-end modification is 3′-3′ inverted abasic moiety.

139. The method of claim 132, wherein said other therapies are monoclonal antibodies, EGFR-specific tyrosine kinase inhibitors (TKIs), chemotherapy, or radiation therapy.

140. The method of claim 139, wherein said monoclonal antibodies comprise mAB IMC C225 and mAB ABX-EGF.

141. The method of claim 139, wherein said EGFR-specific tyrosine kinase inhibitors comprise OSI-774 and ZD1839.

142. The method of claim 139, wherein said chemotherapy is paclitaxel, docetaxel, cisplatin, methotrexate, cyclophosphamide, doxorubin, fluorouracil carboplatin, edatrexate, gemcitabine, or vinorelbine.

143. A pharmaceutical composition comprising an enzymatic nucleic acid molecule of any of claims 1-3.

144. A pharmaceutical composition comprising an antisense nucleic acid molecule of claim 4 or claim 5.

145. A pharmaceutical composition comprising an enzymatic nucleic acid molecule of claim 88.
